(19) United States
(12) Patent Application Publication
(10) Pub. No.: US 2020/0299774 A1
(43) Pub. Date: Sep. 24, 2020

(54) MOLECULAR PROFILING FOR CANCER

(71) Applicant: Caris MPI, Inc., Irving, TX (US)

(72) Inventors: Gargi BASU, Scottsdale, AZ (US); David ARGUELLO, Phoenix, AZ (US); Rebecca FELDMAN, Chandler, AZ (US); Xinan XIU, Chandler, AZ (US); Zoran GATALICA, Paradise Valley, AZ (US)

(73) Assignee: Caris MPI, Inc., Irving, TX (US)

(21) Appl. No.: 16/597,061

(22) Filed: Oct. 9, 2019

Related U.S. Application Data
(63) Continuation of application No. 14/648,988, filed on Jun. 2, 2015, now abandoned.
(60) Provisional application No. 61/904,398, filed on Nov. 14, 2013.
(60) Provisional application No. 61/887,971, filed on Oct. 7, 2013.
(60) Provisional application No. 61/885,456, filed on Oct. 1, 2013.
(60) Provisional application No. 61/879,498, filed on Sep. 18, 2013.
(60) Provisional application No. 61/878,536, filed on Sep. 16, 2013.
(60) Provisional application No. 61/865,957, filed on Aug. 14, 2013.
(60) Provisional application No. 61/847,057, filed on Jul. 16, 2013.
(60) Provisional application No. 61/830,018, filed on May 31, 2013.
(60) Provisional application No. 61/759,986, filed on Feb. 1, 2013.
(60) Provisional application No. 61/757,701, filed on Jan. 28, 2013.
(60) Provisional application No. 61/733,396, filed on Dec. 4, 2012.

(51) Int. Cl. C12Q 1/6886; G16B 25/00; G16B 40/00; G01N 33/574; C12Q 1/6869; G16H 50/20

(52) U.S. Cl. CPC C12Q 1/6886; G16B 25/00; G16B 40/00; G01N 33/57484; C12Q 1/6869; C12Q 2600/16; C12Q 2600/106; C12Q 2600/156; Y02A 90/26; C12Q 2600/112; C12Q 2600/154; G16H 50/20

(57) ABSTRACT

Provided herein are methods and systems of molecular profiling of diseases, such as cancer. In some embodiments, the molecular profiling can be used to identify treatments that have likely benefit for a cancer, such as treatments that were not initially identified as a treatment for the disease or not expected to be a treatment for a particular disease. The molecular profiling can be used to identify likely have lack of benefit for treating the cancer.

### CROSS REFERENCE

This application claims the benefit of U.S. Provisional Patent Application No. 61/733,396, filed Dec. 4, 2012; 61/757,701, filed Jan. 28, 2013; 61/759,986, filed Feb. 1, 2013; 61/830,018, filed May 31, 2013; 61/847,057, filed Jul. 16, 2013; 61/865,957, filed Aug. 14, 2013; 61/878,536, filed Sep. 16, 2013; 61/879,498, filed Sep. 18, 2013; 61/885,456, filed Oct. 1, 2013; 61/887,971, filed Oct. 7, 2013; 61/904,398, filed Nov. 14, 2013; all of which applications are incorporated herein by reference in their entirety.

### BACKGROUND

Disease states in patients are typically treated with treatment regimens or therapies that are selected based on clinical based criteria; that is, a treatment therapy or regimen is selected for a patient based on the determination that the patient has been diagnosed with a particular disease (which diagnosis has been made from classical diagnostic assays). Although the molecular mechanisms behind various disease states have been the subject of studies for years, the specific application of a diseased individual's molecular profile in determining treatment regimens and therapies for that individual has been disease specific and not widely pursued.

Some treatment regimens have been determined using molecular profiling in combination with clinical characterization of a patient such as observations made by a physician (such as a code from the International Classification of Diseases, for example, and the dates such codes were determined), laboratory test results, x-rays, biopsy results, statements made by the patient, and any other medical information typically relied upon by a physician to make a diagnosis in a specific disease. However, using a combination of selection material based on molecular profiling and clinical characterizations (such as the diagnosis of a particular type of cancer) to determine a treatment regimen or therapy presents a risk that an effective treatment regimen may be overlooked for a particular individual since some treatment regimens may work well for different disease states even though they are associated with treating a particular type of disease state.

Patients with refractory or metastatic cancer are of particular concern for treating physicians. The majority of patients with metastatic or refractory cancer eventually run out of treatment options or may suffer a cancer type with no real treatment options. For example, some patients have very limited options after their tumor has progressed in spite of front line, second line and sometimes third line and beyond) therapies. For these patients, molecular profiling of their cancer may provide the only viable option for prolonging life.

More particularly, additional targets or specific therapeutic agents can be identified assessment of a comprehensive number of targets or molecular findings examining molecular mechanisms, genes, gene expressed proteins, and/or combinations of such in a patient's tumor. Identifying multiple agents that can treat multiple targets or underlying mechanisms would provide cancer patients with a viable therapeutic alternative on a personalized basis so as to avoid standar therapies, which may simply not work or identify therapies that would not otherwise be considered by the treating physician.

There remains a need for better theranostic assessment of cancer vicitims, including molecular profiling analysis that identifies one or more individual profiles to provide more informed and effective personalized treatment options, resulting in improved patient care and enhanced treatment outcomes. The present invention provides methods and systems for identifying treatments for these individuals by molecular profiling a sample from the individual.

### SUMMARY OF THE INVENTION

The present invention provides methods and system for molecular profiling, using the results from molecular profiling to identify treatments for individuals. In some embodiments, the treatments were not identified initially as a treatment for the disease or disease lineage.

In an aspect, the invention provides a method of identifying one or more candidate treatment for a cancer in a subject in need thereof, comprising: (a) determining a molecular profile for a sample from the subject by assessing a panel of gene or gene products, wherein the panel of gene or gene products are assessed as indicated in Table 21, FIG. 33A or FIG. 33B; and (b) identifying one or more treatment that is beneficially associated with the molecular profile of the subject, and optionally one or more treatment associated with lack of benefit, according to the determining in (a) and one or more rules in Table 22, thereby identifying the one or more candidate treatment. The panel of gene or gene products may comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57 or 58, of: ABL1, AKT1, ALK, APC, AR, ATM, BRAF, CDH1, cKIT, cMET, CSF1R, CTNNB1, EGFR, ER, ERBB2, ERBB4, FBXW7, FGFR1, FGFR2, FLT3, GNA11, GNAQ, GNAS, HER2, HNF1A, HRAS, IDH1, JAK2, JAK3, KDR (VEGFR2), KRAS, MGMT, MLH1, MPL, NOTCH1, NPM1, NRAS, PDGFRA, PGP, PIK3CA, PR, PTEN, PTPN11, RB1, RET, RRM1, SMAD4, SMARCB1, SMO, SPARC, STK11, TLE3, TOP2A, TOPO1, TP53, TS, TUBB3 and VHL. Assessing the panel of gene or gene products may comprise using ISH to assess 1 or 2 of cMET and HER2. Assessing the panel of gene or gene products may comprise using IHC to assess 1, 2, 3, 4, 5, 6, 7, 8, 9, 10, 11, 12, 13, 14, 15 or 16 of AR, cMET, ER, HER2, MGMT, PGP, PR, PTEN, RRM1, SPARCm, SPARCp, TLE3, TOP2A, TOPO1, TS, TUBB3. Assessing the panel of gene or gene products may comprise using sequence analysis to assess 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33 or 34 of: ABL1, AKT1, ALK, APC, ATM, BRAF, cKIT, cMET, CSF1R, CTNNB1, EGFR, ERBB2, FGFR1, FGFR2, FLT3, GNA11, GNAQ, GNAS, HRAS, IDH1, JAK2, KDR (VEGFR2), KRAS, MLH1, MPL, NOTCH1, NRAS, PDGFRA, PIK3CA, PTEN, RET, SMO, TP53, VHL. Assessing the panel of gene or gene products may comprise using ISH to assess 1 or 2 of cMET and HER2; using IHC to assess 1, 2, 3, 4, 5, 6, 7, 8, 9, 10, 11, 12, 13, 14, 15 or 16 of AR, cMET, ER, HER2, MGMT, PGP, PR, PTEN, RRM1, SPARCm, SPARCp, TLE3, TOP2A, TOPO1, TS, TUBB3; and/or comprises using sequence analysis to assess 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33 or 34 of: ABL1, AKT1, ALK, APC, ATM, BRAF, cKIT, cMET, CSF1R, CTNNB1, EGFR, ERBB2, FGFR1, FGFR2, FLT3, GNA11, GNAQ, GNAS, HRAS, IDH1, JAK2, KDR (VEGFR2), KRAS, MLH1, MPL, NOTCH1, NRAS, PDGFRA, PIK3CA, PTEN, RET, SMO, TP53, VHL. Assessing the panel of gene or gene products may comprise using sequence analysis to assess 1, 2, 3, 4, 5, 6, 7, 8, 9, 10 or 11 of CDH1, ERBB4, FBXW7, HNF1A, JAK3, NPM1, PTPN11, RB1, SMAD4, SMARCB1 and STK11. The sequence analysis can be performed using Next Generation Sequencing.

In some embodiments, the panel of gene or gene products comprises the androgen receptor (AR). In such cases, the one or more candidate treatment can be an antiandrogen. The antiandrogen may suppress androgen production and/or inhibits androgens from binding to AR. The antiandrogen can be one or more of abarelix, bicalutamide, flutamide, gonadorelin, goserelin, leuprolide, nilutamide, a 5-alpha-reductase inhibitor, finasteride, dutasteride, bexlosteride, izonsteride, turosteride, and epristeride. The cancer can be androgen independent. In embodiments, the one or more candidate treatment comprises one or more of a CYP17 inhibitor, CYP17A1 inhibitor, chemotherapeutic agent, antiandrogen, an endocrine disruptor, immunotherapy, and bone-targeting radiopharmaceutical.

The methods of the invention can be used to profile any cancer. For example, the cancer may comprise an acute lymphoblastic leukemia; acute myeloid leukemia; adrenocortical carcinoma; AIDS-related cancer; AIDS-related lymphoma; anal cancer; appendix cancer; astrocytomas; atypical teratoid/rhabdoid tumor; basal cell carcinoma; bladder cancer; brain stem glioma; brain tumor, brain stem glioma, central nervous system atypical teratoid/rhabdoid tumor, central nervous system embryonal tumors, astrocytomas, craniopharyngioma, ependymoblastoma, ependymoma, medulloblastoma, medulloepithelioma, pineal parenchymal tumors of intermediate differentiation, supratentorial primitive neuroectodermal tumors and pineoblastoma; breast cancer; bronchial tumors; Burkitt lymphoma; cancer of unknown primary site (CUP); carcinoid tumor; carcinoma of unknown primary site; central nervous system atypical teratoid/rhabdoid tumor; central nervous system embryonal tumors; cervical cancer; childhood cancers; chordoma; chronic lymphocytic leukemia; chronic myelogenous leukemia; chronic myeloproliferative disorders; colon cancer; colorectal cancer; craniopharyngioma; cutaneous T-cell lymphoma; endocrine pancreas islet cell tumors; endometrial cancer; ependymoblastoma; ependymoma; esophageal cancer; esthesioneuroblastoma; Ewing sarcoma; extracranial germ cell tumor; extragonadal germ cell tumor; extrahepatic bile duct cancer; gallbladder cancer; gastric (stomach) cancer; gastrointestinal carcinoid tumor; gastrointestinal stromal cell tumor; gastrointestinal stromal tumor (GIST); gestational trophoblastic tumor; glioma; hairy cell leukemia; head and neck cancer; heart cancer; Hodgkin lymphoma; hypopharyngeal cancer; intraocular melanoma; islet cell tumors; Kaposi sarcoma; kidney cancer; Langerhans cell histiocytosis; laryngeal cancer; lip cancer; liver cancer; malignant fibrous histiocytoma bone cancer; medulloblastoma; medulloepithelioma; melanoma; Merkel cell carcinoma; Merkel cell skin carcinoma; mesothelioma; metastatic squamous neck cancer with occult primary; mouth cancer; multiple endocrine neoplasia syndromes; multiple myeloma; multiple myeloma/plasma cell neoplasm; mycosis fungoides; myelodysplastic syndromes; myeloproliferative neoplasms; nasal cavity cancer; nasopharyngeal cancer; neuroblastoma; Non-Hodgkin lymphoma; nonmelanoma skin cancer; non-small cell lung cancer; oral cancer; oral cavity cancer; oropharyngeal cancer; osteosarcoma; other brain and spinal cord tumors; ovarian cancer; ovarian epithelial cancer; ovarian germ cell tumor; ovarian low malignant potential tumor; pancreatic cancer; papillomatosis; paranasal sinus cancer; parathyroid cancer; pelvic cancer; penile cancer; pharyngeal cancer; pineal parenchymal tumors of intermediate differentiation; pineoblastoma; pituitary tumor; plasma cell neoplasm/multiple myeloma; pleuropulmonary blastoma; primary central nervous system (CNS) lymphoma; primary hepatocellular liver cancer; prostate cancer; rectal cancer; renal cancer; renal cell (kidney) cancer; renal cell cancer; respiratory tract cancer; retinoblastoma; rhabdomyosarcoma; salivary gland cancer; Sézary syndrome; small cell lung cancer; small intestine cancer; soft tissue sarcoma; squamous cell carcinoma; squamous neck cancer; stomach (gastric) cancer; supratentorial primitive neuroectodermal tumors; T-cell lymphoma; testicular cancer; throat cancer; thymic carcinoma; thymoma; thyroid cancer; transitional cell cancer; transitional cell cancer of the renal pelvis and ureter; trophoblastic tumor; ureter cancer; urethral cancer; uterine cancer; uterine sarcoma; vaginal cancer; vulvar cancer; Waldenström macroglobulinemia; or Wilm's tumor. The cancer can be an acute myeloid leukemia (AML), breast carcinoma, cholangiocarcinoma, colorectal adenocarcinoma, extrahepatic bile duct adenocarcinoma, female genital tract malignancy, gastric adenocarcinoma, gastroesophageal adenocarcinoma, gastrointestinal stromal tumor (GIST), glioblastoma, head and neck squamous carcinoma, leukemia, liver hepatocellular carcinoma, low grade glioma, lung bronchioloalveolar carcinoma (BAC), non-small cell lung cancer (NSCLC), lung small cell cancer (SCLC), lymphoma, male genital tract malignancy, malignant solitary fibrous tumor of the pleura (MSFT), melanoma, multiple myeloma, neuroendocrine tumor, nodal diffuse large B-cell lymphoma, non epithelial ovarian cancer (non-EOC), ovarian surface epithelial carcinoma, pancreatic adenocarcinoma, pituitary carcinomas, oligodendroglioma, prostatic adenocarcinoma, retroperitoneal or peritoneal carcinoma, retroperitoneal or peritoneal sarcoma, small intestinal malignancy, soft tissue tumor, thymic carcinoma, thyroid carcinoma, or uveal melanoma. In some embodiments, the cancer comprises a prostate, bladder, kidney, lung, breast, or liver cancer.

In an aspect, the invention provides a method of identifying one or more candidate treatment for an ovarian cancer in a subject in need thereof, comprising: (a) determining a molecular profile for a sample from the subject by assessing a panel of gene or gene products, wherein the panel of gene or gene products are assessed as indicated in Table 7, FIG. 33C or FIG. 33D; and (b) identifying one or more treatment that is beneficially associated with the molecular profile of the subject, and optionally one or more treatment associated with lack of benefit, according to the determining in (a) and one or more rules in Table 8, thereby identifying the one or more candidate treatment. The panel of gene or gene products can include 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57 or 58, of: ABL1, AKT1, ALK, APC, AR, ATM, BRAF, CDH1, cKIT, cMET, CSF1R, CTNNB1, EGFR, ER, ERBB2, ERBB4, FBXW7, FGFR1, FGFR2, FLT3, GNA11, GNAQ, GNAS, HER2, HNF1A, HRAS, IDH1, JAK2, JAK3, KDR (VEGFR2), KRAS, MGMT, MLH1, MPL, NOTCH1, NPM1, NRAS, PDGFRA, PGP, PIK3CA, PR, PTEN, PTPN11, RB1, RET, RRM1, SMAD4, SMARCB1, SMO, SPARC, STK11, TLE3, TOP2A, TOPO1, TP53, TS, TUBB3, VHL. Assessing the panel of gene or gene products may comprise using ISH to assess 1 or 2 of cMET and HER2. Assessing the panel of gene or gene products may comprise using IHC to assess 1, 2, 3, 4, 5, 6, 7, 8, 9, 10, 11, 12, 13, 14, 15 or 16 of: AR, cMET, ER, HER2, MGMT, PGP, PR, PTEN, RRM1, SPARCm, SPARCp, TLE3, TOP2A, TOPO1, TS, TUBB3. Assessing the panel of gene or gene products may comprise using sequence analysis to assess 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33 or 34 of: ABL1, AKT1, ALK, APC, ATM, BRAF, cKIT, cMET, CSF1R, CTNNB1, EGFR, ERBB2, FGFR1, FGFR2, FLT3, GNA11, GNAQ, GNAS, HRAS, IDH1, JAK2, KDR (VEGFR2), KRAS, MLH1, MPL, NOTCH1, NRAS, PDGFRA, PIK3CA, PTEN, RET, SMO, TP53, VHL. Assessing the panel of gene or gene products may comprise using ISH to assess 1 or 2 of cMET and HER2; using IHC to assess 1, 2, 3, 4, 5, 6, 7, 8, 9, 10, 11, 12, 13, 14, 15 or 16 of: AR, cMET, ER, HER2, MGMT, PGP, PR, PTEN, RRM1, SPARCm, SPARCp, TLE3, TOP2A, TOPO1, TS, TUBB3; and/or using sequence analysis to assess 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33 or 34 of: ABL1, AKT1, ALK, APC, ATM, BRAF, cKIT, cMET, CSF1R, CTNNB1, EGFR, ERBB2, FGFR1, FGFR2, FLT3, GNA11, GNAQ, GNAS, HRAS, IDH1, JAK2, KDR (VEGFR2), KRAS, MLH1, MPL, NOTCH1, NRAS, PDGFRA, PIK3CA, PTEN, RET, SMO, TP53, VHL. Assessing the panel of gene or gene products may comprise using sequence analysis to assess 1, 2, 3, 4, 5, 6, 7, 8, 9, 10 or 11 of CDH1, ERBB4, FBXW7, HNF1A, JAK3, NPM1, PTPN11, RB1, SMAD4, SMARCB1 and STK11. In some embodiments, the sequence analysis comprises Next Generation Sequencing.

In an aspect, the invention provides a method of identifying one or more candidate treatment for a breast cancer in a subject in need thereof, comprising: (a) determining a molecular profile for a sample from the subject by assessing a panel of gene or gene products, wherein the panel of gene or gene products are assessed as indicated in Table 9, FIG. 33K or FIG. 33L; and (b) identifying one or more treatment that is beneficially associated with the molecular profile of the subject, and optionally one or more treatment associated with lack of benefit, according to the determining in (a) and one or more rules in Table 10, thereby identifying the one or more candidate treatment. The panel of gene or gene products can include 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57 or 58, of: ABL1, AKT1, ALK, APC, AR, ATM, BRAF, CDH1, cKIT, cMET, CSF1R, CTNNB1, EGFR, ER, ERBB2, ERBB4, FBXW7, FGFR1, FGFR2, FLT3, GNA11, GNAQ, GNAS, HER2, HNF1A, HRAS, IDH1, JAK2, JAK3, KDR (VEGFR2), KRAS, MGMT, MLH1, MPL, NOTCH1, NPM1, NRAS, PDGFRA, PGP, PIK3CA, PR, PTEN, PTPN11, RB1, RET, RRM1, SMAD4, SMARCB1, SMO, SPARC, STK11, TLE3, TOP2A, TOPO1, TP53, TS, TUBB3, VHL. Assessing the panel of gene or gene products may comprise using ISH to assess 1, 2 or 3, of: cMET, HER2, TOP2A. Assessing the panel of gene or gene products may comprise using IHC to assess 1, 2, 3, 4, 5, 6, 7, 8, 9, 10, 11, 12, 13, 14, 15 or 16 of: AR, cMET, ER, HER2, MGMT, PGP, PR, PTEN, RRM1, SPARCm, SPARCp, TLE3, TOPO1, TS, TUBB3. Assessing the panel of gene or gene products may comprise using sequence analysis to assess 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33 or 34 of: ABL1, AKT1, ALK, APC, ATM, BRAF, cKIT, cMET, CSF1R, CTNNB1, EGFR, ERBB2, FGFR1, FGFR2, FLT3, GNA11, GNAQ, GNAS, HRAS, IDH1, JAK2, KDR (VEGFR2), KRAS, MLH1, MPL, NOTCH1, NRAS, PDGFRA, PIK3CA, PTEN, RET, SMO, TP53, VHL. Assessing the panel of gene or gene products may comprise using ISH to assess 1, 2 or 3, of: cMET, HER2, TOP2A; using IHC to assess 1, 2, 3, 4, 5, 6, 7, 8, 9, 10, 11, 12, 13, 14, 15 or 16 of: AR, cMET, ER, HER2, MGMT, PGP, PR, PTEN, RRM1, SPARCm, SPARCp, TLE3, TOPO1, TS, TUBB3; and/or using sequence analysis to assess 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33 or 34 of: ABL1, AKT1, ALK, APC, ATM, BRAF, cKIT, cMET, CSF1R, CTNNB1, EGFR, ERBB2, FGFR1, FGFR2, FLT3, GNA11, GNAQ, GNAS, HRAS, IDH1, JAK2, KDR (VEGFR2), KRAS, MLH1, MPL, NOTCH1, NRAS, PDGFRA, PIK3CA, PTEN, RET, SMO, TP53, VHL. Assessing the panel of gene or gene products may comprise using sequence analysis to assess 1, 2, 3, 4, 5, 6, 7, 8, 9, 10 or 11 of CDH1, ERBB4, FBXW7, HNF1A, JAK3, NPM1, PTPN11, RB1, SMAD4, SMARCB1 and STK11. In some embodiments, the sequence analysis comprises Next Generation Sequencing.

In an aspect, the invention provides a method of identifying one or more candidate treatment for a skin cancer (melanoma) in a subject in need thereof, comprising: (a) determining a molecular profile for a sample from the subject by assessing a panel of gene or gene products, wherein the panel of gene or gene products are assessed as indicated in Table 11, FIG. 33E or FIG. 33F; and (b) identifying one or more treatment that is beneficially associated with the molecular profile of the subject, and optionally one or more treatment associated with lack of benefit, according to the determining in (a) and one or more rules in Table 12, thereby identifying the one or more candidate treatment. The panel of gene or gene products can include 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57 or 58, of: ABL1, AKT1, ALK, APC, AR, ATM, BRAF, CDH1, cKIT, cMET, CSF1R, CTNNB1, EGFR, ER, ERBB2, ERBB4, FBXW7, FGFR1, FGFR2, FLT3, GNA11, GNAQ, GNAS, HER2, HNF1A, HRAS, IDH1, JAK2, JAK3, KDR (VEGFR2), KRAS, MGMT, MLH1, MPL, NOTCH1, NPM1, NRAS, PDGFRA, PGP, PIK3CA, PR, PTEN, PTPN11, RB1, RET, RRM1, SMAD4, SMARCB1, SMO, SPARC, STK11, TLE3, TOP2A, TOPO1, TP53, TS, TUBB3, VHL. Assessing the panel of gene or gene products may comprise using ISH to assess 1 or 2 of: cMET, HER2. Assessing the panel of gene or gene products may comprise using IHC to assess 1, 2, 3, 4, 5, 6, 7, 8, 9, 10, 11, 12, 13, 14, 15 or 16 of: AR, cMET, ER, HER2, MGMT, PGP, PR, PTEN, RRM1, SPARCm, SPARCp, TLE3, TOP2A, TOPO1, TS, TUBB3. Assessing the panel of gene or gene products may comprise using sequence analysis to assess 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33 or 34 of: ABL1, AKT1, ALK, APC, ATM, BRAF, cKIT, cMET, CSF1R, CTNNB1, EGFR, ERBB2, FGFR1, FGFR2, FLT3, GNA11, GNAQ, GNAS, HRAS, IDH1, JAK2, KDR (VEGFR2), KRAS, MLH1, MPL, NOTCH1, NRAS, PDGFRA, PIK3CA, PTEN, RET, SMO, TP53, VHL. Assessing the panel of gene or gene products may comprise using ISH to assess 1 or 2 of: cMET, HER2; using IHC to assess 1, 2, 3, 4, 5, 6, 7, 8, 9, 10, 11, 12, 13, 14, 15 or 16 of: AR, cMET, ER, HER2, MGMT, PGP, PR, PTEN, RRM1, SPARCm, SPARCp, TLE3, TOP2A, TOPO1, TS, TUBB3; and/or using sequence analysis to assess 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33 or 34 of: ABL1, AKT1, ALK, APC, ATM, BRAF, cKIT, cMET, CSF1R, CTNNB1, EGFR, ERBB2, FGFR1, FGFR2, FLT3, GNA11, GNAQ, GNAS, HRAS, IDH1, JAK2, KDR (VEGFR2), KRAS, MLH1, MPL, NOTCH1, NRAS, PDGFRA, PIK3CA, PTEN, RET, SMO, TP53, VHL. Assessing the panel of gene or gene products may comprise using sequence analysis to assess 1, 2, 3, 4, 5, 6, 7, 8, 9, 10 or 11 of CDH1, ERBB4, FBXW7, HNF1A, JAK3, NPM1, PTPN11, RB1, SMAD4, SMARCB1 and STK11. In some embodiments, the sequence analysis comprises Next Generation Sequencing. In various embodiments, the sequence analysis of BRAF comprises PCR, e.g., the FDA approved cobas PCR assay.

In an aspect, the invention provides a method of identifying one or more candidate treatment for a uveal melanoma cancer in a subject in need thereof, comprising: (a) determining a molecular profile for a sample from the subject by assessing a panel of gene or gene products, wherein the panel of gene or gene products are assessed as indicated in Table 13, FIG. 33G or FIG. 33H; and (b) identifying one or more treatment that is beneficially associated with the molecular profile of the subject, and optionally one or more treatment associated with lack of benefit, according to the determining in (a) and one or more rules in Table 14, thereby identifying the one or more candidate treatment. The panel of gene or gene products can include 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57 or 58, of: ABL1, AKT1, ALK, APC, AR, ATM, BRAF, CDH1, cKIT, cMET, CSF1R, CTNNB1, EGFR, ER, ERBB2, ERBB4, FBXW7, FGFR1, FGFR2, FLT3, GNA11, GNAQ, GNAS, HER2, HNF1A, HRAS, IDH1, JAK2, JAK3, KDR (VEGFR2), KRAS, MGMT, MLH1, MPL, NOTCH1, NPM1, NRAS, PDGFRA, PGP, PIK3CA, PR, PTEN, PTPN11, RB1, RET, RRM1, SMAD4, SMARCB1, SMO, SPARC, STK11, TLE3, TOP2A, TOPO1, TP53, TS, TUBB3, VHL. Assessing the panel of gene or gene products may comprise using ISH to assess 1 or 2, of: cMET, HER2. Assessing the panel of gene or gene products may comprise using IHC to assess 1, 2, 3, 4, 5, 6, 7, 8, 9, 10, 11, 12, 13, 14, 15 or 16 of: AR, cMET, ER, HER2, MGMT, PGP, PR, PTEN, RRM1, SPARCm, SPARCp, TLE3, TOP2A, TOPO1, TS, TUBB3. Assessing the panel of gene or gene products may comprise using sequence analysis to assess 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33 or 34 of: ABL1, AKT1, ALK, APC, ATM, BRAF, cKIT, cMET, CSF1R, CTNNB1, EGFR, ERBB2, FGFR1, FGFR2, FLT3, GNA11, GNAQ, GNAS, HRAS, IDH1, JAK2, KDR (VEGFR2), KRAS, MLH1, MPL, NOTCH1, NRAS, PDGFRA, PIK3CA, PTEN, RET, SMO, TP53, VHL. Assessing the panel of gene or gene products may comprise using ISH to assess 1 or 2, of: cMET, HER2; using IHC to assess 1, 2, 3, 4, 5, 6, 7, 8, 9, 10, 11, 12, 13, 14, 15 or 16 of: AR, cMET, ER, HER2, MGMT, PGP, PR, PTEN, RRM1, SPARCm, SPARCp, TLE3, TOP2A, TOPO1, TS, TUBB3; and/or using sequence analysis to assess 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33 or 34 of: ABL1, AKT1, ALK, APC, ATM, BRAF, cKIT, cMET, CSFIR, CTNNB1, EGFR, ERBB2, FGFR1, FGFR2, FLT3, GNA11, GNAQ, GNAS, HRAS, IDH1, JAK2, KDR (VEGFR2), KRAS, MLH1, MPL, NOTCH1, NRAS, PDGFRA, PIK3CA, PTEN, RET, SMO, TP53, VHL. Assessing the panel of gene or gene products may comprise using sequence analysis to assess 1, 2, 3, 4, 5, 6, 7, 8, 9, 10 or 11 of CDH1, ERBB4, FBXW7, HNF1A, JAK3, NPM1, PTPN11, RB1, SMAD4, SMARCB1 and STK11. In some embodiments, the sequence analysis comprises Next Generation Sequencing. In various embodiments, the sequence analysis of BRAF comprises PCR, e.g., the FDA approved cobas PCR assay.

In an aspect, the invention provides a method of identifying one or more candidate treatment for a colorectal cancer in a subject in need thereof, comprising: (a) determining a molecular profile for a sample from the subject by assessing a panel of gene or gene products, wherein the panel of gene or gene products are assessed as indicated in Table 15, FIG. 33M or FIG. 33N; and (b) identifying one or more treatment that is beneficially associated with the molecular profile of the subject, and optionally one or more treatment associated with lack of benefit, according to the determining in (a) and one or more rules in Table 16, thereby identifying the one or more candidate treatment. The panel of gene or gene products can include 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57 or 58, of: ABL1, AKT1, ALK, APC, AR, ATM, BRAF, CDH1, cKIT, cMET, CSF1R, CTNNB1, EGFR, ER, ERBB2, ERBB4, FBXW7, FGFR1, FGFR2, FLT3, GNA11, GNAQ, GNAS, HER2, HNF1A, HRAS, IDH1, JAK2, JAK3, KDR (VEGFR2), KRAS, MGMT, MLH1, MPL, NOTCH1, NPM1, NRAS, PDGFRA, PGP, PIK3CA, PR, PTEN, PTPN11, RB1, RET, RRM1, SMAD4, SMARCB1, SMO, SPARC, STK11, TLE3, TOP2A, TOPO1, TP53, TS, TUBB3, VHL. Assessing the panel of gene or gene products may comprise using ISH to assess 1 or 2 of: cMET, HER2. Assessing the panel of gene or gene products may comprise using IHC to assess 1, 2, 3, 4, 5, 6, 7, 8, 9, 10, 11, 12, 13, 14, 15 or 16 of: AR, cMET, ER, HER2, MGMT, PGP, PR, PTEN, RRM1, SPARCm, SPARCp, TLE3, TOP2A, TOPO1, TS, TUBB3. Assessing the panel of gene or gene products may comprise using sequence analysis to assess 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33 or 34 of: ABL1, AKT1, ALK, APC, ATM, BRAF, cKIT, cMET, CSFIR, CTNNB1, EGFR, ERBB2, FGFR1, FGFR2, FLT3, GNA11, GNAQ, GNAS, HRAS, IDH1, JAK2, KDR (VEGFR2), KRAS, MLH1, MPL, NOTCH1, NRAS, PDGFRA, PIK3CA, PTEN, RET, SMO, TP53, VHL. Assessing the panel of gene or gene products may comprise using ISH to assess 1 or 2 of: cMET, HER2; using IHC to assess 1, 2, 3, 4, 5, 6, 7, 8, 9, 10, 11, 12, 13, 14, 15 or 16 of: AR, cMET, ER, HER2, MGMT, PGP, PR, PTEN, RRM1, SPARCm, SPARCp, TLE3, TOP2A, TOPO1, TS, TUBB3; and/or using sequence analysis to assess 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33 or 34 of: ABL1, AKT1, ALK, APC, ATM, BRAF, cKIT, cMET, CSF1R, CTNNB1, EGFR, ERBB2, FGFR1, FGFR2, FLT3, GNA11, GNAQ, GNAS, HRAS, IDH1, JAK2, KDR (VEGFR2), KRAS, MLH1, MPL, NOTCH1, NRAS, PDGFRA, PIK3CA, PTEN, RET, SMO, TP53, VHL. Assessing the panel of gene or gene products may comprise using sequence analysis to assess 1, 2, 3, 4, 5, 6, 7, 8, 9, 10 or 11 of CDH1, ERBB4, FBXW7, HNF1A, JAK3, NPM1, PTPN11, RB1, SMAD4, SMARCB1 and STK11. In some embodiments, the sequence analysis comprises Next Generation Sequencing.

In an aspect, the invention provides a method of identifying one or more candidate treatment for a lung cancer in a subject in need thereof, comprising: (a) determining a molecular profile for a sample from the subject by assessing a panel of gene or gene products, wherein the panel of gene or gene products are assessed as indicated in Table 17, FIG. 33I or FIG. 33J; and (b) identifying one or more treatment that is beneficially associated with the molecular profile of the subject, and optionally one or more treatment associated with lack of benefit, according to the determining in (a) and one or more rules in Table 18, thereby identifying the one or more candidate treatment. The panel of gene or gene products can include 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58 or 59 of: ABL1, AKT1, ALK, APC, AR, ATM, BRAF, CDH1, cKIT, cMET, CSF1R, CTNNB1, EGFR, ER, ERBB2, ERBB4, FBXW7, FGFR1, FGFR2, FLT3, GNA11, GNAQ, GNAS, HER2, HNF1A, HRAS, IDH1, JAK2, JAK3, KDR (VEGFR2), KRAS, MGMT, MLH1, MPL, NOTCH1, NPM1, NRAS, PDGFRA, PGP, PIK3CA, PR, PTEN, PTPN11, RB1, RET, ROS1, RRM1, SMAD4, SMARCB1, SMO, SPARC, STK11, TLE3, TOP2A, TOPO1, TP53, TS, TUBB3, VHL. Assessing the panel of gene or gene products may comprise using ISH to assess 1, 2, 3 or 4, of: ALK, cMET, HER2, ROS1. Assessing the panel of gene or gene products may comprise using IHC to assess 1, 2, 3, 4, 5, 6, 7, 8, 9, 10, 11, 12, 13, 14, 15, 16 or 17 of: AR, cMET, EGFR (H-score), ER, HER2, MGMT, PGP, PR, PTEN, RRM1, SPARCm, SPARCp, TLE3, TOP2A, TOPO1, TS, TUBB3. Assessing the panel of gene or gene products may comprise using sequence analysis to assess 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33 or 34 of: ABL1, AKT1, ALK, APC, ATM, BRAF, cKIT, cMET, CSF1R, CTNNB1, EGFR, ERBB2, FGFR1, FGFR2, FLT3, GNA11, GNAQ, GNAS, HRAS, IDH1, JAK2, KDR (VEGFR2), KRAS, MLH1, MPL, NOTCH1, NRAS, PDGFRA, PIK3CA, PTEN, RET, SMO, TP53, VHL. Assessing the panel of gene or gene products may comprise using ISH to assess 1, 2, 3 or 4, of: ALK, cMET, HER2, ROS1; using IHC to assess 1, 2, 3, 4, 5, 6, 7, 8, 9, 10, 11, 12, 13, 14, 15, 16 or 17 of: AR, cMET, EGFR (H-score), ER, HER2, MGMT, PGP, PR, PTEN, RRM1, SPARCm, SPARCp, TLE3, TOP2A, TOPO1, TS, TUBB3; and/or using sequence analysis to assess 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33 or 34 of: ABL1, AKT1, ALK, APC, ATM, BRAF, cKIT, cMET, CSF1R, CTNNB1, EGFR, ERBB2, FGFR1, FGFR2, FLT3, GNA11, GNAQ, GNAS, HRAS, IDH1, JAK2, KDR (VEGFR2), KRAS, MLH1, MPL, NOTCH1, NRAS, PDGFRA, PIK3CA, PTEN, RET, SMO, TP53, VHL. Assessing the panel of gene or gene products may comprise using sequence analysis to assess 1, 2, 3, 4, 5, 6, 7, 8, 9, 10 or 11 of CDH1, ERBB4, FBXW7, HNF1A, JAK3, NPM1, PTPN11, RB1, SMAD4, SMARCB1 and STK11. In some embodiments, the sequence analysis comprises Next Generation Sequencing. The lung cancer can include without limitation a non-small cell lung cancer (NSCLC) or a bronchioloalveolar cancer (BAC).

In an aspect, the invention provides a method of identifying one or more candidate treatment for a glioma brain cancer in a subject in need thereof, comprising: (a) determining a molecular profile for a sample from the subject by assessing a panel of gene or gene products, wherein the panel of gene or gene products are assessed as indicated in Table 21, FIG. 33O or FIG. 33P; and (b) identifying one or more treatment that is beneficially associated with the molecular profile of the subject, and optionally one or more treatment associated with lack of benefit, according to the determining in (a) and one or more rules in Table 19, thereby identifying the one or more candidate treatment. The panel of gene or gene products can include 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 or 61, of: ABL1, AKT1, ALK, APC, AR, ATM, BRAF, CDH1, cKIT, cMET, CSF1R, CTNNB1, EGFR, EGFRvIII, ER, ERBB2, ERBB4, FBXW7, FGFR1, FGFR2, FLT3, GNA11, GNAQ, GNAS, HER2, HNF1A, HRAS, IDH1, IDH2, JAK2, JAK3, KDR (VEGFR2), KRAS, MGMT-Me, MLH1, MPL, NOTCH1, NPM1, NRAS, PDGFRA, PGP, PIK3CA, PR, PTEN, PTPN11, RB1, RET, RRM1, SMAD4, SMARCB1, SMO, SPARCm, SPARCp, STK11, TLE3, TOP2A, TOPO1, TP53, TS, TUBB3, VHL. Assessing the panel of gene or gene products may comprise using ISH to assess 1 or 2 of: cMET, HER2. Assessing the panel of gene or gene products may comprise using IHC to assess 1, 2, 3, 4, 5, 6, 7, 8, 9, 10, 11, 12, 13, 14 or 15, of: AR, cMET, ER, HER2, PGP, PR, PTEN, RRM1, SPARCm, SPARCp, TLE3, TOP2A, TOPO1, TS, TUBB3. Assessing the panel of gene or gene products may comprise assessing methylation of the MGMT promoter region. Assessing methylation of the MGMT promoter region can be performed using pyrosequencing and/or methylation specific PCR (MS-PCR). Assessing the panel of gene or gene products may comprise sequence analysis of IDH2. Sequence analysis of IDH2 can be performed using Sanger sequencing or Next Generation Sequencing. Assessing the panel of gene or gene products may comprise detection of the EGFRvIII variant. The EGFRvIII variant can be detected by fragment analysis. Assessing the panel of gene or gene products may comprise using sequence analysis to assess 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33 or 34 of: ABL1, AKT1, ALK, APC, ATM, BRAF, cKIT, cMET, CSF1R, CTNNB1, EGFR, ERBB2, FGFR1, FGFR2, FLT3, GNA11, GNAQ, GNAS, HRAS, IDH1, JAK2, KDR (VEGFR2), KRAS, MLH1, MPL, NOTCH1, NRAS, PDGFRA, PIK3CA, PTEN, RET, SMO, TP53, VHL. Assessing the panel of gene or gene products may comprise using ISH to assess 1 or 2 of: cMET, HER2; using IHC to assess 1, 2, 3, 4, 5, 6, 7, 8, 9, 10, 11, 12, 13, 14 or 15, of: AR, cMET, ER, HER2, PGP, PR, PTEN, RRM1, SPARCm, SPARCp, TLE3, TOP2A, TOPO1, TS, TUBB3; using pyrosequencing to detect methylation of the MGMT promoter; using Sanger sequencing to assess the sequence of IDH2; using fragment analysis to detect the EGFRvIII variant; and/or using sequence analysis to assess 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33 or 34 of: ABL1, AKT1, ALK, APC, ATM, BRAF, cKIT, cMET, CSF1R, CTNNB1, EGFR, ERBB2, FGFR1, FGFR2, FLT3, GNA11, GNAQ, GNAS, HRAS, IDH1, JAK2, KDR (VEGFR2), KRAS, MLH1, MPL, NOTCH1, NRAS, PDGFRA, PIK3CA, PTEN, RET, SMO, TP53, VHL. Assessing the panel of gene or gene products may comprise using sequence analysis to assess 1, 2, 3, 4, 5, 6, 7, 8, 9, 10 or 11 of CDH1, ERBB4, FBXW7, HNF1A, JAK3, NPM1, PTPN11, RB1, SMAD4, SMARCB1 and STK11. In some embodiments, the sequence analysis comprises Next Generation Sequencing.

In an aspect, the invention provides a method of identifying one or more candidate treatment for a gastrointestinal stromal tumor (GIST) cancer in a subject in need thereof, comprising: (a) determining a molecular profile for a sample from the subject by assessing a panel of gene or gene products, wherein the panel of gene or gene products are assessed as indicated in Table 21; and (b) identifying one or more treatment that is beneficially associated with the molecular profile of the subject, and optionally one or more treatment associated with lack of benefit, according to the determining in (a) and one or more rules in Table 20, thereby identifying the one or more candidate treatment. The panel of gene or gene products can include 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57 or 58, of: ABL1, AKT1, ALK, APC, AR, ATM, BRAF, CDH1, cKIT, cMET, CSF1R, CTNNB1, EGFR, ER, ERBB2, ERBB4, FBXW7, FGFR1, FGFR2, FLT3, GNA11, GNAQ, GNAS, HER2, HNF1A, HRAS, IDH1, JAK2, JAK3, KDR (VEGFR2), KRAS, MGMT, MLH1, MPL, NOTCH1, NPM1, NRAS, PDGFRA, PGP, PIK3CA, PR, PTEN, PTPN11, RB1, RET, RRM1, SMAD4, SMARCB1, SMO, SPARC, STK11, TLE3, TOP2A, TOPO1, TP53, TS, TUBB3, VHL. Assessing the panel of gene or gene products may comprise using ISH to assess 1 or 2 of: cMET, HER2. Assessing the panel of gene or gene products may comprise using IHC to assess 1, 2, 3, 4, 5, 6, 7, 8, 9, 10, 11, 12, 13, 14, 15 or 16 of: AR, cMET, ER, HER2, MGMT, PGP, PR, PTEN, RRM1, SPARCm, SPARCp, TLE3, TOP2A, TOPO1, TS, TUBB3. Assessing the panel of gene or gene products may comprise using sequence analysis to assess 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33 or 34 of: ABL1, AKT1, ALK, APC, ATM, BRAF, cKIT, cMET, CSF1R, CTNNB1, EGFR, ERBB2, FGFR1, FGFR2, FLT3, GNA11, GNAQ, GNAS, HRAS, IDH1, JAK2, KDR (VEGFR2), KRAS, MLH1, MPL, NOTCH1, NRAS, PDGFRA, PIK3CA, PTEN, RET, SMO, TP53, VHL. Assessing the panel of gene or gene products may comprise using ISH to assess 1 or 2 of: cMET, HER2; using IHC to assess 1, 2, 3, 4, 5, 6, 7, 8, 9, 10, 11, 12, 13, 14, 15 or 16 of: AR, cMET, ER, HER2, MGMT, PGP, PR, PTEN, RRM1, SPARCm, SPARCp, TLE3, TOP2A, TOPO1, TS, TUBB3; and/or using sequence analysis to assess 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33 or 34 of: ABL1, AKT1, ALK, APC, ATM, BRAF, cKIT, cMET, CSF1R, CTNNB1, EGFR, ERBB2, FGFR1, FGFR2, FLT3, GNA11, GNAQ, GNAS, HRAS, IDH1, JAK2, KDR (VEGFR2), KRAS, MLH1, MPL, NOTCH1, NRAS, PDGFRA, PIK3CA, PTEN, RET, SMO, TP53, VHL. Assessing the panel of gene or gene products may comprise using sequence analysis to assess 1, 2, 3, 4, 5, 6, 7, 8, 9, 10 or 11 of CDH1, ERBB4, FBXW7, HNF1A, JAK3, NPM1, PTPN11, RB1, SMAD4, SMARCB1 and STK11. In some embodiments, the sequence analysis comprises Next Generation Sequencing.

In an aspect, the invention provides a method of identifying one or more candidate treatment for a cancer in a subject in need thereof, comprising: (a) determining a molecular profile for a sample from the subject by assessing a panel of gene or gene products, wherein the panel of gene or gene products are assessed using IHC for 1, 2, 3, 4, 5, 6, 7, 8, 9, 10, 11, 12, 13, 14, 15, 16 or 17 of AR, cMET, EGFR (including H-score for NSCLC), ER, HER2, MGMT, PGP, PR, PTEN, RRM1, SPARCm, SPARCp, TLE3, TOPO1, TOP2A, TS, TUBB3; FISH or CISH for 1, 2, 3, 4, or 5 of ALK, cMET, HER2, ROS1, TOP2A; Mutational Analysis of 1, 2, 3 or 4 of BRAF (e.g., Cobas® PCR), IDH2 (e.g., Sanger Sequencing), MGMT promoter methylation (e.g., by PyroSequencing), EGFR (e.g., fragment analysis to detect EGFRvIII); and/or Mutational Analysis (e.g., by Next-Generation Sequencing) of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 of ABL1, AKT1, ALK, APC, ATM, BRAF, CDH1, CSF1R, CTNNB1, EGFR, ERBB2 (HER2), ERBB4, FBXW7, FGFR1, FGFR2, FLT3, GNA11, GNAQ, GNAS, I-INF1A, HRAS, IDH1, JAK2, JAK3, KDR (VEGFR2), KIT, KRAS, MET, MLH1, MPL, NOTCH1, NPM1, NRAS, PDGFRA, PIK3CA, PTEN, PTPN11, RB1, RET, SMAD4, SMARCB1, SMO, STK11, TP53, VHL; and (b) identifying one or more treatment that is beneficially associated with the molecular profile of the subject, and optionally one or more treatment associated with lack of benefit, according to the determining in (a) and one or more rules in any of Tables 7-22, thereby identifying the one or more candidate treatment.

In the method of identifying one or more candidate treatment provided by the invention, the methods may further comprising additional molecular profiling according to FIG. 33Q.

In an aspect, the invention provides a method of identifying one or more candidate treatment for a prostate cancer in a subject in need thereof, comprising: (a) determining a molecular profile for a sample from the subject on a panel of gene or gene products, wherein the panel of gene or gene products comprises immunohistochemistry (IHC) of AR, MRP1, TOPO1, TLE3, EGFR, TS, PGP, TUBB3, RRM1, PTEN and/or MGMT; in situ hybridization (ISH) of EGFR and/or cMYC; and/or sequencing of TP53, PTEN, CTNNB1, PIK3CA, RB1, ATM, cMET, K/HRAS, ERBB4, ALK, BRAF and/or cKIT; and (b) identifying one or more treatment that is beneficially associated with the molecular profile of the subject, and optionally one or more treatment associated with lack of benefit, according to the determining in (a) and one or more rules in Table 22, thereby identifying the one or more candidate treatment. The rules can include one or more of: imatinib for patients with high cKIT or PDGFRA; cetuximab for patients with EGFR positivity; cabozantinib for patients with cMET aberrations; PAM pathway inhibitors (e.g., BEZ234, everolimus) for patients with PIK3CA pathway activation; HDAC inhibitors for patients with cMYC amplification; 5-FU for patients with low TS; gemcitabine for patients with low RRM1; temozolomide for patients with low MGMT; cabazitaxel for patients with low TUBB3 or PGP, or high TLE3; and anti-androgen agents (e.g., enzalutamide) for patients with high AR.

In an aspect, the invention provides a method of identifying one or more candidate treatment for a cancer in a subject in need thereof, comprising: a) determining a molecular profile for a sample from the subject by sequencing a panel of gene or gene products, wherein the panel of gene or gene products comprises one or more gene in Table 24; and b) identifying one or more treatment that is beneficially associated with the molecular profile of the subject, and optionally one or more treatment associated with lack of benefit, according to the determining in (a) and one or more rules in Table 25 or any of Tables 7-22, thereby identifying the one or more candidate treatment. Assessing the panel of gene or gene products may comprise using sequence analysis to assess 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 of ABL1, AKT1, ALK, APC, ATM, BRAF, CDH1, CSF1R, CTNNB1, EGFR, ERBB2 (HER2), ERBB4, FBXW7, FGFR1, FGFR2, FLT3, GNA11, GNAQ, GNAS, HNF1A, HRAS, IDH1, JAK2, JAK3, KDR (VEGFR2), KIT, KRAS, MET, MLH1, MPL, NOTCH1, NPM1, NRAS, PDGFRA, PIK3CA, PTEN, PTPN11, RB1, RET, SMAD4, SMARCB1, SMO, STK11, TP53, VHL. Assessing the panel of gene or gene products may comprise using sequence analysis to assess 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33 or 34 of: ABL1, AKT1, ALK, APC, ATM, BRAF, cKIT, cMET, CSF1R, CTNNB1, EGFR, ERBB2, FGFR1, FGFR2, FLT3, GNA11, GNAQ, GNAS, HRAS, IDH1, JAK2, KDR (VEGFR2), KRAS, MLH1, MPL, NOTCH1, NRAS, PDGFRA, PIK3CA, PTEN, RET, SMO, TP53, VHL. Assessing the panel of gene or gene products may comprise using sequence analysis to assess 1, 2, 3, 4, 5, 6, 7, 8, 9, 10, 11, 12, 13, 14 or 15 of ABL1, APC, BRAF, EGFR, FLT3, GNAQ, IDH1, JAK2, cKIT, KRAS, MPL, NPM1, NRAS, PDGFRA, VHL. Assessing the panel of gene or gene products may comprise using sequence analysis to assess 1, 2, 3, 4, 5, 6, 7, 8, 9, 10, 11, 12, 13 or 14 of ABL1, APC, BRAF, EGFR, FLT3, GNAQ, IDH1, JAK2, cKIT, KRAS, MPL, NRAS, PDGFRA, VHL.

In the methods of the invention above, identifying the one or more treatment that is beneficially associated with the molecular profile of the subject, and optionally the one or more treatment associated with lack of benefit, can comprise: a) correlating the molecular profile with the one or more rules, wherein the one or more rules comprise a mapping of treatments whose efficacy has been previously determined in individuals having cancers that have different levels of, overexpress, underexpress, and/or have mutations in one or more members of the panel of gene or gene products; and b) identifying one or more treatment that is associated with treatment benefit based on the correlating in (a); and c) optionally identifying one or more treatment that is associated with lack of treatment benefit based on the correlating in (a). The mapping of treatments can be any of those included in Tables 3-5, 7-23, FIGS. 33A-Q, FIGS. 35A-I, or FIGS. 36A-F.

The methods of the invention above may further comprise identifying one or more candidate clinical trial for the subject based on the molecular profiling.

In an aspect, the invention provides a method of identifying one or more candidate clinical trial for a subject having a cancer, comprising: (a) determining a molecular profile for a sample from the subject on a panel of gene or gene products; and (b) identifying one or more clinical trial associated with the molecular profile of the subject according to the determining in (a) and one or more biomarker-clinical trial association rules, thereby identifying the one or more candidate clinical trial. The molecular profile can include IHC for 1, 2, 3, 4, 5, 6, 7, 8, 9, 10, 11, 12, 13, 14, 15, 16 or 17 of AR, cMET, EGFR (including H-score for NSCLC), ER, HER2, MGMT, Pgp, PR, PTEN, RRM1, SPARCm, SPARCp, TLE3, TOPO1, TOP2A, TS, TUBB3; FISH or CISH for 1, 2, 3, 4, or 5 of ALK, cMET, HER2, ROS1, TOP2A; Mutational Analysis of 1, 2, 3 or 4 of BRAF (e.g., Cobas® PCR), IDH2 (e.g., Sanger Sequencing), MGMT promoter methylation (e.g., by PyroSequencing), EGFR (e.g., fragment analysis to detect EGFRvIII); and/or Mutational Analysis (e.g., by Next-Generation Sequencing) of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 of ABL1, AKT1, ALK, APC, ATM, BRAF, CDH1, CSF1R, CTNNB1, EGFR, ERBB2 (HER2), ERBB4, FBXW7, FGFR1, FGFR2, FLT3, GNA11, GNAQ, GNAS, HNF1A, HRAS, IDH1, JAK2, JAK3, KDR (VEGFR2), KIT, KRAS, MET, MLH1, MPL, NOTCH1, NPM1, NRAS, PDGFRA, PIK3CA, PTEN, PTPN11, RB1, RET, SMAD4, SMARCB1, SMO, STK11, TP53, VHL.

Identifying the one or more clinical trial associated with the molecular profile of the subject according to the methods above can comprise: 1) matching to clinical trials for non-standard of care treatments for the patient's cancer (e.g., off NCCN compendium treatments) indicated as potentially beneficial according to the biomarker—drug association rules herein; 2) matching to clinical trials based on biomarker eligibility requirements of the trial; and/or 3) matching to clinical trials based on the molecular profile of the patient, biology of the disease and/or associated signaling pathways. In some embodiments, matching to clinical trials based on the molecular profile of the patient, biology of the disease and/or associated signaling pathways comprises: 1) matching trials with therapeutic agents directly targeting a gene and/or gene product in the molecular profile; 2) matching trials with therapeutic agents that target another gene or gene product in a biological pathway that directly target a gene and/or gene product in the molecular profile; 3) matching trials with therapeutic agents that target another gene or gene product in a biological pathway that indirectly target a gene and/or gene product in the molecular profile. Identifying the one or more candidate clinical trial can be performed according to one or more biomarker-clinical trial association rules in Tables 28-29.

As desired, additional genes and/or gene products may be assessed according to the methods of the invention. For example, the molecular profiles above may comprise one or more additional gene or gene product listed in Table 2, Table 6 or Table 25. Additional genes and/or gene products can be assessed as evidence becomes available linking such genes and/or gene products to a therapeutic efficacy. The one or more additional gene or gene product listed in Table 2, Table 6 or Table 25 can be assessed by any appropriate laboratory technique such as described herein, including without limitation next generation sequencing.

The sample used to perform molecular profiling in the methods of the invention can include one or more of a formalin-fixed paraffin-embedded (FFPE) tissue, fixed tissue, core needle biopsy, fine needle aspirate, unstained slides, fresh frozen (FF) tissue, formalin samples, tissue comprised in a solution that preserves nucleic acid or protein molecules, and/or a bodily fluid sample. In some embodiments, the sample comprises cells from a solid tumor. In some embodiments, the sample comprises a bodily fluid. The bodily fluid can be a malignant fluid. The bodily fluid can be a pleural or peritoneal fluid. In various embodiments, the bodily fluid comprises peripheral blood, sera, plasma, ascites, urine, cerebrospinal fluid (CSF), sputum, saliva, bone marrow, synovial fluid, aqueous humor, amniotic fluid, cerumen, breast milk, broncheoalveolar lavage fluid, semen, prostatic fluid, cowper's fluid or pre-ejaculatory fluid, female ejaculate, sweat, fecal matter, hair, tears, cyst fluid, pleural and peritoneal fluid, pericardial fluid, lymph, chyme, chyle, bile, interstitial fluid, menses, pus, sebum, vomit, vaginal secretions, mucosal secretion, stool water, pancreatic juice, lavage fluids from sinus cavities, bronchopulmonary aspirates, blastocyl cavity fluid, or umbilical cord blood. The sample may comprise a microvesicle population. In such cases, one or more members of the panel of gene or gene products may be associated with the microvesicle population.

The one or more candidate treatment can be selected from those listed in any of Tables 3-5, 7-22, 28, 29, 33, 36 or 37 herein. The methods of the invention may provide a prioritized list of one or more candidate treatment.

The cancer that is profiled according to the methods of the invention can be of any stage or progression. In some embodiments, the subject has not previously been treated with the one or more candidate treatment associated with treatment benefit. In some embodiments, the cancer comprises a metastatic cancer. In some embodiments, the cancer comprises a recurrent cancer. In some embodiments, the cancer is refractory to a prior treatment. The prior treatment can be the standard of care for the cancer, e.g., as based on the available evidence and/or guidelines such as the NCCN compendium. The cancer may be refractory to all known standard of care treatments. Alternately, the subject has not previously been treated for the cancer.

The one or more candidate treatment can be administered to the subject. In some embodiments of the methods herein, progression free survival (PFS) or disease free survival (DFS) for the subject is extended by administration of the one or more candidate treatment to the subject. The subject's lifespan can be extended by administration of the one or more candidate treatment to the subject.

In the methods of the invention above, the molecular profile can be compared to the one or more rules using a computer. The one or more rules may be comprised within a computer database.

In another aspect, the invention provides a method of generating a molecular profiling report comprising preparing a report comprising results of the molecular profile determined by any of the methods of the invention, e.g., as described above. Illustrative reports are shown in FIGS. 37A-37Y, FIGS. 38A-38AA and FIGS. 39A-39Y. In some embodiments, the report further comprises a list of the one or more candidate treatment that is associated with benefit for treating the cancer. The report may further comprise identification of the one or more candidate treatment as standard of care or not for the cancer lineage. The report can also comprise a list of one or more treatment that is associated with lack of benefit for treating the cancer. The report can also comprise a list of one or more treatment that is associated with indeterminate benefit for treating the cancer. In some embodiments, the report comprises a listing of members of the panel of genes or gene products assessed with description of each. In some embodiments, the report comprises a listing of members of the panel of genes or gene products assessed by one or more of ISH, IHC, Next Generation sequencing, Sanger sequencing, PCR, pyrosequencing and fragment analysis. In some embodiments, the report comprises a list of clinical trials for which the subject is eligible based on the molecular profile. In some embodiments, the report comprises a list of evidence supporting the identification of certain treatments as likely to benefit the patient, not benefit the patient, or having indeterminate benefit. The report may comprise: 1) a list of the genes and/or gene products in the molecular profile; 2) a description of the molecular profile of the genes and/or gene products as determined for the subject; 3) a treatment associated with one or more of the genes and/or gene products in the molecular profile; and 4) and an indication whether each treatment is likely to benefit the patient, not benefit the patient, or has indeterminate benefit. The description of the molecular profile of the genes and/or gene products as determined for the subject can comprise the technique used to assess the gene and/or gene products and the results of the assessment.

In an aspect, the invention provides a method of generating a molecular profiling report comprising preparing a report comprising results of the molecular profile determined by the methods for identifying one or more candidate clinical trial as provided herein, e.g., as provided above. The report can include a list of the one or more identified candidate clinical trial.

The molecular profile reports of the invention can be computer generated reports. Such reports may be provided as a printed report and/or as a computer file. The molecular profile report can be made accessible via a web portal. The reports can be transmitted over a network. In some embodiments, the results of some or all of the molecular profiling are transmitted over a network before the report is compiled.

In an aspect, the invention contemplates use of a reagent in carrying out the methods of the invention. In a related aspect, the invention contemplates use of a reagent in the manufacture of a reagent or kit for carrying out the method of the invention. In still another related aspect, the invention provides a kit comprising a reagent for carrying out the method of the invention. The reagent can be any reagent useful for carrying out one or more of the molecular profiling methods provided herein. For example, the reagent can include without limitation one or more of a reagent for extracting nucleic acid from a sample, a reagent for performing ISH, a reagent for performing IHC, a reagent for performing PCR, a reagent for performing Sanger sequencing, a reagent for performing next generation sequencing, a reagent for a DNA microarray, a reagent for performing pyrosequencing, a nucleic acid probe, a nucleic acid primer, an antibody, a reagent for performing bisulfite treatment of nucleic acid.

In a related aspect, the invention provides a report generated by the methods of report generation as described herein, e.g., as described above. Illustrative reports are shown in FIGS. 37A-37Y, FIGS. 38A-38AA and FIGS. 39A-39Y.

In an aspect, the invention provides a computer system for generating the report provided by the invention.

In a related aspect, the invention provides a system for identifying one or more candidate treatment for a cancer comprising: a host server; a user interface for accessing the host server to access and input data; a processor for processing the inputted data; a memory coupled to the processor for storing the processed data and instructions for: i) accessing a molecular profile generated by the method of the invention, e.g., as described above; ii) identifying one or more candidate treatment that is associated with likely treatment benefit by comparing the molecular profiling results to the one or more rules; iii) optionally identifying one or more treatment that is associated with likely lack of treatment benefit by comparing the molecular profiling results to the one or more rules; and iv) optionally identifying one or more treatment that is associated with indeterminate treatment benefit by comparing the molecular profiling results to the one or more rules; and a display for displaying the identified one or more candidate treatment that is associated with likely treatment benefit and the optional one or more treatment that is associated with likely lack of treatment benefit and one or more treatment that is associated with indeterminate treatment benefit. The display may comprise a report as described above. The systems of the invention may further comprise instructions for identifying one or more clinical trial that is associated with likely treatment benefit by comparing the molecular profiling results to one or more biomarker-clinical trial association rules.

In an aspect, the invention provides a system for identifying one or more candidate clinical trial for a cancer comprising: a host server; a user interface for accessing the host server to access and input data; a processor for processing the inputted data; a memory coupled to the processor for storing the processed data and instructions for: accessing a molecular profile generated by the methods of identifying one or more candidate clinical trial provided by the invention; and identifying one or more candidate candidate clinical trial by comparing the molecular profiling results to the one or more rules; and a display for displaying the identified one or more candidate candidate clinical trial. The display may comprise a report as described above.

In an aspect, the invention provides a computer medium comprising one or more rules from any of Tables 7, 9, 11, 13, 15, 17, 21 and 28. In an embodiment, the computer medium comprises one or more rules selected from: performing IHC on RRM1 to determine likely benefit or lack of benefit from an antimetabolite and/or gemcitabine; performing IHC on TS to determine likely benefit or lack of benefit from a TOPO1 inhibitor, irinotecan and/or topotecan; performing IHC on TS to determine likely benefit or lack of benefit from an antimetabolite, fluorouracil, capecitabine, and/or pemetrexed; performing IHC on MGMT to determine likely benefit or lack of benefit from an alkylating agent, temozolomide, and/or dacarbazine; performing IHC on AR to determine likely benefit or lack of benefit from an anti-androgen, bicalutamide, flutamide, and/or abiraterone; performing IHC on ER to determine likely benefit or lack of benefit from a hormonal agent, tamoxifen, fulvestrant, letrozole, and/or anastrozole; performing IHC on one or more of ER and PR to determine likely benefit or lack of benefit from a hormonal agent, tamoxifen, toremifene, fulvestrant, letrozole, anastrozole, exemestane, megestrol acetate, leuprolide, and/or goserelin; performing one or more of IHC on HER2 and ISH on HER2 to determine likely benefit or lack of benefit from a tyrosine kinase inhibitor and/or lapatinib; performing one or more of IHC on HER2 and ISH on HER2 to determine likely benefit or lack of benefit from an antibody therapy, trastuzumab, pertuzumab, and/or ado-trastuzumab emtansine (T-DM1); performing one or more of ISH on TOP2A, ISH on HER2, IHC on TOP2A and IHC on PGP to determine likely benefit or lack of benefit from an anthracyclines, doxorubicin, liposomal-doxorubicin, and/or epirubicin; performing sequencing on one or more of cKIT and PDGFRA to determine likely benefit or lack of benefit from a tyrosine kinase inhibitor and/or imatinib; performing one or more of ISH on ALK and ISH on ROS1 to determine likely benefit or lack of benefit from a tyrosine kinase inhibitor and/or crizotinib; performing sequencing on PIK3CA to determine likely benefit or lack of benefit from an mTOR inhibitor, everolimus, and/or temsirolimus; performing sequencing on RET to determine likely benefit or lack of benefit from a tyrosine kinase inhibitor, and/or vandetanib; performing IHC on one or more of SPARC, TUBB3 and PGP to determine likely benefit or lack of benefit from a taxane, paclitaxel, docetaxel, nab-paclitaxel; performing IHC on one or more of SPARC, TLE3, TUBB3 and PGP to determine likely benefit or lack of benefit from a taxane, paclitaxel, docetaxel, nab-paclitaxel; performing one or more of PCR and sequencing on BRAF to determine likely benefit or lack of benefit from a tyrosine kinase inhibitor, vemurafenib, dabrafenib, and/or trametinib; performing one or more of sequencing on KRAS, sequencing on BRAF, sequencing on NRAS, sequencing on PIK3CA and IHC on PTEN to determine likely benefit or lack of benefit from an EGFR-targeted antibody, cetuximab, and/or panitumumab; performing one or more of sequencing on EGFR, sequencing on KRAS, ISH on cMET, sequencing on PIK3CA and IHC onn PTEN to determine likely benefit or lack of benefit from a tyrosine kinase inhibitor, erlotinib, and/or gefitinib; performing sequencing on EGFR to determine likely benefit or lack of benefit from a tyrosine kinase inhibitor, and/or afatinib; and performing sequencing on cKIT to determine likely benefit or lack of benefit from a tyrosine kinase inhibitor, and/or sunitinib. The computer medium can comprise one or more rules selected from Table 28. The computer medium may comprise a partial set of rules provided in any of Tables 7, 9, 11, 13, 15, 17, 21 and 28. The computer medium may comprise the full set of rules provided in any of Tables 7, 9, 11, 13, 15, 17, 21 and 28.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

A better understanding of the features and advantages of the present invention will be obtained by reference to the following detailed description that sets forth illustrative embodiments, in which the principles of the invention are used, and the accompanying drawings of which:

FIG. 1 illustrates a block diagram of an exemplary embodiment of a system for determining individualized medical intervention for a particular disease state that utilizes molecular profiling of a patient's biological specimen that is non disease specific.

FIG. 2 is a flowchart of an exemplary embodiment of a method for determining individualized medical intervention for a particular disease state that utilizes molecular profiling of a patient's biological specimen that is non disease specific.

FIGS. 3A through 3D illustrate an exemplary patient profile report in accordance with step 80 of FIG. 2.

FIG. 4 is a flowchart of an exemplary embodiment of a method for identifying a drug therapy/agent capable of interacting with a target.

FIGS. 5-14 are flowcharts and diagrams illustrating various parts of an information-based personalized medicine drug discovery system and method in accordance with the present invention.

FIGS. 15-25 are computer screen print outs associated with various parts of the information-based personalized medicine drug discovery system and method shown in FIGS. 5-14.

FIGS. 26-31 herein are incorporated by reference from FIGS. 26-31, respectively, from International Patent Application PCT/US2009/060630, filed 14 Oct. 2009 and entitled “GENE AND GENE EXPRESSED PROTEIN TARGETS DEPICTING BIOMARKER PATTERNS AND SIGNATURE SETS BY TUMOR TYPE,” which application is hereby incorporated by reference in its entirety.

FIGS. 32A-B illustrate a diagram showing a biomarker centric (FIG. 32A) and therapeutic centric (FIG. 32B) approach to identifying a therapeutic agent.

FIGS. 33A-33Q illustrate molecular intelligence (MI) profiles comprising biomarkers and associated therapeutic agents that can be assessed to identify candidate therapeutic agents. The indicated MI Plus profiles include additional cancer markers to be assessed by mutational analysis for diagnostic, prognostic and related purposes. NextGen refers to Next Generation Sequencing. PyroSeq refers to pyrosequencing. SangerSeq refers to Sanger dye termination sequencing. FIG. 33A and FIG. 33B illustrate an MI profile and and MI PLUS profile, respectively, for any solid tumor. FIG. 33C and FIG. 33D illustrate an MI profile and and MI PLUS profile, respectively, for an ovarian cancer. FIG. 33E and FIG. 33F illustrate an MI profile and and MI PLUS profile, respectively, for a melanoma. FIG. 33G and FIG. 33H illustrate an MI profile and and MI PLUS profile, respectively, for a uveal melanoma.

FIG. 33I and FIG. 33J illustrate an MI profile and and MI PLUS profile, respectively, for a non-small cell lung cancer (NSCLC). FIG. 33K and FIG. 33L illustrate an MI profile and and MI PLUS profile, respectively, for a breast cancer. FIG. 33M and FIG. 33N illustrate an MI profile and and MI PLUS profile, respectively, for a colorectal cancer (CRC). FIG. 33O and FIG. 33P illustrate an MI profile and and MI PLUS profile, respectively, for a glioma. FIG. 33Q illustrates individual marker profiling that can be added to any of the molecular profiles in FIGS. 33A-33P.

FIGS. 34A-34C illustrate biomarkers assessed using a molecular profiling approach as outlined in FIGS. 33A-33Q, Tables 7-24, and accompanying text herein. FIG. 34A illustrates biomarkers that are assessed. The biomarkers that are assessed according to the Next Generation sequencing panel in FIG. 34A are shown in FIG. 34B. FIG. 34C illustrates sample requirements that can be used to perform molecular profiling on a patient tumor sample according to the panels in FIGS. 34A-34B.

FIGS. 35A-351 illustrate biomarkers and associated therapeutic agents that can be assessed to identify candidate therapeutic agents. NextGen refers to Next Generation Sequencing.

FIGS. 36A-F illustrate how molecular profiles for any cancer, e.g., for assessment of solid tumors, can be altered depending on sample availability. FIG. 36A illustrates a core comprehensive molecular profile for cancer. FIG. 36B illustrates lineage specific components of the comprehensive molecular profile for cancer. FIG. 36C illustrates drugs and clinical trials corresponding to the profiling shown in FIGS. 36A-B. FIG. 36D illustrates a comprehensive molecular profile that can be used instead of the profile shown in FIGS. 36A-B when insufficient sample is present to perform RT-PCR. FIG. 36E illustrates additional molecular profiling that can be performed. For example, TOP2A IHC and PGP IHC can be used instead of TOP2A FISH when the sample is insufficient for FISH testing. FIG. 36F provides illustrative biomarker tests that can be prioritized for various lineages, e.g., when insufficient sample is available for comprehensive molecular profiling.

FIGS. 37A-37Y illustrate an exemplary patient report based on molecular profiling for a patient having a history of anaplastic astrocytoma, a WHO grade III type of astrocytoma, a high grade glioma.

FIGS. 38A-38AA illustrate an exemplary patient report based on molecular intelligence molecular profiling for a patient having a history of lung adenocarcinoma.

FIGS. 39A-39Y illustrate an exemplary patient report based on molecular profiling for a non-small cell lung cancer with stand alone mutational analysis.

FIG. 40 illustrates progression free survival (PFS) using therapy selected by molecular profiling (period B) with PFS for the most recent therapy on which the patient has just progressed (period A). If PFS(B)/PFS(A) ratio ≥1.3, then molecular profiling selected therapy was defined as having benefit for patient.

FIG. 41 is a schematic of methods for identifying treatments by molecular profiling if a target is identified.

FIG. 42 illustrates the distribution of the patients in the study as performed in Example 1.

FIG. 43 is graph depicting the results of the study with patients having PFS ratio ≥1.3 was 18/66 (27%).

FIG. 44 is a waterfall plot of all the patients for maximum % change of summed diameters of target lesions with respect to baseline diameter.

FIG. 45 illustrates the relationship between what clinician selected as what she/he would use to treat the patient before knowing what the molecular profiling results suggested. There were no matches for the 18 patients with PFS ratio ≥1.3.

FIG. 46 is a schematic of the overall survival for the 18 patients with PFS ratio ≥1.3 versus all 66 patients.

FIG. 47 illustrates a molecular profiling system that performs analysis of a cancer sample using a variety of components that measure expression levels, chromosomal aberrations and mutations. The molecular “blueprint” of the cancer is used to generate a prioritized ranking of druggable targets and/or drug associated targets in tumor and their associated therapies.

FIG. 48 shows an example output of microarray profiling results and calls made using a cutoff value.

FIGS. 49A-B illustrate a workflow chart for identifying a therapeutic for an individual having breast cancer. The workflow of FIG. 49A feeds into the workflow of FIG. 49B as indicated.

FIG. 50 illustrates biomarkers used for identifying a therapeutic for an individual having breast cancer such as when following the workflow of FIGS. 49A-B. The figure illustrates a biomarker centric view of the workflow described above in different cancer settings.

FIG. 51 illustrates the percentage of HER2 positive breast cancers that are likely to respond to treatment with trastuzumab (Herceptin®), which is about 30%. Characteristics of the tumor that can be identified by molecular profiling are shown as well.

### DETAILED DESCRIPTION OF THE INVENTION

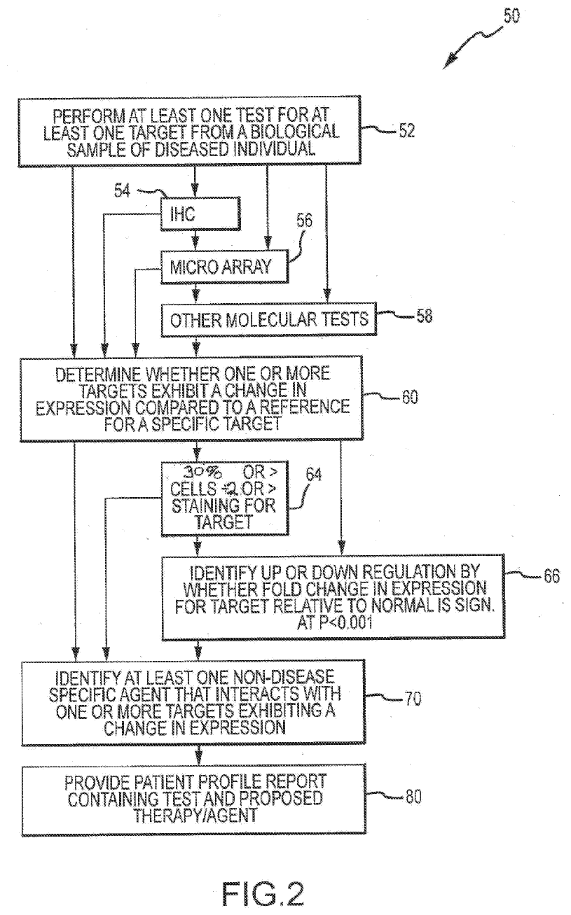

The present invention provides methods and systems for identifying therapeutic agents for use in treatments on an individualized basis by using molecular profiling. The molecular profiling approach provides a method for selecting a candidate treatment for an individual that could favorably change the clinical course for the individual with a condition or disease, such as cancer. The molecular profiling approach provides clinical benefit for individuals, such as identifying drug target(s) that provide a longer progression free survival (PFS), longer disease free survival (DFS), longer overall survival (OS) or extended lifespan. Methods and systems of the invention are directed to molecular profiling of cancer on an individual basis that can provide alternatives for treatment that may be convention or alternative to conventional treatment regimens. For example, alternative treatment regimes can be selected through molecular profiling methods of the invention where, a disease is refractory to current therapies, e.g., after a cancer has developed resistance to a standard-of-care treatment. Illustrative schemes for using molecular profiling to identify a treatment regime are shown in FIGS. 2, 49A-B and 50, each of which is described in further detail herein. Thus, molecular profiling provides a personalized approach to selecting candidate treatments that are likely to benefit a cancer. In embodiments, the molecular profiling method is used to identify therapies for patients with poor prognosis, such as those with metastatic disease or those whose cancer has progressed on standard front line therapies, or whose cancer has progressed on multiple chemotherapeutic or hormonal regimens.

Personalized medicine based on pharmacogenetic insights, such as those provided by molecular profiling according to the invention, is increasingly taken for granted by some practitioners and the lay press, but forms the basis of hope for improved cancer therapy. However, molecular profiling as taught herein represents a fundamental departure from the traditional approach to oncologic therapy where for the most part, patients are grouped together and treated with approaches that are based on findings from light microscopy and disease stage. Traditionally, differential response to a particular therapeutic strategy has only been determined after the treatment was given, i.e. a posteriori. The “standard” approach to disease treatment relies on what is generally true about a given cancer diagnosis and treatment response has been vetted by randomized phase III clinical trials and forms the “standard of care” in medical practice. The results of these trials have been codified in consensus statements by guidelines organizations such as the National Comprehensive Cancer Network and The American Society of Clinical Oncology. The NCCN Compendium™ contains authoritative, scientifically derived information designed to support decision-making about the appropriate use of drugs and biologics in patients with cancer. The NCCN Compendium™ is recognized by the Centers for Medicare and Medicaid Services (CMS) and United Healthcare as an authoritative reference for oncology coverage policy. On-compendium treatments are those recommended by such guides. The biostatistical methods used to validate the results of clinical trials rely on minimizing differences between patients, and are based on declaring the likelihood of error that one approach is better than another for a patient group defined only by light microscopy and stage, not by individual differences in tumors. The molecular profiling methods of the invention exploit such individual differences. The methods can provide candidate treatments that can be then selected by a physician for treating a patient. In a study of such an approach presented in Example 1 herein, the results were profound: in 66 consecutive patients, the treating oncologist never managed to identify the molecular target selected by the test, and 27% of patients whose treatment was guided by molecular profiling managed a remission 1.3× longer than their previous best response. At present, such results are virtually unheard of result in the salvage therapy setting.

Molecular profiling can be used to provide a comprehensive view of the biological state of a sample. In an embodiment, molecular profiling is used for whole tumor profiling. Accordingly, a number of molecular approaches are used to assess the state of a tumor. The whole tumor profiling can be used for selecting a candidate treatment for a tumor. Molecular profiling can be used to select candidate therapeutics on any sample for any stage of a disease. In embodiment, the methods of the invention are used to profile a newly diagnosed cancer. The candidate treatments indicated by the molecular profiling can be used to select a therapy for treating the newly diagnosed cancer. In other embodiments, the methods of the invention are used to profile a cancer that has already been treated, e.g., with one or more standard-of-care therapy. In embodiments, the cancer is refractory to the prior treatment/s. For example, the cancer may be refractory to the standard of care treatments for the cancer. The cancer can be a metastatic cancer or other recurrent cancer. The treatments can be on-compendium or off-compendium treatments.

Molecular profiling can be performed by any known means for detecting a molecule in a biological sample. Molecular profiling comprises methods that include but are not limited to, nucleic acid sequencing, such as a DNA sequencing or mRNA sequencing; immunohistochemistry (IHC); in situ hybridization (ISH); fluorescent in situ hybridization (FISH); chromogenic in situ hybridization (CISH); PCR amplification (e.g., qPCR or RT-PCR); various types of microarray (mRNA expression arrays, low density arrays, protein arrays, etc); various types of sequencing (Sanger, pyrosequencing, etc); comparative genomic hybridization (CGH); NextGen sequencing; Northern blot; Southern blot; immunoassay; and any other appropriate technique to assay the presence or quantity of a biological molecule of interest. In various embodiments of the invention, any one or more of these methods can be used concurrently or subsequent to each other for assessing target genes disclosed herein.

Molecular profiling of individual samples is used to select one or more candidate treatments for a disorder in a subject, e.g., by identifying targets for drugs that may be effective for a given cancer. For example, the candidate treatment can be a treatment known to have an effect on cells that differentially express genes as identified by molecular profiling techniques, an experimental drug, a government or regulatory approved drug or any combination of such drugs, which may have been studied and approved for a particular indication that is the same as or different from the indication of the subject from whom a biological sample is obtain and molecularly profiled.

When multiple biomarker targets are revealed by assessing target genes by molecular profiling, one or more decision rules can be put in place to prioritize the selection of certain therapeutic agent for treatment of an individual on a personalized basis. Rules of the invention aide prioritizing treatment, e.g., direct results of molecular profiling, anticipated efficacy of therapeutic agent, prior history with the same or other treatments, expected side effects, availability of therapeutic agent, cost of therapeutic agent, drug-drug interactions, and other factors considered by a treating physician. Based on the recommended and prioritized therapeutic agent targets, a physician can decide on the course of treatment for a particular individual. Accordingly, molecular profiling methods and systems of the invention can select candidate treatments based on individual characteristics of diseased cells, e.g., tumor cells, and other personalized factors in a subject in need of treatment, as opposed to relying on a traditional one-size fits all approach that is conventionally used to treat individuals suffering from a disease, especially cancer. In some cases, the recommended treatments are those not typically used to treat the disease or disorder inflicting the subject. In some cases, the recommended treatments are used after standard-of-care therapies are no longer providing adequate efficacy.

The treating physician can use the results of the molecular profiling methods to optimize a treatment regimen for a patient. The candidate treatment identified by the methods of the invention can be used to treat a patient; however, such treatment is not required of the methods. Indeed, the analysis of molecular profiling results and identification of candidate treatments based on those results can be automated and does not require physician involvement.

### Biological Entities

Nucleic acids include deoxyribonucleotides or ribonucleotides and polymers thereof in either single- or double-stranded form, or complements thereof. Nucleic acids can contain known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs). Nucleic acid sequence can encompass conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); Rossolini et al., Mol. Cell Probes 8:91-98 (1994)). The term nucleic acid can be used interchangeably with gene, cDNA, mRNA, oligonucleotide, and polynucleotide.

A particular nucleic acid sequence may implicitly encompass the particular sequence and “splice variants” and nucleic acid sequences encoding truncated forms. Similarly, a particular protein encoded by a nucleic acid can encompass any protein encoded by a splice variant or truncated form of that nucleic acid. “Splice variants,” as the name suggests, are products of alternative splicing of a gene. After transcription, an initial nucleic acid transcript may be spliced such that different (alternate) nucleic acid splice products encode different polypeptides. Mechanisms for the production of splice variants vary, but include alternate splicing of exons. Alternate polypeptides derived from the same nucleic acid by read-through transcription are also encompassed by this definition. Any products of a splicing reaction, including recombinant forms of the splice products, are included in this definition. Nucleic acids can be truncated at the 5′ end or at the 3′ end. Polypeptides can be truncated at the N-terminal end or the C-terminal end. Truncated versions of nucleic acid or polypeptide sequences can be naturally occurring or created using recombinant techniques.

The terms “genetic variant” and “nucleotide variant” are used herein interchangeably to refer to changes or alterations to the reference human gene or cDNA sequence at a particular locus, including, but not limited to, nucleotide base deletions, insertions, inversions, and substitutions in the coding and non-coding regions. Deletions may be of a single nucleotide base, a portion or a region of the nucleotide sequence of the gene, or of the entire gene sequence. Insertions may be of one or more nucleotide bases. The genetic variant or nucleotide variant may occur in transcriptional regulatory regions, untranslated regions of mRNA, exons, introns, exon/intron junctions, etc. The genetic variant or nucleotide variant can potentially result in stop codons, frame shifts, deletions of amino acids, altered gene transcript splice forms or altered amino acid sequence.

An allele or gene allele comprises generally a naturally occurring gene having a reference sequence or a gene containing a specific nucleotide variant.

A haplotype refers to a combination of genetic (nucleotide) variants in a region of an mRNA or a genomic DNA on a chromosome found in an individual. Thus, a haplotype includes a number of genetically linked polymorphic variants which are typically inherited together as a unit.

As used herein, the term “amino acid variant” is used to refer to an amino acid change to a reference human protein sequence resulting from genetic variants or nucleotide variants to the reference human gene encoding the reference protein. The term “amino acid variant” is intended to encompass not only single amino acid substitutions, but also amino acid deletions, insertions, and other significant changes of amino acid sequence in the reference protein.

The term “genotype” as used herein means the nucleotide characters at a particular nucleotide variant marker (or locus) in either one allele or both alleles of a gene (or a particular chromosome region). With respect to a particular nucleotide position of a gene of interest, the nucleotide(s) at that locus or equivalent thereof in one or both alleles form the genotype of the gene at that locus. A genotype can be homozygous or heterozygous. Accordingly, “genotyping” means determining the genotype, that is, the nucleotide(s) at a particular gene locus. Genotyping can also be done by determining the amino acid variant at a particular position of a protein which can be used to deduce the corresponding nucleotide variant(s).

The term “locus” refers to a specific position or site in a gene sequence or protein. Thus, there may be one or more contiguous nucleotides in a particular gene locus, or one or more amino acids at a particular locus in a polypeptide. Moreover, a locus may refer to a particular position in a gene where one or more nucleotides have been deleted, inserted, or inverted.

Unless specified otherwise or understood by one of skill in art, the terms “polypeptide,” “protein,” and “peptide” are used interchangeably herein to refer to an amino acid chain in which the amino acid residues are linked by covalent peptide bonds. The amino acid chain can be of any length of at least two amino acids, including full-length proteins. Unless otherwise specified, polypeptide, protein, and peptide also encompass various modified forms thereof, including but not limited to glycosylated forms, phosphorylated forms, etc. A polypeptide, protein or peptide can also be referred to as a gene product.

Lists of gene and gene products that can be assayed by molecular profiling techniques are presented herein. Lists of genes may be presented in the context of molecular profiling techniques that detect a gene product (e.g., an mRNA or protein). One of skill will understand that this implies detection of the gene product of the listed genes. Similarly, lists of gene products may be presented in the context of molecular profiling techniques that detect a gene sequence or copy number. One of skill will understand that this implies detection of the gene corresponding to the gene products, including as an example DNA encoding the gene products. As will be appreciated by those skilled in the art, a “biomarker” or “marker” comprises a gene and/or gene product depending on the context.

The terms “label” and “detectable label” can refer to any composition detectable by spectroscopic, photochemical, biochemical, immunochemical, electrical, optical, chemical or similar methods. Such labels include biotin for staining with labeled streptavidin conjugate, magnetic beads (e.g., DYNABEADS™), fluorescent dyes (e.g., fluorescein, Texas red, rhodamine, green fluorescent protein, and the like), radiolabels (e.g., ³H, ¹²⁵I, ³⁵S, ¹⁴C, or ³²P), enzymes (e.g., horse radish peroxidase, alkaline phosphatase and others commonly used in an ELISA), and calorimetric labels such as colloidal gold or colored glass or plastic (e.g., polystyrene, polypropylene, latex, etc) beads. Patents teaching the use of such labels include U.S. Pat. Nos. 3,817,837; 3,850,752; 3,939,350; 3,996,345; 4,277,437; 4,275,149; and 4,366,241. Means of detecting such labels are well known to those of skill in the art. Thus, for example, radiolabels may be detected using photographic film or scintillation counters, fluorescent markers may be detected using a photodetector to detect emitted light. Enzymatic labels are typically detected by providing the enzyme with a substrate and detecting the reaction product produced by the action of the enzyme on the substrate, and calorimetric labels are detected by simply visualizing the colored label. Labels can include, e.g., ligands that bind to labeled antibodies, fluorophores, chemiluminescent agents, enzymes, and antibodies which can serve as specific binding pair members for a labeled ligand. An introduction to labels, labeling procedures and detection of labels is found in Polak and Van Noorden Introduction to Immunocytochemistry, 2nd ed., Springer Verlag, N Y (1997); and in Haugland Handbook of Fluorescent Probes and Research Chemicals, a combined handbook and catalogue Published by Molecular Probes, Inc. (1996).

Detectable labels include, but are not limited to, nucleotides (labeled or unlabelled), compomers, sugars, peptides, proteins, antibodies, chemical compounds, conducting polymers, binding moieties such as biotin, mass tags, calorimetric agents, light emitting agents, chemiluminescent agents, light scattering agents, fluorescent tags, radioactive tags, charge tags (electrical or magnetic charge), volatile tags and hydrophobic tags, biomolecules (e.g., members of a binding pair antibody/antigen, antibody/antibody, antibody/antibody fragment, antibody/antibody receptor, antibody/protein A or protein G, hapten/anti-hapten, biotin/avidin, biotin/streptavidin, folic acid/folate binding protein, vitamin B12/intrinsic factor, chemical reactive group/complementary chemical reactive group (e.g., sulfhydryl/maleimide, sulfhydryl/haloacetyl derivative, amine/isotriocyanate, amine/succinimidyl ester, and amine/sulfonyl halides) and the like.

The term “antibody” as used herein encompasses naturally occurring antibodies as well as non-naturally occurring antibodies, including, for example, single chain antibodies, chimeric, bifunctional and humanized antibodies, as well as antigen-binding fragments thereof, (e.g., Fab′, F(ab′)₂, Fab, Fv and rIgG). See also, Pierce Catalog and Handbook, 1994-1995 (Pierce Chemical Co., Rockford, Ill.). See also, e.g., Kuby, J., Immunology, 3.sup.rd Ed., W. H. Freeman & Co., New York (1998). Such non-naturally occurring antibodies can be constructed using solid phase peptide synthesis, can be produced recombinantly or can be obtained, for example, by screening combinatorial libraries consisting of variable heavy chains and variable light chains as described by Huse et al., Science 246:1275-1281 (1989), which is incorporated herein by reference. These and other methods of making, for example, chimeric, humanized, CDR-grafted, single chain, and bifunctional antibodies are well known to those skilled in the art. See, e.g., Winter and Harris, Immunol. Today 14:243-246 (1993); Ward et al., Nature 341:544-546 (1989); Harlow and Lane, Antibodies, 511-52, Cold Spring Harbor Laboratory publications, New York, 1988; Hilyard et al., Protein Engineering: A practical approach (IRL Press 1992); Borrebaeck, Antibody Engineering, 2d ed. (Oxford University Press 1995); each of which is incorporated herein by reference.

Unless otherwise specified, antibodies can include both polyclonal and monoclonal antibodies. Antibodies also include genetically engineered forms such as chimeric antibodies (e.g., humanized murine antibodies) and heteroconjugate antibodies (e.g., bispecific antibodies). The term also refers to recombinant single chain Fv fragments (scFv). The term antibody also includes bivalent or bispecific molecules, diabodies, triabodies, and tetrabodies. Bivalent and bispecific molecules are described in, e.g., Kostelny et al. (1992) J Immunol 148:1547, Pack and Pluckthun (1992) Biochemistry 31:1579, Holliger et al. (1993) Proc Natl Acad Sci USA. 90:6444, Gruber et al. (1994) J Immuno1:5368, Zhu et al. (1997) Protein Sci 6:781, Hu et al. (1997) Cancer Res. 56:3055, Adams et al. (1993) Cancer Res. 53:4026, and McCartney, et al. (1995) Protein Eng. 8:301.

Typically, an antibody has a heavy and light chain. Each heavy and light chain contains a constant region and a variable region, (the regions are also known as “domains”). Light and heavy chain variable regions contain four framework regions interrupted by three hyper-variable regions, also called complementarity-determining regions (CDRs). The extent of the framework regions and CDRs have been defined. The sequences of the framework regions of different light or heavy chains are relatively conserved within a species. The framework region of an antibody, that is the combined framework regions of the constituent light and heavy chains, serves to position and align the CDRs in three dimensional spaces. The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located. Thus, a V_{H} CDR3 is located in the variable domain of the heavy chain of the antibody in which it is found, whereas a V_{L} CDR1 is the CDR1 from the variable domain of the light chain of the antibody in which it is found. References to V_{H} refer to the variable region of an immunoglobulin heavy chain of an antibody, including the heavy chain of an Fv, scFv, or Fab. References to V_{L} refer to the variable region of an immunoglobulin light chain, including the light chain of an Fv, scFv, dsFv or Fab.

The phrase “single chain Fv” or “scFv” refers to an antibody in which the variable domains of the heavy chain and of the light chain of a traditional two chain antibody have been joined to form one chain. Typically, a linker peptide is inserted between the two chains to allow for proper folding and creation of an active binding site. A “chimeric antibody” is an immunoglobulin molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity.

A “humanized antibody” is an immunoglobulin molecule that contains minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, a humanized antibody will comprise substantially all of at least one, and typically two, variable domains, in which all or substantially all of the CDR regions correspond to those of a non-human immunoglobulin and all or substantially all of the framework (FR) regions are those of a human immunoglobulin consensus sequence. The humanized antibody optimally also will comprise at least a portion of an immunoglobulin constant region (Fc), typically that of a human immunoglobulin (Jones et al., Nature 321:522-525 (1986); Riechmann et al., Nature 332:323-327 (1988); and Presta, Curr. Op. Struct. Biol. 2:593-596 (1992)) Humanization can be essentially performed following the method of Winter and co-workers (Jones et al., Nature 321:522-525 (1986); Riechmann et al., Nature 332:323-327 (1988); Verhoeyen et al., Science 239:1534-1536 (1988)), by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species.

The terms “epitope” and “antigenic determinant” refer to a site on an antigen to which an antibody binds. Epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5 or 8-10 amino acids in a unique spatial conformation. Methods of determining spatial conformation of epitopes include, for example, x-ray crystallography and 2-dimensional nuclear magnetic resonance. See, e.g., Epitope Mapping Protocols in Methods in Molecular Biology, Vol. 66, Glenn E. Morris, Ed (1996).

The terms “primer”, “probe,” and “oligonucleotide” are used herein interchangeably to refer to a relatively short nucleic acid fragment or sequence. They can comprise DNA, RNA, or a hybrid thereof, or chemically modified analog or derivatives thereof. Typically, they are single-stranded. However, they can also be double-stranded having two complementing strands which can be separated by denaturation. Normally, primers, probes and oligonucleotides have a length of from about 8 nucleotides to about 200 nucleotides, preferably from about 12 nucleotides to about 100 nucleotides, and more preferably about 18 to about 50 nucleotides. They can be labeled with detectable markers or modified using conventional manners for various molecular biological applications.

The term “isolated” when used in reference to nucleic acids (e.g., genomic DNAs, cDNAs, mRNAs, or fragments thereof) is intended to mean that a nucleic acid molecule is present in a form that is substantially separated from other naturally occurring nucleic acids that are normally associated with the molecule. Because a naturally existing chromosome (or a viral equivalent thereof) includes a long nucleic acid sequence, an isolated nucleic acid can be a nucleic acid molecule having only a portion of the nucleic acid sequence in the chromosome but not one or more other portions present on the same chromosome. More specifically, an isolated nucleic acid can include naturally occurring nucleic acid sequences that flank the nucleic acid in the naturally existing chromosome (or a viral equivalent thereof). An isolated nucleic acid can be substantially separated from other naturally occurring nucleic acids that are on a different chromosome of the same organism. An isolated nucleic acid can also be a composition in which the specified nucleic acid molecule is significantly enriched so as to constitute at least 10%, 20%, 30%, 40%, 50%, 60%, 70%, 80%, 90%, 95%, or at least 99% of the total nucleic acids in the composition.

An isolated nucleic acid can be a hybrid nucleic acid having the specified nucleic acid molecule covalently linked to one or more nucleic acid molecules that are not the nucleic acids naturally flanking the specified nucleic acid. For example, an isolated nucleic acid can be in a vector. In addition, the specified nucleic acid may have a nucleotide sequence that is identical to a naturally occurring nucleic acid or a modified form or mutein thereof having one or more mutations such as nucleotide substitution, deletion/insertion, inversion, and the like.

An isolated nucleic acid can be prepared from a recombinant host cell (in which the nucleic acids have been recombinantly amplified and/or expressed), or can be a chemically synthesized nucleic acid having a naturally occurring nucleotide sequence or an artificially modified form thereof.

The term “isolated polypeptide” as used herein is defined as a polypeptide molecule that is present in a form other than that found in nature. Thus, an isolated polypeptide can be a non-naturally occurring polypeptide. For example, an isolated polypeptide can be a “hybrid polypeptide.” An isolated polypeptide can also be a polypeptide derived from a naturally occurring polypeptide by additions or deletions or substitutions of amino acids. An isolated polypeptide can also be a “purified polypeptide” which is used herein to mean a composition or preparation in which the specified polypeptide molecule is significantly enriched so as to constitute at least 10% of the total protein content in the composition. A “purified polypeptide” can be obtained from natural or recombinant host cells by standard purification techniques, or by chemically synthesis, as will be apparent to skilled artisans.

The terms “hybrid protein,” “hybrid polypeptide,” “hybrid peptide,” “fusion protein,” “fusion polypeptide,” and “fusion peptide” are used herein interchangeably to mean a non-naturally occurring polypeptide or isolated polypeptide having a specified polypeptide molecule covalently linked to one or more other polypeptide molecules that do not link to the specified polypeptide in nature. Thus, a “hybrid protein” may be two naturally occurring proteins or fragments thereof linked together by a covalent linkage. A “hybrid protein” may also be a protein formed by covalently linking two artificial polypeptides together. Typically but not necessarily, the two or more polypeptide molecules are linked or “fused” together by a peptide bond forming a single non-branched polypeptide chain.

The term “high stringency hybridization conditions,” when used in connection with nucleic acid hybridization, includes hybridization conducted overnight at 42° C. in a solution containing 50% formamide, 5×SSC (750 mM NaCl, 75 mM sodium citrate), 50 mM sodium phosphate, pH 7.6, 5×Denhardt's solution, 10% dextran sulfate, and 20 microgram/ml denatured and sheared salmon sperm DNA, with hybridization filters washed in 0.1×SSC at about 65° C. The term “moderate stringent hybridization conditions,” when used in connection with nucleic acid hybridization, includes hybridization conducted overnight at 37° C. in a solution containing 50% formamide, 5×SSC (750 mM NaCl, 75 mM sodium citrate), 50 mM sodium phosphate, pH 7.6, 5×Denhardt's solution, 10% dextran sulfate, and 20 microgram/ml denatured and sheared salmon sperm DNA, with hybridization filters washed in 1×SSC at about 50° C. It is noted that many other hybridization methods, solutions and temperatures can be used to achieve comparable stringent hybridization conditions as will be apparent to skilled artisans.

For the purpose of comparing two different nucleic acid or polypeptide sequences, one sequence (test sequence) may be described to be a specific percentage identical to another sequence (comparison sequence). The percentage identity can be determined by the algorithm of Karlin and Altschul, Proc. Natl. Acad. Sci. USA, 90:5873-5877 (1993), which is incorporated into various BLAST programs. The percentage identity can be determined by the “BLAST 2 Sequences” tool, which is available at the National Center for Biotechnology Information (NCBI) website. See Tatusova and Madden, FEMS Microbiol. Lett., 174(2):247-250 (1999). For pairwise DNA-DNA comparison, the BLASTN program is used with default parameters (e.g., Match: 1; Mismatch: −2; Open gap: 5 penalties; extension gap: 2 penalties; gap x_dropoff: 50; expect: 10; and word size: 11, with filter). For pairwise protein-protein sequence comparison, the BLASTP program can be employed using default parameters (e.g., Matrix: BLOSUM62; gap open: 11; gap extension: 1; x_dropoff: 15; expect: 10.0; and wordsize: 3, with filter). Percent identity of two sequences is calculated by aligning a test sequence with a comparison sequence using BLAST, determining the number of amino acids or nucleotides in the aligned test sequence that are identical to amino acids or nucleotides in the same position of the comparison sequence, and dividing the number of identical amino acids or nucleotides by the number of amino acids or nucleotides in the comparison sequence. When BLAST is used to compare two sequences, it aligns the sequences and yields the percent identity over defined, aligned regions. If the two sequences are aligned across their entire length, the percent identity yielded by the BLAST is the percent identity of the two sequences. If BLAST does not align the two sequences over their entire length, then the number of identical amino acids or nucleotides in the unaligned regions of the test sequence and comparison sequence is considered to be zero and the percent identity is calculated by adding the number of identical amino acids or nucleotides in the aligned regions and dividing that number by the length of the comparison sequence. Various versions of the BLAST programs can be used to compare sequences, e.g., BLAST 2.1.2 or BLAST+2.2.22.

A subject or individual can be any animal which may benefit from the methods of the invention, including, e.g., humans and non-human mammals, such as primates, rodents, horses, dogs and cats. Subjects include without limitation a eukaryotic organisms, most preferably a mammal such as a primate, e.g., chimpanzee or human, cow; dog; cat; a rodent, e.g., guinea pig, rat, mouse; rabbit; or a bird; reptile; or fish. Subjects specifically intended for treatment using the methods described herein include humans. A subject may be referred to as an individual or a patient.

Treatment of a disease or individual according to the invention is an approach for obtaining beneficial or desired medical results, including clinical results, but not necessarily a cure. For purposes of this invention, beneficial or desired clinical results include, but are not limited to, alleviation or amelioration of one or more symptoms, diminishment of extent of disease, stabilized (i.e., not worsening) state of disease, preventing spread of disease, delay or slowing of disease progression, amelioration or palliation of the disease state, and remission (whether partial or total), whether detectable or undetectable. Treatment also includes prolonging survival as compared to expected survival if not receiving treatment or if receiving a different treatment. A treatment can include administration of a therapeutic agent, which can be an agent that exerts a cytotoxic, cytostatic, or immunomodulatory effect on diseased cells, e.g., cancer cells, or other cells that may promote a diseased state, e.g., activated immune cells. Therapeutic agents selected by the methods of the invention are not limited. Any therapeutic agent can be selected where a link can be made between molecular profiling and potential efficacy of the agent. Therapeutic agents include without limitation drugs, pharmaceuticals, small molecules, protein therapies, antibody therapies, viral therapies, gene therapies, and the like. Cancer treatments or therapies include apoptosis-mediated and non-apoptosis mediated cancer therapies including, without limitation, chemotherapy, hormonal therapy, radiotherapy, immunotherapy, and combinations thereof. Chemotherapeutic agents comprise therapeutic agents and combinations of therapeutic agents that treat, cancer cells, e.g., by killing those cells. Examples of different types of chemotherapeutic drugs include without limitation alkylating agents (e.g., nitrogen mustard derivatives, ethylenimines, alkylsulfonates, hydrazines and triazines, nitrosureas, and metal salts), plant alkaloids (e.g., vinca alkaloids, taxanes, podophyllotoxins, and camptothecan analogs), antitumor antibiotics (e.g., anthracyclines, chromomycins, and the like), antimetabolites (e.g., folic acid antagonists, pyrimidine antagonists, purine antagonists, and adenosine deaminase inhibitors), topoisomerase I inhibitors, topoisomerase II inhibitors, and miscellaneous antineoplastics (e.g., ribonucleotide reductase inhibitors, adrenocortical steroid inhibitors, enzymes, antimicrotubule agents, and retinoids).

A biomarker refers generally to a molecule, including without limitation a gene or product thereof, nucleic acids (e.g., DNA, RNA), protein/peptide/polypeptide, carbohydrate structure, lipid, glycolipid, characteristics of which can be detected in a tissue or cell to provide information that is predictive, diagnostic, prognostic and/or theranostic for sensitivity or resistance to candidate treatment.

Biological Samples

A sample as used herein includes any relevant biological sample that can be used for molecular profiling, e.g., sections of tissues such as biopsy or tissue removed during surgical or other procedures, bodily fluids, autopsy samples, and frozen sections taken for histological purposes. Such samples include blood and blood fractions or products (e.g., serum, buffy coat, plasma, platelets, red blood cells, and the like), sputum, malignant effusion, cheek cells tissue, cultured cells (e.g., primary cultures, explants, and transformed cells), stool, urine, other biological or bodily fluids (e.g., prostatic fluid, gastric fluid, intestinal fluid, renal fluid, lung fluid, cerebrospinal fluid, and the like), etc. The sample can comprise biological material that is a fresh frozen & formalin fixed paraffin embedded (FFPE) block, formalin-fixed paraffin embedded, or is within an RNA preservative+formalin fixative. More than one sample of more than one type can be used for each patient. In a preferred embodiment, the sample comprises a fixed tumor sample.

The sample used in the methods described herein can be a formalin fixed paraffin embedded (FFPE) sample. The FFPE sample can be one or more of fixed tissue, unstained slides, bone marrow core or clot, core needle biopsy, malignant fluids and fine needle aspirate (FNA). In an embodiment, the fixed tissue comprises a tumor containing formalin fixed paraffin embedded (FFPE) block from a surgery or biopsy. In another embodiment, the unstained slides comprise unstained, charged, unbaked slides from a paraffin block. In another embodiment, bone marrow core or clot comprises a decalcified core. A formalin fixed core and/or clot can be paraffin-embedded. In still another embodiment, the core needle biopsy comprises 1, 2, 3, 4, 5, 6, 7, 8, 9, 10 or more, e.g., 3-4, paraffin embedded biopsy samples. An 18 gauge needle biopsy can be used. The malignant fluid can comprise a sufficient volume of fresh pleural/ascitic fluid to produce a 5×5×2 mm cell pellet. The fluid can be formalin fixed in a paraffin block. In an embodiment, the core needle biopsy comprises 1, 2, 3, 4, 5, 6, 7, 8, 9, 10 or more, e.g., 4-6, paraffin embedded aspirates.

A sample may be processed according to techniques understood by those in the art. A sample can be without limitation fresh, frozen or fixed cells or tissue. In some embodiments, a sample comprises formalin-fixed paraffm-embedded (FFPE) tissue, fresh tissue or fresh frozen (FF) tissue. A sample can comprise cultured cells, including primary or immortalized cell lines derived from a subject sample. A sample can also refer to an extract from a sample from a subject. For example, a sample can comprise DNA, RNA or protein extracted from a tissue or a bodily fluid. Many techniques and commercial kits are available for such purposes. The fresh sample from the individual can be treated with an agent to preserve RNA prior to further processing, e.g., cell lysis and extraction. Samples can include frozen samples collected for other purposes. Samples can be associated with relevant information such as age, gender, and clinical symptoms present in the subject; source of the sample; and methods of collection and storage of the sample. A sample is typically obtained from a subject.

A biopsy comprises the process of removing a tissue sample for diagnostic or prognostic evaluation, and to the tissue specimen itself. Any biopsy technique known in the art can be applied to the molecular profiling methods of the present invention. The biopsy technique applied can depend on the tissue type to be evaluated (e.g., colon, prostate, kidney, bladder, lymph node, liver, bone marrow, blood cell, lung, breast, etc.), the size and type of the tumor (e.g., solid or suspended, blood or ascites), among other factors. Representative biopsy techniques include, but are not limited to, excisional biopsy, incisional biopsy, needle biopsy, surgical biopsy, and bone marrow biopsy. An “excisional biopsy” refers to the removal of an entire tumor mass with a small margin of normal tissue surrounding it. An “incisional biopsy” refers to the removal of a wedge of tissue that includes a cross-sectional diameter of the tumor. Molecular profiling can use a “core-needle biopsy” of the tumor mass, or a “fine-needle aspiration biopsy” which generally obtains a suspension of cells from within the tumor mass. Biopsy techniques are discussed, for example, in Harrison's Principles of Internal Medicine, Kasper, et al., eds., 16th ed., 2005, Chapter 70, and throughout Part V.

Standard molecular biology techniques known in the art and not specifically described are generally followed as in Sambrook et al., Molecular Cloning: A Laboratory Manual, Cold Spring Harbor Laboratory Press, New York (1989), and as in Ausubel et al., Current Protocols in Molecular Biology, John Wiley and Sons, Baltimore, Md. (1989) and as in Perbal, A Practical Guide to Molecular Cloning, John Wiley & Sons, New York (1988), and as in Watson et al., Recombinant DNA, Scientific American Books, New York and in Birren et al (eds) Genome Analysis: A Laboratory Manual Series, Vols. 1-4 Cold Spring Harbor Laboratory Press, New York (1998) and methodology as set forth in U.S. Pat. Nos. 4,666,828; 4,683,202; 4,801,531; 5,192,659 and 5,272,057 and incorporated herein by reference. Polymerase chain reaction (PCR) can be carried out generally as in PCR Protocols: A Guide to Methods and Applications, Academic Press, San Diego, Calif. (1990).

### Vesicles

The sample can comprise vesicles. Methods of the invention can include assessing one or more vesicles, including assessing vesicle populations. A vesicle, as used herein, is a membrane vesicle that is shed from cells. Vesicles or membrane vesicles include without limitation: circulating microvesicles (cMVs), microvesicle, exosome, nanovesicle, dexosome, bleb, blebby, prostasome, microparticle, intralumenal vesicle, membrane fragment, intralumenal endosomal vesicle, endosomal-like vesicle, exocytosis vehicle, endosome vesicle, endosomal vesicle, apoptotic body, multivesicular body, secretory vesicle, phospholipid vesicle, liposomal vesicle, argosome, texasome, secresome, tolerosome, melanosome, oncosome, or exocytosed vehicle. Furthermore, although vesicles may be produced by different cellular processes, the methods of the invention are not limited to or reliant on any one mechanism, insofar as such vesicles are present in a biological sample and are capable of being characterized by the methods disclosed herein. Unless otherwise specified, methods that make use of a species of vesicle can be applied to other types of vesicles. Vesicles comprise spherical structures with a lipid bilayer similar to cell membranes which surrounds an inner compartment which can contain soluble components, sometimes referred to as the payload. In some embodiments, the methods of the invention make use of exosomes, which are small secreted vesicles of about 40-100 nm in diameter. For a review of membrane vesicles, including types and characterizations, see Thery et al., Nat Rev Immunol. 2009 August; 9(8):581-93. Some properties of different types of vesicles include those in Table 1:

TABLE 1
Vesicle Properties

| | | | | | Exosome- | |
|---|---|---|---|---|---|---|
| | | | | Membrane | like | Apoptotic |
| Feature | Exosomes | Microvesicles | Ectosomes | particles | vesicles | vesicles |
| Size | 50-100 nm | 100-1,000 nm | 50-200 nm | 50-80 nm | 20-50 nm | 50-500 nm |
| Density in | 1.13-1.19 g/ml | | | 1.04-1.07 | 1.1 g/ml | 1.16-1.28 |
| sucrose | | | | g/ml | | g/ml |
| EM | Cup shape | Irregular | Bilamellar | Round | Irregular | Heterogeneous |
| appearance | | shape, | round | | shape | |
| | | electron | structures | | | |
| | | dense | | | | |
| Sedimentation | 100,000 g | 10,000 g | 160,000-200,000 g | 100,000-200,000 g | 175,000 g | 1,200 g, |
| | | | | | | 10,000 g, |
| | | | | | | 100,000 g |
| Lipid | Enriched in | Expose PPS | Enriched in | | No lipid | |
| composition | cholesterol, | | cholesterol | | rafts | |
| | sphingomyelin | | and | | | |
| | and ceramide; | | diacylglycero1; | | | |
| | contains lipid | | expose PPS | | | |
| | rafts; expose | | | | | |
| | PPS | | | | | |
| Major | Tetraspanins | Integrins, | CR1 and | CD133; no | TNFRI | Histones |
| protein | (e.g., CD63, | selectins and | proteolytic | CD63 | | |
| markers | CD9), Alix, | CD40 ligand | enzymes; no | | | |
| | TSG101 | | CD63 | | | |
| Intracellular | Internal | Plasma | Plasma | Plasma | | |
| origin | compartments | membrane | membrane | membrane | | |
| | (endosomes) | | | | | |

Abbreviations:
phosphatidylserine (PPS);
electron microscopy (EM)

Vesicles include shed membrane bound particles, or “microparticles,” that are derived from either the plasma membrane or an internal membrane. Vesicles can be released into the extracellular environment from cells. Cells releasing vesicles include without limitation cells that originate from, or are derived from, the ectoderm, endoderm, or mesoderm. The cells may have undergone genetic, environmental, and/or any other variations or alterations. For example, the cell can be tumor cells. A vesicle can reflect any changes in the source cell, and thereby reflect changes in the originating cells, e.g., cells having various genetic mutations. In one mechanism, a vesicle is generated intracellularly when a segment of the cell membrane spontaneously invaginates and is ultimately exocytosed (see for example, Keller et al., Immunol. Lett. 107 (2): 102-8 (2006)). Vesicles also include cell-derived structures bounded by a lipid bilayer membrane arising from both herniated evagination (blebbing) separation and sealing of portions of the plasma membrane or from the export of any intracellular membrane-bounded vesicular structure containing various membrane-associated proteins of tumor origin, including surface-bound molecules derived from the host circulation that bind selectively to the tumor-derived proteins together with molecules contained in the vesicle lumen, including but not limited to tumor-derived microRNAs or intracellular proteins. Blebs and blebbing are further described in Charras et al., Nature Reviews Molecular and Cell Biology, Vol. 9, No. 11, p. 730-736 (2008). A vesicle shed into circulation or bodily fluids from tumor cells may be referred to as a “circulating tumor-derived vesicle.” When such vesicle is an exosome, it may be referred to as a circulating-tumor derived exosome (CTE). In some instances, a vesicle can be derived from a specific cell of origin. CTE, as with a cell-of-origin specific vesicle, typically have one or more unique biomarkers that permit isolation of the CTE or cell-of-origin specific vesicle, e.g., from a bodily fluid and sometimes in a specific manner. For example, a cell or tissue specific markers are used to identify the cell of origin. Examples of such cell or tissue specific markers are disclosed herein and can further be accessed in the Tissue-specific Gene Expression and Regulation (TiGER) Database, available at bioinfo.wilmer.jhu.edu/tigee; Liu et al. (2008) TiGER: a database for tissue-specific gene expression and regulation. BMC Bioinformatics. 9:271; TissueDistributionDBs, available at genome.dkfz-heidelberg.de/menu/tissue_db/index.html.

A vesicle can have a diameter of greater than about 10 nm, 20 nm, or 30 nm. A vesicle can have a diameter of greater than 40 nm, 50 nm, 100 nm, 200 nm, 500 nm, 1000 nm or greater than 10,000 nm. A vesicle can have a diameter of about 30-1000 nm, about 30-800 nm, about 30-200 nm, or about 30-100 nm. In some embodiments, the vesicle has a diameter of less than 10,000 nm, 1000 nm, 800 nm, 500 nm, 200 nm, 100 nm, 50 nm, 40 nm, 30 nm, 20 nm or less than 10 nm. As used herein the term “about” in reference to a numerical value means that variations of 10% above or below the numerical value are within the range ascribed to the specified value. Typical sizes for various types of vesicles are shown in Table 1. Vesicles can be assessed to measure the diameter of a single vesicle or any number of vesicles. For example, the range of diameters of a vesicle population or an average diameter of a vesicle population can be determined. Vesicle diameter can be assessed using methods known in the art, e.g., imaging technologies such as electron microscopy. In an embodiment, a diameter of one or more vesicles is determined using optical particle detection. See, e.g., U.S. Pat. No. 7,751,053, entitled “Optical Detection and Analysis of Particles” and issued Jul. 6, 2010; and U.S. Pat. No. 7,399,600, entitled “Optical Detection and Analysis of Particles” and issued Jul. 15, 2010.

In some embodiments, vesicles are directly assayed from a biological sample without prior isolation, purification, or concentration from the biological sample. For example, the amount of vesicles in the sample can by itself provide a biosignature that provides a diagnostic, prognostic or theranostic determination. Alternatively, the vesicle in the sample may be isolated, captured, purified, or concentrated from a sample prior to analysis. As noted, isolation, capture or purification as used herein comprises partial isolation, partial capture or partial purification apart from other components in the sample. Vesicle isolation can be performed using various techniques as described herein or known in the art, including without limitation size exclusion chromatography, density gradient centrifugation, differential centrifugation, nanomembrane ultrafiltration, immunoabsorbent capture, affinity purification, affinity capture, immunoassay, immunoprecipitation, microfluidic separation, flow cytometry or combinations thereof.

Vesicles can be assessed to provide a phenotypic characterization by comparing vesicle characteristics to a reference. In some embodiments, surface antigens on a vesicle are assessed. A vesicle or vesicle population carrying a specific marker can be referred to as a positive (biomarker+) vesicle or vesicle population. For example, a DLL4+population refers to a vesicle population associated with DLL4. Conversely, a DLL4− population would not be associated with DLL4. The surface antigens can provide an indication of the anatomical origin and/or cellular of the vesicles and other phenotypic information, e.g., tumor status. For example, vesicles found in a patient sample can be assessed for surface antigens indicative of colorectal origin and the presence of cancer, thereby identifying vesicles associated with colorectal cancer cells. The surface antigens may comprise any informative biological entity that can be detected on the vesicle membrane surface, including without limitation surface proteins, lipids, carbohydrates, and other membrane components. For example, positive detection of colon derived vesicles expressing tumor antigens can indicate that the patient has colorectal cancer. As such, methods of the invention can be used to characterize any disease or condition associated with an anatomical or cellular origin, by assessing, for example, disease-specific and cell-specific biomarkers of one or more vesicles obtained from a subject.

In embodiments, one or more vesicle payloads are assessed to provide a phenotypic characterization. The payload with a vesicle comprises any informative biological entity that can be detected as encapsulated within the vesicle, including without limitation proteins and nucleic acids, e.g., genomic or cDNA, mRNA, or functional fragments thereof, as well as microRNAs (miRs). In addition, methods of the invention are directed to detecting vesicle surface antigens (in addition or exclusive to vesicle payload) to provide a phenotypic characterization. For example, vesicles can be characterized by using binding agents (e.g., antibodies or aptamers) that are specific to vesicle surface antigens, and the bound vesicles can be further assessed to identify one or more payload components disclosed therein. As described herein, the levels of vesicles with surface antigens of interest or with payload of interest can be compared to a reference to characterize a phenotype. For example, overexpression in a sample of cancer-related surface antigens or vesicle payload, e.g., a tumor associated mRNA or microRNA, as compared to a reference, can indicate the presence of cancer in the sample. The biomarkers assessed can be present or absent, increased or reduced based on the selection of the desired target sample and comparison of the target sample to the desired reference sample. Non-limiting examples of target samples include: disease; treated/not-treated; different time points, such as a in a longitudinal study; and non-limiting examples of reference sample: non-disease; normal; different time points; and sensitive or resistant to candidate treatment(s).

In an embodiment, molecular profiling of the invention comprises analysis of microvesicles, such as circulating microvesicles.

### MicroRNA

Various biomarker molecules can be assessed in biological samples or vesicles obtained from such biological samples. MicroRNAs comprise one class biomarkers assessed via methods of the invention. MicroRNAs, also referred to herein as miRNAs or miRs, are short RNA strands approximately 21-23 nucleotides in length. MiRNAs are encoded by genes that are transcribed from DNA but are not translated into protein and thus comprise non-coding RNA. The miRs are processed from primary transcripts known as pri-miRNA to short stem-loop structures called pre-miRNA and fmally to the resulting single strand miRNA. The pre-miRNA typically forms a structure that folds back on itself in self-complementary regions. These structures are then processed by the nuclease Dicer in animals or DCL1 in plants. Mature miRNA molecules are partially complementary to one or more messenger RNA (mRNA) molecules and can function to regulate translation of proteins. Identified sequences of miRNA can be accessed at publicly available databases, such as www.microRNA.org, www.mirbase.org, or www.mirz.unibas.ch/cgi/miRNA.cgi.

miRNAs are generally assigned a number according to the naming convention “mir-[number].” The number of a miRNA is assigned according to its order of discovery relative to previously identified miRNA species. For example, if the last published miRNA was mir-121, the next discovered miRNA will be named mir-122, etc. When a miRNA is discovered that is homologous to a known miRNA from a different organism, the name can be given an optional organism identifier, of the form [organism identifier]-mir-[number]. Identifiers include hsa for Homo sapiens and mmu for Mus Musculus. For example, a human homolog to mir-121 might be referred to as hsa-mir-121 whereas the mouse homolog can be referred to as mmu-mir-121.

Mature microRNA is commonly designated with the prefix “miR” whereas the gene or precursor miRNA is designated with the prefix “mir.” For example, mir-121 is a precursor for miR-121. When differing miRNA genes or precursors are processed into identical mature miRNAs, the genes/precursors can be delineated by a numbered suffix. For example, mir-121-1 and mir-121-2 can refer to distinct genes or precursors that are processed into miR-121. Lettered suffixes are used to indicate closely related mature sequences. For example, mir-121a and mir-121b can be processed to closely related miRNAs miR-121a and miR-121b, respectively. In the context of the invention, any microRNA (miRNA or miR) designated herein with the prefix mir-* or miR-* is understood to encompass both the precursor and/or mature species, unless otherwise explicitly stated otherwise.

Sometimes it is observed that two mature miRNA sequences originate from the same precursor. When one of the sequences is more abundant that the other, a “*” suffix can be used to designate the less common variant. For example, miR-121 would be the predominant product whereas miR-121* is the less common variant found on the opposite arm of the precursor. If the predominant variant is not identified, the miRs can be distinguished by the suffix “5p” for the variant from the 5′ arm of the precursor and the suffix “3p” for the variant from the 3′ arm. For example, miR-121-5p originates from the 5′ arm of the precursor whereas miR-121-3p originates from the 3′ arm. Less commonly, the 5p and 3p variants are referred to as the sense (“s”) and anti-sense (“as”) forms, respectively. For example, miR-121-5p may be referred to as miR-121-s whereas miR-121-3p may be referred to as miR-121-as.

The above naming conventions have evolved over time and are general guidelines rather than absolute rules. For example, the let- and lin-families of miRNAs continue to be referred to by these monikers. The mir/miR convention for precursor/mature forms is also a guideline and context should be taken into account to determine which form is referred to. Further details of miR naming can be found at www.mirbase.org or Ambros et al., A uniform system for microRNA annotation, RNA 9:277-279 (2003).

Plant miRNAs follow a different naming convention as described in Meyers et al., Plant Cell. 2008 20(12):3186-3190.

A number of miRNAs are involved in gene regulation, and miRNAs are part of a growing class of non-coding RNAs that is now recognized as a major tier of gene control. In some cases, miRNAs can interrupt translation by binding to regulatory sites embedded in the 3′-UTRs of their target mRNAs, leading to the repression of translation. Target recognition involves complementary base pairing of the target site with the miRNA's seed region (positions 2-8 at the miRNA's 5′ end), although the exact extent of seed complementarity is not precisely determined and can be modified by 3′ pairing. In other cases, miRNAs function like small interfering RNAs (siRNA) and bind to perfectly complementary mRNA sequences to destroy the target transcript.

Characterization of a number of miRNAs indicates that they influence a variety of processes, including early development, cell proliferation and cell death, apoptosis and fat metabolism. For example, some miRNAs, such as lin-4, let-7, mir-14, mir-23, and bantam, have been shown to play critical roles in cell differentiation and tissue development. Others are believed to have similarly important roles because of their differential spatial and temporal expression patterns.

The miRNA database available at miRBase (www.mirbase.org) comprises a searchable database of published miRNA sequences and annotation. Further information about miRBase can be found in the following articles, each of which is incorporated by reference in its entirety herein: Griffiths-Jones et al., miRBase: tools for microRNA genomics. NAR 2008 36(Database Issue):D154-D158; Griffiths-Jones et al., miRBase: microRNA sequences, targets and gene nomenclature. NAR 2006 34 (Database Issue):D140-D144; and Griffiths-Jones, S. The microRNA Registry. NAR 2004 32 (Database Issue):D109-D111. Representative miRNAs contained in Release 16 of miRBase, made available September 2010.

As described herein, microRNAs are known to be involved in cancer and other diseases and can be assessed in order to characterize a phenotype in a sample. See, e.g., Ferracin et al., Micromarkers: miRNAs in cancer diagnosis and prognosis, Exp Rev Mol Diag, April 2010, Vol. 10, No. 3, Pages 297-308; Fabbri, miRNAs as molecular biomarkers of cancer, Exp Rev Mol Diag, May 2010, Vol. 10, No. 4, Pages 435-444.

In an embodiment, molecular profiling of the invention comprises analysis of microRNA.

Techniques to isolate and characterize vesicles and miRs are known to those of skill in the art. In addition to the methodology presented herein, additional methods can be found in U.S. Pat. No. 7,888,035, entitled “METHODS FOR ASSESSING RNA PATTERNS” and issued Feb. 15, 2011; and U.S. Pat. No. 7,897,356, entitled “METHODS AND SYSTEMS OF USING EXOSOMES FOR DETERMINING PHENOTYPES” and issued Mar. 1, 2011; and International Patent Publication Nos. WO/2011/066589, entitled “METHODS AND SYSTEMS FOR ISOLATING, STORING, AND ANALYZING VESICLES” and filed Nov. 30, 2010; WO/2011/088226, entitled “DETECTION OF GASTROINTESTINAL DISORDERS” and filed Jan. 13, 2011; WO/2011/109440, entitled “BIOMARKERS FOR THERANOSTICS” and filed Mar. 1, 2011; and WO/2011/127219, entitled “CIRCULATING BIOMARKERS FOR DISEASE” and filed Apr. 6, 2011, each of which applications are incorporated by reference herein in their entirety.

### Circulating Biomarkers

Circulating biomarkers include biomarkers that are detectable in body fluids, such as blood, plasma, serum. Examples of circulating cancer biomarkers include cardiac troponin T (cTnT), prostate specific antigen (PSA) for prostate cancer and CA125 for ovarian cancer. Circulating biomarkers according to the invention include any appropriate biomarker that can be detected in bodily fluid, including without limitation protein, nucleic acids, e.g., DNA, mRNA and microRNA, lipids, carbohydrates and metabolites. Circulating biomarkers can include biomarkers that are not associated with cells, such as biomarkers that are membrane associated, embedded in membrane fragments, part of a biological complex, or free in solution. In one embodiment, circulating biomarkers are biomarkers that are associated with one or more vesicles present in the biological fluid of a subject.

Circulating biomarkers have been identified for use in characterization of various phenotypes, such as detection of a cancer. See, e.g., Ahmed N, et al., Proteomic-based identification of haptoglobin-1 precursor as a novel circulating biomarker of ovarian cancer. Br. J. Cancer 2004; Mathelin et al., Circulating proteinic biomarkers and breast cancer, Gynecol Obstet Fertil. 2006 July-August; 34(7-8):638-46. Epub 2006 Jul. 28; Ye et al., Recent technical strategies to identify diagnostic biomarkers for ovarian cancer. Expert Rev Proteomics. 2007 February; 4(1):121-31; Carney, Circulating oncoproteins HER2/neu, EGFR and CAIX (MN) as novel cancer biomarkers. Expert Rev Mol Diagn. 2007 May; 7(3):309-19; Gagnon, Discovery and application of protein biomarkers for ovarian cancer, Curr Opin Obstet Gynecol. 2008 February; 20(1):9-13; Pasterkamp et al., Immune regulatory cells: circulating biomarker factories in cardiovascular disease. Clin Sci (Loud). 2008 August; 115(4):129-31; Fabbri, miRNAs as molecular biomarkers of cancer, Exp Rev Mol Diag, May 2010, Vol. 10, No. 4, Pages 435-444; PCT Patent Publication WO/2007/088537; U.S. Pat. Nos. 7,745,150 and 7,655,479; U.S. Patent Publications 20110008808, 20100330683, 20100248290, 20100222230, 20100203566, 20100173788, 20090291932, 20090239246, 20090226937, 20090111121, 20090004687, 20080261258, 20080213907, 20060003465, 20050124071, and 20040096915, each of which publication is incorporated herein by reference in its entirety. In an embodiment, molecular profiling of the invention comprises analysis of circulating biomarkers.

### Gene Expression Profiling

The methods and systems of the invention comprise expression profiling, which includes assessing differential expression of one or more target genes disclosed herein. Differential expression can include overexpression and/or underexpression of a biological product, e.g., a gene, mRNA or protein, compared to a control (or a reference). The control can include similar cells to the sample but without the disease (e.g., expression profiles obtained from samples from healthy individuals). A control can be a previously determined level that is indicative of a drug target efficacy associated with the particular disease and the particular drug target. The control can be derived from the same patient, e.g., a normal adjacent portion of the same organ as the diseased cells, the control can be derived from healthy tissues from other patients, or previously determined thresholds that are indicative of a disease responding or not-responding to a particular drug target. The control can also be a control found in the same sample, e.g. a housekeeping gene or a product thereof (e.g., mRNA or protein). For example, a control nucleic acid can be one which is known not to differ depending on the cancerous or non-cancerous state of the cell. The expression level of a control nucleic acid can be used to normalize signal levels in the test and reference populations. Illustrative control genes include, but are not limited to, e.g., β-actin, glyceraldehyde 3-phosphate dehydrogenase and ribosomal protein P1. Multiple controls or types of controls can be used. The source of differential expression can vary. For example, a gene copy number may be increased in a cell, thereby resulting in increased expression of the gene. Alternately, transcription of the gene may be modified, e.g., by chromatin remodeling, differential methylation, differential expression or activity of transcription factors, etc. Translation may also be modified, e.g., by differential expression of factors that degrade mRNA, translate mRNA, or silence translation, e.g., microRNAs or siRNAs. In some embodiments, differential expression comprises differential activity. For example, a protein may carry a mutation that increases the activity of the protein, such as constitutive activation, thereby contributing to a diseased state. Molecular profiling that reveals changes in activity can be used to guide treatment selection.

Methods of gene expression profiling include methods based on hybridization analysis of polynucleotides, and methods based on sequencing of polynucleotides. Commonly used methods known in the art for the quantification of mRNA expression in a sample include northern blotting and in situ hybridization (Parker & Barnes (1999) Methods in Molecular Biology 106:247-283); RNAse protection assays (Hod (1992) Biotechniques 13:852-854); and reverse transcription polymerase chain reaction (RT-PCR) (Weis et al. (1992) Trends in Genetics 8:263-264). Alternatively, antibodies may be employed that can recognize specific duplexes, including DNA duplexes, RNA duplexes, and DNA-RNA hybrid duplexes or DNA-protein duplexes. Representative methods for sequencing-based gene expression analysis include Serial Analysis of Gene Expression (SAGE), gene expression analysis by massively parallel signature sequencing (MPSS) and/or next generation sequencing.

RT-PCR

Reverse transcription polymerase chain reaction (RT-PCR) is a variant of polymerase chain reaction (PCR). According to this technique, a RNA strand is reverse transcribed into its DNA complement (i.e., complementary DNA, or cDNA) using the enzyme reverse transcriptase, and the resulting cDNA is amplified using PCR. Real-time polymerase chain reaction is another PCR variant, which is also referred to as quantitative PCR, Q-PCR, qRT-PCR, or sometimes as RT-PCR. Either the reverse transcription PCR method or the real-time PCR method can be used for molecular profiling according to the invention, and RT-PCR can refer to either unless otherwise specified or as understood by one of skill in the art.

RT-PCR can be used to determine RNA levels, e.g., mRNA or miRNA levels, of the biomarkers of the invention. RT-PCR can be used to compare such RNA levels of the biomarkers of the invention in different sample populations, in normal and tumor tissues, with or without drug treatment, to characterize patterns of gene expression, to discriminate between closely related RNAs, and to analyze RNA structure.

The first step is the isolation of RNA, e.g., mRNA, from a sample. The starting material can be total RNA isolated from human tumors or tumor cell lines, and corresponding normal tissues or cell lines, respectively. Thus RNA can be isolated from a sample, e.g., tumor cells or tumor cell lines, and compared with pooled DNA from healthy donors. If the source of mRNA is a primary tumor, mRNA can be extracted, for example, from frozen or archived paraffin-embedded and fixed (e.g. formalin-fixed) tissue samples.

General methods for mRNA extraction are well known in the art and are disclosed in standard textbooks of molecular biology, including Ausubel et al. (1997) Current Protocols of Molecular Biology, John Wiley and Sons. Methods for RNA extraction from paraffin embedded tissues are disclosed, for example, in Rupp & Locker (1987) Lab Invest. 56:A67, and De Andres et al., BioTechniques 18:42044 (1995). In particular, RNA isolation can be performed using purification kit, buffer set and protease from commercial manufacturers, such as Qiagen, according to the manufacturer's instructions (QIAGEN Inc., Valencia, Calif.). For example, total RNA from cells in culture can be isolated using Qiagen RNeasy mini-columns Numerous RNA isolation kits are commercially available and can be used in the methods of the invention.

In the alternative, the first step is the isolation of miRNA from a target sample. The starting material is typically total RNA isolated from human tumors or tumor cell lines, and corresponding normal tissues or cell lines, respectively. Thus RNA can be isolated from a variety of primary tumors or tumor cell lines, with pooled DNA from healthy donors. If the source of miRNA is a primary tumor, miRNA can be extracted, for example, from frozen or archived paraffin-embedded and fixed (e.g. formalin-fixed) tissue samples.

General methods for miRNA extraction are well known in the art and are disclosed in standard textbooks of molecular biology, including Ausubel et al. (1997) Current Protocols of Molecular Biology, John Wiley and Sons. Methods for RNA extraction from paraffin embedded tissues are disclosed, for example, in Rupp & Locker (1987) Lab Invest. 56:A67, and De Andres et al., BioTechniques 18:42044 (1995). In particular, RNA isolation can be performed using purification kit, buffer set and protease from commercial manufacturers, such as Qiagen, according to the manufacturer's instructions. For example, total RNA from cells in culture can be isolated using Qiagen RNeasy mini-columns Numerous miRNA isolation kits are commercially available and can be used in the methods of the invention.

Whether the RNA comprises mRNA, miRNA or other types of RNA, gene expression profiling by RT-PCR can include reverse transcription of the RNA template into cDNA, followed by amplification in a PCR reaction. Commonly used reverse transcriptases include, but are not limited to, avilo myeloblastosis virus reverse transcriptase (AMV-RT) and Moloney murine leukemia virus reverse transcriptase (MMLV-RT). The reverse transcription step is typically primed using specific primers, random hexamers, or oligo-dT primers, depending on the circumstances and the goal of expression profiling. For example, extracted RNA can be reverse-transcribed using a GeneAmp RNA PCR kit (Perkin Elmer, Calif., USA), following the manufacturer's instructions. The derived cDNA can then be used as a template in the subsequent PCR reaction.

Although the PCR step can use a variety of thermostable DNA-dependent DNA polymerases, it typically employs the Taq DNA polymerase, which has a 5′-3′ nuclease activity but lacks a 3′-5′ proofreading endonuclease activity. TaqMan PCR typically uses the 5′-nuclease activity of Taq or Tth polymerase to hydrolyze a hybridization probe bound to its target amplicon, but any enzyme with equivalent 5′ nuclease activity can be used. Two oligonucleotide primers are used to generate an amplicon typical of a PCR reaction. A third oligonucleotide, or probe, is designed to detect nucleotide sequence located between the two PCR primers. The probe is non-extendible by Taq DNA polymerase enzyme, and is labeled with a reporter fluorescent dye and a quencher fluorescent dye. Any laser-induced emission from the reporter dye is quenched by the quenching dye when the two dyes are located close together as they are on the probe. During the amplification reaction, the Taq DNA polymerase enzyme cleaves the probe in a template-dependent manner. The resultant probe fragments disassociate in solution, and signal from the released reporter dye is free from the quenching effect of the second fluorophore. One molecule of reporter dye is liberated for each new molecule synthesized, and detection of the unquenched reporter dye provides the basis for quantitative interpretation of the data.

TaqMan™ RT-PCR can be performed using commercially available equipment, such as, for example, ABI PRISM 7700™ Sequence Detection System™ (Perkin-Elmer-Applied Biosystems, Foster City, Calif., USA), or LightCycler (Roche Molecular Biochemicals, Mannheim, Germany). In one specific embodiment, the 5′ nuclease procedure is run on a real-time quantitative PCR device such as the ABI PRISM 7700 Sequence Detection System. The system consists of a thermocycler, laser, charge-coupled device (CCD), camera and computer. The system amplifies samples in a 96-well format on a thermocycler. During amplification, laser-induced fluorescent signal is collected in real-time through fiber optic cables for all 96 wells, and detected at the CCD. The system includes software for miming the instrument and for analyzing the data.

TaqMan data are initially expressed as Ct, or the threshold cycle. As discussed above, fluorescence values are recorded during every cycle and represent the amount of product amplified to that point in the amplification reaction. The point when the fluorescent signal is first recorded as statistically significant is the threshold cycle (Ct).

To minimize errors and the effect of sample-to-sample variation, RT-PCR is usually performed using an internal standard. The ideal internal standard is expressed at a constant level among different tissues, and is unaffected by the experimental treatment. RNAs most frequently used to normalize patterns of gene expression are mRNAs for the housekeeping genes glyceraldehyde-3-phosphate-dehydrogenase (GAPDH) and β-actin.

Real time quantitative PCR (also quantitative real time polymerase chain reaction, QRT-PCR or Q-PCR) is a more recent variation of the RT-PCR technique. Q-PCR can measure PCR product accumulation through a dual-labeled fluorigenic probe (i.e., TaqMan probe). Real time PCR is compatible both with quantitative competitive PCR, where internal competitor for each target sequence is used for normalization, and with quantitative comparative PCR using a normalization gene contained within the sample, or a housekeeping gene for RT-PCR. See, e.g. Held et al. (1996) Genome Research 6:986-994.

Protein-based detection techniques are also useful for molecular profiling, especially when the nucleotide variant causes amino acid substitutions or deletions or insertions or frame shift that affect the protein primary, secondary or tertiary structure. To detect the amino acid variations, protein sequencing techniques may be used. For example, a protein or fragment thereof corresponding to a gene can be synthesized by recombinant expression using a DNA fragment isolated from an individual to be tested. Preferably, a cDNA fragment of no more than 100 to 150 base pairs encompassing the polymorphic locus to be determined is used. The amino acid sequence of the peptide can then be determined by conventional protein sequencing methods. Alternatively, the HPLC-microscopy tandem mass spectrometry technique can be used for determining the amino acid sequence variations. In this technique, proteolytic digestion is performed on a protein, and the resulting peptide mixture is separated by reversed-phase chromatographic separation. Tandem mass spectrometry is then performed and the data collected is analyzed. See Gatlin et al., Anal. Chem., 72:757-763 (2000).

### Microarray

The biomarkers of the invention can also be identified, confirmed, and/or measured using the microarray technique. Thus, the expression profile biomarkers can be measured in cancer samples using microarray technology. In this method, polynucleotide sequences of interest are plated, or arrayed, on a microchip substrate. The arrayed sequences are then hybridized with specific DNA probes from cells or tissues of interest. The source of mRNA can be total RNA isolated from a sample, e.g., human tumors or tumor cell lines and corresponding normal tissues or cell lines. Thus RNA can be isolated from a variety of primary tumors or tumor cell lines. If the source of mRNA is a primary tumor, mRNA can be extracted, for example, from frozen or archived paraffin-embedded and fixed (e.g. formalin-fixed) tissue samples, which are routinely prepared and preserved in everyday clinical practice.

The expression profile of biomarkers can be measured in either fresh or paraffin-embedded tumor tissue, or body fluids using microarray technology. In this method, polynucleotide sequences of interest are plated, or arrayed, on a microchip substrate. The arrayed sequences are then hybridized with specific DNA probes from cells or tissues of interest. As with the RT-PCR method, the source of miRNA typically is total RNA isolated from human tumors or tumor cell lines, including body fluids, such as serum, urine, tears, and exosomes and corresponding normal tissues or cell lines. Thus RNA can be isolated from a variety of sources. If the source of miRNA is a primary tumor, miRNA can be extracted, for example, from frozen tissue samples, which are routinely prepared and preserved in everyday clinical practice.

Also known as biochip, DNA chip, or gene array, cDNA microarray technology allows for identification of gene expression levels in a biologic sample. cDNAs or oligonucleotides, each representing a given gene, are immobilized on a substrate, e.g., a small chip, bead or nylon membrane, tagged, and serve as probes that will indicate whether they are expressed in biologic samples of interest. The simultaneous expression of thousands of genes can be monitored simultaneously.

In a specific embodiment of the microarray technique, PCR amplified inserts of cDNA clones are applied to a substrate in a dense array. In one aspect, at least 100, 200, 300, 400, 500, 600, 700, 800, 900, 1,000, 1,500, 2,000, 3000, 4000, 5000, 6000, 7000, 8000, 9000, 10,000, 15,000, 20,000, 25,000, 30,000, 35,000, 40,000, 45,000 or at least 50,000 nucleotide sequences are applied to the substrate. Each sequence can correspond to a different gene, or multiple sequences can be arrayed per gene. The microarrayed genes, immobilized on the microchip, are suitable for hybridization under stringent conditions. Fluorescently labeled cDNA probes may be generated through incorporation of fluorescent nucleotides by reverse transcription of RNA extracted from tissues of interest. Labeled cDNA probes applied to the chip hybridize with specificity to each spot of DNA on the array. After stringent washing to remove non-specifically bound probes, the chip is scanned by confocal laser microscopy or by another detection method, such as a CCD camera. Quantitation of hybridization of each arrayed element allows for assessment of corresponding mRNA abundance. With dual color fluorescence, separately labeled cDNA probes generated from two sources of RNA are hybridized pairwise to the array. The relative abundance of the transcripts from the two sources corresponding to each specified gene is thus determined simultaneously. The miniaturized scale of the hybridization affords a convenient and rapid evaluation of the expression pattern for large numbers of genes. Such methods have been shown to have the sensitivity required to detect rare transcripts, which are expressed at a few copies per cell, and to reproducibly detect at least approximately two-fold differences in the expression levels (Schena et al. (1996) Proc. Natl. Acad. Sci. USA 93(2):106-149). Microarray analysis can be performed by commercially available equipment following manufacturer's protocols, including without limitation the Affymetrix GeneChip technology (Affymetrix, Santa Clara, Calif.), Agilent (Agilent Technologies, Inc., Santa Clara, Calif.), or Illumina (Illumina, Inc., San Diego, Calif.) microarray technology.

The development of microarray methods for large-scale analysis of gene expression makes it possible to search systematically for molecular markers of cancer classification and outcome prediction in a variety of tumor types.

In some embodiments, the Agilent Whole Human Genome Microarray Kit (Agilent Technologies, Inc., Santa Clara, Calif.). The system can analyze more than 41,000 unique human genes and transcripts represented, all with public domain annotations. The system is used according to the manufacturer's instructions.

In some embodiments, the Illumina Whole Genome DASL assay (Illumina Inc., San Diego, Calif.) is used. The system offers a method to simultaneously profile over 24,000 transcripts from minimal RNA input, from both fresh frozen (FF) and formalin-fixed paraffin embedded (FFPE) tissue sources, in a high throughput fashion.

Microarray expression analysis comprises identifying whether a gene or gene product is up-regulated or down-regulated relative to a reference. The identification can be performed using a statistical test to determine statistical significance of any differential expression observed. In some embodiments, statistical significance is determined using a parametric statistical test. The parametric statistical test can comprise, for example, a fractional factorial design, analysis of variance (ANOVA), a t-test, least squares, a Pearson correlation, simple linear regression, nonlinear regression, multiple linear regression, or multiple nonlinear regression. Alternatively, the parametric statistical test can comprise a one-way analysis of variance, two-way analysis of variance, or repeated measures analysis of variance. In other embodiments, statistical significance is determined using a nonparametric statistical test. Examples include, but are not limited to, a Wilcoxon signed-rank test, a Mann-Whitney test, a Kruskal-Wallis test, a Friedman test, a Spearman ranked order correlation coefficient, a Kendall Tau analysis, and a nonparametric regression test. In some embodiments, statistical significance is determined at a p-value of less than about 0.05, 0.01, 0.005, 0.001, 0.0005, or 0.0001. Although the microarray systems used in the methods of the invention may assay thousands of transcripts, data analysis need only be performed on the transcripts of interest, thereby reducing the problem of multiple comparisons inherent in performing multiple statistical tests. The p-values can also be corrected for multiple comparisons, e.g., using a Bonferroni correction, a modification thereof, or other technique known to those in the art, e.g., the Hochberg correction, Holm-Bonferroni correction, Šidák correction, or Dunnett's correction. The degree of differential expression can also be taken into account. For example, a gene can be considered as differentially expressed when the fold-change in expression compared to control level is at least 1.2, 1.3, 1.4, 1.5, 1.6, 1.7, 1.8, 1.9, 2.0, 2.2, 2.5, 2.7, 3.0, 4, 5, 6, 7, 8, 9 or 10-fold different in the sample versus the control. The differential expression takes into account both overexpression and underexpression. A gene or gene product can be considered up or down-regulated if the differential expression meets a statistical threshold, a fold-change threshold, or both. For example, the criteria for identifying differential expression can comprise both a p-value of 0.001 and fold change of at least 1.5-fold (up or down). One of skill will understand that such statistical and threshold measures can be adapted to determine differential expression by any molecular profiling technique disclosed herein.

Various methods of the invention make use of many types of microarrays that detect the presence and potentially the amount of biological entities in a sample. Arrays typically contain addressable moieties that can detect the presence of the entity in the sample, e.g., via a binding event. Microarrays include without limitation DNA microarrays, such as cDNA microarrays, oligonucleotide microarrays and SNP microarrays, microRNA arrays, protein microarrays, antibody microarrays, tissue microarrays, cellular microarrays (also called transfection microarrays), chemical compound microarrays, and carbohydrate arrays (glycoarrays). DNA arrays typically comprise addressable nucleotide sequences that can bind to sequences present in a sample. MicroRNA arrays, e.g., the MMChips array from the University of Louisville or commercial systems from Agilent, can be used to detect microRNAs. Protein microarrays can be used to identify protein—protein interactions, including without limitation identifying substrates of protein kinases, transcription factor protein-activation, or to identify the targets of biologically active small molecules. Protein arrays may comprise an array of different protein molecules, commonly antibodies, or nucleotide sequences that bind to proteins of interest. Antibody microarrays comprise antibodies spotted onto the protein chip that are used as capture molecules to detect proteins or other biological materials from a sample, e.g., from cell or tissue lysate solutions. For example, antibody arrays can be used to detect biomarkers from bodily fluids, e.g., serum or urine, for diagnostic applications. Tissue microarrays comprise separate tissue cores assembled in array fashion to allow multiplex histological analysis. Cellular microarrays, also called transfection microarrays, comprise various capture agents, such as antibodies, proteins, or lipids, which can interact with cells to facilitate their capture on addressable locations. Chemical compound microarrays comprise arrays of chemical compounds and can be used to detect protein or other biological materials that bind the compounds. Carbohydrate arrays (glycoarrays) comprise arrays of carbohydrates and can detect, e.g., protein that bind sugar moieties. One of skill will appreciate that similar technologies or improvements can be used according to the methods of the invention.

Certain embodiments of the current methods comprise a multi-well reaction vessel, including without limitation, a multi-well plate or a multi-chambered microfluidic device, in which a multiplicity of amplification reactions and, in some embodiments, detection are performed, typically in parallel. In certain embodiments, one or more multiplex reactions for generating amplicons are performed in the same reaction vessel, including without limitation, a multi-well plate, such as a 96-well, a 384-well, a 1536-well plate, and so forth; or a microfluidic device, for example but not limited to, a TaqMan™ Low Density Array (Applied Biosystems, Foster City, Calif.). In some embodiments, a massively parallel amplifying step comprises a multi-well reaction vessel, including a plate comprising multiple reaction wells, for example but not limited to, a 24-well plate, a 96-well plate, a 384-well plate, or a 1536-well plate; or a multi-chamber microfluidics device, for example but not limited to a low density array wherein each chamber or well comprises an appropriate primer(s), primer set(s), and/or reporter probe(s), as appropriate. Typically such amplification steps occur in a series of parallel single-plex, two-plex, three-plex, four-plex, five-plex, or six-plex reactions, although higher levels of parallel multiplexing are also within the intended scope of the current teachings. These methods can comprise PCR methodology, such as RT-PCR, in each of the wells or chambers to amplify and/or detect nucleic acid molecules of interest.

Low density arrays can include arrays that detect 10s or 100s of molecules as opposed to 1000s of molecules. These arrays can be more sensitive than high density arrays. In embodiments, a low density array such as a TaqMan™ Low Density Array is used to detect one or more gene or gene product in Table 2, Table 6 or Table 25. For example, the low density array can be used to detect at least 1, 2, 3, 4, 5, 6, 7, 8, 9, 10, 15, 20, 25, 30, 40, 50, 60, 70, 80, 90 or 100 genes or gene products in Table 2, Table 6 or Table 25.

In some embodiments, the disclosed methods comprise a microfluidics device, “lab on a chip,” or micrototal analytical system (pTAS). In some embodiments, sample preparation is performed using a microfluidics device. In some embodiments, an amplification reaction is performed using a microfluidics device. In some embodiments, a sequencing or PCR reaction is performed using a microfluidic device. In some embodiments, the nucleotide sequence of at least a part of an amplified product is obtained using a microfluidics device. In some embodiments, detecting comprises a microfluidic device, including without limitation, a low density array, such as a TaqMan™ Low Density Array. Descriptions of exemplary microfluidic devices can be found in, among other places, Published PCT Application Nos. WO/0185341 and WO 04/011666; Kartalov and Quake, Nucl. Acids Res. 32:2873-79, 2004; and Fiorini and Chiu, Bio Techniques 38:429-46, 2005.

Any appropriate microfluidic device can be used in the methods of the invention. Examples of microfluidic devices that may be used, or adapted for use with molecular profiling, include but are not limited to those described in U.S. Pat. Nos. 7,591,936, 7,581,429, 7,579,136, 7,575,722, 7,568,399, 7,552,741, 7,544,506, 7,541,578, 7,518,726, 7,488,596, 7,485,214, 7,467,928, 7,452,713, 7,452,509, 7,449,096, 7,431,887, 7,422,725, 7,422,669, 7,419,822, 7,419,639, 7,413,709, 7,411,184, 7,402,229, 7,390,463, 7,381,471, 7,357,864, 7,351,592, 7,351,380, 7,338,637, 7,329,391, 7,323,140, 7,261,824, 7,258,837, 7,253,003, 7,238,324, 7,238,255, 7,233,865, 7,229,538, 7,201,881, 7,195,986, 7,189,581, 7,189,580, 7,189,368, 7,141,978, 7,138,062, 7,135,147, 7,125,711, 7,118,910, 7,118,661, 7,640,947, 7,666,361, 7,704,735; U.S. Patent Application Publication 20060035243; and International Patent Publication WO 2010/072410; each of which patents or applications are incorporated herein by reference in their entirety. Another example for use with methods disclosed herein is described in Chen et al., “Microfluidic isolation and transcriptome analysis of serum vesicles,” Lab on a Chip, Dec. 8, 2009 DOI: 10.1039/b916199f.

Gene Expression Analysis by Massively Parallel Signature Sequencing (MPSS)

This method, described by Brenner et al. (2000) Nature Biotechnology 18:630-634, is a sequencing approach that combines non-gel-based signature sequencing with in vitro cloning of millions of templates on separate microbeads. First, a microbead library of DNA templates is constructed by in vitro cloning. This is followed by the assembly of a planar array of the template-containing microbeads in a flow cell at a high density. The free ends of the cloned templates on each microbead are analyzed simultaneously, using a fluorescence-based signature sequencing method that does not require DNA fragment separation. This method has been shown to simultaneously and accurately provide, in a single operation, hundreds of thousands of gene signature sequences from a cDNA library.

MPSS data has many uses. The expression levels of nearly all transcripts can be quantitatively determined; the abundance of signatures is representative of the expression level of the gene in the analyzed tissue. Quantitative methods for the analysis of tag frequencies and detection of differences among libraries have been published and incorporated into public databases for SAGE™ data and are applicable to MPSS data. The availability of complete genome sequences permits the direct comparison of signatures to genomic sequences and further extends the utility of MPSS data. Because the targets for MPSS analysis are not pre-selected (like on a microarray), MPSS data can characterize the full complexity of transcriptomes. This is analogous to sequencing millions of ESTs at once, and genomic sequence data can be used so that the source of the MPSS signature can be readily identified by computational means.

Serial Analysis of Gene Expression (SAGE)

Serial analysis of gene expression (SAGE) is a method that allows the simultaneous and quantitative analysis of a large number of gene transcripts, without the need of providing an individual hybridization probe for each transcript. First, a short sequence tag (e.g., about 10-14 bp) is generated that contains sufficient information to uniquely identify a transcript, provided that the tag is obtained from a unique position within each transcript. Then, many transcripts are linked together to form long serial molecules, that can be sequenced, revealing the identity of the multiple tags simultaneously. The expression pattern of any population of transcripts can be quantitatively evaluated by determining the abundance of individual tags, and identifying the gene corresponding to each tag. See, e.g. Velculescu et al. (1995) Science 270:484-487; and Velculescu et al. (1997) Cell 88:243-51.

DNA Copy Number Profiling

Any method capable of determining a DNA copy number profile of a particular sample can be used for molecular profiling according to the invention as long as the resolution is sufficient to identify the biomarkers of the invention. The skilled artisan is aware of and capable of using a number of different platforms for assessing whole genome copy number changes at a resolution sufficient to identify the copy number of the one or more biomarkers of the invention. Some of the platforms and techniques are described in the embodiments below.

In some embodiments, the copy number profile analysis involves amplification of whole genome DNA by a whole genome amplification method. The whole genome amplification method can use a strand displacing polymerase and random primers.

In some aspects of these embodiments, the copy number profile analysis involves hybridization of whole genome amplified DNA with a high density array. In a more specific aspect, the high density array has 5,000 or more different probes. In another specific aspect, the high density array has 5,000, 10,000, 20,000, 50,000, 100,000, 200,000, 300,000, 400,000, 500,000, 600,000, 700,000, 800,000, 900,000, or 1,000,000 or more different probes. In another specific aspect, each of the different probes on the array is an oligonucleotide having from about 15 to 200 bases in length. In another specific aspect, each of the different probes on the array is an oligonucleotide having from about 15 to 200, 15 to 150, 15 to 100, 15 to 75, 15 to 60, or 20 to 55 bases in length.

In some embodiments, a microarray is employed to aid in determining the copy number profile for a sample, e.g., cells from a tumor. Microarrays typically comprise a plurality of oligomers (e.g., DNA or RNA polynucleotides or oligonucleotides, or other polymers), synthesized or deposited on a substrate (e.g., glass support) in an array pattern. The support-bound oligomers are “probes”, which function to hybridize or bind with a sample material (e.g., nucleic acids prepared or obtained from the tumor samples), in hybridization experiments. The reverse situation can also be applied: the sample can be bound to the microarray substrate and the oligomer probes are in solution for the hybridization. In use, the array surface is contacted with one or more targets under conditions that promote specific, high-affinity binding of the target to one or more of the probes. In some configurations, the sample nucleic acid is labeled with a detectable label, such as a fluorescent tag, so that the hybridized sample and probes are detectable with scanning equipment. DNA array technology offers the potential of using a multitude (e.g., hundreds of thousands) of different oligonucleotides to analyze DNA copy number profiles. In some embodiments, the substrates used for arrays are surface-derivatized glass or silica, or polymer membrane surfaces (see e.g., in Z. Guo, et al., Nucleic Acids Res, 22, 5456-65 (1994); U. Maskos, E. M. Southern, Nucleic Acids Res, 20, 1679-84 (1992), and E. M. Southern, et al., Nucleic Acids Res, 22, 1368-73 (1994), each incorporated by reference herein). Modification of surfaces of array substrates can be accomplished by many techniques. For example, siliceous or metal oxide surfaces can be derivatized with bifunctional silanes, i.e., silanes having a first functional group enabling covalent binding to the surface (e.g., Si-halogen or Si-alkoxy group, as in —SiCl₃ or —Si(OCH₃)₃, respectively) and a second functional group that can impart the desired chemical and/or physical modifications to the surface to covalently or non-covalently attach ligands and/or the polymers or monomers for the biological probe array. Silylated derivatizations and other surface derivatizations that are known in the art (see for example U.S. Pat. No. 5,624,711 to Sundberg, U.S. Pat. No. 5,266,222 to Willis, and U.S. Pat. No. 5,137,765 to Farnsworth, each incorporated by reference herein). Other processes for preparing arrays are described in U.S. Pat. No. 6,649,348, to Bass et. al., assigned to Agilent Corp., which disclose DNA arrays created by in situ synthesis methods.

Polymer array synthesis is also described extensively in the literature including in the following: WO 00/58516, U.S. Pat. Nos. 5,143,854, 5,242,974, 5,252,743, 5,324,633, 5,384,261, 5,405,783, 5,424,186, 5,451,683, 5,482,867, 5,491,074, 5,527,681, 5,550,215, 5,571,639, 5,578,832, 5,593,839, 5,599,695, 5,624,711, 5,631,734, 5,795,716, 5,831,070, 5,837,832, 5,856,101, 5,858,659, 5,936,324, 5,968,740, 5,974,164, 5,981,185, 5,981,956, 6,025,601, 6,033,860, 6,040,193, 6,090,555, 6,136,269, 6,269,846 and 6,428,752, 5,412,087, 6,147,205, 6,262,216, 6,310,189, 5,889,165, and 5,959,098 in PCT Applications Nos. PCT/US99/00730 (International Publication No. WO 99/36760) and PCT/US01/04285 (International Publication No. WO 01/58593), which are all incorporated herein by reference in their entirety for all purposes.

Nucleic acid arrays that are useful in the present invention include, but are not limited to, those that are commercially available from Affymetrix (Santa Clara, Calif.) under the brand name GeneChip™. Example arrays are shown on the website at affymetrix.com. Another microarray supplier is Illumina, Inc., of San Diego, Calif. with example arrays shown on their website at illumina.com.

In some embodiments, the inventive methods provide for sample preparation. Depending on the microarray and experiment to be performed, sample nucleic acid can be prepared in a number of ways by methods known to the skilled artisan. In some aspects of the invention, prior to or concurrent with genotyping (analysis of copy number profiles), the sample may be amplified any number of mechanisms. The most common amplification procedure used involves PCR. See, for example, PCR Technology: Principles and Applications for DNA Amplification (Ed. H. A. Erlich, Freeman Press, NY, N.Y., 1992); PCR Protocols: A Guide to Methods and Applications (Eds. Innis, et al., Academic Press, San Diego, Calif., 1990); Mattila et al., Nucleic Acids Res. 19, 4967 (1991); Eckert et al., PCR Methods and Applications 1, 17 (1991); PCR (Eds. McPherson et al., IRL Press, Oxford); and U.S. Pat. Nos. 4,683,202, 4,683,195, 4,800,159 4,965,188, and 5,333,675, and each of which is incorporated herein by reference in their entireties for all purposes. In some embodiments, the sample may be amplified on the array (e.g., U.S. Pat. No. 6,300,070 which is incorporated herein by reference)

Other suitable amplification methods include the ligase chain reaction (LCR) (for example, Wu and Wallace, Genomics 4, 560 (1989), Landegren et al., Science 241, 1077 (1988) and Barringer et al. Gene 89:117 (1990)), transcription amplification (Kwoh et al., Proc. Natl. Acad. Sci. USA 86, 1173 (1989) and WO88/10315), self-sustained sequence replication (Guatelli et al., Proc. Nat. Acad. Sci. USA, 87, 1874 (1990) and WO90/06995), selective amplification of target polynucleotide sequences (U.S. Pat. No. 6,410,276), consensus sequence primed polymerase chain reaction (CP-PCR) (U.S. Pat. No. 4,437,975), arbitrarily primed polymerase chain reaction (AP-PCR) (U.S. Pat. Nos. 5,413,909, 5,861,245) and nucleic acid based sequence amplification (NABSA). (See, U.S. Pat. Nos. 5,409,818, 5,554,517, and 6,063,603, each of which is incorporated herein by reference). Other amplification methods that may be used are described in, U.S. Pat. Nos. 5,242,794, 5,494,810, 4,988,617 and in U.S. Ser. No. 09/854,317, each of which is incorporated herein by reference.

Additional methods of sample preparation and techniques for reducing the complexity of a nucleic sample are described in Dong et al., Genome Research 11, 1418 (2001), in U.S. Pat. Nos. 6,361,947, 6,391,592 and U.S. Ser. Nos. 09/916,135, 09/920,491 (U.S. Patent Application Publication 20030096235), 09/910,292 (U.S. Patent Application Publication 20030082543), and 10/013,598.

Methods for conducting polynucleotide hybridization assays are well developed in the art. Hybridization assay procedures and conditions used in the methods of the invention will vary depending on the application and are selected in accordance with the general binding methods known including those referred to in: Maniatis et al. Molecular Cloning: A Laboratory Manual (2.sup.nd Ed. Cold Spring Harbor, N.Y., 1989); Berger and Kimmel Methods in Enzymology, Vol. 152, Guide to Molecular Cloning Techniques (Academic Press, Inc., San Diego, Calif., 1987); Young and Davism, P.N.A.S, 80: 1194 (1983). Methods and apparatus for carrying out repeated and controlled hybridization reactions have been described in U.S. Pat. Nos. 5,871,928, 5,874,219, 6,045,996 and 6,386,749, 6,391,623 each of which are incorporated herein by reference.

The methods of the invention may also involve signal detection of hybridization between ligands in after (and/or during) hybridization. See U.S. Pat. Nos. 5,143,854, 5,578,832; 5,631,734; 5,834,758; 5,936,324; 5,981,956; 6,025,601; 6,141,096; 6,185,030; 6,201,639; 6,218,803; and 6,225,625, in U.S. Ser. No. 10/389,194 and in PCT Application PCT/US99/06097 (published as WO99/47964), each of which also is hereby incorporated by reference in its entirety for all purposes.

Methods and apparatus for signal detection and processing of intensity data are disclosed in, for example, U.S. Pat. Nos. 5,143,854, 5,547,839, 5,578,832, 5,631,734, 5,800,992, 5,834,758; 5,856,092, 5,902,723, 5,936,324, 5,981,956, 6,025,601, 6,090,555, 6,141,096, 6,185,030, 6,201,639; 6,218,803; and 6,225,625, in U.S. Ser. Nos. 10/389,194, 60/493,495 and in PCT Application PCT/US99/06097 (published as WO99/47964), each of which also is hereby incorporated by reference in its entirety for all purposes.

### Immuno-Based Assays

Protein-based detection molecular profiling techniques include immunoaffinity assays based on antibodies selectively immunoreactive with mutant gene encoded protein according to the present invention. These techniques include without limitation immunoprecipitation, Western blot analysis, molecular binding assays, enzyme-linked immunosorbent assay (ELISA), enzyme-linked immunofiltration assay (ELIFA), fluorescence activated cell sorting (FACS) and the like. For example, an optional method of detecting the expression of a biomarker in a sample comprises contacting the sample with an antibody against the biomarker, or an immunoreactive fragment of the antibody thereof, or a recombinant protein containing an antigen binding region of an antibody against the biomarker; and then detecting the binding of the biomarker in the sample. Methods for producing such antibodies are known in the art. Antibodies can be used to immunoprecipitate specific proteins from solution samples or to immunoblot proteins separated by, e.g., polyacrylamide gels Immunocytochemical methods can also be used in detecting specific protein polymorphisms in tissues or cells. Other well-known antibody-based techniques can also be used including, e.g., ELISA, radioimmunoassay (RIA), immunoradiometric assays (IRMA) and immunoenzymatic assays (IEMA), including sandwich assays using monoclonal or polyclonal antibodies. See, e.g., U.S. Pat. Nos. 4,376,110 and 4,486,530, both of which are incorporated herein by reference.

In alternative methods, the sample may be contacted with an antibody specific for a biomarker under conditions sufficient for an antibody-biomarker complex to form, and then detecting said complex. The presence of the biomarker may be detected in a number of ways, such as by Western blotting and ELISA procedures for assaying a wide variety of tissues and samples, including plasma or serum. A wide range of immunoassay techniques using such an assay format are available, see, e.g., U.S. Pat. Nos. 4,016,043, 4,424,279 and 4,018,653. These include both single-site and two-site or “sandwich” assays of the non-competitive types, as well as in the traditional competitive binding assays. These assays also include direct binding of a labelled antibody to a target biomarker.

A number of variations of the sandwich assay technique exist, and all are intended to be encompassed by the present invention. Briefly, in a typical forward assay, an unlabelled antibody is immobilized on a solid substrate, and the sample to be tested brought into contact with the bound molecule. After a suitable period of incubation, for a period of time sufficient to allow formation of an antibody-antigen complex, a second antibody specific to the antigen, labelled with a reporter molecule capable of producing a detectable signal is then added and incubated, allowing time sufficient for the formation of another complex of antibody-antigen-labelled antibody. Any unreacted material is washed away, and the presence of the antigen is determined by observation of a signal produced by the reporter molecule. The results may either be qualitative, by simple observation of the visible signal, or may be quantitated by comparing with a control sample containing known amounts of biomarker.

Variations on the forward assay include a simultaneous assay, in which both sample and labelled antibody are added simultaneously to the bound antibody. These techniques are well known to those skilled in the art, including any minor variations as will be readily apparent. In a typical forward sandwich assay, a first antibody having specificity for the biomarker is either covalently or passively bound to a solid surface. The solid surface is typically glass or a polymer, the most commonly used polymers being cellulose, polyacrylamide, nylon, polystyrene, polyvinyl chloride or polypropylene. The solid supports may be in the form of tubes, beads, discs of microplates, or any other surface suitable for conducting an immunoassay. The binding processes are well-known in the art and generally consist of cross-linking covalently binding or physically adsorbing, the polymer-antibody complex is washed in preparation for the test sample. An aliquot of the sample to be tested is then added to the solid phase complex and incubated for a period of time sufficient (e.g. 2-40 minutes or overnight if more convenient) and under suitable conditions (e.g. from room temperature to 40° C. such as between 25° C. and 32° C. inclusive) to allow binding of any subunit present in the antibody. Following the incubation period, the antibody subunit solid phase is washed and dried and incubated with a second antibody specific for a portion of the biomarker. The second antibody is linked to a reporter molecule which is used to indicate the binding of the second antibody to the molecular marker.

An alternative method involves immobilizing the target biomarkers in the sample and then exposing the immobilized target to specific antibody which may or may not be labelled with a reporter molecule. Depending on the amount of target and the strength of the reporter molecule signal, a bound target may be detectable by direct labelling with the antibody. Alternatively, a second labelled antibody, specific to the first antibody is exposed to the target-first antibody complex to form a target-first antibody-second antibody tertiary complex. The complex is detected by the signal emitted by the reporter molecule. By “reporter molecule”, as used in the present specification, is meant a molecule which, by its chemical nature, provides an analytically identifiable signal which allows the detection of antigen-bound antibody. The most commonly used reporter molecules in this type of assay are either enzymes, fluorophores or radionuclide containing molecules (i.e. radioisotopes) and chemiluminescent molecules.

In the case of an enzyme immunoassay, an enzyme is conjugated to the second antibody, generally by means of glutaraldehyde or periodate. As will be readily recognized, however, a wide variety of different conjugation techniques exist, which are readily available to the skilled artisan. Commonly used enzymes include horseradish peroxidase, glucose oxidase, β-galactosidase and alkaline phosphatase, amongst others. The substrates to be used with the specific enzymes are generally chosen for the production, upon hydrolysis by the corresponding enzyme, of a detectable color change. Examples of suitable enzymes include alkaline phosphatase and peroxidase. It is also possible to employ fluorogenic substrates, which yield a fluorescent product rather than the chromogenic substrates noted above. In all cases, the enzyme-labelled antibody is added to the first antibody-molecular marker complex, allowed to bind, and then the excess reagent is washed away. A solution containing the appropriate substrate is then added to the complex of antibody-antigen-antibody. The substrate will react with the enzyme linked to the second antibody, giving a qualitative visual signal, which may be further quantitated, usually spectrophotometrically, to give an indication of the amount of biomarker which was present in the sample. Alternately, fluorescent compounds, such as fluorescein and rhodamine, may be chemically coupled to antibodies without altering their binding capacity. When activated by illumination with light of a particular wavelength, the fluorochrome-labelled antibody adsorbs the light energy, inducing a state to excitability in the molecule, followed by emission of the light at a characteristic color visually detectable with a light microscope. As in the EIA, the fluorescent labelled antibody is allowed to bind to the first antibody-molecular marker complex. After washing off the unbound reagent, the remaining tertiary complex is then exposed to the light of the appropriate wavelength, the fluorescence observed indicates the presence of the molecular marker of interest Immunofluorescence and EIA techniques are both very well established in the art. However, other reporter molecules, such as radioisotope, chemiluminescent or bioluminescent molecules, may also be employed.

Immunohistochemistry (IHC)

IHC is a process of localizing antigens (e.g., proteins) in cells of a tissue binding antibodies specifically to antigens in the tissues. The antigen-binding antibody can be conjugated or fused to a tag that allows its detection, e.g., via visualization. In some embodiments, the tag is an enzyme that can catalyze a color-producing reaction, such as alkaline phosphatase or horseradish peroxidase. The enzyme can be fused to the antibody or non-covalently bound, e.g., using a biotin-avadin system. Alternatively, the antibody can be tagged with a fluorophore, such as fluorescein, rhodamine, DyLight Fluor or Alexa Fluor. The antigen-binding antibody can be directly tagged or it can itself be recognized by a detection antibody that carries the tag. Using IHC, one or more proteins may be detected. The expression of a gene product can be related to its staining intensity compared to control levels. In some embodiments, the gene product is considered differentially expressed if its staining varies at least 1.2, 1.3, 1.4, 1.5, 1.6, 1.7, 1.8, 1.9, 2.0, 2.2, 2.5, 2.7, 3.0, 4, 5, 6, 7, 8, 9 or 10-fold in the sample versus the control.

IHC comprises the application of antigen-antibody interactions to histochemical techniques. In an illustrative example, a tissue section is mounted on a slide and is incubated with antibodies (polyclonal or monoclonal) specific to the antigen (primary reaction). The antigen-antibody signal is then amplified using a second antibody conjugated to a complex of peroxidase antiperoxidase (PAP), avidin-biotin-peroxidase (ABC) or avidin-biotin alkaline phosphatase. In the presence of substrate and chromogen, the enzyme forms a colored deposit at the sites of antibody-antigen binding Immunofluorescence is an alternate approach to visualize antigens. In this technique, the primary antigen-antibody signal is amplified using a second antibody conjugated to a fluorochrome. On UV light absorption, the fluorochrome emits its own light at a longer wavelength (fluorescence), thus allowing localization of antibody-antigen complexes.

### Epigenetic Status

Molecular profiling methods according to the invention also comprise measuring epigenetic change, i.e., modification in a gene caused by an epigenetic mechanism, such as a change in methylation status or histone acetylation. Frequently, the epigenetic change will result in an alteration in the levels of expression of the gene which may be detected (at the RNA or protein level as appropriate) as an indication of the epigenetic change. Often the epigenetic change results in silencing or down regulation of the gene, referred to as “epigenetic silencing.” The most frequently investigated epigenetic change in the methods of the invention involves determining the DNA methylation status of a gene, where an increased level of methylation is typically associated with the relevant cancer (since it may cause down regulation of gene expression). Aberrant methylation, which may be referred to as hypermethylation, of the gene or genes can be detected. Typically, the methylation status is determined in suitable CpG islands which are often found in the promoter region of the gene(s). The term “methylation,” “methylation state” or “methylation status” may refers to the presence or absence of 5-methylcytosine at one or a plurality of CpG dinucleotides within a DNA sequence. CpG dinucleotides are typically concentrated in the promoter regions and exons of human genes.

Diminished gene expression can be assessed in terms of DNA methylation status or in terms of expression levels as determined by the methylation status of the gene. One method to detect epigenetic silencing is to determine that a gene which is expressed in normal cells is less expressed or not expressed in tumor cells. Accordingly, the invention provides for a method of molecular profiling comprising detecting epigenetic silencing.

Various assay procedures to directly detect methylation are known in the art, and can be used in conjunction with the present invention. These assays rely onto two distinct approaches: bisulphite conversion based approaches and non-bisulphite based approaches. Non-bisulphite based methods for analysis of DNA methylation rely on the inability of methylation-sensitive enzymes to cleave methylation cytosines in their restriction. The bisulphite conversion relies on treatment of DNA samples with sodium bisulphite which converts unmethylated cytosine to uracil, while methylated cytosines are maintained (Furuichi Y, Wataya Y, Hayatsu H, Ukita T. Biochem Biophys Res Commun. 1970 Dec. 9; 41(5):1185-91). This conversion results in a change in the sequence of the original DNA. Methods to detect such changes include MS AP-PCR (Methylation-Sensitive Arbitrarily-Primed Polymerase Chain Reaction), a technology that allows for a global scan of the genome using CG-rich primers to focus on the regions most likely to contain CpG dinucleotides, and described by Gonzalgo et al., Cancer Research 57:594-599, 1997; MethyLight™, which refers to the art-recognized fluorescence-based real-time PCR technique described by Eads et al., Cancer Res. 59:2302-2306, 1999; the HeavyMethylTMassay, in the embodiment thereof implemented herein, is an assay, wherein methylation specific blocking probes (also referred to herein as blockers) covering CpG positions between, or covered by the amplification primers enable methylation-specific selective amplification of a nucleic acid sample;

HeavyMethylTMMethyLight™ is a variation of the MethyLight™ assay wherein the MethyLight™ assay is combined with methylation specific blocking probes covering CpG positions between the amplification primers; Ms-SNuPE (Methylation-sensitive Single Nucleotide Primer Extension) is an assay described by Gonzalgo & Jones, Nucleic Acids Res. 25:2529-2531, 1997; MSP (Methylation-specific PCR) is a methylation assay described by Herman et al. Proc. Natl. Acad. Sci. USA 93:9821-9826, 1996, and by U.S. Pat. No. 5,786,146; COBRA (Combined Bisulfite Restriction Analysis) is a methylation assay described by Xiong & Laird, Nucleic Acids Res. 25:2532-2534, 1997; MCA (Methylated CpG Island Amplification) is a methylation assay described by Toyota et al., Cancer Res. 59:2307-12, 1999, and in WO 00/26401A1.

Other techniques for DNA methylation analysis include sequencing, methylation-specific PCR (MS-PCR), melting curve methylation-specific PCR (McMS-PCR), MLPA with or without bisulfite treatment, QAMA, MSRE-PCR, MethyLight, ConLight-MSP, bisulfite conversion-specific methylation-specific PCR (BS-MSP), COBRA (which relies upon use of restriction enzymes to reveal methylation dependent sequence differences in PCR products of sodium bisulfite-treated DNA), methylation-sensitive single-nucleotide primer extension conformation (MS-SNuPE), methylation-sensitive single-strand conformation analysis (MS-SSCA), Melting curve combined bisulfite restriction analysis (McCOBRA), PyroMethA, HeavyMethyl, MALDI-TOF, MassARRAY, Quantitative analysis of methylated alleles (QAMA), enzymatic regional methylation assay (ERMA), QBSUPT, MethylQuant, Quantitative PCR sequencing and oligonucleotide-based microarray systems, Pyrosequencing, Meth-DOP-PCR. A review of some useful techniques is provided in Nucleic acids research, 1998, Vol. 26, No. 10, 2255-2264; Nature Reviews, 2003, Vol. 3, 253-266; Oral Oncology, 2006, Vol. 42, 5-13, which references are incorporated herein in their entirety. Any of these techniques may be used in accordance with the present invention, as appropriate. Other techniques are described in U.S. Patent Publications 20100144836; and 20100184027, which applications are incorporated herein by reference in their entirety.

Through the activity of various acetylases and deacetylylases the DNA binding function of histone proteins is tightly regulated. Furthermore, histone acetylation and histone deactelyation have been linked with malignant progression. See Nature, 429: 457-63, 2004. Methods to analyze histone acetylation are described in U.S. Patent Publications 20100144543 and 20100151468, which applications are incorporated herein by reference in their entirety.

### Sequence Analysis

Molecular profiling according to the present invention comprises methods for genotyping one or more biomarkers by determining whether an individual has one or more nucleotide variants (or amino acid variants) in one or more of the genes or gene products. Genotyping one or more genes according to the methods of the invention in some embodiments, can provide more evidence for selecting a treatment.

The biomarkers of the invention can be analyzed by any method useful for determining alterations in nucleic acids or the proteins they encode. According to one embodiment, the ordinary skilled artisan can analyze the one or more genes for mutations including deletion mutants, insertion mutants, frame shift mutants, nonsense mutants, missense mutant, and splice mutants.

Nucleic acid used for analysis of the one or more genes can be isolated from cells in the sample according to standard methodologies (Sambrook et al., 1989). The nucleic acid, for example, may be genomic DNA or fractionated or whole cell RNA, or miRNA acquired from exosomes or cell surfaces. Where RNA is used, it may be desired to convert the RNA to a complementary DNA. In one embodiment, the RNA is whole cell RNA; in another, it is poly-A RNA; in another, it is exosomal RNA. Normally, the nucleic acid is amplified. Depending on the format of the assay for analyzing the one or more genes, the specific nucleic acid of interest is identified in the sample directly using amplification or with a second, known nucleic acid following amplification. Next, the identified product is detected. In certain applications, the detection may be performed by visual means (e.g., ethidium bromide staining of a gel). Alternatively, the detection may involve indirect identification of the product via chemiluminescence, radioactive scintigraphy of radiolabel or fluorescent label or even via a system using electrical or thermal impulse signals (Affymax Technology; Bellus, 1994).

Various types of defects are known to occur in the biomarkers of the invention. Alterations include without limitation deletions, insertions, point mutations, and duplications. Point mutations can be silent or can result in stop codons, frame shift mutations or amino acid substitutions. Mutations in and outside the coding region of the one or more genes may occur and can be analyzed according to the methods of the invention. The target site of a nucleic acid of interest can include the region wherein the sequence varies. Examples include, but are not limited to, polymorphisms which exist in different forms such as single nucleotide variations, nucleotide repeats, multibase deletion (more than one nucleotide deleted from the consensus sequence), multibase insertion (more than one nucleotide inserted from the consensus sequence), microsatellite repeats (small numbers of nucleotide repeats with a typical 5-1000 repeat units), di-nucleotide repeats, tri-nucleotide repeats, sequence rearrangements (including translocation and duplication), chimeric sequence (two sequences from different gene origins are fused together), and the like. Among sequence polymorphisms, the most frequent polymorphisms in the human genome are single-base variations, also called single-nucleotide polymorphisms (SNPs). SNPs are abundant, stable and widely distributed across the genome.

Molecular profiling includes methods for haplotyping one or more genes. The haplotype is a set of genetic determinants located on a single chromosome and it typically contains a particular combination of alleles (all the alternative sequences of a gene) in a region of a chromosome. In other words, the haplotype is phased sequence information on individual chromosomes. Very often, phased SNPs on a chromosome define a haplotype. A combination of haplotypes on chromosomes can determine a genetic profile of a cell. It is the haplotype that determines a linkage between a specific genetic marker and a disease mutation. Haplotyping can be done by any methods known in the art. Common methods of scoring SNPs include hybridization microarray or direct gel sequencing, reviewed in Landgren et al., Genome Research, 8:769-776, 1998. For example, only one copy of one or more genes can be isolated from an individual and the nucleotide at each of the variant positions is determined. Alternatively, an allele specific PCR or a similar method can be used to amplify only one copy of the one or more genes in an individual, and the SNPs at the variant positions of the present invention are determined. The Clark method known in the art can also be employed for haplotyping. A high throughput molecular haplotyping method is also disclosed in Tost et al., Nucleic Acids Res., 30(19):e96 (2002), which is incorporated herein by reference.

Thus, additional variant(s) that are in linkage disequilibrium with the variants and/or haplotypes of the present invention can be identified by a haplotyping method known in the art, as will be apparent to a skilled artisan in the field of genetics and haplotyping. The additional variants that are in linkage disequilibrium with a variant or haplotype of the present invention can also be useful in the various applications as described below.

For purposes of genotyping and haplotyping, both genomic DNA and mRNA/cDNA can be used, and both are herein referred to generically as “gene.”

Numerous techniques for detecting nucleotide variants are known in the art and can all be used for the method of this invention. The techniques can be protein-based or nucleic acid-based. In either case, the techniques used must be sufficiently sensitive so as to accurately detect the small nucleotide or amino acid variations. Very often, a probe is used which is labeled with a detectable marker. Unless otherwise specified in a particular technique described below, any suitable marker known in the art can be used, including but not limited to, radioactive isotopes, fluorescent compounds, biotin which is detectable using streptavidin, enzymes (e.g., alkaline phosphatase), substrates of an enzyme, ligands and antibodies, etc. See Jablonski et al., Nucleic Acids Res., 14:6115-6128 (1986); Nguyen et al., Biotechniques, 13:116-123 (1992); Rigby et al., J. Mol. Biol., 113:237-251 (1977).

In a nucleic acid-based detection method, target DNA sample, i.e., a sample containing genomic DNA, cDNA, mRNA and/or miRNA, corresponding to the one or more genes must be obtained from the individual to be tested. Any tissue or cell sample containing the genomic DNA, miRNA, mRNA, and/or cDNA (or a portion thereof) corresponding to the one or more genes can be used. For this purpose, a tissue sample containing cell nucleus and thus genomic DNA can be obtained from the individual. Blood samples can also be useful except that only white blood cells and other lymphocytes have cell nucleus, while red blood cells are without a nucleus and contain only mRNA or miRNA. Nevertheless, miRNA and mRNA are also useful as either can be analyzed for the presence of nucleotide variants in its sequence or serve as template for cDNA synthesis. The tissue or cell samples can be analyzed directly without much processing. Alternatively, nucleic acids including the target sequence can be extracted, purified, and/or amplified before they are subject to the various detecting procedures discussed below. Other than tissue or cell samples, cDNAs or genomic DNAs from a cDNA or genomic DNA library constructed using a tissue or cell sample obtained from the individual to be tested are also useful.

To determine the presence or absence of a particular nucleotide variant, sequencing of the target genomic DNA or cDNA, particularly the region encompassing the nucleotide variant locus to be detected. Various sequencing techniques are generally known and widely used in the art including the Sanger method and Gilbert chemical method. The pyrosequencing method monitors DNA synthesis in real time using a luminometric detection system. Pyrosequencing has been shown to be effective in analyzing genetic polymorphisms such as single-nucleotide polymorphisms and can also be used in the present invention. See Nordstrom et al., Bioteehnol. Appl. Biochem., 31(2):107-112 (2000); Ahmadian et al., Anal. Biochem., 280:103-110 (2000).

Nucleic acid variants can be detected by a suitable detection process. Non limiting examples of methods of detection, quantification, sequencing and the like are; mass detection of mass modified amplicons (e.g., matrix-assisted laser desorption ionization (MALDI) mass spectrometry and electrospray (ES) mass spectrometry), a primer extension method (e.g., iPLEX™; Sequenom, Inc.), microsequencing methods (e.g., a modification of primer extension methodology), ligase sequence determination methods (e.g., U.S. Pat. Nos. 5,679,524 and 5,952,174, and WO 01/27326), mismatch sequence determination methods (e.g., U.S. Pat. Nos. 5,851,770; 5,958,692; 6,110,684; and 6,183,958), direct DNA sequencing, fragment analysis (FA), restriction fragment length polymorphism (RFLP analysis), allele specific oligonucleotide (ASO) analysis, methylation-specific PCR (MSPCR), pyrosequencing analysis, acycloprime analysis, Reverse dot blot, GeneChip microarrays, Dynamic allele-specific hybridization (DASH), Peptide nucleic acid (PNA) and locked nucleic acids (LNA) probes, TaqMan, Molecular Beacons, Intercalating dye, FRET primers, AlphaScreen, SNPstream, genetic bit analysis (GBA), Multiplex minisequencing, SNaPshot, GOOD assay, Microarray miniseq, arrayed primer extension (APEX), Microarray primer extension (e.g., microarray sequence determination methods), Tag arrays, Coded microspheres, Template-directed incorporation (TDI), fluorescence polarization, Colorimetric oligonucleotide ligation assay (OLA), Sequence-coded OLA, Microarray ligation, Ligase chain reaction, Padlock probes, Invader assay, hybridization methods (e.g., hybridization using at least one probe, hybridization using at least one fluorescently labeled probe, and the like), conventional dot blot analyses, single strand conformational polymorphism analysis (SSCP, e.g., U.S. Pat. Nos. 5,891,625 and 6,013,499; Orita et al., Proc. Natl. Acad. Sci. U.S.A. 86: 27776-2770 (1989)), denaturing gradient gel electrophoresis (DGGE), heteroduplex analysis, mismatch cleavage detection, and techniques described in Sheffield et al., Proc. Natl. Acad. Sci. USA 49: 699-706 (1991), White et al., Genomics 12: 301-306 (1992), Grompe et al., Proc. Natl. Acad. Sci. USA 86: 5855-5892 (1989), and Grompe, Nature Genetics 5: 111-117 (1993), cloning and sequencing, electrophoresis, the use of hybridization probes and quantitative real time polymerase chain reaction (QRT-PCR), digital PCR, nanopore sequencing, chips and combinations thereof. The detection and quantification of alleles or paralogs can be carried out using the “closed-tube” methods described in U.S. patent application Ser. No. 11/950,395, filed on Dec. 4, 2007. In some embodiments the amount of a nucleic acid species is determined by mass spectrometry, primer extension, sequencing (e.g., any suitable method, for example nanopore or pyrosequencing), Quantitative PCR (Q-PCR or QRT-PCR), digital PCR, combinations thereof, and the like.

The term “sequence analysis” as used herein refers to determining a nucleotide sequence, e.g., that of an amplification product. The entire sequence or a partial sequence of a polynucleotide, e.g., DNA or mRNA, can be determined, and the determined nucleotide sequence can be referred to as a “read” or “sequence read.” For example, linear amplification products may be analyzed directly without further amplification in some embodiments (e.g., by using single-molecule sequencing methodology). In certain embodiments, linear amplification products may be subject to further amplification and then analyzed (e.g., using sequencing by ligation or pyrosequencing methodology). Reads may be subject to different types of sequence analysis. Any suitable sequencing method can be used to detect, and determine the amount of, nucleotide sequence species, amplified nucleic acid species, or detectable products generated from the foregoing. Examples of certain sequencing methods are described hereafter.

A sequence analysis apparatus or sequence analysis component(s) includes an apparatus, and one or more components used in conjunction with such apparatus, that can be used by a person of ordinary skill to determine a nucleotide sequence resulting from processes described herein (e.g., linear and/or exponential amplification products). Examples of sequencing platforms include, without limitation, the 454 platform (Roche) (Margulies, M. et al. 2005 Nature 437, 376-380), Illumina Genomic Analyzer (or Solexa platform) or SOLID System (Applied Biosystems; see PCT patent application publications WO 06/084132 entitled “Reagents, Methods, and Libraries For Bead-Based Sequencing” and WO07/121,489 entitled “Reagents, Methods, and Libraries for Gel-Free Bead-Based Sequencing”), the Helicos True Single Molecule DNA sequencing technology (Harris T D et al. 2008 Science, 320, 106-109), the single molecule, real-time (SMRT™) technology of Pacific Biosciences, and nanopore sequencing (Soni G V and Meller A. 2007 Clin Chem 53: 1996-2001), Ion semiconductor sequencing (Ion Torrent Systems, Inc, San Francisco, Calif.), or DNA nanoball sequencing (Complete Genomics, Mountain View, Calif.), VisiGen Biotechnologies approach (Invitrogen) and polony sequencing. Such platforms allow sequencing of many nucleic acid molecules isolated from a specimen at high orders of multiplexing in a parallel manner (Dear Brief Funct Genomic Proteomic 2003; 1: 397-416; Haimovich, Methods, challenges, and promise of next-generation sequencing in cancer biology. Yale J Biol Med. 2011 December; 84(4):439-46). These non-Sanger-based sequencing technologies are sometimes referred to as NextGen sequencing, NGS, next-generation sequencing, next generation sequencing, and variations thereof. Typically they allow much higher throughput than the traditional Sanger approach. See Schuster, Next-generation sequencing transforms today's biology, Nature Methods 5:16-18 (2008); Metzker, Sequencing technologies—the next generation. Nat Rev Genet. 2010 January; 11(1):31-46. These platforms can allow sequencing of clonally expanded or non-amplified single molecules of nucleic acid fragments. Certain platforms involve, for example, sequencing by ligation of dye-modified probes (including cyclic ligation and cleavage), pyrosequencing, and single-molecule sequencing. Nucleotide sequence species, amplification nucleic acid species and detectable products generated there from can be analyzed by such sequence analysis platforms. Next-generation sequencing can be used in the methods of the invention, e.g., to determine mutations, copy number, or expression levels, as appropriate. The methods can be used to perform whole genome sequencing or sequencing of specific sequences of interest, such as a gene of interest or a fragment thereof.

Sequencing by ligation is a nucleic acid sequencing method that relies on the sensitivity of DNA ligase to base-pairing mismatch. DNA ligase joins together ends of DNA that are correctly base paired. Combining the ability of DNA ligase to join together only correctly base paired DNA ends, with mixed pools of fluorescently labeled oligonucleotides or primers, enables sequence determination by fluorescence detection. Longer sequence reads may be obtained by including primers containing cleavable linkages that can be cleaved after label identification. Cleavage at the linker removes the label and regenerates the 5′ phosphate on the end of the ligated primer, preparing the primer for another round of ligation. In some embodiments primers may be labeled with more than one fluorescent label, e.g., at least 1, 2, 3, 4, or 5 fluorescent labels.

Sequencing by ligation generally involves the following steps. Clonal bead populations can be prepared in emulsion microreactors containing target nucleic acid template sequences, amplification reaction components, beads and primers. After amplification, templates are denatured and bead enrichment is performed to separate beads with extended templates from undesired beads (e.g., beads with no extended templates). The template on the selected beads undergoes a 3′ modification to allow covalent bonding to the slide, and modified beads can be deposited onto a glass slide. Deposition chambers offer the ability to segment a slide into one, four or eight chambers during the bead loading process. For sequence analysis, primers hybridize to the adapter sequence. A set of four color dye-labeled probes competes for ligation to the sequencing primer. Specificity of probe ligation is achieved by interrogating every 4th and 5th base during the ligation series. Five to seven rounds of ligation, detection and cleavage record the color at every 5th position with the number of rounds determined by the type of library used. Following each round of ligation, a new complimentary primer offset by one base in the 5′ direction is laid down for another series of ligations. Primer reset and ligation rounds (5-7 ligation cycles per round) are repeated sequentially five times to generate 25-35 base pairs of sequence for a single tag. With mate-paired sequencing, this process is repeated for a second tag.

Pyrosequencing is a nucleic acid sequencing method based on sequencing by synthesis, which relies on detection of a pyrophosphate released on nucleotide incorporation. Generally, sequencing by synthesis involves synthesizing, one nucleotide at a time, a DNA strand complimentary to the strand whose sequence is being sought. Target nucleic acids may be immobilized to a solid support, hybridized with a sequencing primer, incubated with DNA polymerase, ATP sulfurylase, luciferase, apyrase, adenosine 5′ phosphosulfate and luciferin. Nucleotide solutions are sequentially added and removed. Correct incorporation of a nucleotide releases a pyrophosphate, which interacts with ATP sulfurylase and produces ATP in the presence of adenosine 5′ phosphosulfate, fueling the luciferin reaction, which produces a chemiluminescent signal allowing sequence determination. The amount of light generated is proportional to the number of bases added. Accordingly, the sequence downstream of the sequencing primer can be determined. An illustrative system for pyrosequencing involves the following steps: ligating an adaptor nucleic acid to a nucleic acid under investigation and hybridizing the resulting nucleic acid to a bead; amplifying a nucleotide sequence in an emulsion; sorting beads using a picoliter multiwell solid support; and sequencing amplified nucleotide sequences by pyrosequencing methodology (e.g., Nakano et al., “Single-molecule PCR using water-in-oil emulsion;” Journal of Biotechnology 102: 117-124 (2003)).

Certain single-molecule sequencing embodiments are based on the principal of sequencing by synthesis, and use single-pair Fluorescence Resonance Energy Transfer (single pair FRET) as a mechanism by which photons are emitted as a result of successful nucleotide incorporation. The emitted photons often are detected using intensified or high sensitivity cooled charge-couple-devices in conjunction with total internal reflection microscopy (TIRM). Photons are only emitted when the introduced reaction solution contains the correct nucleotide for incorporation into the growing nucleic acid chain that is synthesized as a result of the sequencing process. In FRET based single-molecule sequencing, energy is transferred between two fluorescent dyes, sometimes polymethine cyanine dyes Cy3 and Cy5, through long-range dipole interactions. The donor is excited at its specific excitation wavelength and the excited state energy is transferred, non-radiatively to the acceptor dye, which in turn becomes excited. The acceptor dye eventually returns to the ground state by radiative emission of a photon. The two dyes used in the energy transfer process represent the “single pair” in single pair FRET. Cy3 often is used as the donor fluorophore and often is incorporated as the first labeled nucleotide. Cy5 often is used as the acceptor fluorophore and is used as the nucleotide label for successive nucleotide additions after incorporation of a first Cy3 labeled nucleotide. The fluorophores generally are within 10 nanometers of each for energy transfer to occur successfully.

An example of a system that can be used based on single-molecule sequencing generally involves hybridizing a primer to a target nucleic acid sequence to generate a complex; associating the complex with a solid phase; iteratively extending the primer by a nucleotide tagged with a fluorescent molecule; and capturing an image of fluorescence resonance energy transfer signals after each iteration (e.g., U.S. Pat. No. 7,169,314; Braslaysky et al., PNAS 100(7): 3960-3964 (2003)). Such a system can be used to directly sequence amplification products (linearly or exponentially amplified products) generated by processes described herein. In some embodiments the amplification products can be hybridized to a primer that contains sequences complementary to immobilized capture sequences present on a solid support, a bead or glass slide for example. Hybridization of the primer-amplification product complexes with the immobilized capture sequences, immobilizes amplification products to solid supports for single pair FRET based sequencing by synthesis. The primer often is fluorescent, so that an initial reference image of the surface of the slide with immobilized nucleic acids can be generated. The initial reference image is useful for determining locations at which true nucleotide incorporation is occurring. Fluorescence signals detected in array locations not initially identified in the “primer only” reference image are discarded as non-specific fluorescence. Following immobilization of the primer-amplification product complexes, the bound nucleic acids often are sequenced in parallel by the iterative steps of, a) polymerase extension in the presence of one fluorescently labeled nucleotide, b) detection of fluorescence using appropriate microscopy, TIRM for example, c) removal of fluorescent nucleotide, and d) return to step a with a different fluorescently labeled nucleotide.

In some embodiments, nucleotide sequencing may be by solid phase single nucleotide sequencing methods and processes. Solid phase single nucleotide sequencing methods involve contacting target nucleic acid and solid support under conditions in which a single molecule of sample nucleic acid hybridizes to a single molecule of a solid support. Such conditions can include providing the solid support molecules and a single molecule of target nucleic acid in a “microreactor.” Such conditions also can include providing a mixture in which the target nucleic acid molecule can hybridize to solid phase nucleic acid on the solid support. Single nucleotide sequencing methods useful in the embodiments described herein are described in U.S. Provisional Patent Application Ser. No. 61/021,871 filed Jan. 17, 2008.

In certain embodiments, nanopore sequencing detection methods include (a) contacting a target nucleic acid for sequencing (“base nucleic acid,” e.g., linked probe molecule) with sequence-specific detectors, under conditions in which the detectors specifically hybridize to substantially complementary subsequences of the base nucleic acid; (b) detecting signals from the detectors and (c) determining the sequence of the base nucleic acid according to the signals detected. In certain embodiments, the detectors hybridized to the base nucleic acid are disassociated from the base nucleic acid (e.g., sequentially dissociated) when the detectors interfere with a nanopore structure as the base nucleic acid passes through a pore, and the detectors disassociated from the base sequence are detected. In some embodiments, a detector disassociated from a base nucleic acid emits a detectable signal, and the detector hybridized to the base nucleic acid emits a different detectable signal or no detectable signal. In certain embodiments, nucleotides in a nucleic acid (e.g., linked probe molecule) are substituted with specific nucleotide sequences corresponding to specific nucleotides (“nucleotide representatives”), thereby giving rise to an expanded nucleic acid (e.g., U.S. Pat. No. 6,723,513), and the detectors hybridize to the nucleotide representatives in the expanded nucleic acid, which serves as a base nucleic acid. In such embodiments, nucleotide representatives may be arranged in a binary or higher order arrangement (e.g., Soni and Meller, Clinical Chemistry 53(11): 1996-2001 (2007)). In some embodiments, a nucleic acid is not expanded, does not give rise to an expanded nucleic acid, and directly serves a base nucleic acid (e.g., a linked probe molecule serves as a non-expanded base nucleic acid), and detectors are directly contacted with the base nucleic acid. For example, a first detector may hybridize to a first subsequence and a second detector may hybridize to a second subsequence, where the first detector and second detector each have detectable labels that can be distinguished from one another, and where the signals from the first detector and second detector can be distinguished from one another when the detectors are disassociated from the base nucleic acid. In certain embodiments, detectors include a region that hybridizes to the base nucleic acid (e.g., two regions), which can be about 3 to about 100 nucleotides in length (e.g., about 4, 5, 6, 7, 8, 9, 10, 11, 12, 13, 14, 15, 16, 17, 18, 19, 20, 25, 30, 35, 40, 50, 55, 60, 65, 70, 75, 80, 85, 90, or 95 nucleotides in length). A detector also may include one or more regions of nucleotides that do not hybridize to the base nucleic acid. In some embodiments, a detector is a molecular beacon. A detector often comprises one or more detectable labels independently selected from those described herein. Each detectable label can be detected by any convenient detection process capable of detecting a signal generated by each label (e.g., magnetic, electric, chemical, optical and the like). For example, a CD camera can be used to detect signals from one or more distinguishable quantum dots linked to a detector.

In certain sequence analysis embodiments, reads may be used to construct a larger nucleotide sequence, which can be facilitated by identifying overlapping sequences in different reads and by using identification sequences in the reads. Such sequence analysis methods and software for constructing larger sequences from reads are known to the person of ordinary skill (e.g., Venter et al., Science 291: 1304-1351 (2001)). Specific reads, partial nucleotide sequence constructs, and full nucleotide sequence constructs may be compared between nucleotide sequences within a sample nucleic acid (i.e., internal comparison) or may be compared with a reference sequence (i.e., reference comparison) in certain sequence analysis embodiments. Internal comparisons can be performed in situations where a sample nucleic acid is prepared from multiple samples or from a single sample source that contains sequence variations. Reference comparisons sometimes are performed when a reference nucleotide sequence is known and an objective is to determine whether a sample nucleic acid contains a nucleotide sequence that is substantially similar or the same, or different, than a reference nucleotide sequence. Sequence analysis can be facilitated by the use of sequence analysis apparatus and components described above.

Primer extension polymorphism detection methods, also referred to herein as “microsequencing” methods, typically are carried out by hybridizing a complementary oligonucleotide to a nucleic acid carrying the polymorphic site. In these methods, the oligonucleotide typically hybridizes adjacent to the polymorphic site. The term “adjacent” as used in reference to “microsequencing” methods, refers to the 3′ end of the extension oligonucleotide being sometimes 1 nucleotide from the 5′ end of the polymorphic site, often 2 or 3, and at times 4, 5, 6, 7, 8, 9, or 10 nucleotides from the 5′ end of the polymorphic site, in the nucleic acid when the extension oligonucleotide is hybridized to the nucleic acid. The extension oligonucleotide then is extended by one or more nucleotides, often 1, 2, or 3 nucleotides, and the number and/or type of nucleotides that are added to the extension oligonucleotide determine which polymorphic variant or variants are present. Oligonucleotide extension methods are disclosed, for example, in U.S. Pat. Nos. 4,656,127; 4,851,331; 5,679,524; 5,834,189; 5,876,934; 5,908,755; 5,912,118; 5,976,802; 5,981,186; 6,004,744; 6,013,431; 6,017,702; 6,046,005; 6,087,095; 6,210,891; and WO 01/20039. The extension products can be detected in any manner, such as by fluorescence methods (see, e.g., Chen & Kwok, Nucleic Acids Research 25: 347-353 (1997) and Chen et al., Proc. Natl. Acad. Sci. USA 94/20: 10756-10761 (1997)) or by mass spectrometric methods (e.g., MALDI-TOF mass spectrometry) and other methods described herein. Oligonucleotide extension methods using mass spectrometry are described, for example, in U.S. Pat. Nos. 5,547,835; 5,605,798; 5,691,141; 5,849,542; 5,869,242; 5,928,906; 6,043,031; 6,194,144; and 6,258,538.

Microsequencing detection methods often incorporate an amplification process that proceeds the extension step. The amplification process typically amplifies a region from a nucleic acid sample that comprises the polymorphic site. Amplification can be carried out using methods described above, or for example using a pair of oligonucleotide primers in a polymerase chain reaction (PCR), in which one oligonucleotide primer typically is complementary to a region 3′ of the polymorphism and the other typically is complementary to a region 5′ of the polymorphism. A PCR primer pair may be used in methods disclosed in U.S. Pat. Nos. 4,683,195; 4,683,202, 4,965,188; 5,656,493; 5,998,143; 6,140,054; WO 01/27327; and WO 01/27329 for example. PCR primer pairs may also be used in any commercially available machines that perform PCR, such as any of the GeneAmp™ Systems available from Applied Biosystems.

Other appropriate sequencing methods include multiplex polony sequencing (as described in Shendure et al., Accurate Multiplex Polony Sequencing of an Evolved Bacterial Genome, Sciencexpress, Aug. 4, 2005, pg 1 available at www.sciencexpress.org/4 Aug. 2005/Pagel/10.1126/science.1117389, incorporated herein by reference), which employs immobilized microbeads, and sequencing in microfabricated picoliter reactors (as described in Margulies et al., Genome Sequencing in Microfabricated High-Density Picolitre Reactors, Nature, August 2005, available at www.nature.cominature (published online 31 Jul. 2005, doi:10.1038/nature03959, incorporated herein by reference).

Whole genome sequencing may also be used for discriminating alleles of RNA transcripts, in some embodiments. Examples of whole genome sequencing methods include, but are not limited to, nanopore-based sequencing methods, sequencing by synthesis and sequencing by ligation, as described above.

Nucleic acid variants can also be detected using standard electrophoretic techniques. Although the detection step can sometimes be preceded by an amplification step, amplification is not required in the embodiments described herein. Examples of methods for detection and quantification of a nucleic acid using electrophoretic techniques can be found in the art. A non-limiting example comprises running a sample (e.g., mixed nucleic acid sample isolated from maternal serum, or amplification nucleic acid species, for example) in an agarose or polyacrylamide gel. The gel may be labeled (e.g., stained) with ethidium bromide (see, Sambrook and Russell, Molecular Cloning: A Laboratory Manual 3d ed., 2001). The presence of a band of the same size as the standard control is an indication of the presence of a target nucleic acid sequence, the amount of which may then be compared to the control based on the intensity of the band, thus detecting and quantifying the target sequence of interest. In some embodiments, restriction enzymes capable of distinguishing between maternal and paternal alleles may be used to detect and quantify target nucleic acid species. In certain embodiments, oligonucleotide probes specific to a sequence of interest are used to detect the presence of the target sequence of interest. The oligonucleotides can also be used to indicate the amount of the target nucleic acid molecules in comparison to the standard control, based on the intensity of signal imparted by the probe.

Sequence-specific probe hybridization can be used to detect a particular nucleic acid in a mixture or mixed population comprising other species of nucleic acids. Under sufficiently stringent hybridization conditions, the probes hybridize specifically only to substantially complementary sequences. The stringency of the hybridization conditions can be relaxed to tolerate varying amounts of sequence mismatch. A number of hybridization formats are known in the art, which include but are not limited to, solution phase, solid phase, or mixed phase hybridization assays. The following articles provide an overview of the various hybridization assay formats: Singer et al., Biotechniques 4:230, 1986; Haase et al., Methods in Virology, pp. 189-226, 1984; Wilkinson, In situ Hybridization, Wilkinson ed., IRL Press, Oxford University Press, Oxford; and Hames and Higgins eds., Nucleic Acid Hybridization: A Practical Approach, IRL Press, 1987.

Hybridization complexes can be detected by techniques known in the art. Nucleic acid probes capable of specifically hybridizing to a target nucleic acid (e.g., mRNA or DNA) can be labeled by any suitable method, and the labeled probe used to detect the presence of hybridized nucleic acids. One commonly used method of detection is autoradiography, using probes labeled with ³H, ¹²⁵I, ³⁵S, ¹⁴C, ³²P, ³³P or the like. The choice of radioactive isotope depends on research preferences due to ease of synthesis, stability, and half-lives of the selected isotopes. Other labels include compounds (e.g., biotin and digoxigenin), which bind to antiligands or antibodies labeled with fluorophores, chemiluminescent agents, and enzymes. In some embodiments, probes can be conjugated directly with labels such as fluorophores, chemiluminescent agents or enzymes. The choice of label depends on sensitivity required, ease of conjugation with the probe, stability requirements, and available instrumentation.

In embodiments, fragment analysis (referred to herein as “FA”) methods are used for molecular profiling. Fragment analysis (FA) includes techniques such as restriction fragment length polymorphism (RFLP) and/or (amplified fragment length polymorphism). If a nucleotide variant in the target DNA corresponding to the one or more genes results in the elimination or creation of a restriction enzyme recognition site, then digestion of the target DNA with that particular restriction enzyme will generate an altered restriction fragment length pattern. Thus, a detected RFLP or AFLP will indicate the presence of a particular nucleotide variant.

Terminal restriction fragment length polymorphism (TRFLP) works by PCR amplification of DNA using primer pairs that have been labeled with fluorescent tags. The PCR products are digested using RFLP enzymes and the resulting patterns are visualized using a DNA sequencer. The results are analyzed either by counting and comparing bands or peaks in the TRFLP profile, or by comparing bands from one or more TRFLP runs in a database.

The sequence changes directly involved with an RFLP can also be analyzed more quickly by PCR. Amplification can be directed across the altered restriction site, and the products digested with the restriction enzyme. This method has been called Cleaved Amplified Polymorphic Sequence (CAPS). Alternatively, the amplified segment can be analyzed by Allele specific oligonucleotide (ASO) probes, a process that is sometimes assessed using a Dot blot.

A variation on AFLP is cDNA-AFLP, which can be used to quantify differences in gene expression levels.

Another useful approach is the single-stranded conformation polymorphism assay (SSCA), which is based on the altered mobility of a single-stranded target DNA spanning the nucleotide variant of interest. A single nucleotide change in the target sequence can result in different intramolecular base pairing pattern, and thus different secondary structure of the single-stranded DNA, which can be detected in a non-denaturing gel. See Orita et al., Proc. Natl. Acad. Sci. USA, 86:2776-2770 (1989). Denaturing gel-based techniques such as clamped denaturing gel electrophoresis (CDGE) and denaturing gradient gel electrophoresis (DGGE) detect differences in migration rates of mutant sequences as compared to wild-type sequences in denaturing gel. See Miller et al., Biotechniques, 5:1016-24 (1999); Sheffield et al., Am. J. Hum, Genet., 49:699-706 (1991); Wartell et al., Nucleic Acids Res., 18:2699-2705 (1990); and Sheffield et al., Proc. Natl. Acad. Sci. USA, 86:232-236 (1989). In addition, the double-strand conformation analysis (DSCA) can also be useful in the present invention. See Arguello et al., Nat. Genet., 18:192-194 (1998).

The presence or absence of a nucleotide variant at a particular locus in the one or more genes of an individual can also be detected using the amplification refractory mutation system (ARMS) technique. See e.g., European Patent No. 0,332,435; Newton et al., Nucleic Acids Res., 17:2503-2515 (1989); Fox et al., Br. J. Cancer, 77:1267-1274 (1998); Robertson et al., Eur. Respir. J., 12:477-482 (1998). In the ARMS method, a primer is synthesized matching the nucleotide sequence immediately 5′ upstream from the locus being tested except that the 3′-end nucleotide which corresponds to the nucleotide at the locus is a predetermined nucleotide. For example, the 3′-end nucleotide can be the same as that in the mutated locus. The primer can be of any suitable length so long as it hybridizes to the target DNA under stringent conditions only when its 3′-end nucleotide matches the nucleotide at the locus being tested. Preferably the primer has at least 12 nucleotides, more preferably from about 18 to 50 nucleotides. If the individual tested has a mutation at the locus and the nucleotide therein matches the 3′-end nucleotide of the primer, then the primer can be further extended upon hybridizing to the target DNA template, and the primer can initiate a PCR amplification reaction in conjunction with another suitable PCR primer. In contrast, if the nucleotide at the locus is of wild type, then primer extension cannot be achieved. Various forms of ARMS techniques developed in the past few years can be used. See e.g., Gibson et al., Clin. Chem. 43:1336-1341 (1997).

Similar to the ARMS technique is the mini sequencing or single nucleotide primer extension method, which is based on the incorporation of a single nucleotide. An oligonucleotide primer matching the nucleotide sequence immediately 5′ to the locus being tested is hybridized to the target DNA, mRNA or miRNA in the presence of labeled dideoxyribonucleotides. A labeled nucleotide is incorporated or linked to the primer only when the dideoxyribonucleotides matches the nucleotide at the variant locus being detected. Thus, the identity of the nucleotide at the variant locus can be revealed based on the detection label attached to the incorporated dideoxyribonucleotides. See Syvanen et al., Genomics, 8:684-692 (1990); Shumaker et al., Hum. Mutat., 7:346-354 (1996); Chen et al., Genome Res., 10:549-547 (2000).

Another set of techniques useful in the present invention is the so-called “oligonucleotide ligation assay” (OLA) in which differentiation between a wild-type locus and a mutation is based on the ability of two oligonucleotides to anneal adjacent to each other on the target DNA molecule allowing the two oligonucleotides joined together by a DNA ligase. See Landergren et al., Science, 241:1077-1080 (1988); Chen et al, Genome Res., 8:549-556 (1998); Iannone et al., Cytometry, 39:131-140 (2000). Thus, for example, to detect a single-nucleotide mutation at a particular locus in the one or more genes, two oligonucleotides can be synthesized, one having the sequence just 5′ upstream from the locus with its 3′ end nucleotide being identical to the nucleotide in the variant locus of the particular gene, the other having a nucleotide sequence matching the sequence immediately 3′ downstream from the locus in the gene. The oligonucleotides can be labeled for the purpose of detection. Upon hybridizing to the target gene under a stringent condition, the two oligonucleotides are subject to ligation in the presence of a suitable ligase. The ligation of the two oligonucleotides would indicate that the target DNA has a nucleotide variant at the locus being detected.

Detection of small genetic variations can also be accomplished by a variety of hybridization-based approaches. Allele-specific oligonucleotides are most useful. See Conner et al., Proc. Natl. Acad. Sci. USA, 80:278-282 (1983); Saiki et al, Proc. Natl. Acad. Sci. USA, 86:6230-6234 (1989). Oligonucleotide probes (allele-specific) hybridizing specifically to a gene allele having a particular gene variant at a particular locus but not to other alleles can be designed by methods known in the art. The probes can have a length of, e.g., from 10 to about 50 nucleotide bases. The target DNA and the oligonucleotide probe can be contacted with each other under conditions sufficiently stringent such that the nucleotide variant can be distinguished from the wild-type gene based on the presence or absence of hybridization. The probe can be labeled to provide detection signals. Alternatively, the allele-specific oligonucleotide probe can be used as a PCR amplification primer in an “allele-specific PCR” and the presence or absence of a PCR product of the expected length would indicate the presence or absence of a particular nucleotide variant.

Other useful hybridization-based techniques allow two single-stranded nucleic acids annealed together even in the presence of mismatch due to nucleotide substitution, insertion or deletion. The mismatch can then be detected using various techniques. For example, the annealed duplexes can be subject to electrophoresis. The mismatched duplexes can be detected based on their electrophoretic mobility that is different from the perfectly matched duplexes. See Cariello, Human Genetics, 42:726 (1988). Alternatively, in an RNase protection assay, a RNA probe can be prepared spanning the nucleotide variant site to be detected and having a detection marker. See Giunta et al., Diagn. Mol. Path., 5:265-270 (1996); Finkelstein et al., Genomics, 7:167-172 (1990); Kinszler et al., Science 251:1366-1370 (1991). The RNA probe can be hybridized to the target DNA or mRNA forming a heteroduplex that is then subject to the ribonuclease RNase A digestion. RNase A digests the RNA probe in the heteroduplex only at the site of mismatch. The digestion can be determined on a denaturing electrophoresis gel based on size variations. In addition, mismatches can also be detected by chemical cleavage methods known in the art. See e.g., Roberts et al., Nucleic Acids Res., 25:3377-3378 (1997).

In the mutS assay, a probe can be prepared matching the gene sequence surrounding the locus at which the presence or absence of a mutation is to be detected, except that a predetermined nucleotide is used at the variant locus. Upon annealing the probe to the target DNA to form a duplex, the E. coli mutS protein is contacted with the duplex. Since the mutS protein binds only to heteroduplex sequences containing a nucleotide mismatch, the binding of the mutS protein will be indicative of the presence of a mutation. See Modrich et al., Ann. Rev. Genet., 25:229-253 (1991).

A great variety of improvements and variations have been developed in the art on the basis of the above-described basic techniques which can be useful in detecting mutations or nucleotide variants in the present invention. For example, the “sunrise probes” or “molecular beacons” use the fluorescence resonance energy transfer (FRET) property and give rise to high sensitivity. See Wolf et al., Proc. Nat. Acad. Sci. USA, 85:8790-8794 (1988). Typically, a probe spanning the nucleotide locus to be detected are designed into a hairpin-shaped structure and labeled with a quenching fluorophore at one end and a reporter fluorophore at the other end. In its natural state, the fluorescence from the reporter fluorophore is quenched by the quenching fluorophore due to the proximity of one fluorophore to the other. Upon hybridization of the probe to the target DNA, the 5′ end is separated apart from the 3′-end and thus fluorescence signal is regenerated. See Nazarenko et al., Nucleic Acids Res., 25:2516-2521 (1997); Rychlik et al., Nucleic Acids Res., 17:8543-8551 (1989); Sharkey et al., Bio/Technology 12:506-509 (1994); Tyagi et al., Nat. Biotechnol., 14:303-308 (1996); Tyagi et al., Nat. Biotechnol., 16:49-53 (1998). The homo-tag assisted non-dimer system (HANDS) can be used in combination with the molecular beacon methods to suppress primer-dimer accumulation. See Brownie et al., Nucleic Acids Res., 25:3235-3241 (1997).

Dye-labeled oligonucleotide ligation assay is a FRET-based method, which combines the OLA assay and PCR. See Chen et al., Genome Res. 8:549-556 (1998). TaqMan is another FRET-based method for detecting nucleotide variants. A TaqMan probe can be oligonucleotides designed to have the nucleotide sequence of the gene spanning the variant locus of interest and to differentially hybridize with different alleles. The two ends of the probe are labeled with a quenching fluorophore and a reporter fluorophore, respectively. The TaqMan probe is incorporated into a PCR reaction for the amplification of a target gene region containing the locus of interest using Taq polymerase. As Taq polymerase exhibits 5′-3′ exonuclease activity but has no 3′-5′ exonuclease activity, if the TaqMan probe is annealed to the target DNA template, the 5′-end of the TaqMan probe will be degraded by Taq polymerase during the PCR reaction thus separating the reporting fluorophore from the quenching fluorophore and releasing fluorescence signals. See Holland et al., Proc. Natl. Acad. Sci. USA, 88:7276-7280 (1991); Kalinina et al., Nucleic Acids Res., 25:1999-2004 (1997); Whitcombe et al., Clin. Chem., 44:918-923 (1998).

In addition, the detection in the present invention can also employ a chemiluminescence-based technique. For example, an oligonucleotide probe can be designed to hybridize to either the wild-type or a variant gene locus but not both. The probe is labeled with a highly chemiluminescent acridinium ester. Hydrolysis of the acridinium ester destroys chemiluminescence. The hybridization of the probe to the target DNA prevents the hydrolysis of the acridinium ester. Therefore, the presence or absence of a particular mutation in the target DNA is determined by measuring chemiluminescence changes. See Nelson et al., Nucleic Acids Res., 24:4998-5003 (1996).

The detection of genetic variation in the gene in accordance with the present invention can also be based on the “base excision sequence scanning” (BESS) technique. The BESS method is a PCR-based mutation scanning method. BESS T-Scan and BESS G-Tracker are generated which are analogous to T and G ladders of dideoxy sequencing. Mutations are detected by comparing the sequence of normal and mutant DNA. See, e.g., Hawkins et al., Electrophoresis, 20:1171-1176 (1999).

Mass spectrometry can be used for molecular profiling according to the invention. See Graber et al., Curr. Opin. Biotechnol., 9:14-18 (1998). For example, in the primer oligo base extension (PROBE™) method, a target nucleic acid is immobilized to a solid-phase support. A primer is annealed to the target immediately 5′ upstream from the locus to be analyzed. Primer extension is carried out in the presence of a selected mixture of deoxyribonucleotides and dideoxyribonucleotides. The resulting mixture of newly extended primers is then analyzed by MALDI-TOF. See e.g., Monforte et al., Nat. Med., 3:360-362 (1997).

In addition, the microchip or microarray technologies are also applicable to the detection method of the present invention. Essentially, in microchips, a large number of different oligonucleotide probes are immobilized in an array on a substrate or carrier, e.g., a silicon chip or glass slide. Target nucleic acid sequences to be analyzed can be contacted with the immobilized oligonucleotide probes on the microchip. See Lipshutz et al., Biotechniques, 19:442-447 (1995); Chee et al., Science, 274:610-614 (1996); Kozal et al., Nat. Med. 2:753-759 (1996); Hacia et al., Nat. Genet., 14:441-447 (1996); Saiki et al., Proc. Natl. Acad. Sci. USA, 86:6230-6234 (1989); Gingeras et al., Genome Res., 8:435-448 (1998). Alternatively, the multiple target nucleic acid sequences to be studied are fixed onto a substrate and an array of probes is contacted with the immobilized target sequences. See Drmanac et al., Nat. Biotechnol., 16:54-58 (1998). Numerous microchip technologies have been developed incorporating one or more of the above described techniques for detecting mutations. The microchip technologies combined with computerized analysis tools allow fast screening in a large scale. The adaptation of the microchip technologies to the present invention will be apparent to a person of skill in the art apprised of the present disclosure. See, e.g., U.S. Pat. No. 5,925,525 to Fodor et al; Wilgenbus et al., J. Mol. Med., 77:761-786 (1999); Graber et al., Curr. Opin. Biotechnol., 9:14-18 (1998); Hacia et al., Nat. Genet., 14:441-447 (1996); Shoemaker et al., Nat. Genet., 14:450-456 (1996); DeRisi et al., Nat. Genet., 14:457-460 (1996); Chee et al., Nat. Genet., 14:610-614 (1996); Lockhart et al., Nat. Genet., 14:675-680 (1996); Drobyshev et al., Gene, 188:45-52 (1997).

As is apparent from the above survey of the suitable detection techniques, it may or may not be necessary to amplify the target DNA, i.e., the gene, cDNA, mRNA, miRNA, or a portion thereof to increase the number of target DNA molecule, depending on the detection techniques used. For example, most PCR-based techniques combine the amplification of a portion of the target and the detection of the mutations. PCR amplification is well known in the art and is disclosed in U.S. Pat. Nos. 4,683,195 and 4,800,159, both which are incorporated herein by reference. For non-PCR-based detection techniques, if necessary, the amplification can be achieved by, e.g., in vivo plasmid multiplication, or by purifying the target DNA from a large amount of tissue or cell samples. See generally, Sambrook et al., Molecular Cloning: A Laboratory Manual, 2^{nd} ed., Cold Spring Harbor Laboratory, Cold Spring Harbor, N.Y., 1989. However, even with scarce samples, many sensitive techniques have been developed in which small genetic variations such as single-nucleotide substitutions can be detected without having to amplify the target DNA in the sample. For example, techniques have been developed that amplify the signal as opposed to the target DNA by, e.g., employing branched DNA or dendrimers that can hybridize to the target DNA. The branched or dendrimer DNAs provide multiple hybridization sites for hybridization probes to attach thereto thus amplifying the detection signals. See Detmer et al., J. Clin. Microbiol., 34:901-907 (1996); Collins et al., Nucleic Acids Res., 25:2979-2984 (1997); Horn et al., Nucleic Acids Res., 25:4835-4841 (1997); Horn et al., Nucleic Acids Res., 25:4842-4849 (1997); Nilsen et al., J. Theor. Biol., 187:273-284 (1997).

The Invader™ assay is another technique for detecting single nucleotide variations that can be used for molecular profiling according to the invention. The Invader™ assay uses a novel linear signal amplification technology that improves upon the long turnaround times required of the typical PCR DNA sequenced-based analysis. See Cooksey et al., Antimicrobial Agents and Chemotherapy 44:1296-1301 (2000). This assay is based on cleavage of a unique secondary structure formed between two overlapping oligonucleotides that hybridize to the target sequence of interest to form a “flap.” Each “flap” then generates thousands of signals per hour. Thus, the results of this technique can be easily read, and the methods do not require exponential amplification of the DNA target. The Invader™ system uses two short DNA probes, which are hybridized to a DNA target. The structure formed by the hybridization event is recognized by a special cleavase enzyme that cuts one of the probes to release a short DNA “flap.” Each released “flap” then binds to a fluorescently-labeled probe to form another cleavage structure. When the cleavase enzyme cuts the labeled probe, the probe emits a detectable fluorescence signal. See e.g. Lyamichev et al., Nat. Biotechnol., 17:292-296 (1999).

The rolling circle method is another method that avoids exponential amplification. Lizardi et al., Nature Genetics, 19:225-232 (1998) (which is incorporated herein by reference). For example, Sniper™ a commercial embodiment of this method, is a sensitive, high-throughput SNP scoring system designed for the accurate fluorescent detection of specific variants. For each nucleotide variant, two linear, allele-specific probes are designed. The two allele-specific probes are identical with the exception of the 3′-base, which is varied to complement the variant site. In the first stage of the assay, target DNA is denatured and then hybridized with a pair of single, allele-specific, open-circle oligonucleotide probes. When the 3′-base exactly complements the target DNA, ligation of the probe will preferentially occur. Subsequent detection of the circularized oligonucleotide probes is by rolling circle amplification, whereupon the amplified probe products are detected by fluorescence. See Clark and Pickering, Life Science News 6, 2000, Amersham Pharmacia Biotech (2000).

A number of other techniques that avoid amplification all together include, e.g., surface-enhanced resonance Raman scattering (SERRS), fluorescence correlation spectroscopy, and single-molecule electrophoresis. In SERRS, a chromophore-nucleic acid conjugate is absorbed onto colloidal silver and is irradiated with laser light at a resonant frequency of the chromophore. See Graham et al., Anal. Chem., 69:4703-4707 (1997). The fluorescence correlation spectroscopy is based on the spatio-temporal correlations among fluctuating light signals and trapping single molecules in an electric field. See Eigen et al., Proc. Natl. Acad. Sci. USA, 91:5740-5747 (1994). In single-molecule electrophoresis, the electrophoretic velocity of a fluorescently tagged nucleic acid is determined by measuring the time required for the molecule to travel a predetermined distance between two laser beams. See Castro et al., Anal. Chem., 67:3181-3186 (1995).

In addition, the allele-specific oligonucleotides (ASO) can also be used in in situ hybridization using tissues or cells as samples. The oligonucleotide probes which can hybridize differentially with the wild-type gene sequence or the gene sequence harboring a mutation may be labeled with radioactive isotopes, fluorescence, or other detectable markers. In situ hybridization techniques are well known in the art and their adaptation to the present invention for detecting the presence or absence of a nucleotide variant in the one or more gene of a particular individual should be apparent to a skilled artisan apprised of this disclosure.

Accordingly, the presence or absence of one or more genes nucleotide variant or amino acid variant in an individual can be determined using any of the detection methods described above.

Typically, once the presence or absence of one or more gene nucleotide variants or amino acid variants is determined, physicians or genetic counselors or patients or other researchers may be informed of the result. Specifically the result can be cast in a transmittable form that can be communicated or transmitted to other researchers or physicians or genetic counselors or patients. Such a form can vary and can be tangible or intangible. The result with regard to the presence or absence of a nucleotide variant of the present invention in the individual tested can be embodied in descriptive statements, diagrams, photographs, charts, images or any other visual forms. For example, images of gel electrophoresis of PCR products can be used in explaining the results. Diagrams showing where a variant occurs in an individual's gene are also useful in indicating the testing results. The statements and visual forms can be recorded on a tangible media such as papers, computer readable media such as floppy disks, compact disks, etc., or on an intangible media, e.g., an electronic media in the form of email or website on internet or intranet. In addition, the result with regard to the presence or absence of a nucleotide variant or amino acid variant in the individual tested can also be recorded in a sound form and transmitted through any suitable media, e.g., analog or digital cable lines, fiber optic cables, etc., via telephone, facsimile, wireless mobile phone, internet phone and the like.

Thus, the information and data on a test result can be produced anywhere in the world and transmitted to a different location. For example, when a genotyping assay is conducted offshore, the information and data on a test result may be generated and cast in a transmittable form as described above. The test result in a transmittable form thus can be imported into the U.S. Accordingly, the present invention also encompasses a method for producing a transmittable form of information on the genotype of the two or more suspected cancer samples from an individual. The method comprises the steps of (1) determining the genotype of the DNA from the samples according to methods of the present invention; and (2) embodying the result of the determining step in a transmittable form. The transmittable form is the product of the production method.

### In Situ Hybridization

In situ hybridization assays are well known and are generally described in Angerer et al., Methods Enzymol. 152:649-660 (1987). In an in situ hybridization assay, cells, e.g., from a biopsy, are fixed to a solid support, typically a glass slide. If DNA is to be probed, the cells are denatured with heat or alkali. The cells are then contacted with a hybridization solution at a moderate temperature to permit annealing of specific probes that are labeled. The probes are preferably labeled, e.g., with radioisotopes or fluorescent reporters, or enzymatically. FISH (fluorescence in situ hybridization) uses fluorescent probes that bind to only those parts of a sequence with which they show a high degree of sequence similarity. CISH (chromogenic in situ hybridization) uses conventional peroxidase or alkaline phosphatase reactions visualized under a standard bright-field microscope.

In situ hybridization can be used to detect specific gene sequences in tissue sections or cell preparations by hybridizing the complementary strand of a nucleotide probe to the sequence of interest. Fluorescent in situ hybridization (FISH) uses a fluorescent probe to increase the sensitivity of in situ hybridization.

FISH is a cytogenetic technique used to detect and localize specific polynucleotide sequences in cells. For example, FISH can be used to detect DNA sequences on chromosomes. FISH can also be used to detect and localize specific RNAs, e.g., mRNAs, within tissue samples. In FISH uses fluorescent probes that bind to specific nucleotide sequences to which they show a high degree of sequence similarity. Fluorescence microscopy can be used to find out whether and where the fluorescent probes are bound. In addition to detecting specific nucleotide sequences, e.g., translocations, fusion, breaks, duplications and other chromosomal abnormalities, FISH can help define the spatial-temporal patterns of specific gene copy number and/or gene expression within cells and tissues.

Various types of FISH probes can be used to detect chromosome translocations. Dual color, single fusion probes can be useful in detecting cells possessing a specific chromosomal translocation. The DNA probe hybridization targets are located on one side of each of the two genetic breakpoints. “Extra signal” probes can reduce the frequency of normal cells exhibiting an abnormal FISH pattern due to the random co-localization of probe signals in a normal nucleus. One large probe spans one breakpoint, while the other probe flanks the breakpoint on the other gene. Dual color, break apart probes are useful in cases where there may be multiple translocation partners associated with a known genetic breakpoint. This labeling scheme features two differently colored probes that hybridize to targets on opposite sides of a breakpoint in one gene. Dual color, dual fusion probes can reduce the number of normal nuclei exhibiting abnormal signal patterns. The probe offers advantages in detecting low levels of nuclei possessing a simple balanced translocation. Large probes span two breakpoints on different chromosomes. Such probes are available as Vysis probes from Abbott Laboratories, Abbott Park, Ill.

CISH, or chromogenic in situ hybridization, is a process in which a labeled complementary DNA or RNA strand is used to localize a specific DNA or RNA sequence in a tissue specimen. CISH methodology can be used to evaluate gene amplification, gene deletion, chromosome translocation, and chromosome number. CISH can use conventional enzymatic detection methodology, e.g., horseradish peroxidase or alkaline phosphatase reactions, visualized under a standard bright-field microscope. In a common embodiment, a probe that recognizes the sequence of interest is contacted with a sample. An antibody or other binding agent that recognizes the probe, e.g., via a label carried by the probe, can be used to target an enzymatic detection system to the site of the probe. In some systems, the antibody can recognize the label of a FISH probe, thereby allowing a sample to be analyzed using both FISH and CISH detection. CISH can be used to evaluate nucleic acids in multiple settings, e.g., formalin-fixed, paraffin-embedded (FFPE) tissue, blood or bone marrow smear, metaphase chromosome spread, and/or fixed cells. In an embodiment, CISH is performed following the methodology in the SPoT-Light® HER2 CISH Kit available from Life Technologies (Carlsbad, Calif.) or similar CISH products available from Life Technologies. The SPoT-Light® HER2 CISH Kit itself is FDA approved for in vitro diagnostics and can be used for molecular profiling of HER2. CISH can be used in similar applications as FISH. Thus, one of skill will appreciate that reference to molecular profiling using FISH herein can be performed using CISH, unless otherwise specified.

Silver-enhanced in situ hybridization (SISH) is similar to CISH, but with SISH the signal appears as a black coloration due to silver precipitation instead of the chromogen precipitates of CISH.

Modifications of the in situ hybridization techniques can be used for molecular profiling according to the invention. Such modifications comprise simultaneous detection of multiple targets, e.g., Dual ISH, Dual color CISH, bright field double in situ hybridization (BDISH). See e.g., the FDA approved INFORM HER2 Dual ISH DNA Probe Cocktail kit from Ventana Medical Systems, Inc. (Tucson, Ariz.); DuoCISH™, a dual color CISH kit developed by Dako Denmark A/S (Denmark).

Comparative Genomic Hybridization (CGH) comprises a molecular cytogenetic method of screening tumor samples for genetic changes showing characteristic patterns for copy number changes at chromosomal and subchromosomal levels. Alterations in patterns can be classified as DNA gains and losses. CGH employs the kinetics of in situ hybridization to compare the copy numbers of different DNA or RNA sequences from a sample, or the copy numbers of different DNA or RNA sequences in one sample to the copy numbers of the substantially identical sequences in another sample. In many useful applications of CGH, the DNA or RNA is isolated from a subject cell or cell population. The comparisons can be qualitative or quantitative. Procedures are described that permit determination of the absolute copy numbers of DNA sequences throughout the genome of a cell or cell population if the absolute copy number is known or determined for one or several sequences. The different sequences are discriminated from each other by the different locations of their binding sites when hybridized to a reference genome, usually metaphase chromosomes but in certain cases interphase nuclei. The copy number information originates from comparisons of the intensities of the hybridization signals among the different locations on the reference genome. The methods, techniques and applications of CGH are known, such as described in U.S. Pat. No. 6,335,167, and in U.S. App. Ser. No. 60/804,818, the relevant parts of which are herein incorporated by reference.

In an embodiment, CGH used to compare nucleic acids between diseased and healthy tissues. The method comprises isolating DNA from disease tissues (e.g., tumors) and reference tissues (e.g., healthy tissue) and labeling each with a different “color” or fluor. The two samples are mixed and hybridized to normal metaphase chromosomes. In the case of array or matrix CGH, the hybridization mixing is done on a slide with thousands of DNA probes. A variety of detection system can be used that basically determine the color ratio along the chromosomes to determine DNA regions that might be gained or lost in the diseased samples as compared to the reference.

### Molecular Profiling for Treatment Selection

The methods of the invention provide a candidate treatment selection for a subject in need thereof. Molecular profiling can be used to identify one or more candidate therapeutic agents for an individual suffering from a condition in which one or more of the biomarkers disclosed herein are targets for treatment. For example, the method can identify one or more chemotherapy treatments for a cancer. In an aspect, the invention provides a method comprising: performing an immunohistochemistry (IHC) analysis on a sample from the subject to determine an IHC expression profile on at least five proteins; performing a microarray analysis on the sample to determine a microarray expression profile on at least ten genes; performing a fluorescent in-situ hybridization (FISH) analysis on the sample to determine a FISH mutation profile on at least one gene; performing DNA sequencing on the sample to determine a sequencing mutation profile on at least one gene; and comparing the IHC expression profile, microarray expression profile, FISH mutation profile and sequencing mutation profile against a rules database, wherein the rules database comprises a mapping of treatments whose biological activity is known against diseased cells that: i) overexpress or underexpress one or more proteins included in the IHC expression profile; ii) overexpress or underexpress one or more genes included in the microarray expression profile; iii) have zero or more mutations in one or more genes included in the FISH mutation profile; and/or iv) have zero or more mutations in one or more genes included in the sequencing mutation profile; and identifying the treatment if the comparison against the rules database indicates that the treatment should have biological activity against the diseased cells; and the comparison against the rules database does not contraindicate the treatment for treating the diseased cells. The disease can be a cancer. The molecular profiling steps can be performed in any order. In some embodiments, not all of the molecular profiling steps are performed. As a non-limiting example, microarray analysis is not performed if the sample quality does not meet a threshold value, as described herein. In another example, sequencing is performed only if FISH analysis meets a threshold value. Any relevant biomarker can be assessed using one or more of the molecular profiling techniques described herein or known in the art. The marker need only have some direct or indirect association with a treatment to be useful.

Molecular profiling comprises the profiling of at least one gene (or gene product) for each assay technique that is performed. Different numbers of genes can be assayed with different techniques. Any marker disclosed herein that is associated directly or indirectly with a target therapeutic can be assessed. For example, any “druggable target” comprising a target that can be modulated with a therapeutic agent such as a small molecule or binding agent such as an antibody, is a candidate for inclusion in the molecular profiling methods of the invention. The target can also be indirectly drug associated, such as a component of a biological pathway that is affected by the associated drug. The molecular profiling can be based on either the gene, e.g., DNA sequence, and/or gene product, e.g., mRNA or protein. Such nucleic acid and/or polypeptide can be profiled as applicable as to presence or absence, level or amount, activity, mutation, sequence, haplotype, rearrangement, copy number, or other measurable characteristic. In some embodiments, a single gene and/or one or more corresponding gene products is assayed by more than one molecular profiling technique. A gene or gene product (also referred to herein as “marker” or “biomarker”), e.g., an mRNA or protein, is assessed using applicable techniques (e.g., to assess DNA, RNA, protein), including without limitation FISH, microarray, IHC, sequencing or immunoassay. Therefore, any of the markers disclosed herein can be assayed by a single molecular profiling technique or by multiple methods disclosed herein (e.g., a single marker is profiled by one or more of IHC, FISH, sequencing, microarray, etc.). In some embodiments, at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95 or at least about 100 genes or gene products are profiled by at least one technique, a plurality of techniques, or using a combination of FISH, microarray, IHC, and sequencing. In some embodiments, at least about 100, 200, 300, 400, 500, 600, 700, 800, 900, 1000, 2000, 3000, 4000, 5000, 6000, 7000, 8000, 9000, 10,000, 11,000, 12,000, 13,000, 14,000, 15,000, 16,000, 17,000, 18,000, 19,000, 20,000, 21,000, 22,000, 23,000, 24,000, 25,000, 26,000, 27,000, 28,000, 29,000, 30,000, 31,000, 32,000, 33,000, 34,000, 35,000, 36,000, 37,000, 38,000, 39,000, 40,000, 41,000, 42,000, 43,000, 44,000, 45,000, 46,000, 47,000, 48,000, 49,000, or at least 50,000 genes or gene products are profiled using various techniques. The number of markers assayed can depend on the technique used. For example, microarray and massively parallel sequencing lend themselves to high throughput analysis. Because molecular profiling queries molecular characteristics of the tumor itself, this approach provides information on therapies that might not otherwise be considered based on the lineage of the tumor.

In some embodiments, a sample from a subject in need thereof is profiled using methods which include but are not limited to IHC expression profiling, microarray expression profiling, FISH mutation profiling, and/or sequencing mutation profiling (such as by PCR, RT-PCR, pyrosequencing) for one or more of the following: ABCC1, ABCG2, ACE2, ADA, ADH1C, ADH4, AGT, AR, AREG, ASNS, BCL2, BCRP, BDCA1, beta III tubulin, BIRC5, B-RAF, BRCA1, BRCA2, CA2, caveolin, CD20, CD25, CD33, CD52, CDA, CDKN2A, CDKN1A, CDKN1B, CDK2, CDW52, CES2, CK 14, CK 17, CK 5/6, c-KIT, c-Met, c-Myc, COX-2, Cyclin D1, DCK, DHFR, DNMT1, DNMT3A, DNMT3B, E-Cadherin, ECGF1, EGFR, EML4-ALK fusion, EPHA2, Epiregulin, ER, ERBR2, ERCC1, ERCC3, EREG, ESR1, FLT1, folate receptor, FOLR1, FOLR2, FSHB, FSHPRH1, FSHR, FYN, CART, GNA11, GNAQ, GNRH1, GNRHR1, GSTP1, HCK, HDAC1, hENT-1, Her2/Neu, HGF, HIF1A, HIG1, HSP90, HSP90AA1, HSPCA, IGF-1R, IGFRBP, IGFRBP3, IGFRBP4, IGFRBP5, IL13RA1, IL2RA, KDR, Ki67, KIT, K-RAS, LCK, LTB, Lymphotoxin Beta Receptor, LYN, MET, MGMT, MLH1, MMR, MRP1, MS4A1, MSH2, MSH5, Myc, NFKB1, NFKB2, NFKBIA, NRAS, ODC1, OGFR, p16, p21, p27, p53, p95, PARP-1, PDGFC, PDGFR, PDGFRA, PDGFRB, PGP, PGR, PI3K, POLA, POLA1, PPARG, PPARGC1, PR, PTEN, PTGS2, PTPN12, RAF1, RARA, ROS1, RRM1, RRM2, RRM2B, RXRB, RXRG, SIK2, SPARC, SRC, SSTR1, SSTR2, SSTR3, SSTR4, SSTR5, Survivin, TK1, TLE3, TNF, TOP1, TOP2A, TOP2B, TS, TUBB3, TXN, TXNRD1, TYMS, VDR, VEGF, VEGFA, VEGFC, VHL, YES1, ZAP70.

Table 2 provides a listing of gene and corresponding protein symbols and names of many of the molecular profiling targets that are analyzed according to the methods of the invention. As understood by those of skill in the art, genes and proteins have developed a number of alternative names in the scientific literature. Thus, the listing in Table 2 comprises an illustrative but not exhaustive compilation. A further listing of gene aliases and descriptions can be found using a variety of online databases, including GeneCards® (www.genecards.org), HUGO Gene Nomenclature (www.genenames.org), Entrez Gene (www.ncbi.nlm.nih.gov/entrez/query.fcgi?db=gene), UniProtKB/Swiss-Prot (www.uniprot.org), UniProtKB/TrEMBL (www.uniprot.org), OMIM (www.ncbi.nlm.nih.gov/entrez/query.fcgi?db=OMIM), GeneLoc (genecards.weizmann.acil/geneloc/), and Ensembl (www.ensembl.org). Generally, gene symbols and names below correspond to those approved by HUGO, and protein names are those recommended by UniProtKB/Swiss-Prot. Common alternatives are provided as well. Where a protein name indicates a precursor, the mature protein is also implied. Throughout the application, gene and protein symbols may be used interchangeably and the meaning can be derived from context, e.g., FISH is used to analyze nucleic acids whereas IHC is used to analyze protein.

TABLE 2
Gene and Protein Names

| Gene | | Protein | |
|---|---|---|---|
| Symbol | Gene Name | Symbol | Protein Name |
| ABCB1, | ATP-binding cassette, sub-family B | ABCB1, | Multidrug resistance protein 1; P- |
| PGP | (MDR/TAP), member 1 | MDR1, PGP | glycoprotein |
| ABCC1, | ATP-binding cassette, sub-family C | MRP1, | Multidrug resistance-associated protein |
| MRP1 | (CFTR/MRP), member 1 | ABCC1 | 1 |
| ABCG2, | ATP-binding cassette, sub-family G | ABCG2 | ATP-binding cassette sub-family G |
| BCRP | (WHITE), member 2 | | member 2 |
| ACE2 | angiotensin I converting enzyme | ACE2 | Angiotensin-converting enzyme 2 |
| | (peptidyl-dipeptidase A) 2 | | precursor |
| ADA | adenosine deaminase | ADA | Adenosine deaminase |
| ADH1C | alcohol dehydrogenase 1C (class I), | ADH1G | Alcohol dehydrogenase 1C |
| | gamma polypeptide | | |
| ADH4 | alcohol dehydrogenase 4 (class II), pi | ADH4 | Alcohol dehydrogenase 4 |
| | polypeptide | | |
| AGT | angiotensinogen (serpin peptidase | ANGT, AGT | Angiotensinogen precursor |
| | inhibitor, clade A, member 8) | | |
| ALK | anaplastic lymphoma receptor | ALK | ALK tyrosine kinase receptor precursor |
| | tyrosine kinase | | |
| AR | androgen receptor | AR | Androgen receptor |
| AREG | amphiregulin | AREG | Amphiregulin precursor |
| ASNS | asparagine synthetase | ASNS | Asparagine synthetase [glutamine- |
| | | | hydrolyzing] |
| BCL2 | B-cell CLL/lymphoma 2 | BCL2 | Apoptosis regulator Bcl-2 |
| BDCA1, | CD1c molecule | CD1C | T-cell surface glycoprotein CD1c |
| CD1C | | | precursor |
| BIRC5 | baculoviral IAP repeat-containing 5 | BIRC5, | Baculoviral IAP repeat-containing |
| | | Survivin | protein 5; Survivin |
| BRAF | v-raf murine sarcoma viral oncogene | B-RAF, | Serine/threonine-protein kinase B-raf |
| | homolog B1 | BRAF | |
| BRCA1 | breast cancer 1, early onset | BRCA1 | Breast cancer type 1 susceptibility |
| | | | protein |
| BRCA2 | breast cancer 2, early onset | BRCA2 | Breast cancer type 2 susceptibility |
| | | | protein |
| CA2 | carbonic anhydrase II | CA2 | Carbonic anhydrase 2 |
| CAV1 | caveolin 1, caveolae protein, 22 kDa | CAV1 | Caveolin-1 |
| CCND1 | cyclin D1 | CCND1, | G1/S-specific cyclin-D1 |
| | | Cyclin D1, | |
| | | BCL-1 | |
| CD20, | membrane-spanning 4-domains, | CD20 | B-lymphocyte antigen CD20 |
| MS4A1 | subfamily A, member 1 | | |
| CD25, | interleukin 2 receptor, alpha | CD25 | Interleukin-2 receptor subunit alpha |
| IL2RA | | | precursor |
| CD33 | CD33 molecule | CD33 | Myeloid cell surface antigen CD33 |
| | | | precursor |
| CD52, | CD52 molecule | CD52 | CAMPATH-1 antigen precursor |
| CDW52 | | | |
| CDA | cytidine deaminase | CDA | Cytidine deaminase |
| CDH1, | cadherin 1, type 1, E-cadherin | E-Cad | Cadherin-1 precursor (E-cadherin) |
| ECAD | (epithelial) | | |
| CDK2 | cyclin-dependent kinase 2 | CDK2 | Cell division protein kinase 2 |
| CDKN1A, | cyclin-dependent kinase inhibitor 1A | CDKN1A, | Cyclin-dependent kinase inhibitor 1 |
| P21 | (p21, Cip1) | p21 | |
| CDKN1B | cyclin-dependent kinase inhibitor 1B | CDKN1B, | Cyclin-dependent kinase inhibitor 1B |
| | (p27, Kip1) | p27 | |
| CDKN2A, | cyclin-dependent kinase inhibitor 2A | CD21A, p16 | Cyclin-dependent kinase inhibitor 2A, |
| P16 | (melanoma, p16, inhibits CDK.4) | | isoforms 1/2/3 |
| CES2 | carboxylesterase 2 (intestine, liver) | CES2, EST2 | Carboxylesterase 2 precursor |
| CK 5/6 | cytokeratin 5/cytokeratin 6 | CK 5/6 | Keratin, type II cytoskeletal 5; Keratin, |
| | | | type II cytoskeletal 6 |
| CK14, | keratin 14 | CK14 | Keratin, type I cytoskeletal 14 |
| KRT14 | | | |
| CK17, | keratin 17 | CK17 | Keratin, type I cytoskeletal 17 |
| KRT17 | | | |
| COX2, | prostaglandin-endoperoxide synthase | COX-2, | Prostaglandin G/H synthase 2 precursor |
| PTGS2 | 2 (prostaglandin G/H synthase and | PTGS2 | |
| | cyclooxygenase) | | |
| DCK | deoxycytidine kinase | DCK | Deoxycytidine kinase |
| DHFR | dihydrofolate reductase | DHFR | Dihydrofolate reductase |
| DNMT1 | DNA (cytosine-5-)-methyltransferase | DNMT1 | DNA (cytosine-5)-methyltransferase 1 |
| | 1 | | |
| DNMT3A | DNA (cytosine-5-)-methyltransferase | DNMT3A | DNA (cytosine-5)-methyltransferase 3A |
| | 3 alpha | | |
| DNMT3B | DNA (cytosine-5-)-methyltransferase | DNMT3B | DNA (cytosine-5)-methyltransferase 3B |
| | 3 beta | | |
| ECGF1, | thymidine phosphorylase | TYMP, PD- | Thymidine phosphorylase precursor |
| TYMP | | ECGF, | |
| | | ECDF1 | |
| EGFR, | epidermal growth factor receptor | EGFR, | Epidermal growth factor receptor |
| ERBB1, | (erythroblastic leukemia viral (v-erb- | ERBB1, | precursor |
| HER1 | b) oncogene homolog, avian) | HER1 | |
| EML4 | echinoderm microtubule associated | EML4 | Echinoderm microtubule-associated |
| | protein like 4 | | protein-like 4 |
| EPHA2 | EPH receptor A2 | EPHA2 | Ephrin type-A receptor 2 precursor |
| ER, ESR1 | estrogen receptor 1 | ER, ESR1 | Estrogen receptor |
| ERBB2, | v-erb-b2 erythroblastic leukemia | ERBB2, | Receptor tyrosine-protein kinase erbB-2 |
| HER2/NEU | viral oncogene homolog 2, | HER2, HER- | precursor |
| | neuro/glioblastoma derived oncogene | 2/neu | |
| | homolog (avian) | | |
| ERCC1 | excision repair cross-complementing | ERCC1 | DNA excision repair protein ERCC-1 |
| | rodent repair deficiency, | | |
| | complementation group 1 (includes | | |
| | overlapping antisense sequence) | | |
| ERCC3 | excision repair cross-complementing | ERCC3 | TFIIH basal transcription factor complex |
| | rodent repair deficiency, | | helicase XPB subunit |
| | complementation group 3 (xeroderma | | |
| | pigmentosum group B | | |
| | complementing) | | |
| EREG | Epiregulin | EREG | Proepiregulin precursor |
| FLT1 | fms-related tyrosine kinase 1 | FLT-1, | Vascular endothelial growth factor |
| | (vascular endothelial growth | VEGFR1 | receptor 1 precursor |
| | factor/vascular permeability factor | | |
| | receptor) | | |
| FOLR1 | folate receptor 1 (adult) | FOLR1 | Folate receptor alpha precursor |
| FOLR2 | folate receptor 2 (fetal) | FOLR2 | Folate receptor beta precursor |
| FSHB | follicle stimulating hormone, beta | FSHB | Follitropin subunit beta precursor |
| | polypeptide | | |
| FSHPRH1, | centromere protein I | FSHPRH1, | Centromere protein I |
| CENP1 | | CENP1 | |
| FSHR | follicle stimulating hormone | FSHR | Follicle-stimulating hormone receptor |
| | receptor | | precursor |
| FYN | FYN oncogene related to SRC, FGR, | FYN | Tyrosine-protein kinase Fyn |
| | YES | | |
| GART | phosphoribosylglycinamide | GART, | Trifunctional purine biosynthetic protein |
| | formyltransferase, | PUR2 | adenosine-3 |
| | phosphoribosylglycinamide | | |
| | synthetase, | | |
| | phosphoribosylaminoimidazole | | |
| | synthetase | | |
| GNA11, | guanine nucleotide binding protein | GNA11, G | Guanine nucleotide-binding protein |
| GA11 | (G protein), alpha 11 (Gq class) | alpha-11, G- | subunit alpha-11 |
| | | protein | |
| | | subunit | |
| | | alpha-11 | |
| GNAQ, | guanine nucleotide binding protein | GNAQ | Guanine nucleotide-binding protein G(q) |
| GAQ | (G protein), q polypeptide | | subunit alpha |
| GNRH1 | gonadotropin-releasing hormone 1 | GNRH1, | Progonadoliberin-1 precursor |
| | (luteinizing-releasing hormone) | GON1 | |
| GNRHR1, | gonadotropin-releasing hormone | GNRHR1 | Gonadotropin-releasing hormone |
| GNRHR | receptor | | receptor |
| GSTP1 | glutathione S-transferase pi 1 | GSTP1 | Glutathione S-transferase P |
| HCK | hemopoietic cell kinase | HCK | Tyrosine-protein kinase HCK |
| HDAC1 | histone deacetylase 1 | HDAC1 | Histone deacetylase 1 |
| HGF | hepatocyte growth factor | HGF | Hepatocyte growth factor precursor |
| | (hepapoietin A; scatter factor) | | |
| HIF1A | hypoxia inducible factor 1, alpha | HIF1A | Hypoxia-inducible factor 1-alpha |
| | subunit (basic helix-loop-helix | | |
| | transcription factor) | | |
| HIG1, | HIG1 hypoxia inducible domain | HIG1, | HIG1 domain family member 1A |
| HIGD1A, | family, member 1A | HIGD1A, | |
| HIG1A | | HIG1A | |
| HSP90AA1 | heat shock protein 90 kDa alpha | HSP90, | Heat shock protein HSP 90-alpha |
| HSP90, | (cytosolic), class A member 1 | HSP90A | |
| HSPCA | | | |
| IGF1R | insulin-like growth factor 1 receptor | IGF-1R | Insulin-like growth factor 1 receptor |
| | | | precursor |
| IGFBP3, | insulin-like growth factor binding | IGFBP-3, | Insulin-like growth factor-binding |
| IGFRBP3 | protein 3 | IBP-3 | protein 3 precursor |
| IGFBP4, | insulin-like growth factor binding | IGFBP-4, | Insulin-like growth factor-binding |
| IGFRBP4 | protein 4 | IBP-4 | protein 4 precursor |
| IGFBP5, | insulin-like growth factor binding | IGFBP-5, | Insulin-like growth factor-binding |
| IGFRBP5 | protein 5 | IBP-5 | protein 5 precursor |
| IL13RA1 | interleukin 13 receptor, alpha 1 | IL-13RA1 | Interleukin-13 receptor subunit alpha-1 |
| | | | precursor |
| KDR | kinase insert domain receptor (a type | KDR, | Vascular endothelial growth factor |
| | III receptor tyrosine kinase) | VEGFR2 | receptor 2 precursor |
| KIT, c-KIT | v-kit Hardy-Zuckerman 4 feline | KIT, c-KIT, | Mast/stem cell growth factor receptor |
| | sarcoma viral oncogene homolog | CD117, | precursor |
| | | SCFR | |
| KRAS | v-Ki-ras2 Kirsten rat sarcoma viral | K-RAS | GTPase KRas precursor |
| | oncogene homolog | | |
| LCK | lymphocyte-specific protein tyrosine | LCK | Tyrosine-protein kinase Lck |
| | kinase | | |
| LTB | lymphotoxin beta (TNF superfamily, | LTB, TNF3 | Lymphotoxin-beta |
| | member 3) | | |
| LTBR | lymphotoxin beta receptor (TNFR | LTBR, | Tumor necrosis factor receptor |
| | superfamily, member 3) | LTBR3, | superfamily member 3 precursor |
| | | TNFR | |
| LYN | v-yes-1 Yamaguchi sarcoma viral | LYN | Tyrosine-protein kinase Lyn |
| | related oncogene homolog | | |
| MET, c- | met proto-oncogene (hepatocyte | MET, c- | Hepatocyte growth factor receptor |
| MET | growth factor receptor) | MET | precursor |
| MGMT | O-6-methylguanine-DNA | MGMT | Methylated-DNA--protein-cysteine |
| | methyltransferase | | methyltransferase |
| MKI67, | antigen identified by monoclonal | Ki67, Ki-67 | Antigen KI-67 |
| KI67 | antibody Ki-67 | | |
| MLH1 | mutL homolog 1, colon cancer, | MLH1 | DNA mismatch repair protein Mlh1 |
| | nonpolyposis type 2 (E. coli) | | |
| MMR | mismatch repair (refers to MLH1, | | |
| | MSH2, MSH5) | | |
| MSH2 | mutS homolog 2, colon cancer, | MSH2 | DNA mismatch repair protein Msh2 |
| | nonpolyposis type 1 (E. coli) | | |
| MSH5 | mutS homolog 5 (E. coli) | MSH5, | MutS protein homolog 5 |
| | | hMSH5 | |
| MYC, c- | v-myc myelocytomatosis viral | MYC, c- | Myc proto-oncogene protein |
| MYC | oncogene homolog (avian) | MYC | |
| NBN, P95 | nibrin | NBN, p95 | Nibrin |
| NDGR1 | N-myc downstream regulated 1 | NDGR1 | Protein NDGR1 |
| NFKB1 | nuclear factor of kappa light | NFKB1 | Nuclear factor NF-kappa-B p105 |
| | polypeptide gene enhancer in B-cells | | subunit |
| | 1 | | |
| NFKB2 | nuclear factor of kappa light | NFKB2 | Nuclear factor NF-kappa-B p100 subunit |
| | polypeptide gene enhancer in B-cells | | |
| | 2 (p49/p100) | | |
| NFKBIA | nuclear factor of kappa light | NFKBIA | NF-kappa-B inhibitor alpha |
| | polypeptide gene enhancer in B-cells | | |
| | inhibitor, alpha | | |
| NRAS | neuroblastoma RAS viral (v-ras) | NRAS | GTPase NRas, Transforming protein N- |
| | oncogene homolog | | Ras |
| ODC1 | ornithine decarboxylase 1 | ODC | Ornithine decarboxylase |
| OGFR | opioid growth factor receptor | OGFR | Opioid growth factor receptor |
| PARP1 | poly (ADP-ribose) polymerase 1 | PARP-1 | Poly [ADP-ribose] polymerase 1 |
| PDGFC | platelet derived growth factor C | PDGF-C, | Platelet-derived growth factor C |
| | | VEGF-E | precursor |
| PDGFR | platelet-derived growth factor | PDGFR | Platelet-derived growth factor receptor |
| | receptor | | |
| PDGFRA | platelet-derived growth factor | PDGFRA, | Alpha-type platelet-derived growth |
| | receptor, alpha polypeptide | PDGFR2, | factor receptor precursor |
| | | CD140 A | |
| PDGFRB | platelet-derived growth factor | PDGFRB, | Beta-type platelet-derived growth factor |
| | receptor, beta polypeptide | PDGFR, | receptor precursor |
| | | PDGFR1, | |
| | | CD140 B | |
| PGR | progesterone receptor | PR | Progesterone receptor |
| PIK3CA | phosphoinositide-3-kinase, catalytic, | PI3K subunit | phosphoinositide-3-kinase, catalytic, |
| | alpha polypeptide | p110α | alpha polypeptide |
| POLA1 | polymerase (DNA directed), alpha 1, | POLA, | DNA polymerase alpha catalytic subunit |
| | catalytic subunit; polymerase (DNA | POLA1, | |
| | directed), alpha, polymerase (DNA | p180 | |
| | directed), alpha 1 | | |
| PPARG, | peroxisome proliferator-activated | PPARG | Peroxisome proliferator-activated |
| PPARG1, | receptor gamma | | receptor gamma |
| PPARG2, | | | |
| PPAR- | | | |
| gamma, | | | |
| NR1C3 | | | |
| PPARGC1A, | peroxisome proliferator-activated | PGC-1- | Peroxisome proliferator-activated |
| LEM6, | receptor gamma, coactivator 1 alpha | alpha, | receptor gamma coactivator 1-alpha; |
| PGC1, | | PPARGC-1- | PPAR-gamma coactivator 1-alpha |
| PGC1A, | | alpha | |
| PPARGC1 | | | |
| PSMD9, | proteasome (prosome, macropain) | p27 | 26S proteasome non-ATPase regulatory |
| P27 | 26S subunit, non-ATPase, 9 | | subunit 9 |
| PTEN, | phosphatase and tensin homolog | PTEN | Phosphatidylinositol-3,4,5-trisphosphate |
| MMAC1, | | | 3-phosphatase and dual-specificity |
| TEP1 | | | protein phosphatase; Mutated in multiple |
| | | | advanced cancers 1 |
| PTPN12 | protein tyrosine phosphatase, non- | PTPG1 | Tyrosine-protein phosphatase non- |
| | receptortype 12 | | receptor type 12; Protein-tyrosine |
| | | | phosphatase G1 |
| RAF1 | v-raf-1 murine leukemia viral | RAF, RAF- | RAF proto-oncogene serine/threonine- |
| | oncogene homolog 1 | 1, c-RAF | protein kinase |
| RARA | retinoic acid receptor, alpha | RAR, RAR- | Retinoic acid receptor alpha |
| | | alpha, | |
| | | RARA | |
| ROS1, | c-ros oncogene 1, receptor tyrosine | ROS1, ROS | Proto-oncogene tyrosine-protein kinase |
| ROS, | kinase | | ROS |
| MCF3 | | | |
| RRM1 | ribonucleotide reductase M1 | RRM1, RR1 | Ribonucleoside-diphosphate reductase |
| | | | large subunit |
| RRM2 | ribonucleotide reductase M2 | RRM2, | Ribonucleoside-diphosphate reductase |
| | | RR2M, RR2 | subunit M2 |
| RRM2B | ribonucleotide reductase M2 B (TP53 | RRM2B, | Ribonucleoside-diphosphate reductase |
| | inducible) | P53R2 | subunit M2 B |
| RXRB | retinoid X receptor, beta | RXRB | Retinoic acid receptor RXR-beta |
| RXRG | retinoid X receptor, gamma | RXRG, | Retinoic acid receptor RXR-gamma |
| | | RXRC | |
| SIK2 | salt-inducible kinase 2 | SIK2, | Salt-inducible protein kinase 2; |
| | | Q9H0K1 | Serine/threonine-protein kinase SIK2 |
| SLC29A1 | solute carrier family 29 (nucleoside | ENT-1 | Equilibrative nucleoside transporter 1 |
| | transporters), member 1 | | |
| SPARC | secreted protein, acidic, cysteine-rich | SPARC | SPARC precursor; Osteonectin |
| | (osteonectin) | | |
| SRC | v-src sarcoma (Schmidt-Ruppin A-2) | SRC | Proto-oncogene tyrosine-protein kinase |
| | viral oncogene homolog (avian) | | Src |
| SSTR1 | somatostatin receptor 1 | SSTR1, | Somatostatin receptor type 1 |
| | | SSR1, SS1R | |
| SSTR2 | somatostatin receptor 2 | SSTR2, | Somatostatin receptor type 2 |
| | | SSR2, SS2R | |
| SSTR3 | somatostatin receptor 3 | SSTR3, | Somatostatin receptor type 3 |
| | | SSR3, SS3R | |
| SSTR4 | somatostatin receptor 4 | SSTR4, | Somatostatin receptor type 4 |
| | | SSR4, SS4R | |
| SSTR5 | somatostatin receptor 5 | SSTR5, | Somatostatin receptor type 5 |
| | | SSR5, SS5R | |
| TK1 | thymidine kinase 1, soluble | TK1, KITH | Thymidine kinase, cytosolic |
| TLE3 | transducin-like enhancer of split 3 | TLE3 | Transducin-like enhancer protein 3 |
| | (E(sp1) homolog, Drosophila) | | |
| TNF | tumor necrosis factor (TNF | TNF, TNF- | Tumor necrosis factor precursor |
| | superfamily, member 2) | alpha, TNF-a | |
| TOP1, | topoisomerase (DNA) I | TOP1, | DNA topoisomerase 1 |
| TOPO1 | | TOPO1 | |
| TOP2A, | topoisomerase (DNA) II alpha | TOP2A, | DNA topoisomerase 2-alpha; |
| TOPO2A | 170 kDa | TOP2, | Topoisomerase II alpha |
| | | TOPO2A | |
| TOP2B, | topoisomerase (DNA) II beta | TOP2B, | DNA topoisomerase 2-beta; |
| TOPO2B | 180 kDa | TOPO2B | Topoisomerase II beta |
| TP53 | tumor protein p53 | p53 | Cellular tumor antigen p53 |
| TUBB3 | tubulin, beta 3 | Beta III | Tubulin beta-3 chain |
| | | tubulin, | |
| | | TUBB3, | |
| | | TUBB4 | |
| TXN | thioredoxin | TXN, TRX, | Thioredoxin |
| | | TRX-1 | |
| TXNRD1 | thioredoxin reductase 1 | TXNRD1, | Thioredoxin reductase 1, cytoplasmic; |
| | | TXNR | Oxidoreductase |
| TYMS, TS | thymidylate synthetase | TYMS, TS | Thymidylate synthase |
| VDR | vitamin D (1,25- dihydroxyvitamin | VDR | Vitamin D3 receptor |
| | D3) receptor | | |
| VEGFA, | vascular endothelial growth factor A | VEGF-A, | Vascular endothelial growth factor A |
| VEGF | | VEGF | precursor |
| VEGFC | vascular endothelial growth factor C | VEGF-C | Vascular endothelial growth factor C |
| | | | precursor |
| VHL | von Hippel-Lindau tumor suppressor | VHL | Von Hippel-Lindau disease tumor |
| | | | suppressor |
| YES1 | v-yes-1 Yamaguchi sarcoma viral | YES1, Yes, | Proto-oncogene tyrosine-protein kinase |
| | oncogene homolog 1 | p61-Yes | Yes |
| ZAP70 | zeta-chain (TCR) associated protein | ZAP-70 | Tyrosine-protein kinase ZAP-70 |
| | kinase 70 kDa | | |

In some embodiments, additional molecular profiling methods are performed. These can include without limitation PCR, RT-PCR, Q-PCR, SAGE, MPSS, immunoassays and other techniques to assess biological systems described herein or known to those of skill in the art. The choice of genes and gene products to be assayed can be updated over time as new treatments and new drug targets are identified. Once the expression or mutation of a biomarker is correlated with a treatment option, it can be assessed by molecular profiling. One of skill will appreciate that such molecular profiling is not limited to those techniques disclosed herein but comprises any methodology conventional for assessing nucleic acid or protein levels, sequence information, or both. The methods of the invention can also take advantage of any improvements to current methods or new molecular profiling techniques developed in the future. In some embodiments, a gene or gene product is assessed by a single molecular profiling technique. In other embodiments, a gene and/or gene product is assessed by multiple molecular profiling techniques. In a non-limiting example, a gene sequence can be assayed by one or more of FISH and pyrosequencing analysis, the mRNA gene product can be assayed by one or more of RT-PCR and microarray, and the protein gene product can be assayed by one or more of IHC and immunoassay. One of skill will appreciate that any combination of biomarkers and molecular profiling techniques that will benefit disease treatment are contemplated by the invention.

Genes and gene products that are known to play a role in cancer and can be assayed by any of the molecular profiling techniques of the invention include without limitation 2AR, A DISINTEGRIN, ACTIVATOR OF THYROID AND RETINOIC ACID RECEPTOR (ACTR), ADAM 11, ADIPOGENESIS INHIBITORY FACTOR (ADIF), ALPHA 6 INTEGRIN SUBUNIT, ALPHA V INTEGRIN SUBUNIT, ALPHA-CATENIN, AMPLIFIED IN BREAST CANCER 1 (AIB1), AMPLIFIED IN BREAST CANCER 3 (AIB3), AMPLIFIED IN BREAST CANCER 4 (AIB4), AMYLOID PRECURSOR PROTEIN SECRETASE (APPS), AP-2 GAMMA, APPS, ATP-BINDING CASSETTE TRANSPORTER (ABCT), PLACENTA-SPECIFIC (ABCP), ATP-BINDING CASSETTE SUBFAMILY C MEMBER (ABCC1), BAG-1, BASIGIN (BSG), BCEI, B-CELL DIFFERENTIATION FACTOR (BCDF), B-CELL LEUKEMIA 2 (BCL-2), B-CELL STIMULATORY FACTOR-2 (BSF-2), BCL-1, BCL-2-ASSOCIATED X PROTEIN (BAX), BCRP, BETA 1 INTEGRIN SUBUNIT, BETA 3 INTEGRIN SUBUNIT, BETA 5 INTEGRIN SUBUNIT, BETA-2 INTERFERON, BETA-CATENIN, BETA-CATENIN, BONE SIALOPROTEIN (BSP), BREAST CANCER ESTROGEN-INDUCIBLE SEQUENCE (BCEI), BREAST CANCER RESISTANCE PROTEIN (BCRP), BREAST CANCER TYPE 1 (BRCA1), BREAST CANCER TYPE 2 (BRCA2), BREAST CARCINOMA AMPLIFIED SEQUENCE 2 (BCAS2), CADHERIN, EPITHELIAL CADHERIN-11, CADHERIN-ASSOCIATED PROTEIN, CALCITONIN RECEPTOR (CTR), CALCIUM PLACENTAL PROTEIN (CAPL), CALCYCLIN, CALLA, CAMS, CAPL, CARCINOEMBRYONIC ANTIGEN (CEA), CATENIN, ALPHA 1, CATHEPSIN B, CATHEPSIN D, CATHEPSIN K, CATHEPSIN L2, CATHEPSIN 0, CATHEPSIN O1, CATHEPSIN V, CD10, CD146, CD147, CD24, CD29, CD44, CD51, CD54, CD61, CD66e, CD82, CD87, CD9, CEA, CELLULAR RETINOL-BINDING PROTEIN 1 (CRBP1), c-ERBB-2, CK7, CK8, CK18, CK19, CK20, CLAUDIN-7, c-MET, COLLAGENASE, FIBROBLAST, COLLAGENASE, INTERSTITIAL, COLLAGENASE-3, COMMON ACUTE LYMPHOCYTIC LEUKEMIA ANTIGEN (CALLA), CONNEXIN 26 (Cx26), CONNEXIN 43 (Cx43), CORTACTIN, COX-2, CTLA-8, CTR, CTSD, CYCLIN D1, CYCLOOXYGENASE-2, CYTOKERATIN 18, CYTOKERATIN 19, CYTOKERATIN 8, CYTOTOXIC T-LYMPHOCYTE-ASSOCIATED SERINE ESTERASE 8 (CTLA-8), DIFFERENTIATION-INHIBITING ACTIVITY (DIA), DNA AMPLIFIED IN MAMMARY CARCINOMA 1 (DAM1), DNA TOPOISOMERASE II ALPHA, DR-NM23, E-CADHERIN, EMMPRIN, EMS1, ENDOTHELIAL CELL GROWTH FACTOR (ECGR), PLATELET-DERIVED (PD-ECGF), ENKEPHALINASE, EPIDERMAL GROWTH FACTOR RECEPTOR (EGFR), EPISIALIN, EPITHELIAL MEMBRANE ANTIGEN (EMA), ER-ALPHA, ERBB2, ERBB4, ER-BETA, ERF-1, ERYTHROID-POTENTIATING ACTIVITY (EPA), ESR1, ESTROGEN RECEPTOR-ALPHA, ESTROGEN RECEPTOR-BETA, ETS-1, EXTRACELLULAR MATRIX METALLOPROTEINASE INDUCER (EMMPRIN), FIBRONECTIN RECEPTOR, BETA POLYPEPTIDE (FNRB), FIBRONECTIN RECEPTOR BETA SUBUNIT (FNRB), FLK-1, GA15.3, GA733.2, GALECTIN-3, GAMMA-CATENIN, GAP JUNCTION PROTEIN (26 kDa), GAP JUNCTION PROTEIN (43 kDa), GAP JUNCTION PROTEIN ALPHA-1 (GJA1), GAP JUNCTION PROTEIN BETA-2 (GJB2), GCP1, GELATINASE A, GELATINASE B, GELATINASE (72 kDa), GELATINASE (92 kDa), GLIOSTATIN, GLUCOCORTICOID RECEPTOR INTERACTING PROTEIN 1 (GRIP1), GLUTATHIONE S-TRANSFERASE p, GM-CSF, GRANULOCYTE CHEMOTACTIC PROTEIN 1 (GCP1), GRANULOCYTE-MACROPHAGE-COLONY STIMULATING FACTOR, GROWTH FACTOR RECEPTOR BOUND-7 (GRB-7), GSTp, HAP, HEAT-SHOCK COGNATE PROTEIN 70 (HSC70), HEAT-STABLE ANTIGEN, HEPATOCYTE GROWTH FACTOR (HGF), HEPATOCYTE GROWTH FACTOR RECEPTOR (HGFR), HEPATOCYTE-STIMULATING FACTOR III (HSF III), HER-2, HER2/NEU, HERMES ANTIGEN, HET, HHM, HUMORAL HYPERCALCEMIA OF MALIGNANCY (HHM), ICERE-1, INT-1, INTERCELLULAR ADHESION MOLECULE-1 (ICAM-1), INTERFERON-GAMMA-INDUCING FACTOR (IGIF), INTERLEUKIN-1 ALPHA (IL-1A), INTERLEUKIN-1 BETA (IL-1B), INTERLEUKIN-11 (IL-11), INTERLEUKIN-17 (IL-17), INTERLEUKIN-18 (IL-18), INTERLEUKIN-6 (IL-6), INTERLEUKIN-8 (IL-8), INVERSELY CORRELATED WITH ESTROGEN RECEPTOR EXPRESSION-1 (ICERE-1), KAI1, KDR, KERATIN 8, KERATIN 18, KERATIN 19, KISS-1, LEUKEMIA INHIBITORY FACTOR (LIF), LIF, LOST IN INFLAMMATORY BREAST CANCER (LIBC), LOT (“LOST ON TRANSFORMATION”), LYMPHOCYTE HOMING RECEPTOR, MACROPHAGE-COLONY STIMULATING FACTOR, MAGE-3, MAMMAGLOBIN, MASPIN, MC56, M-CSF, MDC, MDNCF, MDR, MELANOMA CELL ADHESION MOLECULE (MCAM), MEMBRANE METALLOENDOPEPTIDASE (MME), MEMBRANE-ASSOCIATED NEUTRAL ENDOPEPTIDASE (NEP), CYSTEINE-RICH PROTEIN (MDC), METASTASIN (MTS-1), MLN64, MMP1, MMP2, MMP3, MMP1, MMP9, MMP11, MMP13, MMP14, MMP15, MMP16, MMP17, MOESIN, MONOCYTE ARGININE-SERPIN, MONOCYTE-DERIVED NEUTROPHIL CHEMOTACTIC FACTOR, MONOCYTE-DERIVED PLASMINOGEN ACTIVATOR INHIBITOR, MTS-1, MUC-1, MUC18, MUCIN LIKE CANCER ASSOCIATED ANTIGEN (MCA), MUCIN, MUC-1, MULTIDRUG RESISTANCE PROTEIN 1 (MDR, MDR1), MULTIDRUG RESISTANCE RELATED PROTEIN-1 (MRP, MRP-1), N-CADHERIN, NEP, NEU, NEUTRAL ENDOPEPTIDASE, NEUTROPHIL-ACTIVATING PEPTIDE 1 (NAP1), NM23-H1, NM23-H2, NME1, NME2, NUCLEAR RECEPTOR COACTIVATOR-1 (NCoA-1), NUCLEAR RECEPTOR COACTIVATOR-2 (NCoA-2), NUCLEAR RECEPTOR COACTIVATOR-3 (NCoA-3), NUCLEOSIDE DIPHOSPHATE KINASE A (NDPKA), NUCLEOSIDE DIPHOSPHATE KINASE B (NDPKB), ONCOSTATIN M (OSM), ORNITHINE DECARBOXYLASE (ODC), OSTEOCLAST DIFFERENTIATION FACTOR (ODF), OSTEOCLAST DIFFERENTIATION FACTOR RECEPTOR (ODFR), OSTEONECTIN (OSN, ON), OSTEOPONTIN (OPN), OXYTOCIN RECEPTOR (OXTR), p27/kipl, p300/CBP COINTEGRATOR ASSOCIATE PROTEIN (p/CIP), p53, p9Ka, PAI-1, PAI-2, PARATHYROID ADENOMATOSIS 1 (PRAD1), PARATHYROID HORMONE-LIKE HORMONE (PTHLH), PARATHYROID HORMONE-RELATED PEPTIDE (PTHrP), P-CADHERIN, PD-ECGF, PDGF, PEANUT-REACTIVE URINARY MUCIN (PUM), P-GLYCOPROTEIN (P-GP), PGP-1, PHGS-2, PHS-2, PIP, PLAKOGLOBIN, PLASMINOGEN ACTIVATOR INHIBITOR (TYPE 1), PLASMINOGEN ACTIVATOR INHIBITOR (TYPE 2), PLASMINOGEN ACTIVATOR (TISSUE-TYPE), PLASMINOGEN ACTIVATOR (UROKINASE-TYPE), PLATELET GLYCOPROTEIN IIIa (GP3A), PLAU, PLEOMORPHIC ADENOMA GENE-LIKE 1 (PLAGL1), POLYMORPHIC EPITHELIAL MUCIN (PEM), PRAD1, PROGESTERONE RECEPTOR (PgR), PROGESTERONE RESISTANCE, PROSTAGLANDIN ENDOPEROXIDE SYNTHASE-2, PROSTAGLANDIN G/H SYNTHASE-2, PROSTAGLANDIN H SYNTHASE-2, pS2, PS6K, PSORIASIN, PTHLH, PTHrP, RAD51, RAD52, RAD54, RAP46, RECEPTOR-ASSOCIATED COACTIVATOR 3 (RAC3), REPRESSOR OF ESTROGEN RECEPTOR ACTIVITY (REA), S100A4, S100A6, S100A7, S6K, SART-1, SCAFFOLD ATTACHMENT FACTOR B (SAF-B), SCATTER FACTOR (SF), SECRETED PHOSPHOPROTEIN-1 (SPP-1), SECRETED PROTEIN, ACIDIC AND RICH IN CYSTEINE (SPARC), STANNICALCIN, STEROID RECEPTOR COACTIVATOR-1 (SRC-1), STEROID RECEPTOR COACTIVATOR-2 (SRC-2), STEROID RECEPTOR COACTIVATOR-3 (SRC-3), STEROID RECEPTOR RNA ACTIVATOR (SRA), STROMELYSIN-1, STROMELYSIN-3, TENASCIN-C(TN-C), TESTES-SPECIFIC PROTEASE 50, THROMBOSPONDIN I, THROMBOSPONDIN II, THYMIDINE PHOSPHORYLASE (TP), THYROID HORMONE RECEPTOR ACTIVATOR MOLECULE 1 (TRAM-1), TIGHT JUNCTION PROTEIN 1 (TJP1), TIMP1, TIMP2, TIMP3, TIMP4, TISSUE-TYPE PLASMINOGEN ACTIVATOR, TN-C, TP53, IPA, TRANSCRIPTIONAL INTERMEDIARY FACTOR 2 (TIF2), TREFOIL FACTOR 1 (TFF1), TSG101, TSP-1, TSP1, TSP-2, TSP2, TSP50, TUMOR CELL COLLAGENASE STIMULATING FACTOR (TCSF), TUMOR-ASSOCIATED EPITHELIAL MUCIN, uPA, uPAR, UROKINASE, UROKINASE-TYPE PLASMINOGEN ACTIVATOR, UROKINASE-TYPE PLASMINOGEN ACTIVATOR RECEPTOR (uPAR), UVOMORULIN, VASCULAR ENDOTHELIAL GROWTH FACTOR, VASCULAR ENDOTHELIAL GROWTH FACTOR RECEPTOR-2 (VEGFR2), VASCULAR ENDOTHELIAL GROWTH FACTOR-A, VASCULAR PERMEABILITY FACTOR, VEGFR2, VERY LATE T-CELL ANTIGEN BETA (VLA-BETA), VIMENTIN, VITRONECTIN RECEPTOR ALPHA POLYPEPTIDE (VNRA), VITRONECTIN RECEPTOR, VON WILLEBRAND FACTOR, VPF, VWF, WNT-1, ZAC, ZO-1, and ZONULA OCCLUDENS-1.

The gene products used for IHC expression profiling include without limitation one or more of AR, BCRP, BCRP1, BRCA1, CAV-1, CK 5/6, CK14, CK17, c-Kit, cMET, cMYC, COX2, Cyclin D1, ECAD, EGFR, ER, ERCC1, Her2/Neu, IGF1R, IGFRBP1, IGFRBP2, IGFRBP3, IGFRBP4, IGFRBP5, IGFRBP6, IGFRBP7, Ki67, MGMT, MRP1, P53, P95, PDGFR, PDGFRA, PGP (MDR1), PR, PTEN, RRM1, SPARC, TLE3, TOP1, TOP2, TOP2A, TS, and TUBB3. In an embodiment, the IHC is performed on AR, BCRP, CAV-1, CK 5/6, CK14, CK17, c-Kit, COX2, Cyclin D1, ECAD, EGFR, ER, ERCC1, Her2/Neu, IGF1R, Ki67, MGMT, MRP1, P53, P95, PDGFRa, PGP (MDR1), PR, PTEN, RRM1, SPARC, TLE3, TOP1, TOP2A, TS, and TUBB3. In some embodiments, IHC analysis includes one or more of c-Met, EML4-ALK fusion, KENT-1, IGF-1R, MMR, p16, p21, p27, PARP-1, PI3K, and TLE3. IHC profiling of EGFR can also be performed. IHC is also used to detect or test for various gene products, including without limitation one or more of the following: EGFR, SPARC, C-kit, ER, PR, Androgen receptor, PGP, RRM1, TOPO1, BRCP1, MRP1, MGMT, PDGFR, DCK, ERCC1, Thymidylate synthase, Her2/neu, or TOPO2A. In some embodiments, IHC is used to detect on or more of the following proteins, including without limitation: ADA, AR, ASNA, BCL2, BRCA2, c-Met, CD33, CDW52, CES2, DNMT1, EGFR, EML4-ALK fusion, ERBB2, ERCC3, ESR1, FOLR2, GART, GSTP1, HDAC1, hENT-1, HIF1A, HSPCA, IGF-1R, IL2RA, KIT, MLH1, MMR, MS4A1, MASH2, NFKB2, NFKBIA, OGFR, p16, p21, p27, PARP-1, PI3K, PDGFC, PDGFRA, PDGFRB, PGR, POLA, PTEN, PTGS2, RAF1, RARA, RXRB, SPARC, SSTR1, TK1, TLE3, TNF, TOP1, TOP2A, TOP2B, TXNRD1, TYMS, VDR, VEGF, VHL, or ZAP70. The proteins can be detected by IHC using monoclonal or polyclonal antibodies. In some embodiments, both are used. As an illustrative example, SPARC can be detected by anti-SPARC monoclonal (SPARC mono, SPARC m) and/or anti-SPARC polyclonal (SPARC poly, SPARC p) antibodies.

In some embodiments, IHC analysis according to the methods of the invention includes one or more of AR, c-Kit, COX2, CAV-1, CK 5/6, CK14, CK17, ECAD, ER, Her2/Neu, Ki67, MRP1, P53, PDGFR, PGP, PR, PTEN, SPARC, TLE3 and TS. All of these genes can be examined. As indicated by initial results of IHC or other molecular profiling methods as described herein, additional IHC assayscan be performed. In one embodiment, the additional IHC comprises that of p95, or p95, Cyclin D1 and EGFR. IHC can also be performed on IGFRBP3, IGFRBP4, IGFRBP5, or other forms of IGFRBP (e.g., IGFRBP1, IGFRBP2, IGFRBP6, IGFRBP7). In another embodiment, the additional IHC comprises that of one or more of BCRP, ERCC1, MGMT, P95, RRM1, TOP2A, and TOP1. In still another embodiment, the additional IHC comprises that of one or more of BCRP, Cyclin D1, EGFR, ERCC1, MGMT, P95, RRM1, TOP2A, and TOP1. Any useful subset or all of these genes can be examined. The additional IHC can be selected on the basis of molecular characteristics of the tumor so that IHC is only performed where it is likely to indicate a candidate therapy for treating the cancer. As described herein, the molecular characteristics of the tumor determined can be determined by IHC combined with one or more of FISH, DNA microarray and mutation analysis. The genes and/or gene products used for IHC analysis can be at least 1, 2, 3, 4, 5, 6, 7, 8, 9, 10, 15, 20, 25, 30, 40, 50, 60, 70, 80, 90, 100 or all of the genes and/or gene products listed in Table 2.

Microarray expression profiling can be used to simultaneously measure the expression of one or more genes or gene products, including without limitation ABCC1, ABCG2, ADA, AR, ASNS, BCL2, BIRC5, BRCA1, BRCA2, CD33, CD52, CDA, CES2, DCK, DHFR, DNMT1, DNMT3A, DNMT3B, ECGF1, EGFR, EPHA2, ERBB2, ERCC1, ERCC3, ESR1, FLT1, FOLR2, FYN, GART, GNRH1, GSTP1, HCK, HDAC1, HIF1A, HSP90AA1, IGFBP3, IGFBP4, IGFBP5, IL2RA, KDR, KIT, LCK, LYN, MET, MGMT, MLH1, MS4A1, MSH2, NFKB1, NFKB2, NFKBIA, OGFR, PARP1, PDGFC, PDGFRA, PDGFRB, PGP, PGR, POLA1, PTEN, PTGS2, RAF1, RARA, RRM1, RRM2, RRM2B, RXRB, RXRG, SIK2, SPARC, SRC, SSTR1, SSTR2, SSTR3, SSTR4, SSTR5, TK1, TNF, TOP1, TOP2A, TOP2B, TXNRD1, TYMS, VDR, VEGFA, VHL, YES1, and ZAP70. In some embodiments, the genes used for the microarray expression profiling comprise one or more of: EGFR, SPARC, C-kit, ER, PR, Androgen receptor, PGP, RRM1, TOPO1, BRCP1, MRP1, MGMT, PDGFR, DCK, ERCC1, Thymidylate synthase, Her2/neu, TOPO2A, ADA, AR, ASNA, BCL2, BRCA2, CD33, CDW52, CES2, DNMT1, EGFR, ERBB2, ERCC3, ESR1, FOLR2, GART, GSTP1 HDAC1, HIF1A, HSPCA, IL2RA, KIT, MLH1, MS4A1, MASH2, NFKB2, NFKBIA, OGFR, PDGFC, PDGFRA, PDGFRB, PGR, POLA, PTEN, PTGS2, RAF1, RARA, RXRB, SPARC, SSTR1, TK1, TNF, TOP1, TOP2A, TOP2B, TXNRD1, TYMS, VDR, VEGF, VHL, and ZAP70. One or more of the following genes can also be assessed by microarray expression profiling: ALK, EML4, hENT-1, IGF-IR, HSP90AA1, MMR, p16, p21, p27, PARD-1, PI3K and TLE3. The microarray expression profiling can be performed using a low density microarray, an expression microarray, a comparative genomic hybridization (CGH) microarray, a single nucleotide polymorphism (SNP) microarray, a proteomic array an antibody array, or other array as disclosed herein or known to those of skill in the art. In some embodiments, high throughput expression arrays are used. Such systems include without limitation commercially available systems from Affymetrix, Agilent or Illumina, as described in more detail herein.

Microarray expression profiling can be used to simultaneously measure the expression of one or more genes or gene products, including without limitation ABCC1, ABCG2, ADA, AR, ASNS, BCL2, BIRC5, BRCA1, BRCA2, CD33, CD52, CDA, CES2, DCK, DHFR, DNMT1, DNMT3A, DNMT3B, ECGF1, EGFR, EPHA2, ERBB2, ERCC1, ERCC3, ESR1, FLT1, FOLR2, FYN, GART, GNRH1, GSTP1, HCK, HDAC1, HIF1A, HSP90AA1, IGFBP3, IGFBP4, IGFBP5, IL2RA, KDR, KIT, LCK, LYN, MET, MGMT, MLH1, MS4A1, MSH2, NFKB1, NFKB2, NFKBIA, OGFR, PARP1, PDGFC, PDGFRA, PDGFRB, PGP, PGR, POLA1, PTEN, PTGS2, PTPN12, RAF1, RARA, RRM1, RRM2, RRM2B, RXRB, RXRG, SIK2, SPARC, SRC, SSTR1, SSTR2, SSTR3, SSTR4, SSTR5, TK1, TNF, TOP1, TOP2A, TOP2B, TXNRD1, TYMS, VDR, VEGFA, VHL, YES1, and ZAP70. The genes and/or gene products used for RT-PCR profiling analysis can be at least 1, 2, 3, 4, 5, 6, 7, 8, 9, 10, 15, 20, 25, 30, 40, 50, 60, 70, 80, 90, 100 or all of the genes and/or gene products listed in Table 2.

Expression profiling can be performed using PCR, e.g., real-time PCR (qPCR or RT-PCR). RT-PCR can be used to measure the expression of one or more genes or gene products, including without limitation ABCC1, ABCG2, ACE2, ADA, ADH1C, ADH4, AGT, AR, AREG, ASNS, BCL2, BCRP, BDCA1, beta III tubulin, BIRC5, B-RAF, BRCA1, BRCA2, CA2, caveolin, CD20, CD25, CD33, CD52, CDA, CDKN2A, CDKNIA, CDKN1B, CDK2, CDW52, CES2, CK 14, CK 17, CK 5/6, c-KIT, c-Met, c-Myc, COX-2, Cyclin D1, DCK, DHFR, DNMT1, DNMT3A, DNMT3B, E-Cadherin, ECGF1, EGFR, EML4-ALK fusion, EPHA2, Epiregulin, ER, ERBR2, ERCC1, ERCC3, EREG, ESR1, FLT1, folate receptor, FOLR1, FOLR2, FSHB, FSHPRH1, FSHR, FYN, GART, GNA11, GNAQ, GNRH1, GNRHR1, GSTP1, HCK, HDAC1, hENT-1, Her2/Neu, HGF, HIF1A, HIG1, HSP90, HSP90AA1, HSPCA, IGF-1R, IGFRBP, IGFRBP3, IGFRBP4, IGFRBP5, IL13RA1, IL2RA, KDR, Ki67, KIT, K-RAS, LCK, LTB, Lymphotoxin Beta Receptor, LYN, MET, MGMT, MLH1, MMR, MRP1, MS4A1, MSH2, MSH5, Myc, NFKB1, NFKB2, NFKBIA, NRAS, ODC1, OGFR, p16, p21, p27, p53, p95, PARP-1, PDGFC, PDGFR, PDGFRA, PDGFRB, PGP, PGR, PI3K, POLA, POLA1, PPARG, PPARGC1, PR, PTEN, PTGS2, PTPN12, RAF1, RARA, ROS1, RRM1, RRM2, RRM2B, RXRB, RXRG, SIK2, SPARC, SRC, SSTR1, SSTR2, SSTR3, SSTR4, SSTR5, Survivin, TK1, TLE3, TNF, TOP1, TOP2A, TOP2B, TS, TUBB3, TXN, TXNRD1, TYMS, VDR, VEGF, VEGFA, VEGFC, VHL, YES1, ZAP70. For example, the genes assessed by RT-PCR can include AREG, BRCA1, EGFR, ERBB3, ERCC1, EREG, PGP (MDR-1), RRM1, TOPO1, TOPO2A, TS, TUBB3 and VEGFR2. The genes and/or gene products used for real-time PCR analysis can be at least 1, 2, 3, 4, 5, 6, 7, 8, 9, 10, 15, 20, 25, 30, 40, 50, 60, 70, 80, 90, 100 or all of the genes and/or gene products listed in Table 2. The PCR can be performed in a high throughput fashion, e.g., using multiplex amplification, microfluidics, and/or using a low density microarray.

FISH analysis can be used to profile one or more of HER2, CMET, PIK3CA, EGFR, TOP2A, CMYC and EML4-ALK fusion. In some embodiments, FISH is used to detect or test for one or more of the following genes, including without limitation: EGFR, SPARC, C-kit, ER, PR, AR, PGP, RRM1, TOPO1, BRCP1, MRP1, MGMT, PDGFR, DCK, ERCC1, TS, HER2, or TOPO2A. In some embodiments, FISH is used to detect or test for one or more of EML4-ALK fusion and IGF-1R. In some embodiments, FISH is used to detect or test various biomarkers, including without limitation one or more of the following: ADA, AR, ASNA, BCL2, BRCA2, c-Met, CD33, CDW52, CES2, DNMT1, EGFR, EML4-ALK fusion, ERBB2, ERCC3, ESR1, FOLR2, GART, GSTP1, HDAC1, hENT-1, HIF1A, HSPCA, IGF-1R, IL2RA, KIT, MLH1, MMR, MS4A1, MASH2, NFKB2, NFKBIA, OGFR, p16, p21, p27, PARP-1, PI3K, PDGFC, PDGFRA, PDGFRB, PGR, POLA, PTEN, PTGS2, RAF1, RARA, RXRB, SPARC, SSTR1, TK1, TLE3, TNF, TOP1, TOP2A, TOP2B, TXNRD1, TYMS, VDR, VEGF, VHL, or ZAP70.

In some embodiments, FISH is used to detect or test for HER2, and depending on the results of the HER2 analysis and other molecular profiling techniques, additional FISH testing may be performed. The additional FISH testing can comprise that of CMYC and/or TOP2A. For example, FISH testing may indicate that a cancer is HER2+. The cancer may be a breast cancer. HER2+ cancers may then be followed up by FISH testing for CMYC and TOP2A, whereas HER2− cancers are followed up with FISH testing for CMYC. For some cancers, e.g., triple negative breast cancer (i.e., ER−/PR−/HER2−), additional FISH testing may not be performed. The decision whether to perform additional FISH testing can be guided by whether the additional FISH testing is likely to reveal information about candidate therapies for the cancer. The additional FISH can be selected on the basis of molecular characteristics of the tumor so that FISH is only performed where it is likely to indicate a candidate therapy for treating the cancer. As described herein, the molecular characteristics of the tumor determined can be determined by one or more of IHC, FISH, DNA microarray and sequence analysis. The genes and/or gene products used for FISH analysis can be at least 1, 2, 3, 4, 5, 6, 7, 8, 9, 10, 15, 20, 25, 30, 40, 50, 60, 70, 80, 90, 100 or all of the genes and/or gene products listed in Table 2.

In some embodiments, the genes used for the mutation profiling comprise one or more of PIK3CA, EGFR, cKIT, KRAS, NRAS and BRAF. Mutation profiling can be determined by sequencing, including Sanger sequencing, array sequencing, pyrosequencing, NextGen sequencing, etc. Sequence analysis may reveal that genes harbor activating mutations so that drugs that inhibit activity are indicated for treatment. Alternately, sequence analysis may reveal that genes harbor mutations that inhibit or eliminate activity, thereby indicating treatment for compensating therapies. In embodiments, sequence analysis comprises that of exon 9 and 11 of c-KIT. Sequencing may also be performed on EGFR-kinase domain exons 18, 19, 20, and 21. Mutations, amplifications or misregulations of EGFR or its family members are implicated in about 30% of all epithelial cancers. Sequencing can also be performed on PI3K, encoded by the PIK3CA gene. This gene is a found mutated in many cancers. Sequencing analysis can also comprise assessing mutations in one or more ABCC1, ABCG2, ADA, AR, ASNS, BCL2, BIRC5, BRCA1, BRCA2, CD33, CD52, CDA, CES2, DCK, DHFR, DNMT1, DNMT3A, DNMT3B, ECGF1, EGFR, EPHA2, ERBB2, ERCC1, ERCC3, ESR1, FLT1, FOLR2, FYN, GART, GNRH1, GSTP1, HCK, HDAC1, HIF1A, HSP90AA1, IGFBP3, IGFBP4, IGFBP5, IL2RA, KDR, KIT, LCK, LYN, MET, MGMT, MLH1, MS4A1, MSH2, NFKB1, NFKB2, NFKBIA, NRAS, OGFR, PARP1, PDGFC, PDGFRA, PDGFRB, PGP, PGR, POLA1, PTEN, PTGS2, PTPN12, RAF1, RARA, RRM1, RRM2, RRM2B, RXRB, RXRG, SIK2, SPARC, SRC, SSTR1, SSTR2, SSTR3, SSTR4, SSTR5, TK1, TNF, TOP1, TOP2A, TOP2B, TXNRD1, TYMS, VDR, VEGFA, VHL, YES1, and ZAP70. One or more of the following genes can also be assessed by sequence analysis: ALK, EML4, hENT-1, IGF-1R, HSP90AA1, MMR, p16, p21, p27, PARP-1, PI3K and TLE3. The genes and/or gene products used for mutation or sequence analysis can be at least 1, 2, 3, 4, 5, 6, 7, 8, 9, 10, 15, 20, 25, 30, 40, 50, 60, 70, 80, 90, 100 or all of the genes and/or gene products listed in Table 2, Table 6 or Table 25.

In some embodiments, mutational analysis is performed on PIK3CA. The decision whether to perform mutational analysis on PIK3CA can be guided by whether this testing is likely to reveal information about candidate therapies for the cancer. The PIK3CA mutational analysis can be selected on the basis of molecular characteristics of the tumor so that the analysis is only performed where it is likely to indicate a candidate therapy for treating the cancer. As described herein, the molecular characteristics of the tumor determined can be determined by one or more of IHC, FISH, DNA microarray and sequence analysis. In one embodiment, PIK3CA is analyzed for a HER2+ cancer. The cancer can be a breast cancer.

In a related aspect, the invention provides a method of identifying a candidate treatment for a subject in need thereof by using molecular profiling of sets of known biomarkers. For example, the method can identify a chemotherapeutic agent for an individual with a cancer. The method comprises: obtaining a sample from the subject; performing an immunohistochemistry (IHC) analysis on the sample to determine an IHC expression profile on one or more, e.g. 2, 3, 4, 5, 6, 7, 8, 9, 10 or more, of: SPARC, PGP, Her2/neu, ER, PR, c-kit, AR, CD52, PDGFR, TOP2A, TS, ERCC1, RRM1, BCRP, TOPO1, PTEN, MGMT, MRP1, c-Met, EML4-ALK fusion, hENT-1, IGF-1R, MMR, p16, p21, p27, PARP-1, PI3K, COX2 and TLE3; performing a microarray analysis on the sample to determine a microarray expression profile on one or more, e.g. 2, 3, 4, 5, 6, 7, 8, 9, 10 or more, of: ABCC1, ABCG2, ADA, AR, ASNS, BCL2, BIRC5, BRCA1, BRCA2, CD33, CD52, CDA, CES2, DCK, DHFR, DNMT1, DNMT3A, DNMT3B, ECGF1, EGFR, EPHA2, ERBB2, ERCC1, ERCC3, ESR1, FLT1, FOLR2, FYN, GART, GNRH1, GSTP1, HCK, HDAC1, HIF1A, HSP90AA1, IGFBP3, IGFBP4, IGFBP5, IL2RA, KDR, KIT, LCK, LYN, MET, MGMT, MLH1, MS4A1, MSH2, NFKB1, NFKB2, NFKBIA, OGFR, PARP1, PDGFC, PDGFRA, PDGFRB, PGP, PGR, POLA1, PTEN, PTGS2, PTPN12, RAF1, RARA, RRM1, RRM2, RRM2B, RXRB, RXRG, SIK2, SPARC, SRC, SSTR1, SSTR2, SSTR3, SSTR4, SSTR5, TK1, TNF, TOP1, TOP2A, TOP2B, TXNRD1, TYMS, VDR, VEGFA, VHL, YES1, and ZAP70; performing a fluorescent in-situ hybridization (FISH) analysis on the sample to determine a FISH mutation profile on at least one of EGFR, HER2, EML4-ALK fusion and IGF-1R; performing DNA sequencing on the sample to determine a sequencing mutation profile on at least one of KRAS, BRAF, c-KIT, PI3K (PIK3CA), NRAS and EGFR; and comparing the IHC expression profile, microarray expression profile, FISH mutation profile and sequencing mutation profile against a rules database, wherein the rules database comprises a mapping of treatments whose biological activity is known against diseased cells that: i) overexpress or underexpress one or more proteins included in the IHC expression profile; ii) overexpress or underexpress one or more genes included in the microarray expression profile; iii) have zero or more mutations in one or more genes included in the FISH mutation profile; and/or iv) have zero or more mutations in one or more genes included in the sequencing mutation profile; and identifying the treatment if the comparison against the rules database indicates that the treatment should have biological activity against the disease; and the comparison against the rules database does not contraindicate the treatment for treating the disease. The disease can be a cancer. The molecular profiling steps can be performed in any order. In some embodiments, not all of the molecular profiling steps are performed. As a non-limiting example, microarray analysis is not performed if the sample quality does not meet a threshold value, as described herein. In some embodiments, the IHC expression profiling is performed on at least 20%, 30%, 40%, 50%, 60%, 70%, 80%, 90%, or 95% of the gene products above. In some embodiments, the microarray expression profiling is performed on at least 20%, 30%, 40%, 50%, 60%, 70%, 80%, 90%, or 95% of the genes listed above. In some embodiments, the IHC expression profiling is performed on all of the gene products above. In some embodiments, the microarray profiling is performed on all of the genes listed above. In some embodiments, the FISH profiling is performed on all of the gene products above. In some embodiments, the sequence profiling is performed on all of the genes listed above.

In a related aspect, the invention provides a method of identifying a candidate treatment for a subject in need thereof by using molecular profiling of defined sets of known biomarkers. For example, the method can identify a chemotherapeutic agent for an individual with a cancer. The method comprises: obtaining a sample from the subject, wherein the sample comprises formalin-fixed paraffin-embedded (FFPE) tissue or fresh frozen tissue, and wherein the sample comprises cancer cells; performing an immunohistochemistry (IHC) analysis on the sample to determine an IHC expression profile on at least: SPARC, PGP, Her2/neu, ER, PR, c-kit, AR, CD52, PDGFR, TOP2A, TS, ERCC1, RRM1, BCRP, TOPO1, PTEN, MGMT, MRP1, c-Met, EML4-ALK fusion, hENT-1, IGF-1R, MMR, p16, p21, p27, PARP-1, PI3K, and TLE3; performing a microarray analysis on the sample to determine a microarray expression profile on at least: ABCC1, ABCG2, ADA, AR, ASNS, BCL2, BIRC5, BRCA1, BRCA2, CD33, CD52, CDA, CES2, DCK, DHFR, DNMT1, DNMT3A, DNMT3B, ECGF1, EGFR, EPHA2, ERBB2, ERCC1, ERCC3, ESR1, FLT1, FOLR2, FYN, GART, GNRH1, GSTP1, HCK, HDAC1, HIFIA, HSP90AA1, IGFBP3, IGFBP4, IGFBP5, IL2RA, KDR, KIT, LCK, LYN, MET, MGMT, MLH1, MS4A1, MSH2, NFKB1, NFKB2, NFKBIA, OGFR, PARP1, PDGFC, PDGFRA, PDGFRB, PGP, PGR, POLA1, PTEN, PTGS2, PTPN12, RAF1, RARA, RRM1, RRM2, RRM2B, RXRB, RXRG, SIK2, SPARC, SRC, SSTR1, SSTR2, SSTR3, SSTR4, SSTR5, TK1, TNF, TOP1, TOP2A, TOP2B, TXNRD1, TYMS, VDR, VEGFA, VHL, YES1, and ZAP70; performing a fluorescent in-situ hybridization (FISH) analysis on the sample to determine a FISH mutation profile on at least one of EGFR, HER2, EML4-ALK fusion and IGF-1R; performing DNA sequencing on the sample to determine a sequencing mutation profile on at least KRAS, BRAF, c-KIT, PI3K (PIK3CA), NRAS and EGFR. The IHC expression profile, microarray expression profile, FISH mutation profile and sequencing mutation profile are compared against a rules database, wherein the rules database comprises a mapping of treatments whose biological activity is known against diseased cells that: i) overexpress or underexpress one or more proteins included in the IHC expression profile; ii) overexpress or underexpress one or more genes included in the microarray expression profile; iii) have zero or more mutations in one or more genes included in the FISH mutation profile; or iv) have zero or more mutations in one or more genes included in the sequencing mutation profile; and identifying the treatment if the comparison against the rules database indicates that the treatment should have biological activity against the disease; and the comparison against the rules database does not contraindicate the treatment for treating the disease. The disease can be a cancer. The molecular profiling steps can be performed in any order. In some embodiments, not all of the molecular profiling steps are performed. As a non-limiting example, microarray analysis is not performed if the sample quality does not meet a threshold value, as described herein. In some embodiments, the biological material is mRNA and the quality control test comprises a A260/A280 ratio and/or a Ct value of RT-PCR using a housekeeping gene, e.g., RPL13a. In embodiments, the mRNA does not pass the quality control test if the A260/A280 ratio <1.5 or the RPL13a Ct value is >30. In that case, microarray analysis may not be performed. Alternately, microarray results may be attenuated, e.g., given a lower priority as compared to the results of other molecular profiling techniques.

In some embodiments, molecular profiling is always performed on certain genes or gene products, whereas the profiling of other genes or gene products is optional. For example, IHC expression profiling may be performed on at least SPARC, TOP2A and/or PTEN. Similarly, microarray expression profiling may be performed on at least CD52. In other embodiments, genes in addition to those listed above are used to identify a treatment. For example, the group of genes used for the IHC expression profiling can further comprise DCK, EGFR, BRCA1, CK 14, CK 17, CK 5/6, E-Cadherin, p95, PARP-1, SPARC and TLE3. In some embodiments, the group of genes used for the IHC expression profiling further comprises Cox-2 and/or Ki-67. In some embodiments, HSPCA is assayed by microarray analysis. In some embodiments, FISH mutation is performed on c-Myc and TOP2A. In some embodiments, sequencing is performed on PI3K.

The methods of the invention can be used in any setting wherein differential expression or mutation analysis have been linked to efficacy of various treatments. In some embodiments, the methods are used to identify candidate treatments for a subject having a cancer. Under these conditions, the sample used for molecular profiling preferably comprises cancer cells. The percentage of cancer in a sample can be determined by methods known to those of skill in the art, e.g., using pathology techniques. Cancer cells can also be enriched from a sample, e.g., using microdissection techniques or the like. A sample may be required to have a certain threshold of cancer cells before it is used for molecular profiling. The threshold can be at least about 5, 10, 20, 30, 40, 50, 60, 70, 80, 90 or 95% cancer cells. The threshold can depend on the analysis method. For example, a technique that reveals expression in individual cells may require a lower threshold that a technique that used a sample extracted from a mixture of different cells. In some embodiments, the diseased sample is compared to a normal sample taken from the same patient, e.g., adjacent but non-cancer tissue.

In embodiments, the methods of the invention are used detect gene fusions, such as those listed in U.S. patent application Ser. No. 12/658,770, filed Feb. 12, 2010; International PCT Patent Application PCT/US2010/000407, filed Feb. 11, 2010; and International PCT Patent Application PCT/US2010/54366, filed Oct. 27, 2010; all of which applications are incorporated by reference herein in their entirety. A fusion gene is a hybrid gene created by the juxtaposition of two previously separate genes. This can occur by chromosomal translocation or inversion, deletion or via trans-splicing. The resulting fusion gene can cause abnormal temporal and spatial expression of genes, leading to abnormal expression of cell growth factors, angiogenesis factors, tumor promoters or other factors contributing to the neoplastic transformation of the cell and the creation of a tumor. For example, such fusion genes can be oncogenic due to the juxtaposition of: 1) a strong promoter region of one gene next to the coding region of a cell growth factor, tumor promoter or other gene promoting oncogenesis leading to elevated gene expression, or 2) due to the fusion of coding regions of two different genes, giving rise to a chimeric gene and thus a chimeric protein with abnormal activity. Fusion genes are characteristic of many cancers. Once a therapeutic intervention is associated with a fusion, the presence of that fusion in any type of cancer identifies the therapeutic intervention as a candidate therapy for treating the cancer.

The presence of fusion genes, e.g., those described in U.S. patent application Ser. No. 12/658,770, filed Feb. 12, 2010; International PCT Patent Application PCT/US2010/000407, filed Feb. 11, 2010; and International PCT Patent Application PCT/US2010/54366, filed Oct. 27, 2010 or elsewhere herein, can be used to guide therapeutic selection. For example, the BCR-ABL gene fusion is a characteristic molecular aberration in ˜90% of chronic myelogenous leukemia (CML) and in a subset of acute leukemias (Kurzrock et al., Annals of Internal Medicine 2003; 138:819-830). The BCR-ABL results from a translocation between chromosomes 9 and 22, commonly referred to as the Philadelphia chromosome or Philadelphia translocation. The translocation brings together the 5′ region of the BCR gene and the 3′ region of ABL1, generating a chimeric BCR-ABL1 gene, which encodes a protein with constitutively active tyrosine kinase activity (Mittleman et al., Nature Reviews Cancer 2007; 7:233-245). The aberrant tyrosine kinase activity leads to de-regulated cell signaling, cell growth and cell survival, apoptosis resistance and growth factor independence, all of which contribute to the pathophysiology of leukemia (Kurzrock et al., Annals of Internal Medicine 2003; 138:819-830). Patients with the Philadelphia chromosome are treated with imatinib and other targeted therapies. Imatinib binds to the site of the constitutive tyrosine kinase activity of the fusion protein and prevents its activity. Imatinib treatment has led to molecular responses (disappearance of BCR-ABL+ blood cells) and improved progression-free survival in BCR-ABL+ CML patients (Kantarjian et al., Clinical Cancer Research 2007; 13:1089-1097).

Another fusion gene, IGH-MYC, is a defining feature of ˜80% of Burkitt's lymphoma (Ferry et al. Oncologist 2006; 11:375-83). The causal event for this is a translocation between chromosomes 8 and 14, bringing the c-Myc oncogene adjacent to the strong promoter of the immunoglobulin heavy chain gene, causing c-myc overexpression (Mittleman et al., Nature Reviews Cancer 2007; 7:233-245). The c-myc rearrangement is a pivotal event in lymphomagenesis as it results in a perpetually proliferative state. It has wide ranging effects on progression through the cell cycle, cellular differentiation, apoptosis, and cell adhesion (Ferry et al. Oncologist 2006; 11:375-83).

A number of recurrent fusion genes have been catalogued in the Mittleman database (cgap.nci.nih.gov/Chromosomes/Mitelman). The gene fusions can be used to characterize neoplasms and cancers and guide therapy using the subject methods described herein. For example, TMPRSS2-ERG, TMPRSS2-ETV and SLC45A3-ELK4 fusions can be detected to characterize prostate cancer; and ETV6-NTRK3 and ODZ4-NRG1 can be used to characterize breast cancer. The EML4-ALK, RLF-MYCL1, TGF-ALK, or CD74-ROS1 fusions can be used to characterize a lung cancer. The ACSL3-ETV1, C150RF21-ETV1, F1135294-ETV1, HERV-ETV1, TMPRSS2-ERG, TMPRSS2-ETV1/4/5, TMPRSS2-ETV4/5, SLC5A3-ERG, SLC5A3-ETV1, SLC5A3-ETV5 or KLK2-ETV4 fusions can be used to characterize a prostate cancer. The GOPC-ROS1 fusion can be used to characterize a brain cancer. The CHCHD7-PLAG1, CTNNB1-PLAG1, FHIT-HMGA2, HMGA2-NFIB, LIFR-PLAG1, or TCEA1-PLAG1 fusions can be used to characterize a head and neck cancer. The ALPHA-TFEB, NONO-TFE3, PRCC-TFE3, SFPQ-TFE3, CLTC-TFE3, or MALAT1-TFEB fusions can be used to characterize a renal cell carcinoma (RCC). The AKAP9-BRAF, CCDC6-RET, ERC1-RETM, GOLGAS-RET, HOOK3-RET, HRH4-RET, KTN1-RET, NCOA4-RET, PCM1-RET, PRKARA1A-RET, RFG-RET, RFG9-RET, Ria-RET, TGF-NTRK1, TPM3-NTRK1, TPM3-TPR, TPR-MET, TPR-NTRK1, TRIM24-RET, TRIM27-RET or TRIM33-RET fusions can be used to characterize a thyroid cancer and/or papillary thyroid carcinoma; and the PAX8-PPARy fusion can be analyzed to characterize a follicular thyroid cancer. Fusions that are associated with hematological malignancies include without limitation TTL-ETV6, CDK6-MLL, CDK6-TLX3, ETV6-FLT3, ETV6-RUNX1, ETV6-TTL, MLL-AFF1, MLL-AFF3, MLL-AFF4, MLL-GAS7, TCBA1-ETV6, TCF3-PBX1 or TCF3-TFPT, which are characteristic of acute lymphocytic leukemia (ALL); BCL11B-TLX3, IL2-TNFRFS17, NUP214-ABL1, NUP98-CCDC28A, TAL1-STIL, or ETV6-ABL2, which are characteristic of T-cell acute lymphocytic leukemia (T-ALL); ATIC-ALK, KIAA1618-ALK, MSN-ALK, MYH9-ALK, NPM1-ALK, TGF-ALK or TPM3-ALK, which are characteristic of anaplastic large cell lymphoma (ALCL); BCR-ABL1, BCR-JAK2, ETV6-EVI1, ETV6-MN1 or ETV6-TCBA1, characteristic of chronic myelogenous leukemia (CML); CBFB-MYH11, CHIC2-ETV6, ETV6-ABL1, ETV6-ABL2, ETV6-ARNT, ETV6-CDX2, ETV6-HLXB9, ETV6-PER1, MEF2D-DAZAP1, AML-AFF1, MLL-ARHGAP26, MLL-ARHGEF12, MLL-CASC5, MLL-CBL,MLL-CREBBP, MLL-DAB21P, MLL-ELL, MLL-EP300, MLL-EPS15, MLL-FNBP1, MLL-FOXO3A, MLL-GMPS, MLL-GPHN, MLL-MLLT1, MLL-MLLT11, MLL-MLLT3, MLL-MLLT6, MLL-MYO1F, MLL-PICALM, MLL-SEPT2, MLL-SEPT6, MLL-SORBS2, MYST3-SORBS2, MYST-CREBBP, NPM1-MLF1, NUP98-HOXA13, PRDM16-EVI1, RABEP1-PDGFRB, RUNX1-EVI1, RUNX1-MDS1, RUNX1-RPL22, RUNX1-RUNXIT1, RUNX1-SH3D19, RUNX1-USP42, RUNX1-YTHDF2, RUNX1-ZNF687, or TAF15-ZNF-384, which are characteristic of acute myeloid leukemia (AML); CCND1-FSTL3, which is characteristic of chronic lymphocytic leukemia (CLL); BCL3-MYC, MYC-BTG1, BCL7A-MYC, BRWD3-ARHGAP20 or BTG1-MYC, which are characteristic of B-cell chronic lymphocytic leukemia (B-CLL); CITTA-BCL6, CLTC-ALK, IL21R-BCL6, PIM1-BCL6, TFCR-BCL6, IKZF1-BCL6 or SEC31A-ALK, which are characteristic of diffuse large B-cell lymphomas (DLBCL); FLIP1-PDGFRA, FLT3-ETV6, KIAA1509-PDGFRA, PDE4DIP-PDGFRB, NIN-PDGFRB, TP53BP1-PDGFRB, or TPM3-PDGFRB, which are characteristic of hyper eosinophilia/chronic eosinophilia; and IGH-MYC or LCP1-BCL6, which are characteristic of Burkitt's lymphoma. One of skill will understand that additional fusions, including those yet to be identified to date, can be used to guide treatment once their presence is associated with a therapeutic intervention.

The fusion genes and gene products can be detected using one or more techniques described herein. In some embodiments, the sequence of the gene or corresponding mRNA is determined, e.g., using Sanger sequencing, NextGen sequencing, pyrosequencing, DNA microarrays, etc. Chromosomal abnormalities can be assessed using FISH or PCR techniques, among others. For example, a break apart probe can be used for FISH detection of ALK fusions such as EML4-ALK, KIF5B-ALK and/or TFG-ALK. As an alternate, PCR can be used to amplify the fusion product, wherein amplification or lack thereof indicates the presence or absence of the fusion, respectively. In some embodiments, the fusion protein fusion is detected. Appropriate methods for protein analysis include without limitation mass spectroscopy, electrophoresis (e.g., 2D gel electrophoresis or SDS-PAGE) or antibody related techniques, including immunoassay, protein array or immunohistochemistry. The techniques can be combined. As a non-limiting example, indication of an ALK fusion by FISH can be confirmed for ALK expression using IHC, or vice versa.

### Treatment Selection

The systems and methods allow identification of one or more therapeutic targets whose projected efficacy can be linked to therapeutic efficacy, ultimately based on the molecular profiling. Illustrative schemes for using molecular profiling to identify a treatment regime are shown in FIGS. 2, 49A-B and 50, each of which is described in further detail herein. The invention comprises use of molecular profiling results to suggest associations with treatment responses. In an embodiment, the appropriate biomarkers for molecular profiling are selected on the basis of the subject's tumor type. These suggested biomarkers can be used to modify a default list of biomarkers. In other embodiments, the molecular profiling is independent of the source material. In some embodiments, rules are used to provide the suggested chemotherapy treatments based on the molecular profiling test results. In an embodiment, the rules are generated from abstracts of the peer reviewed clinical oncology literature. Expert opinion rules can be used but are optional. In an embodiment, clinical citations are assessed for their relevance to the methods of the invention using a hierarchy derived from the evidence grading system used by the United States Preventive Services Taskforce. The “best evidence” can be used as the basis for a rule. The simplest rules are constructed in the format of “if biomarker positive then treatment option one, else treatment option two.” Treatment options comprise no treatment with a specific drug, treatment with a specific drug or treatment with a combination of drugs. In some embodiments, more complex rules are constructed that involve the interaction of two or more biomarkers. In such cases, the more complex interactions are typically supported by clinical studies that analyze the interaction between the biomarkers included in the rule. Finally, a report can be generated that describes the association of the chemotherapy response and the biomarker and a summary statement of the best evidence supporting the treatments selected. Ultimately, the treating physician will decide on the best course of treatment.

As a non-limiting example, molecular profiling might reveal that the EGFR gene is amplified or overexpressed, thus indicating selection of a treatment that can block EGFR activity, such as the monoclonal antibody inhibitors cetuximab and panitumumab, or small molecule kinase inhibitors effective in patients with activating mutations in EGFR such as gefitinib, erlotinib, and lapatinib. Other anti-EGFR monoclonal antibodies in clinical development include zalutumumab, nimotuzumab, and matuzumab. The candidate treatment selected can depend on the setting revealed by molecular profiling. For example, kinase inhibitors are often prescribed with EGFR is found to have activating mutations. Continuing with the illustrative embodiment, molecular profiling may also reveal that some or all of these treatments are likely to be less effective. For example, patients taking gefitinib or erlotinib eventually develop drug resistance mutations in EGFR. Accordingly, the presence of a drug resistance mutation would contraindicate selection of the small molecule kinase inhibitors. One of skill will appreciate that this example can be expanded to guide the selection of other candidate treatments that act against genes or gene products whose differential expression is revealed by molecular profiling. Similarly, candidate agents known to be effective against diseased cells carrying certain nucleic acid variants can be selected if molecular profiling reveals such variants.

As another example, consider the drug imatinib, currently marketed by Novartis as Gleevec in the US in the form of imatinib mesylate. Imatinib is a 2-phenylaminopyrimidine derivative that functions as a specific inhibitor of a number of tyrosine kinase enzymes. It occupies the tyrosine kinase active site, leading to a decrease in kinase activity. Imatinib has been shown to block the activity of Abelson cytoplasmic tyrosine kinase (ABL), c-Kit and the platelet-derived growth factor receptor (PDGFR). Thus, imatinib can be indicated as a candidate therapeutic for a cancer determined by molecular profiling to overexpress ABL, c-KIT or PDGFR. Imatinib can be indicated as a candidate therapeutic for a cancer determined by molecular profiling to have mutations in ABL, c-KIT or PDGFR that alter their activity, e.g., constitutive kinase activity of ABLs caused by the BCR-ABL mutation. As an inhibitor of PDGFR, imatinib mesylate appears to have utility in the treatment of a variety of dermatological diseases.

Cancer therapies that can be identified as candidate treatments by the methods of the invention include without limitation: 13-cis-Retinoic Acid, 2-CdA, 2-Chlorodeoxyadenosine, 5-Azacitidine, 5-Fluorouracil, 5-FU, 6-Mercaptopurine, 6-MP, 6-TG, 6-Thioguanine, Abraxane, Accutane®, Actinomycin-D, Adriamycin®, Adrucil®, Afinitor®, Agrylin®, Ala-Cort®, Aldesleukin, Alemtuzumab, ALIMTA, Alitretinoin, Alkaban-AQ®, Alkeran®, All-transretinoic Acid, Alpha Interferon, Altretamine, Amethopterin, Amifostine, Aminoglutethimide, Anagrelide, Anandron®, Anastrozole, Arabinosylcytosine, Ara-C, Aranesp®, Aredia®, Arimidex®, Aromasin®, Arranon®, Arsenic Trioxide, Asparaginase, ATRA, Avastin®, Azacitidine, BCG, BCNU, Bendamustine, Bevacizumab, Bexarotene, BEXXAR®, Bicalutamide, BiCNU, Blenoxane®, Bleomycin, Bortezomib, Busulfan, Busulfex®, C225, Calcium Leucovorin, Campath®, Camptosar®, Camptothecin-11, Capecitabine, Carac™, Carboplatin, Carmustine, Carmustine Wafer, Casodex®, CC-5013, CCI-779, CCNU, CDDP, CeeNU, Cerubidine®, Cetuximab, Chlorambucil, Cisplatin, Citrovorum Factor, Cladribine, Cortisone, Cosmegen®, CPT-11, Cyclophosphamide, Cytadren®, Cytarabine, Cytarabine Liposomal, Cytosar-U®, Cytoxan®, Dacarbazine, Dacogen, Dactinomycin, Darbepoetin Alfa, Dasatinib, Daunomycin Daunorubicin, Daunorubicin Hydrochloride, Daunorubicin Liposomal, DaunoXome®, Decadron, Decitabine, Delta-Cortef®, Deltasone®, Denileukin, Diftitox, DepoCyt™, Dexamethasone, Dexamethasone Acetate Dexamethasone Sodium Phosphate, Dexasone, Dexrazoxane, DHAD, DIC, Diodex Docetaxel, Doxil®, Doxorubicin, Doxorubicin Liposomal, Droxia™, DTIC, DTIC-Dome®, Duralone®, Efudex®, Eligard™ Ellence™, Eloxatin™, Elspar®, Emcyt®, Epirubicin, Epoetin Alfa, Erbitux, Erlotinib, Erwinia L-asparaginase, Estramustine, Ethyol Etopophos®, Etoposide, Etoposide Phosphate, Eulexin®, Everolimus, Evista®, Exemestane, Fareston®, Faslodex®, Femara®, Filgrastim, Floxuridine, Fludara®, Fludarabine, Fluoroplex®, Fluorouracil, Fluorouracil (cream), Fluoxymesterone, Flutamide, Folinic Acid, FUDR®, Fulvestrant, G-CSF, Gefitinib, Gemcitabine, Gemtuzumab ozogamicin, Gemzar, Gleevec™, Gliadel® Wafer, GM-CSF, Goserelin, Granulocyte—Colony Stimulating Factor, Granulocyte Macrophage Colony Stimulating Factor, Halotestin®, Herceptin®, Hexadrol, Hexalen®, Hexamethylmelamine, HMM, Hycamtin®, Hydrea®, Hydrocort Acetate®, Hydrocortisone, Hydrocortisone Sodium Phosphate, Hydrocortisone Sodium Succinate, Hydrocortone Phosphate, Hydroxyurea, Ibritumomab, Ibritumomab, Tiuxetan, Idamycin®, Idarubicin, Ifex®, IFN-alpha, Ifosfamide, IL-11, IL-2, Imatinib mesylate, Imidazole Carboxamide, Interferon alfa, Interferon Alfa-2b (PEG Conjugate), Interleukin-2, Interleukin-11, Intron A® (interferon alfa-2b), Iressa®, Irinotecan, Isotretinoin, Ixabepilone, Ixempra™, Kidrolase (t), Lanacort®, Lapatinib, L-asparaginase, LCR, Lenalidomide, Letrozole, Leucovorin, Leukeran, Leukine™, Leuprolide, Leurocristine, Leustatin™, Liposomal Ara-C Liquid Fred®, Lomustine, L-PAM, L-Sarcolysin, Lupron®, Lupron Depot®, Matulane®, Maxidex, Mechlorethamine, Mechlorethamine Hydrochloride, Medralone®, Medrol®, Megace®, Megestrol, Megestrol Acetate, Melphalan, Mercaptopurine, Mesna, Mesnex™, Methotrexate, Methotrexate Sodium, Methylprednisolone, Meticorten®, Mitomycin, Mitomycin-C, Mitoxantrone, M-Prednisol®, MTC, MTX, Mustargen®, Mustine, Mutamycin®, Myleran®, Mylocel™, Mylotarg®, Navelbine®, Nelarabine, Neosar®, Neulasta™, Neumega®, Neupogen®, Nexavar®, Nilandron®, Nilutamide, Nipent®, Nitrogen Mustard, Novaldex®, Novantrone®, Octreotide, Octreotide acetate, Oncospar®, Oncovin®, Ontak®, Onxal™ Oprevelkin, Orapred®, Orasone®, Oxaliplatin, Paclitaxel, Paclitaxel Protein-bound, Pamidronate, Panitumumab, Panretin®, Paraplatin®, Pediapred®, PEG Interferon, Pegaspargase, Pegfilgrastim, PEG-INTRON™, PEG-L-asparaginase, PEMETREXED, Pentostatin, Phenylalanine Mustard, Platinol®, Platinol-AQ®, Prednisolone, Prednisone, Prelone®, Procarbazine, PROCRIT®, Proleukin®, Prolifeprospan 20 with Carmustine Implant, Purinethol®, Raloxifene, Revlimid®, Rheumatrex®, Rituxan®, Rituximab, Roferon-A® (Interferon Alfa-2a), Rubex®, Rubidomycin hydrochloride, Sandostatin®, Sandostatin LAR®, Sargramostim, Solu-Cortef®, Solu-Medrol®, Sorafenib, SPRYCEL™, STI-571, Streptozocin, SU11248, Sunitinib, Sutent®, Tamoxifen, Tarceva®, Targretin®, Taxol®, Taxotere®, Temodar®, Temozolomide, Temsirolimus, Teniposide, TESPA, Thalidomide, Thalomid®, TheraCys®, Thioguanine, Thioguanine Tabloid®, Thiophosphoamide, Thioplex®, Thiotepa, TICE®, Toposar®, Topotecan, Toremifene, Torisel®, Tositumomab, Trastuzumab, Treanda®, Tretinoin, Trexall™, Trisenox®, TSPA, TYKERB®, VCR, Vectibix™, Velban®, Velcade®, VePesid®, Vesanoid®, Viadur™, Vidaza®, Vinblastine, Vinblastine Sulfate, Vincasar Pfs®, Vincristine, Vinorelbine, Vinorelbine tartrate, VLB, VM-26, Vorinostat, VP-16, Vumon®, Xeloda®, Zanosar®, Zevalin™, Zinecard®, Zoladex®, Zoledronic acid, Zolinza, Zometa®, and any appropriate combinations thereof.

The candidate treatments identified according to the subject methods can be chosen from the class of therapeutic agents identified as Anthracyclines and related substances, Anti-androgens, Anti-estrogens, Antigrowth hormones (e.g., Somatostatin analogs), Combination therapy (e.g., vincristine, bcnu, melphalan, cyclophosphamide, prednisone (VBMCP)), DNA methyltransferase inhibitors, Endocrine therapy—Enzyme inhibitor, Endocrine therapy—other hormone antagonists and related agents, Folic acid analogs (e.g., methotrexate), Folic acid analogs (e.g., pemetrexed), Gonadotropin releasing hormone analogs, Gonadotropin-releasing hormones, Monoclonal antibodies (EGFR-Targeted—e.g., panitumumab, cetuximab), Monoclonal antibodies (Her2-Targeted—e.g., trastuzumab), Monoclonal antibodies (Multi-Targeted—e.g., alemtuzumab), Other alkylating agents, Other antineoplastic agents (e.g., asparaginase), Other antineoplastic agents (e.g., ATRA), Other antineoplastic agents (e.g., bexarotene), Other antineoplastic agents (e.g., celecoxib), Other antineoplastic agents (e.g., gemcitabine), Other antineoplastic agents (e.g., hydroxyurea), Other antineoplastic agents (e.g., irinotecan, topotecan), Other antineoplastic agents (e.g., pentostatin), Other cytotoxic antibiotics, Platinum compounds, Podophyllotoxin derivatives (e.g., etoposide), Progestogens, Protein kinase inhibitors (EGFR-Targeted), Protein kinase inhibitors (Her2 targeted therapy—e.g., lapatinib), Pyrimidine analogs (e.g., cytarabine), Pyrimidine analogs (e.g., fluoropyrimidines), Salicylic acid and derivatives (e.g., aspirin), Src-family protein tyrosine kinase inhibitors (e.g., dasatinib), Taxanes, Taxanes (e.g., nab-paclitaxel), Vinca Alkaloids and analogs, Vitamin D and analogs, Monoclonal antibodies (Multi-Targeted—e.g., bevacizumab), Protein kinase inhibitors (e.g., imatinib, sorafenib, sunitinib), Tyrosine Kinase inhibitors (TKI) (e.g., vemurafenib, sorafenib, imatinib, sunitinib, erlotinib, gefitinib, crizotinib, lapatinib).

In some embodiments, the candidate treatments identified according to the subject methods are chosen from at least the groups of treatments consisting of 5-fluorouracil, abarelix, alemtuzumab, aminoglutethimide, anastrozole, asparaginase, aspirin, ATRA, azacitidine, bevacizumab, bexarotene, bicalutamide, calcitriol, capecitabine, carboplatin, celecoxib, cetuximab, chemotherapy, cholecalciferol, cisplatin, cytarabine, dasatinib, daunorubicin, decitabine, doxorubicin, epirubicin, erlotinib, etoposide, exemestane, flutamide, fulvestrant, gefitinib, gemcitabine, gonadorelin, goserelin, hydroxyurea, imatinib, irinotecan, lapatinib, letrozole, leuprolide, liposomal-doxorubicin, medroxyprogesterone, megestrol, megestrol acetate, methotrexate, mitomycin, nab-paclitaxel, octreotide, oxaliplatin, paclitaxel, panitumumab, pegaspargase, pemetrexed, pentostatin, sorafenib, sunitinib, tamoxifen, Taxanes, temozolomide, toremifene, trastuzumab, VBMCP, and vincristine. The candidate treatments can be any of those in Tables 3-5, 7-22, 28, 29, 33, 36 or 37 herein.

### Rules Engine

In some embodiments, a database is created that maps treatments and molecular profiling results. The treatment information can include the projected efficacy of a therapeutic agent against cells having certain attributes that can be measured by molecular profiling. The molecular profiling can include differential expression or mutations in certain genes, proteins, or other biological molecules of interest. Through the mapping, the results of the molecular profiling can be compared against the database to select treatments. The database can include both positive and negative mappings between treatments and molecular profiling results. In some embodiments, the mapping is created by reviewing the literature for links between biological agents and therapeutic agents. For example, a journal article, patent publication or patent application publication, scientific presentation, etc can be reviewed for potential mappings. The mapping can include results of in vivo, e.g., animal studies or clinical trials, or in vitro experiments, e.g., cell culture. Any mappings that are found can be entered into the database, e.g., cytotoxic effects of a therapeutic agent against cells expressing a gene or protein. In this manner, the database can be continuously updated. It will be appreciated that the methods of the invention are updated as well.

The rules can be generated by evidence-based literature review. Biomarker research continues to provide a better understanding of the clinical behavior and biology of cancer. This body of literature can be maintained in an up-to-date data repository incorporating recent clinical studies relevant to treatment options and potential clinical outcomes. The studies can be ranked so that only those with the strongest or most reliable evidence are selected for rules generation. For example, the rules generation can employ the grading system from the current methods of the U.S. Preventive Services Task Force. The literature evidence can be reviewed and evaluated based on the strength of clinical evidence supporting associations between biomarkers and treatments in the literature study. This process can be performed by a staff of scientists, physicians and other skilled reviewers. The process can also be automated in whole or in part by using language search and heuristics to identify relevant literature. The rules can be generated by a review of a plurality of literature references, e.g., tens, hundreds, thousands or more literature articles.

In another aspect, the invention provides a method of generating a set of evidence-based associations, comprising: (a) searching one or more literature database by a computer using an evidence-based medicine search filter to identify articles comprising a gene or gene product thereof, a disease, and one or more therapeutic agent; (b) filtering the articles identified in (a) to compile evidence-based associations comprising the expected benefit and/or the expected lack of benefit of the one or more therapeutic agent for treating the disease given the status of the gene or gene product; (c) adding the evidence-based associations compiled in (b) to the set of evidence-based associations; and (d) repeating steps (a)-(c) for an additional gene or gene product thereof. The status of the gene can include one or more assessments as described herein which relate to a biological state, e.g., one or more of an expression level, a copy number, and a mutation. The genes or gene products thereof can be one or more genes or gene products thereof selected from Table 2, Table 6 or Table 25. For example, the method can be repeated for 1, 2, 3, 4, 5, 6, 7, 8, 9, 10, 15, 20, 25, 30, 40, 50 or more of the genes or gene products thereof in Table 2, Table 6 or Table 25. The disease can be a disease described here, e.g., in embodiment the disease comprises a cancer. The one or more literature database can be selected from the group consisting of the National Library of Medicine's (NLM's) MEDLINE™ database of citations, a patent literature database, and a combination thereof.

Evidence-based medicine (EBM) or evidence-based practice (EBP) aims to apply the best available evidence gained from the scientific method to clinical decision making. This approach assesses the strength of evidence of the risks and benefits of treatments (including lack of treatment) and diagnostic tests. Evidence quality can be assessed based on the source type (from meta-analyses and systematic reviews of double-blind, placebo-controlled clinical trials at the top end, down to conventional wisdom at the bottom), as well as other factors including statistical validity, clinical relevance, currency, and peer-review acceptance. Evidence-based medicine filters are searches that have been developed to facilitate searches in specific areas of clinical medicine related to evidence-based medicine (diagnosis, etiology, meta-analysis, prognosis and therapy). They are designed to retrieve high quality evidence from published studies appropriate to decision-making. The evidence-based medicine filter used in the invention can be selected from the group consisting of a generic evidence-based medicine filter, a McMaster University optimal search strategy evidence-based medicine filter, a University of York statistically developed search evidence-based medicine filter, and a University of California San Francisco systemic review evidence-based medicine filter. See e.g., US Patent Publication 20080215570; Shojania and Bero. Taking advantage of the explosion of systematic reviews: an efficient MEDLINE search strategy. Eff Clin Pract. 2001 July-August; 4(4):157-62; Ingui and Rogers. Searching for clinical prediction rules in MEDLINE. J Am Med Inform Assoc. 2001 July-August; 8(4):391-7; Haynes et al., Optimal search strategies for retrieving scientifically strong studies of treatment from Medline: analytical survey. BMJ. 2005 May 21; 330(7501):1179; Wilczynski and Haynes. Consistency and accuracy of indexing systematic review articles and meta-analyses in medline. Health Info Libr J. 2009 September; 26(3):203-10; which references are incorporated by reference herein in their entirety. A generic filter can be a customized filter based on an algorithm to identify the desired references from the one or more literature database. For example, the method can use one or more approach as described in U.S. Pat. No. 5,168,533 to Kato et al., U.S. Pat. No. 6,886,010 to Kostoff, or US Patent Application Publication No. 20040064438 to Kostoff; which references are incorporated by reference herein in their entirety.

The further filtering of articles identified by the evidence-based medicine filter can be performed using a computer, by one or more expert user, or combination thereof. The one or more expert can be a trained scientist or physician. In embodiments, the set of evidence-based associations comprise one or more of the rules in any of Tables 3-4, 7-25 or 27. For example, the set of evidence-based associations can include at least 5, 10, 25, 50 or 100 rules in Tables 3-4, 7-25 or 27. In some embodiments, the set of evidence-based associations comprises or consists of all of the rules in any of Tables 3-4, 7-25 or 27. In an aspect, the invention provides a computer readable medium comprising the set of evidence-based associations generated by the subject methods. The invention further provides a computer readable medium comprising one or more rules in any of Tables 3-4, 7-25 or 27 herein. In an embodiment, the computer readable medium comprises at least 5, 10, 25, 50 or 100 rules in any of Tables 3-4, 7-25 or 27. For example, the computer readable medium can comprise all rules in any of Tables 3-4, 7-25 or 27, e.g., all rules in Tables 3-4, 7-25 or 27.

The rules for the mappings can contain a variety of supplemental information. In some embodiments, the database contains prioritization criteria. For example, a treatment with more projected efficacy in a given setting can be preferred over a treatment projected to have lesser efficacy. A mapping derived from a certain setting, e.g., a clinical trial, may be prioritized over a mapping derived from another setting, e.g., cell culture experiments. A treatment with strong literature support may be prioritized over a treatment supported by more preliminary results. A treatment generally applied to the type of disease in question, e.g., cancer of a certain tissue origin, may be prioritized over a treatment that is not indicated for that particular disease. Mappings can include both positive and negative correlations between a treatment and a molecular profiling result. In a non-limiting example, one mapping might suggest use of a kinase inhibitor like erlotinib against a tumor having an activating mutation in EGFR, whereas another mapping might suggest against that treatment if the EGFR also has a drug resistance mutation. Similarly, a treatment might be indicated as effective in cells that overexpress a certain gene or protein but indicated as not effective if the gene or protein is underexpressed.

The selection of a candidate treatment for an individual can be based on molecular profiling results from any one or more of the methods described. Alternatively, selection of a candidate treatment for an individual can be based on molecular profiling results from more than one of the methods described. For example, selection of treatment for an individual can be based on molecular profiling results from FISH alone, IHC alone, or microarray analysis alone. In other embodiments, selection of treatment for an individual can be based on molecular profiling results from IHC, FISH, and microarray analysis; IHC and FISH; IHC and microarray analysis, or FISH and microarray analysis. Selection of treatment for an individual can also be based on molecular profiling results from sequencing or other methods of mutation detection. Molecular profiling results may include mutation analysis along with one or more methods, such as IHC, immunoassay, and/or microarray analysis. Different combinations and sequential results can be used. For example, treatment can be prioritized according the results obtained by molecular profiling. In an embodiment, the prioritization is based on the following algorithm: 1) IHC/FISH and microarray indicates same target as a first priority; 2) IHC positive result alone next priority; or 3) microarray positive result alone as last priority. Sequencing can also be used to guide selection. In some embodiments, sequencing reveals a drug resistance mutation so that the effected drug is not selected even if techniques including IHC, microarray and/or FISH indicate differential expression of the target molecule. Any such contraindication, e.g., differential expression or mutation of another gene or gene product may override selection of a treatment.

An illustrative listing of microarray expression results versus predicted treatments is presented in Table 3. As disclosed herein, molecular profiling is performed to determine whether a gene or gene product is differentially expressed in a sample as compared to a control. The expression status of the gene or gene product is used to select agents that are predicted to be efficacious or not. For example, Table 3 shows that overexpression of the ADA gene or protein points to pentostatin as a possible treatment. On the other hand, underexpression of the ADA gene or protein implicates resistance to cytarabine, suggesting that cytarabine is not an optimal treatment.

TABLE 3
Molecular Profiling Results and Predicted Treatments

| Gene Name | Expression Status | Candidate Agent(s) | Possible Resistance |
|---|---|---|---|
| ADA | Overexpressed | pentostatin | |
| ADA | Underexpressed | | cytarabine |
| AR | Overexpressed | abarelix, bicalutamide, | |
| | | flutamide, gonadorelin, | |
| | | goserelin, leuprolide | |
| ASNS | Underexpressed | asparaginase, | |
| | | pegaspargase | |
| BCRP (ABCG2) | Overexpressed | | cisplatin, carboplatin, |
| | | | irinotecan, topotecan |
| BRCA1 | Underexpressed | mitomycin | |
| BRCA2 | Underexpressed | mitomycin | |
| CD52 | Overexpressed | alemtuzumab | |
| CDA | Overexpressed | | cytarabine |
| CES2 | Overexpressed | irinotecan | |
| c-kit | Overexpressed | sorafenib, sunitinib, | |
| | | imatinib | |
| COX-2 | Overexpressed | celecoxib | |
| DCK | Overexpressed | gemcitabine | cytarabine |
| DHFR | Underexpressed | methotrexate, | |
| | | pemetrexed | |
| DHFR | Overexpressed | | methotrexate |
| DNMT1 | Overexpressed | azacitidine, decitabine | |
| DNMT3A | Overexpressed | azacitidine, decitabine | |
| DNMT3B | Overexpressed | azacitidine, decitabine | |
| EGFR | Overexpressed | erlotinib, gefitinib, | |
| | | cetuximab, panitumumab | |
| EML4-ALK | Overexpressed (present) | crizotinib | |
| EPHA2 | Overexpressed | dasatinib | |
| ER | Overexpressed | anastrazole, exemestane, | |
| | | fulvestrant, letrozole, | |
| | | megestrol, tamoxifen, | |
| | | medroxyprogesterone, | |
| | | toremifene, | |
| | | aminoglutethimide | |
| ERCC1 | Overexpressed | | carboplatin, cisplatin |
| GART | Underexpressed | pemetrexed | |
| HER-2 (ERBB2) | Overexpressed | trastuzumab, lapatinib | |
| HIF-1α | Overexpressed | sorafenib, sunitinib, | |
| | | bevacizumab | |
| IκB-α | Overexpressed | bortezomib | |
| MGMT | Underexpressed | temozolomide | |
| MGMT | Overexpressed | | temozolomide |
| MRP1 (ABCC1) | Overexpressed | | etoposide, paclitaxel, |
| | | | docetaxel, |
| | | | vinblastine, |
| | | | vinorelbine, |
| | | | topotecan, teniposide |
| P-gp (ABCB1) | Overexpressed | | doxorubicin, |
| | | | etoposide, epirubicin, |
| | | | paclitaxel, docetaxel, |
| | | | vinblastine, |
| | | | vinorelbine, |
| | | | topotecan, teniposide, |
| | | | liposomal |
| | | | doxorubicin |
| PDGFR-α | Overexpressed | sorafenib, sunitinib, | |
| | | imatinib | |
| PDGFR-β | Overexpressed | sorafenib, sunitinib, | |
| | | imatinib | |
| PR | Overexpressed | exemestane, fulvestrant, | |
| | | gonadorelin, goserelin, | |
| | | medroxyprogesterone, | |
| | | megestrol, tamoxifen, | |
| | | toremifene | |
| RARA | Overexpressed | ATRA | |
| RRM1 | Underexpressed | gemcitabine, | |
| | | hydroxyurea | |
| RRM2 | Underexpressed | gemcitabine, | |
| | | hydroxyurea | |
| RRM2B | Underexpressed | gemcitabine, | |
| | | hydroxyurea | |
| RXR-α | Overexpressed | bexarotene | |
| RXR-β | Overexpressed | bexarotene | |
| SPARC | Overexpressed | nab-paclitaxel | |
| SRC | Overexpressed | dasatinib | |
| SSTR2 | Overexpressed | octreotide | |
| SSTR5 | Overexpressed | octreotide | |
| TOPO I | Overexpressed | irinotecan, topotecan | |
| TOPO IIα | Overexpressed | doxorubicin, epirubicin, | |
| | | liposomal- doxorubicin | |
| TOPO IIβ | Overexpressed | doxorubicin, epirubicin, | |
| | | liposomal- doxorubicin | |
| TS | Underexpressed | capecitabine, 5- | |
| | | fluorouracil, pemetrexed | |
| TS | Overexpressed | | capecitabine, 5- |
| | | | fluorouracil |
| VDR | Overexpressed | calcitriol, cholecalciferol | |
| VEGFR1 (Flt1) | Overexpressed | sorafenib, sunitinib, | |
| | | bevacizumab | |
| VEGFR2 | Overexpressed | sorafenib, sunitinib, | |
| | | bevacizumab | |
| VHL | Underexpressed | sorafenib, sunitinib | |

Table 4 presents a selection of illustrative rules for treatment selection. The table is ordered by groups of related therapeutic agents. Each row describes a rule that maps the information derived from molecular profiling with an indication of benefit or lack of benefit for the therapeutic agent. Thus, the database contains a mapping of treatments whose biological activity is known against cancer cells that have alterations in certain genes or gene products, including gene copy alterations, chromosomal abnormalities, overexpression of or underexpression of one or more genes or gene products, or have various mutations. For each agent, a Lineage is presented as applicable which corresponds to a type of cancer associated with use of the agent. In this example, the agents can be used for all cancers. Agents with Benefit are listed along with a Benefit Summary Statement that describes molecular profiling information that relates to the predicted beneficial agent. Similarly, agents with Lack of Benefit are listed along with a Lack of Benefit Summary Statement that describes molecular profiling information that relates to the lack of benefit associated with the agent. Finally, the molecular profiling Criteria are shown. In the criteria, results from analysis using DNA microarray (DMA), IHC, FISH, and mutation analysis (MA) for one or more biomarkers is listed. For microarray analysis, expression can be reported as over (overexpressed) or under (underexpressed). When these criteria are met according to the application of the molecular profiling techniques to a sample, then the therapeutic agent or agents are predicted to have a benefit or lack of benefit as indicated in the corresponding row.

Further drug associations and rules that can be used in embodiments of the invention are found in U.S. Patent Application Publication 20100304989, filed Feb. 12, 2010; International PCT Patent Application WO/2010/093465, filed Feb. 11, 2010; and International PCT Patent Application WO/2011/056688, filed Oct. 27, 2010; all of which applications are incorporated by reference herein in their entirety. See e.g., “Table 4: Rules Summary for Treatment Selection” of WO/2011/056688.

TABLE 4
Exemplary Rules Summary for Treatment Selection

| | | | | Agents | Lack of | |
|---|---|---|---|---|---|---|
| | | Agents | Benefit | with | Benefit | |
| Therapeutic | | with | Summary | Lack of | Summary | |
| Agent | Lineage | Benefit | Statement | Benefit | Statement | Criteria |
| Protein kinase | None | sunitinib, | Presence of c- | | | DMA: VEGFR1 |
| inhibitors | | sorafenib | Kit mutation in | | | overexpressed. |
| (imatinib, | | | exon 9 has | | | DMA: HIF1A |
| sorafenib, | | | been | | | overexpressed. |
| sunitinib) | | | associated with | | | DMA: VEGFR2 |
| | | | benefit from | | | overexpressed. |
| | | | sunitinib. In | | | DMA: KIT |
| | | | addition, over | | | overexpressed. |
| | | | expression of | | | DMA: PDGFRA |
| | | | HIF1A, | | | overexpressed. |
| | | | VEGFR1, | | | DMA: PDGFRB |
| | | | VEGFR2, c- | | | overexpressed. |
| | | | Kit, PDGFRA | | | DMA: VHL |
| | | | and PDGFRB, | | | underexpressed. |
| | | | and under | | | MA: c-kit mutated - |
| | | | expression of | | | Exon 9 |
| | | | VHL have | | | |
| | | | been | | | |
| | | | associated with | | | |
| | | | benefit from | | | |
| | | | sunitinib and | | | |
| | | | sorafenib. | | | |
| Protein kinase | None | sunitinib, | Presence of c- | | | DMA: VEGFR1 |
| inhibitors | | sorafenib | Kit mutation in | | | overexpressed. |
| (imatinib, | | | exon 9 has | | | DMA: HIF1A |
| sorafenib, | | | been | | | overexpressed. |
| sunitinib) | | | associated with | | | DMA: VEGFR2 |
| | | | benefit from | | | overexpressed. |
| | | | sunitinib. In | | | DMA: KIT |
| | | | addition, over | | | overexpressed. |
| | | | expression of | | | DMA: PDGFRA |
| | | | HIF1A, | | | overexpressed. |
| | | | VEGFR1, | | | DMA: PDGFRB |
| | | | VEGFR2, c- | | | overexpressed. |
| | | | Kit, PDGFRA | | | DMA: VHL. MA: |
| | | | and PDGFRB | | | c-kit mutated - |
| | | | have been | | | Exon 9 |
| | | | associated with | | | |
| | | | benefit from | | | |
| | | | sunitinib and | | | |
| | | | sorafenib. | | | |
| Protein kinase | None | sunitinib, | Presence of c- | | | DMA: VEGFR1 |
| inhibitors | | sorafenib | Kit mutation in | | | overexpressed. |
| (imatinib, | | | exon 9 has | | | DMA: HIF1A |
| sorafenib, | | | been | | | overexpressed. |
| sunitinib) | | | associated with | | | DMA: VEGFR2. |
| | | | benefit from | | | DMA: KIT |
| | | | sunitinib. In | | | overexpressed. |
| | | | addition, over | | | DMA: PDGFRA |
| | | | expression of | | | overexpressed. |
| | | | HIF1A, | | | DMA: PDGFRB |
| | | | VEGFR1, c- | | | overexpressed. |
| | | | Kit, PDGFRA | | | DMA: VHL |
| | | | and PDGFRB, | | | underexpressed. |
| | | | and under | | | MA: c-kit mutated - |
| | | | expression of | | | Exon 9 |
| | | | VHL have | | | |
| | | | been | | | |
| | | | associated with | | | |
| | | | benefit from | | | |
| | | | sunitinib and | | | |
| | | | sorafenib. | | | |
| Protein kinase | None | sunitinib, | Presence of c- | | | DMA: VEGFR1 |
| inhibitors | | sorafenib | Kit mutation in | | | overexpressed. |
| (imatinib, | | | exon 9 has | | | DMA: HIF1A |
| sorafenib, | | | been | | | overexpressed. |
| sunitinib) | | | associated with | | | DMA: VEGFR2. |
| | | | benefit from | | | DMA: KIT |
| | | | sunitinib. In | | | overexpressed. |
| | | | addition, over | | | DMA: PDGFRA |
| | | | expression of | | | overexpressed. |
| | | | HIF1A, | | | DMA: PDGFRB |
| | | | VEGFR1, c- | | | overexpressed. |
| | | | Kit, PDGFRA | | | DMA: VHL. MA: |
| | | | and PDGFRB | | | c-kit mutated - |
| | | | have been | | | Exon 9, |
| | | | associated with | | | |
| | | | benefit from | | | |
| | | | sunitinib and | | | |
| | | | sorafenib. | | | |
| Protein kinase | None | sunitinib, | Presence of c- | | | DMA: VEGFR1. |
| inhibitors | | sorafenib | Kit mutation in | | | DMA: HIF1A |
| (imatinib, | | | exon 9 has | | | overexpressed. |
| sorafenib, | | | been | | | DMA: VEGFR2 |
| sunitinib) | | | associated with | | | overexpressed. |
| | | | benefit from | | | DMA: KIT |
| | | | sunitinib. In | | | overexpressed. |
| | | | addition, over | | | DMA: PDGFRA |
| | | | expression of | | | overexpressed. |
| | | | HIF1A, | | | DMA: PDGFRB |
| | | | VEGFR2, c- | | | overexpressed. |
| | | | Kit, PDGFRA | | | DMA: VHL |
| | | | and PDGFRB, | | | underexpressed. |
| | | | and under | | | MA: c-kit mutated - |
| | | | expression of | | | Exon 9 |
| | | | VHL have | | | |
| | | | been | | | |
| | | | associated with | | | |
| | | | benefit from | | | |
| | | | sunitinib and | | | |
| | | | sorafenib. | | | |
| Protein kinase | None | sunitinib, | Presence of c- | | | DMA: VEGFR1. |
| inhibitors | | sorafenib | Kit mutation in | | | DMA: HIF1A |
| (imatinib, | | | exon 9 has | | | overexpressed. |
| sorafenib, | | | been | | | DMA: VEGFR2 |
| sunitinib) | | | associated with | | | overexpressed. |
| | | | benefit from | | | DMA: KIT |
| | | | sunitinib. In | | | overexpressed. |
| | | | addition, over | | | DMA: PDGFRA |
| | | | expression of | | | overexpressed. |
| | | | HIF1A, | | | DMA: PDGFRB |
| | | | VEGFR2, c- | | | overexpressed. |
| | | | Kit, PDGFRA | | | DMA: VHL. MA: |
| | | | and PDGFRB | | | c-kit mutated - |
| | | | have been | | | Exon 9 |
| | | | associated with | | | |
| | | | benefit from | | | |
| | | | sunitinib and | | | |
| | | | sorafenib. | | | |
| Protein kinase | None | sunitinib, | Presence of c- | | | DMA: VEGFRI. |
| inhibitors | | sorafenib | Kit mutation in | | | DMA: HIF IA |
| (imatinib, | | | exon 9 has | | | overexpressed. |
| sorafenib, | | | been | | | DMA: VEGFR2. |
| sunitinib) | | | associated with | | | DMA: KIT |
| | | | benefit from | | | overexpressed. |
| | | | sunitinib. In | | | DMA: PDGFRA |
| | | | addition, over | | | overexpressed. |
| | | | expression of | | | DMA: PDGFRB |
| | | | HIFIA, c-Kit, | | | overexpressed. |
| | | | PDGFRA and | | | DMA: VHL |
| | | | PDGFRB, and | | | underexpressed. |
| | | | under | | | MA: c-kit mutated - |
| | | | expression of | | | Exon 9 |
| | | | VHL have | | | |
| | | | been | | | |
| | | | associated with | | | |
| | | | benefit from | | | |
| | | | sunitinib and | | | |
| | | | sorafenib. | | | |
| Protein kinase | None | sunitinib, | Presence of c- | | | DMA: VEGFRI. |
| inhibitors | | sorafenib | Kit mutation in | | | DMA: HIF IA |
| (imatinib, | | | exon 9 has | | | overexpressed. |
| sorafenib, | | | been | | | DMA: VEGFR2. |
| sunitinib) | | | associated with | | | DMA: KIT |
| | | | benefit from | | | overexpressed. |
| | | | sunitinib. In | | | DMA: PDGFRA |
| | | | addition, over | | | overexpressed. |
| | | | expression of | | | DMA: PDGFRB |
| | | | HIFIA, c-Kit, | | | overexpressed. |
| | | | PDGFRA and | | | DMA: VHL. MA: |
| | | | PDGFRB have | | | c-kit mutated - |
| | | | been | | | Exon 9 |
| | | | associated with | | | |
| | | | benefit from | | | |
| | | | sunitinib and | | | |
| | | | sorafenib. | | | |
| Protein kinase | None | sunitinib, | Presence of c- | | | DMA: VEGFRI |
| inhibitors | | sorafenib | Kit mutation in | | | overexpressed. |
| (imatinib, | | | exon 9 has | | | DMA: HIF IA |
| sorafenib, | | | been | | | overexpressed. |
| sunitinib) | | | associated with | | | DMA: VEGFR2 |
| | | | benefit from | | | overexpressed. |
| | | | sunitinib. In | | | DMA: KIT |
| | | | addition, over | | | overexpressed. |
| | | | expression of | | | DMA: PDGFRA |
| | | | HIFIA, | | | overexpressed. |
| | | | VEGFRI, | | | DMA: PDGFRB. |
| | | | VEGFR2, c- | | | DMA: VHL |
| | | | Kit and | | | underexpressed. |
| | | | PDGFRA, and | | | MA: c-kit mutated - |
| | | | under | | | Exon 9 |
| | | | expression of | | | |
| | | | VHL have | | | |
| | | | been | | | |
| | | | associated with | | | |
| | | | benefit from | | | |
| | | | sunitinib and | | | |
| | | | sorafenib. | | | |

The efficacy of various therapeutic agents given particular assay results, such as those in Table 4 above, is derived from reviewing, analyzing and rendering conclusions on empirical evidence, such as that is available the medical literature or other medical knowledge base. The results are used to guide the selection of certain therapeutic agents in a prioritized list for use in treatment of an individual. When molecular profiling results are obtained, e.g., differential expression or mutation of a gene or gene product, the results can be compared against the database to guide treatment selection. The set of rules in the database can be updated as new treatments and new treatment data become available. In some embodiments, the rules database is updated continuously. In some embodiments, the rules database is updated on a periodic basis. Any relevant correlative or comparative approach can be used to compare the molecular profiling results to the rules database. In one embodiment, a gene or gene product is identified as differentially expressed by molecular profiling. The rules database is queried to select entries for that gene or gene product. Treatment selection information selected from the rules database is extracted and used to select a treatment. The information, e.g., to recommend or not recommend a particular treatment, can be dependent on whether the gene or gene product is over or underexpressed, or has other abnormalities at the genetic or protein levels as compared to a reference. In some cases, multiple rules and treatments may be pulled from a database comprising the comprehensive rules set depending on the results of the molecular profiling. In some embodiments, the treatment options are presented in a prioritized list. In some embodiments, the treatment options are presented without prioritization information. In either case, an individual, e.g., the treating physician or similar caregiver may choose from the available options.

The methods described herein are used to prolong survival of a subject by providing personalized treatment. In some embodiments, the subject has been previously treated with one or more therapeutic agents to treat the disease, e.g., a cancer. The cancer may be refractory to one of these agents, e.g., by acquiring drug resistance mutations. In some embodiments, the cancer is metastatic. In some embodiments, the subject has not previously been treated with one or more therapeutic agents identified by the method. Using molecular profiling, candidate treatments can be selected regardless of the stage, anatomical location, or anatomical origin of the cancer cells.

Progression-free survival (PFS) denotes the chances of staying free of disease progression for an individual or a group of individuals suffering from a disease, e.g., a cancer, after initiating a course of treatment. It can refer to the percentage of individuals in a group whose disease is likely to remain stable (e.g., not show signs of progression) after a specified duration of time. Progression-free survival rates are an indication of the effectiveness of a particular treatment. Similarly, disease-free survival (DFS) denotes the chances of staying free of disease after initiating a particular treatment for an individual or a group of individuals suffering from a cancer. It can refer to the percentage of individuals in a group who are likely to be free of disease after a specified duration of time. Disease-free survival rates are an indication of the effectiveness of a particular treatment. Treatment strategies can be compared on the basis of the PFS or DFS that is achieved in similar groups of patients. Disease-free survival is often used with the term overall survival when cancer survival is described.

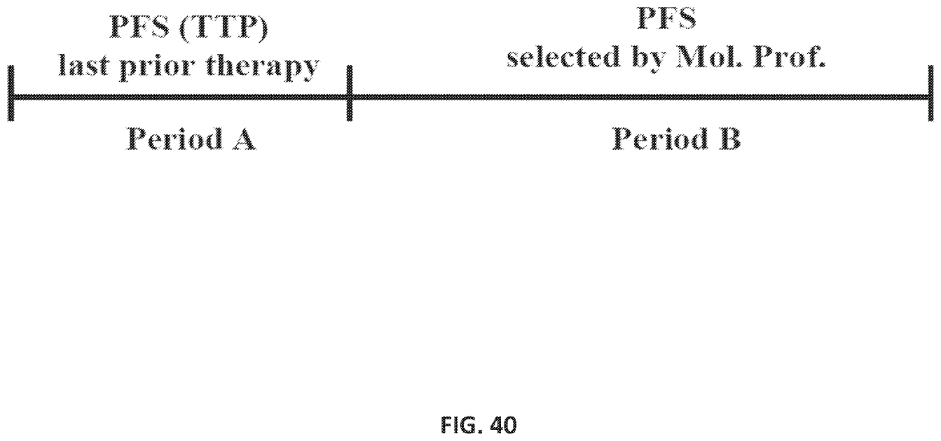

The candidate treatment selected by molecular profiling according to the invention can be compared to a non-molecular profiling selected treatment by comparing the progression free survival (PFS) using therapy selected by molecular profiling (period B) with PFS for the most recent therapy on which the patient has just progressed (period A). See FIG. 40. In one setting, a PFS(B)/PFS(A) ratio ≥1.3 was used to indicate that the molecular profiling selected therapy provides benefit for patient (Robert Temple, Clinical measurement in drug evaluation. Edited by Wu Ningano and G. T. Thicker John Wiley and Sons Ltd. 1995; Von Hoff D. D. Clin Can Res. 4: 1079, 1999: Dhani et al. Clin Cancer Res. 15: 118-123, 2009). Other methods of comparing the treatment selected by molecular profiling to a non-molecular profiling selected treatment include determining response rate (RECIST) and percent of patients without progression or death at 4 months. The term “about” as used in the context of a numerical value for PFS means a variation of +/−ten percent (10%) relative to the numerical value. The PFS from a treatment selected by molecular profiling can be extended by at least 10%, 15%, 20%, 30%, 40%, 50%, 60%, 70%, 80%, or at least 90% as compared to a non-molecular profiling selected treatment. In some embodiments, the PFS from a treatment selected by molecular profiling can be extended by at least 100%, 150%, 200%, 300%, 400%, 500%, 600%, 700%, 800%, 900%, or at least about 1000% as compared to a non-molecular profiling selected treatment. In yet other embodiments, the PFS ratio (PFS on molecular profiling selected therapy or new treatment/PFS on prior therapy or treatment) is at least about 1.3. In yet other embodiments, the PFS ratio is at least about 1.1, 1.2, 1.3, 1.4, 1.5, 1.6, 1.7, 1.8, 1.9, or 2.0. In yet other embodiments, the PFS ratio is at least about 3, 4, 5, 6, 7, 8, 9 or 10.

Similarly, the DFS can be compared in patients whose treatment is selected with or without molecular profiling. In embodiments, DFS from a treatment selected by molecular profiling is extended by at least 10%, 15%, 20%, 30%, 40%, 50%, 60%, 70%, 80%, or at least 90% as compared to a non-molecular profiling selected treatment. In some embodiments, the DFS from a treatment selected by molecular profiling can be extended by at least 100%, 150%, 200%, 300%, 400%, 500%, 600%, 700%, 800%, 900%, or at least about 1000% as compared to a non-molecular profiling selected treatment. In yet other embodiments, the DFS ratio (DFS on molecular profiling selected therapy or new treatment/DFS on prior therapy or treatment) is at least about 1.3. In yet other embodiments, the DFS ratio is at least about 1.1, 1.2, 1.3, 1.4, 1.5, 1.6, 1.7, 1.8, 1.9, or 2.0. In yet other embodiments, the DFS ratio is at least about 3, 4, 5, 6, 7, 8, 9 or 10.

In some embodiments, the candidate treatment of the invention will not increase the PFS ratio or the DFS ratio in the patient, nevertheless molecular profiling provides invaluable patient benefit. For example, in some instances no preferable treatment has been identified for the patient. In such cases, molecular profiling provides a method to identify a candidate treatment where none is currently identified. The molecular profiling may extend PFS, DFS or lifespan by at least 1 week, 2 weeks, 3 weeks, 4 weeks, 1 month, 5 weeks, 6 weeks, 7 weeks, 8 weeks, 2 months, 9 weeks, 10 weeks, 11 weeks, 12 weeks, 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months or 2 years. The molecular profiling may extend PFS, DFS or lifespan by at least 2½ years, 3 years, 4 years, 5 years, or more. In some embodiments, the methods of the invention improve outcome so that patient is in remission.

The effectiveness of a treatment can be monitored by other measures. A complete response (CR) comprises a complete disappearance of the disease: no disease is evident on examination, scans or other tests. A partial response (PR) refers to some disease remaining in the body, but there has been a decrease in size or number of the lesions by 30% or more. Stable disease (SD) refers to a disease that has remained relatively unchanged in size and number of lesions. Generally, less than a 50% decrease or a slight increase in size would be described as stable disease. Progressive disease (PD) means that the disease has increased in size or number on treatment. In some embodiments, molecular profiling according to the invention results in a complete response or partial response. In some embodiments, the methods of the invention result in stable disease. In some embodiments, the invention is able to achieve stable disease where non-molecular profiling results in progressive disease.

### Computer Systems

The practice of the present invention may also employ conventional biology methods, software and systems. Computer software products of the invention typically include computer readable medium having computer-executable instructions for performing the logic steps of the method of the invention. Suitable computer readable medium include floppy disk, CD-ROM/DVD/DVD-ROM, hard-disk drive, flash memory, ROM/RAM, magnetic tapes and etc. The computer executable instructions may be written in a suitable computer language or combination of several languages. Basic computational biology methods are described in, for example Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouelette and Bzevanis Bioinformatics: A Practical Guide for Analysis of Gene and Proteins (Wiley & Sons, Inc., 2.sup.nd ed., 2001). See U.S. Pat. No. 6,420,108.

The present invention may also make use of various computer program products and software for a variety of purposes, such as probe design, management of data, analysis, and instrument operation. See, U.S. Pat. Nos. 5,593,839, 5,795,716, 5,733,729, 5,974,164, 6,066,454, 6,090,555, 6,185,561, 6,188,783, 6,223,127, 6,229,911 and 6,308,170.

Additionally, the present invention relates to embodiments that include methods for providing genetic information over networks such as the Internet as shown in U.S. Ser. Nos. 10/197,621, 10/063,559 (U.S. Publication Number 20020183936), 10/065,856, 10/065,868, 10/328,818, 10/328,872, 10/423,403, and 60/482,389. For example, one or more molecular profiling techniques can be performed in one location, e.g., a city, state, country or continent, and the results can be transmitted to a different city, state, country or continent. Treatment selection can then be made in whole or in part in the second location. The methods of the invention comprise transmittal of information between different locations.

Conventional data networking, application development and other functional aspects of the systems (and components of the individual operating components of the systems) may not be described in detail herein but are part of the invention. Furthermore, the connecting lines shown in the various figures contained herein are intended to represent illustrative functional relationships and/or physical couplings between the various elements. It should be noted that many alternative or additional functional relationships or physical connections may be present in a practical system.

The various system components discussed herein may include one or more of the following: a host server or other computing systems including a processor for processing digital data; a memory coupled to the processor for storing digital data; an input digitizer coupled to the processor for inputting digital data; an application program stored in the memory and accessible by the processor for directing processing of digital data by the processor; a display device coupled to the processor and memory for displaying information derived from digital data processed by the processor; and a plurality of databases. Various databases used herein may include: patient data such as family history, demography and environmental data, biological sample data, prior treatment and protocol data, patient clinical data, molecular profiling data of biological samples, data on therapeutic drug agents and/or investigative drugs, a gene library, a disease library, a drug library, patient tracking data, file management data, financial management data, billing data and/or like data useful in the operation of the system. As those skilled in the art will appreciate, user computer may include an operating system (e.g., Windows NT, 95/98/2000, OS2, UNIX, Linux, Solaris, MacOS, etc.) as well as various conventional support software and drivers typically associated with computers. The computer may include any suitable personal computer, network computer, workstation, minicomputer, mainframe or the like. User computer can be in a home or medical/business environment with access to a network. In an illustrative embodiment, access is through a network or the Internet through a commercially-available web-browser software package.

As used herein, the term “network” shall include any electronic communications means which incorporates both hardware and software components of such. Communication among the parties may be accomplished through any suitable communication channels, such as, for example, a telephone network, an extranet, an intranet, Internet, point of interaction device, personal digital assistant (e.g., Palm Pilot®, Blackberry®), cellular phone, kiosk, etc.), online communications, satellite communications, off-line communications, wireless communications, transponder communications, local area network (LAN), wide area network (WAN), networked or linked devices, keyboard, mouse and/or any suitable communication or data input modality. Moreover, although the system is frequently described herein as being implemented with TCP/IP communications protocols, the system may also be implemented using IPX, Appletalk, IP-6, NetBIOS, OSI or any number of existing or future protocols. If the network is in the nature of a public network, such as the Internet, it may be advantageous to presume the network to be insecure and open to eavesdroppers. Specific information related to the protocols, standards, and application software used in connection with the Internet is generally known to those skilled in the art and, as such, need not be detailed herein. See, for example, DILIP NAIK, INTERNET STANDARDS AND PROTOCOLS (1998); JAVA 2 COMPLETE, various authors, (Sybex 1999); DEBORAH RAY AND ERIC RAY, MASTERING HTML 4.0 (1997); and LOSHIN, TCP/IP CLEARLY EXPLAINED (1997) and DAVID GOURLEY AND BRIAN TOTTY, HTTP, THE DEFINITIVE GUIDE (2002), the contents of which are hereby incorporated by reference.

The various system components may be independently, separately or collectively suitably coupled to the network via data links which includes, for example, a connection to an Internet Service Provider (ISP) over the local loop as is typically used in connection with standard modem communication, cable modem, Dish networks, ISDN, Digital Subscriber Line (DSL), or various wireless communication methods, see, e.g., GILBERT HELD, UNDERSTANDING DATA COMMUNICATIONS (1996), which is hereby incorporated by reference. It is noted that the network may be implemented as other types of networks, such as an interactive television (ITV) network. Moreover, the system contemplates the use, sale or distribution of any goods, services or information over any network having similar functionality described herein.

As used herein, “transmit” may include sending electronic data from one system component to another over a network connection. Additionally, as used herein, “data” may include encompassing information such as commands, queries, files, data for storage, and the like in digital or any other form.

The system contemplates uses in association with web services, utility computing, pervasive and individualized computing, security and identity solutions, autonomic computing, commodity computing, mobility and wireless solutions, open source, biometrics, grid computing and/or mesh computing.

Any databases discussed herein may include relational, hierarchical, graphical, or object-oriented structure and/or any other database configurations. Common database products that may be used to implement the databases include DB2 by IBM (White Plains, N.Y.), various database products available from Oracle Corporation (Redwood Shores, Calif.), Microsoft Access or Microsoft SQL Server by Microsoft Corporation (Redmond, Wash.), or any other suitable database product. Moreover, the databases may be organized in any suitable manner, for example, as data tables or lookup tables. Each record may be a single file, a series of files, a linked series of data fields or any other data structure. Association of certain data may be accomplished through any desired data association technique such as those known or practiced in the art. For example, the association may be accomplished either manually or automatically. Automatic association techniques may include, for example, a database search, a database merge, GREP, AGREP, SQL, using a key field in the tables to speed searches, sequential searches through all the tables and files, sorting records in the file according to a known order to simplify lookup, and/or the like. The association step may be accomplished by a database merge function, for example, using a “key field” in pre-selected databases or data sectors.

More particularly, a “key field” partitions the database according to the high-level class of objects defined by the key field. For example, certain types of data may be designated as a key field in a plurality of related data tables and the data tables may then be linked on the basis of the type of data in the key field. The data corresponding to the key field in each of the linked data tables is preferably the same or of the same type. However, data tables having similar, though not identical, data in the key fields may also be linked by using AGREP, for example. In accordance with one embodiment, any suitable data storage technique may be used to store data without a standard format. Data sets may be stored using any suitable technique, including, for example, storing individual files using an ISO/IEC 7816-4 file structure; implementing a domain whereby a dedicated file is selected that exposes one or more elementary files containing one or more data sets; using data sets stored in individual files using a hierarchical filing system; data sets stored as records in a single file (including compression, SQL accessible, hashed vione or more keys, numeric, alphabetical by first tuple, etc.); Binary Large Object (BLOB); stored as ungrouped data elements encoded using ISO/IEC 7816-6 data elements; stored as ungrouped data elements encoded using ISO/IEC Abstract Syntax Notation (ASN.1) as in ISO/IEC 8824 and 8825; and/or other proprietary techniques that may include fractal compression methods, image compression methods, etc.

In one illustrative embodiment, the ability to store a wide variety of information in different formats is facilitated by storing the information as a BLOB. Thus, any binary information can be stored in a storage space associated with a data set. The BLOB method may store data sets as ungrouped data elements formatted as a block of binary via a fixed memory offset using either fixed storage allocation, circular queue techniques, or best practices with respect to memory management (e.g., paged memory, least recently used, etc.). By using BLOB methods, the ability to store various data sets that have different formats facilitates the storage of data by multiple and unrelated owners of the data sets. For example, a first data set which may be stored may be provided by a first party, a second data set which may be stored may be provided by an unrelated second party, and yet a third data set which may be stored, may be provided by a third party unrelated to the first and second party. Each of these three illustrative data sets may contain different information that is stored using different data storage formats and/or techniques. Further, each data set may contain subsets of data that also may be distinct from other subsets.

As stated above, in various embodiments, the data can be stored without regard to a common format. However, in one illustrative embodiment, the data set (e.g., BLOB) may be annotated in a standard manner when provided for manipulating the data. The annotation may comprise a short header, trailer, or other appropriate indicator related to each data set that is configured to convey information useful in managing the various data sets. For example, the annotation may be called a “condition header”, “header”, “trailer”, or “status”, herein, and may comprise an indication of the status of the data set or may include an identifier correlated to a specific issuer or owner of the data. Subsequent bytes of data may be used to indicate for example, the identity of the issuer or owner of the data, user, transaction/membership account identifier or the like. Each of these condition annotations are further discussed herein.

The data set annotation may also be used for other types of status information as well as various other purposes. For example, the data set annotation may include security information establishing access levels. The access levels may, for example, be configured to permit only certain individuals, levels of employees, companies, or other entities to access data sets, or to permit access to specific data sets based on the transaction, issuer or owner of data, user or the like. Furthermore, the security information may restrict/permit only certain actions such as accessing, modifying, and/or deleting data sets. In one example, the data set annotation indicates that only the data set owner or the user are permitted to delete a data set, various identified users may be permitted to access the data set for reading, and others are altogether excluded from accessing the data set. However, other access restriction parameters may also be used allowing various entities to access a data set with various permission levels as appropriate. The data, including the header or trailer may be received by a standalone interaction device configured to add, delete, modify, or augment the data in accordance with the header or trailer.

One skilled in the art will also appreciate that, for security reasons, any databases, systems, devices, servers or other components of the system may consist of any combination thereof at a single location or at multiple locations, wherein each database or system includes any of various suitable security features, such as firewalls, access codes, encryption, decryption, compression, decompression, and/or the like.

The computing unit of the web client may be further equipped with an Internet browser connected to the Internet or an intranet using standard dial-up, cable, DSL or any other Internet protocol known in the art. Transactions originating at a web client may pass through a firewall in order to prevent unauthorized access from users of other networks. Further, additional firewalls may be deployed between the varying components of CMS to further enhance security.

Firewall may include any hardware and/or software suitably configured to protect CMS components and/or enterprise computing resources from users of other networks. Further, a firewall may be configured to limit or restrict access to various systems and components behind the firewall for web clients connecting through a web server. Firewall may reside in varying configurations including Stateful Inspection, Proxy based and Packet Filtering among others. Firewall may be integrated within an web server or any other CMS components or may further reside as a separate entity.

The computers discussed herein may provide a suitable website or other Internet-based graphical user interface which is accessible by users. In one embodiment, the Microsoft Internet Information Server (IIS), Microsoft Transaction Server (MTS), and Microsoft SQL Server, are used in conjunction with the Microsoft operating system, Microsoft NT web server software, a Microsoft SQL Server database system, and a Microsoft Commerce Server. Additionally, components such as Access or Microsoft SQL Server, Oracle, Sybase, Informix MySQL, Interbase, etc., may be used to provide an Active Data Object (ADO) compliant database management system.

Any of the communications, inputs, storage, databases or displays discussed herein may be facilitated through a website having web pages. The term “web page” as it is used herein is not meant to limit the type of documents and applications that might be used to interact with the user. For example, a typical website might include, in addition to standard HTML documents, various forms, Java applets, JavaScript, active server pages (ASP), common gateway interface scripts (CGI), extensible markup language (XML), dynamic HTML, cascading style sheets (CSS), helper applications, plug-ins, and the like. A server may include a web service that receives a request from a web server, the request including a URL (http://yahoo.com/stockquotes/ge) and an IP address (123.56.789.234). The web server retrieves the appropriate web pages and sends the data or applications for the web pages to the IP address. Web services are applications that are capable of interacting with other applications over a communications means, such as the internet. Web services are typically based on standards or protocols such as XML, XSLT, SOAP, WSDL and UDDI. Web services methods are well known in the art, and are covered in many standard texts. See, e.g., ALEX NGHIEM, IT WEB SERVICES: A ROADMAP FOR THE ENTERPRISE (2003), hereby incorporated by reference.

The web-based clinical database for the system and method of the present invention preferably has the ability to upload and store clinical data files in native formats and is searchable on any clinical parameter. The database is also scalable and may use an EAV data model (metadata) to enter clinical annotations from any study for easy integration with other studies. In addition, the web-based clinical database is flexible and may be XML and XSLT enabled to be able to add user customized questions dynamically. Further, the database includes exportability to CDISC ODM.

Practitioners will also appreciate that there are a number of methods for displaying data within a browser-based document. Data may be represented as standard text or within a fixed list, scrollable list, drop-down list, editable text field, fixed text field, pop-up window, and the like. Likewise, there are a number of methods available for modifying data in a web page such as, for example, free text entry using a keyboard, selection of menu items, check boxes, option boxes, and the like.

The system and method may be described herein in terms of functional block components, screen shots, optional selections and various processing steps. It should be appreciated that such functional blocks may be realized by any number of hardware and/or software components configured to perform the specified functions. For example, the system may employ various integrated circuit components, e.g., memory elements, processing elements, logic elements, look-up tables, and the like, which may carry out a variety of functions under the control of one or more microprocessors or other control devices. Similarly, the software elements of the system may be implemented with any programming or scripting language such as C, C++, Macromedia Cold Fusion, Microsoft Active Server Pages, Java, COBOL, assembler, PERL, Visual Basic, SQL Stored Procedures, extensible markup language (XML), with the various algorithms being implemented with any combination of data structures, objects, processes, routines or other programming elements. Further, it should be noted that the system may employ any number of conventional techniques for data transmission, signaling, data processing, network control, and the like. Still further, the system could be used to detect or prevent security issues with a client-side scripting language, such as JavaScript, VBScript or the like. For a basic introduction of cryptography and network security, see any of the following references: (1) “Applied Cryptography: Protocols, Algorithms, And Source Code In C,” by Bruce Schneier, published by John Wiley & Sons (second edition, 1995); (2) “Java Cryptography” by Jonathan Knudson, published by O'Reilly & Associates (1998); (3) “Cryptography & Network Security: Principles & Practice” by William Stallings, published by Prentice Hall; all of which are hereby incorporated by reference.

As used herein, the term “end user”, “consumer”, “customer”, “client”, “treating physician”, “hospital”, or “business” may be used interchangeably with each other, and each shall mean any person, entity, machine, hardware, software or business. Each participant is equipped with a computing device in order to interact with the system and facilitate online data access and data input. The customer has a computing unit in the form of a personal computer, although other types of computing units may be used including laptops, notebooks, hand held computers, set-top boxes, cellular telephones, touch-tone telephones and the like. The owner/operator of the system and method of the present invention has a computing unit implemented in the form of a computer-server, although other implementations are contemplated by the system including a computing center shown as a main frame computer, a mini-computer, a PC server, a network of computers located in the same of different geographic locations, or the like. Moreover, the system contemplates the use, sale or distribution of any goods, services or information over any network having similar functionality described herein.

In one illustrative embodiment, each client customer may be issued an “account” or “account number”. As used herein, the account or account number may include any device, code, number, letter, symbol, digital certificate, smart chip, digital signal, analog signal, biometric or other identifier/indicia suitably configured to allow the consumer to access, interact with or communicate with the system (e.g., one or more of an authorization/access code, personal identification number (PIN), Internet code, other identification code, and/or the like). The account number may optionally be located on or associated with a charge card, credit card, debit card, prepaid card, embossed card, smart card, magnetic stripe card, bar code card, transponder, radio frequency card or an associated account. The system may include or interface with any of the foregoing cards or devices, or a fob having a transponder and RFID reader in RF communication with the fob. Although the system may include a fob embodiment, the invention is not to be so limited. Indeed, system may include any device having a transponder which is configured to communicate with RFID reader via RF communication. Typical devices may include, for example, a key ring, tag, card, cell phone, wristwatch or any such form capable of being presented for interrogation. Moreover, the system, computing unit or device discussed herein may include a “pervasive computing device,” which may include a traditionally non-computerized device that is embedded with a computing unit. The account number may be distributed and stored in any form of plastic, electronic, magnetic, radio frequency, wireless, audio and/or optical device capable of transmitting or downloading data from itself to a second device.

As will be appreciated by one of ordinary skill in the art, the system may be embodied as a customization of an existing system, an add-on product, upgraded software, a standalone system, a distributed system, a method, a data processing system, a device for data processing, and/or a computer program product. Accordingly, the system may take the form of an entirely software embodiment, an entirely hardware embodiment, or an embodiment combining aspects of both software and hardware. Furthermore, the system may take the form of a computer program product on a computer-readable storage medium having computer-readable program code means embodied in the storage medium. Any suitable computer-readable storage medium may be used, including hard disks, CD-ROM, optical storage devices, magnetic storage devices, and/or the like.

The system and method is described herein with reference to screen shots, block diagrams and flowchart illustrations of methods, apparatus (e.g., systems), and computer program products according to various embodiments. It will be understood that each functional block of the block diagrams and the flowchart illustrations, and combinations of functional blocks in the block diagrams and flowchart illustrations, respectively, can be implemented by computer program instructions.

These computer program instructions may be loaded onto a general purpose computer, special purpose computer, or other programmable data processing apparatus to produce a machine, such that the instructions that execute on the computer or other programmable data processing apparatus create means for implementing the functions specified in the flowchart block or blocks. These computer program instructions may also be stored in a computer-readable memory that can direct a computer or other programmable data processing apparatus to function in a particular manner, such that the instructions stored in the computer-readable memory produce an article of manufacture including instruction means which implement the function specified in the flowchart block or blocks. The computer program instructions may also be loaded onto a computer or other programmable data processing apparatus to cause a series of operational steps to be performed on the computer or other programmable apparatus to produce a computer-implemented process such that the instructions which execute on the computer or other programmable apparatus provide steps for implementing the functions specified in the flowchart block or blocks.

Accordingly, functional blocks of the block diagrams and flowchart illustrations support combinations of means for performing the specified functions, combinations of steps for performing the specified functions, and program instruction means for performing the specified functions. It will also be understood that each functional block of the block diagrams and flowchart illustrations, and combinations of functional blocks in the block diagrams and flowchart illustrations, can be implemented by either special purpose hardware-based computer systems which perform the specified functions or steps, or suitable combinations of special purpose hardware and computer instructions. Further, illustrations of the process flows and the descriptions thereof may make reference to user windows, web pages, websites, web forms, prompts, etc. Practitioners will appreciate that the illustrated steps described herein may comprise in any number of configurations including the use of windows, web pages, web forms, popup windows, prompts and the like. It should be further appreciated that the multiple steps as illustrated and described may be combined into single web pages and/or windows but have been expanded for the sake of simplicity. In other cases, steps illustrated and described as single process steps may be separated into multiple web pages and/or windows but have been combined for simplicity.

### Molecular Profiling Methods

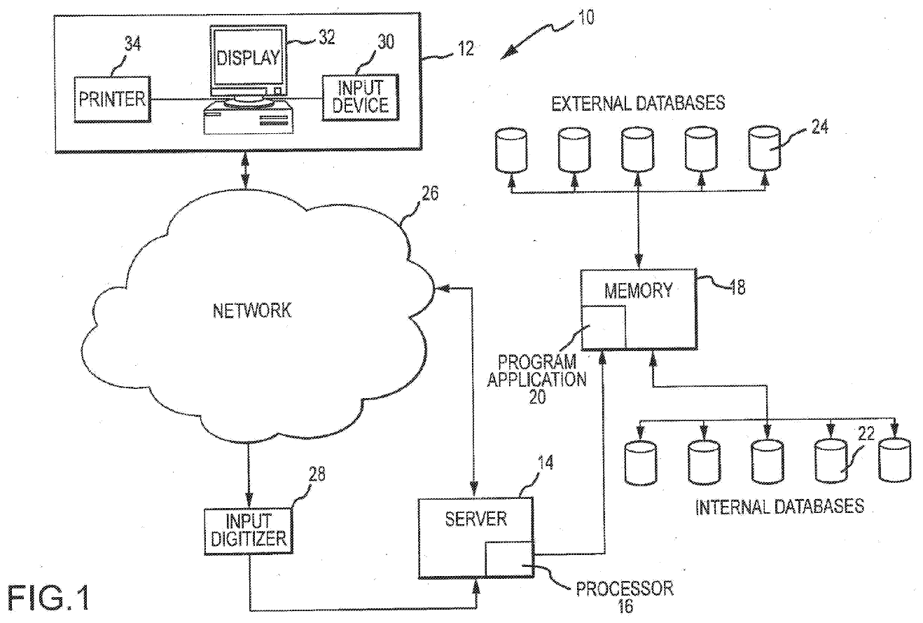

FIG. 1 illustrates a block diagram of an illustrative embodiment of a system 10 for determining individualized medical intervention for a particular disease state that uses molecular profiling of a patient's biological specimen. System 10 includes a user interface 12, a host server 14 including a processor 16 for processing data, a memory 18 coupled to the processor, an application program 20 stored in the memory 18 and accessible by the processor 16 for directing processing of the data by the processor 16, a plurality of internal databases 22 and external databases 24, and an interface with a wired or wireless communications network 26 (such as the Internet, for example). System 10 may also include an input digitizer 28 coupled to the processor 16 for inputting digital data from data that is received from user interface 12.

User interface 12 includes an input device 30 and a display 32 for inputting data into system 10 and for displaying information derived from the data processed by processor 16. User interface 12 may also include a printer 34 for printing the information derived from the data processed by the processor 16 such as patient reports that may include test results for targets and proposed drug therapies based on the test results.

Internal databases 22 may include, but are not limited to, patient biological sample/specimen information and tracking, clinical data, patient data, patient tracking, file management, study protocols, patient test results from molecular profiling, and billing information and tracking. External databases 24 may include, but are not limited to, drug libraries, gene libraries, disease libraries, and public and private databases such as UniGene, OMIM, GO, TIGR, GenBank, KEGG and Biocarta.

Various methods may be used in accordance with system 10. FIG. 2 shows a flowchart of an illustrative embodiment of a method 50 for determining individualized medical intervention for a particular disease state that uses molecular profiling of a patient's biological specimen that is non disease specific. In order to determine a medical intervention for a particular disease state using molecular profiling that is independent of disease lineage diagnosis (i.e. not single disease restricted), at least one test is performed for at least one target from a biological sample of a diseased patient in step 52. A target is defined as any molecular finding that may be obtained from molecular testing. For example, a target may include one or more genes, one or more gene expressed proteins, one or more molecular mechanisms, and/or combinations of such. For example, the expression level of a target can be determined by the analysis of mRNA levels or the target or gene, or protein levels of the gene. Tests for finding such targets may include, but are not limited, fluorescent in-situ hybridization (FISH), in-situ hybridization (ISH), and other molecular tests known to those skilled in the art. PCR-based methods, such as real-time PCR or quantitative PCR can be used. Furthermore, microarray analysis, such as a comparative genomic hybridization (CGH) micro array, a single nucleotide polymorphism (SNP) microarray, a proteomic array, or antibody array analysis can also be used in the methods disclosed herein. In some embodiments, microarray analysis comprises identifying whether a gene is up-regulated or down-regulated relative to a reference with a significance of p<0.001. Tests or analyses of targets can also comprise immunohistochemical (IHC) analysis. In some embodiments, IHC analysis comprises determining whether 30% or more of a sample is stained, if the staining intensity is +2 or greater, or both.

Furthermore, the methods disclosed herein also including profiling more than one target. For example, the expression of a plurality of genes can be identified. Furthermore, identification of a plurality of targets in a sample can be by one method or by various means. For example, the expression of a first gene can be determined by one method and the expression level of a second gene determined by a different method. Alternatively, the same method can be used to detect the expression level of the first and second gene. For example, the first method can be IHC and the second by microarray analysis, such as detecting the gene expression of a gene.

In some embodiments, molecular profiling can also including identifying a genetic variant, such as a mutation, polymorphism (such as a SNP), deletion, or insertion of a target. For example, identifying a SNP in a gene can be determined by microarray analysis, real-time PCR, or sequencing. Other methods disclosed herein can also be used to identify variants of one or more targets.

Accordingly, one or more of the following may be performed: an IHC analysis in step 54, a microanalysis in step 56, and other molecular tests know to those skilled in the art in step 58.

Biological samples are obtained from diseased patients by taking a biopsy of a tumor, conducting minimally invasive surgery if no recent tumor is available, obtaining a sample of the patient's blood, or a sample of any other biological fluid including, but not limited to, cell extracts, nuclear extracts, cell lysates or biological products or substances of biological origin such as excretions, blood, sera, plasma, urine, sputum, tears, feces, saliva, membrane extracts, and the like.

In step 60, a determination is made as to whether one or more of the targets that were tested for in step 52 exhibit a change in expression compared to a normal reference for that particular target. In one illustrative method of the invention, an IHC analysis may be performed in step 54 and a determination as to whether any targets from the IHC analysis exhibit a change in expression is made in step 64 by determining whether 30% or more of the biological sample cells were +2 or greater staining for the particular target. It will be understood by those skilled in the art that there will be instances where +1 or greater staining will indicate a change in expression in that staining results may vary depending on the technician performing the test and type of target being tested. In another illustrative embodiment of the invention, a micro array analysis may be performed in step 56 and a determination as to whether any targets from the micro array analysis exhibit a change in expression is made in step 66 by identifying which targets are up-regulated or down-regulated by determining whether the fold change in expression for a particular target relative to a normal tissue of origin reference is significant at p<0.001. A change in expression may also be evidenced by an absence of one or more genes, gene expressed proteins, molecular mechanisms, or other molecular findings.

After determining which targets exhibit a change in expression in step 60, at least one non-disease specific agent is identified that interacts with each target having a changed expression in step 70. An agent may be any drug or compound having a therapeutic effect. A non-disease specific agent is a therapeutic drug or compound not previously associated with treating the patient's diagnosed disease that is capable of interacting with the target from the patient's biological sample that has exhibited a change in expression. Some of the non-disease specific agents that have been found to interact with specific targets found in different cancer patients are shown in Table 5 below.

TABLE 5
Illustrative target-drug associations

| Patients | Target(s) Found | Treatment(s) |
|---|---|---|
| Advanced Pancreatic Cancer | HER 2/neu | Trastuzumab |
| Advanced Pancreatic Cancer | EGFR, HIF 1α | Cetuximab, |
| | | Sirolimus |
| Advanced Ovarian Cancer | ERCC3 | Irofulven |
| Advanced Adenoid Cystic | Vitamin D receptors, | Calcitriol, |
| Carcinoma | Androgen receptors | Flutamide |

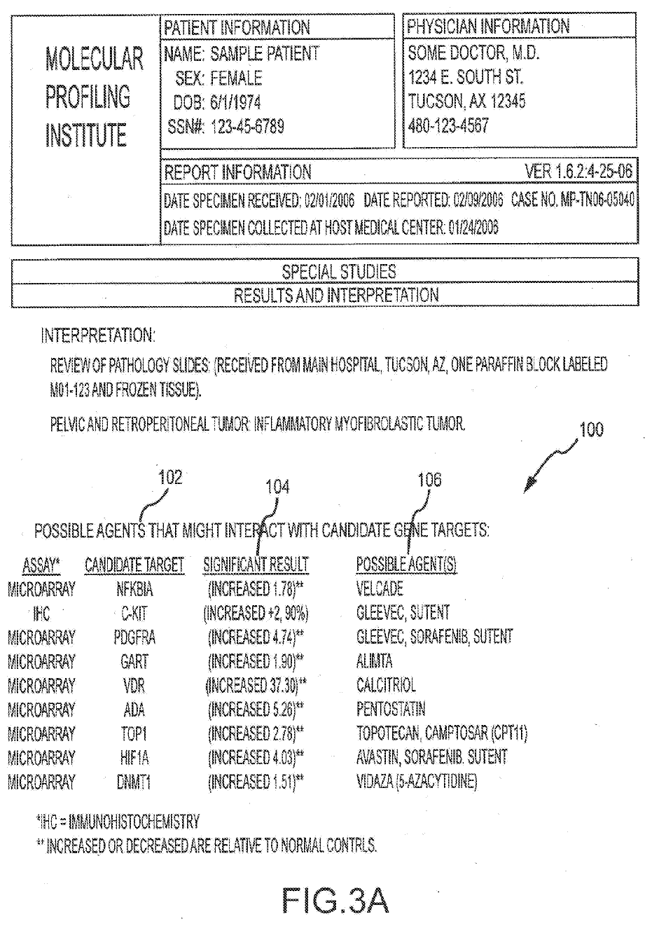

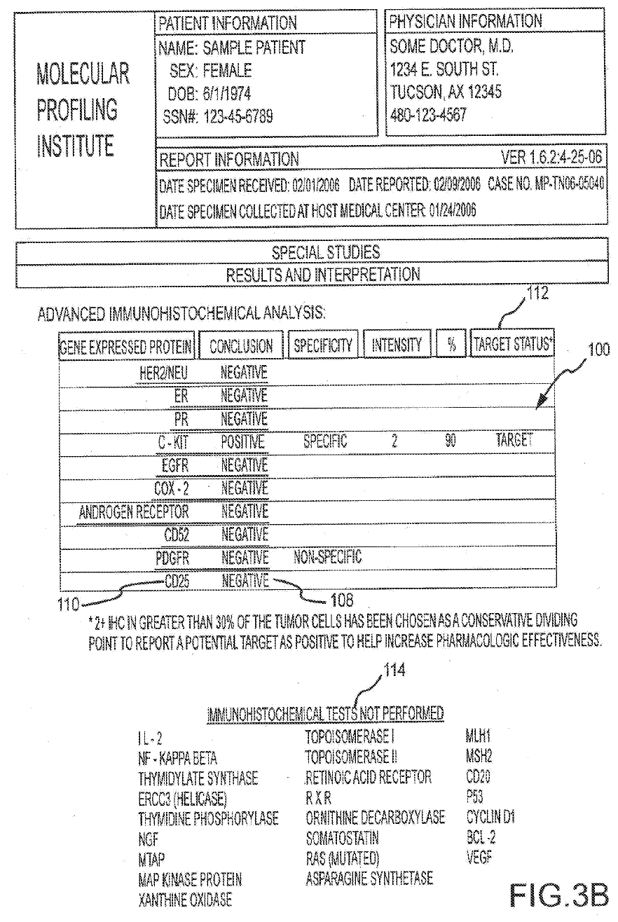

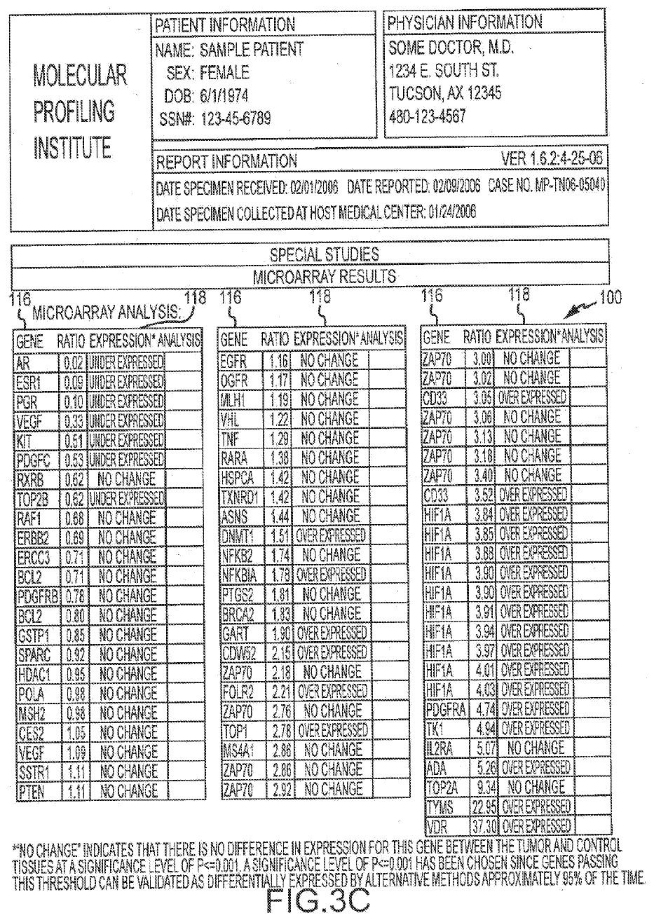

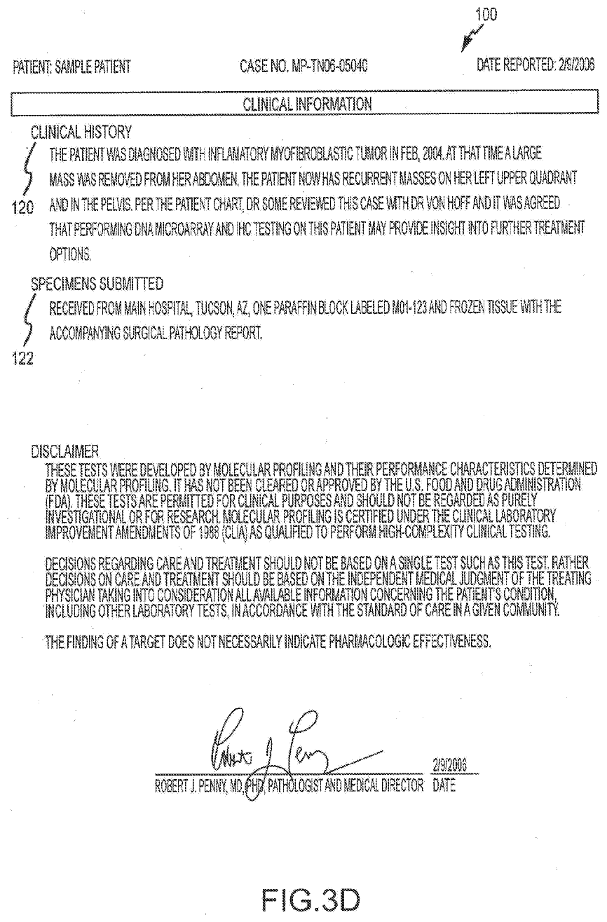

Finally, in step 80, a patient profile report may be provided which includes the patient's test results for various targets and any proposed therapies based on those results. An illustrative patient profile report 100 is shown in FIGS. 3A-3D. Patient profile report 100 shown in FIG. 3A identifies the targets tested 102, those targets tested that exhibited significant changes in expression 104, and proposed non-disease specific agents for interacting with the targets 106. Patient profile report 100 shown in FIG. 3B identifies the results 108 of immunohistochemical analysis for certain gene expressed proteins 110 and whether a gene expressed protein is a molecular target 112 by determining whether 30% or more of the tumor cells were +2 or greater staining. Report 100 also identifies immunohistochemical tests that were not performed 114. Patient profile report 100 shown in FIG. 3C identifies the genes analyzed 116 with a micro array analysis and whether the genes were under expressed or over expressed 118 compared to a reference. Finally, patient profile report 100 shown in FIG. 3D identifies the clinical history 120 of the patient and the specimens that were submitted 122 from the patient. Molecular profiling techniques can be performed anywhere, e.g., a foreign country, and the results sent by network to an appropriate party, e.g., the patient, a physician, lab or other party located remotely.

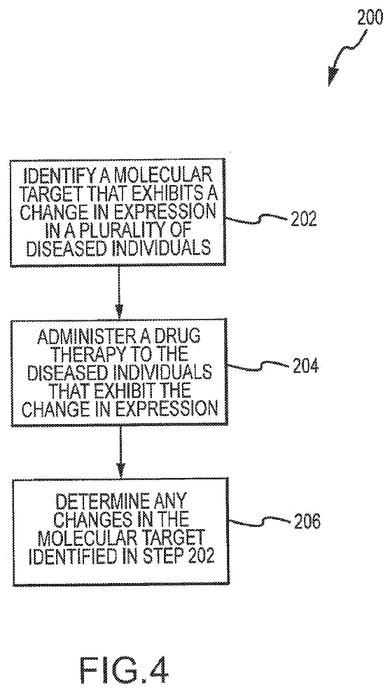

FIG. 4 shows a flowchart of an illustrative embodiment of a method 200 for identifying a drug therapy/agent capable of interacting with a target. In step 202, a molecular target is identified which exhibits a change in expression in a number of diseased individuals. Next, in step 204, a drug therapy/agent is administered to the diseased individuals. After drug therapy/agent administration, any changes in the molecular target identified in step 202 are identified in step 206 in order to determine if the drug therapy/agent administered in step 204 interacts with the molecular targets identified in step 202. If it is determined that the drug therapy/agent administered in step 204 interacts with a molecular target identified in step 202, the drug therapy/agent may be approved for treating patients exhibiting a change in expression of the identified molecular target instead of approving the drug therapy/agent for a particular disease.

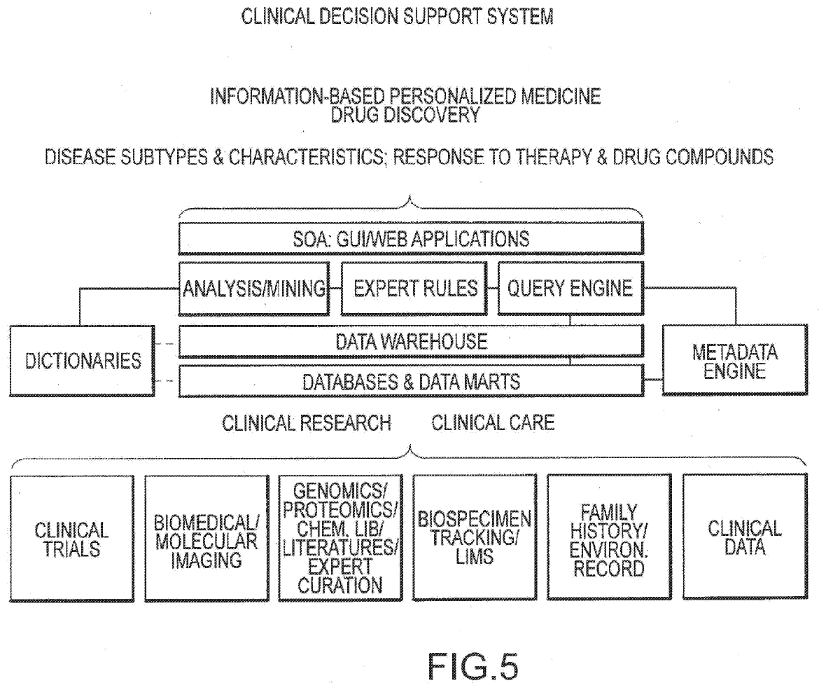

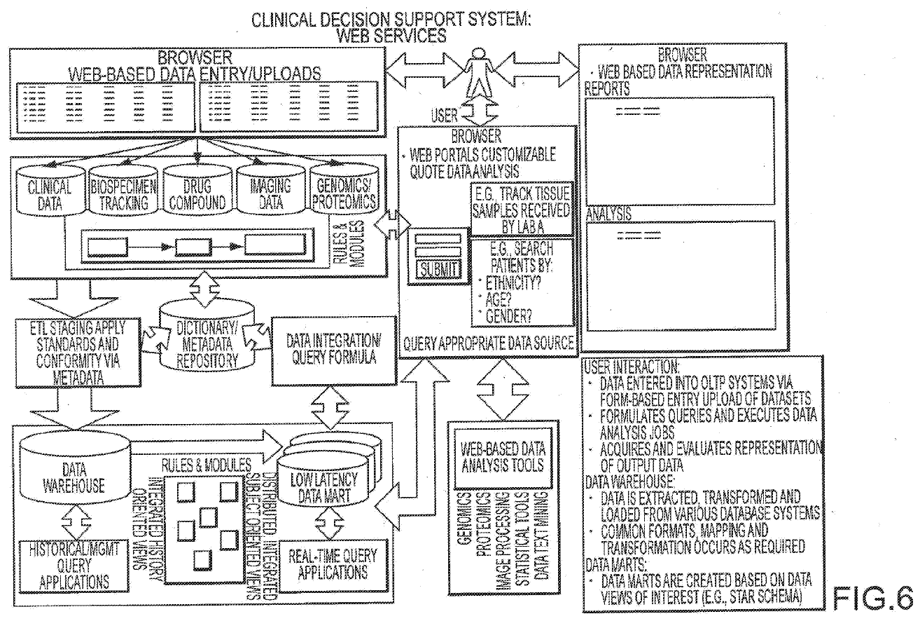

FIGS. 5-14 are flowcharts and diagrams illustrating various parts of an information-based personalized medicine drug discovery system and method in accordance with the present invention. FIG. 5 is a diagram showing an illustrative clinical decision support system of the information-based personalized medicine drug discovery system and method of the present invention. Data obtained through clinical research and clinical care such as clinical trial data, biomedical/molecular imaging data, genomics/proteomics/chemical library/literature/expert curation, biospecimen tracking/LIMS, family history/environmental records, and clinical data are collected and stored as databases and datamarts within a data warehouse. FIG. 6 is a diagram showing the flow of information through the clinical decision support system of the information-based personalized medicine drug discovery system and method of the present invention using web services. A user interacts with the system by entering data into the system via form-based entry/upload of data sets, formulating queries and executing data analysis jobs, and acquiring and evaluating representations of output data. The data warehouse in the web based system is where data is extracted, transformed, and loaded from various database systems. The data warehouse is also where common formats, mapping and transformation occurs. The web based system also includes datamarts which are created based on data views of interest.

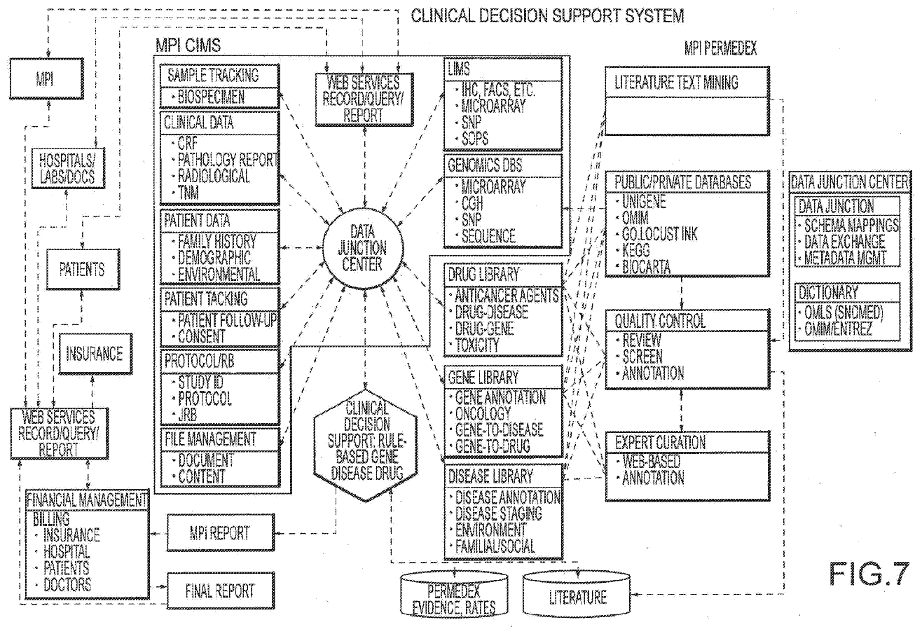

A flow chart of an illustrative clinical decision support system of the information-based personalized medicine drug discovery system and method of the present invention is shown in FIG. 7. The clinical information management system includes the laboratory information management system and the medical information contained in the data warehouses and databases includes medical information libraries, such as drug libraries, gene libraries, and disease libraries, in addition to literature text mining Both the information management systems relating to particular patients and the medical information databases and data warehouses come together at a data junction center where diagnostic information and therapeutic options can be obtained. A financial management system may also be incorporated in the clinical decision support system of the information-based personalized medicine drug discovery system and method of the present invention.

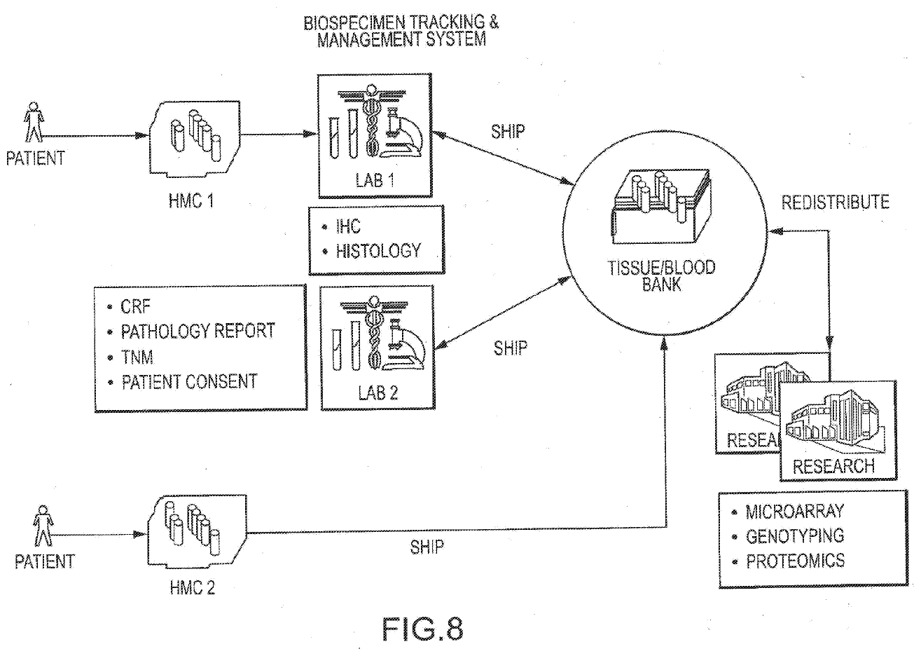

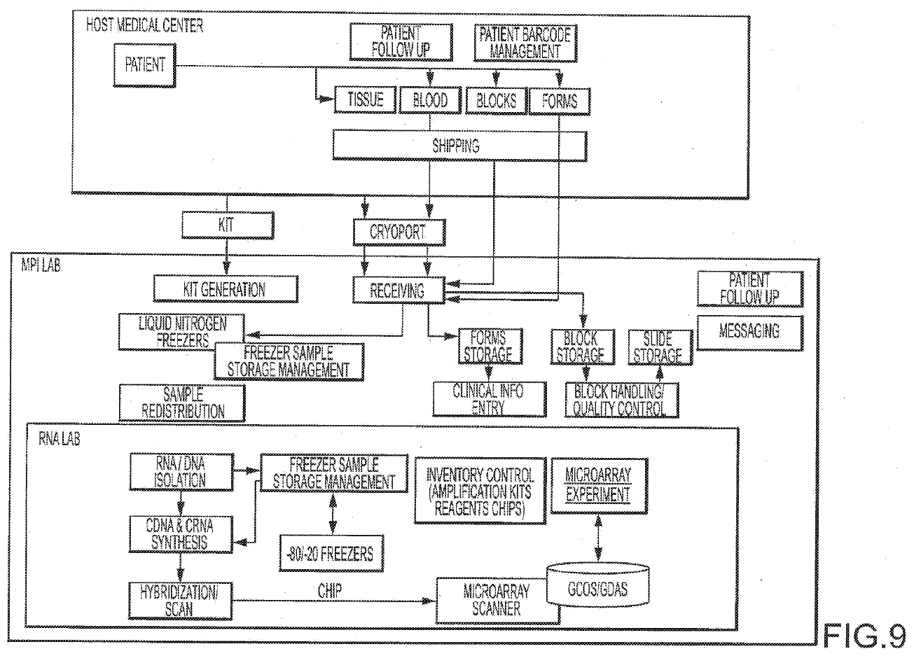

FIG. 8 is a diagram showing an illustrative biospecimen tracking and management system which may be used as part of the information-based personalized medicine drug discovery system and method of the present invention. FIG. 8 shows two host medical centers which forward specimens to a tissue/blood bank. The specimens may go through laboratory analysis prior to shipment. Research may also be conducted on the samples via micro array, genotyping, and proteomic analysis. This information can be redistributed to the tissue/blood bank. FIG. 9 depicts a flow chart of an illustrative biospecimen tracking and management system which may be used with the information-based personalized medicine drug discovery system and method of the present invention. The host medical center obtains samples from patients and then ships the patient samples to a molecular profiling laboratory which may also perform RNA and DNA isolation and analysis.

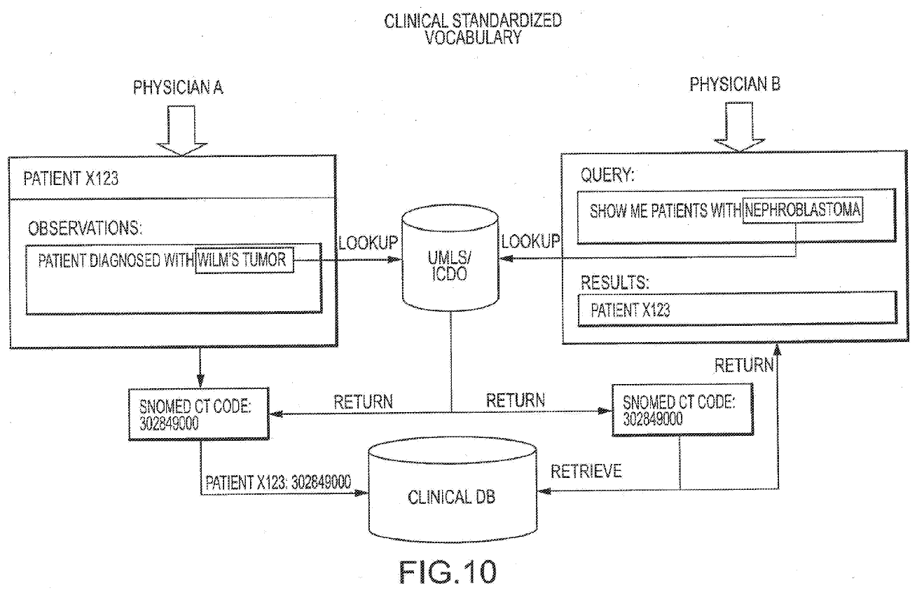

A diagram showing a method for maintaining a clinical standardized vocabulary for use with the information-based personalized medicine drug discovery system and method of the present invention is shown in FIG. 10. FIG. 10 illustrates how physician observations and patient information associated with one physician's patient may be made accessible to another physician to enable the other physician to use the data in making diagnostic and therapeutic decisions for their patients.

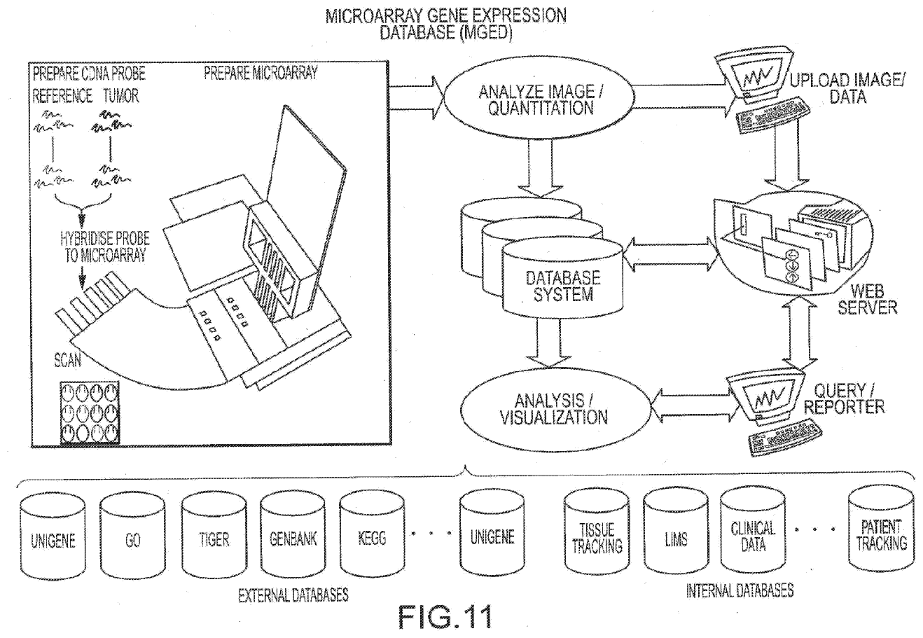

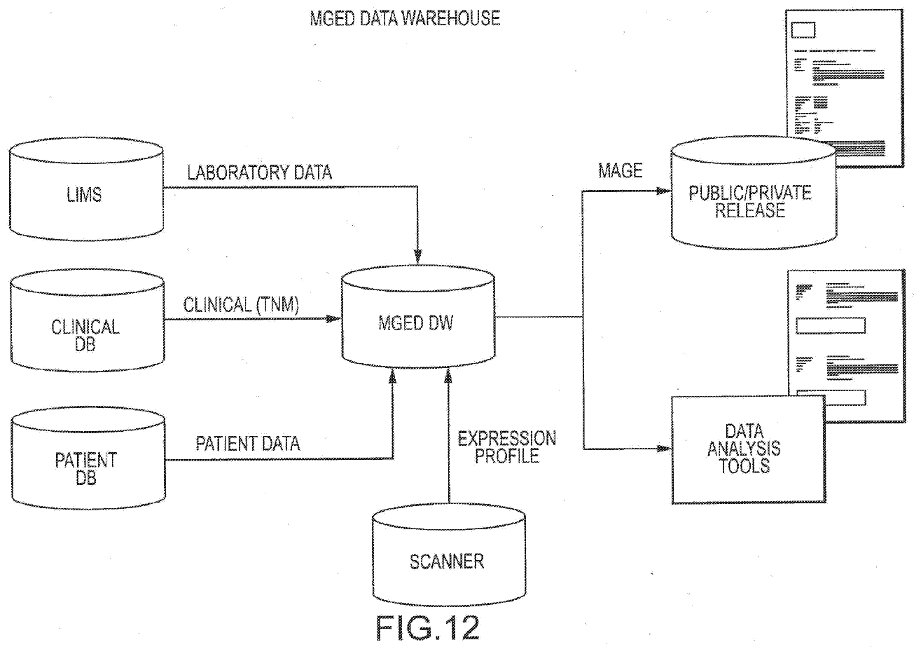

FIG. 11 shows a schematic of an illustrative microarray gene expression database which may be used as part of the information-based personalized medicine drug discovery system and method of the present invention. The micro array gene expression database includes both external databases and internal databases which can be accessed via the web based system. External databases may include, but are not limited to, UniGene, GO, TIGR, GenBank, KEGG. The internal databases may include, but are not limited to, tissue tracking, LIMS, clinical data, and patient tracking. FIG. 12 shows a diagram of an illustrative micro array gene expression database data warehouse which may be used as part of the information-based personalized medicine drug discovery system and method of the present invention. Laboratory data, clinical data, and patient data may all be housed in the micro array gene expression database data warehouse and the data may in turn be accessed by public/private release and used by data analysis tools.

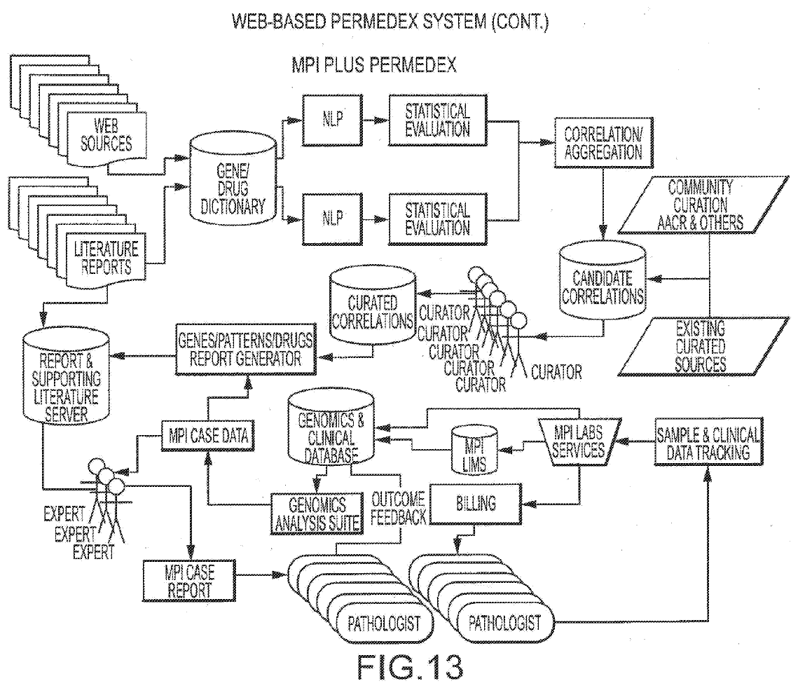

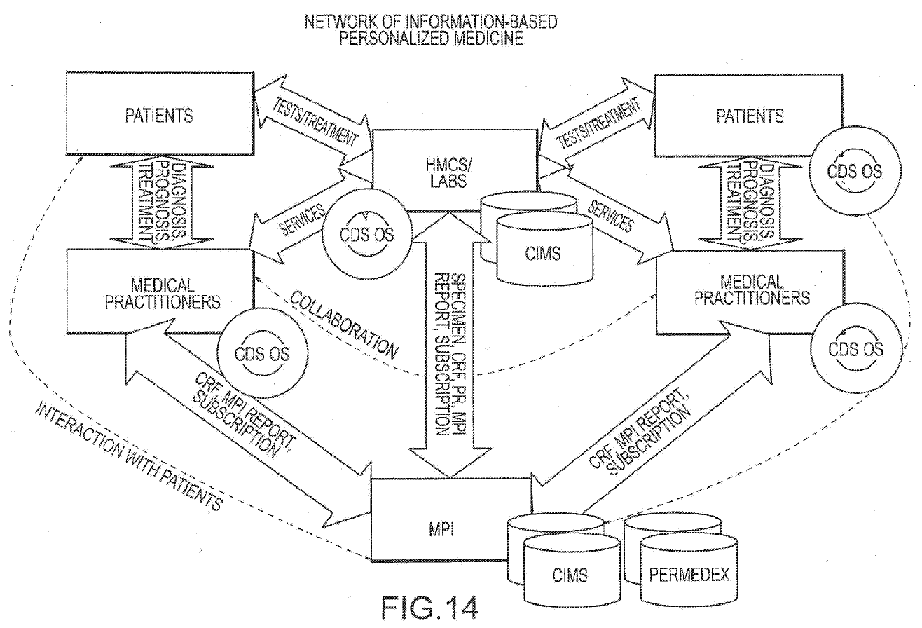

Another schematic showing the flow of information through an information-based personalized medicine drug discovery system and method of the present invention is shown in FIG. 13. Like FIG. 7, the schematic includes clinical information management, medical and literature information management, and financial management of the information-based personalized medicine drug discovery system and method of the present invention. FIG. 14 is a schematic showing an illustrative network of the information-based personalized medicine drug discovery system and method of the present invention. Patients, medical practitioners, host medical centers, and labs all share and exchange a variety of information in order to provide a patient with a proposed therapy or agent based on various identified targets.

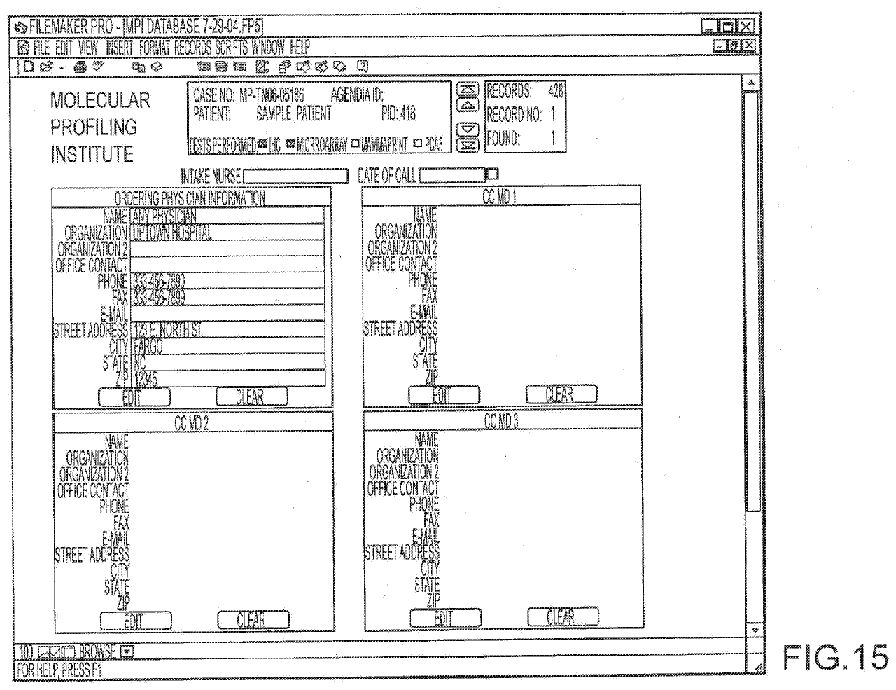

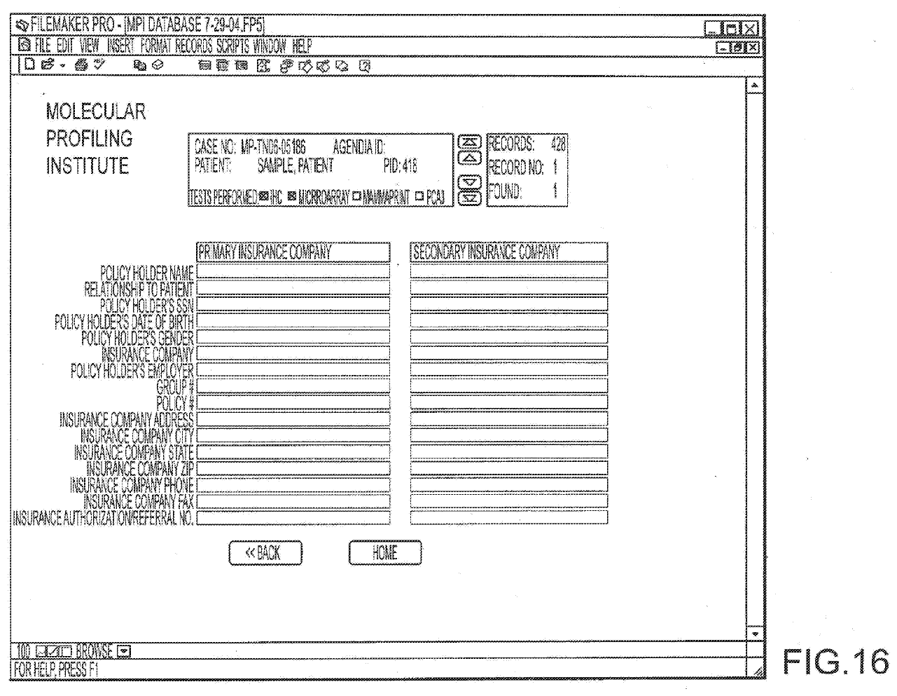

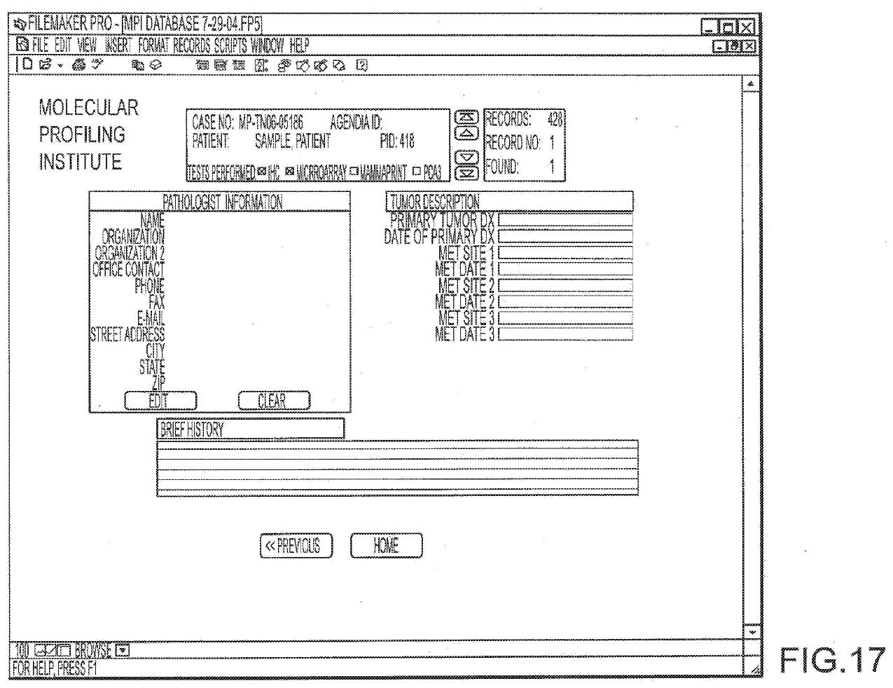

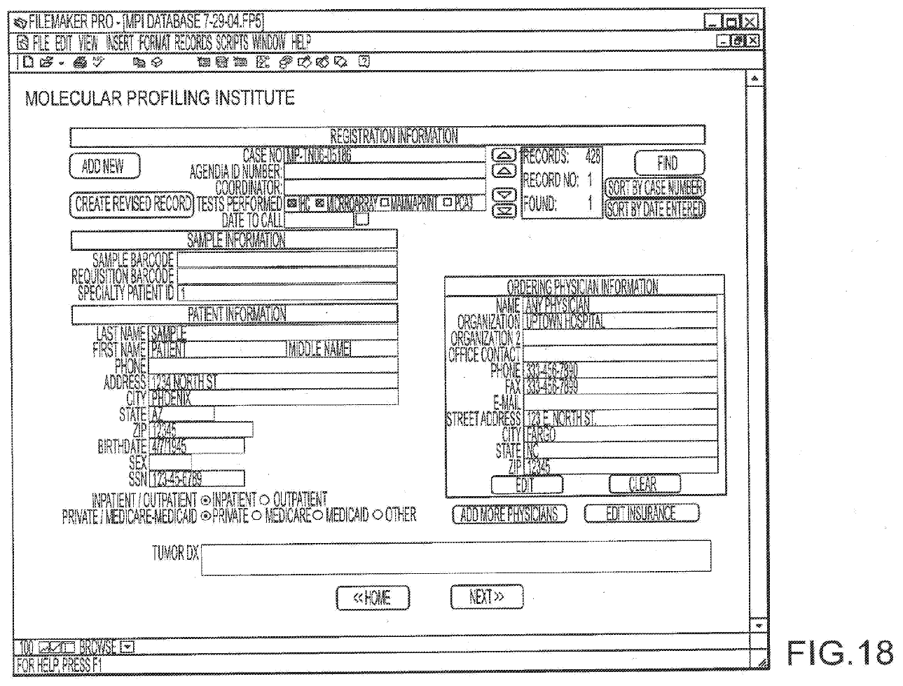

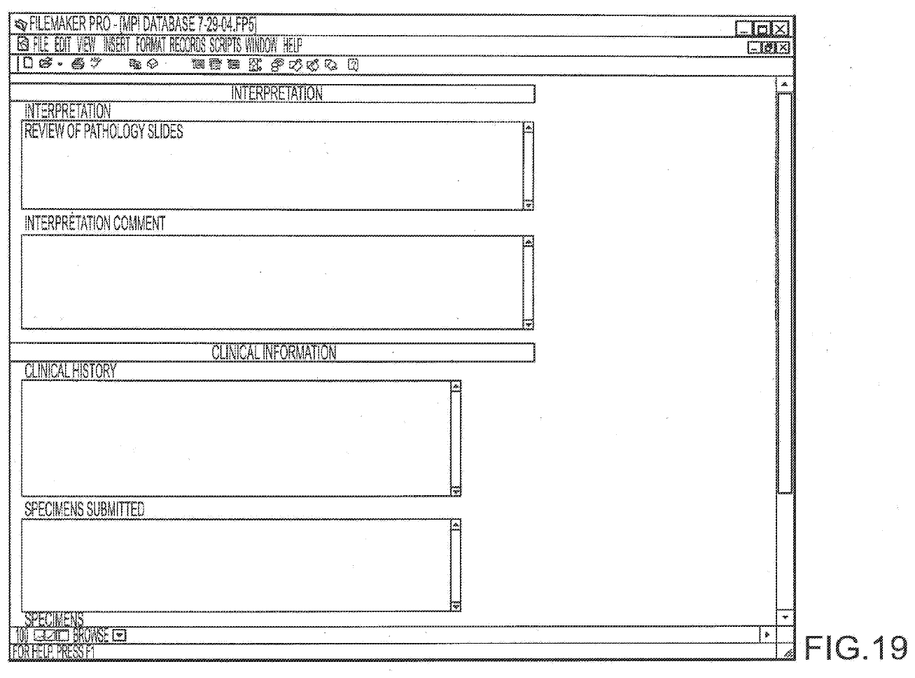

FIGS. 15-25 are computer screen print outs associated with various parts of the information-based personalized medicine drug discovery system and method shown in FIGS. 5-14. FIGS. 15 and 16 show computer screens where physician information and insurance company information is entered on behalf of a client. FIGS. 17-19 show computer screens in which information can be entered for ordering analysis and tests on patient samples.

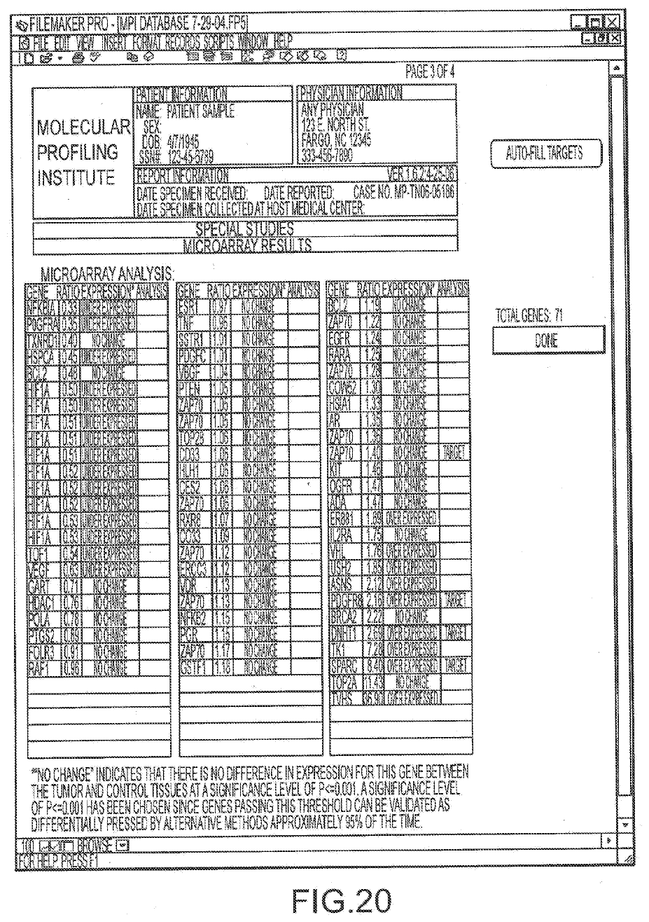

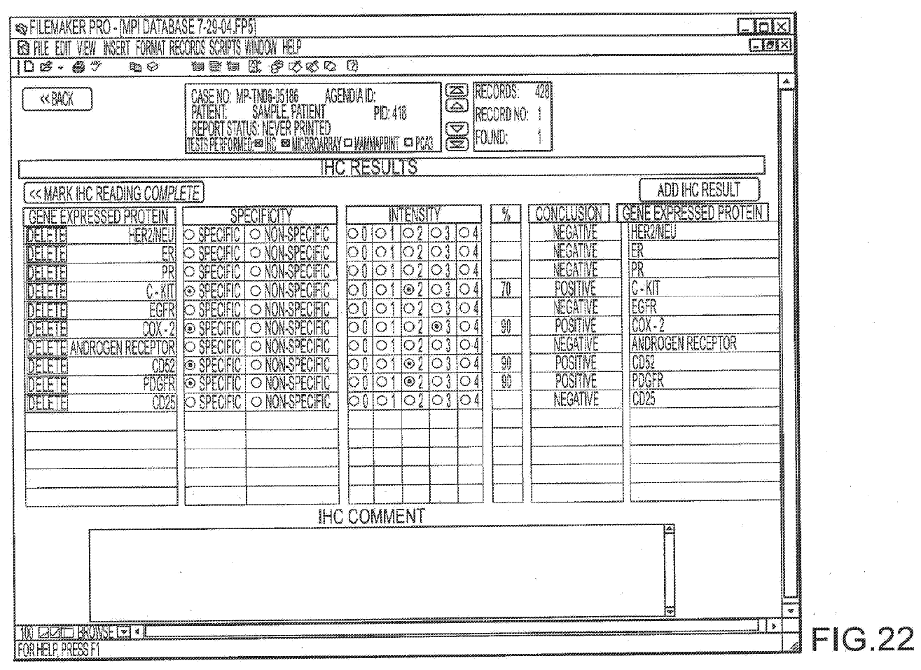

FIG. 20 is a computer screen showing micro array analysis results of specific genes tested with patient samples. This information and computer screen is similar to the information detailed in the patient profile report shown in FIG. 3C. FIG. 22 is a computer screen that shows immunohistochemistry test results for a particular patient for various genes. This information is similar to the information contained in the patient profile report shown in FIG. 3B.

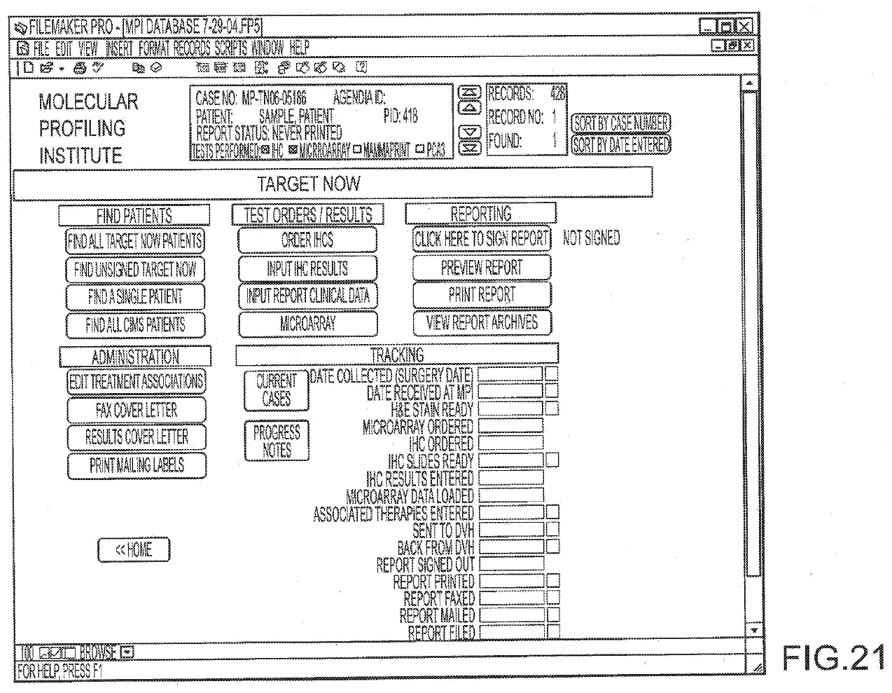

FIG. 21 is a computer screen showing selection options for finding particular patients, ordering tests and/or results, issuing patient reports, and tracking current cases/patients.

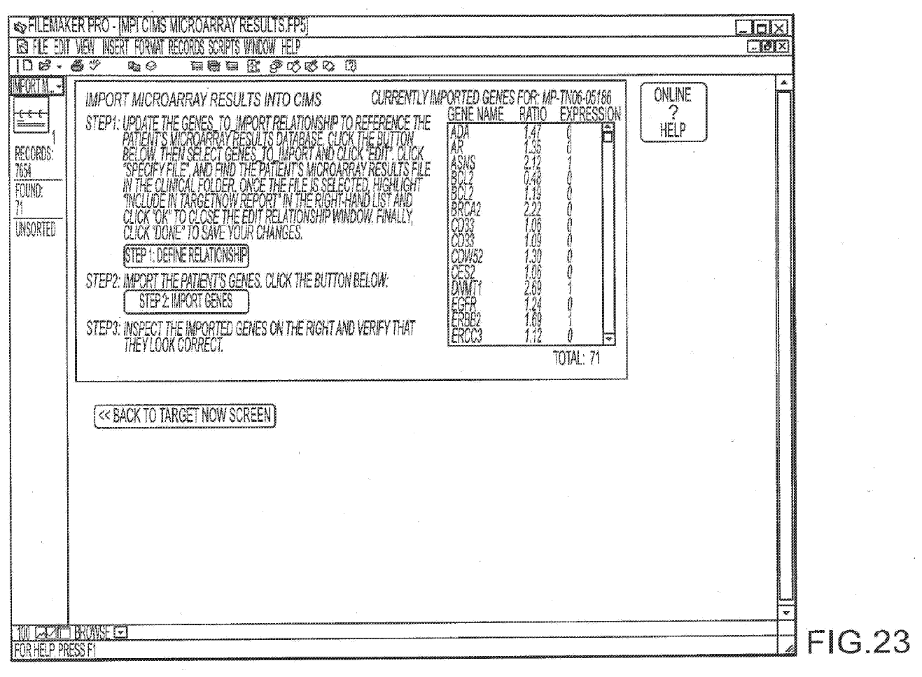

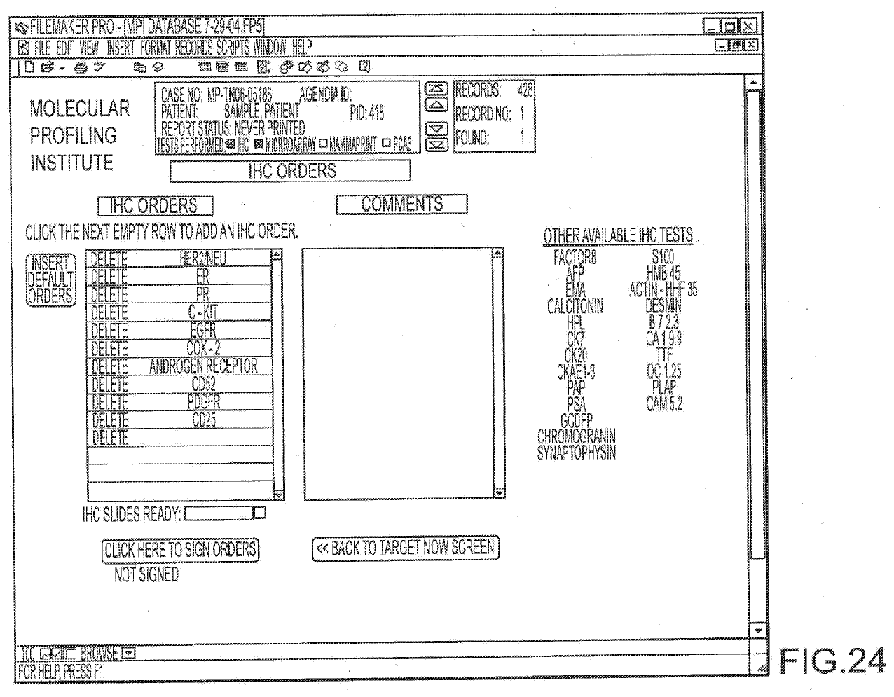

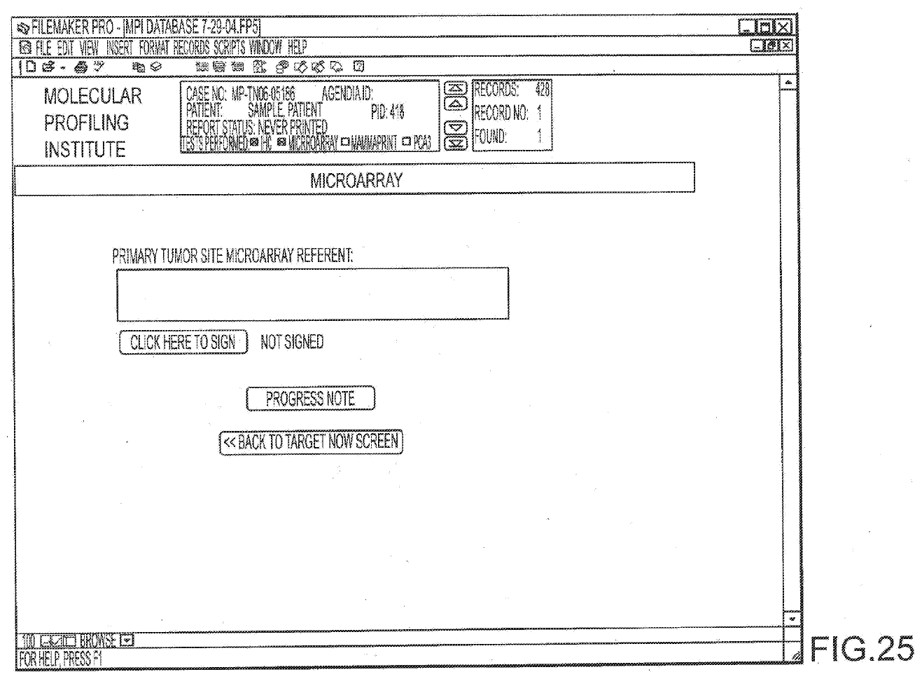

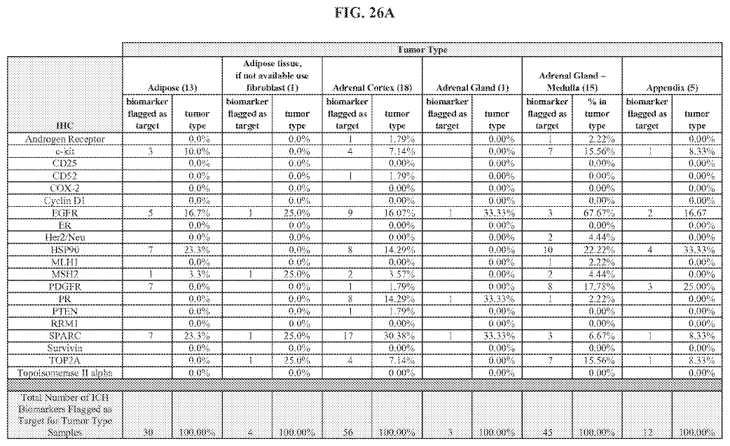

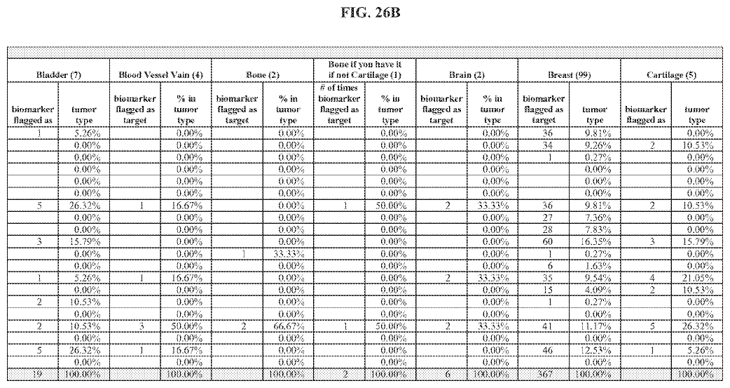

FIG. 23 is a computer screen which outlines some of the steps for creating a patient profile report as shown in FIGS. 3A through 3D. FIG. 24 shows a computer screen for ordering an immunohistochemistry test on a patient sample and FIG. 25 shows a computer screen for entering information regarding a primary tumor site for micro array analysis. It will be understood by those skilled in the art that any number and variety of computer screens may be used to enter the information necessary for using the information-based personalized medicine drug discovery system and method of the present invention and to obtain information resulting from using the information-based personalized medicine drug discovery system and method of the present invention.

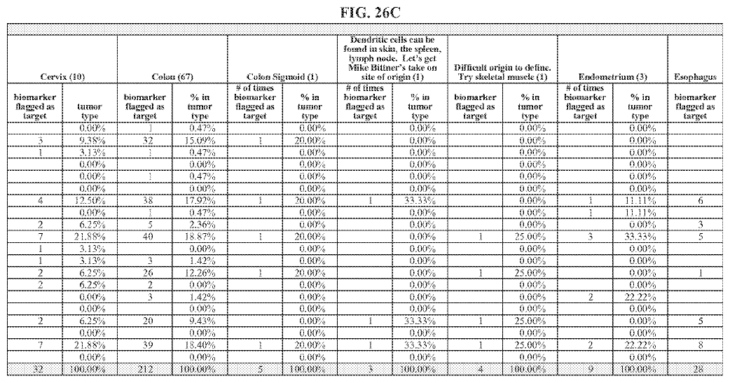

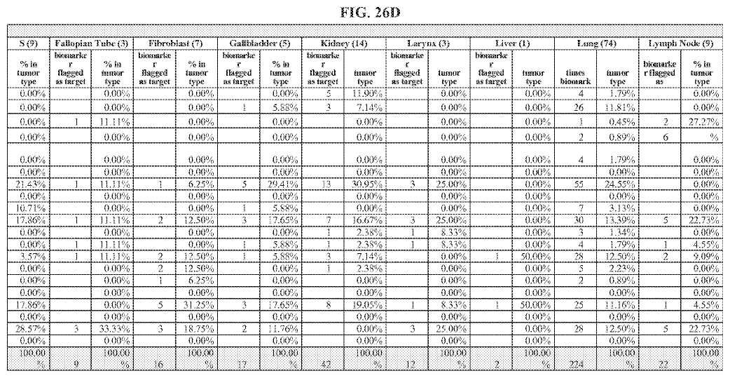

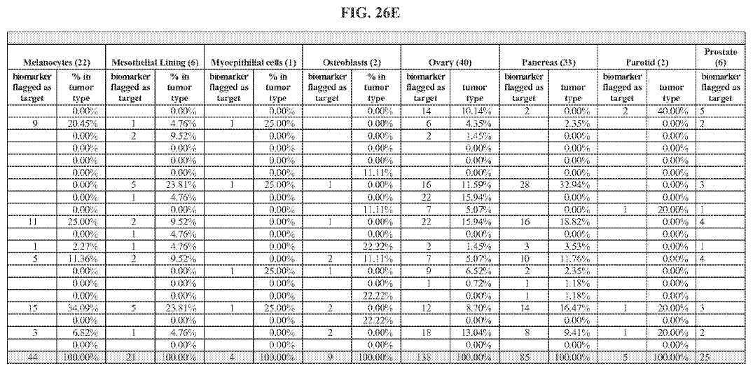

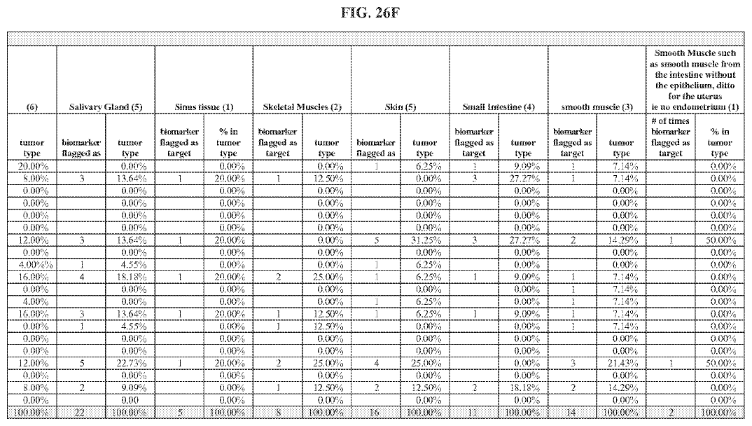

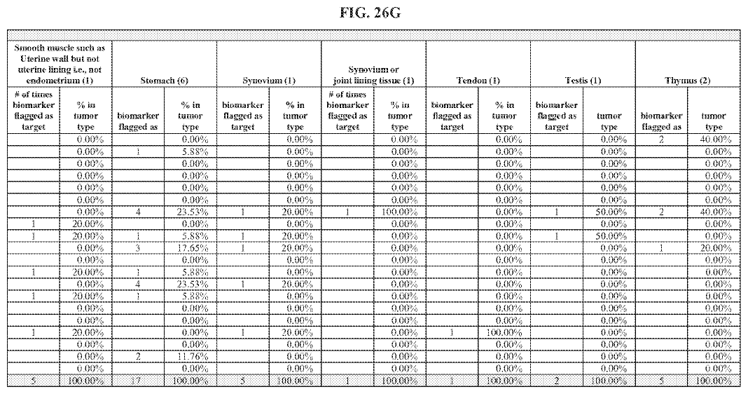

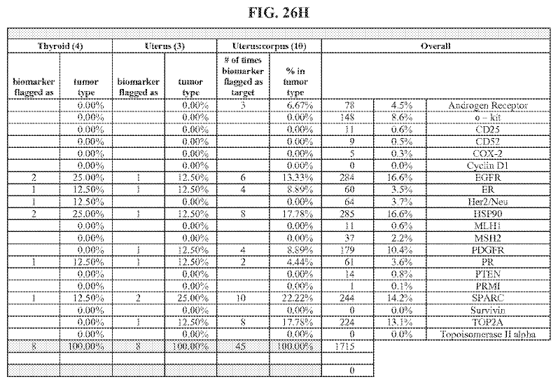

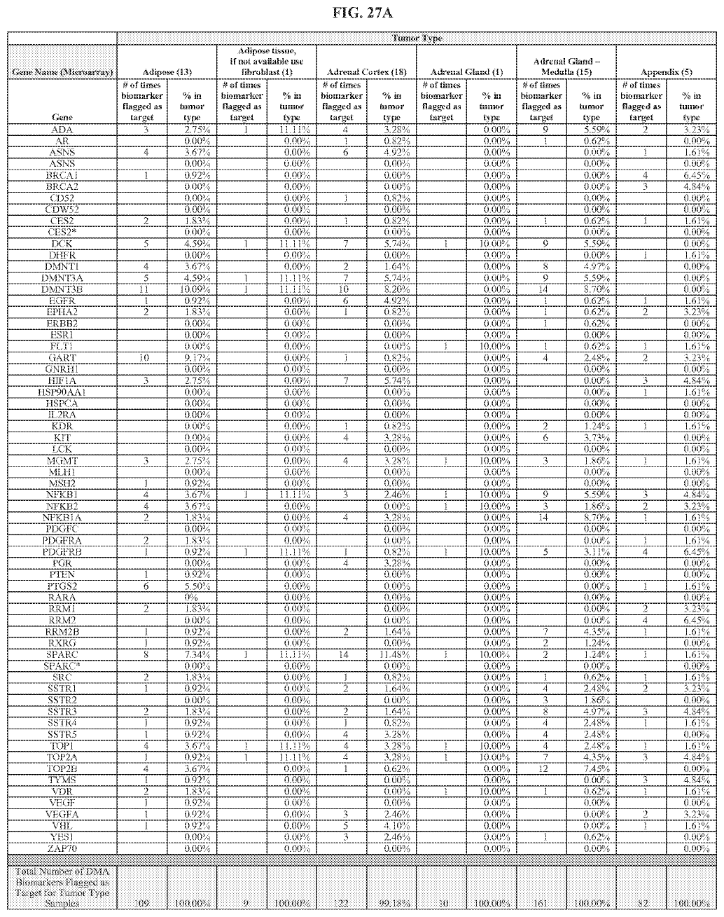

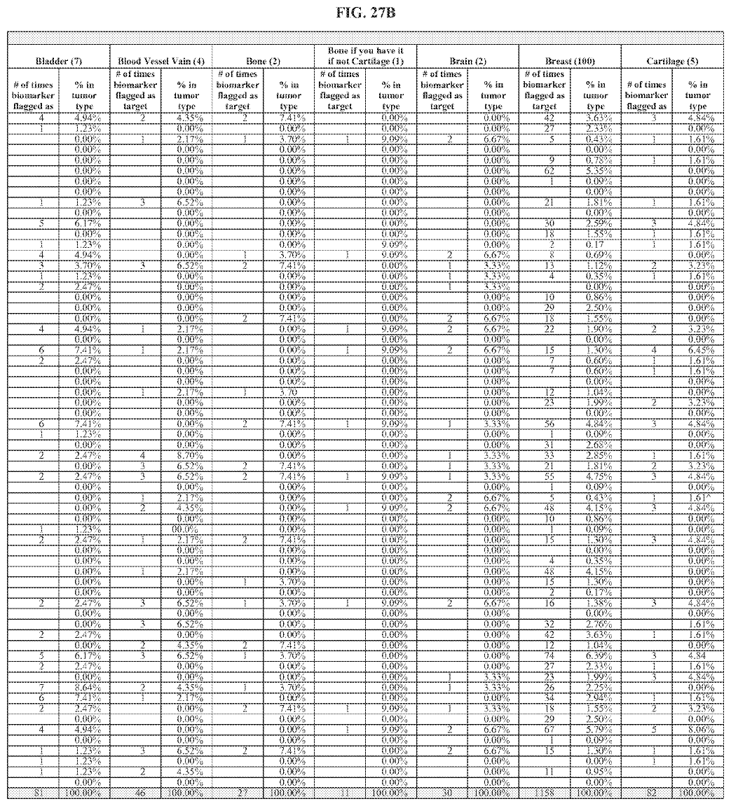

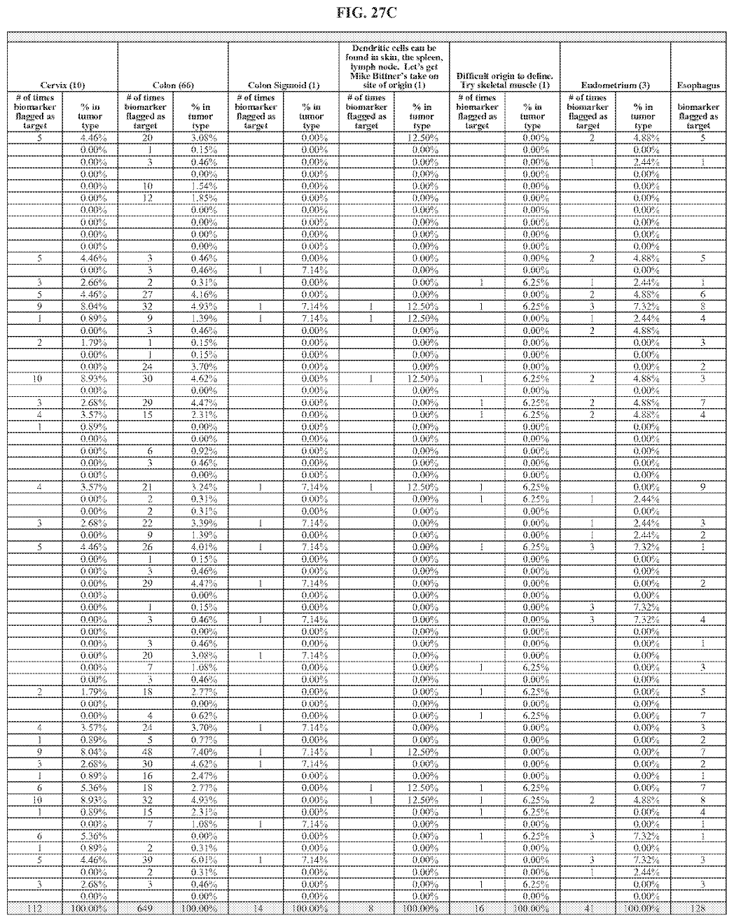

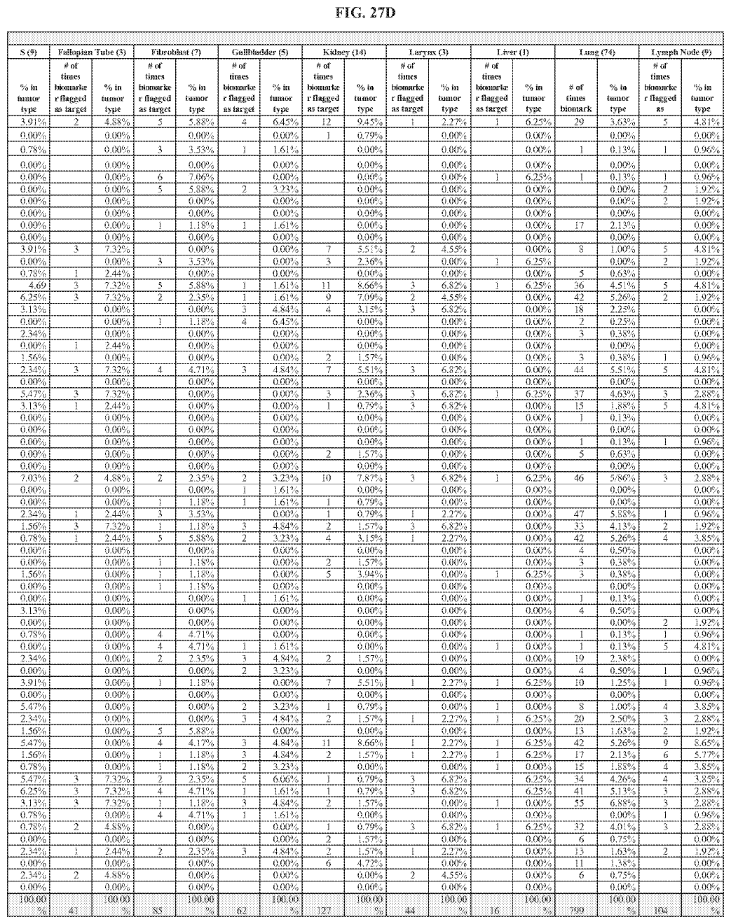

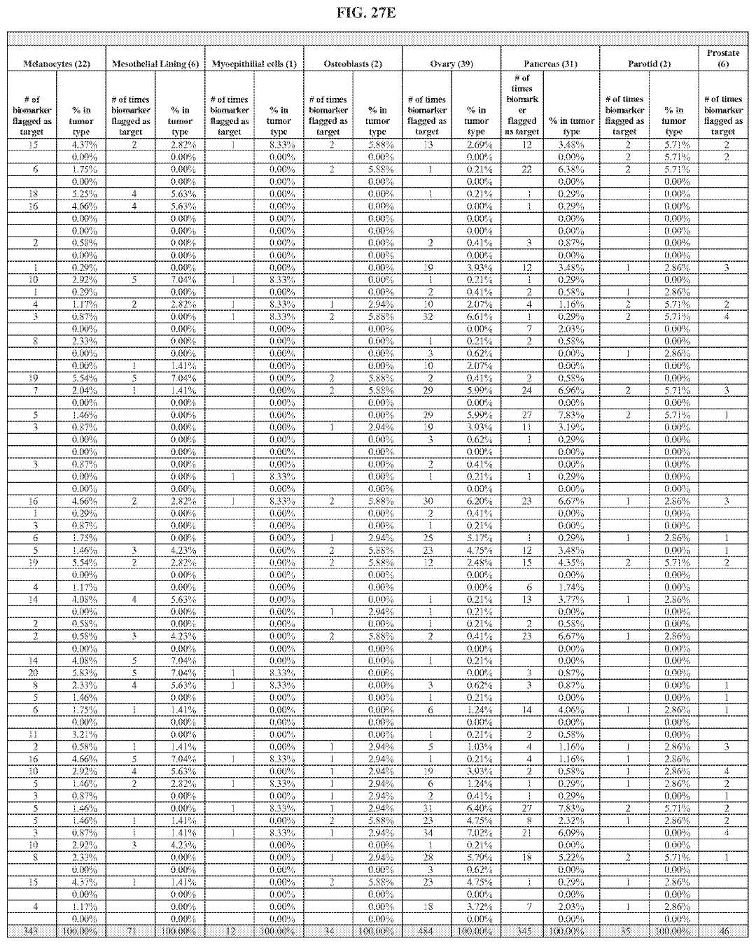

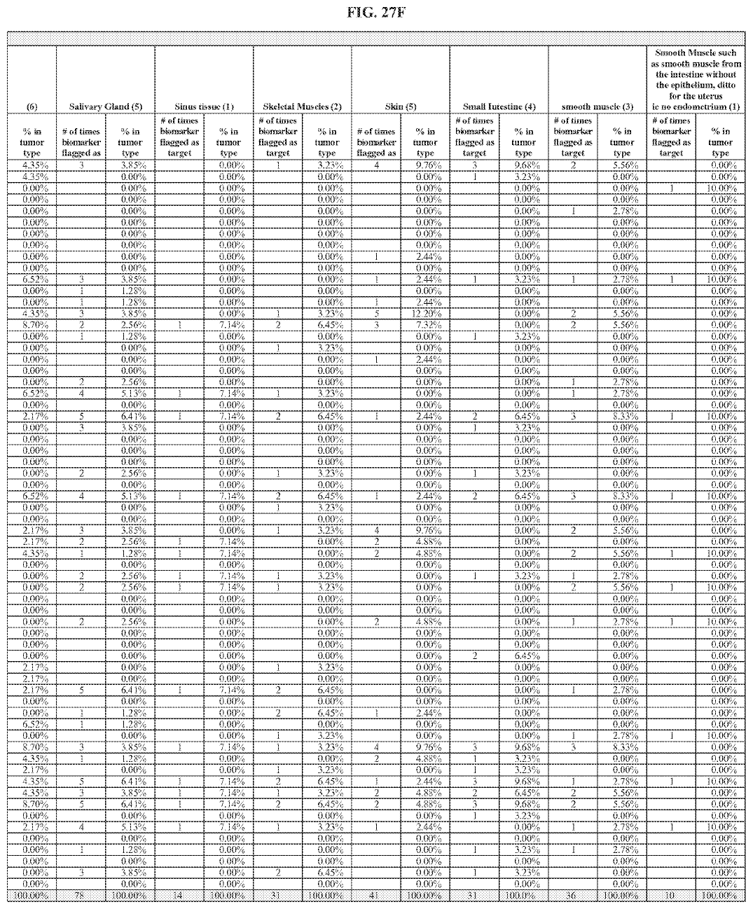

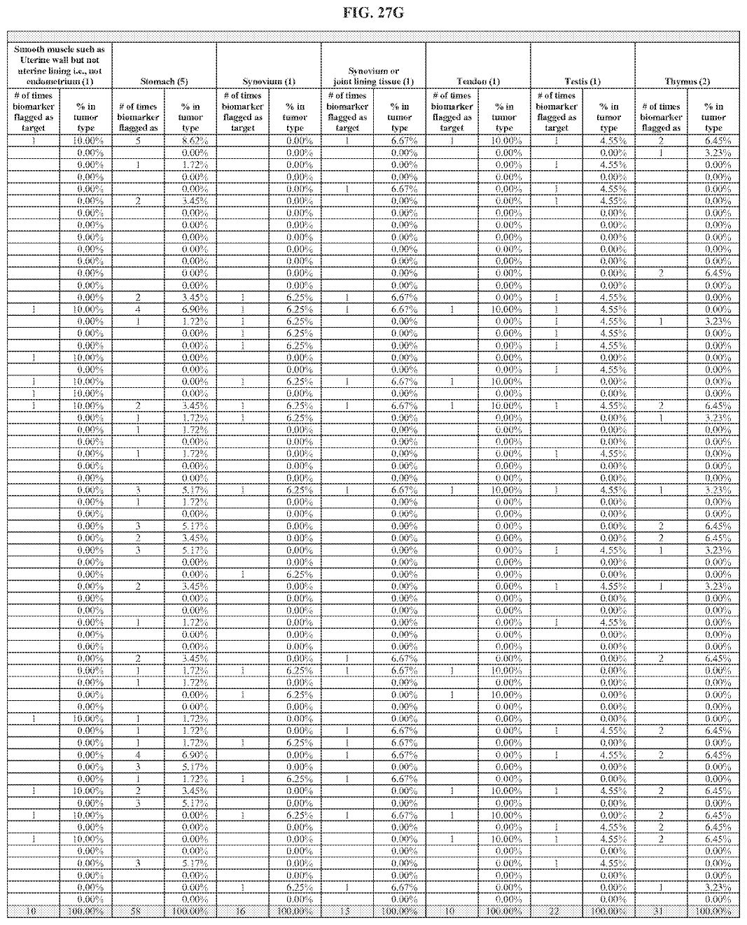

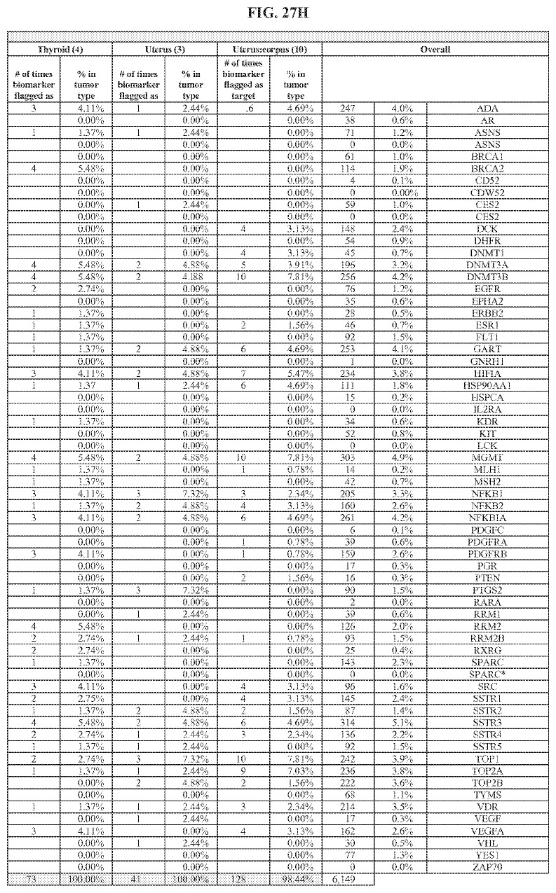

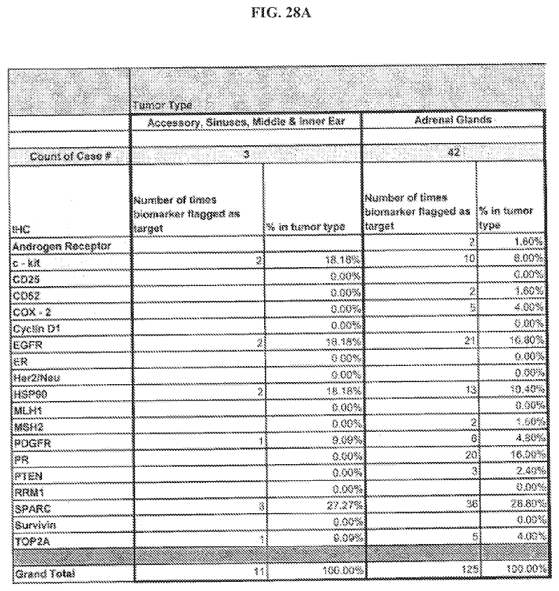

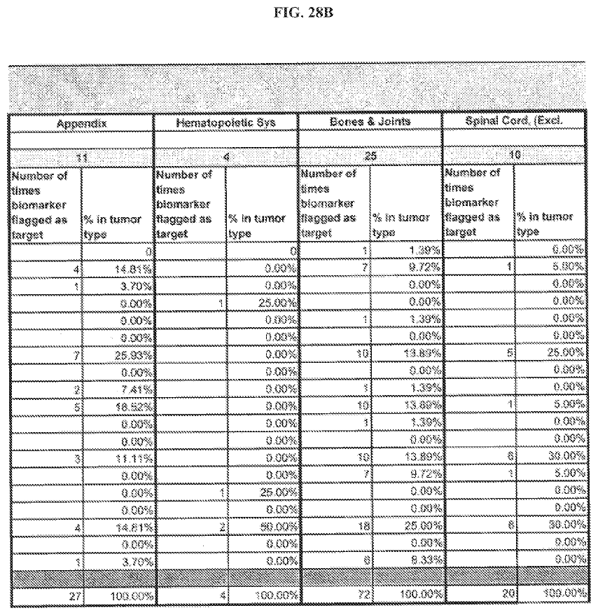

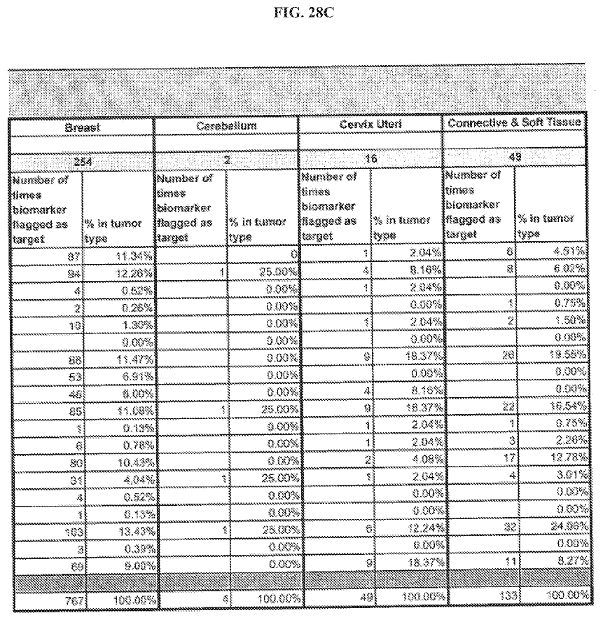

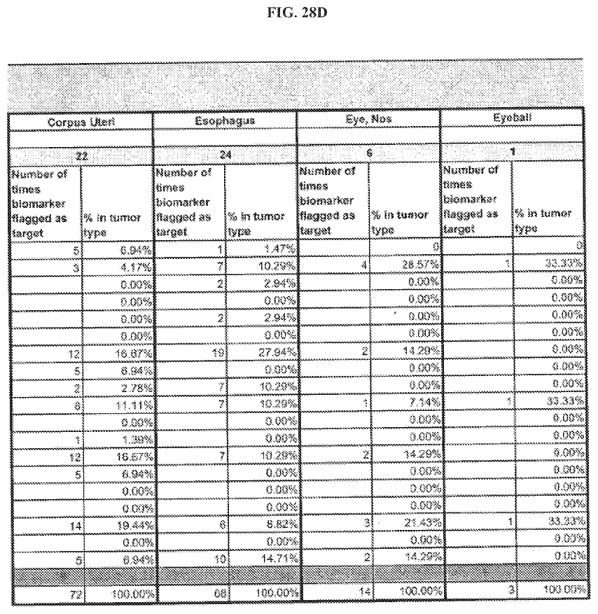

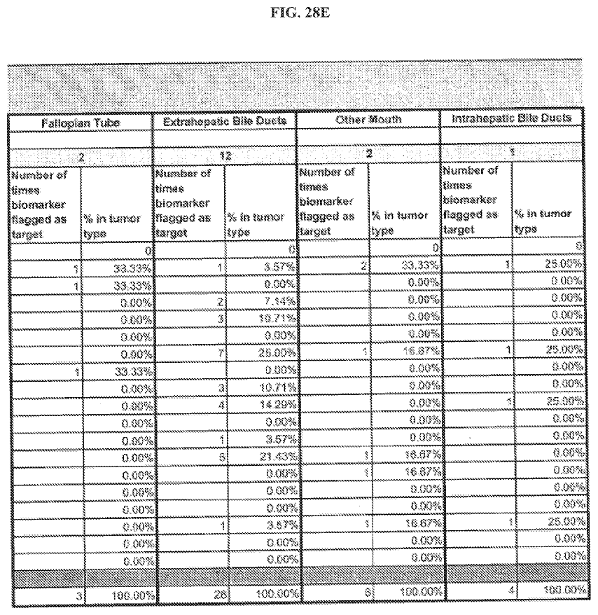

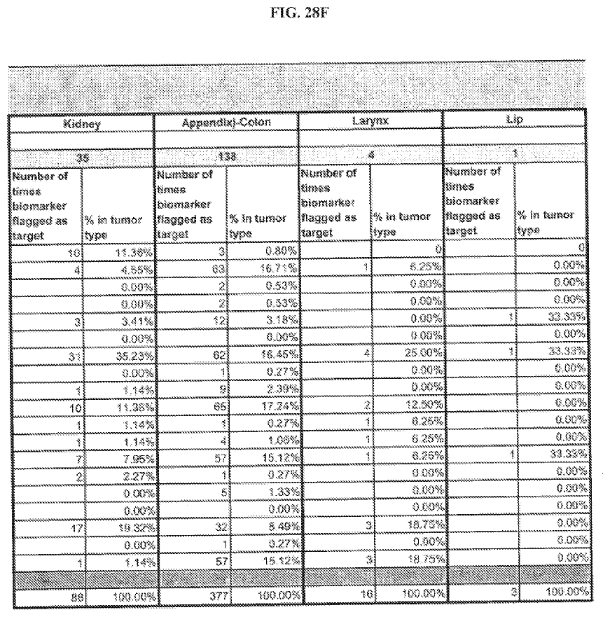

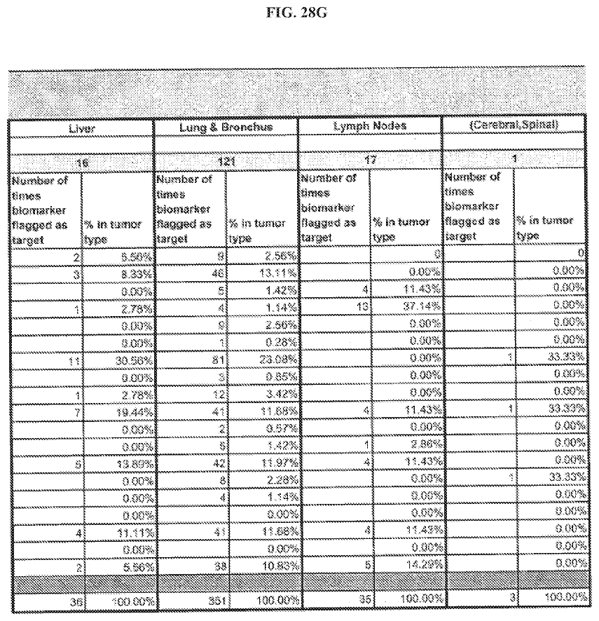

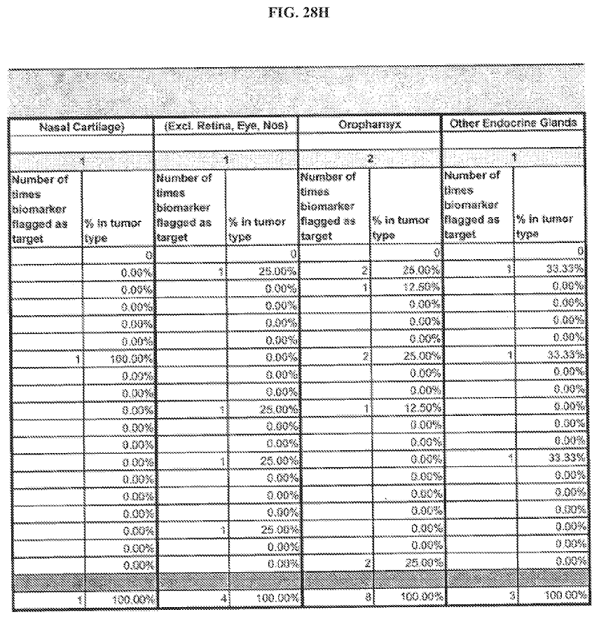

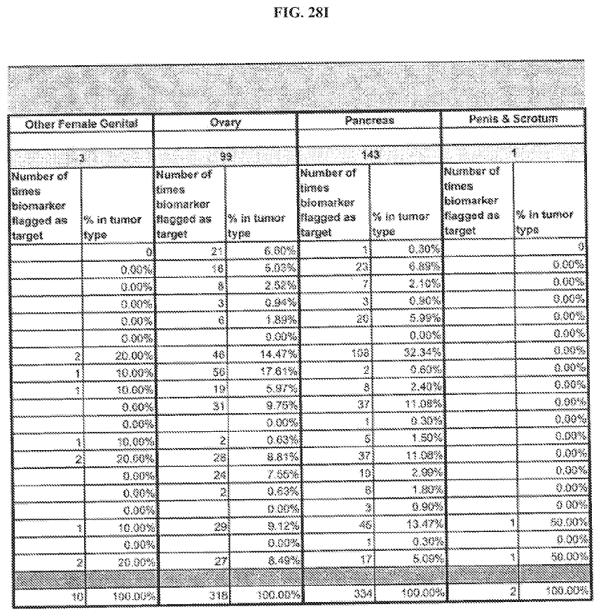

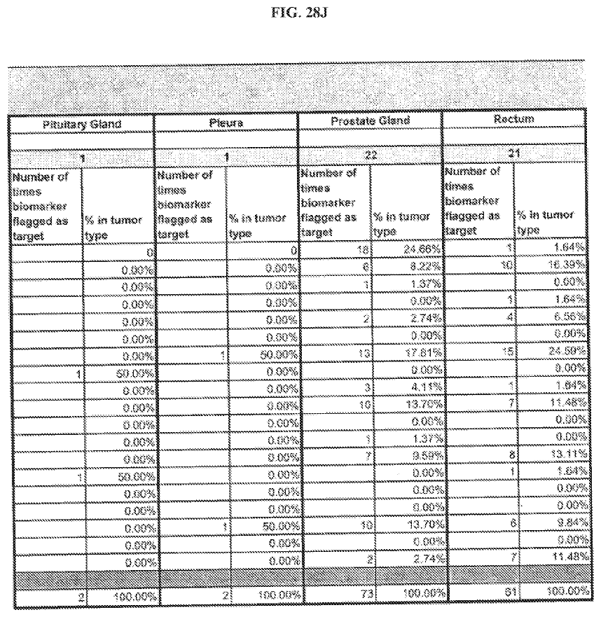

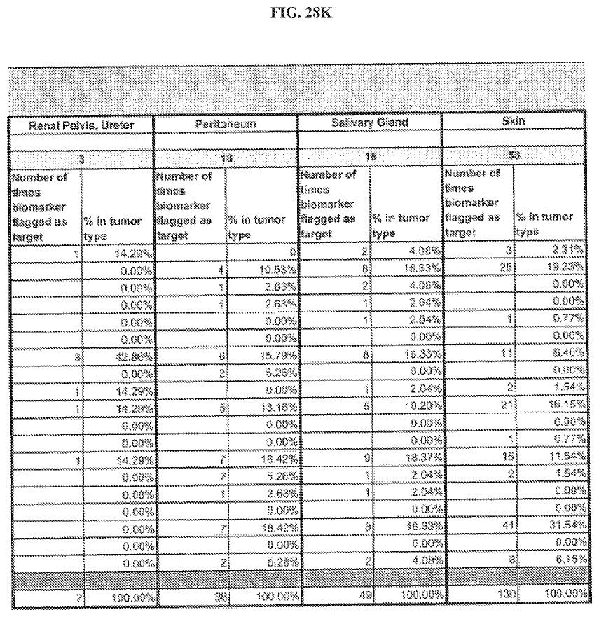

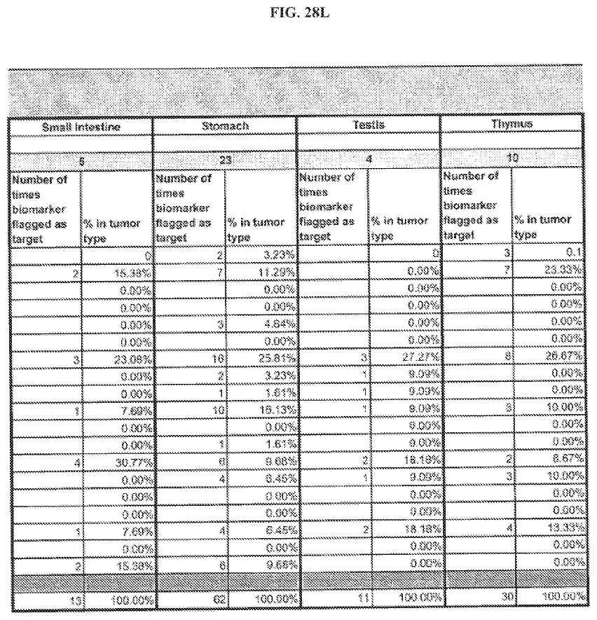

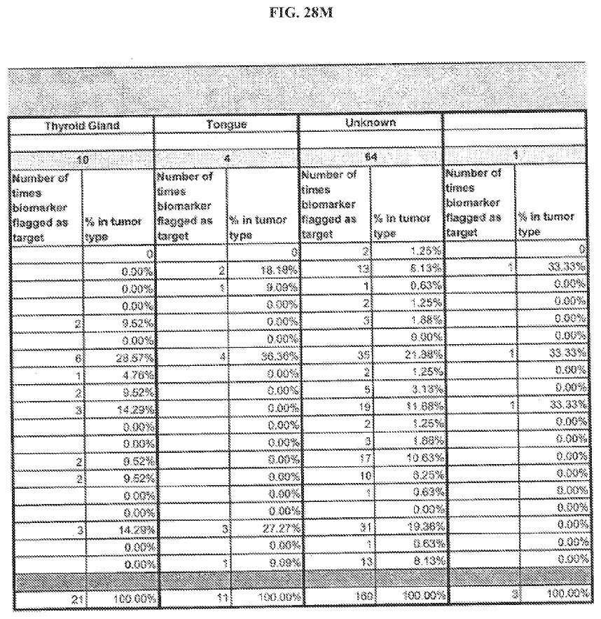

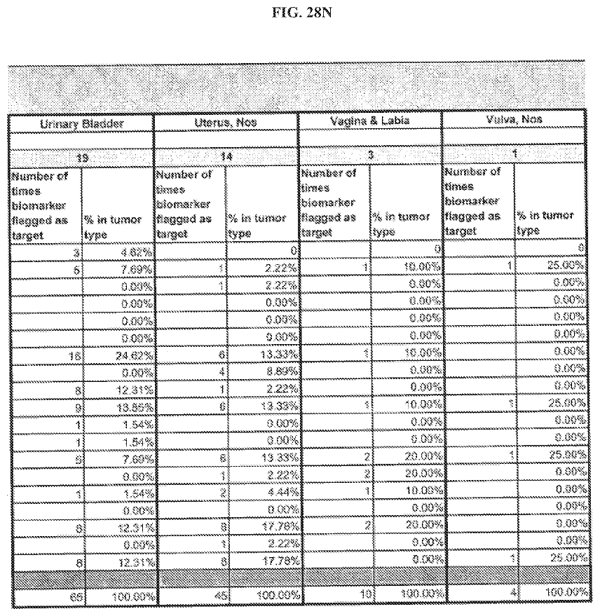

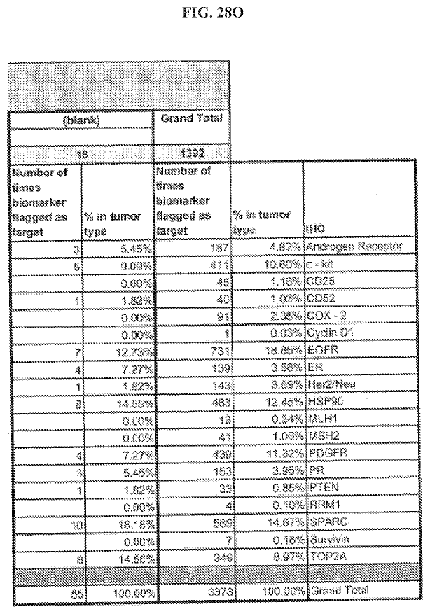

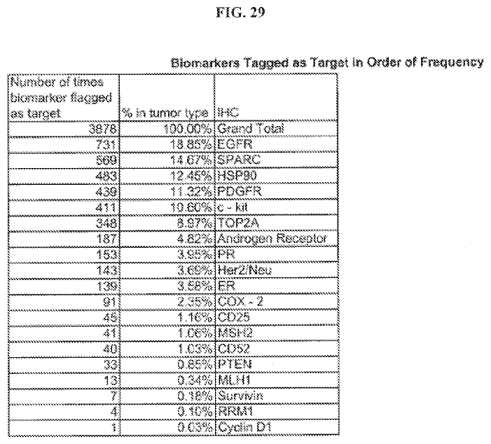

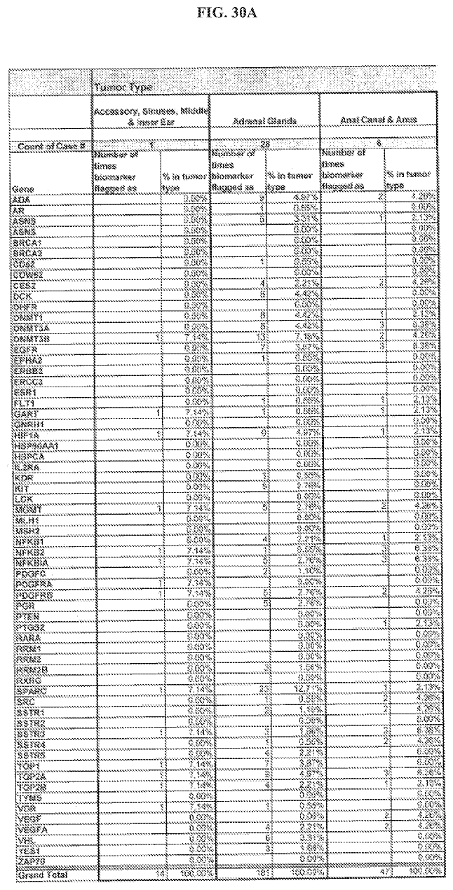

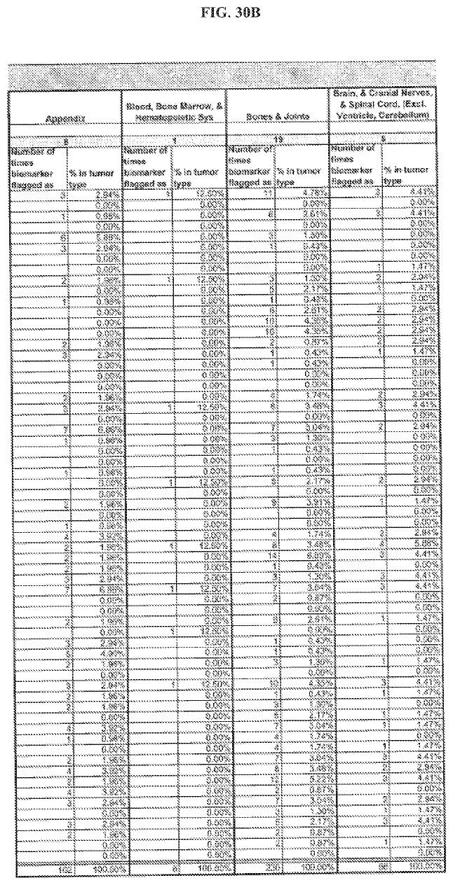

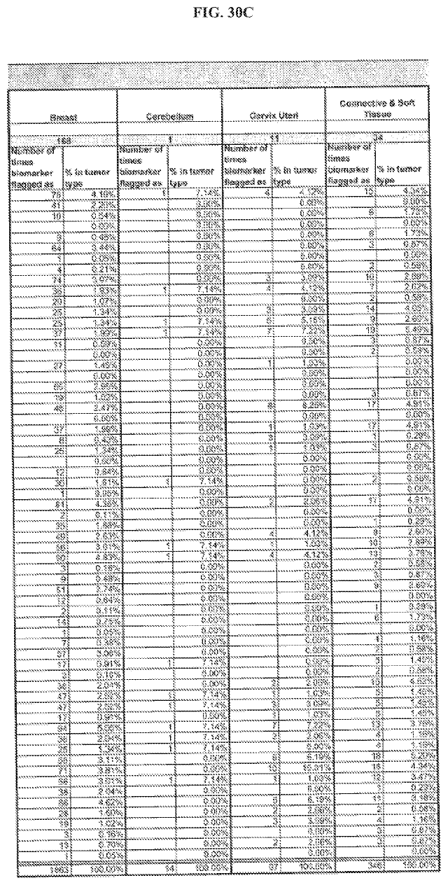

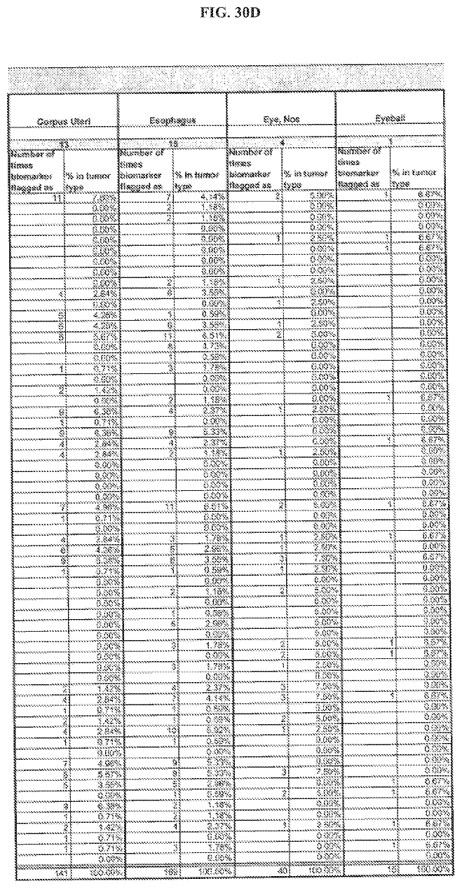

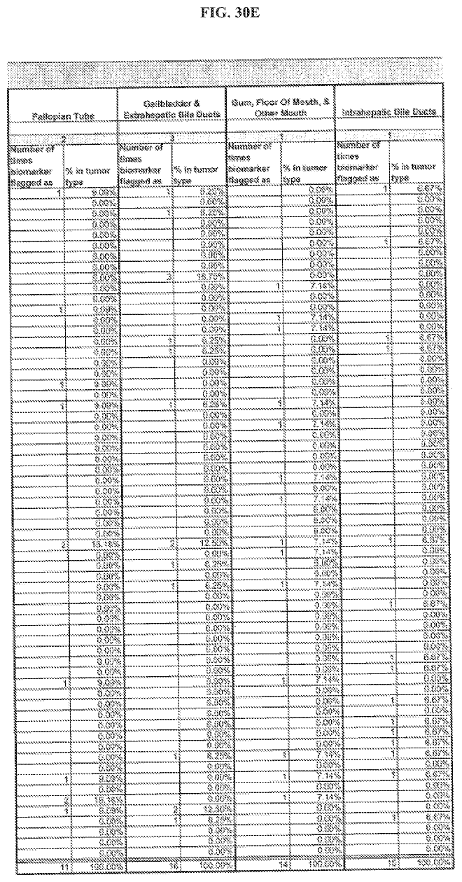

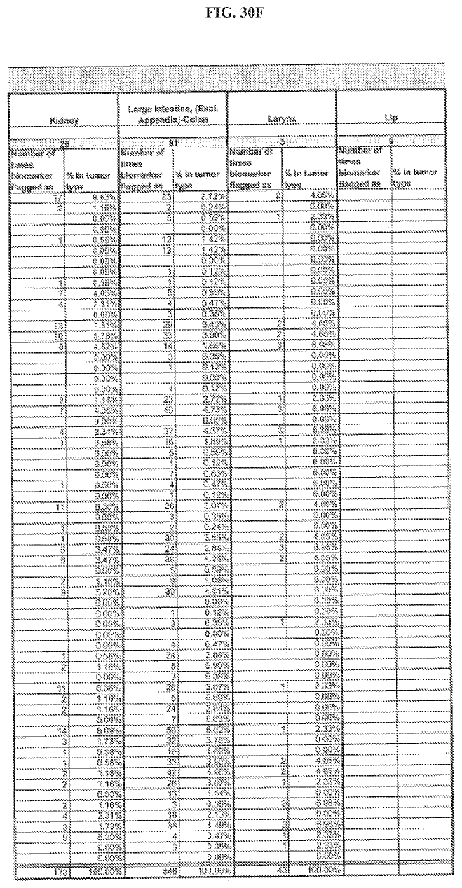

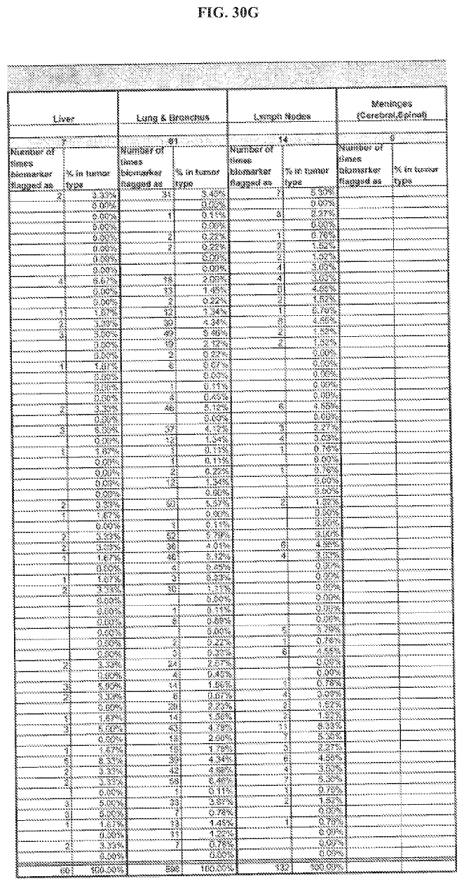

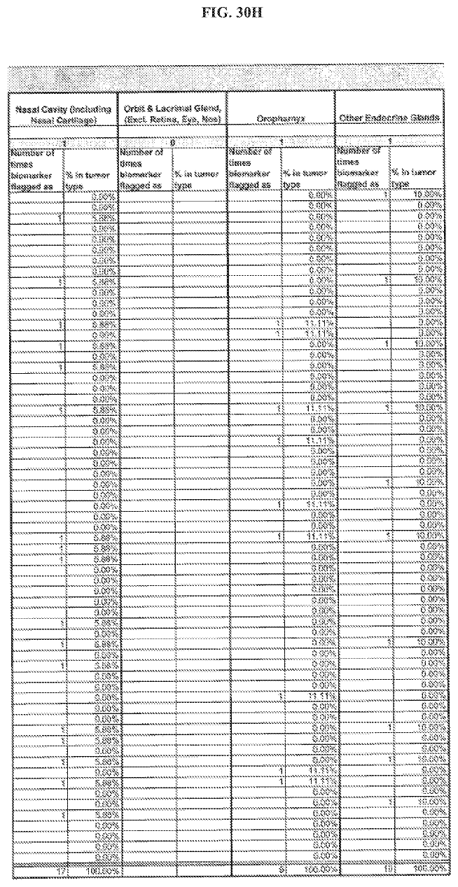

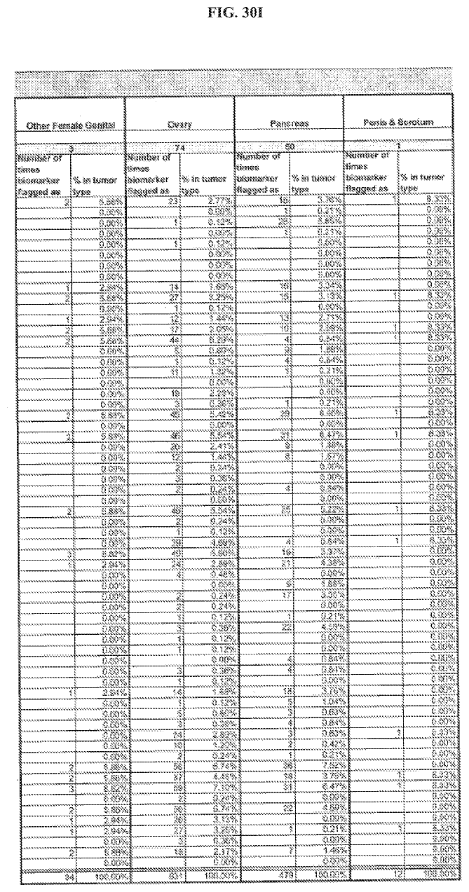

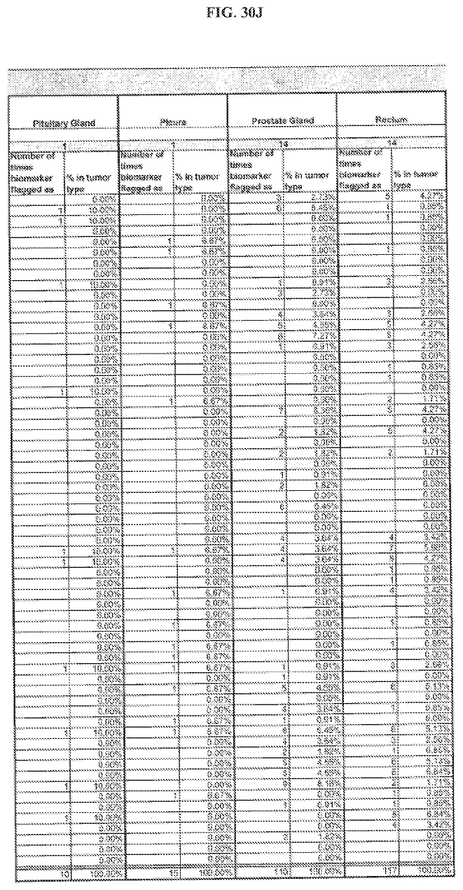

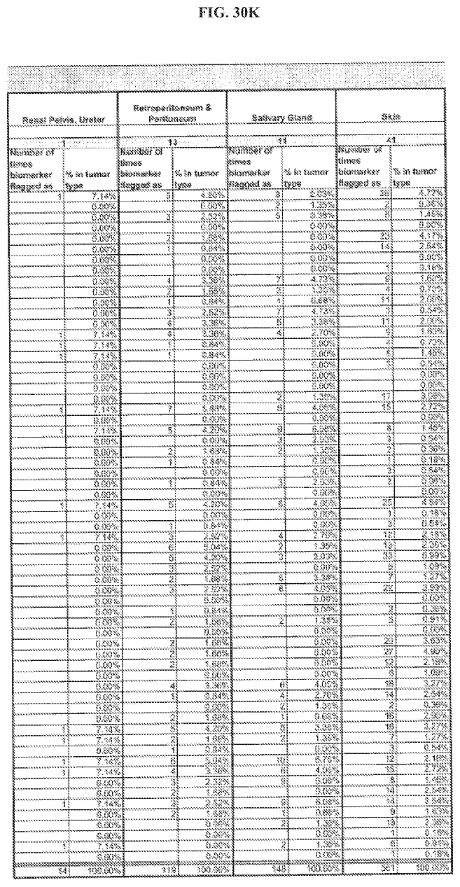

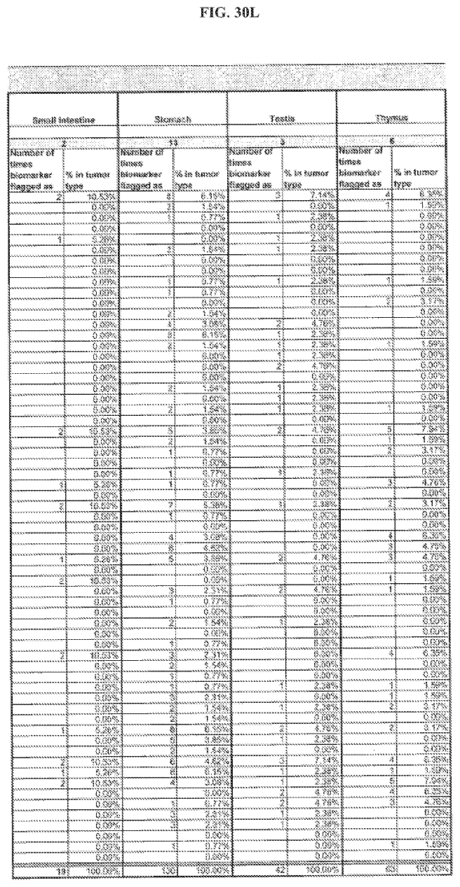

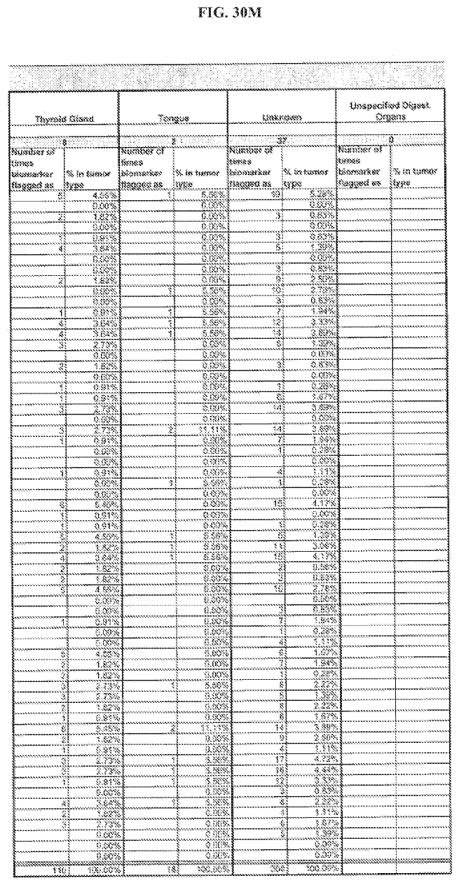

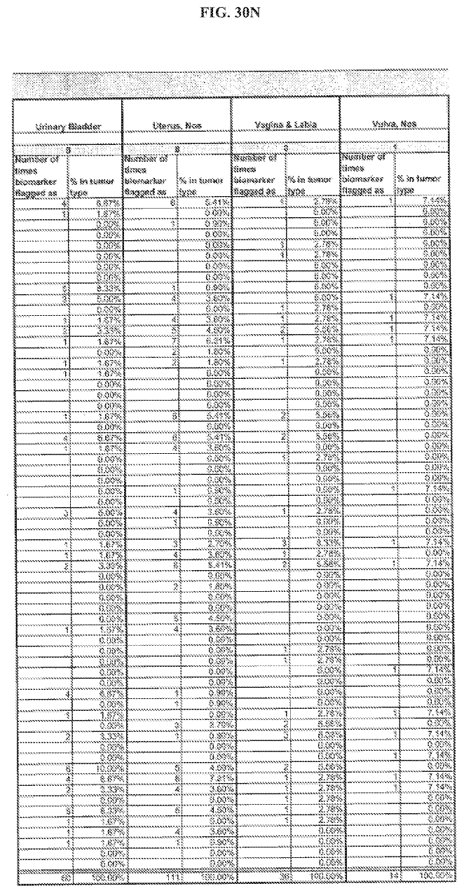

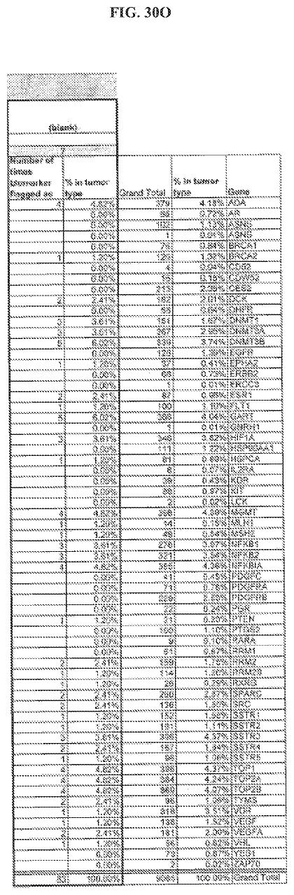

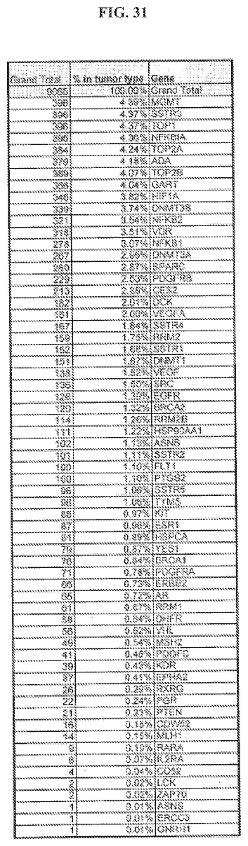

FIGS. 26-31 represent tables that show the frequency of a significant change in expression of certain genes and/or gene expressed proteins by tumor type, i.e. the number of times that a gene and/or gene expressed protein was flagged as a target by tumor type as being significantly overexpressed or underexpressed. The tables show the total number of times a gene and/or gene expressed protein was overexpressed or underexpressed in a particular tumor type and whether the change in expression was determined by immunohistochemistry analysis (FIG. 26, FIG. 28) or gene expression analysis (FIGS. 27, 30). The tables also identify the total number of times an overexpression of any gene expressed protein occurred in a particular tumor type using immunohistochemistry and the total number of times an overexpression or underexpression of any gene occurred in a particular tumor type using gene microarray analysis.

The systems of the invention can be used to automate the steps of identifying a molecular profile to assess a cancer. In an aspect, the invention provides a method of generating a report comprising a molecular profile. The method comprises: performing a search on an electronic medium to obtain a data set, wherein the data set comprises a plurality of scientific publications corresponding to plurality of cancer biomarkers; and analyzing the data set to identify a rule set linking a characteristic of each of the plurality of cancer biomarkers with an expected benefit of a plurality of treatment options, thereby identifying the cancer biomarkers included within a molecular profile. The method can further comprise performing molecular profiling on a sample from a subject to assess the characteristic of each of the plurality of cancer biomarkers, and compiling a report comprising the assessed characteristics into a list, thereby generating a report that identifies a molecular profile for the sample. The report can further comprise a list describing the expected benefit of the plurality of treatment options based on the assessed characteristics, thereby identifying candidate treatment options for the subject. The sample from the subject may comprise cancer cells. The cancer can be any cancer disclosed herein or known in the art.

The characteristic of each of the plurality of cancer biomarkers can be any useful characteristic for molecular profiling as disclosed herein or known in the art. Such characteristics include without limitation mutations (point mutations, insertions, deletions, rearrangements, etc), epigenetic modifications, copy number, nucleic acid or protein expression levels, post-translational modifications, and the like.

In an embodiment, the method further comprises identifying a priority list as amongst said plurality of cancer biomarkers. The priority list can be sorted according to any appropriate priority criteria. In an embodiment, the priority list is sorted according to strength of evidence in the plurality of scientific publications linking the cancer biomarkers to the expected benefit. In another embodiment, the priority list is sorted according to strength of the expected benefit. In still another embodiment, the priority list is sorted according to strength of the expected benefit. One of skill will appreciate that the priority list can be sorted according to a combination of these or other appropriate priority criteria. The candidate treatment options can be sorted according to the priority list, thereby identifying a ranked list of treatment options for the subject.

The candidate treatment options can be categorized by expected benefit to the subject. For example, the candidate treatment options can categorized as those that are expected to provide benefit, those that are not expected to provide benefit, or those whose expected benefit cannot be determined.

The candidate treatment options can include regulatory approved and/or on-compendium treatments for the cancer. The candidate treatment options can include regulatory approved but off-label treatments for the cancer, such as a treatment that has been approved for a cancer of another lineage. The candidate treatment options can include treatments that are under development, such as in ongoing clinical trials. The report may identify treatments as approved, on- or off-compendium, in clinical trials, and the like.

In some embodiments, the method further comprises analyzing the data set to select a laboratory technique to assess the characteristics of the biomarkers, thereby designating a technique that can be used to assess the characteristic for each of the plurality of biomarkers. In other embodiments, the laboratory technique is chosen based on its applicability to assess the characteristic of each of the biomarkers. The laboratory techniques can be those disclosed herein, including without limitation FISH for gene copy number or mutation analysis, IHC for protein expression levels, RT-PCR for mutation or expression analysis, sequencing or fragment analysis for mutation analysis. Sequencing includes any useful sequencing method disclosed herein or known in the art, including without limitation Sanger sequencing, pyrosequencing, or next generation sequencing methods.

In a related aspect, the invention provides a method comprising: performing a search on an electronic medium to obtain a data set comprising a plurality of scientific publications corresponding to plurality of cancer biomarkers; analyzing the data set to select a method to assess a characteristic of each of the cancer biomarkers, thereby designating a method for characterizing each of the biomarkers; further analyzing the data set to select a rule set that identifies a priority list as amongst the biomarkers; performing tumor profiling on a tumor sample from a subject comprising the selected methods to determine the status of the characteristic of each of the biomarkers; and compiling the status in a report according to said priority list; thereby generating a report that identifies a tumor profile.

### Molecular Profiling Targets

The present invention provides methods and systems for analyzing diseased tissue using molecular profiling as previously described above. Because the methods rely on analysis of the characteristics of the tumor under analysis, the methods can be applied in for any tumor or any stage of disease, such an advanced stage of disease or a metastatic tumor of unknown origin. As described herein, a tumor or cancer sample is analyzed for molecular characteristics in order to predict or identify a candidate therapeutic treatment. The molecular characteristics can include the expression of genes or gene products, assessment of gene copy number, or mutational analysis. Any relevant determinable characteristic that can assist in prediction or identification of a candidate therapeutic can be included within the methods of the invention.

The biomarker patterns or biomarker signature sets can be determined for tumor types, diseased tissue types, or diseased cells including without limitation adipose, adrenal cortex, adrenal gland, adrenal gland-medulla, appendix, bladder, blood vessel, bone, bone cartilage, brain, breast, cartilage, cervix, colon, colon sigmoid, dendritic cells, skeletal muscle, endometrium, esophagus, fallopian tube, fibroblast, gallbladder, kidney, larynx, liver, lung, lymph node, melanocytes, mesothelial lining, myoepithelial cells, osteoblasts, ovary, pancreas, parotid, prostate, salivary gland, sinus tissue, skeletal muscle, skin, small intestine, smooth muscle, stomach, synovium, joint lining tissue, tendon, testis, thymus, thyroid, uterus, and uterus corpus.

The methods of the present invention can be used for selecting a treatment of any cancer or tumor type, including but not limited to breast cancer (including HER2+ breast cancer, HER2− breast cancer, ER/PR+, HER2− breast cancer, or triple negative breast cancer), pancreatic cancer, cancer of the colon and/or rectum, leukemia, skin cancer, bone cancer, prostate cancer, liver cancer, lung cancer, brain cancer, cancer of the larynx, gallbladder, parathyroid, thyroid, adrenal, neural tissue, head and neck, stomach, bronchi, kidneys, basal cell carcinoma, squamous cell carcinoma of both ulcerating and papillary type, metastatic skin carcinoma, osteo sarcoma, Ewing's sarcoma, veticulum cell sarcoma, myeloma, giant cell tumor, small-cell lung tumor, islet cell carcinoma, primary brain tumor, acute and chronic lymphocytic and granulocytic tumors, hairy-cell tumor, adenoma, hyperplasia, medullary carcinoma, pheochromocytoma, mucosal neuroma, intestinal ganglioneuroma, hyperplastic corneal nerve tumor, marfanoid habitus tumor, Wilm's tumor, seminoma, ovarian tumor, leiomyoma, cervical dysplasia and in situ carcinoma, neuroblastoma, retinoblastoma, soft tissue sarcoma, malignant carcinoid, topical skin lesion, mycosis fungoides, rhabdomyosarcoma, Kaposi's sarcoma, osteogenic and other sarcoma, malignant hypercalcemia, renal cell tumor, polycythermia vera, adenocarcinoma, glioblastoma multiforma, leukemias, lymphomas, malignant melanomas, and epidermoid carcinomas. The cancer or tumor can comprise, without limitation, a carcinoma, a sarcoma, a lymphoma or leukemia, a germ cell tumor, a blastoma, or other cancers. Carcinomas that can be assessed using the subject methods include without limitation epithelial neoplasms, squamous cell neoplasms, squamous cell carcinoma, basal cell neoplasms basal cell carcinoma, transitional cell papillomas and carcinomas, adenomas and adenocarcinomas (glands), adenoma, adenocarcinoma, linitis plastica insulinoma, glucagonoma, gastrinoma, vipoma, cholangiocarcinoma, hepatocellular carcinoma, adenoid cystic carcinoma, carcinoid tumor of appendix, prolactinoma, oncocytoma, hurthle cell adenoma, renal cell carcinoma, grawitz tumor, multiple endocrine adenomas, endometrioid adenoma, adnexal and skin appendage neoplasms, mucoepidermoid neoplasms, cystic, mucinous and serous neoplasms, cystadenoma, pseudomyxoma peritonei, ductal, lobular and medullary neoplasms, acinar cell neoplasms, complex epithelial neoplasms, warthin's tumor, thymoma, specialized gonadal neoplasms, sex cord stromal tumor, thecoma, granulosa cell tumor, arrhenoblastoma, sertoli leydig cell tumor, glomus tumors, paraganglioma, pheochromocytoma, glomus tumor, nevi and melanomas, melanocytic nevus, malignant melanoma, melanoma, nodular melanoma, dysplastic nevus, lentigo maligna melanoma, superficial spreading melanoma, and malignant acral lentiginous melanoma. Sarcoma that can be assessed using the subject methods include without limitation Askin's tumor, botryodies, chondrosarcoma, Ewing's sarcoma, malignant hemangio endothelioma, malignant schwannoma, osteosarcoma, soft tissue sarcomas including: alveolar soft part sarcoma, angiosarcoma, cystosarcoma phyllodes, dermatofibrosarcoma, desmoid tumor, desmoplastic small round cell tumor, epithelioid sarcoma, extraskeletal chondrosarcoma, extraskeletal osteosarcoma, fibrosarcoma, hemangiopericytoma, hemangiosarcoma, kaposi's sarcoma, leiomyosarcoma, liposarcoma, lymphangiosarcoma, lymphosarcoma, malignant fibrous histiocytoma, neurofibrosarcoma, rhabdomyosarcoma, and synovialsarcoma. Lymphoma and leukemia that can be assessed using the subject methods include without limitation chronic lymphocytic leukemia/small lymphocytic lymphoma, B-cell prolymphocytic leukemia, lymphoplasmacytic lymphoma (such as waldenstrom macroglobulinemia), splenic marginal zone lymphoma, plasma cell myeloma, plasmacytoma, monoclonal immunoglobulin deposition diseases, heavy chain diseases, extranodal marginal zone B cell lymphoma, also called malt lymphoma, nodal marginal zone B cell lymphoma (nmzl), follicular lymphoma, mantle cell lymphoma, diffuse large B cell lymphoma, mediastinal (thymic) large B cell lymphoma, intravascular large B cell lymphoma, primary effusion lymphoma, burkitt lymphoma/leukemia, T cell prolymphocytic leukemia, T cell large granular lymphocytic leukemia, aggressive NK cell leukemia, adult T cell leukemia/lymphoma, extranodal NK/T cell lymphoma, nasal type, enteropathy-type T cell lymphoma, hepatosplenic T cell lymphoma, blastic NK cell lymphoma, mycosis fungoides/sezary syndrome, primary cutaneous CD30-positive T cell lymphoproliferative disorders, primary cutaneous anaplastic large cell lymphoma, lymphomatoid papulosis, angioimmunoblastic T cell lymphoma, peripheral T cell lymphoma, unspecified, anaplastic large cell lymphoma, classical Hodgkin lymphomas (nodular sclerosis, mixed cellularity, lymphocyte-rich, lymphocyte depleted or not depleted), and nodular lymphocyte-predominant Hodgkin lymphoma. Germ cell tumors that can be assessed using the subject methods include without limitation germinoma, dysgerminoma, seminoma, nongerminomatous germ cell tumor, embryonal carcinoma, endodermal sinus turmor, choriocarcinoma, teratoma, polyembryoma, and gonadoblastoma. Blastoma includes without limitation nephroblastoma, medulloblastoma, and retinoblastoma. Other cancers include without limitation labial carcinoma, larynx carcinoma, hypopharynx carcinoma, tongue carcinoma, salivary gland carcinoma, gastric carcinoma, adenocarcinoma, thyroid cancer (medullary and papillary thyroid carcinoma), renal carcinoma, kidney parenchyma carcinoma, cervix carcinoma, uterine corpus carcinoma, endometrium carcinoma, chorion carcinoma, testis carcinoma, urinary carcinoma, melanoma, brain tumors such as glioblastoma, astrocytoma, meningioma, medulloblastoma and peripheral neuroectodermal tumors, gall bladder carcinoma, bronchial carcinoma, multiple myeloma, basalioma, teratoma, retinoblastoma, choroidea melanoma, seminoma, rhabdomyosarcoma, craniopharyngeoma, osteosarcoma, chondrosarcoma, myosarcoma, liposarcoma, fibrosarcoma, Ewing sarcoma, and plasmocytoma.

In an embodiment, the cancer may be a acute myeloid leukemia (AML), breast carcinoma, cholangiocarcinoma, colorectal adenocarcinoma, extrahepatic bile duct adenocarcinoma, female genital tract malignancy, gastric adenocarcinoma, gastroesophageal adenocarcinoma, gastrointestinal stromal tumors (GIST), glioblastoma, head and neck squamous carcinoma, leukemia, liver hepatocellular carcinoma, low grade glioma, lung bronchioloalveolar carcinoma (BAC), lung non-small cell lung cancer (NSCLC), lung small cell cancer (SCLC), lymphoma, male genital tract malignancy, malignant solitary fibrous tumor of the pleura (MSFT), melanoma, multiple myeloma, neuroendocrine tumor, nodal diffuse large B-cell lymphoma, non epithelial ovarian cancer (non-EOC), ovarian surface epithelial carcinoma, pancreatic adenocarcinoma, pituitary carcinomas, oligodendroglioma, prostatic adenocarcinoma, retroperitoneal or peritoneal carcinoma, retroperitoneal or peritoneal sarcoma, small intestinal malignancy, soft tissue tumor, thymic carcinoma, thyroid carcinoma, or uveal melanoma.

In a further embodiment, the cancer may be a lung cancer including non-small cell lung cancer and small cell lung cancer (including small cell carcinoma (oat cell cancer), mixed small cell/large cell carcinoma, and combined small cell carcinoma), colon cancer, breast cancer, prostate cancer, liver cancer, pancreas cancer, brain cancer, kidney cancer, ovarian cancer, stomach cancer, skin cancer, bone cancer, gastric cancer, breast cancer, pancreatic cancer, glioma, glioblastoma, hepatocellular carcinoma, papillary renal carcinoma, head and neck squamous cell carcinoma, leukemia, lymphoma, myeloma, or a solid tumor.

In embodiments, the cancer comprises an acute lymphoblastic leukemia; acute myeloid leukemia; adrenocortical carcinoma; AIDS-related cancers; AIDS-related lymphoma; anal cancer; appendix cancer; astrocytomas; atypical teratoid/rhabdoid tumor; basal cell carcinoma; bladder cancer; brain stem glioma; brain tumor (including brain stem glioma, central nervous system atypical teratoid/rhabdoid tumor, central nervous system embryonal tumors, astrocytomas, craniopharyngioma, ependymoblastoma, ependymoma, medulloblastoma, medulloepithelioma, pineal parenchymal tumors of intermediate differentiation, supratentorial primitive neuroectodermal tumors and pineoblastoma); breast cancer; bronchial tumors; Burkitt lymphoma; cancer of unknown primary site; carcinoid tumor; carcinoma of unknown primary site; central nervous system atypical teratoid/rhabdoid tumor; central nervous system embryonal tumors; cervical cancer; childhood cancers; chordoma; chronic lymphocytic leukemia; chronic myelogenous leukemia; chronic myeloproliferative disorders; colon cancer; colorectal cancer; craniopharyngioma; cutaneous T-cell lymphoma; endocrine pancreas islet cell tumors; endometrial cancer; ependymoblastoma; ependymoma; esophageal cancer; esthesioneuroblastoma; Ewing sarcoma; extracranial germ cell tumor; extragonadal germ cell tumor; extrahepatic bile duct cancer; gallbladder cancer; gastric (stomach) cancer; gastrointestinal carcinoid tumor; gastrointestinal stromal cell tumor; gastrointestinal stromal tumor (GIST); gestational trophoblastic tumor; glioma; hairy cell leukemia; head and neck cancer; heart cancer; Hodgkin lymphoma; hypopharyngeal cancer; intraocular melanoma; islet cell tumors; Kaposi sarcoma; kidney cancer; Langerhans cell histiocytosis; laryngeal cancer; lip cancer; liver cancer; malignant fibrous histiocytoma bone cancer; medulloblastoma; medulloepithelioma; melanoma; Merkel cell carcinoma; Merkel cell skin carcinoma; mesothelioma; metastatic squamous neck cancer with occult primary; mouth cancer; multiple endocrine neoplasia syndromes; multiple myeloma; multiple myeloma/plasma cell neoplasm; mycosis fungoides; myelodysplastic syndromes; myeloproliferative neoplasms; nasal cavity cancer; nasopharyngeal cancer; neuroblastoma; Non-Hodgkin lymphoma; nonmelanoma skin cancer; non-small cell lung cancer; oral cancer; oral cavity cancer; oropharyngeal cancer; osteosarcoma; other brain and spinal cord tumors; ovarian cancer; ovarian epithelial cancer; ovarian germ cell tumor; ovarian low malignant potential tumor; pancreatic cancer; papillomatosis; paranasal sinus cancer; parathyroid cancer; pelvic cancer; penile cancer; pharyngeal cancer; pineal parenchymal tumors of intermediate differentiation; pineoblastoma; pituitary tumor; plasma cell neoplasm/multiple myeloma; pleuropulmonary blastoma; primary central nervous system (CNS) lymphoma; primary hepatocellular liver cancer; prostate cancer; rectal cancer; renal cancer; renal cell (kidney) cancer; renal cell cancer; respiratory tract cancer; retinoblastoma; rhabdomyosarcoma; salivary gland cancer; Sezary syndrome; small cell lung cancer; small intestine cancer; soft tissue sarcoma; squamous cell carcinoma; squamous neck cancer; stomach (gastric) cancer; supratentorial primitive neuroectodermal tumors; T-cell lymphoma; testicular cancer; throat cancer; thymic carcinoma; thymoma; thyroid cancer; transitional cell cancer; transitional cell cancer of the renal pelvis and ureter; trophoblastic tumor; ureter cancer; urethral cancer; uterine cancer; uterine sarcoma; vaginal cancer; vulvar cancer; Waldenström macroglobulinemia; or Wilm's tumor.

The methods of the invention can be used to determine biomarker patterns or biomarker signature sets in a number of tumor types, diseased tissue types, or diseased cells including accessory, sinuses, middle and inner ear, adrenal glands, appendix, hematopoietic system, bones and joints, spinal cord, breast, cerebellum, cervix uteri, connective and soft tissue, corpus uteri, esophagus, eye, nose, eyeball, fallopian tube, extrahepatic bile ducts, other mouth, intrahepatic bile ducts, kidney, appendix-colon, larynx, lip, liver, lung and bronchus, lymph nodes, cerebral, spinal, nasal cartilage, excl. retina, eye, nos, oropharynx, other endocrine glands, other female genital, ovary, pancreas, penis and scrotum, pituitary gland, pleura, prostate gland, rectum renal pelvis, ureter, peritonem, salivary gland, skin, small intestine, stomach, testis, thymus, thyroid gland, tongue, unknown, urinary bladder, uterus, nos, vagina & labia, and vulva,nos.

In some embodiments, the molecular profiling methods are used to identify a treatment for a cancer of unknown primary (CUP). Approximately 40,000 CUP cases are reported annually in the US. Most of these are metastatic and/or poorly differentiated tumors. Because molecular profiling can identify a candidate treatment depending only upon the diseased sample, the methods of the invention can be used in the CUP setting. Moreover, molecular profiling can be used to create signatures of known tumors, which can then be used to classify a CUP and identify its origin. In an aspect, the invention provides a method of identifying the origin of a CUP, the method comprising performing molecular profiling on a panel of diseased samples to determine a panel of molecular profiles that correlate with the origin of each diseased sample, performing molecular profiling on a CUP sample, and correlating the molecular profile of the CUP sample with the molecular profiling of the panel of diseased samples, thereby identifying the origin of the CUP sample. The identification of the origin of the CUP sample can be made by matching the molecular profile of the CUP sample with the molecular profiles that correlate most closely from the panel of disease samples. The molecular profiling can use any of the techniques described herein, e.g., IHC, FISH, microarray and sequencing. The diseased samples and CUP samples can be derived from a patient sample, e.g., a biopsy sample, including a fine needle biopsy. In one embodiment, DNA microarray and IHC profiling are performed on the panel of diseased samples, DNA microarray is performed on the CUP samples, and then IHC is performed on the CUP sample for a subset of the most informative genes as indicated by the DNA microarray analysis. This approach can identify the origin of the CUP sample while avoiding the expense of performing unnecessary IHC testing. The IHC can be used to confirm the microarray findings.

The biomarker patterns or biomarker signature sets of the cancer or tumor can be used to determine a therapeutic agent or therapeutic protocol that is capable of interacting with the biomarker pattern or signature set. For example, with advanced breast cancer, immunohistochemistry analysis can be used to determine one or more gene expressed proteins that are overexpressed. Accordingly, a biomarker pattern or biomarker signature set can be identified for advanced stage breast cancer and a therapeutic agent or therapeutic protocol can be identified which is capable of interacting with the biomarker pattern or signature set.

These examples of biomarker patterns or biomarker signature sets for advanced stage breast cancer are just one example of the extensive number of biomarker patterns or biomarker signature sets for a number of advanced stage diseases or cancers that can be identified from the tables depicted in FIGS. 26-31. In addition, a number of non disease specific therapies or therapeutic protocols may be identified for treating patients with these biomarker patterns or biomarker signature sets by using method steps of the present invention described above such as depicted in FIGS. 1-2 and FIGS. 5-14.

The biomarker patterns and/or biomarker signature sets disclosed in the table depicted in FIGS. 26 and 28, and the tables depicted in FIGS. 27 and 30 may be used for a number of purposes including, but not limited to, specific cancer/disease detection, specific cancer/disease treatment, and identification of new drug therapies or protocols for specific cancers/diseases. The biomarker patterns and/or biomarker signature sets disclosed in the table depicted in FIGS. 26 and 28, and the tables depicted in FIGS. 27 and 30 can also represent drug resistant expression profiles for the specific tumor type or cancer type. The biomarker patterns and/or biomarker signature sets disclosed in the table depicted in FIGS. 26 and 28, and the tables depicted in FIGS. 27 and 30 represent advanced stage drug resistant profiles.

The biomarker patterns and/or biomarker signature sets can comprise at least one biomarker. In yet other embodiments, the biomarker patterns or signature sets can comprise at least 2, 3, 4, 5, 6, 7, 8, 9, or 10 biomarkers. In some embodiments, the biomarker signature sets or biomarker patterns can comprise at least 15, 20, 30, 40, 50, or 60 biomarkers. In some embodiments, the biomarker signature sets or biomarker patterns can comprise at least 70, 80, 90, 100, 200, 300, 400, 500, 600, 700, 800, 900, 1000, 2000, 3000, 4000, 5000, 6000, 7000, 8000, 9000, 10,000, 15,000, 20,000, 25,000, 30,000, 35,000, 40,000, 45,000 or 50,000 biomarkers. Analysis of the one or more biomarkers can be by one or more methods. For example, analysis of 2 biomarkers can be performed using microarrays. Alternatively, one biomarker may be analyzed by IHC and another by microarray. Any such combinations of methods and biomarkers are contemplated herein.

The one or more biomarkers can be selected from the group consisting of, but not limited to: Her2/Neu, ER, PR, c-kit, EGFR, MLH1, MSH2, CD20, p53, Cyclin D1, bc12, COX-2, Androgen receptor, CD52, PDGFR, AR, CD25, VEGF, HSP90, PTEN, RRM1, SPARC, Survivin, TOP2A, BCL2, HIF1A, AR, ESR1, PDGFRA, KIT, PDGFRB, CDW52, ZAP70, PGR, SPARC, GART, GSTP1, NFKBIA, MSH2, TXNRD1, HDAC1, PDGFC, PTEN, CD33, TYMS, RXRB, ADA, TNF, ERCC3, RAF1, VEGF, TOP1, TOP2A, BRCA2, TK1, FOLR2, TOP2B, MLH1, IL2RA, DNMT1, HSPCA, ERBR2, ERBB2, SSTR1, VHL, VDR, PTGS2, POLA, CES2, EGFR, OGFR, ASNS, NFKB2, RARA, MS4A1, DCK, DNMT3A, EREG, Epiregulin, FOLR1, GNRH1, GNRHR1, FSHB, FSHR, FSHPRH1, folate receptor, HGF, HIG1, IL13RA1, LTB, ODC1, PPARG, PPARGC1, Lymphotoxin Beta Receptor, Myc, Topoisomerase II, TOPO2B, TXN, VEGFC, ACE2, ADH1C, ADH4, AGT, AREG, CA2, CDK2, caveolin, NFKB1, ASNS, BDCA1, CD52, DHFR, DNMT3B, EPHA2, FLT1, HSP90AA1, KDR, LCK, MGMT, RRM1, RRM2, RRM2B, RXRG, SRC, SSTR2, SSTR3, SSTR4, SSTR5, VEGFA, or YES1.

For example, a biological sample from an individual can be analyzed to determine a biomarker pattern or biomarker signature set that comprises a biomarker such as HSP90, Survivin, RRM1, SSTRS3, DNMT3B, VEGFA, SSTR4, RRM2, SRC, RRM2B, HSP90AA1, STR2, FLT1, SSTR5, YES1, BRCA1, RRM1, DHFR, KDR, EPHA2, RXRG, or LCK. In other embodiments, the biomarker SPARC, HSP90, TOP2A, PTEN, Survivin, or RRM1 forms part of the biomarker pattern or biomarker signature set. In yet other embodiments, the biomarker MGMT, SSTRS3, DNMT3B, VEGFA, SSTR4, RRM2, SRC, RRM2B, HSP90AA1, STR2, FLT1, SSTR5, YES1, BRCA1, RRM1, DHFR, KDR, EPHA2, RXRG, CD52, or LCK is included in a biomarker pattern or biomarker signature set. In still other embodiments, the biomarker hENT1, cMet, P21, PARP-1, TLE3 or IGF1R is included in a biomarker pattern or biomarker signature set.

The expression level of HSP90, Survivin, RRM1, SSTRS3, DNMT3B, VEGFA, SSTR4, RRM2, SRC, RRM2B, HSP90AA1, STR2, FLT1, SSTR5, YES1, BRCA1, RRM1, DHFR, KDR, EPHA2, RXRG, or LCK can be determined and used to identify a therapeutic for an individual. The expression level of the biomarker can be used to form a biomarker pattern or biomarker signature set. Determining the expression level can be by analyzing the levels of mRNA or protein, such as by microarray analysis or IHC. In some embodiments, the expression level of a biomarker is performed by IHC, such as for SPARC, TOP2A, or PTEN, and used to identify a therapeutic for an individual. The results of the IHC can be used to form a biomarker pattern or biomarker signature set. In yet other embodiments, a biological sample from an individual or subject is analyzed for the expression level of CD52, such as by determining the mRNA expression level by methods including, but not limited to, microarray analysis. The expression level of CD52 can be used to identify a therapeutic for the individual. The expression level of CD52 can be used to form a biomarker pattern or biomarker signature set. In still other embodiments, the biomarkers hENT1, cMet, P21, PARP-1, TLE3 and/or IGF1R are assessed to identify a therapeutic for the individual.

As described herein, the molecular profiling of one or more targets can be used to determine or identify a therapeutic for an individual. For example, the expression level of one or more biomarkers can be used to determine or identify a therapeutic for an individual. The one or more biomarkers, such as those disclosed herein, can be used to form a biomarker pattern or biomarker signature set, which is used to identify a therapeutic for an individual. In some embodiments, the therapeutic identified is one that the individual has not previously been treated with. For example, a reference biomarker pattern has been established for a particular therapeutic, such that individuals with the reference biomarker pattern will be responsive to that therapeutic. An individual with a biomarker pattern that differs from the reference, for example the expression of a gene in the biomarker pattern is changed or different from that of the reference, would not be administered that therapeutic. In another example, an individual exhibiting a biomarker pattern that is the same or substantially the same as the reference is advised to be treated with that therapeutic. In some embodiments, the individual has not previously been treated with that therapeutic and thus a new therapeutic has been identified for the individual.

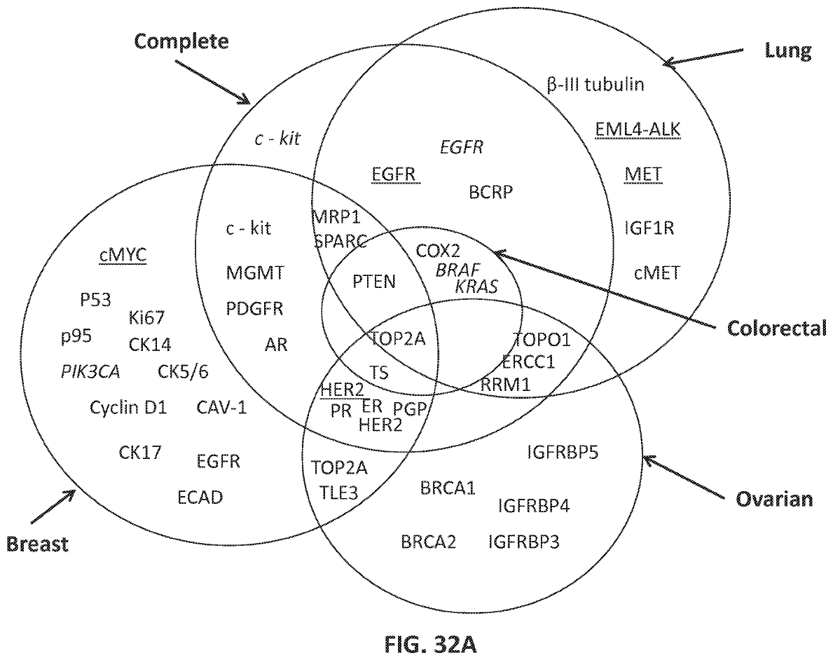

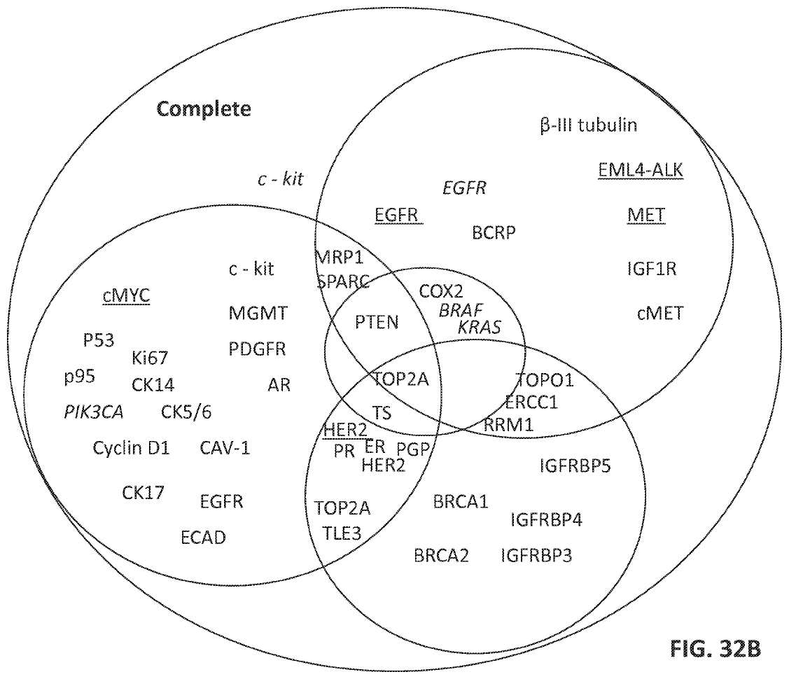

Molecular profiling according to the invention can take on a biomarker-centric or a therapeutic-centric point of view. Although the approaches are not mutually exclusive, the biomarker-centric approach focuses on sets of biomarkers that are expected to be informative for a tumor of a given tumor lineage, whereas the therapeutic-centric point approach identifies candidate therapeutics using biomarker panels that are lineage independent. In a biomarker-centric view, panels of specific biomarkers are run on different tumor types. See FIG. 32A. This approach provides a method of identifying a candidate therapeutic by collecting a sample from a subject with a cancer of known origin, and performing molecular profiling on the cancer for specific biomarkers depending on the origin of the cancer. The molecular profiling can be performed using any of the various techniques disclosed herein. As an example, FIG. 32A shows biomarker panels for breast cancer, ovarian cancer, colorectal cancer, lung cancer, and a “complete” profile to run on any cancer. In the figure, markers shown in italics are assessed using mutational analysis (e.g., sequencing approaches), marker shown underlined are analyzed by FISH, and the remainder are analyzed using IHC. DNA microarray profiling can be performed on any sample. The candidate therapeutic is selected based on the molecular profiling results according to the subject methods. An advantage to the bio-marker centric approach is only performing assays that are most likely to yield informative results. Another advantage is that this approach can focus on identifying therapeutics conventionally used to treat cancers of the specific lineage. In a therapeutic-centric approach, the biomarkers assessed are not dependent on the origin of the tumor. See FIG. 32B. This approach provides a method of identifying a candidate therapeutic by collecting a sample from a subject with a cancer, and performing molecular profiling on the cancer for a panel of biomarkers without regards to the origin of the cancer. The molecular profiling can be performed using any of the various techniques disclosed herein. As an example, in FIG. 32B, markers shown in italics are assessed using mutational analysis (e.g., sequencing approaches), marker shown underlined are analyzed by FISH, and the remainder are analyzed using IHC. DNA microarray profiling can be performed on any sample. The candidate therapeutic is selected based on the molecular profiling results according to the subject methods. An advantage to the therapeutic-marker centric approach is that the most promising therapeutics are identified only taking into account the molecular characteristics of the tumor itself. Another advantage is that the method can be preferred for a cancer of unidentified primary origin (CUP). In some embodiments, a hybrid of biomarker-centric and therapeutic-centric points of view is used to identify a candidate therapeutic. This method comprises identifying a candidate therapeutic by collecting a sample from a subject with a cancer of known origin, and performing molecular profiling on the cancer for a comprehensive panel of biomarkers, wherein a portion of the markers assessed depend on the origin of the cancer. For example, consider a breast cancer. A comprehensive biomarker panel is run on the breast cancer, e.g., the complete panel as shown in FIG. 32B, but additional sequencing analysis is performed on one or more additional markers, e.g., BRCA1 or any other marker with mutations informative for theranosis or prognosis of the breast cancer. Theranosis can be used to refer to the likely efficacy of a therapeutic treatment. Prognosis refers to the likely outcome of an illness. One of skill will apprecitate that the hybrid approach can be used to identify a candidate therapeutic for any cancer having additional biomarkers that provide theranostic or prognostic information, including the cancers disclosed herein.

Methods for providing a theranosis of disease include selecting candidate therapeutics for various cancers by assessing a sample from a subject in need thereof (i.e., suffering from a particular cancer). The sample is assessed by performing an immunohistochemistry (IHC) to determine of the presence or level of: AR, BCRP, c-KIT, ER, ERCC1, HER2, IGF1R, MET (also referred to herein as cMet), MGMT, MRP1, PDGFR, PGP, PR, PTEN, RRM1, SPARC, TOPO1, TOP2A, TS, COX-2, CK5/6, CK14, CK17, Ki67, p53, CAV-1, CYCLIN D1, EGFR, E-cadherin, p95, TLE3 or a combination thereof; performing a microarray analysis on the sample to determine a microarray expression profile on one or more (such as at least five, 10, 15, 20, 25, 30, 40, 50, 60, 70 or all) of: ABCC1, ABCG2, ADA, AR, ASNS, BCL2, BIRC5, BRCA1, BRCA2, CD33, CD52, CDA, CES2, DCK, DHFR, DNMT1, DNMT3A, DNMT3B, ECGF1, EGFR, EPHA2, ERBB2, ERCC1, ERCC3, ESR1, FLT1, FOLR2, FYN, CART, GNRH1, GSTP1, HCK, HDAC1, HIF1A, HSP90AA1, IGFBP3, IGFBP4, IGFBP5, IL2RA, KDR, KIT, LCK, LYN, MET, MGMT, MLH1, MS4A1, MSH2, NFKB1, NFKB2, NFKBIA, OGFR, PARP1, PDGFC, PDGFRA, PDGFRB, PGP, PGR, POLA1, PTEN, PTGS2, PTPN12, RAF1, RARA, RRM1, RRM2, RRM2B, RXRB, RXRG, SIK2, SPARC, SRC, SSTR1, SSTR2, SSTR3, SSTR4, SSTR5, TK1, TNF, TOP1, TOP2A, TOP2B, TXNRD1, TYMS, VDR, VEGFA, VHL, YES1, and ZAP70; comparing the results obtained from the IHC and microarray analysis against a rules database, wherein the rules database comprises a mapping of candidate treatments whose biological activity is known against a cancer cell that expresses one or more proteins included in the IHC expression profile and/or expresses one or more genes included in the microarray expression profile; and determining a candidate treatment if the comparison indicates that the candidate treatment has biological activity against the cancer.

Assessment can further comprise determining a fluorescent in-situ hybridization (FISH) profile of EGFR, HER2, cMYC, TOP2A, MET, or a combination thereof, comparing the FISH profile against a rules database comprising a mapping of candidate treatments predetermined as effective against a cancer cell having a mutation profile for EGFR, HER2, cMYC, TOP2A, MET, or a combination thereof, and determining a candidate treatment if the comparison of the FISH profile against the rules database indicates that the candidate treatment has biological activity against the cancer.

As explained further herein, the FISH analysis can be performed based on the origin of the sample. This can avoid unnecessary laboratory procedures and concomitant expenses by targeting analysis of genes that are known to play a role in a particular disorder, e.g., a particular type of cancer. In an embodiment, EGFR, HER2, cMYC, and TOP2A are assessed for breast cancer. In another embodiment, EGFR and MET are assessed for lung cancer. Alternately, FISH analysis of all of EGFR, HER2, cMYC, TOP2A, MET can be performed on a sample. The complete panel may be assessed, e.g., when a sample is of unknown or mixed origin, to provide a comprehensive view of an unusual sample, or when economies of scale dictate that it is more efficient to perform FISH on the entire panel than to make individual assessments.

In an additional embodiment, the sample is assessed by performing nucleic acid sequencing on the sample to determine a presence of a mutation of KRAS, BRAF, NRAS, PIK3CA (also referred to as PI3K), c-Kit, EGFR, or a combination thereof, comparing the results obtained from the sequencing against a rules database comprising a mapping of candidate treatments predetermined as effective against a cancer cell having a mutation profile for KRAS, BRAF, NRAS, PIK3CA, c-Kit, EGFR, or a combination thereof; and determining a candidate treatment if the comparison of the sequencing to the mutation profile indicates that the candidate treatment has biological activity against the cancer.

As explained further herein, the nucleic acid sequencing can be performed based on the origin of the sample. This can avoid unnecessary laboratory procedures and concomitant expenses by targeting analysis of genes that are known to play a role in a particular disorder, e.g., a particular type of cancer. In an embodiment, the sequences of PIK3CA and c-KIT are assessed for breast cancer. In another embodiment, the sequences of KRAS and BRAF are assessed for GI cancers such as colorectal cancer. In still another embodiment, the sequences of KRAS, BRAF and EGFR are assessed for lung cancer. Alternately, sequencing of all of KRAS, BRAF, NRAS, PIK3CA, c-Kit, EGFR can be performed on a sample. The complete panel may be sequenced, e.g., when a sample is of unknown or mixed origin, to provide a comprehensive view of an unusual sample, or when economies of scale dictate that it is more efficient to sequence the entire panel than to make individual assessments.

The genes and gene products used for molecular profiling, e.g., by microarray, IHC, FISH, sequencing, and/or PCR (e.g., qPCR), can be selected from those listed in Table 2, Table 6 or Table 25. In an embodiment, IHC is performed for one or more, e.g., 2, 3, 4, 5, 6, 7, 8, 9, 10, 15, 20 or more, of: AR, BCRP, CAV-1, CD20, CD52, CK 5/6, CK14, CK17, c-kit, CMET, COX-2, Cyclin D1, E-Cad, EGFR, ER, ERCC1, HER-2, IGF1R, Ki67, MGMT, MRP1, P53, p95, PDGFR, PGP, PR, PTEN, RRM1, SPARC, TLE3, TOPO1, TOPO2A, TS, TUBB3; expression analysis (e.g., microarray or RT-PCR) is performed on one or more, e.g. 2, 3, 4, 5, 6, 7, 8, 9, 10, 15, 20, 25, 30, 40, 50 or more, of: ABCC1, ABCG2, ADA, AR, ASNS, BCL2, BIRC5, BRCA1, BRCA2, CD33, CD52, CDA, CES2, cKit, c-MYC, DCK, DHFR, DNMT1, DNMT3A, DNMT3B, ECGF1, EGFR, EPHA2, ERCC1, ERCC3, ESR1, FLT1, FOLR2, FYN, GART, GNRH1, GSTP1, HCK, HDAC1, HER2/ERBB2, HIF1A, HSP90, IGFBP3, IGFBP4, IGFBP5, IL2RA, KDR, LCK, LYN, MET, MGMT, MLH1, MS4A1, MSH2, NFKB1, NFKB2, NFKBIA, OGFR, PARP1, PDGFC, PDGFRa, PDGFRA, PDGFRB, PGP, PGR, POLA1, PTEN, PTGS2, RAF1, RARA, ROS1, RRM1, RRM2, RRM2B, RXRB, RXRG, SIK2, SRC, SSTR1, SSTR2, SSTR3, SSTR4, SSTR5, SPARC, TK1, TNF, TOP2B, TOP2A, TOPO1, TXNRD1, TYMS, VDR, VEGFA, VHL, YES1, and ZAP70; fluorescent in-situ hybridization (FISH) is performed on 1, 2, 3, 4, 5, 6 or 7 of ALK, cMET, c-MYC, EGFR, HER-2, PIK3CA, and TOPO2A; and DNA sequencing or PCR are performed on 1, 2, 3, 4, 5 or 6 of BRAF, c-kit, EGFR, KRAS, NRAS, and PIK3CA. In an embodiment, all of these genes and/or the gene products thereof are assessed.

Assessing one or more biomarkers disclosed herein can be used for characterizing any of the cancers disclosed herein. Characterizing includes the diagnosis of a disease or condition, the prognosis of a disease or condition, the determination of a disease stage or a condition stage, a drug efficacy, a physiological condition, organ distress or organ rejection, disease or condition progression, therapy-related association to a disease or condition, or a specific physiological or biological state.

A cancer in a subject can be characterized by obtaining a biological sample from a subject and analyzing one or more biomarkers from the sample. For example, characterizing a cancer for a subject or individual may include detecting a disease or condition (including pre-symptomatic early stage detecting), determining the prognosis, diagnosis, or theranosis of a disease or condition, or determining the stage or progression of a disease or condition. Characterizing a cancer can also include identifying appropriate treatments or treatment efficacy for specific diseases, conditions, disease stages and condition stages, predictions and likelihood analysis of disease progression, particularly disease recurrence, metastatic spread or disease relapse. Characterizing can also be identifying a distinct type or subtype of a cancer. The products and processes described herein allow assessment of a subject on an individual basis, which can provide benefits of more efficient and economical decisions in treatment.

In an aspect, characterizing a cancer includes predicting whether a subject is likely to respond to a treatment for the cancer. As used herein, a “responder” responds to or is predicted to respond to a treatment and a “non-responder” does not respond or is predicted to not respond to the treatment. Biomarkers can be analyzed in the subject and compared to biomarker profiles of previous subjects that were known to respond or not to a treatment. If the biomarker profile in a subject more closely aligns with that of previous subjects that were known to respond to the treatment, the subject can be characterized, or predicted, as a responder to the treatment. Similarly, if the biomarker profile in the subject more closely aligns with that of previous subjects that did not respond to the treatment, the subject can be characterized, or predicted as a non-responder to the treatment.

The sample used for characterizing a cancer can be any disclosed herein, including without limitation a tissue sample, tumor sample, or a bodily fluid. Bodily fluids that can be used included without limitation peripheral blood, sera, plasma, ascites, urine, cerebrospinal fluid (CSF), sputum, saliva, bone marrow, synovial fluid, aqueous humor, amniotic fluid, cerumen, breast milk, broncheoalveolar lavage fluid, semen (including prostatic fluid), Cowper's fluid or pre-ejaculatory fluid, female ejaculate, sweat, fecal matter, hair, tears, cyst fluid, pleural and peritoneal fluid, pericardial fluid, malignant effusion, lymph, chyme, chyle, bile, interstitial fluid, menses, pus, sebum, vomit, vaginal secretions, mucosal secretion, stool water, pancreatic juice, lavage fluids from sinus cavities, bronchopulmonary aspirates or other lavage fluids. In an embodiment, the sample comprises vesicles. The biomarkers can be associated with the vesicles. In some embodiments, vesicles are isolated from the sample and the biomarkers associated with the vesicles are assessed.

### Comprehensive and Standard-of-Care Molecular Profiling

Molecular profiling according to the invention can be used to guide treatment selection for cancers at any stage of disease or prior treatment. Molecular profiling comprises assessment of DNA mutations, gene rearrangements, gene copy number variation, RNA expression, protein expression, as well as assessment of other biological entities and phenomena that can inform clinical decision making. In some embodiments, the methods herein are used to guide selection of candidate treatments using the standard of care treatments for a particular type or lineage of cancer. Profiling of biomarkers that implicate standard-of-care treatments may be used to assist in treatment selection for a newly diagnosed cancer having multiple treatment options. Such profiling may be referred to herein as “select” profiling. Standard-of-care treatments may comprise NCCN on-compendium treatments or other standard treatments used for a cancer of a given lineage. One of skill will appreciate that such profiles can be updated as the standard of care and/or availability of experimental agents for a given disease lineage change. In other embodiments, molecular profiling is performed for additional biomarkers to identify treatments as beneficial or not beyond that go beyond the standard-of-care for a particular lineage or stage of the cancer. Such “comprehensive” profiling can be performed to assess a wide panel of druggable or drug-associated biomarker targets for any biological sample or specimen of interest. One of skill will appreciate that the select profiles generally comprise subsets of the comprehensive profile. The comprehensive profile can also be used to guide selection of candidate treatments for any cancer at any point of care. The comprehensive profile may also be preferable when standard-of-care treatments not expected to provide further benefit, such as in the salvage treatment setting for recurrent cancer or wherein all standard treatments have been exhausted. For example, the comprehensive profile may be used to assist in treatment selection when standard therapies are not an option for any reason including, without limitation, when standard treatments have been exhausted for the patient. The comprehensive profile may be used to assist in treatment selection for highly aggressive or rare tumors with uncertain treatment regimens. For example, a comprehensive profile can be used to identify a candidate treatment for a newly diagnosed case or when the patient has exhausted standard of care therapies or has an aggressive disease. In practice, molecular profiling according to the invention has indeed identified beneficial therapies for a cancer patient when all standard-of-care treatments were exhausted the treating physician was unsure of what treatment to select next. See the Examples herein. One of skill in the art will appreciate that by its very nature a comprehensive molecular profiling can be used to select a therapy for any appropriate indication independent of the nature of the indication (e.g., source, stage, prior treatment, etc). However, in some embodiments, a comprehensive molecular profile is tailored for a particular indication. For example, biomarkers associated with treatments that are known to be ineffective for a cancer from a particular lineage or anatomical origin may not be assessed as part of a comprehensive molecular profile for that particular cancer. Similarly, biomarkers associated with treatments that have been previously used and failed for a particular patient may not be assessed as part of a comprehensive molecular profile for that particular patient. In yet another non-limiting example, biomarkers associated with treatments that are only known to be effective for a cancer from a particular anatomical origin may only be assessed as part of a comprehensive molecular profile for that particular cancer. One of skill will further appreciate that the comprehensive molecular profile can be updated to reflect advancements, e.g., new treatments, new biomarker-drug associations, and the like, as available.

### Molecular Intelligence Profiles (5.0)

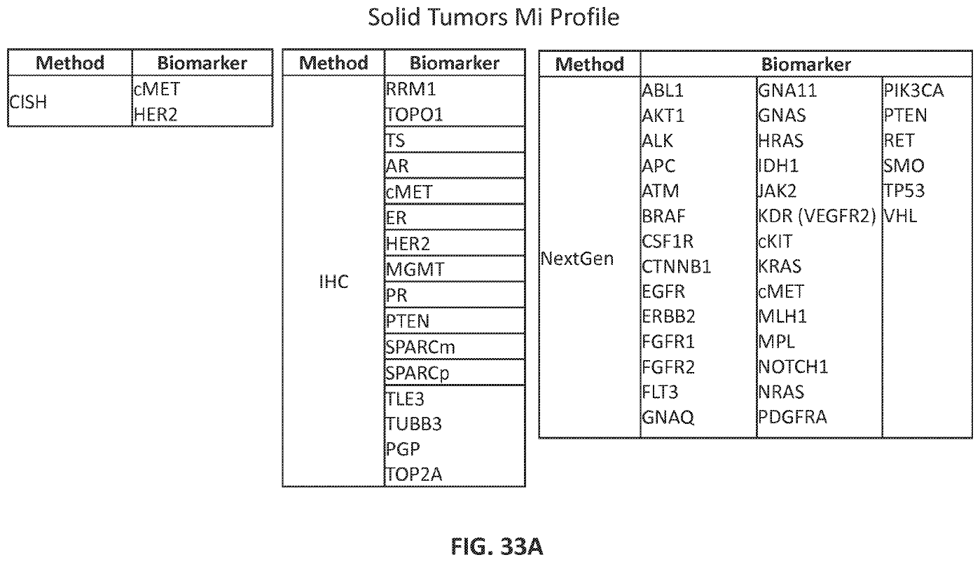

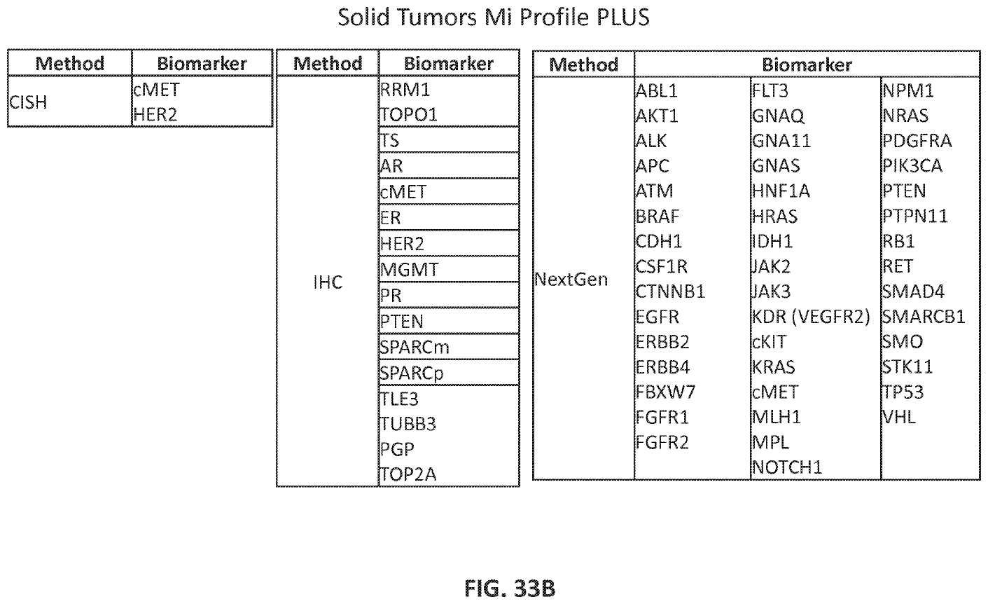

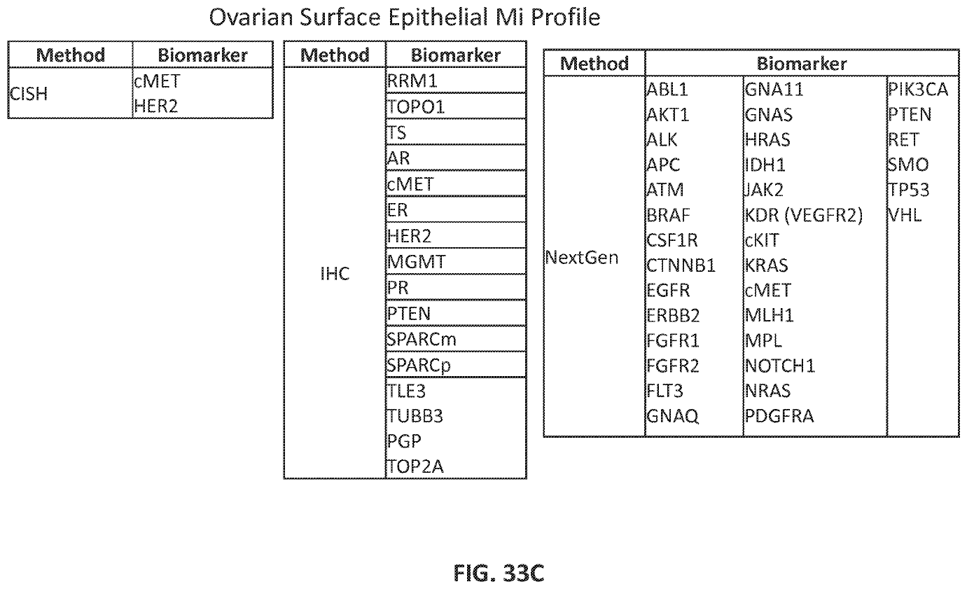

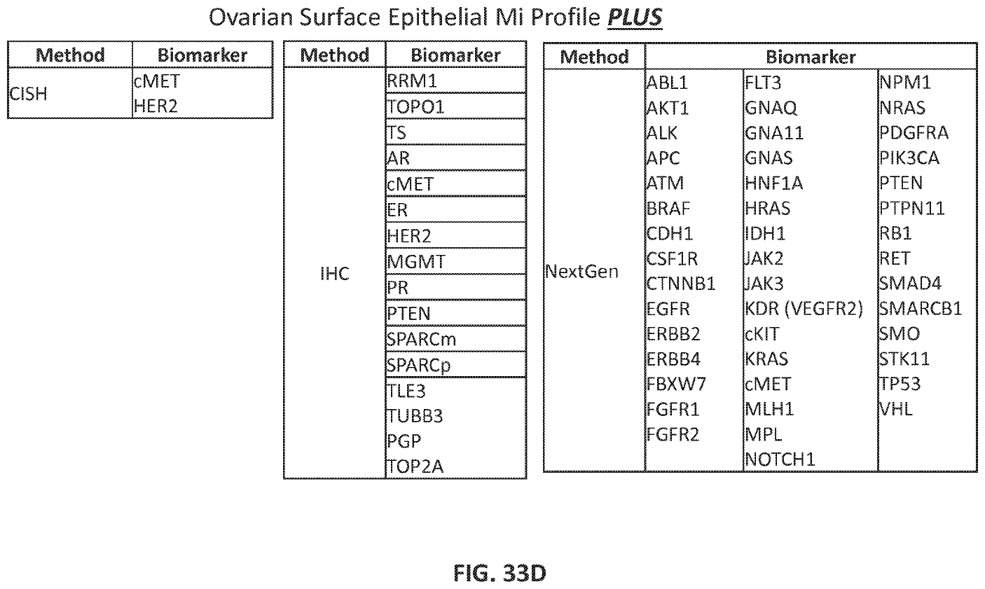

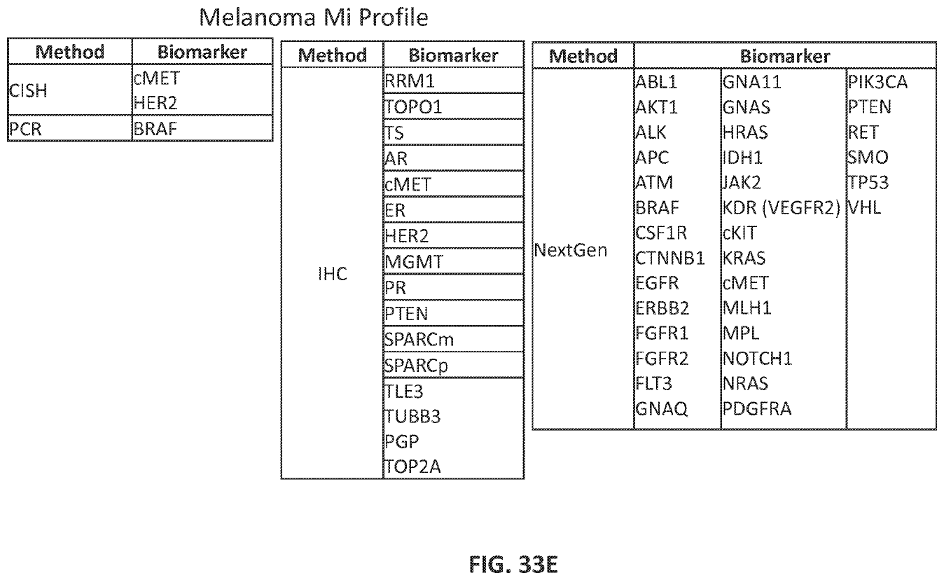

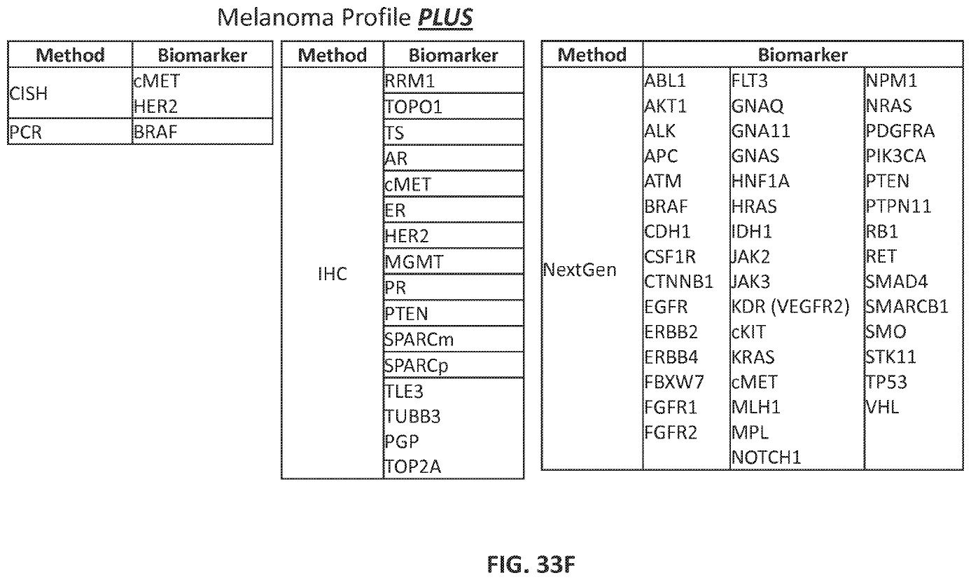

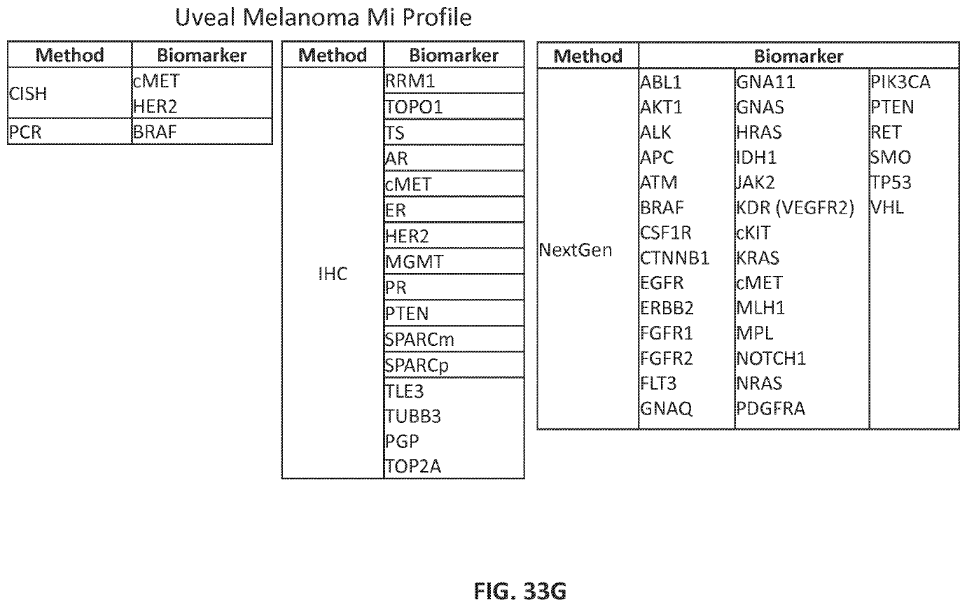

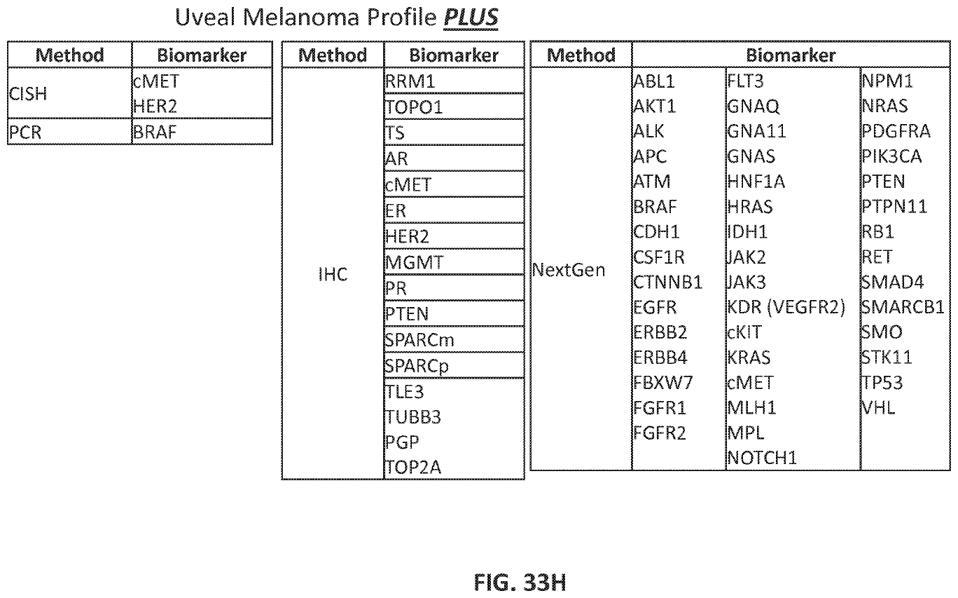

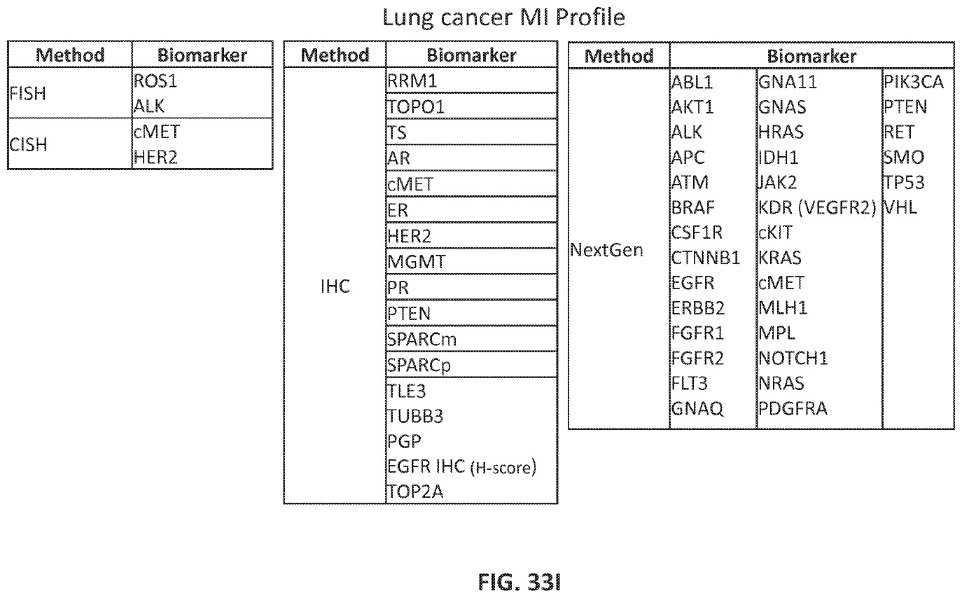

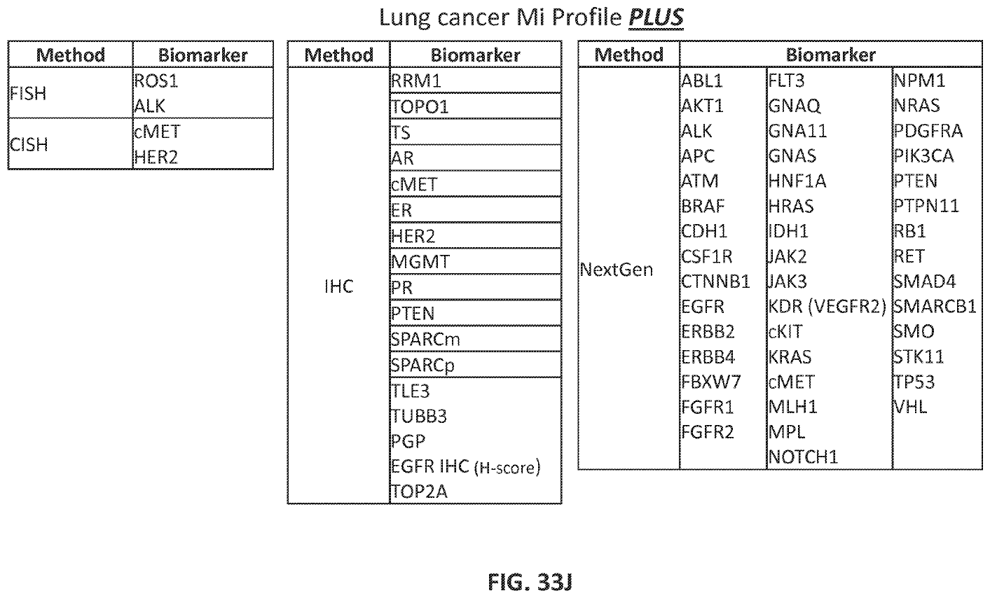

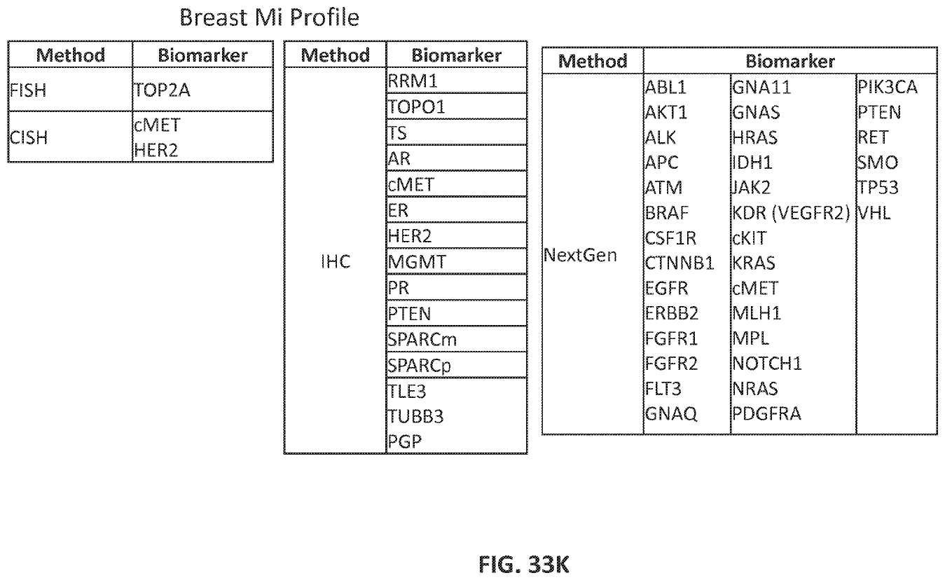

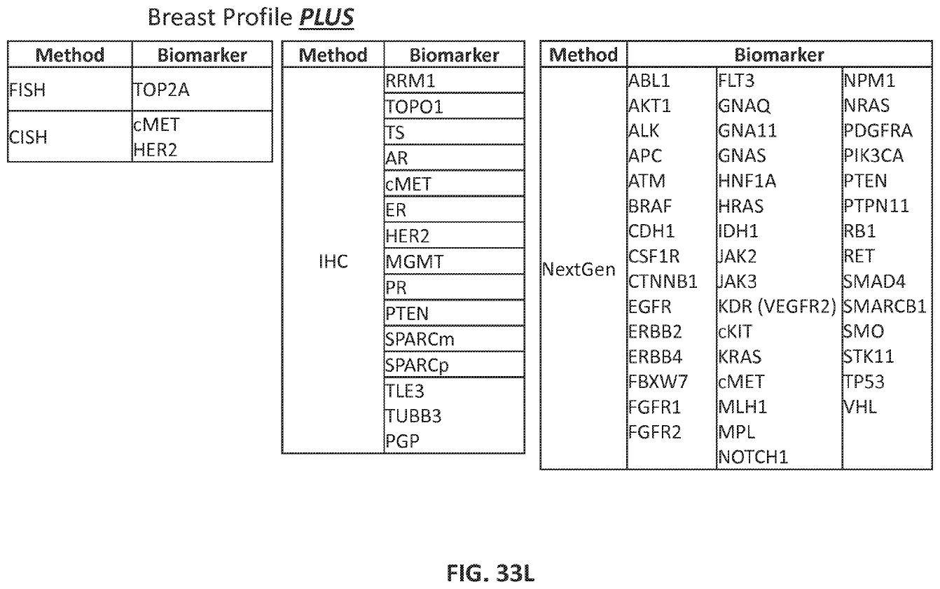

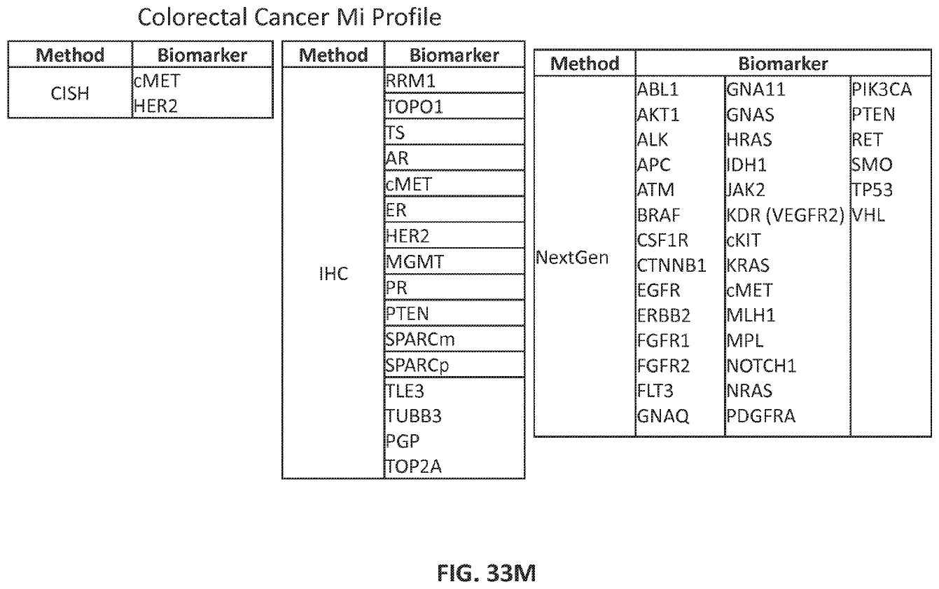

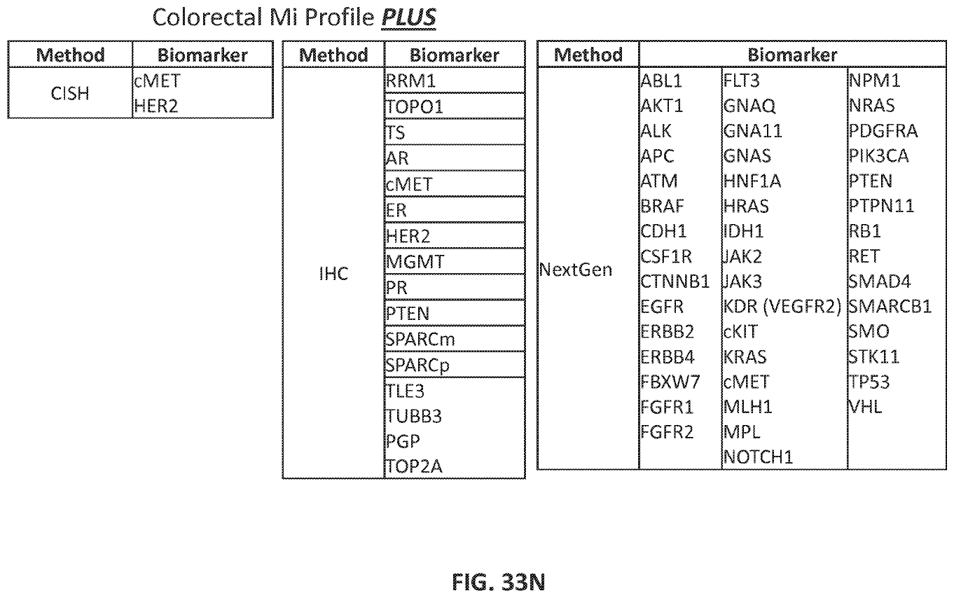

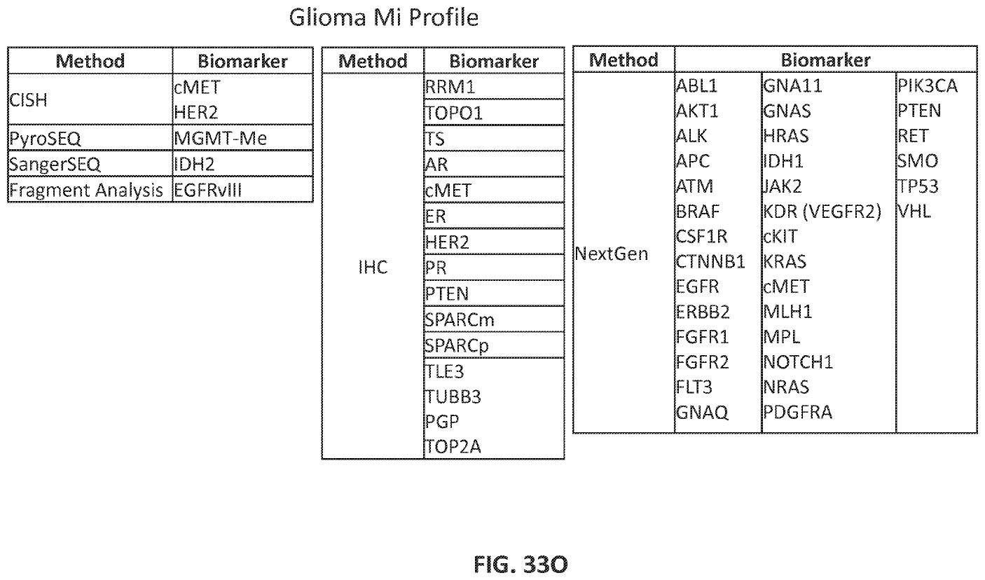

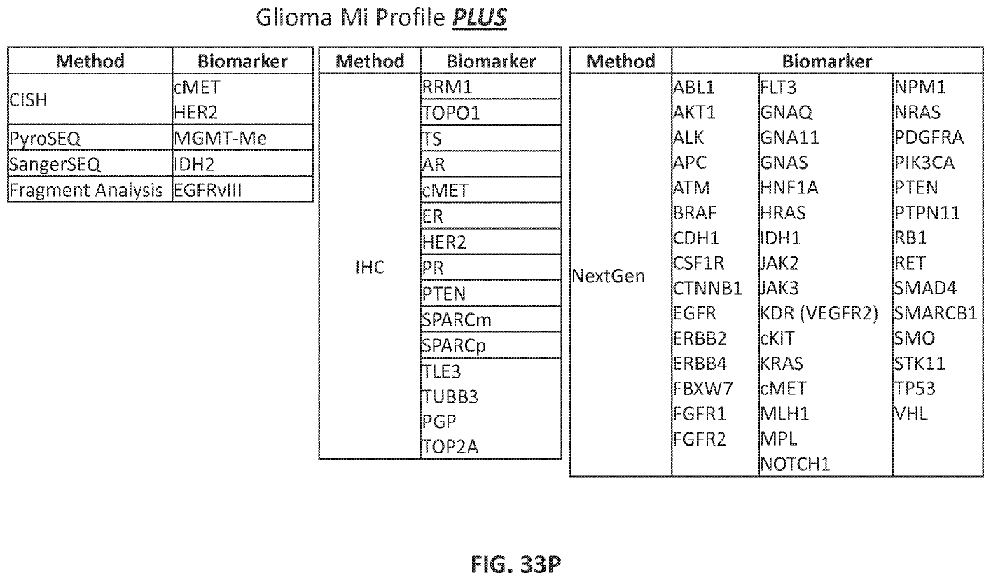

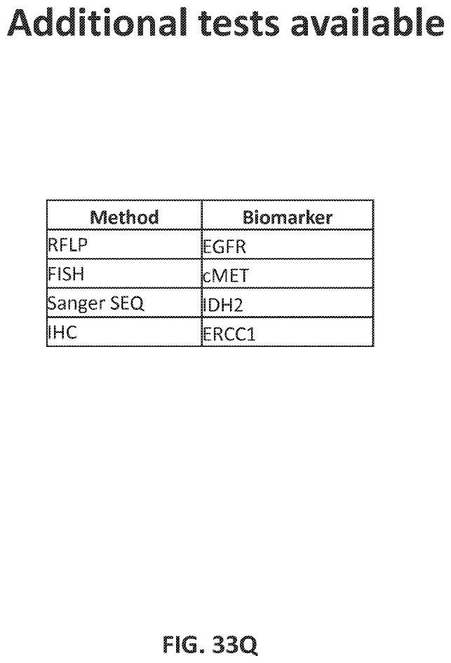

The invention provides molecular intelligence (MI) molecular profiles using a variety of techniques to assess panels of biomarkers in order to select or not select a candidate therapeutic for treating a cancer. Such techniques comprise IHC for expression profiling, CISH/FISH for DNA copy number, and Sanger, Pyrosequencing, PCR, RFLP, fragment analysis and Next Generation sequencing for mutational analysis. Such profiles are described in FIGS. 33A-33Q. The profiling is performed using the rules for the biomarker-drug associations for the various cancer lineages as described for FIGS. 33A-33Q and Tables 7-24. MI profiles for all solid tumors or that have additional analyses based on tumor lineage include NextGen analysis of a panel of biomarkers linked to known therapies and clinical trials. The MI profiles can further be expanded to “MI PLUS” profiles that include sequencing of set of genes that are known to be involved in cancer and have alternative clinical utilities including predictive, prognostic or diagnostic uses.

The biomarkers which comprise the molecular intelligence molecular profiles can include genes or gene products that are known to be associated directly with a particular drug or class of drugs. The biomarkers can also be genes or gene products that interact with such drug associated targets, e.g., as members of a common pathway. The biomarkers can be selected from Table 2. In some embodiments, the genes and/or gene products included in the molecular intelligence (MI) molecular profiles are selected from Table 6.

TABLE 6
Exemplary Genes and Gene Products and Related Therapies

| Biomarker | Description |
|---|---|
| ALK | ALK rearrangements may indicate the fusion of ALK (anaplastic lymphoma |
| | kinase) gene with fusion partners, such as EML4. EML4-ALK fusion results in the |
| | pathologic expression of a fusion protein with constitutively active ALK kinase, |
| | resulting in aberrant activation of downstream signaling pathways including RAS- |
| | ERK, JAK3-STAT3 and PI3K-AKT. Patients with ALK rearrangements such as |
| | EML4-ALK are likely to respond to the ALK-targeted agent crizotinib. |
| AR | The androgen receptor (AR) is a member of the nuclear hormone receptor |
| | superfamily. Prostate tumor dependency on androgens/AR signaling is the basis |
| | for hormone withdrawal, or androgen ablation therapy, to treat men with prostate |
| | cancer. Androgen receptor antagonists as well as agents which block androgen |
| | production are indicated for the treatment of AR expressing prostate cancers. |
| AREG | AREG, also known as amphiregulin, is a ligand of the epidermal growth factor |
| | receptor. Overexpression of AREG in primary colorectal cancer patients has been |
| | associated with increased clinical benefit from cetuximab in KRAS wildtype |
| | patients. |
| BRAF | BRAF encodes a protein belonging to the raf/mil family of serine/threonine protein |
| | kinases. This protein plays a role in regulating the MAP kinase/ERK signaling |
| | pathway initiated by EGFR activation, which affects cell division, differentiation, |
| | and secretion. Patients with mutated BRAF genes have a reduced likelihood of |
| | response to EGFR targeted monoclonal antibodies, such as cetuximab in colorectal |
| | cancer. A BRAF enzyme inhibitor, vemurafenib, was approved by FDA to treat |
| | unresectable or metastatic melanoma patients harboring BRAF V600E mutations. |
| BRCA1 | BRCA1, breast cancer type 1 susceptibility gene, is a gene involved in cell growth, |
| | cell division, and DNA-damage repair. Low expression of the BRCA1 gene has |
| | been associated with clinical benefit from cisplatin and carboplatin in cancers of |
| | the lung and ovary. |
| c-kit | c-Kit is a cytokine receptor expressed on the surface of hematopoietic stem cells as |
| | well as other cell types. This receptor binds to stem cell factor (SCF, a cell growth |
| | factor). As c-Kit is a receptor tyrosine kinase, ligand binding causes receptor |
| | dimerization and initiates a phosphorylation cascade resulting in changes in gene |
| | expression. These changes affect cell proliferation, apoptosis, chemotaxis and |
| | adhesion. c-Kit is inhibited by multi-targeted agents including imatinib, sunitinib |
| | and sorafenib. |
| cMET | C-Met is a tyrosine kinase receptor for hepatocyte growth factor (HGF) or scatter |
| | factor (SF) and is overexpressed and amplified in a wide range of tumors. cMET |
| | overexpression has been associated with a more aggressive biology and a worse |
| | prognosis in many human malignancies. Amplification or overexpression of cMET |
| | has been implicated in the development of acquired resistance to erlotinib and |
| | gefitinib inNSCLC. |
| EGFR | EGFR (epidermal growth factor receptor) is a receptor tyrosine kinase and its |
| | abnormalities contribute to the growth and proliferation of many human cancers. |
| | Sensitizing mutations are commonly detected in NSCLC and patients harboring |
| | such mutations may respond to EGFR-targeted tyrosine kinase inhibitors including |
| | erlotinib and gefitinib. Lung cancer patients overexpressing EGFR protein are |
| | known to respond to the EGFR monoclonal antibody, cetuximab. Increased gene |
| | expression of EGFR is associated with response to irinotecan containing regimen |
| | in colorectal cancer patients. |
| ER | The estrogen receptor (ER) is a member of the nuclear hormone family of |
| | intracellular receptors which is activated by the hormone estrogen. It functions as a |
| | DNA binding transcription factor to regulate estrogen-mediated gene expression. |
| | Estrogen receptors overexpressing breast cancers are referred to as “ER positive.” |
| | Estrogen binding to ER on cancer cells leads to cancer cell proliferation. Breast |
| | tumors over-expressing ER are indicated for treatment with hormone-based anti- |
| | estrogen therapy. |
| ERBB3 | ERBB3 encodes for HER3, a member of the epidermal growth factor receptor |
| | (EGFR) family. This protein forms heterodimers with other EGF receptor family |
| | members which do have kinase activity. Amplification and/or overexpression of |
| | ERBB3 have been reported in numerous cancers, including breast cancer. ERBB3 |
| | is a target for drug development. |
| ERCC1 | Nucleotide excision repair (NER) is a DNA repair mechanism necessary for the |
| | repair of DNA damage from a vast variety of sources including chemicals and |
| | ultraviolet (UV) light from the sun. ERCC1 (excision repair cross- |
| | complementation group 1) is an important enzyme in the NER pathway. Platinum- |
| | based drugs induce DNA cross-links that interfere with DNA replication. Tumors |
| | with low ERCC1 expression and, hence, less DNA repair capacity, are more likely |
| | to benefit from platinum-based DNA damaging agents. |
| EREG | EREG, also known as epiregulin, is a ligand of the epidermal growth factor |
| | receptor. Overexpression of EREG in primary colorectal cancer patients has been |
| | shown to significantly predict clinical outcome in KRAS wildtype patients treated |
| | with cetuximab indicating ligand driven autocrine oncogenic EGFR signaling. |
| GNA11 | G proteins are a family of heterotrimeric proteins coupling seven-transmembrane |
| | domain receptor. These heterotrimeric proteins are composed of three subunits: |
| | Galpha, Gbeta, and Ggamma. The GNA11 gene encodes the alpha-11 subunit |
| | (Galphal 1). Recent data suggests that over half of uveal melanoma patients lacking |
| | a mutation in GNAQ exhibit mutations in GNA11. Clinical trials are underway |
| | with HDAC inhibitors and MEK inhibitors in patients harboring GNA11 |
| | mutations. |
| GNAQ | G proteins are a family of heterotrimeric proteins coupling seven-transmembrane |
| | domain receptors. G proteins are potential drivers of MAPK activation. In uveal |
| | melanomas 46-53% of patients exhibit a GNAQ mutation which encodes the q |
| | class of G-protein alpha subunit. Clinical trials are underway with HDAC |
| | inhibitors and MEK inhibitors in patients harboring GNAQ mutations. |
| Her2/Neu | ErbB2/Her2 encodes a member of the epidermal growth factor (EGF) receptor |
| | family of receptor tyrosine kinases. Her2 has no ligand-binding domain of its own |
| | and, therefore, cannot bind growth factors. It does, however, bind tightly to other |
| | ligand-bound EGF receptor family members to form a heterodimer and enhances |
| | kinase-mediated activation of downstream signaling pathways leading to cell |
| | proliferation. Her2 is overexpressed in 15-30% of newly diagnosed breast cancers |
| | and is also expressed in various other cancers. Her2 is a target for the monoclonal |
| | antibodies trastuzumab and pertuzumab which bind to the receptor extracellularly; |
| | the kinase inhibitor lapatinib binds and blocks the receptor intracellularly. |
| IDH2 | IDH2 encodes for the mitochondrial form of isocitrate dehydrogenase, a key |
| | enzyme in the citric acid cycle, which is essential for cell respiration. Mutation in |
| | IDH2 may results in impaired catalytic function of the enzyme, and cause the |
| | overproduction of an onco-metabolite, 2-hydroxy-glutarate, which can extensively |
| | alter the methylation profile in cancer. IDH2 mutation is mutually exclusive of |
| | IDH1 mutation, and has been found in 2% of gliomas and 10% of AML, as well as |
| | in cartilaginous tumors and cholangiocarcinoma. In gliomas, IDH2 mutations are |
| | associated with lower grade astrocytomas, oligodendrogliomas (grade II/III), as |
| | well as secondary glioblastoma (transformed from a lower grade glioma), and are |
| | associated with a better prognosis. In secondary glioblastoma, preliminary |
| | evidence suggests that IDH2 mutation may associate with a better response to |
| | alkylating agent temozolomide. IDH mutations have also been suggested to |
| | associate with a benefit from using hypomcthylating agents in cancers including |
| | AML. Various clinical trials investigating agents which target this gene and/or its |
| | downstream or upstream effectors may be available, which include the following: |
| | NCT01534845, NCT01537744. Germline IDH2 mutation has been indicated to |
| | associate with a rare inherited neurometabolic disorder D-2-hydroxyglutaric |
| | aciduria. |
| KRAS | Proto-oncogene of the Kirsten murine sarcoma virus (KRAS) is a signaling |
| | intermediate involved in many signaling cascades including the EGFR pathway. |
| | Mutations at activating hotspots are associated with resistance to EGFR tyrosine |
| | kinase inhibitors (erlotinib, gefitinib) and monoclonal antibodies (cetuximab, |
| | panitumumab). |
| MGMT | O-6-methylguanine-DNA methyltransferase (MGMT) encodes a DNA repair |
| | enzyme. Loss of MGMT expression leads to compromised DNA repair in cells and |
| | may play a significant role in cancer formation. Low MGMT expression has been |
| | correlated with response to alkylating agents like temozolomide and dacarbazine. |
| | MGMT expression can be downregulated by promoter hyper methylation. |
| NRAS | NRAS is an oncogene and a member of the (GTPase) ras family, which includes |
| | KRAS and HRAS. This biomarker has been detected in multiple cancers including |
| | melanoma, colorectal cancer, AML and bladder cancer. Evidence suggests that an |
| | acquired mutation in NRAS may be associated with resistance to vemurafenib in |
| | melanoma patients. In other cancers, e.g., colorectal cancer, NRAS mutation is |
| | associated with resistance to EGFR-targeted monoclonal antibodies. |
| PGP | P-glycoprotein (MDR1, ABCB1) is an ATP-dependent, transmembrane drug efflux |
| | pump with broad substrate specificity, which pumps antitumor drugs out of cells. |
| | Its expression is often induced by chemotherapy drugs and is thought to be a major |
| | mechanism of chemotherapy resistance. Overexpression of PGP is associated with |
| | resistance to anthracylines (doxorubicin, epirubicin). PGP remains the most |
| | important and dominant representative of Multi-Drug Resistance phenotype and is |
| | correlated with disease state and resistant phenotype. |
| PIK3CA | The hot spot missense mutations in the gene PIK3CA are present in various |
| | malignancies, e.g., breast, colon and NSCLC, resulting in activation of the PI3 |
| | kinase pathway. This pathway is an active target for drug development. PIK3CA |
| | mutations have been associated with benefit from mTOR inhibitors (everolimus, |
| | temsirolimus). Evidence suggests that breast cancer patients with activation of the |
| | PI3K pathway due to PTEN loss or PIK3CA mutation/amplification have a |
| | significantly shorter survival following trastuzumab treatment. PIK3CA mutated |
| | (exon 20) colorectal cancer patients are less likely to respond to EGFR targeted |
| | monoclonal antibody therapy. |
| PR | The progesterone receptor (PR or PGR) is an intracellular steroid receptor that |
| | specifically binds progesterone, an important hormone that fuels breast cancer |
| | growth. PR positivity in a tumor indicates that the tumor is more likely to be |
| | responsive to hormone therapy by anti-estrogens, aromatase inhibitors and |
| | progestogens. |
| PTEN | PTEN (phosphatase and tensin homolog) is a tumor suppressor gene that prevents |
| | cells from proliferating. Loss of PTEN protein is one of the most common |
| | occurrences in multiple advanced human cancers. PTEN is an important mediator |
| | in signaling downstream of EGFR, and its loss is associated with reduced benefit to |
| | trastuzumab and EGFR-targeted therapies. Intra-tumoral PTEN loss has been |
| | associated with benefit from mTOR inhibitors (everolimus, temsirolimus). |
| RET | The RET proto-oncogene is a member of the cadherin superfamily and encodes a |
| | receptor tyrosine kinase cell-surface molecule involved in numerous cellular |
| | mechanisms including cell proliferation, neuronal navigation, cell migration, and |
| | cell differentiation upon binding with glial cell derived neurotrophic factor family |
| | ligands.. Gain of function mutations in RET are associated with the development of |
| | various types of human cancers. Vandetanib is a tyrosine kinase inhibitor that can |
| | inhibit several receptors, including VEGFR, EGFR, and RET. |
| ROS1 | ROS1 (c-ros oncogene 1, receptor tyrosine kinase) is a tyrosine kinase that plays a |
| | role in epithelial cell differentiation and regionalization of the proximal epididymal |
| | epithelium. ROS1 may activate several downstream signaling pathways related to |
| | cell differentiation, proliferation, growth and survival including the PI3 kinase- |
| | mTOR signaling pathway. TKI inhibitors such as crizotinib or other ROS1 |
| | inhibitor compounds can have benefit when mutations or rearrangements in ROS1 |
| | are identified. |
| RRM1 | Ribonucleotide reductase subunit M1 (RRM1) is a component of the |
| | ribonucleotide reductase holoenzyme consisting of M1 and M2 subunits. The |
| | ribonucleotide reductase is a rate-limiting enzyme involved in the production of |
| | nucleotides required for DNA synthesis. Gemcitabine is a deoxycitidine analogue |
| | which inhibits ribonucleotide reductase activity. High RRM1 level is associated |
| | with resistance to gemcitabine. |
| SPARC | SPARC (secreted protein acidic and rich in cysteine) is a calcium-binding |
| | matricellular glycoprotein secreted by many types of cells. Studies indicate SPARC |
| | over-expression improves the response to the anticancer drug, nab-paclitaxel. The |
| | improved response is thought to be related to SPARC's role in accumulating |
| | albumin and albumin-targeted agents within tumor tissue. |
| TLE3 | TLE3 is a member of the transducin-like enhancer of split (TLE) family of proteins |
| | that have been implicated in tumorigenesis. It acts downstream of APC and beta- |
| | catenin to repress transcription of a number of oncogenes, which influence growth |
| | and microtubule stability. Studies indicate that TLE3 expression is associated with |
| | response to taxane therapy in various cancers, e.g., breast, ovarian and lung |
| | cancers. |
| TOP2A | TOPOIIA is an enzyme that alters the supercoiling of double-stranded DNA and |
| | allows chromosomal segregation into daughter cells. Due to its essential role in |
| | DNA synthesis and repair, and frequent overexpression in tumors, TOPOIIA is an |
| | ideal target for antineoplastic agents. In breast cancer, co-amplification of |
| | TOPOIIA and HER2 has been associated with benefit from anthracycline-based |
| | therapy. In HER2 negative breast cancers, patients with low gene expression of |
| | TOPOIIA may derive benefit from anthracycline-based therapy. |
| TOPO1 | Topoisomerase I is an enzyme that alters the supercoiling of double-stranded DNA. |
| | TOPOI acts by transiently cutting one strand of the DNA to relax the coil and |
| | extend the DNA molecule. Higher expression of TOPOI has been associated with |
| | response to TOPOI inhibitors including irinotecan and topotecan. |
| TS | Thymidylate synthase (TS) is an enzyme involved in DNA synthesis that generates |
| | thymidine monophosphate (dTMP), which is subsequently phosphorylated to |
| | thymidine triphosphate for use in DNA synthesis and repair. Low levels of TS are |
| | predictive of response to fluoropyrimidines and other folate analogues. |
| TUBB3 | Class III β-Tubulin (TUBB3) is part of a class of proteins that provide the |
| | framework for microtubules, major structural components of the cytoskeleton. Due |
| | to their importance in maintaining structural integrity of the cell, microtubules are |
| | ideal targets for anti-cancer agents. Low expression of TUBB3 is associated with |
| | potential clinical benefit to taxanes and vinca alkaloids in certain tumor types. |
| VEGFR2 | VEGFR2, vascular endothelial growth factor 2, is one of three main subtypes of |
| | VEGFR. This protein is an important signaling protein in angiogenesis. Evidence |
| | suggests that increased levels of VEGFR2 may be predictive of response to anti- |
| | angiogenic drugs. |

Tables 7, 9, 11, 13, 15, 17 and 21 present views of the information that can be gathered and reported for the MI and MI Plus molecular profiles. Profiles for various lineages are indicated by the table headers. Modifications made dependent on cancer lineage are indicated as appropriate. The columns headed “Agent/Biomarker Status Reported” provide either candidate agents (e.g., drugs) or biomarker status to be included in the report. Where agents are indicated, the association of the agent with the indicated biomarker is included in the report. Where a status is indicated (e.g., mutational status, protein expression status, gene copy number status), the biomarker status is indicated in the report instead of drug associations. The candidate agents may comprise those undergoing clinical trials, as indicated. Platform abbreviations are as used throughout the application, e.g., IHC: immunohistochemistry; FISH: fluorescent in situ hybridication; CISH: colorimetric in situ hybridization; NGS: next generation sequencing; PCR: polymerase chain reaction.

In an embodiment, the invention provides molecular intelligence (MI) profiles for an ovarian cancer comprising assessment of one or more, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57 or 58, of: ABL1, AKT1, ALK, APC, AR, ATM, BRAF, CDH1, cKIT, cMET, CSF1R, CTNNB1, EGFR, ER, ERBB2, ERBB4, FBXW7, FGFR1, FGFR2, FLT3, GNA11, GNAQ, GNAS, HER2, HNF1A, HRAS, IDH1, JAK2, JAK3, KDR (VEGFR2), KRAS, MGMT, MLH1, MPL, NOTCH1, NPM1, NRAS, PDGFRA, PGP, PIK3CA, PR, PTEN, PTPN11, RB1, RET, RRM1, SMAD4, SMARCB1, SMO, SPARC, STK11, TLE3, TOP2A, TOPO1, TP53, TS, TUBB3, VHL. The invention further provides a method of selecting a candidate treatment for an ovarian cancer comprising assessment of one or more members of the ovarian cancer molecular profile using one or more molecular profiling technique presented herein, e.g., ISH (e.g., FISH, CISH), IHC, RT-PCR, expression array, mutation analysis (e.g., NextGen sequencing, Sanger sequencing, pyrosequencing, Fragment analysis (FA, e.g., RFLP), PCR), etc. In one embodiment, ISH is used to assess one or more, e.g., 1 or 2, of: cMET, HER2. Any useful ISH technique can be used. For example, FISH can be used to assess cMET and/or HER2; or CISH can be used to assess cMET and/or HER2. In an embodiment, protein analysis such as IHC is used to assess one or more, e.g., 1, 2, 3, 4, 5, 6, 7, 8, 9, 10, 11, 12, 13, 14, 15 or 16 of: AR, cMET, ER, HER2, MGMT, PGP, PR, PTEN, RRM1, SPARCm, SPARCp, TLE3, TOP2A, TOPO1, TS, TUBB3. “m” and “p” as in SPARC (m/p) refer to IHC performed with monoclonal (“m”) or polyclonal (“p”) primary antibodies. In some embodiments, sequence analysis is used to assess one or more, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44 or 45 of: ABL1, AKT1, ALK, APC, ATM, BRAF, CDH1, cKIT, cMET, CSF1R, CTNNB1, EGFR, ERBB2, ERBB4, FBXW7, FGFR1, FGFR2, FLT3, GNA11, GNAQ, GNAS, HNF1A, HRAS, IDH1, JAK2, JAK3, KDR (VEGFR2), KRAS, MLH1, MPL, NOTCH1, NPM1, NRAS, PDGFRA, PIK3CA, PTEN, PTPNI 1, RB1, RET, SMAD4, SMARCB1, SMO, STKI 1, TP53, VHL. For example, the sequence analysis can be performed on one or more, e.g., 1, 2, 3, 4, 5, 6, 7, 8, 9, 10, 11, 12, 13 or 14 of ABL1, APC, BRAF, EGFR, FLT3, GNAQ, IDH1, JAK2, cKIT, KRAS, MPL, NRAS, PDGFRA, VHL. The sequence analysis can also be performed on one or more, e.g., 1, 2, 3, 4, 5, 6, 7, 8, 9, 10, 11, 12, 13, 14 or 15 of ABL1, APC, BRAF, EGFR, FLT3, GNAQ, IDH1, JAK2, cKIT, KRAS, MPL, NPM1, NRAS, PDGFRA, VHL. The sequencing may be performed using Next Generation sequencing technology or other technologies as described herein. The molecular profile can be based on assessing the biomarkers as illustrated in FIGS. 33C-D or Table 7 below.

In an embodiment, the invention provides a molecular intelligence (MI) profile for an ovarian cancer comprising analysis of the biomarkers in FIG. 33C, which may be assessed as indicated in the paragraph above and/or as in FIG. 33C or Table 7 below. For example, the MI profile for ovarian cancer may comprise: 1) ISH to assess one or more, e.g., 1 or 2, of: cMET, HER2; 2) IHC to assess one or more, e.g., 1, 2, 3, 4, 5, 6, 7, 8, 9, 10, 11, 12, 13, 14, 15 or 16 of: AR, cMET, ER, HER2, MGMT, PGP, PR, PTEN, RRM1, SPARCm, SPARCp, TLE3, TOP2A, TOPO1, TS, TUBB3; and/or 3) sequence analysis to assess one or more, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33 or 34 of: ABL1, AKT1, ALK, APC, ATM, BRAF, cKIT, cMET, CSF1R, CTNNB1, EGFR, ERBB2, FGFR1, FGFR2, FLT3, GNA11, GNAQ, GNAS, HRAS, IDH1, JAK2, KDR (VEGFR2), KRAS, MLH1, MPL, NOTCH1, NRAS, PDGFRA, PIK3CA, PTEN, RET, SMO, TP53, VHL. In another embodiment, the invention provides a molecular intelligence (MI) PLUS profile for an ovarian cancer comprising analysis of the biomarkers in the molecular intelligence (MI) profile and the additional biomarker in FIG. 33D, i.e., 1, 2, 3, 4, 5, 6, 7, 8, 9, 10 or 11 of CDH1, ERBB4, FBXW7, HNF1A, JAK3, NPM1, PTPN11, RB1, SMAD4, SMARCB1 and STK11, which may be assessed as indicated this paragraph and/or as in FIG. 33D or Table 7 below. The invention further provides a report comprising results of the ovarian cancer molecular profiling and corresponding candidate treatments that are identified as likely beneficial or likely not beneficial, as further described herein.

Table 7 presents a view of the information that is reported for the ovarian cancer molecular intelligence molecular profiles. The columns headed “Agent/Biomarker Expression Reported” provide either candidate agents (e.g., drugs) or biomarker status to be included in the report. Where agents are indicated, the association of the agent with the indicated biomarker is included in the report. Where a status is indicated (e.g., mutational status, protein expression status, gene copy number status), the biomarker status is indicated in the report instead of drug associations. The candidate agents may comprise those undergoing clinical trials, as indicated. The ovarian cancer profiles provide standard of care therapies for ovarian cancer according to the NCCN guidelines as well as additional non-standard candidate therapies for treating the cancer. As will be evident to one of skill, the same biomarkers in Table 7 can be assessed using the indicated methodology for both MI and MI Plus molecular profiling.

TABLE 7
Molecular Profile and Report Parameters: Ovarian Cancer

| Agent(s)/Biomarker Status Reported | Biomarker | Platform |
|---|---|---|
| docetaxel, paclitaxel, nab-paclitaxel | TUBB3 | IHC |
| | PgP | IHC |
| | SPARCm | IHC |
| | SPARCp | IHC |
| irinotecan, topotecan | TOPO1 | IHC |
| gemcitabine | RRM1 | IHC |
| doxorubicin, liposomal-doxorubicin, | HER2 | FISH/CISH |
| epirubicin | TOP2A | IHC |
| | PgP | IHC |
| fulvestrant, tamoxifen, letrozole, | ER | IHC |
| anastrozole | | |
| megestrol acetate, leuprolide | ER | IHC |
| | PR | IHC |
| pemetrexed, capecitabine, fluorouracil | TS | IHC |
| trastuzumab, pertuzumab, T-DM1, | HER2 | IHC, |
| clinical trials | | FISH/CISH |
| everolimus, temsirolimus, clinical trials | PIK3CA | NGS |
| protein expression status | AR | IHC |
| | TLE3 | IHC |
| imatinib | cKIT | NGS |
| | PDGFRA | NGS |
| temozolomide, dacarbazine | MGMT | IHC |
| vandetanib | RET | NGS |
| clinical trials | PTEN | IHC |
| clinical trials | cMET | IHC, |
| | | FISH/CISH |
| clinical trials | VHL | NGS |
| clinical trials | PTEN | NGS |
| clinical trials | KRAS | NGS |
| clinical trials | IDH1 | NGS |
| clinical trials | BRAF | NGS |
| clinical trials | NRAS | NGS |
| clinical trials | ABL1 | NGS |
| clinical trials | AKT1 | NGS |
| clinical trials | ALK | NGS |
| clinical trials | APC | NGS |
| clinical trials | ATM | NGS |
| clinical trials | CSF1R | NGS |
| clinical trials | CTNNB1 | NGS |
| clinical trials | EGFR | NGS |
| clinical trials | ERBB2 | NGS |
| | (HER2) | |
| clinical trials | FGFR1 | NGS |
| clinical trials | FGFR2 | NGS |
| clinical trials | FLT3 | NGS |
| clinical trials | GNAQ | NGS |
| clinical trials | GNA11 | NGS |
| clinical trials | GNAS | NGS |
| clinical trials | HRAS | NGS |
| clinical trials | JAK2 | NGS |
| clinical trials | KDR | NGS |
| | (VEGFR2) | |
| clinical trials | cMET | NGS |
| clinical trials | MLH1 | NGS |
| clinical trials | MPL | NGS |
| clinical trials | NOTCH1 | NGS |
| clinical trials | SMO | NGS |
| clinical trials | TP53 | NGS |

The invention further provides a set of biomarker—treatment association rules for an ovarian cancer, wherein the rules comprise a predicted likelihood of benefit or lack of benefit of a certain treatment for the cancer given an assessment of one or more biomarker. The associations/rules for an ovarian cancer may comprise those presented in Table 8. Tables 10, 12, 14, 16, 18, 19, 20 and 22 are interpreted similarly. In the tables, the class of drug and illustrative drugs of the indicated class are indicated in the columns “Class of Drugs” and “Drugs,” respectively. The columns headed “Biomarker Result” illustrate illustrative methods of profiling the indicated biomarkers, wherein the results are generally true (“T”) or false (“F”), “Any,” or “No Data.” The data can also be labeled “Equivocal,” “Equivocal Low,” or “Equivocal High,” e.g., for IHC where the observed expression level is near or at the threshold set to determine whether a protein is under-expressed, over-expressed, or expressed at normal levels. For mutations, in some cases a particular mutation (e.g., BRAF V600E or V600K) or region/mutational hotspot is called out (e.g., c-KIT exon11 or exon13). In some cases, a particular mutation is called out from others in the “Biomarker Result.” For example, in the case of cKIT, the V654A mutation or mutations in exon 14, exon 17, or exon 18 are called out in the rules for the tyrosine kinase inhibitor (“TKI”) imatinib. Similarly, in the case of PDGFRA mutations, the PDGFRA D842V mutation may be called out in the tables apart from other PDGFRA mutations. In the case of the taxanes paclitaxel, docetaxel, nab-paclitaxel, certain biomarker results only implicate the likely benefit or not of nab-paclitaxel while others implicate the likely benefit or not of all of paclitaxel, docetaxel, and nab-paclitaxel. Such determinations can be based on the available evidence. One of skill will appreciate that alternative methods can be used to analyze the biomarkers as appropriate. For example, sequencing analysis performed by Next Generation methodology could also be performed by Sanger sequencing or other forms of sequence analysis method such as those described herein or known in the art that yield similar biological information (e.g., an expression or mutation status). The biomarker results combine to predict a benefit or lack of benefit from treatment with the indicated candidate drugs. As an example in Table 8, consider that PIK3CA exon20 is mutated as determined by sequencing (PIK3CA Mutated exon20=T), then the mTOR inhibitor agents everolimus and/or temsirolimus are predicted to have treatment benefit (Overall Benefit=T). However, if PIK3CA exon20 mutation is determined to be false (“F”) or is not determined (“No Data”), then the overall benefit of the mTOR inhibitors is indeterminate. As another example in Table 8, consider that the sample is determined to be ER positive by IHC. In such case, overall benefit from the hormonal agents leuprolide and/or megestrol acetate is expected to be likely (i.e., true or “T”). These results are independent of the status of PR as also determined by IHC. If ER is determined to not be overexpressed (i.e., false “F”) or no data is available, and PR is determined to be positive by IHC, then overall benefit from the hormonal agents leuprolide and/or megestrol acetate is also expected to be likely (i.e., true or “T”). If neither ER nor PR are expressed (i.e., ER Positive=false (“F”) and PR Positive=false (“F”)), then overall benefit from the hormonal agents leuprolide and/or megestrol acetate is expected to be not likely (i.e., false or “F”). The expected overall benefit from the hormonal agents leuprolide and/or megestrol acetate is indeterminate (i.e., “Indet.”) in either of the following situations: 1) ER is not expressed or data is unavailable (i.e., ER Positive=“No Data”) and data is unavailable for PR (i.e., PR Positive=“No Data”); or 2) data is unavailable for ER (i.e., ER Positive=“No Data”) and PR is not expressed (i.e., PR Positive=“F”).

Abbreviations used in Tables 8, 10, 12, 14, 16, 18, 19, 20 and 22 include: tyrosine kinase inhibitor (“TKI”); Sequencing (“Seq.”); Indeterminate (“Indet.”); True (“T”); False (“F”).

TABLE 8
Rules for Ovarian Cancer Biomarker-Drug Associations

| | | Biomarker | Biomarker | Biomarker | Biomarker | Overall |
|---|---|---|---|---|---|---|
| Class of Drugs | Drugs | Result | Result | Result | Result | benefit |
| Topo1 | irinotecan, | TOPO1 | | | | Overall |
| inhibitors | topotecan | Positive | | | | benefit |
| | | (IHC) | | | | |
| | | T | | | | T |
| | | F | | | | F |
| | | No Data | | | | Indet. |
| Antimetabolites | gemcitabine | RRM1 | | | | Overall |
| | | Negative | | | | benefit |
| | | (IHC) | | | | |
| | | T | | | | T |
| | | F | | | | F |
| | | No Data | | | | Indet. |
| Hormonal | tamoxifen, | ER | | | | Overall |
| Agents | fulvestrant, | Positive | | | | Benefit |
| | letrozole, | (IHC) | | | | |
| | anastrozole | | | | | |
| | | T | | | | T |
| | | F | | | | F |
| | | No Data | | | | Indet. |
| Hormonal | leuprolide, | ER | PR | | | Overall |
| Agents | megestrol | Positive | Positive | | | Benefit |
| | acetate | (IHC) | (IHC) | | | |
| | | T | Any | | | T |
| | | F or No | T | | | T |
| | | Data | | | | |
| | | F | F | | | F |
| | | F or No | No Data | | | Indet. |
| | | Data | | | | |
| | | No Data | F | | | Indet. |
| Antimetabolites | fluorouracil, | TS | | | | Overall |
| | capecitabine, | Negative | | | | benefit |
| | pemetrexed | (IHC) | | | | |
| | | T | | | | T |
| | | F | | | | F |
| | | No Data | | | | Indet. |
| Alkylating | temozolomide, | MGMT | | | | Overall |
| agents | dacarbazine | Negative | | | | benefit |
| | | (IHC) | | | | |
| | | T | | | | T |
| | | F | | | | F |
| | | No Data | | | | Indet. |
| Monoclonal | trastuzumab, | HER2 | HER2 | | | Overall |
| antibodies | pertuzumab, | Positive | Amplified | | | Benefit |
| (Her2- | ado- | (IHC) | (ISH) | | | |
| Targeted) | trastuzumab | | | | | |
| | emtansine (T- | | | | | |
| | DM1) | | | | | |
| | | T | Any | | | T |
| | | F, | T or | | | T |
| | | Equivocal | Equivocal | | | |
| | | or No Data | High | | | |
| | | F or | F or | | | F |
| | | Equivocal | Equivocal | | | |
| | | | Low | | | |
| | | F or | No Data | | | Indet. |
| | | Equivocal | | | | |
| | | No Data | F, | | | Indet. |
| | | | Equivocal | | | |
| | | | Low or No | | | |
| | | | Data | | | |
| mTOR | everolimus, | PIK3CA | | | | Overall |
| inhibitors | temsirolimus | exon20 | | | | Benefit |
| | | (Seq.) | | | | |
| | | T | | | | T |
| | | F or No | | | | Indet. |
| | | Data | | | | |
| TKI | imatinib | c-KIT | PDGFRA | | | Overall |
| | | exon11 \| | exon 12 \| | | | Benefit |
| | | exon13 | exon 14 \| | | | |
| | | (Seq.) | exon 18 | | | |
| | | | (Seq.) | | | |
| | | Any | D842V | | | F |
| | | V654A | Any | | | F |
| | | T | Any other | | | T |
| | | F, exon 14, | T | | | T |
| | | exon 17, | | | | |
| | | exon 18 or | | | | |
| | | No Data | | | | |
| | | F, exon 14, | F or No | | | Indet. |
| | | exon 17, | Data | | | |
| | | exon 18 or | | | | |
| | | No Data | | | | |
| TKI | crizotinib | ALK | ROS1 | | | Overall |
| | | Positive | Positive | | | Benefit |
| | | (ISH) | (ISH) | | | |
| | | T | Any | | | T |
| | | F or No | T | | | T |
| | | Data | | | | |
| | | F | F or No | | | F |
| | | | Data | | | |
| | | No Data | F or No | | | Indet. |
| | | | Data | | | |
| Anthracyclines | doxorubicin, | TOP2A | Her2 | TOP2A | PGP | Overall |
| and related | liposomal- | Amplified | Amplified | Positive | Positive | Benefit |
| substances | doxorubicin, | (ISH) | (ISH) | (IHC) | (IHC) | |
| | epirubicin | | | | | |
| | | T | Any | Any | Any | T |
| | | F or No | T or | Any | Any | T |
| | | Data | Equivocal | | | |
| | | | High | | | |
| | | F or No | F, | T | Any | T |
| | | Data | Equivocal | | | |
| | | | Low or No | | | |
| | | | Data | | | |
| | | F | F, | F or No | Any | F |
| | | | Equivocal | Data | | |
| | | | Low or No | | | |
| | | | Data | | | |
| | | No Data | F or | F or No | Any | F |
| | | | Equivocal | Data | | |
| | | | Low | | | |
| | | No Data | No Data | F | Any | F |
| | | No Data | No Data | No Data | T | F |
| | | No Data | No Data | No Data | F | T |
| | | No Data | No Data | No Data | No Data | Indet. |
| TKI (RET- | vandetanib | RET | | | | Overall |
| targeted) | | Mutated | | | | benefit |
| | | (Seq.) | | | | |
| | | T | | | | T |
| | | F or No | | | | Indet. |
| | | Data | | | | |
| Taxanes | paclitaxel, | SPARC | SPARC | TUBB3 | PGP | Overall |
| | docetaxel, nab- | Positive | Positive | Positive | Positive | Benefit |
| | paclitaxel | (Mono | (Poly IHC) | (IHC) | (IHC) | |
| | | IHC) | | | | |
| | nab-paclitaxel | T | Any | T or No | Any | T |
| | | | | Data | | |
| | paclitaxel, | T | Any | F | Any | T |
| | docetaxel, nab- | | | | | |
| | paclitaxel | | | | | |
| | nab-paclitaxel | F or No | T | T or No | Any | T |
| | | Data | | Data | | |
| | paclitaxel, | F or No | T | F | Any | T |
| | docetaxel, nab- | Data | | | | |
| | paclitaxel | | | | | |
| | paclitaxel, | F or No | F or No | T | Any | F |
| | docetaxel, nab- | Data | Data | | | |
| | paclitaxel | | | | | |
| | paclitaxel, | F or No | F or No | F | Any | T |
| | docetaxel, nab- | Data | Data | | | |
| | paclitaxel | | | | | |
| | nab-paclitaxel | F | F or No | No Data | Any | F |
| | | | Data | | | |
| | nab-paclitaxel | No Data | F | No Data | Any | F |
| | paclitaxel, | No Data | No Data | No Data | Any | Indet. |
| | docetaxel, nab- | | | | | |
| | paclitaxel | | | | | |

In an aspect, the invention provides molecular intelligence (MI) profiles for breast cancer comprising assessment of one or more, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57 or 58, of: ABL1, AKT1, ALK, APC, AR, ATM, BRAF, CDH1, cKIT, cMET, CSF1R, CTNNB1, EGFR, ER, ERBB2, ERBB4, FBXW7, FGFR1, FGFR2, FLT3, GNA11, GNAQ, GNAS, HER2, HNFIA, HRAS, IDH1, JAK2, JAK3, KDR (VEGFR2), KRAS, MGMT, MLH1, MPL, NOTCH1, NPM1, NRAS, PDGFRA, PGP, PIK3CA, PR, PTEN, PTPN11, RB1, RET, RRM1, SMAD4, SMARCB1, SMO, SPARC, STK11, TLE3, TOP2A, TOPO1, TP53, TS, TUBB3, VHL. The invention further provides a method of selecting a candidate treatment for a breast cancer comprising assessment of one or more members of the breast cancer molecular profile using one or more molecular profiling technique presented herein, e.g., ISH (e.g., FISH, CISH), IHC, RT-PCR, expression array, mutation analysis (e.g., NextGen sequencing, Sanger sequencing, pyrosequencing, Fragment analysis (FA, e.g., RFLP), PCR), etc. In one embodiment, ISH is used to assess one or more, e.g., 1, 2 or 3, of: cMET, HER2, TOP2A. Any useful ISH technique can be used. For example, FISH can be used to assess TOP2A and CISH can be used to assess HER2 and cMET. CISH can also be used to assess TOP2A. As desired, FISH can be used to assess HER2 and/or cMET. In an embodiment, protein analysis such as IHC is used to assess one or more, e.g., 1, 2, 3, 4, 5, 6, 7, 8, 9, 10, 11, 12, 13, 14, 15 or 16 of: AR, cMET, ER, HER2, MGMT, PGP, PR, PTEN, RRM1, SPARCm, SPARCp, TLE3, TOPO1, TS, TUBB3. “m” and “p” as in SPARC (m/p) refer to IHC performed with monoclonal (“m”) or polyclonal (“p”) primary antibodies. In some embodiments, sequence analysis is used to assess one or more, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44 or 45 of: ABL1, AKT1, ALK, APC, ATM, BRAF, CDH1, cKIT, cMET, CSF1R, CTNNB1, EGFR, ERBB2, ERBB4, FBXW7, FGFR1, FGFR2, FLT3, GNA11, GNAQ, GNAS, HNF1A, HRAS, IDH1, JAK2, JAK3, KDR (VEGFR2), KRAS, MLH1, MPL, NOTCH1, NPM1, NRAS, PDGFRA, PIK3CA, PTEN, PTPN11, RB1, RET, SMAD4, SMARCB1, SMO, STK11, TP53, VHL. For example, the sequence analysis can be performed on one or more, e.g., 1, 2, 3, 4, 5, 6, 7, 8, 9, 10, 11, 12, 13 or 14 of ABL1, APC, BRAF, EGFR, FLT3, GNAQ, IDH1, JAK2, cKIT, KRAS, MPL, NRAS, PDGFRA, VHL. The sequence analysis can also be performed on one or more, e.g., 1, 2, 3, 4, 5, 6, 7, 8, 9, 10, 11, 12, 13, 14 or 15 of ABL1, APC, BRAF, EGFR, FLT3, GNAQ, IDH1, JAK2, cKIT, KRAS, MPL, NPM1, NRAS, PDGFRA, VHL. The sequencing may be performed using Next Generation sequencing technology or other technologies as described herein. The molecular profile can be based on assessing the biomarkers as illustrated in FIGS. 33K-L or Table 9 below.

In an embodiment, the invention provides a molecular intelligence (MI) profile for a breast cancer comprising analysis of the biomarkers in FIG. 33K or Table 9 below. For example, the MI profile for breast cancer may comprise: 1) ISH to assess one or more, e.g., 1, 2 or 3, of: cMET, HER2, TOP2A; 2) IHC to assess one or more, e.g., 1, 2, 3, 4, 5, 6, 7, 8, 9, 10, 11, 12, 13, 14, 15 or 16 of: AR, cMET, ER, HER2, MGMT, PGP, PR, PTEN, RRM1, SPARCm, SPARCp, TLE3, TOPO1, TS, TUBB3; and/or 3) sequence analysis to assess one or more, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33 or 34 of: ABL1, AKT1, ALK, APC, ATM, BRAF, cKIT, cMET, CSF1R, CTNNB1, EGFR, ERBB2, FGFR1, FGFR2, FLT3, GNA11, GNAQ, GNAS, HRAS, IDH1, JAK2, KDR (VEGFR2), KRAS, MLH1, MPL, NOTCH1, NRAS, PDGFRA, PIK3CA, PTEN, RET, SMO, TP53, VHL. In another embodiment, the invention provides a molecular intelligence (MI) PLUS profile for a breast cancer comprising analysis of the biomarkers in the molecular intelligence (MI) profile and the additional biomarker in FIG. 33L, i.e., 1, 2, 3, 4, 5, 6, 7, 8, 9, 10 or 11 of CDH1, ERBB4, FBXW7, HNF1A, JAK3, NPM1, PTPN11, RB1, SMAD4, SMARCB1 and STK11, which may be assessed as indicated this paragraph and/or as in FIG. 33L or Table 9 below. The invention further provides a report comprising results of the breast cancer molecular profiling and corresponding candidate treatments that are identified as likely beneficial or likely not beneficial, as further described herein.

Table 9 presents a view of the information that is reported for the breast cancer molecular intelligence molecular profiles, which can be interpreted as described for Table 7 above. The biomarker-treatment associations for the molecular profile for breast cancer may comprise those associations in Table 10, which can be interpreted as described for Table 8 above.

TABLE 9
Molecular Profile and Report Parameters: Breast Cancer

| Agent(s)/Biomarker Status Reported | Biomarker | Platform |
|---|---|---|
| fulvestrant, tamoxifen, toremifene; | ER | IHC |
| anastrozole, exemestane, letrozole; | PR | IHC |
| leuprolide, goserelin, megestrol acetate | | |
| trastuzumab | HER2 | IHC; |
| | | FISH/CISH |
| | PTEN | IHC |
| | PIK3CA | NGS |
| lapatinib, pertuzumab, T-DM1, clinical | HER2 | IHC; |
| trials | | FISH/CISH |
| doxorubicin, liposomal-doxorubicin, | TOP2A | FISH/CISH |
| epirubicin | HER2 | FISH/CISH |
| fluorouracil, capecitabine, pemetrexed | TS | IHC |
| docetaxel, paclitaxel, nab-paclitaxel | TLE3 | IHC |
| | PgP | IHC |
| | SPARCm | IHC |
| | SPARCp | IHC |
| gemcitabine | RRM1 | IHC |
| irinotecan | TOPO1 | IHC |
| everolimus, temsirolimus, clinical trials | ER | IHC |
| | HER2 | IHC, |
| | | FISH/CISH |
| | PIK3CA | NGS |
| protein expression status | TUBB3 | IHC |
| imatinib | cKIT | NGS |
| vandetanib | RET | NGS |
| clinical trials | AR | IHC |
| clinical trials | cMET | IHC, |
| | | FISH/CISH |
| clinical trials | BRAF | NGS |
| | KRAS | NGS |
| | NRAS | NGS |
| clinical trials | IDH1 | NGS |
| clinical trials | VHL | NGS |
| clinical trials | PTEN | NGS |
| Clinical Trials | ABL1 | NGS |
| Clinical Trials | AKT1 | NGS |
| Clinical Trials | ALK | NGS |
| Clinical Trials | APC | NGS |
| Clinical Trials | ATM | NGS |
| Clinical Trials | CSF1R | NGS |
| Clinical Trials | CTNNB1 | NGS |
| Clinical Trials | EGFR | NGS |
| Clinical Trials | ERBB2 | NGS |
| | (HER2) | |
| Clinical Trials | FGFR1 | NGS |
| Clinical Trials | FGFR2 | NGS |
| Clinical Trials | FLT3 | NGS |
| Clinical Trials | GNAQ | NGS |
| Clinical Trials | GNA11 | NGS |
| Clinical Trials | GNAS | NGS |
| Clinical Trials | HRAS | NGS |
| Clinical Trials | JAK2 | NGS |
| Clinical Trials | KDR | NGS |
| | (VEGFR2) | |
| Clinical Trials | cMET | NGS |
| Clinical Trials | MLH1 | NGS |
| Clinical Trials | MPL | NGS |
| Clinical Trials | NOTCH1 | NGS |
| Clinical Trials | SMO | NGS |
| Clinical Trials | TP53 | NGS |

TABLE 10
Rules for Breast Cancer Biomarker-Drug Associations

| | | Biomarker | Biomarker | Biomarker | Biomarker | Overall |
|---|---|---|---|---|---|---|
| Drug Class | Drugs | Result | Result | Result | Result | benefit |
| Antimetabolites | gemcitabine | RRM1 | | | | Overall |
| | | Negative | | | | benefit |
| | | (IHC) | | | | |
| | | T | | | | T |
| | | F | | | | F |
| | | No Data | | | | Indet. |
| Antimetabolites | fluorouracil, | TS | | | | Overall |
| | capecitabine, | Negative | | | | benefit |
| | pemetrexed | (IHC) | | | | |
| | | T | | | | T |
| | | F | | | | F |
| | | No Data | | | | Indet. |
| Topo1 | irinotecan | TOPO1 | | | | Overall |
| inhibitors | | Positive | | | | benefit |
| | | (IHC) | | | | |
| | | T | | | | T |
| | | F | | | | F |
| | | No Data | | | | Indet. |
| Hormonal | tamoxifen, | ER | PR | | | Overall |
| Agents | toremifene, | Positive | Positive | | | Benefit |
| | fulvestrant, | (IHC) | (IHC) | | | |
| | letrozole, | | | | | |
| | anastrozole, | | | | | |
| | exemestane, | | | | | |
| | megestrol | | | | | |
| | acetate, | | | | | |
| | leuprolide, | | | | | |
| | goserelin | | | | | |
| | | T | Any | | | T |
| | | F or No | T | | | T |
| | | Data | | | | |
| | | F | F | | | F |
| | | F | No Data | | | Indet. |
| | | No Data | F or No | | | Indet. |
| | | | Data | | | |
| Her2-targeted | lapatinib, | HER2 | HER2 | | | Overall |
| Agents | pertuzumab, | Positive | Amplified | | | Benefit |
| | ado- | (IHC) | (ISH) | | | |
| | trastuzumab | | | | | |
| | emtansine (T- | | | | | |
| | DM1) | | | | | |
| | | T | Any | | | T |
| | | F, | T or | | | T |
| | | Equivocal | Equiviocal | | | |
| | | or No Data | High | | | |
| | | F or | F or | | | F |
| | | Equivocal | Equivocal | | | |
| | | | Low | | | |
| | | F or | No Data | | | Indet. |
| | | Equivocal | | | | |
| | | No Data | F, | | | Indet. |
| | | | Equivocal | | | |
| | | | Low or No | | | |
| | | | Data | | | |
| Anthracyclines | doxorubicin, | TOP2A | HER2 | | | Overall |
| and related | liposomal- | Amplified | Amplified | | | Benefit |
| substances | doxorubicin, | (ISH) | (ISH) | | | |
| | epirubicin | | | | | |
| | | T | Any | | | T |
| | | F or No | T or | | | T |
| | | Data | Equivocal | | | |
| | | | High | | | |
| | | F | F, No Data | | | F |
| | | | or | | | |
| | | | Equivocal | | | |
| | | | Low | | | |
| | | No Data | F or | | | F |
| | | | Equivocal | | | |
| | | | Low | | | |
| | | No Data | No Data | | | Indet. |
| TKI | crizotinib | ALK | ROS1 | | | Overall |
| | | Positive | Positive | | | Benefit |
| | | (ISH) | (ISH) | | | |
| | | T | Any | | | T |
| | | F or No | T | | | T |
| | | Data | | | | |
| | | F | F or No | | | F |
| | | | Data | | | |
| | | No Data | F or No | | | Indet. |
| | | | Data | | | |
| Monoclonal | trastuzumab | HER2 | HER2 | PTEN | PIK3CA | Overall |
| antibodies | | Positive | Amplified | Negative | Mutated \| | Benefit |
| (Her2-Targeted- | | (IHC) | (ISH) | (IHC) | exon20 | |
| trastuzumab) | | | | | (Seq.) | |
| | | T | Any | Any | Any | T |
| | | F, | T or | Any | Any | T |
| | | Equivocal | Equivocal | | | |
| | | or No Data | High | | | |
| | | F or | F or | Any | Any | F |
| | | Equivocal | Equivocal | | | |
| | | | Low | | | |
| | | F or | No Data | Any | Any | Indet. |
| | | Equivocal | | | | |
| | | No Data | F, | Any | Any | Indet. |
| | | | Equivocal | | | |
| | | | Low or No | | | |
| | | | Data | | | |
| Alkylating | temozolomide, | MGMT | | | | Overall |
| agents | dacarbazine | Negative | | | | benefit |
| | | (IHC) | | | | |
| | | T | | | | T |
| | | F | | | | F |
| | | No Data | | | | Indet. |
| mTOR | everolimus, | ER | Her2 | Her2 | PIK3CA | Overall |
| inhibitors | temsirolimus | Positive | Positive | Amplified | exon 20 | Benefit |
| | | T | T | Any | Any | F |
| | | T | F, | T or | Any | F |
| | | | Equivocal | Equivocal | | |
| | | | or No Data | High | | |
| | | T | F, | F, | Any | T |
| | | | Equivocal | Equivocal | | |
| | | | or No Data | Low or No | | |
| | | | | Data | | |
| | | F | Any | Any | Any | F |
| | | No Data | T | Any | Any | F |
| | | No Data | F, | T or | Any | F |
| | | | Equivocal | Equivocal | | |
| | | | or No Data | High | | |
| | | No Data | F, | F, | Any | Indet. |
| | | | Equivocal | Equivocal | | |
| | | | or No Data | Low or No | | |
| | | | | Data | | |
| TKI (RET- | van detanib | RET | | | | Overall |
| targeted) | | Mutated | | | | benefit |
| | | (Seq.) | | | | |
| | | T | | | | T |
| | | F or No | | | | Indet. |
| | | Data | | | | |
| Taxanes | paclitaxel, | SPARC | SPARC | TLE3 | PGP | Overall |
| | docetaxel, nab- | Positive | Positive | Positive | Positive | Benefit |
| | paclitaxel | (Mono | (Poly IHC) | (IHC) | (IHC) | |
| | | IHC) | | | | |
| | paclitaxel, | Any | Any | T | Any | T |
| | docetaxel, nab- | | | | | |
| | paclitaxel | | | | | |
| | nab-paclitaxel | T or F | Any | F or No | Any | T |
| | | | | Data | | |
| | paclitaxel, | F or No | F | F | Any | F |
| | docetaxel, nab- | Data | | | | |
| | paclitaxel | | | | | |
| | nab-paclitaxel | F | F or No | No Data | Any | F |
| | | | Data | | | |
| | paclitaxel, | F | No Data | F | Any | F |
| | docetaxel, nab- | | | | | |
| | paclitaxel | | | | | |
| | nab-paclitaxel | No Data | T | F or No | Any | T |
| | | | | Data | | |
| | nab-paclitaxel | No Data | No Data | F | Any | F |
| | paclitaxel, | No Data | No Data | F | Any | F |
| | docetaxel, nab- | | | | | |
| | paclitaxel | | | | | |
| | paclitaxel, | No Data | No Data | No Data | Any | Indet. |
| | docetaxel, nab- | | | | | |
| | paclitaxel | | | | | |
| TKI | imatinib | c-KIT | PDGFRA | | | Overall |
| | | exon11 \| | exon 12 \| | | | Benefit |
| | | exon13 | exon 14 \| | | | |
| | | (Seq.) | exon 18 | | | |
| | | | (Seq.) | | | |
| | | Any | D842V | | | F |
| | | V654A | Any | | | F |
| | | T | Any other | | | T |
| | | F, exon 14, | T | | | T |
| | | exon 17, | | | | |
| | | exon 18 or | | | | |
| | | No Data | | | | |
| | | F, exon 14, | F or No | | | Indet. |
| | | exon 17, | Data | | | |
| | | exon 18 or | | | | |
| | | No Data | | | | |

In an aspect, the invention provides molecular intelligence (MI) profiles for melanoma comprising assessment of one or more, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57 or 58, of: ABL1, AKT1, ALK, APC, AR, ATM, BRAF, CDH1, cKIT, cMET, CSF1R, CTNNB1, EGFR, ER, ERBB2, ERBB4, FBXW7, FGFR1, FGFR2, FLT3, GNA11, GNAQ, GNAS, HER2, HNF1A, HRAS, IDH1, JAK2, JAK3, KDR (VEGFR2), KRAS, MGMT, MLH1, MPL, NOTCH1, NPM1, NRAS, PDGFRA, PGP, PIK3CA, PR, PTEN, PTPN11, RB1, RET, RRM1, SMAD4, SMARCB1, SMO, SPARC, STK11, TLE3, TOP2A, TOPO1, TP53, TS, TUBB3, VHL. The invention further provides a method of selecting a candidate treatment for a melanoma comprising assessment of one or more members of the melanoma molecular profile using one or more molecular profiling technique presented herein, e.g., ISH (e.g., FISH, CISH), IHC, RT-PCR, expression array, mutation analysis (e.g., NextGen sequencing, Sanger sequencing, pyrosequencing, Fragment analysis (FA, e.g., RFLP), PCR), etc. In one embodiment, ISH is used to assess one or more, e.g., 1 or 2, of: cMET, HER2. Any useful ISH technique can be used. For example, FISH can be used to assess cMET and/or HER2; or CISH can be used to assess cMET and/or HER2. PCR, e.g., the Cobas V600E test, can be used to assess BRAF. In an embodiment, protein analysis such as IHC is used to assess one or more, e.g., 1, 2, 3, 4, 5, 6, 7, 8, 9, 10, 11, 12, 13, 14, 15 or 16, of: AR, cMET, ER, HER2, MGMT, PGP, PR, PTEN, RRM1, SPARCm, SPARCp, TLE3, TOP2A, TOPO1, TS, TUBB3. “m” and “p” as in SPARC (m/p) refer to IHC performed with monoclonal (“m”) or polyclonal (“p”) primary antibodies. In some embodiments, sequence analysis is used to assess one or more, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44 or 45 of: ABL1, AKT1, ALK, APC, ATM, BRAF, CDH1, cKIT, cMET, CSF1R, CTNNB1, EGFR, ERBB2, ERBB4, FBXW7, FGFR1, FGFR2, FLT3, GNA11, GNAQ, GNAS, HNF1A, HRAS, IDH1, JAK2, JAK3, KDR (VEGFR2), KRAS, MLH1, MPL, NOTCH1, NPM1, NRAS, PDGFRA, PIK3CA, PTEN, PTPN11, RB1, RET, SMAD4, SMARCB1, SMO, STK11, TP53, VHL. For example, the sequence analysis can be performed on one or more, e.g., 1, 2, 3, 4, 5, 6, 7, 8, 9, 10, 11, 12, 13 or 14 of ABL1, APC, BRAF, EGFR, FLT3, GNAQ, IDH1, JAK2, cKIT, KRAS, MPL, NRAS, PDGFRA, VHL. The sequence analysis can also be performed on one or more, e.g., 1, 2, 3, 4, 5, 6, 7, 8, 9, 10, 11, 12, 13, 14 or 15 of ABL1, APC, BRAF, EGFR, FLT3, GNAQ, IDH1, JAK2, cKIT, KRAS, MPL, NPM1, NRAS, PDGFRA, VHL. The sequencing may be performed using Next Generation sequencing technology or other technologies as described herein. The molecular profile can be based on assessing the biomarkers as illustrated in FIG. 33E-F or Table 11 below.

In an embodiment, the invention provides a molecular intelligence (MI) profile for a melanoma comprising analysis of the biomarkers FIG. 33E or Table 11 below. For example, the MI profile for melanoma may comprise: 1) ISH to assess one or more, e.g., 1 or 2, of: cMET, HER2; 2) IHC to assess one or more, e.g., 1, 2, 3, 4, 5, 6, 7, 8, 9, 10, 11, 12, 13, 14, 15 or 16 of: AR, cMET, ER, HER2, MGMT, PGP, PR, PTEN, RRM1, SPARCm, SPARCp, TLE3, TOP2A, TOPO1, TS, TUBB3; 3) PCR to assess BRAF; and/or 4) sequence analysis to assess one or more, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33 or 34 of: ABL1, AKT1, ALK, APC, ATM, BRAF, cKIT, eMET, CSF1R, CTNNB1, EGFR, ERBB2, FGFR1, FGFR2, FLT3, GNA11, GNAQ, GNAS, HRAS, IDH1, JAK2, KDR (VEGFR2), KRAS, MLH1, MPL, NOTCH1, NRAS, PDGFRA, PIK3CA, PTEN, RET, SMO, TP53, VHL. In another embodiment, the invention provides a molecular intelligence (MI) PLUS profile for a melanoma comprising analysis of the biomarkers in the molecular intelligence (MI) profile and the additional biomarker in FIG. 33F, i.e., 1, 2, 3, 4, 5, 6, 7, 8, 9, 10 or 11 of CDH1, ERBB4, FBXW7, HNF1A, JAK3, NPM1, PTPN11, RB1, SMAD4, SMARCB1 and STK11, which may be assessed as indicated this paragraph and/or as in FIG. 33F or Table 11 below. The invention further provides a report comprising results of the molecular profiling and corresponding candidate treatments that are identified as likely beneficial or likely not beneficial, as further described herein.

Table 11 presents a view of the information that is reported for the melanoma molecular intelligence molecular profiles, which can be interpreted as described for Table 7 above. The biomarker-treatment associations for the molecular intelligence molecular profiles for melanoma may comprise those associations in Table 12, which can be interpreted as described for Table 8 above.

TABLE 11
Molecular Profile and Report Parameters: Melanoma

| Agent(s)/Biomarker Status Reported | Biomarker | Platform |
|---|---|---|
| vemurafenib, dabrafenib, trametinib* | BRAF | cobas PCR |
| | | NGS |
| temozolomide, dacarbazine | MGMT | IHC |
| imatinib | cKIT | NGS |
| | PDGFRA | NGS |
| everolimus, temsirolimus, clinical | PIK3CA | NGS |
| trials | | |
| protein expression status | AR | IHC |
| | ER | IHC |
| | PR | IHC |
| paclitaxel, docetaxel, nab-paclitaxel | TLE3 | IHC |
| | TUBB3 | IHC |
| | PgP | IHC |
| | SPARCm | IHC |
| | SPARCp | IHC |
| doxorubicin, liposomal-doxorubicin, | HER2 | FISH/CISH |
| epirubicin | TOP2A | IHC |
| | PgP | IHC |
| trastuzumab, lapatinib, pertuzumab, T- | HER2 | IHC, |
| DM1 | | FISH/CISH |
| gemcitabine | RRM1 | IHC |
| irinotecan | TOPO1 | IHC |
| fluorouracil, capecitabine, pemetrexed | TS | IHC |
| vandetanib | RET | NGS |
| clinical trials | PTEN | IHC |
| clinical trials | cMET | IHC, |
| | | FISH/CISH |
| clinical trials | BRAF | cobas PCR |
| clinical trials | | NGS |
| clinical trials | IDH1 | NGS |
| clinical trials | KRAS | NGS |
| clinical trials | NRAS | NGS |
| clinical trials | VHL | NGS |
| clinical trials | PTEN | NGS |
| clinical trials | ABL1 | NGS |
| clinical trials | AKT1 | NGS |
| clinical trials | ALK | NGS |
| clinical trials | APC | NGS |
| clinical trials | ATM | NGS |
| clinical trials | CSF1R | NGS |
| clinical trials | CTNNB1 | NGS |
| clinical trials | EGFR | NGS |
| clinical trials | ERBB2 | NGS |
| | (HER2) | |
| clinical trials | FGFR1 | NGS |
| clinical trials | FGFR2 | NGS |
| clinical trials | FLT3 | NGS |
| clinical trials | GNAQ | NGS |
| clinical trials | GNA11 | NGS |
| clinical trials | GNAS | NGS |
| clinical trials | HRAS | NGS |
| clinical trials | JAK2 | NGS |
| clinical trials | KDR | NGS |
| | (VEGFR2) | |
| clinical trials | cMET | NGS |
| clinical trials | MLH1 | NGS |
| clinical trials | MPL | NGS |
| clinical trials | NOTCH1 | NGS |
| clinical trials | SMO | NGS |
| clinical trials | TP53 | NGS |

*trametinib association will include BRAF by NGS testing for V600K mutations.

TABLE 12
Rules for Melanoma Biomarker-Drug Associations

| | | Biomarker | Biomarker | Biomarker | Biomarker | Biomarker | Overall |
|---|---|---|---|---|---|---|---|
| Class of Drugs | Drugs | Result | Result | Result | Result | Result | benefit |
| Antimetabolites | gemcitabine | RRM1 | | | | | Overall |
| | | Negative | | | | | benefit |
| | | (IHC) | | | | | |
| | | T | | | | | T |
| | | F | | | | | F |
| | | No Data | | | | | Indet. |
| Antimetabolites | fluorouracil, | TS | | | | | Overall |
| | capecitabine, | Negative | | | | | benefit |
| | pemetrexed | (IHC) | | | | | |
| | | T | | | | | T |
| | | F | | | | | F |
| | | No Data | | | | | Indet. |
| Topo1 | irinotecan | TOPO1 | | | | | Overall |
| inhibitors | | Positive | | | | | benefit |
| | | (IHC) | | | | | |
| | | T | | | | | T |
| | | F | | | | | F |
| | | No Data | | | | | Indet. |
| Alkylating | temozolomide, | MGMT | | | | | Overall |
| agents | dacarbazine | Negative | | | | | benefit |
| | | (IHC) | | | | | |
| | | T | | | | | T |
| | | F | | | | | F |
| | | No Data | | | | | Indet. |
| TKI | vemurafenib, | BRAF | BRAF | | | | Overall |
| | dabrafenib, | V600E | mutated \| | | | | Benefit |
| | trametinib | (PCR) | V600E \| | | | | |
| | | | V600K | | | | |
| | | | (Seq.) | | | | |
| | | T | Any | | | | T |
| | | F | Any | | | | F |
| | | No Data | Any | | | | Indet. |
| mTOR | everolimus, | PIK3CA | | | | | Overall |
| inhibitors | temsirolimus | exon20 | | | | | Benefit |
| | | (Seq.) | | | | | |
| | | T | | | | | T |
| | | F or No | | | | | Indet. |
| | | Data | | | | | |
| TKI | imatinib | c-KIT | PDGFRA | | | | Overall |
| | | exon11 \| | exon 12 \| | | | | Benefit |
| | | exon13 | exon 14 \| | | | | |
| | | (Seq.) | exon 18 | | | | |
| | | | (Seq.) | | | | |
| | | Any | D842V | | | | F |
| | | V654A | Any | | | | F |
| | | T | Any other | | | | T |
| | | F, exon 14, | T | | | | T |
| | | exon 17, | | | | | |
| | | exon 18 or | | | | | |
| | | No Data | | | | | |
| | | F, exon 14, | F or No | | | | Indet. |
| | | exon 17, | Data | | | | |
| | | exon 18 or | | | | | |
| | | No Data | | | | | |
| TKI | lapatinib | HER2 | HER2 | | | | Overall |
| | | Positive | Amplified | | | | Benefit |
| | | (IHC) | (ISH) | | | | |
| | | T | Any | | | | T |
| | | F, | T or | | | | T |
| | | Equivocal | Equivocal | | | | |
| | | or No Data | High | | | | |
| | | F or | F or | | | | F |
| | | Equivocal | Equivocal | | | | |
| | | | Low | | | | |
| | | F or | No Data | | | | Indet. |
| | | Equivocal | | | | | |
| | | No Data | F, | | | | Indet. |
| | | | Equivocal | | | | |
| | | | Low or No | | | | |
| | | | Data | | | | |
| Monoclonal | trastuzumab, | HER2 | HER2 | | | | Overall |
| antibodies | pertuzumab, | Positive | Amplified | | | | Benefit |
| (Her2- | ado- | (IHC) | (ISH) | | | | |
| Targeted) | trastuzumab | | | | | | |
| | emtansine (T- | | | | | | |
| | DM1) | | | | | | |
| | | T | Any | | | | T |
| | | F, | T or | | | | T |
| | | Equivocal | Equivocal | | | | |
| | | or No Data | High | | | | |
| | | F or | F or | | | | F |
| | | Equivocal | Equivocal | | | | |
| | | | Low | | | | |
| | | F or | No Data | | | | Indet. |
| | | Equivocal | | | | | |
| | | No Data | F, | | | | Indet. |
| | | | Equivocal | | | | |
| | | | Low or No | | | | |
| | | | Data | | | | |
| Anthracyclines | doxorubicin, | TOP2A | Her2 | TOP2A | PGP | | Overall |
| and related | liposomal- | Amplified | Amplified | Positive | Positive | | Benefit |
| substances | doxorubicin, | (ISH) | (ISH) | (IHC) | (IHC) | | |
| | epirubicin | | | | | | |
| | | T | Any | Any | Any | | T |
| | | F or No | T or | Any | Any | | T |
| | | Data | Equivocal | | | | |
| | | | High | | | | |
| | | F or No | F, | T | Any | | T |
| | | Data | Equivocal | | | | |
| | | | Low or No | | | | |
| | | | Data | | | | |
| | | F | F, | F or No | Any | | F |
| | | | Equivocal | Data | | | |
| | | | Low or No | | | | |
| | | | Data | | | | |
| | | No Data | F, | F | Any | | F |
| | | | Equivocal | | | | |
| | | | Low or No | | | | |
| | | | Data | | | | |
| | | No Data | F or | No Data | Any | | F |
| | | | Equivocal | | | | |
| | | | Low | | | | |
| | | No Data | No Data | No Data | T | | F |
| | | No Data | No Data | No Data | F | | T |
| | | No Data | No Data | No Data | No Data | | Indet. |
| TKI | crizotinib | ALK | ROS1 | | | | Overall |
| | | Positive | Positive | | | | Benefit |
| | | (ISH) | (ISH) | | | | |
| | | T | Any | | | | T |
| | | F or No | T | | | | T |
| | | Data | | | | | |
| | | F | F or No | | | | F |
| | | | Data | | | | |
| | | No Data | F or No | | | | Indet. |
| | | | Data | | | | |
| TKI (RET- | vandetanib | RET | | | | | Overall |
| targeted) | | Mutated | | | | | benefit |
| | | (Seq.) | | | | | |
| | | T | | | | | T |
| | | F or No | | | | | Indet. |
| | | Data | | | | | |
| Taxanes | paclitaxel, | SPARC | SPARC | TLE3 | TUBB3 | PGP | Overall |
| | docetaxel, | Positive | Positive | Positive | Positive | Positive | Benefit |
| | nab-paclitaxel | (Mono | (Poly IHC) | (IHC) | (IHC) | (IHC) | |
| | | IHC) | | | | | |
| | paclitaxel, | Any | Any | T | Any | Any | T |
| | docetaxel, nab- | | | | | | |
| | paclitaxel | | | | | | |
| | nab-paclitaxel | T | Any | F or No | T or No | Any | T |
| | | | | Data | Data | | |
| | paclitaxel, | T | Any | F or No | F | Any | T |
| | docetaxel, nab- | | | Data | | | |
| | paclitaxel | | | | | | |
| | nab-paclitaxel | F or No | T | F or No | T or No | Any | T |
| | | Data | | Data | Data | | |
| | paclitaxel, | F or No | T | F or No | F | Any | T |
| | docetaxel, nab- | Data | | Data | | | |
| | paclitaxel | | | | | | |
| | paclitaxel, | F or No | F | F | T or No | Any | F |
| | docetaxel, nab- | Data | | | Data | | |
| | paclitaxel | | | | | | |
| | paclitaxel, | F or No | F | F or No | F | Any | T |
| | docetaxel, nab- | Data | | Data | | | |
| | paclitaxel | | | | | | |
| | paclitaxel, | F or No | F | No Data | T | Any | F |
| | docetaxel, nab- | Data | | | | | |
| | paclitaxel | | | | | | |
| | nab-paclitaxel | F | F or No | No Data | No Data | Any | F |
| | | | Data | | | | |
| | paclitaxel, | F | No Data | F | T or No | Any | F |
| | docetaxel, nab- | | | | Data | | |
| | paclitaxel | | | | | | |
| | paclitaxel, | F or No | No Data | F or No | F | Any | T |
| | docetaxel, nab- | Data | | Data | | | |
| | paclitaxel | | | | | | |
| | paclitaxel, | F or No | No Data | No Data | T | Any | F |
| | docetaxel, nab- | Data | | | | | |
| | paclitaxel | | | | | | |
| | nab-paclitaxel | No Data | F | No Data | No Data | Any | F |
| | paclitaxel, | No Data | No Data | F | T or No | Any | F |
| | docetaxel, nab- | | | | Data | | |
| | paclitaxel | | | | | | |
| | paclitaxel, | No Data | No Data | No Data | No Data | Any | Indet. |
| | docetaxel, nab- | | | | | | |
| | paclitaxel | | | | | | |

In an aspect, the invention provides molecular intelligence (MI) profiles for uveal melanoma comprising assessment of one or more, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57 or 58, of: ABL1, AKT1, ALK, APC, AR, ATM, BRAF, CDH1, cKIT, cMET, CSF1R, CTNNB1, EGFR, ER, ERBB2, ERBB4, FBXW7, FGFR1, FGFR2, FLT3, GNA11, GNAQ, GNAS, HER2, HNF1A, HRAS, IDH1, JAK2, JAK3, KDR (VEGFR2), KRAS, MGMT, MLH1, MPL, NOTCH1, NPM1, NRAS, PDGFRA, PGP, PIK3CA, PR, PTEN, PTPN11, RB1, RET, RRM1, SMAD4, SMARCB1, SMO, SPARC, STK11, TLE3, TOP2A, TOPO1, TP53, TS, TUBB3, VHL. The invention further provides a method of selecting a candidate treatment for a uveal melanoma comprising assessment of one or more members of the uveal melanoma molecular profile using one or more molecular profiling technique presented herein, e.g., ISH (e.g., FISH, CISH), IHC, RT-PCR, expression array, mutation analysis (e.g., NextGen sequencing, Sanger sequencing, pyrosequencing, Fragment analysis (FA, e.g., RFLP), PCR), etc. In one embodiment, ISH is used to assess one or more, e.g., 1 or 2, of: cMET, HER2. Any useful ISH technique can be used. For example, FISH can be used to assess cMET and/or HER2; or CISH can be used to assess cMET and/or HER2. PCR, e.g., the Cobas V600E test, can be used to assess BRAF. In an embodiment, protein analysis such as IHC is used to assess one or more, e.g., 1, 2, 3, 4, 5, 6, 7, 8, 9, 10, 11, 12, 13, 14, 15 or 16, of: AR, cMET, ER, HER2, MGMT, PGP, PR, PTEN, RRM1, SPARCm, SPARCp, TLE3, TOP2A, TOPO1, TS, TUBB3. “m” and “p” as in SPARC (m/p) refer to IHC performed with monoclonal (“m”) or polyclonal (“p”) primary antibodies. In some embodiments, sequence analysis is used to assess one or more, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44 or 45 of: ABL1, AKT1, ALK, APC, ATM, BRAF, CDH1, cKIT, cMET, CSF1R, CTNNB1, EGFR, ERBB2, ERBB4, FBXW7, FGFR1, FGFR2, FLT3, GNA11, GNAQ, GNAS, HNF1A, HRAS, IDH1, JAK2, JAK3, KDR (VEGFR2), KRAS, MLH1, MPL, NOTCH1, NPM1, NRAS, PDGFRA, PIK3CA, PTEN, PTPN11, RB1, RET, SMAD4, SMARCB1, SMO, STK11, TP53, VHL. For example, the sequence analysis can be performed on one or more, e.g., 1, 2, 3, 4, 5, 6, 7, 8, 9, 10, 11, 12, 13 or 14 of ABL1, APC, BRAF, EGFR, FLT3, GNAQ, IDH1, JAK2, cKIT, KRAS, MPL, NRAS, PDGFRA, VHL. The sequence analysis can also be performed on one or more, e.g., 1, 2, 3, 4, 5, 6, 7, 8, 9, 10, 11, 12, 13, 14 or 15 of ABL1, APC, BRAF, EGFR, FLT3, GNAQ, IDH1, JAK2, cKIT, KRAS, MPL, NPM1, NRAS, PDGFRA, VHL. The sequencing may be performed using Next Generation sequencing technology or other technologies as described herein. The molecular profile can be based on assessing the biomarkers as illustrated in FIG. 33G-H or Table 11.

In an embodiment, the invention provides a molecular intelligence (MI) profile for a uveal melanoma comprising analysis of the biomarkers in FIG. 33G, which may be assessed as indicated in the paragraph above and/or as in FIG. 33G or Table 11. For example, the MI profile for uveal melanoma may comprise: 1) ISH to assess one or more, e.g., 1 or 2, of: cMET, HER2; 2) IHC to assess one or more, e.g., 1, 2, 3, 4, 5, 6, 7, 8, 9, 10, 11, 12, 13, 14, 15 or 16 of: AR, cMET, ER, HER2, MGMT, PGP, PR, PTEN, RRM1, SPARCm, SPARCp, TLE3, TOP2A, TOPO1, TS, TUBB3; 3) PCR to assess BRAF; and/or 4) sequence analysis to assess one or more, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33 or 34 of: ABL1, AKT1, ALK, APC, A™, BRAF, cKIT, cMET, CSF1R, CTNNB1, EGFR, ERBB2, FGFR1, FGFR2, FLT3, GNA11, GNAQ, GNAS, HRAS, IDH1, JAK2, KDR (VEGFR2), KRAS, MLH1, MPL, NOTCH1, NRAS, PDGFRA, PIK3CA, PTEN, RET, SMO, TP53, VHL. In another embodiment, the invention provides a molecular intelligence (MI) PLUS profile for a uveal melanoma comprising analysis of the biomarkers in the molecular intelligence (MI) profile and the additional biomarker in FIG. 33H, i.e., 1, 2, 3, 4, 5, 6, 7, 8, 9, 10 or 11 of CDH1, ERBB4, FBXW7, HNF1A, JAK3, NPM1, PTPN11, RB1, SMAD4, SMARCB1 and STK11, which may be assessed as indicated this paragraph and/or as in FIG. 33H or Table 11 below. The invention further provides a report comprising results of the molecular profiling and corresponding candidate treatments that are identified as likely beneficial or likely not beneficial, as further described herein.

Table 13 presents a view of the information that is reported for the uveal melanoma molecular intelligence molecular profiles, which can be interpreted as described for Table 7 above. The biomarker—treatment associations for the molecular intelligence molecular profiles for uveal melanoma may comprise those associations in Table 14, which can be interpreted as described for Table 8 above.

TABLE 13
Molecular Profile and Report Parameters: Uveal Melanoma

| Agent(s)/Biomarker Status Reported | Biomarker | Platform |
|---|---|---|
| vemurafenib | BRAF | cobas PCR |
| temozolomide, dacarbazine | MGMT | IHC |
| imatinib | cKIT | NGS |
| | PDGFRA | NGS |
| everolimus, temsirolimus, clinical trials | PIK3CA | NGS |
| protein expression status | AR | IHC |
| | ER | IHC |
| | PR | IHC |
| paclitaxel, docetaxel, nab-paclitaxel | TLE3 | IHC |
| | TUBB3 | IHC |
| | PgP | IHC |
| | SPARCm | IHC |
| | SPARCp | IHC |
| doxorubicin, liposomal-doxorubicin, | HER2 | FISH/CISH |
| epirubicin | TOP2A | IHC |
| | PgP | IHC |
| trastuzumab, lapatinib, pertuzumab, T- | HER2 | IHC, |
| DM1 | | FISH/CISH |
| gemcitabine | RRM1 | IHC |
| irinotecan | TOPO1 | IHC |
| fluorouracil, capecitabine, pemetrexed | TS | IHC |
| vandetanib | RET | NGS |
| clinical trials | cMET | IHC, |
| | | FISH/CISH |
| clinical trials | PTEN | IHC |
| clinical trials | IDH1 | NGS |
| clinical trials | BRAF | NGS |
| clinical trials | KRAS | NGS |
| clinical trials | NRAS | NGS |
| clinical trials | GNA11 | NGS |
| clinical trials | VHL | NGS |
| clinical trials | PTEN | NGS |
| clinical trials | ABL1 | NGS |
| clinical trials | AKT1 | NGS |
| clinical trials | ALK | NGS |
| clinical trials | APC | NGS |
| clinical trials | ATM | NGS |
| clinical trials | CSF1R | NGS |
| clinical trials | CTNNB1 | NGS |
| clinical trials | EGFR | NGS |
| clinical trials | ERBB2 | NGS |
| | (HER2) | |
| clinical trials | FGFR1 | NGS |
| clinical trials | FGFR2 | NGS |
| clinical trials | FLT3 | NGS |
| clinical trials | GNAQ | NGS |
| clinical trials | GNAS | NGS |
| clinical trials | HRAS | NGS |
| clinical trials | JAK2 | NGS |
| clinical trials | KDR | NGS |
| | (VEGFR2) | |
| clinical trials | cMET | NGS |
| clinical trials | MLH1 | NGS |
| clinical trials | MPL | NGS |
| clinical trials | NOTCH1 | NGS |
| clinical trials | SMO | NGS |
| clinical trials | TP53 | NGS |

TABLE 14
Rules for Uveal Melanoma Biomarker-Drug Associations

| | | Biomarker | Biomarker | Biomarker | Biomarker | Biomarker | Overall |
|---|---|---|---|---|---|---|---|
| Class of Drugs | Drugs | Result | Result | Result | Result | Result | benefit |
| Antimetabolites | gemcitabine | RRM1 | | | | | Overall |
| | | Negative | | | | | benefit |
| | | (IHC) | | | | | |
| | | T | | | | | T |
| | | F | | | | | F |
| | | No Data | | | | | Indet. |
| Antimetabolites | fluorouracil, | TS Negative | | | | | Overall |
| | capecitabine, | (IHC) | | | | | benefit |
| | pemetrexed | | | | | | |
| | | T | | | | | T |
| | | F | | | | | F |
| | | No Data | | | | | Indet. |
| Topo1 inhibitors | irinotecan | TOPO1 | | | | | Overall |
| | | Positive | | | | | benefit |
| | | (IHC) | | | | | |
| | | T | | | | | T |
| | | F | | | | | F |
| | | No Data | | | | | Indet. |
| Alkylating | temozolomide, | MGMT | | | | | Overall |
| agents | dacarbazine | Negative | | | | | benefit |
| | | (IHC) | | | | | |
| | | T | | | | | T |
| | | F | | | | | F |
| | | No Data | | | | | Indet. |
| TKI | vemurafenib | BRAF | BRAF | | | | Overall |
| | | V600E | mutated \| | | | | Benefit |
| | | (PCR) | V600E \| | | | | |
| | | | V600K | | | | |
| | | | (Seq.) | | | | |
| | | T | Any | | | | T |
| | | F | Any | | | | F |
| | | No Data | Any | | | | Indet. |
| mTOR | everolimus, | PIK3CA | | | | | Overall |
| inhibitors | temsirolimus | exon20 | | | | | Benefit |
| | | (Seq.) | | | | | |
| | | T | | | | | T |
| | | F or No Data | | | | | Indet. |
| TKI | imatinib | c-KIT | PDGFRA | | | | Overall |
| | | exon11 \| | exon 12 \| | | | | Benefit |
| | | exon13 | exon 14 \| | | | | |
| | | (Seq.) | exon 18 | | | | |
| | | | (Seq.) | | | | |
| | | Any | D842V | | | | F |
| | | V654A | Any | | | | F |
| | | T | Any other | | | | T |
| | | F, exon 14, | T | | | | T |
| | | exon 17, | | | | | |
| | | exon 18 or | | | | | |
| | | No Data | | | | | |
| | | F, exon 14, | F or No | | | | Indet. |
| | | exon 17, | Data | | | | |
| | | exon 18 or | | | | | |
| | | No Data | | | | | |
| TKI | lapatinib | HER2 | HER2 | | | | Overall |
| | | Positive | Amplified | | | | Benefit |
| | | (IHC) | (ISH) | | | | |
| | | T | Any | | | | T |
| | | F, Equivocal | T or | | | | T |
| | | or No Data | Equivocal | | | | |
| | | | High | | | | |
| | | F or | F or | | | | F |
| | | Equivocal | Equivocal | | | | |
| | | | Low | | | | |
| | | F or | No Data | | | | Indet. |
| | | Equivocal | | | | | |
| | | No Data | F, Equivocal | | | | Indet. |
| | | | Low or No | | | | |
| | | | Data | | | | |
| Monoclonal | trastuzumab, | HER2 | HER2 | | | | Overall |
| antibodies | pertuzumab, | Positive | Amplified | | | | Benefit |
| (Her2-Targeted) | ado-trastuzumab | (IHC) | (ISH) | | | | |
| | emtansine (T- | | | | | | |
| | DM1) | | | | | | |
| | | T | Any | | | | T |
| | | F, Equivocal | T or | | | | T |
| | | or No Data | Equivocal | | | | |
| | | | High | | | | |
| | | F or | F or | | | | F |
| | | Equivocal | Equivocal | | | | |
| | | | Low | | | | |
| | | F or | No Data | | | | Indet. |
| | | Equivocal | | | | | |
| | | No Data | F, Equivocal | | | | Indet. |
| | | | Low or No | | | | |
| | | | Data | | | | |
| TKI | crizotinib | ALK | ROS1 | | | | Overall |
| | | Positive | Positive | | | | Benefit |
| | | (ISH) | (ISH) | | | | |
| | | T | Any | | | | T |
| | | F or No Data | T | | | | T |
| | | F | F or No | | | | F |
| | | | Data | | | | |
| | | No Data | F or No | | | | Indet. |
| | | | Data | | | | |
| Anthracyclines | doxorubicin, | TOP2A | Her2 | TOP2A | PGP | | Overall |
| and related | liposomal- | Amplified | Amplified | Positive | Positive | | Benefit |
| substances | doxorubicin, | (ISH) | (ISH) | (IHC) | (IHC) | | |
| | epirubicin | | | | | | |
| | | T | Any | Any | Any | | T |
| | | F or No Data | T or | Any | Any | | T |
| | | | Equivocal | | | | |
| | | | High | | | | |
| | | F or No Data | F, Equivocal | T | Any | | T |
| | | | Low or No | | | | |
| | | | Data | | | | |
| | | F | F, Equivocal | F or No | Any | | F |
| | | | Low or No | Data | | | |
| | | | Data | | | | |
| | | No Data | F, Equivocal | F | Any | | F |
| | | | Low or No | | | | |
| | | | Data | | | | |
| | | No Data | F or | No Data | Any | | F |
| | | | Equivocal | | | | |
| | | | Low | | | | |
| | | No Data | No Data | No Data | T | | F |
| | | No Data | No Data | No Data | F | | T |
| | | No Data | No Data | No Data | No Data | | Indet. |
| TKI (RET- | vandetanib | RET | | | | | Overall |
| targeted) | | Mutated | | | | | benefit |
| | | (Seq.) | | | | | |
| | | T | | | | | T |
| | | F or No Data | | | | | Indet. |
| Taxanes | paclitaxel, | SPARC | SPARC | TLE3 | TUBB3 | PGP | Overall |
| | docetaxel, nab- | Positive | Positive | Positive | Positive | Positive | Benefit |
| | paclitaxel | (Mono IHC) | (Poly IHC) | (IHC) | (IHC) | (IHC) | |
| | paclitaxel, | Any | Any | T | Any | Any | T |
| | docetaxel, nab- | | | | | | |
| | paclitaxel | | | | | | |
| | nab-paclitaxel | T | Any | F or No | T or No | Any | T |
| | | | | Data | Data | | |
| | paclitaxel, | T | Any | F or No | F | Any | T |
| | docetaxel, nab- | | | Data | | | |
| | paclitaxel | | | | | | |
| | nab-paclitaxel | F or No Data | T | F or No | T or No | Any | T |
| | | | | Data | Data | | |
| | paclitaxel, | F or No Data | T | F or No | F | Any | T |
| | docetaxel, nab- | | | Data | | | |
| | paclitaxel | | | | | | |
| | paclitaxel, | F or No Data | F | F | T or No | Any | F |
| | docetaxel, nab- | | | | Data | | |
| | paclitaxel | | | | | | |
| | paclitaxel, | F or No Data | F | F or No | F | Any | T |
| | docetaxel, nab- | | | Data | | | |
| | paclitaxel | | | | | | |
| | paclitaxel, | F or No Data | F | No Data | T | Any | F |
| | docetaxel, nab- | | | | | | |
| | paclitaxel | | | | | | |
| | nab-paclitaxel | F | F or No | No Data | No Data | Any | F |
| | | | Data | | | | |
| | paclitaxel, | F | No Data | F | T or No | Any | F |
| | docetaxel, nab- | | | | Data | | |
| | paclitaxel | | | | | | |
| | paclitaxel, | F or No Data | No Data | F or No | F | Any | T |
| | docetaxel, nab- | | | Data | | | |
| | paclitaxel | | | | | | |
| | paclitaxel, | F or No Data | No Data | No Data | T | Any | F |
| | docetaxel, nab- | | | | | | |
| | paclitaxel | | | | | | |
| | nab-paclitaxel | No Data | F | No Data | No Data | Any | F |
| | paclitaxel, | No Data | No Data | F | T or No | Any | F |
| | docetaxel, nab- | | | | Data | | |
| | paclitaxel | | | | | | |
| | paclitaxel, | No Data | No Data | No Data | No Data | Any | Indet. |
| | docetaxel, nab- | | | | | | |
| | paclitaxel | | | | | | |

In an aspect, the invention provides molecular intelligence (MI) profiles for colorectal cancer (CRC) comprising assessment of one or more, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57 or 58, of: ABL1, AKT1, ALK, APC, AR, ATM, BRAF, CDH1, cKIT, cMET, CSF1R, CTNNB1, EGFR, ER, ERBB2, ERBB4, FBXW7, FGFR1, FGFR2, FLT3, GNA11, GNAQ, GNAS, HER2, HNF1A, HRAS, IDH1, JAK2, JAK3, KDR (VEGFR2), KRAS, MGMT, MLH1, MPL, NOTCH1, NPM1, NRAS, PDGFRA, PGP, PIK3CA, PR, PTEN, PTPN11, RB1, RET, RRM1, SMAD4, SMARCB1, SMO, SPARC, STK11, TLE3, TOP2A, TOPO1, TP53, TS, TUBB3, VHL. The invention further provides a method of selecting a candidate treatment for a CRC comprising assessment of one or more members of the CRC molecular profile using one or more molecular profiling technique presented herein, e.g., ISH (e.g., FISH, CISH), IHC, RT-PCR, expression array, mutation analysis (e.g., NextGen sequencing, Sanger sequencing, pyrosequencing, Fragment analysis (FA, e.g., RFLP), PCR), etc. In one embodiment, ISH is used to assess one or more, e.g., 1 or 2, of: cMET, HER2. Any useful ISH technique can be used. For example, FISH can be used to assess cMET and/or HER2; or CISH can be used to assess cMET and/or HER2. In an embodiment, protein analysis such as IHC is used to assess one or more, e.g., 1, 2, 3, 4, 5, 6, 7, 8, 9, 10, 11, 12, 13, 14, 15 or 16, of: AR, cMET, ER, HER2, MGMT, PGP, PR, PTEN, RRM1, SPARCm, SPARCp, TLE3, TOP2A, TOPO1, TS, TUBB3. “m” and “p” as in SPARC (m/p) refer to IHC performed with monoclonal (“m”) or polyclonal (“p”) primary antibodies. In some embodiments, sequence analysis is used to assess one or more, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44 or 45 of: ABL1, AKT1, ALK, APC, ATM, BRAF, CDH1, cKIT, cMET, CSF1R, CTNNB1, EGFR, ERBB2, ERBB4, FBXW7, FGFR1, FGFR2, FLT3, GNA11, GNAQ, GNAS, HNF1A, HRAS, IDH1, JAK2, JAK3, KDR (VEGFR2), KRAS, MLH1, MPL, NOTCH1, NPM1, NRAS, PDGFRA, PIK3CA, PTEN, PTPN11, RB1, RET, SMAD4, SMARCB1, SMO, STK11, TP53, VHL. For example, the sequence analysis can be performed on one or more, e.g., 1, 2, 3, 4, 5, 6, 7, 8, 9, 10, 11, 12, 13 or 14 of ABL1, APC, BRAF, EGFR, FLT3, GNAQ, IDH1, JAK2, cKIT, KRAS, MPL, NRAS, PDGFRA, VHL. The sequence analysis can also be performed on one or more, e.g., 1, 2, 3, 4, 5, 6, 7, 8, 9, 10, 11, 12, 13, 14 or 15 of ABL1, APC, BRAF, EGFR, FLT3, GNAQ, IDH1, JAK2, cKIT, KRAS, MPL, NPM1, NRAS, PDGFRA, VHL. The sequencing may be performed using Next Generation sequencing technology or other technologies as described herein. The molecular profile can be based on assessing the biomarkers as illustrated in FIG. 33M-N or Table 15 below.

In an embodiment, the invention provides a molecular intelligence (MI) profile for a CRC comprising analysis of the biomarkers in FIG. 33M, which may be assessed as indicated in FIG. 33M or Table 15 below. For example, the MI profile for colorectal cancer may comprise: 1) ISH to assess one or more, e.g., 1 or 2, of: cMET, HER2; 2) IHC to assess one or more, e.g., 1, 2, 3, 4, 5, 6, 7, 8, 9, 10, 11, 12, 13, 14, 15 or 16 of: AR, cMET, ER, HER2, MGMT, PGP, PR, PTEN, RRM1, SPARCm, SPARCp, TLE3, TOP2A, TOPO1, TS, TUBB3; and/or 3) sequence analysis to assess one or more, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33 or 34 of: ABL1, AKT1, ALK, APC, ATM, BRAF, cKIT, cMET, CSF1R, CTNNB1, EGFR, ERBB2, FGFR1, FGFR2, FLT3, GNA11, GNAQ, GNAS, HRAS, IDH1, JAK2, KDR (VEGFR2), KRAS, MLH1, MPL, NOTCH1, NRAS, PDGFRA, PIK3CA, PTEN, RET, SMO, TP53, VHL. In another embodiment, the invention provides a molecular intelligence (MI) PLUS profile for a CRC comprising analysis of the biomarkers in the molecular intelligence (MI) profile and the additional biomarker in FIG. 33N, i.e., 1, 2, 3, 4, 5, 6, 7, 8, 9, 10 or 11 of CDH1, ERBB4, FBXW7, HNF1A, JAK3, NPM1, PTPNI 1, RB1, SMAD4, SMARCB1 and STK11, which may be assessed as indicated this paragraph and/or as in FIG. 33N or Table 15 below. The invention further provides a report comprising results of the molecular profiling and corresponding candidate treatments that are identified as likely beneficial or likely not beneficial, as further described herein.

Table 15 presents a view of the information that is reported for the colorectal cancer molecular intelligence molecular profiles, which can be interpreted as described for Table 7 above. The biomarker-treatment associations for the molecular intelligence molecular profiles for colorectal cancer may comprise those associations in Table 16, which can be interpreted as described for Table 8 above.

TABLE 15
Molecular Profile and Report Parameters: Colorectal Cancer (CRC)

| Agent(s)/Biomarker Status Reported | Biomarker | Platform |
|---|---|---|
| cetuximab, panitumumab | KRAS | NGS |
| | BRAF | NGS |
| | NRAS | NGS |
| | PIK3CA | NGS |
| | PTEN | IHC |
| fluorouracil, capecitabine, pemetrexed | TS | IHC |
| irinotecan | TOPO1 | IHC |
| protein expression status | AR | IHC |
| | ER | IHC |
| | PR | IHC |
| imatinib | cKIT | NGS |
| | PDGFRA | NGS |
| doxorubicin, liposomal-doxorubicin, | HER2 | FISH/CISH |
| epirubicin | TOP2A | IHC |
| | PgP | IHC |
| trastuzumab, lapatinib, pertuzumab, T- | HER2 | IHC, |
| DM1 | | FISH/CISH |
| gemcitabine | RRM1 | IHC |
| temozolomide, dacarbazine | MGMT | IHC |
| docetaxel, paclitaxel, nab-paclitaxel | TLE3 | IHC |
| | TUBB3 | IHC |
| | PgP | IHC |
| | SPARCm | IHC |
| | SPARCp | IHC |
| vandetanib | RET | NGS |
| clinical trials | cMET | IHC, |
| | | FISH/CISH |
| clinical trials | VHL | NGS |
| clinical trials | PTEN | NGS |
| clinical trials | IDH1 | NGS |
| clinical trials | ABL1 | NGS |
| clinical trials | AKT1 | NGS |
| clinical trials | ALK | NGS |
| clinical trials | APC | NGS |
| clinical trials | ATM | NGS |
| clinical trials | CSF1R | NGS |
| clinical trials | CTNNB1 | NGS |
| clinical trials | EGFR | NGS |
| clinical trials | ERBB2 | NGS |
| | (HER2) | |
| clinical trials | FGFR1 | NGS |
| clinical trials | FGFR2 | NGS |
| clinical trials | FLT3 | NGS |
| clinical trials | GNAQ | NGS |
| clinical trials | GNA11 | NGS |
| clinical trials | GNAS | NGS |
| clinical trials | HRAS | NGS |
| clinical trials | JAK2 | NGS |
| clinical trials | KDR | NGS |
| | (VEGFR2) | |
| clinical trials | cMET | NGS |
| clinical trials | MLH1 | NGS |
| clinical trials | MPL | NGS |
| clinical trials | NOTCH1 | NGS |
| clinical trials | SMO | NGS |
| clinical trials | TP53 | NGS |

TABLE 16
Rules for Colorectal Cancer Biomarker-Drug Associations

| | | Biomarker | Biomarker | Biomarker | Biomarker | Biomarker | Overall |
|---|---|---|---|---|---|---|---|
| Drug Class | Drugs | Result | Result | Result | Result | Result | Benefit |
| Monoclonal | cetuximab, | KRAS | BRAF | NRAS | PIK3CA | PTEN | Overall |
| antibodies | panitumumab | Mutated | V600E | Mutated | Mutated 1 | Negative | Benefit |
| (EGFR- | | (Seq.) | (Seq.) | (Seq.) | exon20 | (IHC) | |
| targeted) | | | | | (Seq.) | | |
| | | T | Any | Any | Any | Any | F |
| | | F or G13D | Any | Any | Any | Any | T |
| | | No Data | Any | Any | Any | Any | Indet. |
| Antimetabolites | gemcitabine | RRM1 | | | | | Overall |
| | | Negative | | | | | benefit |
| | | (IHC) | | | | | |
| | | T | | | | | T |
| | | F | | | | | F |
| | | No Data | | | | | Indet. |
| Antimetabolites | fluorouracil, | TS | | | | | Overall |
| | capecitabine, | Negative | | | | | benefit |
| | pemetrexed | (IHC) | | | | | |
| | | T | | | | | T |
| | | F | | | | | F |
| | | No Data | | | | | Indet. |
| Topo1 | irinotecan | TOPO1 | | | | | Overall |
| inhibitors | | Positive | | | | | benefit |
| | | (IHC) | | | | | |
| | | T | | | | | T |
| | | F | | | | | F |
| | | No Data | | | | | Indet. |
| Alkylating | temozolomide, | MGMT | | | | | Overall |
| agents | dacarbazine | Negative | | | | | benefit |
| | | (IHC) | | | | | |
| | | T | | | | | T |
| | | F | | | | | F |
| | | No Data | | | | | Indet. |
| TKI | lapatinib | HER2 | HER2 | | | | Overall |
| | | Positive | Amplified | | | | Benefit |
| | | (IHC) | (ISH) | | | | |
| | | T | Any | | | | T |
| | | F, | T or | | | | T |
| | | Equivocal | Equivocal | | | | |
| | | or No Data | High | | | | |
| | | F or | F or | | | | F |
| | | Equivocal | Equivocal | | | | |
| | | | Low | | | | |
| | | F or | No Data | | | | Indet. |
| | | Equivocal | | | | | |
| | | No Data | F, | | | | Indet. |
| | | | Equivocal | | | | |
| | | | Low or No | | | | |
| | | | Data | | | | |
| Monoclonal | trastuzumab, | HER2 | HER2 | | | | Overall |
| antibodies | pertuzumab, ado- | Positive | Amplified | | | | Benefit |
| (Her2- | trastuzumab | (IHC) | (ISH) | | | | |
| Targeted) | emtansine (T- | | | | | | |
| | DM1) | | | | | | |
| | | T | Any | | | | T |
| | | F, | T or | | | | T |
| | | Equivocal | Equivocal | | | | |
| | | or No Data | High | | | | |
| | | F or | F or | | | | F |
| | | Equivocal | Equivocal | | | | |
| | | | Low | | | | |
| | | F or | No Data | | | | Indet. |
| | | Equivocal | | | | | |
| | | No Data | F, | | | | Indet. |
| | | | Equivocal | | | | |
| | | | Low or No | | | | |
| | | | Data | | | | |
| TKI | crizotinib | ALK | ROS1 | | | | Overall |
| | | Positive | Positive | | | | Benefit |
| | | (ISH) | (ISH) | | | | |
| | | T | Any | | | | T |
| | | F or No | T | | | | T |
| | | Data | | | | | |
| | | F | F or No | | | | F |
| | | | Data | | | | |
| | | No Data | F or No | | | | Indet. |
| | | | Data | | | | |
| Anthracyclines | doxorubicin, | TOP2A | Her2 | TOP2A | PGP | | Overall |
| and related | liposomal- | Amplified | Amplified | Positive | Positive | | Benefit |
| substances | doxorubicin, | (ISH) | (ISH) | (IHC) | (IHC) | | |
| | epirubicin | | | | | | |
| | | T | Any | Any | Any | | T |
| | | F or No | T or | Any | Any | | T |
| | | Data | Equivocal | | | | |
| | | | High | | | | |
| | | F or No | F, | T | Any | | T |
| | | Data | Equivocal | | | | |
| | | | Low or No | | | | |
| | | | Data | | | | |
| | | F | F, | F or No | Any | | F |
| | | | Equivocal | Data | | | |
| | | | Low or No | | | | |
| | | | Data | | | | |
| | | No Data | F, | F | Any | | F |
| | | | Equivocal | | | | |
| | | | Low or No | | | | |
| | | | Data | | | | |
| | | No Data | F or | No Data | Any | | F |
| | | | Equivocal | | | | |
| | | | Low | | | | |
| | | No Data | No Data | No Data | T | | F |
| | | No Data | No Data | No Data | F | | T |
| | | No Data | No Data | No Data | No Data | | Indet. |
| TKI | imatinib | c-KIT | PDGFRA | | | | Overall |
| | | exon11 \| | exon 12 \| | | | | Benefit |
| | | exon13 | exon 14 \| | | | | |
| | | (Seq.) | exon 18 | | | | |
| | | | (Seq.) | | | | |
| | | Any | D842V | | | | F |
| | | V654A | Any | | | | F |
| | | T | Any other | | | | T |
| | | F, exon 14, | T | | | | T |
| | | exon 17, | | | | | |
| | | exon 18 or | | | | | |
| | | No Data | | | | | |
| | | F, exon 14, | F or No | | | | Indet. |
| | | exon 17, | Data | | | | |
| | | exon 18 or | | | | | |
| | | No Data | | | | | |
| TKI (RET- | vandetanib | RET | | | | | Overall |
| targeted) | | Mutated | | | | | benefit |
| | | (Seq.) | | | | | |
| | | T | | | | | T |
| | | F or No | | | | | Indet. |
| | | Data | | | | | |
| Taxanes | paclitaxel, | SPARC | SPARC | TLE3 | TUBB3 | PGP | Overall |
| | docetaxel, nab- | Positive | Positive | Positive | Positive | Positive | BenefiT |
| | paclitaxel | (Mono | (Poly IHC) | (IHC) | (IHC) | (IHC) | |
| | | IHC) | | | | | |
| | paclitaxel, | Any | Any | T | Any | Any | T |
| | docetaxel, nab- | | | | | | |
| | paclitaxel | | | | | | |
| | nab-paclitaxel | T | Any | F or No | T or No | Any | T |
| | | | | Data | Data | | |
| | paclitaxel, | T | Any | F or No | F | Any | T |
| | docetaxel, nab- | | | Data | | | |
| | paclitaxel | | | | | | |
| | nab-paclitaxel | F or No | T | F or No | T or No | Any | T |
| | | Data | | Data | Data | | |
| | paclitaxel, | F or No | T | F or No | F | Any | T |
| | docetaxel, nab- | Data | | Data | | | |
| | paclitaxel | | | | | | |
| | paclitaxel, | F or No | F | F | T or No | Any | F |
| | docetaxel, nab- | Data | | | Data | | |
| | paclitaxel | | | | | | |
| | paclitaxel, | F or No | F | F or No | F | Any | T |
| | docetaxel, nab- | Data | | Data | | | |
| | paclitaxel | | | | | | |
| | paclitaxel, | F or No | F | No Data | T | Any | F |
| | docetaxel, nab- | Data | | | | | |
| | paclitaxel | | | | | | |
| | nab-paclitaxel | F | F or No | No Data | No Data | Any | F |
| | | | Data | | | | |
| | paclitaxel, | F | No Data | F | T or No | Any | F |
| | docetaxel, nab- | | | | Data | | |
| | paclitaxel | | | | | | |
| | paclitaxel, | F or No | No Data | F or No | F | Any | T |
| | docetaxel, nab- | Data | | Data | | | |
| | paclitaxel | | | | | | |
| | paclitaxel, | F or No | No Data | No Data | T | Any | F |
| | docetaxel, nab- | Data | | | | | |
| | paclitaxel | | | | | | |
| | nab-paclitaxel | No Data | F | No Data | No Data | Any | F |
| | paclitaxel, | No Data | No Data | F | T or No | Any | F |
| | docetaxel, nab- | | | | Data | | |
| | paclitaxel | | | | | | |
| | paclitaxel, | No Data | No Data | No Data | No Data | Any | Indet. |
| | docetaxel, nab- | | | | | | |
| | paclitaxel | | | | | | |

In an aspect, the invention provides molecular intelligence (MI) profiles for a lung cancer, including without limitation a non-small cell lung cancer (NSCLC) or bronchioloalveolar cancer (BAC or LBAC), comprising assessment of one or more, e.g., e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58 or 59 of: ABL1, AKT1, ALK, APC, AR, ATM, BRAF, CDH1, cKIT, cMET, CSFIR, CTNNB1, EGFR, ER, ERBB2, ERBB4, FBXW7, FGFR1, FGFR2, FLT3, GNA11, GNAQ, GNAS, HER2, HNF1A, HRAS, IDH1, JAK2, JAK3, KDR (VEGFR2), KRAS, MGMT, MLH1, MPL, NOTCH1, NPM1, NRAS, PDGFRA, PGP, PIK3CA, PR, PTEN, PTPN11, RB1, RET, ROS1, RRM1, SMAD4, SMARCB1, SMO, SPARC, STK11, TLE3, TOP2A, TOPO1, TP53, TS, TUBB3, VHL. In one embodiment, ISH is used to assess one or more, e.g., 1, 2, 3, or 4, of: ALK, cMET, HER2, ROS1. Any useful ISH technique can be used. For example, FISH can be used to assess one or two of: ALK and ROS1; and CISH can be used to assess HER2 and cMET. CISH can also be used to assess ALK and/or ROS1. As desired, FISH can be used to assess HER2 and/or cMET. In an embodiment, protein analysis such as IHC is used to assess one or more, e.g., 1, 2, 3, 4, 5, 6, 7, 8, 9, 10, 11, 12, 13, 14, 15, 16 or 17 of: AR, cMET, EGFR (H-score), ER, HER2, MGMT, PGP, PR, PTEN, RRM1, SPARCm, SPARCp, TLE3, TOP2A, TOPO1, TS, TUBB3. “m” and “p” as in SPARC (m/p) refer to IHC performed with monoclonal (“m”) or polyclonal (“p”) primary antibodies. EGFR can be assessed using an H-score, as described herein. In some embodiments, sequence analysis is used to assess one or more, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44 or 45 of: ABL1, AKT1, ALK, APC, ATM, BRAF, CDH1, cKIT, cMET, CSF1R, CTNNB1, EGFR, ERBB2, ERBB4, FBXW7, FGFR1, FGFR2, FLT3, GNA11, GNAQ, GNAS, HNF1A, HRAS, IDH1, JAK2, JAK3, KDR (VEGFR2), KRAS, MLH1, MPL, NOTCH1, NPM1, NRAS, PDGFRA, PIK3CA, PTEN, PTPN11, RB1, RET, SMAD4, SMARCB1, SMO, STK11, TP53, VHL. For example, the sequence analysis can be performed on one or more, e.g., 1, 2, 3, 4, 5, 6, 7, 8, 9, 10, 11, 12, 13 or 14 of ABL1, APC, BRAF, EGFR, FLT3, GNAQ, IDH1, JAK2, cKIT, KRAS, MPL, NRAS, PDGFRA, VHL. The sequence analysis can also be performed on one or more, e.g., 1, 2, 3, 4, 5, 6, 7, 8, 9, 10, 11, 12, 13, 14 or 15 of ABL1, APC, BRAF, EGFR, FLT3, GNAQ, IDH1, JAK2, cKIT, KRAS, MPL, NPM1, NRAS, PDGFRA, VHL. The sequencing may be performed using Next Generation sequencing technology or other technologies as described herein. The molecular profile can be based on assessing the biomarkers as illustrated in FIGS. 33I-J or Table 17 below.

In an embodiment, the invention provides a molecular intelligence (MI) profile for a lung cancer comprising analysis of the biomarkers in FIG. 33I, which may be assessed as indicated in the paragraph above and/or as in FIG. 33I or Table 17 below. For example, the MI profile for lung cancer may comprise: 1) ISH to assess one or more, e.g., 1, 2, 3 or 4, of: ALK, cMET, HER2, ROS1; 2) IHC to assess one or more, e.g., 1, 2, 3, 4, 5, 6, 7, 8, 9, 10, 11, 12, 13, 14, 15, 16 or 17 of: AR, cMET, EGFR (H-score), ER, HER2, MGMT, PGP, PR, PTEN, RRM1, SPARCm, SPARCp, TLE3, TOP2A, TOPO1, TS, TUBB3; and/or 3) sequence analysis to assess one or more, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33 or 34 of: ABL1, AKT1, ALK, APC, ATM, BRAF, cKIT, cMET, CSF1R, CTNNB1, EGFR, ERBB2, FGFR1, FGFR2, FLT3, GNA11, GNAQ, GNAS, HRAS, IDH1, JAK2, KDR (VEGFR2), KRAS, MLH1, MPL, NOTCH1, NRAS, PDGFRA, PIK3CA, PTEN, RET, SMO, TP53, VHL. In another embodiment, the invention provides a molecular intelligence (MI) PLUS profile for a lung cancer comprising analysis of the biomarkers in the molecular intelligence (MI) profile and the additional biomarker in FIG. 33J, i.e., 1, 2, 3, 4, 5, 6, 7, 8, 9, 10 or 11 of CDH1, ERBB4, FBXW7, HNF1A, JAK3, NPM1, PTPN11, RB1, SMAD4, SMARCB1 and STK11, which may be assessed as indicated this paragraph and/or as in FIG. 33J or Table 17 below. The invention further provides a report comprising results of the molecular profiling and corresponding candidate treatments that are identified as likely beneficial or likely not beneficial, as further described herein.

Table 17 presents a view of the information that is reported for the lung cancer molecular intelligence molecular intelligence molecular profiles, which can be interpreted as described for Table 7 above. The biomarker—treatment associations for the molecular intelligence molecular profiles for lung cancer may comprise those associations in Table 18, which can be interpreted as described for Table 8 above.

TABLE 17
Molecular Profile and Report Parameters:
Lung Cancer, e.g., NSCLC or BAC

| Agent(s)/Biomarker Status Reported | Biomarker | Platform |
|---|---|---|
| erlotinib, gefitinib | EGFR | NGS |
| | KRAS | NGS |
| | cMET | FISH/CISH |
| | PIK3CA | NGS |
| | PTEN | IHC |
| afatinib | EGFR | NGS |
| crizotinib | ALK | FISH |
| | ROS1 | FISH |
| pemetrexed, fluorouracil, capecitabine | TS | IHC |
| gemcitabine | RRM1 | IHC |
| docetaxel, paclitaxel, nab-paclitaxel | TLE3 | IHC |
| | TUBB3 | IHC |
| | PgP | IHC |
| | SPARCm | IHC |
| | SPARCp | IHC |
| cetuximab | EGFR | IHC (H-Score) |
| everolimus, temsirolimus, clinical | PIK3CA | NGS |
| trials | | |
| protein expression status | AR | IHC |
| | ER | IHC |
| | PR | IHC |
| imatinib | cKIT | NGS |
| | PDGFRA | NGS |
| doxorubicin, liposomal-doxorubicin, | HER2 | FISH/CISH |
| epirubicin | TOP2A | IHC |
| | PgP | IHC |
| irinotecan | TOPO1 | IHC |
| temozolomide, dacarbazine | MGMT | IHC |
| vandetanib | RET | NGS |
| clinical trials | cMET | IHC |
| trastuzumab, lapatinib, pertuzumab, | HER2 | IHC, |
| T-DM1, clinical trials | | FISH/CISH |
| clinical trials | BRAF | NGS |
| | KRAS | NGS |
| | NRAS | NGS |
| clinical trials | IDH1 | NGS |
| clinical trials | VHL | NGS |
| clinical trials | PTEN | NGS |
| clinical trials | ABL1 | NGS |
| clinical trials | AKT1 | NGS |
| clinical trials | ALK | NGS |
| clinical trials | APC | NGS |
| clinical trials | ATM | NGS |
| clinical trials | CSF1R | NGS |
| clinical trials | CTNNB1 | NGS |
| clinical trials | EGFR | NGS |
| clinical trials | ERBB2 | NGS |
| | (HER2) | |
| clinical trials | FGFR1 | NGS |
| clinical trials | FGFR2 | NGS |
| clinical trials | FLT3 | NGS |
| clinical trials | GNAQ | NGS |
| clinical trials | GNA11 | NGS |
| clinical trials | GNAS | NGS |
| clinical trials | HRAS | NGS |
| clinical trials | JAK2 | NGS |
| clinical trials | KDR | NGS |
| | (VEGFR2) | |
| clinical trials | cMET | NGS |
| clinical trials | MLH1 | NGS |
| clinical trials | MPL | NGS |
| clinical trials | NOTCH1 | NGS |
| clinical trials | SMO | NGS |
| clinical trials | TP53 | NGS |

TABLE 18
Rules for Lung Cancer Biomarker-Drug Associations

| Class of | | Biomarker | Biomarker | Biomarker | Biomarker | Biomarker | Biomarker | Biomarker | Overall |
|---|---|---|---|---|---|---|---|---|---|
| Drugs | Drugs | Result | Result | Result | Result | Result | Result | Result | Benefit |
| TKI | erlotinib | EGFR | EGFR | KRAS | EGFR | cMET | PIK3CA | PTEN | Overall |
| | gefitinib | Exon 20 | Activating | Mutated \| | T790M | Amplified | Mutated \| | Negative | Benefit |
| | | insert | Mutation \| | G13D | Present | (ISH) | exon20 | (IHC) | |
| | | Present | Exon 21 | (Seq.) | (Seq.) | | (Seq.) | | |
| | | (Seq.) | L858R \| | | | | | | |
| | | | Exon 19 | | | | | | |
| | | | del (Seq.) | | | | | | |
| | | T | T | F or no | Any | Any | Any | Any | T |
| | | | | Data | | | | | |
| | | Any | T | T | Any | Any | Any | Any | Indet. |
| | | Any | F | Any | Any | Any | Any | Any | F |
| | | F or No | D | F or No | Any | Any | Any | Any | T |
| | | Data | | Data | | | | | |
| | | F | No Data | F | Any | Any | Any | Any | Indet. |
| | | F | No Data | T or No | Any | Any | Any | Any | F |
| | | | | Data | | | | | |
| | | No Data | No Data | F or No | Any | Any | Any | Any | Indet. |
| | | | | Data | | | | | |
| | | No Data | No Data | T | Any | Any | Any | Any | F |
| Antimetabolic | gemcitabine | RRM1 | | | | | | | Overall |
| | | Negative | | | | | | | Benefit |
| | | (ICH) | | | | | | | |
| | | T | | | | | | | T |
| | | F | | | | | | | F |
| | | No Data | | | | | | | Indet. |
| Antimetabolic | fluorouracil, | TS | | | | | | | Overall |
| | capecitabine, | Negative | | | | | | | Benefit |
| | pemetrexed | (ICH) | | | | | | | |
| | | T | | | | | | | T |
| | | F | | | | | | | F |
| | | No Data | | | | | | | Indet. |
| mTOR | everolimus | PIK3CA | | | | | | | Overall |
| inhibitors | temsirolimus | exon20 | | | | | | | Benefit |
| | | (Seq.) | | | | | | | |
| | | T | | | | | | | T |
| | | F or No | | | | | | | Indet. |
| | | Data | | | | | | | |
| Monoclonal | cetuximab | EGFR | | | | | | | Overall |
| Antibodies | | Positive | | | | | | | Benefit |
| (EGFR | | (IHC H- | | | | | | | |
| Targeted- | | Score) | | | | | | | |
| cetuximab) | | | | | | | | | |
| | | T | | | | | | | T |
| | | F | | | | | | | F |
| | | No Data | | | | | | | Indet. |
| TKI | crizotinib | ALK | ROS1 | | | | | | Overall |
| | | Positive | Positive | | | | | | Benefit |
| | | (FISH) | (FISH) | | | | | | |
| | | T | Any | | | | | | T |
| | | F or No | T | | | | | | T |
| | | Data | | | | | | | |
| | | F | F or No | | | | | | F |
| | | | Data | | | | | | |
| | | No Data | F or No | | | | | | Indet. |
| | | | Data | | | | | | |
| Topo1 | irinotecan | TOPO1 | | | | | | | Overall |
| inhibitors | | Positive | | | | | | | benefit |
| | | (IHC) | | | | | | | |
| | | T | | | | | | | T |
| | | F | | | | | | | F |
| | | No Data | | | | | | | Indet. |
| Alkylating | temozolomide, | MGMT | | | | | | | Overall |
| Agents | dacarbazine | Negative | | | | | | | benefit |
| | | (IHC) | | | | | | | |
| | | T | | | | | | | T |
| | | F | | | | | | | F |
| | | No Data | | | | | | | Indet. |
| TKI | lapatinib | HER2 | HER2 | | | | | | Overall |
| | | Positive | Amplified | | | | | | Benefit |
| | | (IHC) | (FISH) | | | | | | |
| | | T | Any | | | | | | T |
| | | F, | T or | | | | | | T |
| | | Equivocal | Equivocal | | | | | | |
| | | or No Data | High | | | | | | |
| | | F or | F or | | | | | | F |
| | | Equivocal | Equivocal | | | | | | |
| | | | Low | | | | | | |
| | | F or | No Data | | | | | | Indet. |
| | | Equivocal | | | | | | | |
| | | No Data | F, | | | | | | Indet. |
| | | | Equivocal | | | | | | |
| | | | Low or No | | | | | | |
| | | | Data | | | | | | |
| Monoclonal | trastuzumab, | HER2 | HER2 | | | | | | Overall |
| antibodies | pertuzumab, | Positive | Amplified | | | | | | Benefit |
| (Her2- | ado- | (IHC) | (FISH) | | | | | | |
| Targeted) | trastuzumab | | | | | | | | |
| | emtansine (T- | | | | | | | | |
| | DM1) | | | | | | | | |
| | | T | Any | | | | | | T |
| | | F, | T or | | | | | | T |
| | | Equivocal | Equivocal | | | | | | |
| | | or No Data | High | | | | | | |
| | | F or | F or | | | | | | F |
| | | Equivocal | Equivocal | | | | | | |
| | | | Low | | | | | | |
| | | F or | No Data | | | | | | Indet. |
| | | Equivocal | | | | | | | |
| | | No Data | F, | | | | | | Indet. |
| | | | Equivocal | | | | | | |
| | | | Low or No | | | | | | |
| | | | Data | | | | | | |
| Anthracyclines | doxorubicin, | TOP2A | Her2 | TOP2A | PGP | | | | Overall |
| and related | liposomal- | Amplified | Amplified | Positive | Positive | | | | Benefit |
| substances | doxorubicin, | (FISH) | (FISH) | (IHC) | (IHC) | | | | |
| | epirubicin | | | | | | | | |
| | | T | Any | Any | Any | | | | T |
| | | F or No | T or | Any | Any | | | | T |
| | | Data | Equivocal | | | | | | |
| | | | High | | | | | | |
| | | F or No | F, | T | Any | | | | T |
| | | Data | Equivocal | | | | | | |
| | | | Low or No | | | | | | |
| | | | Data | | | | | | |
| | | F | F, | F or No | Any | | | | F |
| | | | Equivocal | Data | | | | | |
| | | | Low or No | | | | | | |
| | | | Data | | | | | | |
| | | No Data | F, | F | Any | | | | F |
| | | | Equivocal | | | | | | |
| | | | Low or No | | | | | | |
| | | | Data | | | | | | |
| | | No Data | F or | No Data | Any | | | | F |
| | | | Equivocal | | | | | | |
| | | | Low | | | | | | |
| | | No Data | No Data | No Data | T | | | | F |
| | | No Data | No Data | No Data | F | | | | T |
| | | No Data | No Data | No Data | No Data | | | | Indet. |
| TKI | imatinib | c-KIT | PDGFRA | | | | | | Overall |
| | | exon11 \| | exon 12 \| | | | | | | Benefit |
| | | exon13 | exon 14 \| | | | | | | |
| | | (Seq.) | exon 18 | | | | | | |
| | | | (Seq.) | | | | | | |
| | | Any | D842V | | | | | | F |
| | | V654A | Any | | | | | | F |
| | | T | Any other | | | | | | T |
| | | F, exon 14, | T | | | | | | T |
| | | exon 17, | | | | | | | |
| | | exon 18 or | | | | | | | |
| | | No Data | | | | | | | |
| | | F, exon 14, | F or No | | | | | | Indet. |
| | | exon 17, | Data | | | | | | |
| | | exon 18 or | | | | | | | |
| | | No Data | | | | | | | |
| TKI (RET- | vandetanib | RET | | | | | | | Overall |
| targeted) | | mutated | | | | | | | benefit |
| | | (Seq.) | | | | | | | |
| | | T | | | | | | | T |
| | | F or No | | | | | | | Indet. |
| | | Data | | | | | | | |
| Taxanes | paclitaxel, | SPARC | SPARC | TLE3 | TUBB3 | PGP | | | Overall |
| | docetaxel, | Positive | Positive | Positive | Positive | Positive | | | Benefit |
| | nab-paclitaxel | (Mono | (Poly | (IHC) | (IHC) | (IHC) | | | |
| | | IHC) | IHC) | | | | | | |
| | paclitaxel, | Any | Any | T | Any | Any | | | T |
| | docetaxel, nab- | | | | | | | | |
| | paclitaxel | | | | | | | | |
| | nab-paclitaxel | T | Any | F or No | T or No | Any | | | T |
| | | | | Data | Data | | | | |
| | paclitaxel, | T | Any | F or No | F | Any | | | T |
| | docetaxel, nab- | | | Data | | | | | |
| | paclitaxel | | | | | | | | |
| | nab-paclitaxel | F or No | T | F or No | T or No | Any | | | T |
| | | Data | | Data | Data | | | | |
| | paclitaxel, | F or No | T | F or No | F | Any | | | T |
| | docetaxel, nab- | Data | | Data | | | | | |
| | paclitaxel | | | | | | | | |
| | paclitaxel, | F or No | F | F | T or No | Any | | | F |
| | docetaxel, nab- | Data | | | Data | | | | |
| | paclitaxel | | | | | | | | |
| | paclitaxel, | F or No | F | F or No | F | Any | | | T |
| | docetaxel, nab- | Data | | Data | | | | | |
| | paclitaxel | | | | | | | | |
| | paclitaxel, | F or No | F | No Data | T | Any | | | F |
| | docetaxel, nab- | Data | | | | | | | |
| | paclitaxel | | | | | | | | |
| | nab-paclitaxel | F | F or No | No Data | No Data | Any | | | F |
| | | | Data | | | | | | |
| | paclitaxel, | F | No Data | F | T or No | Any | | | F |
| | docetaxel, nab- | | | | Data | | | | |
| | paclitaxel | | | | | | | | |
| | paclitaxel, | F or No | No Data | F or No | F | Any | | | T |
| | docetaxel, nab- | Data | | Data | | | | | |
| | paclitaxel | | | | | | | | |
| | paclitaxel, | F or No | No Data | No Data | T | Any | | | F |
| | docetaxel, nab- | Data | | | | | | | |
| | paclitaxel | | | | | | | | |
| | nab-paclitaxel | No Data | F | No Data | No Data | Any | | | F |
| | paclitaxel, | No Data | No Data | F | T or No | Any | | | F |
| | docetaxel, nab- | | | | Data | | | | |
| | paclitaxel | | | | | | | | |
| | paclitaxel, | No Data | No Data | No Data | No Data | Any | | | Indet. |
| | docetaxel, nab- | | | | | | | | |
| | paclitaxel | | | | | | | | |
| TKI (EGFR- | afatinib | EGFR | EGFR | EGFR | | | | | Overall |
| targeted) | | activating | T790M | Exon 20 | | | | | benefit |
| | | mutation | Present | insert | | | | | |
| | | (Seq.) | (Seq.) | Present | | | | | |
| | | | | (Seq.) | | | | | |
| | | T, F, | Any | Any | | | | | Indet. |
| | | exon20ins | | | | | | | |
| | | or No Data | | | | | | | |
| | | Exon 21 | Any | Any | | | | | T |
| | | L858R or | | | | | | | |
| | | Exon 19 | | | | | | | |
| | | del | | | | | | | |
| | | F | F or No | F or No | | | | | F |
| | | | Data | Data | | | | | |

When assessing lung cancer, the T790M mutation in EGFR may further implicate treatment decisions as follows. First, the following information can be reported when EGFR T790M is detected concomitantly with an exon19 deletion or L858R EGFR mutation: The presence of T790M mutation in EGFR has been associated with higher likelihood of prolonged efficacy (PFS/OS) with afatinib than gefitinib or erlotinib. See, e.g., Metro, G., L. Crino, (2011) “The LUX-Lung clinical trial program of afatinib for non-small-cell lung cancer.” Expert Rev Anticancer Ther. 11(5):673-82; which reference is incorporated herein in its entirety. Recent data including AMP, CAP and NCCN guidelines support the continued use of EGFR TKIs in lung adenocarcinoma patients with EGFR activating mutations after the acquisition of a secondary mutation in EGFR-T790M that renders the kinase resistant to erlotinib or gefitinib. To overcome resistance, EGFR remains a drug target and discontinuation of EGFR TKIs may lead to further progression of the disease. See, e.g., Lindeman, N.I., M. Ladanyi, et al. (2013) “Molecular testing guideline for selection of lung cancer patients for EGFR and ALK tyrosine kinase inhibitors: guideline from the College of American Pathologists, International Association for the Study of Lung Cancer, and Association for Molecular Pathology.” Arch Pathol Lab Med, 137(6):828-60; which reference is incorporated herein in its entirety. Second, the following information can be reported when T790M is detected concomitantly with an activating EGFR mutation other than an exon 19 deletion or L858R: Recent data including AMP, CAP and NCCN guidelines support the continued use of EGFR TKIs in lung adenocarcinoma patients with EGFR activating mutations after the acquisition of a secondary mutation in EGFR-T790M that renders the kinase resistant to erlotinib or gefitinib. To overcome resistance, EGFR remains a drug target and discontinuation of EGFR TKIs may lead to further progression of the disease. See e.g., Lindeman, et al. 2013.

In an aspect, the invention provides molecular intelligence (MI) profiles for a glioma comprising assessment of one or more, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 or 61, of: ABL1, AKT1, ALK, APC, AR, ATM, BRAF, CDH1, cKIT, cMET, CSF1R, CTNNB1, EGFR, EGFRvIII, ER, ERBB2, ERBB4, FBXW7, FGFR1, FGFR2, FLT3, GNA11, GNAQ, GNAS, HER2, HNF1A, HRAS, IDH1, IDH2, JAK2, JAK3, KDR (VEGFR2), KRAS, MGMT-Me, MLH1, MPL, NOTCH1, NPM1, NRAS, PDGFRA, PGP, PIK3CA, PR, PTEN, PTPN11, RB1, RET, RRM1, SMAD4, SMARCB1, SMO, SPARCm, SPARCp, STK11, TLE3, TOP2A, TOPO1, TP53, TS, TUBB3, VHL. The invention further provides a method of selecting a candidate treatment for a glioma comprising assessment of one or more members of the glioma molecular profile using one or more molecular profiling technique presented herein, e.g., ISH (e.g., FISH, CISH), IHC, RT-PCR, expression array, mutation analysis (e.g., NextGen sequencing, Sanger sequencing, pyrosequencing, Fragment analysis (FA, e.g., RFLP), PCR), etc. In one embodiment, ISH is used to assess one or more, e.g., 1 or 2, of: cMET, HER2. Any useful ISH technique can be used. For example, FISH can be used to assess cMET and/or HER2; or CISH can be used to assess cMET and/or HER2. In an embodiment, protein analysis such as IHC is used to assess one or more, e.g., 1, 2, 3, 4, 5, 6, 7, 8, 9, 10, 11, 12, 13, 14 or 15, of: AR, cMET, ER, HER2, PGP, PR, PTEN, RRM1, SPARCm, SPARCp, TLE3, TOP2A, TOPO1, TS, TUBB3. “m” and “p” as in SPARC (m/p) refer to IHC performed with monoclonal (“m”) or polyclonal (“p”) primary antibodies. In some embodiments, sequence analysis is used to assess one or more, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44 or 45 of: ABL1, AKT1, ALK, APC, ATM, BRAF, CDH1, cKIT, cMET, CSF1R, CTNNB1, EGFR, ERBB2, ERBB4, FBXW7, FGFR1, FGFR2, FLT3, GNA11, GNAQ, GNAS, HNF1A, HRAS, IDH1, JAK2, JAK3, KDR (VEGFR2), KRAS, MLH1, MPL, NOTCH1, NPM1, NRAS, PDGFRA, PIK3CA, PTEN, PTPN11, RB1, RET, SMAD4, SMARCB1, SMO, STK11, TP53, VHL. For example, the sequence analysis can be performed on one or more, e.g., 1, 2, 3, 4, 5, 6, 7, 8, 9, 10, 11, 12, 13 or 14 of ABL1, APC, BRAF, EGFR, FLT3, GNAQ, IDH1, JAK2, cKIT, KRAS, MPL, NRAS, PDGFRA, VHL. The sequence analysis can also be performed on one or more, e.g., 1, 2, 3, 4, 5, 6, 7, 8, 9, 10, 11, 12, 13, 14 or 15 of ABL1, APC, BRAF, EGFR, FLT3, GNAQ, IDH1, JAK2, cKIT, KRAS, MPL, NPM1, NRAS, PDGFRA, VHL. The sequencing may be performed using Next Generation sequencing technology or other technologies as described herein. Sequence analysis can also be performed for one or more of MGMT, IDH2 and EGFRvIII. For example, methylation of the MGMT promoter region can be assessed using pyrosequencing, mutation of IDH2 can be assess by Sanger sequencing, and/or the presence of EGFRvIII can be detected using fragment analysis. The molecular profile can be based on assessing the biomarkers as illustrated in FIGS. 33O-P or Table 21 below.

In an embodiment, the invention provides a molecular intelligence (MI) profile for a glioma comprising analysis of the biomarkers in FIG. 33O, which may be assessed as indicated in the paragraph above and/or as in FIG. 33O or Table 21 below. For example, the MI profile for a glioma may comprise: 1) ISH to assess one or more, e.g., 1 or 2, of: cMET, HER2; 2) IHC to assess one or more, e.g., 1, 2, 3, 4, 5, 6, 7, 8, 9, 10, 11, 12, 13, 14 or 15, of: AR, cMET, ER, HER2, PGP, PR, PTEN, RRM1, SPARCm, SPARCp, TLE3, TOP2A, TOPO1, TS, TUBB3; 3) sequence analysis to assess one or more, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33 or 34 of: ABL1, AKT1, ALK, APC, ATM, BRAF, cKIT, cMET, CSF1R, CTNNB1, EGFR, ERBB2, FGFR1, FGFR2, FLT3, GNA11, GNAQ, GNAS, HRAS, IDH1, JAK2, KDR (VEGFR2), KRAS, MLH1, MPL, NOTCH1, NRAS, PDGFRA, PIK3CA, PTEN, RET, SMO, TP53, VHL; 4) sequence analysis, e.g., pyrosequencing, to assess promoter methylation of MGMT; 5) sequence analysis, e.g., Sanger sequencing, or IDH2; and/or 6) detection of the EGFRvIII variant, e.g., as assessed by fragment analysis. In another embodiment, the invention provides a molecular intelligence (MI) PLUS profile for a glioma comprising analysis of the biomarkers in the molecular intelligence (MI) profile and the additional biomarker in FIG. 33P, i.e., 1, 2, 3, 4, 5, 6, 7, 8, 9, 10 or 11 of CDH1, ERBB4, FBXW7, HNF1A, JAK3, NPM1, PTPN11, RB1, SMAD4, SMARCB1 and STK11, which may be assessed as indicated this paragraph and/or as in FIG. 33P or Table 21 below. The invention further provides a report comprising results of the molecular profiling and corresponding candidate treatments that are identified as likely beneficial or likely not beneficial, as further described herein.

Table 21 below presents a view of the information that is reported for the glioblastoma molecular intelligence molecular profile, which can be interpreted as described for Table 7 above. The biomarker-treatment associations for the molecular profile for glioblastoma may comprise those associations in Table 19, which can be interpreted as described for Table 8 above.

TABLE 19
Rules for Glioma Biomarker-Drug Associations

| | | Biomarker | Biomarker | Biomarker | Biomarker | Biomarker | Overall |
|---|---|---|---|---|---|---|---|
| Class of Drugs | Drugs | Result | Result | Result | Result | Result | benefit |
| Antimetabolites | gemcitabine | RRM1 | | | | | Overall |
| (gemcitabine) | | Negative | | | | | benefit |
| | | (IHC) | | | | | |
| | | T | | | | | T |
| | | F | | | | | F |
| | | No Data | | | | | Indet. |
| Antimetabolites | fluorouracil, | TS | | | | | Overall |
| | capecitabine, | Negative | | | | | benefit |
| | pemetrexed | (IHC) | | | | | |
| | | T | | | | | T |
| | | F | | | | | F |
| | | No Data | | | | | Indet. |
| Topo1 | irinotecan, | TOPO1 | | | | | Overall |
| inhibitors | topotecan | Positive | | | | | benefit |
| | | (IHC) | | | | | |
| | | T | | | | | T |
| | | F | | | | | F |
| | | No Data | | | | | Indet. |
| Alkylating | temozolomide, | MGMT | MGMT | | | | Overall |
| agents | dacarbazine | Negative | Methylated | | | | benefit |
| | | (IHC) | (Pyro.) | | | | |
| | | Any | T | | | | T |
| | | Any | F | | | | F |
| | | T | Equivocal or | | | | T |
| | | | No Data | | | | |
| | | F | Equivocal or | | | | F |
| | | | No Data | | | | |
| | | No Data | Equivocal or | | | | Indet. |
| | | | No Data | | | | |
| mTOR | everolimus, | PIK3CA | | | | | Overall |
| inhibitors | temsirolimus | exon20 | | | | | Benefit |
| | | (Seq.) | | | | | |
| | | T | | | | | T |
| | | F or No | | | | | Indet. |
| | | Data | | | | | |
| Anti-androgens | bicalutamide, | AR Positive | | | | | Overall |
| | flutamide, | (IHC) | | | | | Benefit |
| | abiraterone | | | | | | |
| | | T | | | | | T |
| | | F | | | | | F |
| | | No Data | | | | | Indet. |
| Hormonal | tamoxifen, | ER Positive | PR Positive | | | | Overall |
| Agents | toremifene, | (IHC) | (MC) | | | | Benefit |
| | fulvestrant, | | | | | | |
| | letrozole, | | | | | | |
| | anastrozole, | | | | | | |
| | exemestane, | | | | | | |
| | megestrol | | | | | | |
| | acetate, | | | | | | |
| | leuprolide, | | | | | | |
| | goserelin | | | | | | |
| | | T | Any | | | | T |
| | | F or No | T | | | | T |
| | | Data | | | | | |
| | | F | F | | | | F |
| | | F | No Data | | | | Indet. |
| | | No Data | F or No | | | | Indet. |
| | | | Data | | | | |
| TKI (lapatinib) | lapatinib | HER2 | HER2 | | | | Overall |
| | | Positive | Amplified | | | | Benefit |
| | | (IHC) | (ISH) | | | | |
| | | T | Any | | | | T |
| | | F, | T or | | | | T |
| | | Equivocal | Equivocal | | | | |
| | | or No Data | High | | | | |
| | | F or | F or | | | | F |
| | | Equivocal | Equivocal | | | | |
| | | | Low | | | | |
| | | F or | No Data | | | | Indet. |
| | | Equivocal | | | | | |
| | | No Data | F, Equivocal | | | | Indet. |
| | | | Low or No | | | | |
| | | | Data | | | | |
| Monoclonal | trastuzumab, | HER2 | HER2 | | | | Overall |
| antibodies | pertuzumab, ado- | Positive | Amplified | | | | Benefit |
| (Her2- | trastuzumab | (IHC) | (ISH) | | | | |
| Targeted) | emtansine (T- | | | | | | |
| | DM1) | | | | | | |
| | | T | Any | | | | T |
| | | F, | T or | | | | T |
| | | Equivocal | Equivocal | | | | |
| | | or No Data | High | | | | |
| | | F or | F or | | | | F |
| | | Equivocal | Equivocal | | | | |
| | | | Low | | | | |
| | | F or | No Data | | | | Indet. |
| | | Equivocal | | | | | |
| | | No Data | F, Equivocal | | | | Indet. |
| | | | Low or No | | | | |
| | | | Data | | | | |
| Anthracyclines | doxorubicin, | TOP2A | Her2 | TOP2A | PGP | | Overall |
| and related | liposomal- | Amplified | Amplified | Positive | Positive | | Benefit |
| substances | doxorubicin, | (ISH) | (ISH) | (IHC) | (IHC) | | |
| | epirubicin | | | | | | |
| | | T | Any | Any | Any | | T |
| | | F or No | T or | Any | Any | | T |
| | | Data | Equivocal | | | | |
| | | | High | | | | |
| | | F or No | F, Equivocal | T | Any | | T |
| | | Data | Low or No | | | | |
| | | | Data | | | | |
| | | F | F, Equivocal | F or No | Any | | F |
| | | | Low or No | Data | | | |
| | | | Data | | | | |
| | | No Data | F, Equivocal | F | Any | | F |
| | | | Low or No | | | | |
| | | | Data | | | | |
| | | No Data | F or | No Data | Any | | F |
| | | | Equivocal | | | | |
| | | | Low | | | | |
| | | No Data | No Data | No Data | T | | F |
| | | No Data | No Data | No Data | F | | T |
| | | No Data | No Data | No Data | No Data | | Indet. |
| TKI | imatinib | c-KIT | PDGFRA | | | | Overall |
| | | exon11 \| | exon 12 \| | | | | Benefit |
| | | exon13 | exon 14 \| | | | | |
| | | (Seq.) | exon 18 | | | | |
| | | | (Seq.) | | | | |
| | | Any | D842V | | | | F |
| | | V654A | Any | | | | F |
| | | T | Any other | | | | T |
| | | F, exon 14, | T | | | | T |
| | | exon 17, | | | | | |
| | | exon 18 or | | | | | |
| | | No Data | | | | | |
| | | F, exon 14, | F or No | | | | Indet. |
| | | exon 17, | Data | | | | |
| | | exon 18 or | | | | | |
| | | No Data | | | | | |
| TKI | crizotinib | ALK | ROS1 | | | | Overall |
| | | Positive | Positive | | | | Benefit |
| | | (ISH) | (ISH) | | | | |
| | | T | Any | | | | T |
| | | F or No | T | | | | T |
| | | Data | | | | | |
| | | F | F or No | | | | F |
| | | | Data | | | | |
| | | No Data | F or No | | | | Indet. |
| | | | Data | | | | |
| TKI (RET- | vandetanib | RET | | | | | Overall |
| targeted) | | mutated | | | | | benefit |
| | | (Seq.) | | | | | |
| | | T | | | | | T |
| | | F or No | | | | | Indet. |
| | | Data | | | | | |
| Taxanes | paclitaxel, | SPARC | SPARC | TLE3 | TUBB3 | PGP | Overall |
| | docetaxel, nab- | (Mono | (Poly IHC) | Positive | Positive | Positive | Benefit |
| | paclitaxel | IHC) | | (IHC) | (IHC) | (IHC) | |
| | paclitaxel, | Any | Any | T | Any | Any | T |
| | docetaxel, nab- | | | | | | |
| | paclitaxel | | | | | | |
| | nab-paclitaxel | T | Any | F or No | T or No | Any | T |
| | | | | Data | Data | | |
| | paclitaxel, | T | Any | F or No | F | Any | T |
| | docetaxel, nab- | | | Data | | | |
| | paclitaxel | | | | | | |
| | nab-paclitaxel | F or No | T | F or No | T or No | Any | T |
| | | Data | | Data | Data | | |
| | paclitaxel, | F or No | T | F or No | F | Any | T |
| | docetaxel, nab- | Data | | Data | | | |
| | paclitaxel | | | | | | |
| | paclitaxel, | F or No | F | F | T or No | Any | F |
| | docetaxel, nab- | Data | | | Data | | |
| | paclitaxel | | | | | | |
| | paclitaxel, | F or No | F | F or No | F | Any | T |
| | docetaxel, nab- | Data | | Data | | | |
| | paclitaxel | | | | | | |
| | paclitaxel, | F or No | F | No Data | T | Any | F |
| | docetaxel, nab- | Data | | | | | |
| | paclitaxel | | | | | | |
| | nab-paclitaxel | F | F or No | No Data | No Data | Any | F |
| | | | Data | | | | |
| | paclitaxel, | F | No Data | F | T or No | Any | F |
| | docetaxel, nab- | | | | Data | | |
| | paclitaxel | | | | | | |
| | paclitaxel, | F or No | No Data | F or No | F | Any | T |
| | docetaxel, nab- | Data | | Data | | | |
| | paclitaxel | | | | | | |
| | paclitaxel, | F or No | No Data | No Data | T | Any | F |
| | docetaxel, nab- | Data | | | | | |
| | paclitaxel | | | | | | |
| | nab-paclitaxel | No Data | F | No Data | No Data | Any | F |
| | paclitaxel, | No Data | No Data | F | T or No | Any | F |
| | docetaxel, nab- | | | | Data | | |
| | paclitaxel | | | | | | |
| | paclitaxel, | No Data | No Data | No Data | No Data | Any | Indet. |
| | docetaxel, nab- | | | | | | |
| | paclitaxel | | | | | | |

In an aspect, the invention provides molecular intelligence (MI) profiles for a gastrointestinal stromal tumor (GIST) comprising assessment of one or more, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57 or 58, of: ABL1, AKT1, ALK, APC, AR, ATM, BRAF, CDH1, cKIT, cMET, CSF1R, CTNNB1, EGFR, ER, ERBB2, ERBB4, FBXW7, FGFR1, FGFR2, FLT3, GNA11, GNAQ, GNAS, HER2, HNF1A, HRAS, IDH1, JAK2, JAK3, KDR (VEGFR2), KRAS, MGMT, MLH1, MPL, NOTCH1, NPM1, NRAS, PDGFRA, PGP, PIK3CA, PR, PTEN, PTPN11, RB1, RET, RRM1, SMAD4, SMARCB1, SMO, SPARC, STK11, TLE3, TOP2A, TOPO1, TP53, TS, TUBB3, VHL. The invention further provides a method of selecting a candidate treatment for GIST comprising assessment of one or more members of the GIST cancer molecular profile using one or more molecular profiling technique presented herein, e.g., ISH (e.g., FISH, CISH), IHC, RT-PCR, expression array, mutation analysis (e.g., NextGen sequencing, Sanger sequencing, pyrosequencing, Fragment analysis (FA, e.g., RFLP), PCR), etc. In one embodiment, ISH is used to assess one or more, e.g., 1 or 2, of: cMET, HER2. Any useful ISH technique can be used. For example, FISH can be used to assess cMET and/or HER2; or CISH can be used to assess cMET and/or HER2. In an embodiment, protein analysis such as IHC is used to assess one or more, e.g., 1, 2, 3, 4, 5, 6, 7, 8, 9, 10, 11, 12, 13, 14, 15 or 16 of: AR, cMET, ER, HER2, MGMT, PGP, PR, PTEN, RRM1, SPARCm, SPARCp, TLE3, TOP2A, TOPO1, TS, TUBB3. “m” and “p” as in SPARC (m/p) refer to IHC performed with monoclonal (“m”) or polyclonal (“p”) primary antibodies. In some embodiments, sequence analysis is used to assess one or more, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44 or 45 of: ABL1, AKT1, ALK, APC, ATM, BRAF, CDH1, cKIT, cMET, CSF1R, CTNNB1, EGFR, ERBB2, ERBB4, FBXW7, FGFR1, FGFR2, FLT3, GNA11, GNAQ, GNAS, HNF1A, HRAS, IDH1, JAK2, JAK3, KDR (VEGFR2), KRAS, MLH1, MPL, NOTCH1, NPM1, NRAS, PDGFRA, PIK3CA, PTEN, PTPN11, RB1, RET, SMAD4, SMARCB1, SMO, STK11, TP53, VHL. For example, the sequence analysis can be performed on one or more, e.g., 1, 2, 3, 4, 5, 6, 7, 8, 9, 10, 11, 12, 13 or 14 of ABL1, APC, BRAF, EGFR, FLT3, GNAQ, IDH1, JAK2, cKIT, KRAS, MPL, NRAS, PDGFRA, VHL. The sequence analysis can also be performed on one or more, e.g., 1, 2, 3, 4, 5, 6, 7, 8, 9, 10, 11, 12, 13, 14 or 15 of ABL1, APC, BRAF, EGFR, FLT3, GNAQ, IDH1, JAK2, cKIT, KRAS, MPL, NPM1, NRAS, PDGFRA, VHL. The sequencing may be performed using Next Generation sequencing technology or other technologies as described herein. The molecular profile can be based on assessing the biomarkers as illustrated in Table 21 below, which table presents a molecular profile for any cancer, including without limitation a solid tumor.

In an embodiment, the invention provides a molecular intelligence (MI) profile for a GIST comprising analysis of the biomarkers in the molecular profile for a GIST, which may be assessed as indicated in the paragraph above and/or as in Table 21 below. For example, the MI profile for GIST may comprise: 1) ISH to assess one or more, e.g., 1 or 2, of: cMET, HER2; 2) IHC to assess one or more, e.g., 1, 2, 3, 4, 5, 6, 7, 8, 9, 10, 11, 12, 13, 14, 15 or 16 of: AR, cMET, ER, HER2, MGMT, PGP, PR, PTEN, RRM1, SPARCm, SPARCp, TLE3, TOP2A, TOPO1, TS, TUBB3; and/or 3) sequence analysis to assess one or more, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33 or 34 of: ABL1, AKT1, ALK, APC, ATM, BRAF, cKIT, cMET, CSF1R, CTNNB1, EGFR, ERBB2, FGFR1, FGFR2, FLT3, GNA11, GNAQ, GNAS, HRAS, IDH1, JAK2, KDR (VEGFR2), KRAS, MLH1, MPL, NOTCH1, NRAS, PDGFRA, PIK3CA, PTEN, RET, SMO, TP53, VHL. In another embodiment, the invention provides a molecular intelligence (MI) PLUS profile for GIST comprising analysis of the biomarkers in the molecular intelligence (MI) profile 1, 2, 3, 4, 5, 6, 7, 8, 9, 10 or 11 of CDH1, ERBB4, FBXW7, HNF1A, JAK3, NPM1, PTPN11, RB1, SMAD4, SMARCB1 and STK11, which may be assessed as indicated this paragraph and/or as in Table 21 below. The invention further provides a report comprising results of the molecular profiling and corresponding candidate treatments that are identified as likely beneficial or likely not beneficial, as further described herein.

Table 21 below presents a view of the information that is reported for GIST molecular intelligence molecular profile, which can be interpreted as described for Table 7 above. The biomarker-treatment associations for the molecular profile for GIST may comprise those associations in Table 20, which can be interpreted as described for Table 8 above.

TABLE 20
Rules for GIST Biomarker-Drug Associations

| | | Biomarker | Biomarker | Biomarker | Biomarker | Biomarker | Overall |
|---|---|---|---|---|---|---|---|
| Class of Drugs | Drugs | Result | Result | Result | Result | Result | benefit |
| | | RRM1 | | | | | |
| | | Negative | | | | | Overall |
| Antimetabolites | gemcitabine | (IHC) | | | | | benefit |
| | | T | | | | | T |
| | | F | | | | | F |
| | | No Data | | | | | Indeterminate |
| | fluorouracil, | TS | | | | | |
| | capecitabine, | Negative | | | | | Overall |
| Antimetabolites | pemetrexed | (IHC) | | | | | benefit |
| | | T | | | | | T |
| | | F | | | | | F |
| | | No Data | | | | | Indeterminate |
| | | TOPO1 | | | | | |
| Topo1 | irinotecan, | Positive | | | | | Overall |
| inhibitors | topotecan | (IHC) | | | | | benefit |
| | | T | | | | | T |
| | | F | | | | | F |
| | | No Data | | | | | Indeterminate |
| | | MGMT | | | | | |
| Alkylating | temozolomide, | Negative | | | | | Overall |
| agents | dacarbazine | (IHC) | | | | | benefit |
| | | T | | | | | T |
| | | F | | | | | F |
| | | No Data | | | | | Indeterminate |
| | | PIK3CA | | | | | |
| mTOR | everolimus, | exon20 | | | | | Overall |
| inhibitors | temsirolimus | (Seq.) | | | | | Benefit |
| | | T | | | | | T |
| | | F or No | | | | | indeterminate |
| | | Data | | | | | |
| | bicalutamide, | AR | | | | | |
| | flutamide, | Positive | | | | | Overall |
| Anti-androgens | abiraterone | (IHC) | | | | | Benefit |
| | | T | | | | | T |
| | | F | | | | | F |
| | | No Data | | | | | Indeterminate |
| | tamoxifen, | | | | | | |
| | toremifene, | | | | | | |
| | fulvestrant, | | | | | | |
| | letrozole, | | | | | | |
| | anastrozole, | | | | | | |
| | exemestane, | | | | | | |
| | megestrol | | | | | | |
| | acetate, | ER | PR | | | | |
| Hormonal | leuprolide, | Positive | Positive | | | | Overall |
| Agents | goserelin | (IHC) | (IHC) | | | | Benefit |
| | | T | Any | | | | T |
| | | F or No | T | | | | T |
| | | Data | | | | | |
| | | F | F | | | | F |
| | | F | No Data | | | | Indet. |
| | | No Data | F or No | | | | Indet. |
| | | | Data | | | | |
| | | HER2 | | | | | |
| | | Positive | HER2 | | | | Overall |
| TKI | lapatinib | (IHC) | Amplified | | | | Benefit |
| | | T | Any | | | | T |
| | | F, | T or | | | | T |
| | | Equivocal | Equivocal | | | | |
| | | or No Data | High | | | | |
| | | F or | F or | | | | F |
| | | Equivocal | Equivocal | | | | |
| | | | Low | | | | |
| | | F or | No Data | | | | Indet. |
| | | Equivocal | | | | | |
| | | No Data | F, | | | | Indet. |
| | | | Equivocal | | | | |
| | | | Low or No | | | | |
| | | | Data | | | | |
| | trastuzumab, | | | | | | |
| | pertuzumab, | | | | | | |
| Monoclonal | ado- | | | | | | |
| antibodies | trastuzumab | HER2 | HER2 | | | | |
| (Her2- | emtansine (T- | Positive | Amplified | | | | Overall |
| Targeted) | DM1) | (IHC) | (ISH) | | | | Benefit |
| | | T | Any | | | | T |
| | | F, | T or | | | | T |
| | | Equivocal | Equivocal | | | | |
| | | or No Data | High | | | | |
| | | F or | F or | | | | F |
| | | Equivocal | Equivocal | | | | |
| | | | Low | | | | |
| | | F or | No Data | | | | Indet. |
| | | Equivocal | | | | | |
| | | No Data | F, | | | | Indet. |
| | | | Equivocal | | | | |
| | | | Low or No | | | | |
| | | | Data | | | | |
| | | c-KIT | | | | | |
| | | exon9 \| | | | | | |
| | | V654A \| | | | | | |
| | | exon 14 | | | | | Overall |
| TKI | sunitinib | (Seq.) | | | | | Benefit |
| | | T or F | | | | | T |
| | | Exon 11, | | | | | F |
| | | Exon 13, | | | | | |
| | | Exon 17 or | | | | | |
| | | Exon 18 | | | | | |
| | | No Data | | | | | Indeterminate |
| | | c-KIT | PDGFRA | | | | |
| | | exon9 \| | exon 12 \| | | | | |
| | | exon11 \| | exon 14 \| | | | | |
| | | exon13 | exon 18 | | | | Overall |
| TKI | imatinib | (Seq.) | (Seq.) | | | | Benefit |
| | | Any | D842V | | | | F |
| | | V654A | Any | | | | F |
| | | T | Any other | | | | T |
| | | F, exon 14, | T | | | | T |
| | | exon 17, | | | | | |
| | | exon 18 or | | | | | |
| | | No Data | | | | | |
| | | F, exon 14, | F or No | | | | Indet. |
| | | exon 17, | Data | | | | |
| | | exon 18 or | | | | | |
| | | No Data | | | | | |
| | | ALK | ROS1 | | | | |
| | | Positive | Positive | | | | Overall |
| TKI | crizotinib | (ISH) | (ISH) | | | | Benefit |
| | | T | Any | | | | T |
| | | F or No | T | | | | T |
| | | Data | | | | | |
| | | F | F or No | | | | F |
| | | | Data | | | | |
| | | No Data | F or No | | | | Indet. |
| | | | Data | | | | |
| | doxorubicin, | | | | | | |
| Anthracyclines | liposomal- | TOP2A | Her2 | TOP2A | PGP | | |
| and related | doxorubicin, | Amplified | Amplified | Positive | Positive | | Overall |
| substances | epirubicin | (ISH) | (ISH) | (IHC) | (IHC) | | Benefit |
| | | T | Any | Any | Any | | T |
| | | F or No | T or | Any | Any | | T |
| | | Data | Equivocal | | | | |
| | | | High | | | | |
| | | F or No | F, | T | Any | | T |
| | | Data | Equivocal | | | | |
| | | | Low or No | | | | |
| | | | Data | | | | |
| | | F | F, | F or No | Any | | F |
| | | | Equivocal | Data | | | |
| | | | Low or No | | | | |
| | | | Data | | | | |
| | | No Data | F, | F | Any | | F |
| | | | Equivocal | | | | |
| | | | Low or No | | | | |
| | | | Data | | | | |
| | | No Data | F or | No Data | Any | | F |
| | | | Equivocal | | | | |
| | | | Low | | | | |
| | | No Data | No Data | No Data | T | | F |
| | | No Data | No Data | No Data | F | | T |
| | | No Data | No Data | No Data | No Data | | Indet. |
| | | RET | | | | | |
| TKI (RET- | | Mutated | | | | | Overall |
| targeted) | vandetanib | (Seq.) | | | | | benefit |
| | | T | | | | | T |
| | | F or No | | | | | Indeterminate |
| | | Data | | | | | |
| | | SPARC | | | | | |
| | paclitaxel, | Positive | SPARC | TLE3 | TUBB3 | PGP | |
| | docetaxel, nab- | (Mono | Positive | Positive | Positive | Positive | Overall |
| Taxanes | paclitaxel | IHC) | (Poly IHC) | (IHC) | (IHC) | (IHC) | BenefiT |
| | paclitaxel, | Any | Any | T | Any | Any | T |
| | docetaxel, nab- | | | | | | |
| | paclitaxel | | | | | | |
| | nab-paclitaxel | T | Any | F or No | T or No | Any | T |
| | | | | Data | Data | | |
| | paclitaxel, | T | Any | F or No | F | Any | T |
| | docetaxel, nab- | | | Data | | | |
| | paclitaxel | | | | | | |
| | nab-paclitaxel | F or No | T | F or No | T or No | Any | T |
| | | Data | | Data | Data | | |
| | paclitaxel, | F or No | T | F or No | F | Any | T |
| | docetaxel, nab- | Data | | Data | | | |
| | paclitaxel | | | | | | |
| | paclitaxel, | F or No | F | F | T or No | Any | F |
| | docetaxel, nab- | Data | | | Data | | |
| | paclitaxel | | | | | | |
| | paclitaxel, | F or No | F | F or No | F | Any | T |
| | docetaxel, nab- | Data | | Data | | | |
| | paclitaxel | | | | | | |
| | paclitaxel, | F or No | F | No Data | T | Any | F |
| | docetaxel, nab- | Data | | | | | |
| | paclitaxel | | | | | | |
| | nab-paclitaxel | F | F or No | No Data | No Data | Any | F |
| | | | Data | | | | |
| | paclitaxel, | F | No Data | F | T or No | Any | F |
| | docetaxel, nab- | | | | Data | | |
| | paclitaxel | | | | | | |
| | paclitaxel, | F or No | No Data | F or No | F | Any | T |
| | docetaxel, nab- | Data | | Data | | | |
| | paclitaxel | | | | | | |
| | paclitaxel, | F or No | No Data | No Data | T | Any | F |
| | docetaxel, nab- | Data | | | | | |
| | paclitaxel | | | | | | |
| | nab-paclitaxel | No Data | F | No Data | No Data | Any | F |
| | paclitaxel, | No Data | No Data | F | T or No | Any | F |
| | docetaxel, nab- | | | | Data | | |
| | paclitaxel | | | | | | |
| | paclitaxel, | No Data | No Data | No Data | No Data | Any | Indet. |
| | docetaxel, nab- | | | | | | |
| | paclitaxel | | | | | | |

In an embodiment, the invention provides molecular intelligence (MI) profiles that can be used for any lineage of cancer, e.g., for any solid tumor. The MI molecular profiles can be based on assessing the biomarkers using the molecular profiling methods illustrated in FIGS. 33A-B or Table 21. In an embodiment, the molecular intelligence molecular profile for a cancer comprises one or more, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57 or 58, of: ABL1, AKT1, ALK, APC, AR, ATM, BRAF, CDH1, cKIT, cMET, CSF1R, CTNNB1, EGFR, ER, ERBB2, ERBB4, FBXW7, FGFR1, FGFR2, FLT3, GNAT1, GNAQ, GNAS, HER2, HNF1A, HRAS, IDH1, JAK2, JAK3, KDR (VEGFR2), KRAS, MGMT, MLH1, MPL, NOTCH1, NPM1, NRAS, PDGFRA, PGP, PIK3CA, PR, PTEN, PTPN11, RB1, RET, RRM1, SMAD4, SMARCB1, SMO, SPARC, STK11, TLE3, TOP2A, TOPO1, TP53, TS, TUBB3, VHL. The invention further provides a method of selecting a candidate treatment for a cancer comprising assessment of one or more members of the cancer molecular profile using one or more molecular profiling technique presented herein, e.g., ISH (e.g., FISH, CISH), IHC, RT-PCR, expression array, mutation analysis (e.g., NextGen sequencing, Sanger sequencing, pyrosequencing, Fragment analysis (FA, e.g., RFLP), PCR), etc. In one embodiment, ISH is used to assess one or more, e.g., 1 or 2, of: cMET, HER2. Any useful ISH technique can be used. For example, FISH can be used to assess cMET and/or HER2; or CISH can be used to assess cMET and/or HER2. In an embodiment, protein analysis such as IHC is used to assess one or more, e.g., 1, 2, 3, 4, 5, 6, 7, 8, 9, 10, 11, 12, 13, 14, 15 or 16 of: AR, cMET, ER, HER2, MGMT, PGP, PR, PTEN, RRM1, SPARCm, SPARCp, TLE3, TOP2A, TOPO1, TS, TUBB3. “m” and “p” as in SPARC (m/p) refer to IHC performed with monoclonal (“m”) or polyclonal (“p”) primary antibodies. In some embodiments, sequence analysis is used to assess one or more, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44 or 45 of: ABL1, AKT1, ALK, APC, ATM, BRAF, CDH1, cKIT, cMET, CSF1R, CTNNB1, EGFR, ERBB2, ERBB4, FBXW7, FGFR1, FGFR2, FLT3, GNA11, GNAQ, GNAS, HNF1A, HRAS, IDH1, JAK2, JAK3, KDR (VEGFR2), KRAS, MLH1, MPL, NOTCH1, NPM1, NRAS, PDGFRA, PIK3CA, PTEN, PTPN11, RB1, RET, SMAD4, SMARCB1, SMO, STK11, TP53, VHL. For example, the sequence analysis can be performed on one or more, e.g., 1, 2, 3, 4, 5, 6, 7, 8, 9, 10, 11, 12, 13 or 14 of ABL1, APC, BRAF, EGFR, FLT3, GNAQ, IDH1, JAK2, cKIT, KRAS, MPL, NRAS, PDGFRA, VHL. The sequence analysis can also be performed on one or more, e.g., 1, 2, 3, 4, 5, 6, 7, 8, 9, 10, 11, 12, 13, 14 or 15 of ABL1, APC, BRAF, EGFR, FLT3, GNAQ, IDH1, JAK2, cKIT, KRAS, MPL, NPM1, NRAS, PDGFRA, VHL. The sequencing may be performed using Next Generation sequencing technology or other technologies as described herein. For example, methylation of the MGMT promoter region can be assessed using pyrosequencing. The molecular profile can be based on assessing the biomarkers as illustrated in FIGS. 33A-B or Table 21.

In an embodiment, the invention provides a molecular intelligence molecular profile for a cancer comprising analysis of the biomarkers in FIG. 33A, which may be assessed as indicated in the paragraph above and/or as in FIG. 33A or Table 21. For example, the MI profile for a cancer such as a solid tumor may comprise: 1) ISH to assess one or more, e.g., 1 or 2, of: cMET, HER2; 2) IHC to assess one or more, e.g., 1, 2, 3, 4, 5, 6, 7, 8, 9, 10, 11, 12, 13, 14, 15 or 16 of: AR, cMET, ER, HER2, MGMT, PGP, PR, PTEN, RRM1, SPARCm, SPARCp, TLE3, TOP2A, TOPO1, TS, TUBB3; and/or 3) sequence analysis to assess one or more, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33 or 34 of: ABL1, AKT1, ALK, APC, ATM, BRAF, cKIT, cMET, CSF1R, CTNNB1, EGFR, ERBB2, FGFR1, FGFR2, FLT3, GNA11, GNAQ, GNAS, HRAS, IDH1, JAK2, KDR (VEGFR2), KRAS, MLH1, MPL, NOTCH1, NRAS, PDGFRA, PIK3CA, PTEN, RET, SMO, TP53, VHL. In another embodiment, the invention provides a molecular intelligence (MI) PLUS profile for a cancer comprising analysis of the biomarkers in the molecular intelligence (MI) profile and the additional biomarker in FIG. 33B, i.e., 1, 2, 3, 4, 5, 6, 7, 8, 9, 10 or 11 of CDH1, ERBB4, FBXW7, HNF1A, JAK3, NPM1, PTPN11, RB1, SMAD4, SMARCB1 and STK11, which may be assessed as indicated this paragraph and/or as in FIG. 33B or Table 21 below. The invention further provides a report comprising results of the molecular profiling and corresponding candidate treatments that are identified as likely beneficial or likely not beneficial, as further described herein.

Table 21 below presents a view of the information that is reported for a molecular intelligence molecular profile for any cancer, including without limitation a solid tumor, which can be interpreted as described for Table 7 above. The biomarker-treatment associations for the molecular profile for the cancer may comprise those associations in Table 22, which can generally be interpreted as described for Table 8 above.

TABLE 21
Molecular Profile and Report Parameters: Any Solid Tumor (including Glioma)

| Agent(s)/Biomarker Status Reported | Biomarker | Platform |
|---|---|---|
| docetaxel, paclitaxel, nab-paclitaxel | TLE3 | IHC |
| | TUBB3 | IHC |
| | Pgp | IHC |
| | SPARCm | IHC |
| | SPARCp | IHC |
| capecitabine, fluorouracil, pemetrexed | TS | IHC |
| doxorubicin, liposomal-doxorubicin, epirubicin | HER2 | FISH/CISH |
| | TOP2A | IHC |
| | Pgp | IHC |
| irinotecan, topotecan | TOPO1 | IHC |
| gemcitabine | RRM1 | IHC |
| temozolomide, dacarbazine | MGMT | IHC |
| | | (all lineages EXCEPT |
| | | Glioma) |
| | MGMT-Me | Pyrosequencing |
| | | (Glioma ONLY) |
| | IDH1* | NGS |
| abiraterone, bicalutamide, flutamide | AR | IHC |
| fulvestrant, tamoxifen, toremifene, anastrozole, | ER | IHC |
| exemestane, letrozole, megestrol acetate, leuprolide, | PR | IHC |
| goserelin | | |
| trastuzumab, lapatinib, pertuzumab, T-DM1, clinical | HER2 | IHC, FISH/CISH |
| trials | | |
| imatinib | cKIT | NGS |
| | PDGFRA | NGS |
| sunitinib (GIST only) | cKIT | NGS |
| everolimus, temsirolimus, clinical trials | PIK3CA | NGS |
| vandetanib | RET | NGS |
| Clinical Trials | EGFRvIII | Fragment Analysis (FA) |
| | | (Glioma ONLY) |
| Clinical Trials | IDH2 | Sanger Sequencing |
| | | (Glioma ONLY) |
| clinical trials | PTEN | IHC |
| clinical trials | cMET | IHC, FISH/CISH |
| clinical trials | BRAF | NGS |
| clinical trials | KRAS | NGS |
| clinical trials | NRAS | NGS |
| clinical trials | VHL | NGS |
| clinical trials | PTEN | NGS |
| clinical trials | ABL1 | NGS |
| clinical trials | AKT1 | NGS |
| clinical trials | ALK | NGS |
| clinical trials | APC | NGS |
| clinical trials | ATM | NGS |
| clinical trials | CSF1R | NGS |
| clinical trials | CTNNB1 | NGS |
| clinical trials | EGFR | NGS |
| clinical trials | ERBB2 | NGS |
| | (HER2) | |
| clinical trials | FGFR1 | NGS |
| clinical trials | FGFR2 | NGS |
| clinical trials | FLT3 | NGS |
| clinical trials | GNAQ | NGS |
| clinical trials | GNA11 | NGS |
| clinical trials | GNAS | NGS |
| clinical trials | HRAS | NGS |
| clinical trials | JAK2 | NGS |
| clinical trials | KDR | NGS |
| | (VEGFR2) | |
| clinical trials | cMET | NGS |
| clinical trials | MLH1 | NGS |
| clinical trials | MPL | NGS |
| clinical trials | NOTCH1 | NGS |
| clinical trials | SMO | NGS |
| clinical trials | TP53 | NGS |

*IDH1 will only associate with temozolomide, dacarbazine in High Grade Glioma lineage.

In addition to the columns in the tables above, Table 22 provides a predicted benefit level and an evidence level, and list of references for each biomarker-drug association rule in the table. The benefit level is ranked from 1-5, wherein the levels indicate the predicted strength of the biomarker-drug association based on the indicated evidence. All relevant published studies were evaluated using the U.S. Preventive Services Task Force (“USPSTF”) grading scheme for study design and validity. See, e.g., www.uspreventiveservicestaskforce.org/uspstf/grades.htm. The benefit level in the table (“Bene. Level”) corresponds to the following:

1: Expected benefit.

2: Expected reduced benefit.

3: Expected lack of benefit.

4: No data is available.

5: Data is available but no expected benefit or lack of benefit reported because the biomarker in this case is the not principal driver of that specific rule.

The evidence level in the table (“Evid. Level”) corresponds to the following:

1: Very high level of evidence. For example, the treatment comprises the standard of care.

2: High level of evidence but perhaps insufficient to be considered for standard of care.

3: Weaker evidence—fewer publications or clinical studies, or perhaps some controversial evidence.

Abbreviations used in Table 22 include: Bene. (Benefit); Evid. (Evidence); Indet. (Indeterminate); Equiv. (Equivocal); Seq. (Sequencing). In the column “Drugs,” under the section for Taxanes, the following abbreviations are used: PDN (paclitaxel, docetaxel, nab-paclitaxel) and N (nab-paclitaxel).

The column “Partial Report Overall Benefit” in Table 22 is to make drug association in a preliminary molecular profiling report when all the biomarker assessment results may not be ready. For example, a preliminary report may be produced when requested by the treating physician. Interpretation of benefit of lack of benefit of the various drugs is more cautious in these scenarios to avoid potential change in drug association from benefit or lack of benefit or vice versa between the preliminary report and a final report that is produced when all biomarker results become available. Hence you will see some indeterminate scenarios.

TABLE 22
Solid Tumor Drug-Biomarker Associations

| | | | | | | | | | | | | | | | | | | | | | | | Partial |
|---|---|---|---|---|---|---|---|---|---|---|---|---|---|---|---|---|---|---|---|---|---|---|---|
| | | Bio- | | | | Bio- | | | | Bio- | | | | Bio- | | | | Bio- | | | | | Report |
| Class | | marker | Bene. | Evid. | Ref. | marker | Bene. | Evid. | Ref. | marker | Bene. | Evid. | Ref. | marker | Bene. | Evid. | Ref. | marker | Bene. | Evid. | Ref. | Overall | Overall |
| of Drugs | Drugs | Result | Level | Level | No. | Result | Level | Level | No. | Result | Level | Level | No. | Result | Level | Level | No. | Result | Level | Level | No. | Bene. | Bene. |
| | | | | | | | | | | | | | | | | | | | | | | | Partial |
| | | RRM1 | | | | | | | | | | | | | | | | | | | | | Report |
| Anti- | | Negative | Bene. | Evid. | | | | | | | | | | | | | | | | | | Overall | Overall |
| metabolites | gemcitabine | (IHC) | Level | Level | 1 | | | | | | | | | | | | | | | | | Bene. | Bene. |
| | | T | 1 | 2 | | | | | | | | | | | | | | | | | | T | T |
| | | F | 3 | 2 | | | | | | | | | | | | | | | | | | F | F |
| | | No Data | 4 | | | | | | | | | | | | | | | | | | | Indet. | Indet. |
| | | | | | | | | | | | | | | | | | | | | | | | Partial |
| | fluorouracil, | TS | | | | | | | | | | | | | | | | | | | | | Report |
| Anti- | capecitabine, | Negative | Bene. | Evid. | | | | | | | | | | | | | | | | | | Overall | Overall |
| metabolites | pemetrexed | (IHC) | Level | Level | 2 | | | | | | | | | | | | | | | | | Bene. | Bene. |
| | | T | 1 | 2 | | | | | | | | | | | | | | | | | | T | T |
| | | F | 3 | 2 | | | | | | | | | | | | | | | | | | F | F |
| | | No Data | 4 | | | | | | | | | | | | | | | | | | | Indet. | Indet. |
| | | | | | | | | | | | | | | | | | | | | | | | Partial |
| | | TOPO1 | | | | | | | | | | | | | | | | | | | | | Report |
| Topo1 | irinotecan, | Positive | Bene. | Evid. | | | | | | | | | | | | | | | | | | Overall | Overall |
| inhibitors | topotecan | (IHC) | Level | Level | 3 | | | | | | | | | | | | | | | | | Bene. | Bene. |
| | | T | 1 | 2 | | | | | | | | | | | | | | | | | | T | T |
| | | F | 3 | 2 | | | | | | | | | | | | | | | | | | F | F |
| | | No Data | 4 | | | | | | | | | | | | | | | | | | | Indet. | Indet. |
| | | | | | | | | | | | | | | | | | | | | | | | Partial |
| | | MGMT | | | | | | | | | | | | | | | | | | | | | Report |
| Alkylating | temozolomide, | Negative | Bene. | Evid. | | | | | | | | | | | | | | | | | | Overall | Overall |
| agents | dacarbazine | (IHC) | Level | Level | 4 | | | | | | | | | | | | | | | | | Bene. | Bene. |
| | | T | 1 | 2 | | | | | | | | | | | | | | | | | | T | T |
| | | F | 3 | 2 | | | | | | | | | | | | | | | | | | F | F |
| | | No Data | 4 | | | | | | | | | | | | | | | | | | | Indet. | Indet. |
| | | | | | | | | | | | | | | | | | | | | | | | Partial |
| | bicalutamide, | AR | | | | | | | | | | | | | | | | | | | | | Report |
| Anti- | flutamide, | Positive | Bene. | Evid. | | | | | | | | | | | | | | | | | | Overall | Overall |
| androgens | abiraterone | (IHC) | Level | Level | 5 | | | | | | | | | | | | | | | | | Bene. | Bene. |
| | | T | 1 | 2 | | | | | | | | | | | | | | | | | | T | T |
| | | F | 3 | 2 | | | | | | | | | | | | | | | | | | F | F |
| | | No Data | 4 | | | | | | | | | | | | | | | | | | | Indet. | Indet. |
| | tamoxifen, | | | | | | | | | | | | | | | | | | | | | | |
| | toremifene, | | | | | | | | | | | | | | | | | | | | | | |
| | fulvestrant, | | | | | | | | | | | | | | | | | | | | | | |
| | letrozole, | | | | | | | | | | | | | | | | | | | | | | |
| | anastrozole, | | | | | | | | | | | | | | | | | | | | | | |
| | exemestane, | | | | | | | | | | | | | | | | | | | | | | |
| | megestrol | | | | | | | | | | | | | | | | | | | | | | Partial |
| | acetate, | ER | | | | PR | | | | | | | | | | | | | | | | | Report |
| Hormonal | leuprolide, | Positive | Bene. | Evid. | | Positive | Bene. | Evid. | | | | | | | | | | | | | | Overall | Overall |
| Agents | goserelin | (IHC) | Level | Level | 6 | (IHC) | Level | Level | 7 | | | | | | | | | | | | | Bene. | Bene. |
| | | T | 1 | 1 | | T | 1 | 1 | | | | | | | | | | | | | | T | T |
| | | T | 1 | 1 | | F | 2 | 1 | | | | | | | | | | | | | | T | T |
| | | T | 1 | 1 | | No Data | 4 | | | | | | | | | | | | | | | T | T |
| | | F | 2 | 1 | | T | 1 | 1 | | | | | | | | | | | | | | T | T |
| | | F | 3 | 1 | | F | 3 | 1 | | | | | | | | | | | | | | F | F |
| | | F | 3 | 1 | | No Data | 4 | | | | | | | | | | | | | | | Indet. | Indet. |
| | | No Data | 4 | | | T | 1 | 1 | | | | | | | | | | | | | | T | T |
| | | No Data | 4 | | | F | 3 | 1 | | | | | | | | | | | | | | Indet. | Indet. |
| | | No Data | 4 | | | No Data | 4 | | | | | | | | | | | | | | | Indet. | Indet. |
| | | | | | | | | | | | | | | | | | | | | | | | Partial |
| | | HER2 | | | | HER2 | | | | | | | | | | | | | | | | | Report |
| | | Positive | Bene. | Evid. | | Amplified | Bene. | Evid. | | | | | | | | | | | | | | Overall | Overall |
| TKI | lapatinib | (IHC) | Level | Level | 8 | (ISH) | Level | Level | 9 | | | | | | | | | | | | | Bene. | Bene. |
| | | T | 1 | 1 | | T | 1 | 1 | | | | | | | | | | | | | | T | T |
| | | T | 1 | 1 | | F | 5 | | | | | | | | | | | | | | | T | T |
| | | T | 1 | 1 | | Equiv. | 1 | 1 | | | | | | | | | | | | | | T | T |
| | | | | | | High | | | | | | | | | | | | | | | | | |
| | | T | 1 | 1 | | Equiv. | 5 | | | | | | | | | | | | | | | T | T |
| | | | | | | Low | | | | | | | | | | | | | | | | | |
| | | T | 1 | 1 | | No Data | 4 | | | | | | | | | | | | | | | T | T |
| | | F | 5 | | | T | 1 | 1 | | | | | | | | | | | | | | T | T |
| | | F | 3 | 1 | | F | 3 | 1 | | | | | | | | | | | | | | F | F |
| | | F | 5 | | | Equiv. | 1 | 1 | | | | | | | | | | | | | | T | T |
| | | | | | | High | | | | | | | | | | | | | | | | | |
| | | F | 3 | 1 | | Equiv. | 3 | 1 | | | | | | | | | | | | | | F | F |
| | | | | | | Low | | | | | | | | | | | | | | | | | |
| | | F | 3 | 1 | | No Data | 4 | | | | | | | | | | | | | | | Indet. | Indet. |
| | | Equiv. | 5 | | | T | 1 | 1 | | | | | | | | | | | | | | T | T |
| | | Equiv. | 5 | | | F | 3 | 1 | | | | | | | | | | | | | | F | F |
| | | Equiv. | 5 | | | Equiv. | 1 | 1 | | | | | | | | | | | | | | T | T |
| | | | | | | High | | | | | | | | | | | | | | | | | |
| | | Equiv. | 5 | | | Equiv. | 3 | 1 | | | | | | | | | | | | | | F | F |
| | | | | | | Low | | | | | | | | | | | | | | | | | |
| | | Equiv. | 5 | | | No Data | 4 | | | | | | | | | | | | | | | Indet. | Indet. |
| | | No Data | 4 | | | T | 1 | 1 | | | | | | | | | | | | | | T | T |
| | | No Data | 4 | | | F | 3 | 1 | | | | | | | | | | | | | | Indet. | Indet. |
| | | No Data | 4 | | | Equiv. | 1 | 1 | | | | | | | | | | | | | | T | T |
| | | | | | | High | | | | | | | | | | | | | | | | | |
| | | No Data | 4 | | | Equiv. | 3 | 1 | | | | | | | | | | | | | | Indet. | Indet. |
| | | | | | | Low | | | | | | | | | | | | | | | | | |
| | | No Data | 4 | | | No Data | 4 | | | | | | | | | | | | | | | Indet. | Indet. |
| | trastuzumab, | | | | | | | | | | | | | | | | | | | | | | |
| Mono- | pertuzumab, | | | | | | | | | | | | | | | | | | | | | | |
| clonal | ado- | | | | | | | | | | | | | | | | | | | | | | Partial |
| antibodies | trastuzumab | HER2 | | | | HER2 | | | | | | | | | | | | | | | | | Report |
| (Her2- | emtansine | Positive | Bene. | Evid. | | Amplified | Bene. | Evid. | | | | | | | | | | | | | | Overall | Overall |
| Targeted) | (T-DM1) | (IHC) | Level | Level | 10 | (ISH) | Level | Level | 11 | | | | | | | | | | | | | Bene. | Bene. |
| | | T | 1 | 1 | | T | 1 | 1 | | | | | | | | | | | | | | T | T |
| | | T | 1 | 1 | | F | 5 | | | | | | | | | | | | | | | T | T |
| | | T | 1 | 1 | | Equiv. low | 5 | | | | | | | | | | | | | | | T | T |
| | | T | 1 | 1 | | Equiv. high | 1 | 1 | | | | | | | | | | | | | | T | T |
| | | T | 1 | 1 | | No Data | 4 | | | | | | | | | | | | | | | T | T |
| | | F | 5 | | | T | 1 | 1 | | | | | | | | | | | | | | T | T |
| | | F | 3 | 1 | | F | 3 | 1 | | | | | | | | | | | | | | F | F |
| | | F | 3 | 1 | | Equiv. low | 3 | 1 | | | | | | | | | | | | | | F | F |
| | | F | 5 | | | Equiv. high | 1 | 1 | | | | | | | | | | | | | | T | T |
| | | F | 3 | 1 | | No Data | 4 | | | | | | | | | | | | | | | Indet. | Indet. |
| | | Equiv. | 5 | | | T | 1 | 1 | | | | | | | | | | | | | | T | T |
| | | Equiv. | 5 | | | F | 3 | 1 | | | | | | | | | | | | | | F | F |
| | | Equiv. | 5 | | | Equiv. low | 3 | 1 | | | | | | | | | | | | | | F | F |
| | | Equiv. | 5 | | | Equiv. high | 1 | 1 | | | | | | | | | | | | | | T | T |
| | | Equiv. | 5 | | | No Data | 4 | | | | | | | | | | | | | | | Indet. | Indet. |
| | | No Data | 4 | | | T | 1 | 1 | | | | | | | | | | | | | | T | T |
| | | No Data | 4 | | | F | 3 | 1 | | | | | | | | | | | | | | Indet. | Indet. |
| | | No Data | 4 | | | Equiv. low | 3 | 1 | | | | | | | | | | | | | | Indet. | Indet. |
| | | No Data | 4 | | | Equiv. high | 1 | 1 | | | | | | | | | | | | | | T | T |
| | | No Data | 4 | | | No Data | 4 | | | | | | | | | | | | | | | Indet. | Indet. |
| Anthra- | doxorubicin, | | | | | | | | | | | | | | | | | | | | | | Partial |
| cyclines | liposomal- | TOP2A | | | | Her2 | | | | TOP2A | | | | PGP | | | | | | | | | Report |
| and related | doxorubicin, | Amplified | Bene. | Evid. | | Amplified | Bene. | Evid. | | Positive | Bene. | Evid. | | Positive | Bene. | Evid. | | | | | | Overall | Overall |
| substances | epirubicin | (ISH) | Level | Level | 12 | (ISH) | Level | Level | 13 | (IHC) | Level | Level | 14 | (IHC) | Level | Level | 15 | | | | | Bene. | Bene. |
| | | T | 1 | 1 | | T | 1 | 1 | | T | 1 | 2 | | T | 2 | 2 | | | | | | T | T |
| | | T | 1 | 1 | | T | 1 | 1 | | T | 1 | 2 | | F | 1 | 2 | | | | | | T | T |
| | | T | 1 | 1 | | T | 1 | 1 | | T | 1 | 2 | | No Data | 4 | | | | | | | T | T |
| | | T | 1 | 1 | | T | 1 | 1 | | F | 2 | 2 | | T | 2 | 2 | | | | | | T | T |
| | | T | 1 | 1 | | T | 1 | 1 | | F | 2 | 2 | | F | 1 | 2 | | | | | | T | T |
| | | T | 1 | 1 | | T | 1 | 1 | | F | 2 | 2 | | No Data | 4 | | | | | | | T | T |
| | | T | 1 | 1 | | T | 1 | 1 | | No Data | 4 | | | T | 2 | 2 | | | | | | T | T |
| | | T | 1 | 1 | | T | 1 | 1 | | No Data | 4 | | | F | 1 | 2 | | | | | | T | T |
| | | T | 1 | 1 | | T | 1 | 1 | | No Data | 4 | | | No Data | 4 | | | | | | | T | T |
| | | T | 1 | 1 | | F | 2 | 2 | | T | 1 | 2 | | T | 2 | 2 | | | | | | T | T |
| | | T | 1 | 1 | | F | 2 | 2 | | T | 1 | 2 | | F | 1 | 2 | | | | | | T | T |
| | | T | 1 | 1 | | F | 2 | 2 | | T | 1 | 2 | | No Data | 4 | | | | | | | T | T |
| | | T | 1 | 1 | | F | 2 | 1 | | F | 2 | 2 | | T | 2 | 2 | | | | | | T | T |
| | | T | 1 | 1 | | F | 2 | 1 | | F | 2 | 2 | | F | 1 | 2 | | | | | | T | T |
| | | T | 1 | 1 | | F | 2 | 1 | | F | 2 | 2 | | No Data | 4 | | | | | | | T | T |
| | | T | 1 | 1 | | F | 2 | 1 | | No Data | 4 | | | T | 2 | 2 | | | | | | T | T |
| | | T | 1 | 1 | | F | 2 | 1 | | No Data | 4 | | | F | 1 | 2 | | | | | | T | T |
| | | T | 1 | 1 | | F | 2 | 1 | | No Data | 4 | | | No Data | 4 | | | | | | | T | T |
| | | T | 1 | 1 | | No Data | 4 | | | T | 1 | 2 | | T | 2 | 2 | | | | | | T | T |
| | | T | 1 | 1 | | No Data | 4 | | | T | 1 | 2 | | F | 1 | 2 | | | | | | T | T |
| | | T | 1 | 1 | | No Data | 4 | | | T | 1 | 2 | | No Data | 4 | | | | | | | T | T |
| | | T | 1 | 1 | | No Data | 4 | | | F | 2 | 2 | | T | 2 | 2 | | | | | | T | T |
| | | T | 1 | 1 | | No Data | 4 | | | F | 2 | 2 | | F | 1 | 2 | | | | | | T | T |
| | | T | 1 | 1 | | No Data | 4 | | | F | 2 | 2 | | No Data | 4 | | | | | | | T | T |
| | | T | 1 | 1 | | No Data | 4 | | | No Data | 4 | | | T | 2 | 2 | | | | | | T | T |
| | | T | 1 | 1 | | No Data | 4 | | | No Data | 4 | | | F | 1 | 2 | | | | | | T | T |
| | | T | 1 | 1 | | No Data | 4 | | | No Data | 4 | | | No Data | 4 | | | | | | | T | T |
| | | T | 1 | 1 | | Equiv. high | 1 | 1 | | T | 1 | 2 | | T | 2 | 2 | | | | | | T | T |
| | | T | 1 | 1 | | Equiv. high | 1 | 1 | | T | 1 | 2 | | F | 1 | 2 | | | | | | T | T |
| | | T | 1 | 1 | | Equiv. high | 1 | 1 | | T | 1 | 2 | | No Data | 4 | | | | | | | T | T |
| | | T | 1 | 1 | | Equiv. high | 1 | 1 | | F | 2 | 2 | | T | 2 | 2 | | | | | | T | T |
| | | T | 1 | 1 | | Equiv. high | 1 | 1 | | F | 2 | 2 | | F | 1 | 2 | | | | | | T | T |
| | | T | 1 | 1 | | Equiv. high | 1 | 1 | | F | 2 | 2 | | No Data | 4 | | | | | | | T | T |
| | | T | 1 | 1 | | Equiv. high | 1 | 1 | | No Data | 4 | | | T | 2 | 2 | | | | | | T | T |
| | | T | 1 | 1 | | Equiv. high | 1 | 1 | | No Data | 4 | | | F | 1 | 2 | | | | | | T | T |
| | | T | 1 | 1 | | Equiv. high | 1 | 1 | | No Data | 4 | | | No Data | 4 | | | | | | | T | T |
| | | T | 1 | 1 | | Equiv. low | 2 | 2 | | T | 1 | 2 | | T | 2 | 2 | | | | | | T | T |
| | | T | 1 | 1 | | Equiv. low | 2 | 2 | | T | 1 | 2 | | F | 1 | 2 | | | | | | T | T |
| | | T | 1 | 1 | | Equiv. low | 2 | 2 | | T | 1 | 2 | | No Data | 4 | | | | | | | T | T |
| | | T | 1 | 1 | | Equiv. low | 2 | 1 | | F | 2 | 2 | | T | 2 | 2 | | | | | | T | T |
| | | T | 1 | 1 | | Equiv. low | 2 | 1 | | F | 2 | 2 | | F | 1 | 2 | | | | | | T | T |
| | | T | 1 | 1 | | Equiv. low | 2 | 1 | | F | 2 | 2 | | No Data | 4 | | | | | | | T | T |
| | | T | 1 | 1 | | Equiv. low | 2 | 1 | | No Data | 4 | | | T | 2 | 2 | | | | | | T | T |
| | | T | 1 | 1 | | Equiv. low | 2 | 1 | | No Data | 4 | | | F | 1 | 2 | | | | | | T | T |
| | | T | 1 | 1 | | Equiv. low | 2 | 1 | | No Data | 4 | | | No Data | 4 | | | | | | | T | T |
| | | F | 2 | 2 | | T | 1 | 1 | | T | 1 | 2 | | T | 2 | 2 | | | | | | T | T |
| | | F | 2 | 2 | | T | 1 | 1 | | T | 1 | 2 | | F | 1 | 2 | | | | | | T | T |
| | | F | 2 | 2 | | T | 1 | 1 | | T | 1 | 2 | | No Data | 4 | | | | | | | T | T |
| | | F | 2 | 1 | | T | 1 | 1 | | F | 2 | 2 | | T | 2 | 2 | | | | | | T | T |
| | | F | 2 | 1 | | T | 1 | 1 | | F | 2 | 2 | | F | 1 | 2 | | | | | | T | T |
| | | F | 2 | 1 | | T | 1 | 1 | | F | 2 | 2 | | No Data | 4 | | | | | | | T | T |
| | | F | 2 | 1 | | T | 1 | 1 | | No Data | 4 | | | T | 2 | 2 | | | | | | T | T |
| | | F | 2 | 1 | | T | 1 | 1 | | No Data | 4 | | | F | 1 | 2 | | | | | | T | T |
| | | F | 2 | 1 | | T | 1 | 1 | | No Data | 4 | | | No Data | 4 | | | | | | | T | T |
| | | F | 2 | 2 | | F | 2 | 2 | | T | 1 | 2 | | T | 2 | 2 | | | | | | T | T |
| | | F | 2 | 2 | | F | 2 | 2 | | T | 1 | 2 | | F | 1 | 2 | | | | | | T | T |
| | | F | 2 | 2 | | F | 2 | 2 | | T | 1 | 2 | | No Data | 4 | | | | | | | T | T |
| | | F | 3 | 1 | | F | 3 | 1 | | F | 3 | 2 | | T | 3 | 2 | | | | | | F | F |
| | | F | 3 | 1 | | F | 3 | 1 | | F | 3 | 2 | | F | 2 | 2 | | | | | | F | F |
| | | F | 3 | 1 | | F | 3 | 1 | | F | 3 | 2 | | No Data | 4 | | | | | | | F | F |
| | | F | 3 | 1 | | F | 3 | 1 | | No Data | 4 | | | T | 3 | 2 | | | | | | F | Indet. |
| | | F | 3 | 1 | | F | 3 | 1 | | No Data | 4 | | | F | 2 | 2 | | | | | | F | Indet. |
| | | F | 3 | 1 | | F | 3 | 1 | | No Data | 4 | | | No Data | 4 | | | | | | | F | Indet. |
| | | F | 2 | 2 | | No Data | 4 | | | T | 1 | 2 | | T | 2 | 2 | | | | | | T | T |
| | | F | 2 | 2 | | No Data | 4 | | | T | 1 | 2 | | F | 1 | 2 | | | | | | T | T |
| | | F | 2 | 2 | | No Data | 4 | | | T | 1 | 2 | | No Data | 4 | | | | | | | T | T |
| | | F | 3 | 1 | | No Data | 4 | | | F | 3 | 2 | | T | 3 | 2 | | | | | | F | Indet. |
| | | F | 3 | 1 | | No Data | 4 | | | F | 3 | 2 | | F | 2 | 2 | | | | | | F | Indet. |
| | | F | 3 | 1 | | No Data | 4 | | | F | 3 | 2 | | No Data | 4 | | | | | | | F | Indet. |
| | | F | 3 | 1 | | No Data | 4 | | | No Data | 4 | | | T | 3 | 2 | | | | | | F | Indet. |
| | | F | 3 | 1 | | No Data | 4 | | | No Data | 4 | | | F | 2 | 2 | | | | | | F | Indet. |
| | | F | 3 | 1 | | No Data | 4 | | | No Data | 4 | | | No Data | 4 | | | | | | | F | Indet. |
| | | F | 2 | 2 | | Equiv. high | 1 | 1 | | T | 1 | 2 | | T | 2 | 2 | | | | | | T | T |
| | | F | 2 | 2 | | Equiv. high | 1 | 1 | | T | 1 | 2 | | F | 1 | 2 | | | | | | T | T |
| | | F | 2 | 2 | | Equiv. high | 1 | 1 | | T | 1 | 2 | | No Data | 4 | | | | | | | T | T |
| | | F | 2 | 1 | | Equiv. high | 1 | 1 | | F | 2 | 2 | | T | 2 | 2 | | | | | | T | T |
| | | F | 2 | 1 | | Equiv. high | 1 | 1 | | F | 2 | 2 | | F | 1 | 2 | | | | | | T | T |
| | | F | 2 | 1 | | Equiv. high | 1 | 1 | | F | 2 | 2 | | No Data | 4 | | | | | | | T | T |
| | | F | 2 | 1 | | Equiv. high | 1 | 1 | | No Data | 4 | | | T | 2 | 2 | | | | | | T | T |
| | | F | 2 | 1 | | Equiv. high | 1 | 1 | | No Data | 4 | | | F | 1 | 2 | | | | | | T | T |
| | | F | 2 | 1 | | Equiv. high | 1 | 1 | | No Data | 4 | | | No Data | 4 | | | | | | | T | T |
| | | F | 2 | 2 | | Equiv. low | 2 | 2 | | T | 1 | 2 | | T | 2 | 2 | | | | | | T | T |
| | | F | 2 | 2 | | Equiv. low | 2 | 2 | | T | 1 | 2 | | F | 1 | 2 | | | | | | T | T |
| | | F | 2 | 2 | | Equiv. low | 2 | 2 | | T | 1 | 2 | | No Data | 4 | | | | | | | T | T |
| | | F | 3 | 1 | | Equiv. low | 3 | 1 | | F | 3 | 2 | | T | 3 | 2 | | | | | | F | F |
| | | F | 3 | 1 | | Equiv. low | 3 | 1 | | F | 3 | 2 | | F | 2 | 2 | | | | | | F | F |
| | | F | 3 | 1 | | Equiv. low | 3 | 1 | | F | 3 | 2 | | No Data | 4 | | | | | | | F | F |
| | | F | 3 | 1 | | Equiv. low | 3 | 1 | | No Data | 4 | | | T | 3 | 2 | | | | | | F | Indet. |
| | | F | 3 | 1 | | Equiv. low | 3 | 1 | | No Data | 4 | | | F | 2 | 2 | | | | | | F | Indet. |
| | | F | 3 | 1 | | Equiv. low | 3 | 1 | | No Data | 4 | | | No Data | 4 | | | | | | | F | Indet. |
| | | No Data | 4 | | | T | 1 | 1 | | T | 1 | 2 | | T | 2 | 2 | | | | | | T | T |
| | | No Data | 4 | | | T | 1 | 1 | | T | 1 | 2 | | F | 1 | 2 | | | | | | T | T |
| | | No Data | 4 | | | T | 1 | 1 | | T | 1 | 2 | | No Data | 4 | | | | | | | T | T |
| | | No Data | 4 | | | T | 1 | 1 | | F | 2 | 2 | | T | 2 | 2 | | | | | | T | T |
| | | No Data | 4 | | | T | 1 | 1 | | F | 2 | 2 | | F | 1 | 2 | | | | | | T | T |
| | | No Data | 4 | | | T | 1 | 1 | | F | 2 | 2 | | No Data | 4 | | | | | | | T | T |
| | | No Data | 4 | | | T | 1 | 1 | | No Data | 4 | | | T | 2 | 2 | | | | | | T | T |
| | | No Data | 4 | | | T | 1 | 1 | | No Data | 4 | | | F | 1 | 2 | | | | | | T | T |
| | | No Data | 4 | | | T | 1 | 1 | | No Data | 4 | | | No Data | 4 | | | | | | | T | T |
| | | No Data | 4 | | | F | 2 | 2 | | T | 1 | 2 | | T | 2 | 2 | | | | | | T | T |
| | | No Data | 4 | | | F | 2 | 2 | | T | 1 | 2 | | F | 1 | 2 | | | | | | T | T |
| | | No Data | 4 | | | F | 2 | 2 | | T | 1 | 2 | | No Data | 4 | | | | | | | T | T |
| | | No Data | 4 | | | F | 3 | 1 | | F | 3 | 2 | | T | 3 | 2 | | | | | | F | Indet. |
| | | No Data | 4 | | | F | 3 | 1 | | F | 3 | 2 | | F | 2 | 2 | | | | | | F | Indet. |
| | | No Data | 4 | | | F | 3 | 1 | | F | 3 | 2 | | No Data | 4 | | | | | | | F | Indet. |
| | | No Data | 4 | | | F | 3 | 1 | | No Data | 4 | | | T | 3 | 2 | | | | | | F | Indet. |
| | | No Data | 4 | | | F | 3 | 1 | | No Data | 4 | | | F | 2 | 2 | | | | | | F | Indet. |
| | | No Data | 4 | | | F | 3 | 1 | | No Data | 4 | | | No Data | 4 | | | | | | | F | Indet. |
| | | No Data | 4 | | | No Data | 4 | | | T | 1 | 2 | | T | 2 | 2 | | | | | | T | T |
| | | No Data | 4 | | | No Data | 4 | | | T | 1 | 2 | | F | 1 | 2 | | | | | | T | T |
| | | No Data | 4 | | | No Data | 4 | | | T | 1 | 2 | | No Data | 4 | | | | | | | T | T |
| | | No Data | 4 | | | No Data | 4 | | | F | 3 | 2 | | T | 3 | 2 | | | | | | F | Indet. |
| | | No Data | 4 | | | No Data | 4 | | | F | 3 | 2 | | F | 2 | 2 | | | | | | F | Indet. |
| | | No Data | 4 | | | No Data | 4 | | | F | 3 | 2 | | No Data | 4 | | | | | | | F | Indet. |
| | | No Data | 4 | | | No Data | 4 | | | No Data | 4 | | | T | 3 | 2 | | | | | | F | Indet. |
| | | No Data | 4 | | | No Data | 4 | | | No Data | 4 | | | F | 1 | 2 | | | | | | T | Indet. |
| | | No Data | 4 | | | No Data | 4 | | | No Data | 4 | | | No Data | 4 | | | | | | | Indet. | Indet. |
| | | No Data | 4 | | | Equiv. high | 1 | 1 | | T | 1 | 2 | | T | 2 | 2 | | | | | | T | T |
| | | No Data | 4 | | | Equiv. high | 1 | 1 | | T | 1 | 2 | | F | 1 | 2 | | | | | | T | T |
| | | No Data | 4 | | | Equiv. high | 1 | 1 | | T | 1 | 2 | | No Data | 4 | | | | | | | T | T |
| | | No Data | 4 | | | Equiv. high | 1 | 1 | | F | 2 | 2 | | T | 2 | 2 | | | | | | T | T |
| | | No Data | 4 | | | Equiv. high | 1 | 1 | | F | 2 | 2 | | F | 1 | 2 | | | | | | T | T |
| | | No Data | 4 | | | Equiv. high | 1 | 1 | | F | 2 | 2 | | No Data | 4 | | | | | | | T | T |
| | | No Data | 4 | | | Equiv. high | 1 | 1 | | No Data | 4 | | | T | 2 | 2 | | | | | | T | T |
| | | No Data | 4 | | | Equiv. high | 1 | 1 | | No Data | 4 | | | F | 1 | 2 | | | | | | T | T |
| | | No Data | 4 | | | Equiv. high | 1 | 1 | | No Data | 4 | | | No Data | 4 | | | | | | | T | T |
| | | No Data | 4 | | | Equiv. low | 2 | 2 | | T | 1 | 2 | | T | 2 | 2 | | | | | | T | T |
| | | No Data | 4 | | | Equiv. low | 2 | 2 | | T | 1 | 2 | | F | 1 | 2 | | | | | | T | T |
| | | No Data | 4 | | | Equiv. low | 2 | 2 | | T | 1 | 2 | | No Data | 4 | | | | | | | T | T |
| | | No Data | 4 | | | Equiv. low | 3 | 1 | | F | 3 | 2 | | T | 3 | 2 | | | | | | F | Indet. |
| | | No Data | 4 | | | Equiv. low | 3 | 1 | | F | 3 | 2 | | F | 2 | 2 | | | | | | F | Indet. |
| | | No Data | 4 | | | Equiv. low | 3 | 1 | | F | 3 | 2 | | No Data | 4 | | | | | | | F | Indet. |
| | | No Data | 4 | | | Equiv. low | 3 | 1 | | No Data | 4 | | | T | 3 | 2 | | | | | | F | Indet. |
| | | No Data | 4 | | | Equiv. low | 3 | 1 | | No Data | 4 | | | F | 2 | 2 | | | | | | F | Indet. |
| | | No Data | 4 | | | Equiv. low | 3 | 1 | | No Data | 4 | | | No Data | 4 | | | | | | | F | Indet. |
| | | | | | | PDGFRA | | | | | | | | | | | | | | | | | |
| | | c-KIT | | | | exon 12 \| | | | | | | | | | | | | | | | | | Partial |
| | | exon11 \| | | | | exon 14 \| | | | | | | | | | | | | | | | | | Report |
| | | exon13 | Bene. | Evid. | | exon 18 | Bene. | Evid. | | | | | | | | | | | | | | Overall | Overall |
| TKI | imatinib | (Seq.) | Level | Level | 16 | (Seq.) | Level | Level | 17 | | | | | | | | | | | | | Bene. | Bene. |
| | | T | 1 | 2 | | T | 1 | 2 | | | | | | | | | | | | | | T | T |
| | | T | 1 | 2 | | F | 5 | | | | | | | | | | | | | | | T | T |
| | | T | 2 | 2 | | D842V | 3 | 2 | | | | | | | | | | | | | | F | F |
| | | T | 1 | 2 | | No Data | 4 | | | | | | | | | | | | | | | T | Indet. |
| | | F | 2 | 2 | | T | 1 | 2 | | | | | | | | | | | | | | T | T |
| | | F | 3 | 2 | | F | 3 | 2 | | | | | | | | | | | | | | Indet. | Indet. |
| | | F | 3 | 2 | | D842V | 3 | 2 | | | | | | | | | | | | | | F | F |
| | | F | 3 | 2 | | No Data | 4 | | | | | | | | | | | | | | | Indet. | Indet. |
| | | V654A | 3 | 2 | | T | 2 | 2 | | | | | | | | | | | | | | F | F |
| | | V654A | 3 | 2 | | F | 3 | 2 | | | | | | | | | | | | | | F | F |
| | | V654A | 3 | 2 | | D842V | 3 | 2 | | | | | | | | | | | | | | F | F |
| | | V654A | 3 | 2 | | No Data | 4 | | | | | | | | | | | | | | | F | F |
| | | exon 14 | 5 | | | T | 1 | 2 | | | | | | | | | | | | | | T | T |
| | | exon 14 | 5 | | | F | 3 | 2 | | | | | | | | | | | | | | Indet. | Indet. |
| | | exon 14 | 5 | | | D842V | 3 | 2 | | | | | | | | | | | | | | F | F |
| | | exon 14 | 5 | | | No Data | 4 | | | | | | | | | | | | | | | Indet. | Indet. |
| | | exon 17 or | 5 | | | T | 1 | 2 | | | | | | | | | | | | | | T | T |
| | | 18 | | | | | | | | | | | | | | | | | | | | | |
| | | exon 17 or | 5 | | | F | 3 | 2 | | | | | | | | | | | | | | Indet. | Indet. |
| | | 18 | | | | | | | | | | | | | | | | | | | | | |
| | | exon 17 or | 5 | | | D842V | 3 | 2 | | | | | | | | | | | | | | F | F |
| | | 18 | | | | | | | | | | | | | | | | | | | | | |
| | | exon 17 or | 5 | | | No Data | 4 | | | | | | | | | | | | | | | Indet. | Indet. |
| | | 18 | | | | | | | | | | | | | | | | | | | | | |
| | | No Data | 4 | | | T | 1 | 2 | | | | | | | | | | | | | | T | Indet. |
| | | No Data | 4 | | | F | 3 | 2 | | | | | | | | | | | | | | Indet. | Indet. |
| | | No Data | 4 | | | D842V | 3 | 2 | | | | | | | | | | | | | | F | F |
| | | No Data | 4 | | | No Data | 4 | | | | | | | | | | | | | | | Indet. | Indet. |
| | | | | | | | | | | | | | | | | | | | | | | | Partial |
| | | ALK | | | | ROS1 | | | | | | | | | | | | | | | | | Report |
| TKI | | Positive | Bene. | Evid. | | Positive | Bene. | Evid. | | | | | | | | | | | | | | Overall | Overall |
| (crizotinib) | crizotinib | (ISH) | Level | Level | 18 | (ISH) | Level | Level | 19 | | | | | | | | | | | | | Bene. | Bene. |
| | | T | 1 | 2 | | T | 1 | 2 | | | | | | | | | | | | | | T | T |
| | | F | 5 | | | T | 1 | 2 | | | | | | | | | | | | | | T | T |
| | | No Data | 4 | | | T | 1 | 2 | | | | | | | | | | | | | | T | T |
| | | T | 1 | 2 | | F | 5 | | | | | | | | | | | | | | | T | T |
| | | F | 3 | 2 | | F | 3 | 2 | | | | | | | | | | | | | | F | F |
| | | No Data | 4 | | | F | 3 | 2 | | | | | | | | | | | | | | Indet. | Indet. |
| | | T | 1 | 2 | | No Data | 4 | | | | | | | | | | | | | | | T | T |
| | | F | 3 | 2 | | No Data | 4 | | | | | | | | | | | | | | | F | Indet. |
| | | No Data | 4 | | | No Data | 4 | | | | | | | | | | | | | | | Indet. | Indet. |
| | | | | | | | | | | | | | | | | | | | | | | | Partial |
| | | PIK3CA | | | | | | | | | | | | | | | | | | | | | Report |
| mTOR | everolimus, | exon20 | Bene. | Evid. | | | | | | | | | | | | | | | | | | Overall | Overall |
| inhibitors | temsirolimus | (Seq.) | Level | Level | 20 | | | | | | | | | | | | | | | | | Bene. | Bene. |
| | | T | 1 | 2 | | | | | | | | | | | | | | | | | | T | T |
| | | F | 3 | 2 | | | | | | | | | | | | | | | | | | Indet. | Indet. |
| | | No Data | 4 | | | | | | | | | | | | | | | | | | | Indet. | Indet. |
| | | | | | | | | | | | | | | | | | | | | | | | Partial |
| | | RET | | | | | | | | | | | | | | | | | | | | | Report |
| TKI (RET- | | Mutated | Bene. | Evid. | | | | | | | | | | | | | | | | | | Overall | Overall |
| targeted) | vandetanib | (Seq.) | Level | Level | 21 | | | | | | | | | | | | | | | | | Bene. | Bene. |
| | | T | 1 | 1 | | | | | | | | | | | | | | | | | | T | T |
| | | F | 5 | | | | | | | | | | | | | | | | | | | Indet. | Indet. |
| | | No Data | 4 | | | | | | | | | | | | | | | | | | | Indet. | Indet. |
| | paclitaxel, | SPARC | | | | SPARC | | | | | | | | | | | | | | | | | Partial |
| | docetaxel, | Positive | | | | Positive | | | | TLE3 | | | | TUBB3 | | | | PGP | | | | | Report |
| | nab- | (Mono | Bene. | Evid. | | (Poly | Bene. | Evid. | | Positive | Bene. | Evid. | | Positive | Bene. | Evid. | | Positive | Bene. | Evid. | | Overall | Overall |
| Taxanes | paclitaxel | IHC) | Level | Level | 22 | IHC) | Level | Level | 22 | (IHC) | Level | Level | 23 | (IHC) | Level | Level | 24 | (IHC) | Level | Level | 25 | Bene. | Bene. |
| | PDN | T | 1 | 2 | | T | 1 | 2 | | T | 1 | 2 | | T | 2 | 2 | | T | 2 | 3 | | T | T |
| | PDN | T | 1 | 2 | | T | 1 | 2 | | T | 1 | 2 | | T | 2 | 2 | | F | 1 | 3 | | T | T |
| | PDN | T | 1 | 2 | | T | 1 | 2 | | T | 1 | 2 | | T | 2 | 2 | | No Data | 4 | | | T | T |
| | PDN | T | 1 | 2 | | T | 1 | 2 | | T | 1 | 2 | | F | 1 | 2 | | T | 2 | 3 | | T | T |
| | PDN | T | 1 | 2 | | T | 1 | 2 | | T | 1 | 2 | | F | 1 | 2 | | F | 1 | 3 | | T | T |
| | PDN | T | 1 | 2 | | T | 1 | 2 | | T | 1 | 2 | | F | 1 | 2 | | No Data | 4 | | | T | T |
| | PDN | T | 1 | 2 | | T | 1 | 2 | | T | 1 | 2 | | No Data | 4 | | | T | 2 | 3 | | T | T |
| | PDN | T | 1 | 2 | | T | 1 | 2 | | T | 1 | 2 | | No Data | 4 | | | F | 1 | 3 | | T | T |
| | PDN | T | 1 | 2 | | T | 1 | 2 | | T | 1 | 2 | | No Data | 4 | | | No Data | 4 | | | T | T |
| | N | T | 1 | 2 | | T | 1 | 2 | | F | 2 | 2 | | T | 2 | 2 | | T | 2 | 3 | | T | T |
| | N | T | 1 | 2 | | T | 1 | 2 | | F | 2 | 2 | | T | 2 | 2 | | F | 1 | 3 | | T | T |
| | N | T | 1 | 2 | | T | 1 | 2 | | F | 2 | 2 | | T | 2 | 2 | | No Data | 4 | | | T | T |
| | PDN | T | 1 | 2 | | T | 1 | 2 | | F | 2 | 2 | | F | 1 | 2 | | T | 2 | 3 | | T | T |
| | PDN | T | 1 | 2 | | T | 1 | 2 | | F | 2 | 2 | | F | 1 | 2 | | F | 1 | 3 | | T | T |
| | PDN | T | 1 | 2 | | T | 1 | 2 | | F | 2 | 2 | | F | 1 | 2 | | No Data | 4 | | | T | T |
| | N | T | 1 | 2 | | T | 1 | 2 | | F | 2 | 2 | | No Data | 4 | | | T | 2 | 3 | | T | Indet. |
| | N | T | 1 | 2 | | T | 1 | 2 | | F | 2 | 2 | | No Data | 4 | | | F | 1 | 3 | | T | Indet. |
| | N | T | 1 | 2 | | T | 1 | 2 | | F | 2 | 2 | | No Data | 4 | | | No Data | 4 | | | T | Indet. |
| | N | T | 1 | 2 | | T | 1 | 2 | | No Data | 4 | | | T | 2 | 2 | | T | 2 | 3 | | T | Indet. |
| | N | T | 1 | 2 | | T | 1 | 2 | | No Data | 4 | | | T | 2 | 2 | | F | 1 | 3 | | T | Indet |
| | N | T | 1 | 2 | | T | 1 | 2 | | No Data | 4 | | | T | 2 | 2 | | No Data | 4 | | | T | Indet. |
| | PDN | T | 1 | 2 | | T | 1 | 2 | | No Data | 4 | | | F | 1 | 2 | | T | 2 | 3 | | T | T |
| | PDN | T | 1 | 2 | | T | 1 | 2 | | No Data | 4 | | | F | 1 | 2 | | F | 1 | 3 | | T | T |
| | PDN | T | 1 | 2 | | T | 1 | 2 | | No Data | 4 | | | F | 1 | 2 | | No Data | 4 | | | T | T |
| | N | T | 1 | 2 | | T | 1 | 2 | | No Data | 4 | | | No Data | 4 | | | T | 2 | 3 | | T | Indet |
| | N | T | 1 | 2 | | T | 1 | 2 | | No Data | 4 | | | No Data | 4 | | | F | 1 | 3 | | T | Indet |
| | N | T | 1 | 2 | | T | 1 | 2 | | No Data | 4 | | | No Data | 4 | | | No Data | 4 | | | T | Indet. |
| | PDN | T | 1 | 2 | | F | 2 | 2 | | T | 1 | 2 | | T | 2 | 2 | | T | 2 | 3 | | T | T |
| | PDN | T | 1 | 2 | | F | 2 | 2 | | T | 1 | 2 | | T | 2 | 2 | | F | 1 | 3 | | T | T |
| | PDN | T | 1 | 2 | | F | 2 | 2 | | T | 1 | 2 | | T | 2 | 2 | | No Data | 4 | | | T | T |
| | PDN | T | 1 | 2 | | F | 2 | 2 | | T | 1 | 2 | | F | 1 | 2 | | T | 2 | 3 | | T | T |
| | PDN | T | 1 | 2 | | F | 2 | 2 | | T | 1 | 2 | | F | 1 | 2 | | F | 1 | 3 | | T | T |
| | PDN | T | 1 | 2 | | F | 2 | 2 | | T | 1 | 2 | | F | 1 | 2 | | No Data | 4 | | | T | T |
| | PDN | T | 1 | 2 | | F | 2 | 2 | | T | 1 | 2 | | No Data | 4 | | | T | 2 | 3 | | T | T |
| | PDN | T | 1 | 2 | | F | 2 | 2 | | T | 1 | 2 | | No Data | 4 | | | F | 1 | 3 | | T | T |
| | PDN | T | 1 | 2 | | F | 2 | 2 | | T | 1 | 2 | | No Data | 4 | | | No Data | 4 | | | T | T |
| | N | T | 1 | 2 | | F | 2 | 2 | | F | 2 | 2 | | T | 2 | 2 | | T | 2 | 3 | | T | T |
| | N | T | 1 | 2 | | F | 2 | 2 | | F | 2 | 2 | | T | 2 | 2 | | F | 1 | 3 | | T | T |
| | N | T | 1 | 2 | | F | 2 | 2 | | F | 2 | 2 | | T | 2 | 2 | | No Data | 4 | | | T | T |
| | PDN | T | 1 | 2 | | F | 2 | 2 | | F | 2 | 2 | | F | 1 | 2 | | T | 2 | 3 | | T | T |
| | PDN | T | 1 | 2 | | F | 2 | 2 | | F | 2 | 2 | | F | 1 | 2 | | F | 1 | 3 | | T | T |
| | PDN | T | 1 | 2 | | F | 2 | 2 | | F | 2 | 2 | | F | 1 | 2 | | No Data | 4 | | | T | T |
| | N | T | 1 | 2 | | F | 2 | 2 | | F | 2 | 2 | | No Data | 4 | | | T | 2 | 3 | | T | Indet. |
| | N | T | 1 | 2 | | F | 2 | 2 | | F | 2 | 2 | | No Data | 4 | | | F | 1 | 3 | | T | Indet. |
| | N | T | 1 | 2 | | F | 2 | 2 | | F | 2 | 2 | | No Data | 4 | | | No Data | 4 | | | T | Indet. |
| | N | T | 1 | 2 | | F | 2 | 2 | | No Data | 4 | | | T | 2 | 2 | | T | 2 | 3 | | T | Indet. |
| | N | T | 1 | 2 | | F | 2 | 2 | | No Data | 4 | | | T | 2 | 2 | | F | 1 | 3 | | T | Indet. |
| | N | T | 1 | 2 | | F | 2 | 2 | | No Data | 4 | | | T | 2 | 2 | | No Data | 4 | | | T | Indet. |
| | PDN | T | 1 | 2 | | F | 2 | 2 | | No Data | 4 | | | F | 1 | 2 | | T | 2 | 3 | | T | T |
| | PDN | T | 1 | 2 | | F | 2 | 2 | | No Data | 4 | | | F | 1 | 2 | | F | 1 | 3 | | T | T |
| | PDN | T | 1 | 2 | | F | 2 | 2 | | No Data | 4 | | | F | 1 | 2 | | No Data | 4 | | | T | T |
| | N | T | 1 | 2 | | F | 2 | 2 | | No Data | 4 | | | No Data | 4 | | | T | 2 | 3 | | T | Indet. |
| | N | T | 1 | 2 | | F | 2 | 2 | | No Data | 4 | | | No Data | 4 | | | F | 1 | 3 | | T | Indet. |
| | N | T | 1 | 2 | | F | 2 | 2 | | No Data | 4 | | | No Data | 4 | | | No Data | 4 | | | T | Indet. |
| | PDN | T | 1 | 2 | | No Data | 4 | | | T | 1 | 2 | | T | 2 | 2 | | T | 2 | 3 | | T | T |
| | PDN | T | 1 | 2 | | No Data | 4 | | | T | 1 | 2 | | T | 2 | 2 | | F | 1 | 3 | | T | T |
| | PDN | T | 1 | 2 | | No Data | 4 | | | T | 1 | 2 | | T | 2 | 2 | | No Data | 4 | | | T | T |
| | PDN | T | 1 | 2 | | No Data | 4 | | | T | 1 | 2 | | F | 1 | 2 | | T | 2 | 3 | | T | T |
| | PDN | T | 1 | 2 | | No Data | 4 | | | T | 1 | 2 | | F | 1 | 2 | | F | 1 | 3 | | T | T |
| | PDN | T | 1 | 2 | | No Data | 4 | | | T | 1 | 2 | | F | 1 | 2 | | No Data | 4 | | | T | T |
| | PDN | T | 1 | 2 | | No Data | 4 | | | T | 1 | 2 | | No Data | 4 | | | T | 2 | 3 | | T | T |
| | PDN | T | 1 | 2 | | No Data | 4 | | | T | 1 | 2 | | No Data | 4 | | | F | 1 | 3 | | T | T |
| | PDN | T | 1 | 2 | | No Data | 4 | | | T | 1 | 2 | | No Data | 4 | | | No Data | 4 | | | T | T |
| | N | T | 1 | 2 | | No Data | 4 | | | F | 2 | 2 | | T | 2 | 2 | | T | 2 | 3 | | T | T |
| | N | T | 1 | 2 | | No Data | 4 | | | F | 2 | 2 | | T | 2 | 2 | | F | 1 | 3 | | T | T |
| | N | T | 1 | 2 | | No Data | 4 | | | F | 2 | 2 | | T | 2 | 2 | | No Data | 4 | | | T | T |
| | PDN | T | 1 | 2 | | No Data | 4 | | | F | 2 | 2 | | F | 1 | 2 | | T | 2 | 3 | | T | T |
| | PDN | T | 1 | 2 | | No Data | 4 | | | F | 2 | 2 | | F | 1 | 2 | | F | 1 | 3 | | T | T |
| | PDN | T | 1 | 2 | | No Data | 4 | | | F | 2 | 2 | | F | 1 | 2 | | No Data | 4 | | | T | T |
| | N | T | 1 | 2 | | No Data | 4 | | | F | 2 | 2 | | No Data | 4 | | | T | 2 | 3 | | T | Indet. |
| | N | T | 1 | 2 | | No Data | 4 | | | F | 2 | 2 | | No Data | 4 | | | F | 1 | 3 | | T | Indet. |
| | N | T | 1 | 2 | | No Data | 4 | | | F | 2 | 2 | | No Data | 4 | | | No Data | 4 | | | T | Indet. |
| | N | T | 1 | 2 | | No Data | 4 | | | No Data | 4 | | | T | 2 | 2 | | T | 2 | 3 | | T | Indet. |
| | N | T | 1 | 2 | | No Data | 4 | | | No Data | 4 | | | T | 2 | 2 | | F | 1 | 3 | | T | Indet. |
| | N | T | 1 | 2 | | No Data | 4 | | | No Data | 4 | | | T | 2 | 2 | | No Data | 4 | | | T | Indet. |
| | PDN | T | 1 | 2 | | No Data | 4 | | | No Data | 4 | | | F | 1 | 2 | | T | 2 | 3 | | T | T |
| | PDN | T | 1 | 2 | | No Data | 4 | | | No Data | 4 | | | F | 1 | 2 | | F | 1 | 3 | | T | T |
| | PDN | T | 1 | 2 | | No Data | 4 | | | No Data | 4 | | | F | 1 | 2 | | No Data | 4 | | | T | T |
| | N | T | 1 | 2 | | No Data | 4 | | | No Data | 4 | | | No Data | 4 | | | T | 2 | 3 | | T | Indet. |
| | N | T | 1 | 2 | | No Data | 4 | | | No Data | 4 | | | No Data | 4 | | | F | 1 | 3 | | T | Indet. |
| | N | T | 1 | 2 | | No Data | 4 | | | No Data | 4 | | | No Data | 4 | | | No Data | 4 | | | T | Indet. |
| | PDN | F | 2 | 2 | | T | 1 | 2 | | T | 1 | 2 | | T | 2 | 2 | | T | 2 | 3 | | T | T |
| | PDN | F | 2 | 2 | | T | 1 | 2 | | T | 1 | 2 | | T | 2 | 2 | | F | 1 | 3 | | T | T |
| | PDN | F | 2 | 2 | | T | 1 | 2 | | T | 1 | 2 | | T | 2 | 2 | | No Data | 4 | | | T | T |
| | PDN | F | 2 | 2 | | T | 1 | 2 | | T | 1 | 2 | | F | 1 | 2 | | T | 2 | 3 | | T | T |
| | PDN | F | 2 | 2 | | T | 1 | 2 | | T | 1 | 2 | | F | 1 | 2 | | F | 1 | 3 | | T | T |
| | PDN | F | 2 | 2 | | T | 1 | 2 | | T | 1 | 2 | | F | 1 | 2 | | No Data | 4 | | | T | T |
| | PDN | F | 2 | 2 | | T | 1 | 2 | | T | 1 | 2 | | No Data | 4 | | | T | 2 | 3 | | T | T |
| | PDN | F | 2 | 2 | | T | 1 | 2 | | T | 1 | 2 | | No Data | 4 | | | F | 1 | 3 | | T | T |
| | PDN | F | 2 | 2 | | T | 1 | 2 | | T | 1 | 2 | | No Data | 4 | | | No Data | 4 | | | T | T |
| | N | F | 2 | 2 | | T | 1 | 2 | | F | 2 | 2 | | T | 2 | 2 | | T | 2 | 3 | | T | T |
| | N | F | 2 | 2 | | T | 1 | 2 | | F | 2 | 2 | | T | 2 | 2 | | F | 1 | 3 | | T | T |
| | N | F | 2 | 2 | | T | 1 | 2 | | F | 2 | 2 | | T | 2 | 2 | | No Data | 4 | | | T | T |
| | PDN | F | 2 | 2 | | T | 1 | 2 | | F | 2 | 2 | | F | 1 | 2 | | T | 2 | 3 | | T | T |
| | PDN | F | 2 | 2 | | T | 1 | 2 | | F | 2 | 2 | | F | 1 | 2 | | F | 1 | 3 | | T | T |
| | PDN | F | 2 | 2 | | T | 1 | 2 | | F | 2 | 2 | | F | 1 | 2 | | No Data | 4 | | | T | T |
| | N | F | 2 | 2 | | T | 1 | 2 | | F | 2 | 2 | | No Data | 4 | | | T | 2 | 3 | | T | Indet. |
| | N | F | 2 | 2 | | T | 1 | 2 | | F | 2 | 2 | | No Data | 4 | | | F | 1 | 3 | | T | Indet. |
| | N | F | 2 | 2 | | T | 1 | 2 | | F | 2 | 2 | | No Data | 4 | | | No Data | 4 | | | T | Indet. |
| | N | F | 2 | 2 | | T | 1 | 2 | | No Data | 4 | | | T | 2 | 2 | | T | 2 | 3 | | T | Indet. |
| | N | F | 2 | 2 | | T | 1 | 2 | | No Data | 4 | | | T | 2 | 2 | | F | 1 | 3 | | T | Indet. |
| | N | F | 2 | 2 | | T | 1 | 2 | | No Data | 4 | | | T | 2 | 2 | | No Data | 4 | | | T | Indet. |
| | PDN | F | 2 | 2 | | T | 1 | 2 | | No Data | 4 | | | F | 1 | 2 | | T | 2 | 3 | | T | T |
| | PDN | F | 2 | 2 | | T | 1 | 2 | | No Data | 4 | | | F | 1 | 2 | | F | 1 | 3 | | T | T |
| | PDN | F | 2 | 2 | | T | 1 | 2 | | No Data | 4 | | | F | 1 | 2 | | No Data | 4 | | | T | T |
| | N | F | 2 | 2 | | T | 1 | 2 | | No Data | 4 | | | No Data | 4 | | | T | 2 | 3 | | T | Indet. |
| | N | F | 2 | 2 | | T | 1 | 2 | | No Data | 4 | | | No Data | 4 | | | F | 1 | 3 | | T | Indet. |
| | N | F | 2 | 2 | | T | 1 | 2 | | No Data | 4 | | | No Data | 4 | | | No Data | 4 | | | T | Indet. |
| | PDN | F | 2 | 2 | | F | 2 | 2 | | T | 1 | 2 | | T | 2 | 2 | | T | 2 | 3 | | T | T |
| | PDN | F | 2 | 2 | | F | 2 | 2 | | T | 1 | 2 | | T | 2 | 2 | | F | 1 | 3 | | T | T |
| | PDN | F | 2 | 2 | | F | 2 | 2 | | T | 1 | 2 | | T | 2 | 2 | | No Data | 4 | | | T | T |
| | PDN | F | 2 | 2 | | F | 2 | 2 | | T | 1 | 2 | | F | 1 | 2 | | T | 2 | 3 | | T | T |
| | PDN | F | 2 | 2 | | F | 2 | 2 | | T | 1 | 2 | | F | 1 | 2 | | F | 1 | 3 | | T | T |
| | PDN | F | 2 | 2 | | F | 2 | 2 | | T | 1 | 2 | | F | 1 | 2 | | No Data | 4 | | | T | T |
| | PDN | F | 2 | 2 | | F | 2 | 2 | | T | 1 | 2 | | No Data | 4 | | | T | 2 | 3 | | T | T |
| | PDN | F | 2 | 2 | | F | 2 | 2 | | T | 1 | 2 | | No Data | 4 | | | F | 1 | 3 | | T | T |
| | PDN | F | 2 | 2 | | F | 2 | 2 | | T | 1 | 2 | | No Data | 4 | | | No Data | 4 | | | T | T |
| | PDN | F | 3 | 2 | | F | 3 | 2 | | F | 3 | 2 | | T | 3 | 2 | | T | 3 | 3 | | F | F |
| | PDN | F | 3 | 2 | | F | 3 | 2 | | F | 3 | 2 | | T | 3 | 2 | | F | 2 | 3 | | F | F |
| | PDN | F | 3 | 2 | | F | 3 | 2 | | F | 3 | 2 | | T | 3 | 2 | | No Data | 4 | | | F | F |
| | PDN | F | 2 | 2 | | F | 2 | 2 | | F | 2 | 2 | | F | 1 | 2 | | T | 2 | 3 | | T | T |
| | PDN | F | 2 | 2 | | F | 2 | 2 | | F | 2 | 2 | | F | 1 | 2 | | F | 1 | 3 | | T | T |
| | PDN | F | 2 | 2 | | F | 2 | 2 | | F | 2 | 2 | | F | 1 | 2 | | No Data | 4 | | | T | T |
| | PDN | F | 3 | 2 | | F | 3 | 2 | | F | 3 | 2 | | No Data | 4 | | | T | 3 | 3 | | F | Indet. |
| | PDN | F | 3 | 2 | | F | 3 | 2 | | F | 3 | 2 | | No Data | 4 | | | F | 2 | 3 | | F | Indet. |
| | PDN | F | 3 | 2 | | F | 3 | 2 | | F | 3 | 2 | | No Data | 4 | | | No Data | 4 | | | F | Indet. |
| | PDN | F | 3 | 2 | | F | 3 | 2 | | No Data | 4 | | | T | 3 | 2 | | T | 3 | 3 | | F | Indet. |
| | PDN | F | 3 | 2 | | F | 3 | 2 | | No Data | 4 | | | T | 3 | 2 | | F | 2 | 3 | | F | Indet. |
| | PDN | F | 3 | 2 | | F | 3 | 2 | | No Data | 4 | | | T | 3 | 2 | | No Data | 4 | | | F | Indet. |
| | PDN | F | 2 | 2 | | F | 2 | 2 | | No Data | 4 | | | F | 1 | 2 | | T | 2 | 3 | | T | T |
| | PDN | F | 2 | 2 | | F | 2 | 2 | | No Data | 4 | | | F | 1 | 2 | | F | 1 | 3 | | T | T |
| | PDN | F | 2 | 2 | | F | 2 | 2 | | No Data | 4 | | | F | 1 | 2 | | No Data | 4 | | | T | T |
| | N | F | 3 | 2 | | F | 3 | 2 | | No Data | 4 | | | No Data | 4 | | | T | 3 | 3 | | F | Indet. |
| | N | F | 3 | 2 | | F | 3 | 2 | | No Data | 4 | | | No Data | 4 | | | F | 2 | 3 | | F | Indet. |
| | N | F | 3 | 2 | | F | 3 | 2 | | No Data | 4 | | | No Data | 4 | | | No Data | 4 | | | F | Indet. |
| | PDN | F | 2 | 2 | | No Data | 4 | | | T | 1 | 2 | | T | 2 | 2 | | T | 2 | 3 | | T | T |
| | PDN | F | 2 | 2 | | No Data | 4 | | | T | 1 | 2 | | T | 2 | 2 | | F | 1 | 3 | | T | T |
| | PDN | F | 2 | 2 | | No Data | 4 | | | T | 1 | 2 | | T | 2 | 2 | | No Data | 4 | | | T | T |
| | PDN | F | 2 | 2 | | No Data | 4 | | | T | 1 | 2 | | F | 1 | 2 | | T | 2 | 3 | | T | T |
| | PDN | F | 2 | 2 | | No Data | 4 | | | T | 1 | 2 | | F | 1 | 2 | | F | 1 | 3 | | T | T |
| | PDN | F | 2 | 2 | | No Data | 4 | | | T | 1 | 2 | | F | 1 | 2 | | No Data | 4 | | | T | T |
| | PDN | F | 2 | 2 | | No Data | 4 | | | T | 1 | 2 | | No Data | 4 | | | T | 2 | 3 | | T | T |
| | PDN | F | 2 | 2 | | No Data | 4 | | | T | 1 | 2 | | No Data | 4 | | | F | 1 | 3 | | T | T |
| | PDN | F | 2 | 2 | | No Data | 4 | | | T | 1 | 2 | | No Data | 4 | | | No Data | 4 | | | T | T |
| | PDN | F | 3 | 2 | | No Data | 4 | | | F | 3 | 2 | | T | 3 | 2 | | T | 3 | 3 | | F | Indet. |
| | PDN | F | 3 | 2 | | No Data | 4 | | | F | 3 | 2 | | T | 3 | 2 | | F | 2 | 3 | | F | Indet. |
| | PDN | F | 3 | 2 | | No Data | 4 | | | F | 3 | 2 | | T | 3 | 2 | | No Data | 4 | | | F | Indet. |
| | PDN | F | 2 | 2 | | No Data | 4 | | | F | 2 | 2 | | F | 1 | 2 | | T | 2 | 3 | | T | T |
| | PDN | F | 2 | 2 | | No Data | 4 | | | F | 2 | 2 | | F | 1 | 2 | | F | 1 | 3 | | T | T |
| | PDN | F | 2 | 2 | | No Data | 4 | | | F | 2 | 2 | | F | 1 | 2 | | No Data | 4 | | | T | T |
| | PDN | F | 3 | 2 | | No Data | 4 | | | F | 3 | 2 | | No Data | 4 | | | T | 3 | 3 | | F | Indet. |
| | PDN | F | 3 | 2 | | No Data | 4 | | | F | 3 | 2 | | No Data | 4 | | | F | 2 | 3 | | F | Indet. |
| | PDN | F | 3 | 2 | | No Data | 4 | | | F | 3 | 2 | | No Data | 4 | | | No Data | 4 | | | F | Indet. |
| | PDN | F | 3 | 2 | | No Data | 4 | | | No Data | 4 | | | T | 3 | 2 | | T | 3 | 3 | | F | Indet. |
| | PDN | F | 3 | 2 | | No Data | 4 | | | No Data | 4 | | | T | 3 | 2 | | F | 2 | 3 | | F | Indet. |
| | PDN | F | 3 | 2 | | No Data | 4 | | | No Data | 4 | | | T | 3 | 2 | | No Data | 4 | | | F | Indet. |
| | PDN | F | 2 | 2 | | No Data | 4 | | | No Data | 4 | | | F | 1 | 2 | | T | 2 | 3 | | T | T |
| | PDN | F | 2 | 2 | | No Data | 4 | | | No Data | 4 | | | F | 1 | 2 | | F | 1 | 3 | | T | T |
| | PDN | F | 2 | 2 | | No Data | 4 | | | No Data | 4 | | | F | 1 | 2 | | No Data | 4 | | | T | T |
| | N | F | 3 | 2 | | No Data | 4 | | | No Data | 4 | | | No Data | 4 | | | T | 3 | 3 | | F | Indet. |
| | N | F | 3 | 2 | | No Data | 4 | | | No Data | 4 | | | No Data | 4 | | | F | 2 | 3 | | F | Indet. |
| | N | F | 3 | 2 | | No Data | 4 | | | No Data | 4 | | | No Data | 4 | | | No Data | 4 | | | F | Indet. |
| | PDN | No Data | 4 | | | T | 1 | 2 | | T | 1 | 2 | | T | 2 | 2 | | T | 2 | 3 | | T | T |
| | PDN | No Data | 4 | | | T | 1 | 2 | | T | 1 | 2 | | T | 2 | 2 | | F | 1 | 3 | | T | T |
| | PDN | No Data | 4 | | | T | 1 | 2 | | T | 1 | 2 | | T | 2 | 2 | | No Data | 4 | | | T | T |
| | PDN | No Data | 4 | | | T | 1 | 2 | | T | 1 | 2 | | F | 1 | 2 | | T | 2 | 3 | | T | T |
| | PDN | No Data | 4 | | | T | 1 | 2 | | T | 1 | 2 | | F | 1 | 2 | | F | 1 | 3 | | T | T |
| | PDN | No Data | 4 | | | T | 1 | 2 | | T | 1 | 2 | | F | 1 | 2 | | No Data | 4 | | | T | T |
| | PDN | No Data | 4 | | | T | 1 | 2 | | T | 1 | 2 | | No Data | 4 | | | T | 2 | 3 | | T | T |
| | PDN | No Data | 4 | | | T | 1 | 2 | | T | 1 | 2 | | No Data | 4 | | | F | 1 | 3 | | T | T |
| | PDN | No Data | 4 | | | T | 1 | 2 | | T | 1 | 2 | | No Data | 4 | | | No Data | 4 | | | T | T |
| | N | No Data | 4 | | | T | 1 | 2 | | F | 2 | 2 | | T | 2 | 2 | | T | 2 | 3 | | T | T |
| | N | No Data | 4 | | | T | 1 | 2 | | F | 2 | 2 | | T | 2 | 2 | | F | 1 | 3 | | T | T |
| | N | No Data | 4 | | | T | 1 | 2 | | F | 2 | 2 | | T | 2 | 2 | | No Data | 4 | | | T | T |
| | PDN | No Data | 4 | | | T | 1 | 2 | | F | 2 | 2 | | F | 1 | 2 | | T | 2 | 3 | | T | T |
| | PDN | No Data | 4 | | | T | 1 | 2 | | F | 2 | 2 | | F | 1 | 2 | | F | 1 | 3 | | T | T |
| | PDN | No Data | 4 | | | T | 1 | 2 | | F | 2 | 2 | | F | 1 | 2 | | No Data | 4 | | | T | T |
| | N | No Data | 4 | | | T | 1 | 2 | | F | 2 | 2 | | No Data | 4 | | | T | 2 | 3 | | T | Indet. |
| | N | No Data | 4 | | | T | 1 | 2 | | F | 2 | 2 | | No Data | 4 | | | F | 1 | 3 | | T | Indet. |
| | N | No Data | 4 | | | T | 1 | 2 | | F | 2 | 2 | | No Data | 4 | | | No Data | 4 | | | T | Indet. |
| | N | No Data | 4 | | | T | 1 | 2 | | No Data | 4 | | | T | 2 | 2 | | T | 2 | 3 | | T | Indet. |
| | N | No Data | 4 | | | T | 1 | 2 | | No Data | 4 | | | T | 2 | 2 | | F | 1 | 3 | | T | Indet. |
| | N | No Data | 4 | | | T | 1 | 2 | | No Data | 4 | | | T | 2 | 2 | | No Data | 4 | | | T | Indet. |
| | PDN | No Data | 4 | | | T | 1 | 2 | | No Data | 4 | | | F | 1 | 2 | | T | 2 | 3 | | T | T |
| | PDN | No Data | 4 | | | T | 1 | 2 | | No Data | 4 | | | F | 1 | 2 | | F | 1 | 3 | | T | T |
| | PDN | No Data | 4 | | | T | 1 | 2 | | No Data | 4 | | | F | 1 | 2 | | No Data | 4 | | | T | T |
| | N | No Data | 4 | | | T | 1 | 2 | | No Data | 4 | | | No Data | 4 | | | T | 2 | 3 | | T | Indet. |
| | N | No Data | 4 | | | T | 1 | 2 | | No Data | 4 | | | No Data | 4 | | | F | 1 | 3 | | T | Indet. |
| | N | No Data | 4 | | | T | 1 | 2 | | No Data | 4 | | | No Data | 4 | | | No Data | 4 | | | T | Indet. |
| | PDN | No Data | 4 | | | F | 2 | 2 | | T | 1 | 2 | | T | 2 | 2 | | T | 2 | 3 | | T | T |
| | PDN | No Data | 4 | | | F | 2 | 2 | | T | 1 | 2 | | T | 2 | 2 | | F | 1 | 3 | | T | T |
| | PDN | No Data | 4 | | | F | 2 | 2 | | T | 1 | 2 | | T | 2 | 2 | | No Data | 4 | | | T | T |
| | PDN | No Data | 4 | | | F | 2 | 2 | | T | 1 | 2 | | F | 1 | 2 | | T | 2 | 3 | | T | T |
| | PDN | No Data | 4 | | | F | 2 | 2 | | T | 1 | 2 | | F | 1 | 2 | | F | 1 | 3 | | T | T |
| | PDN | No Data | 4 | | | F | 2 | 2 | | T | 1 | 2 | | F | 1 | 2 | | No Data | 4 | | | T | T |
| | PDN | No Data | 4 | | | F | 2 | 2 | | T | 1 | 2 | | No Data | 4 | | | T | 2 | 3 | | T | T |
| | PDN | No Data | 4 | | | F | 2 | 2 | | T | 1 | 2 | | No Data | 4 | | | F | 1 | 3 | | T | T |
| | PDN | No Data | 4 | | | F | 2 | 2 | | T | 1 | 2 | | No Data | 4 | | | No Data | 4 | | | T | T |
| | PDN | No Data | 4 | | | F | 3 | 2 | | F | 3 | 2 | | T | 3 | 2 | | T | 3 | 3 | | F | Indet. |
| | PDN | No Data | 4 | | | F | 3 | 2 | | F | 3 | 2 | | T | 3 | 2 | | F | 2 | 3 | | F | Indet. |
| | PDN | No Data | 4 | | | F | 3 | 2 | | F | 3 | 2 | | T | 3 | 2 | | No Data | 4 | | | F | Indet. |
| | PDN | No Data | 4 | | | F | 2 | 2 | | F | 2 | 2 | | F | 1 | 2 | | T | 2 | 3 | | T | T |
| | PDN | No Data | 4 | | | F | 2 | 2 | | F | 2 | 2 | | F | 1 | 2 | | F | 1 | 3 | | T | T |
| | PDN | No Data | 4 | | | F | 2 | 2 | | F | 2 | 2 | | F | 1 | 2 | | No Data | 4 | | | T | T |
| | PDN | No Data | 4 | | | F | 3 | 2 | | F | 3 | 2 | | No Data | 4 | | | T | 3 | 3 | | F | Indet. |
| | PDN | No Data | 4 | | | F | 3 | 2 | | F | 3 | 2 | | No Data | 4 | | | F | 2 | 3 | | F | Indet. |
| | PDN | No Data | 4 | | | F | 3 | 2 | | F | 3 | 2 | | No Data | 4 | | | No Data | 4 | | | F | Indet. |
| | PDN | No Data | 4 | | | F | 3 | 2 | | No Data | 4 | | | T | 3 | 2 | | T | 3 | 3 | | F | Indet. |
| | PDN | No Data | 4 | | | F | 3 | 2 | | No Data | 4 | | | T | 3 | 2 | | F | 2 | 3 | | F | Indet. |
| | PDN | No Data | 4 | | | F | 3 | 2 | | No Data | 4 | | | T | 3 | 2 | | No Data | 4 | | | F | Indet. |
| | PDN | No Data | 4 | | | F | 2 | 2 | | No Data | 4 | | | F | 1 | 2 | | T | 2 | 3 | | T | T |
| | PDN | No Data | 4 | | | F | 2 | 2 | | No Data | 4 | | | F | 1 | 2 | | F | 1 | 3 | | T | T |
| | PDN | No Data | 4 | | | F | 2 | 2 | | No Data | 4 | | | F | 1 | 2 | | No Data | 4 | | | T | T |
| | N | No Data | 4 | | | F | 3 | 2 | | No Data | 4 | | | No Data | 4 | | | T | 3 | 3 | | F | Indet. |
| | N | No Data | 4 | | | F | 3 | 2 | | No Data | 4 | | | No Data | 4 | | | F | 2 | 3 | | F | Indet. |
| | N | No Data | 4 | | | F | 3 | 2 | | No Data | 4 | | | No Data | 4 | | | No Data | 4 | | | F | Indet. |
| | PDN | No Data | 4 | | | No Data | 4 | | | T | 1 | 2 | | T | 2 | 2 | | T | 2 | 3 | | T | T |
| | PDN | No Data | 4 | | | No Data | 4 | | | T | 1 | 2 | | T | 2 | 2 | | F | 1 | 3 | | T | T |
| | PDN | No Data | 4 | | | No Data | 4 | | | T | 1 | 2 | | T | 2 | 2 | | No Data | 4 | | | T | T |
| | PDN | No Data | 4 | | | No Data | 4 | | | T | 1 | 2 | | F | 1 | 2 | | T | 2 | 3 | | T | T |
| | PDN | No Data | 4 | | | No Data | 4 | | | T | 1 | 2 | | F | 1 | 2 | | F | 1 | 3 | | T | T |
| | PDN | No Data | 4 | | | No Data | 4 | | | T | 1 | 2 | | F | 1 | 2 | | No Data | 4 | | | T | T |
| | PDN | No Data | 4 | | | No Data | 4 | | | T | 1 | 2 | | No Data | 4 | | | T | 2 | 3 | | T | T |
| | PDN | No Data | 4 | | | No Data | 4 | | | T | 1 | 2 | | No Data | 4 | | | F | 1 | 3 | | T | T |
| | PDN | No Data | 4 | | | No Data | 4 | | | T | 1 | 2 | | No Data | 4 | | | No Data | 4 | | | T | T |
| | PDN | No Data | 4 | | | No Data | 4 | | | F | 3 | 2 | | T | 3 | 2 | | T | 3 | 3 | | F | Indet. |
| | PDN | No Data | 4 | | | No Data | 4 | | | F | 3 | 2 | | T | 3 | 2 | | F | 2 | 3 | | F | Indet. |
| | PDN | No Data | 4 | | | No Data | 4 | | | F | 3 | 2 | | T | 3 | 2 | | No Data | 4 | | | F | Indet. |
| | PDN | No Data | 4 | | | No Data | 4 | | | F | 2 | 2 | | F | 1 | 2 | | T | 2 | 3 | | T | T |
| | PDN | No Data | 4 | | | No Data | 4 | | | F | 2 | 2 | | F | 1 | 2 | | F | 1 | 3 | | T | T |
| | PDN | No Data | 4 | | | No Data | 4 | | | F | 2 | 2 | | F | 1 | 2 | | No Data | 4 | | | T | T |
| | PDN | No Data | 4 | | | No Data | 4 | | | F | 3 | 2 | | No Data | 4 | | | T | 3 | 3 | | F | Indet. |
| | PDN | No Data | 4 | | | No Data | 4 | | | F | 3 | 2 | | No Data | 4 | | | F | 2 | 3 | | F | Indet. |
| | PDN | No Data | 4 | | | No Data | 4 | | | F | 3 | 2 | | No Data | 4 | | | No Data | 4 | | | F | Indet. |
| | PDN | No Data | 4 | | | No Data | 4 | | | No Data | 4 | | | T | 3 | 2 | | T | 3 | 3 | | F | Indet. |
| | PDN | No Data | 4 | | | No Data | 4 | | | No Data | 4 | | | T | 3 | 2 | | F | 2 | 3 | | F | Indet. |
| | PDN | No Data | 4 | | | No Data | 4 | | | No Data | 4 | | | T | 3 | 2 | | No Data | 4 | | | F | Indet. |
| | PDN | No Data | 4 | | | No Data | 4 | | | No Data | 4 | | | F | 1 | 2 | | T | 2 | 3 | | T | T |
| | PDN | No Data | 4 | | | No Data | 4 | | | No Data | 4 | | | F | 1 | 2 | | F | 1 | 3 | | T | T |
| | PDN | No Data | 4 | | | No Data | 4 | | | No Data | 4 | | | F | 1 | 2 | | No Data | 4 | | | T | T |
| | PDN | No Data | 4 | | | No Data | 4 | | | No Data | 4 | | | No Data | 4 | | | T | 3 | 3 | | Indet. | Indet. |
| | PDN | No Data | 4 | | | No Data | 4 | | | No Data | 4 | | | No Data | 4 | | | F | 1 | 3 | | Indet. | Indet. |
| | PDN | No Data | 4 | | | No Data | 4 | | | No Data | 4 | | | No Data | 4 | | | No Data | 4 | | | Indet. | Indet. |

Table 23 contains the references used to predict benefit level and provide an evidence level as shown in Table 22 above. The “Ref. No.” column in Table 23 corresponds to the “Ref. No.” columns in Table 22. Specifically, the reference numbers in Table 22 include those references indicated in Table 23.

TABLE 23
References for Comprehensive Cancer Molecular Profile

| Ref. | |
|---|---|
| No. | References |
| 1 | Gong, W., J. Dong, et. al. (2012). “RRM1 expression and clinical outcome of gemcitabine- |
| | containing chemotherapy for advanced non-small-cell lung cancer: A meta-analysis.” Lung |
| | Cancer. 75: 374-380. |
| 2 | Qiu, L. X., M. H. Zheng, et. al. (2008). “Predictive value of thymidylate synthase expression in |
| | advanced colorectal cancer patients receiving fluoropyrimidine-based chemotherapy: Evidence |
| | from 24 studies.” Int. J. Cancer: 123, 2384-2389. |
| | Chen, C.-Y., P.-C. Yang, et al. (2011). “Thymidylate synthase and dihydrofolate reductase |
| | expression in non-small cell lung carcinoma: The association with treatment efficacy of |
| | pemetrexed.” Lung Cancer 74(1): 132-138. |
| | Lee, S. J., Y. H. Im, et. al. (2010). “Thymidylate synthase and thymidine phosphorylase as |
| | predictive markers of capecitabine monotherapy in patients with anthracycline- and taxane- |
| | pretreated metastatic breast cancer.” Cancer Chemother. Pharmacol. DOI 10.1007/s00280- |
| | 010-1545-0. |
| 3 | Braun, M. S., M. T. Seymour, et. al. (2008). “Predictive biomarkers of chemotherapy efficacy in |
| | colorectal cancer: results from the UK MRC FOCUS trial.” J. Clin. Oncol. 26: 2690-2698. |
| | Kostopoulos, I., G. Fountzilas, et. al. (2009). “Topoisomerase I but not thymidylate synthase is |
| | associated with improved outcome in patients with resected colorectal cancer treated with |
| | irinotecan containing adjuvant chemotherapy.” BMC Cancer. 9: 339. |
| | Ataka, M., K. Katano, et. al. (2007). “Topoisomerase I protein expression and prognosis of |
| | patients with colorectal cancer.” Yonago Acta medica. 50: 81-87. |
| 4 | Chinot, O. L., M. Barrie, et al. (2007). “Correlation between O6-methylguanine-DNA |
| | methyltransferase and survival in inoperable newly diagnosed glioblastoma patients treated |
| | with neoadjuvant temozolomide.” J Clin Oncol 25(12): 1470-5. |
| | Kulke, M. H., M. S. Redston, et al. (2008). “06-Methylguanine DNA Methyltransferase |
| | Deficiency and Response to Temozolomide-Based Therapy in Patients with Neuroendocrine |
| | Tumors.” Clin Cancer Res 15(1): 338-345. |
| 5 | El Sheikh, S. S., H. M. Romanska, et. al. (2008). “Predictive value of PTEN and AR |
| | coexpression of sustained responsiveness to hormonal therapy in prostate cancer--a pilot |
| | study.” Neoplasia. 10(9): 949-53. |
| 6 | Lewis, J. D., M. J. Edwards, et al. (2010). “Excellent outcomes with adjuvant toremifene or |
| | tamoxifen in early stage breast cancer.” Cancer116: 2307-15. |
| | Bartlett, J. M. S., D. Rea, et al. (2011). “Estrogen receptor and progesterone receptor as |
| | predictive biomarkers of response to endocrine therapy: a prospectively powered pathology |
| | study in the Tamoxifen and Exemestane Adjuvant Multinational trial.” J Clin Oncol 29 |
| | (12): 1531-1538. |
| | Dowsett, M., C. Allred, et al. (2008). “Relationship between quantitative estrogen and |
| | progesterone receptor expression and human epidermal growth factor receptor 2 (HER-2) |
| | status with recurrence in the Arimidex, Tamoxifen, Alone or in Combination trial.” J Clin |
| | Oncol 26(7): 1059-65. |
| | Viale, G., M. M. Regan, et al. (2008). “Chemoendocrine compared with endocrine adjuvant |
| | therapies for node-negative breast cancer: predictive value of centrally reviewed expression of |
| | estrogen and progesterone receptors--International Breast Cancer Study Group.” J Clin Oncol |
| | 26(9): 1404-10. |
| | Anderson, H., M. Dowsett, et. al. (2011). “Relationship between estrogen receptor, |
| | progesterone receptor, HER-2 and Ki67 expression and efficacy of aromatase inhibitors in |
| | advanced breast cancer. Annals of Oncology. 22: 1770-1776. |
| | Coombes, R. C., J. M. Bliss, et al. (2007). “Survival and safety of exemestane versus tamoxifen |
| | after 2-3 years' tamoxifen treatment (Intergroup Exemestane Study): a randomized controlled |
| | trial.” The Lancet 369: 559-570. |
| | Stuart, N. S. A., H. Earl, et. al. (1996). “A randomized phase III cross-over study of tamoxifen |
| | versus megestrol acetate in advanced and recurrent breast cancer.” European Journal of |
| | Cancer. 32(11): 1888-1892. |
| | Thurlimann, B., A. Goldhirsch, et al. (1997). “Formestane versus Megestrol Acetate in |
| | Postmenopausal Breast Cancer Patients After Failure of Tamoxifen: A Phase III Prospective |
| | Randomised Cross Over Trial of Second-line Hormonal Treatment (SAKK 20/90). E J Cancer |
| | 33 (7): 1017-1024. |
| | Cuzick J, LHRH-agonists in Early Breast Cancer Overview group. (2007). “Use of luteinising- |
| | hormone-releasing hormone agonists as adjuvant treatment in premenopausal patients with |
| | hormone-receptor-positive breast cancer: a meta-analysis of individual patient data from |
| | randomised adjuvant trials.” The Lancet 369: 1711-1723. |
| 7 | Lewis, J. D., M. J. Edwards, et al. (2010). “Excellent outcomes with adjuvant toremifene or |
| | tamoxifen in early stage breast cancer.” Cancer116: 2307-15. |
| | Stendahl, M., L. Ryden, et al. (2006). “High progesterone receptor expression correlates to the |
| | effect of adjuvant tamoxifen in premenopausal breast cancer patients.” Clin Cancer Res |
| | 12(15): 4614-8. |
| | Bartlett, J. M. S., D. Rea, et al. (2011). “Estrogen receptor and progesterone receptor as |
| | predictive biomarkers of response to endocrine therapy: a prospectively powered pathology |
| | study in the Tamoxifen and Exemestane Adjuvant Multinational trial.” J Clin Oncol 29 |
| | (12): 1531-1538. |
| | Dowsett, M., C. Allred, et al. (2008). “Relationship between quantitative estrogen and |
| | progesterone receptor expression and human epidermal growth factor receptor 2 (HER-2) |
| | status with recurrence in the Arimidex, Tamoxifen, Alone or in Combination trial.” J Clin |
| | Oncol 26(7): 1059-65. |
| | Coombes, R. C., J. M. Bliss, et al. (2007). “Survival and safety of exemestane versus tamoxifen |
| | after 2-3 years' tamoxifen treatment (Intergroup Exemestane Study): a randomized controlled |
| | trial.” The Lancet 369: 559-570. |
| | Yamashita, H., Y. Yando, et al. (2006). “Immunohistochemical evaluation of hormone |
| | receptor status for predicting response to endocrine therapy in metastatic breast cancer.” Breast |
| | Cancer 13(1): 74-83. |
| | Stuart, N. S. A., H. Earl, et. al. (1996). “A randomized phase III cross-over study of tamoxifen |
| | versus megestrol acetate in advanced and recurrent breast cancer.” European Journal of |
| | Cancer. 32(11): 1888-1892. |
| | Thurlimann, B., A. Goldhirsch, et al. (1997). “Formestane versus Megestrol Acetate in |
| | Postmenopausal Breast Cancer Patients After Failure of Tamoxifen: A Phase III Prospective |
| | Randomised Cross Over Trial of Second-line Hormonal Treatment (SAKK 20/90). E J Cancer |
| | 33 (7): 1017-1024. |
| | Cuzick J, LHRH-agonists in Early Breast Cancer Overview group. (2007). “Use of luteinising- |
| | hormone-releasing hormone agonists as adjuvant treatment in premenopausal patients with |
| | hormone-receptor-positive breast cancer: a meta-analysis of individual patient data from |
| | randomised adjuvant trials.” The Lancet 369: 1711-1723. |
| 8 | Amir, E. et. al. (2010). “Lapatinib and HER2 status: results of a meta-analysis of randomized |
| | phase III trials in metastatic breast cancer.” Cancer Treatment Reviews. 36: 410-415. |
| | Johnston, S., Pegram M., et. al. (2009). “Lapatinib combined with letrozole versus letrozole |
| | and placebo as first-line therapy for postmenopausal hormone receptor-positive metastatic |
| | breast cancer. Journal of Clinical Oncology. Published ahead of print on Sep. 28, 2009 |
| | as 10.1200/JCO.2009.23.3734. |
| | Press, M. F., R. S. Finn, et al. (2008). “HER-2 gene amplification, HER-2 and epidermal |
| | growth factor receptor mRNA and protein expression, and lapatinib efficacy in women with |
| | metastatic breast cancer.” Clin Cancer Res 14(23): 7861-70. |
| 9 | Amir, E. et. al. (2010). “Lapatinib and HER2 status: results of a meta-analysis of randomized |
| | phase III trials in metastatic breast cancer.” Cancer Treatment Reviews. 36: 410-415. |
| | Johnston, S., Pegram M., et. al. (2009). “Lapatinib combined with letrozole versus letrozole |
| | and placebo as first-line therapy for postmenopausal hormone receptor-positive metastatic |
| | breast cancer. Journal of Clinical Oncology. Published ahead of print on Sep. 28, 2009 |
| | as 10.1200/JCO.2009.23.3734. |
| | Press, M. F., R. S. Finn, et al. (2008). “HER-2 gene amplification, HER-2 and epidermal |
| | growth factor receptor mRNA and protein expression, and lapatinib efficacy in women with |
| | metastatic breast cancer.” Clin Cancer Res 14(23): 7861-70. |
| | Bartlett, J. M. S., K. Miller, et. al. (2011). “A UK NEQAS ISH multicenter ring study using the |
| | Ventana HER2 dual-color ISH assay.” Am. J. Clin. Pathol. 135: 157-162. |
| 10 | Slamon, D., M. Buyse, et. al. (2011). “Adjuvant trastuzumab in HER2-positive breast cancer.” |
| | N. Engl. J. Med. 365: 1273-83. |
| | Yin, W., J. Lu, et. al. (2011). “Trastuzumab in adjuvant treatment HER2-positive early breast |
| | cancer patients: A meta-analysis of published randomized controlled trials.” PLoS ONE 6(6): |
| | e21030. doi: 10.1371/journal.pone.0021030. |
| | Cortes, J., J. Baselga, et. al. (2012). “Pertuzumab monotherapy after trastuzumab-based |
| | treatment and subsequent reintroduction of trastuzumab: activity and tolerability in patients |
| | with advanced human epidermal growth factor receptor-2-positive breast cancer.” J. Clin. |
| | Oncol. 30. DOI: 10.1200/JCO.2011.37.4207. |
| | Bang, Y-J., Y-K. Kang, et. al. (2010). “Trastuzumab in combination with chemotherapy versus |
| | chemotherapy alone for treatment of HER2-positive advanced gastric or gastro-oesophageal |
| | junction cancer (ToGA): a phase 3, open-label, randomised controlled trial.” Lancet. 376: |
| | 687-97. |
| | Baselga, J., S. M. Swain, et. al. (2012). “Pertuzumab plus trastuzumab plus docetaxel for |
| | metastatic breast cancer”. N. Engl. J. Med. 36: 109-119. |
| | Verma, S., K. Blackwell, et. al. (2012) “Trastuzumab Emtansine for HER2-Positive Advanced |
| | Breast Cancer” N Engl J Med. 367(19): 1783-91. |
| | Hurvitz, S. A., E. A. Perez, et. al. (2013) “Phase II randomized study of trastuzumab emtansine |
| | versus trastuzumab plus docetaxel in patients with human epidermal growth factor receptor 2- |
| | positive metastatic breast cancer.” J Clin Oncol.31 (9): 1157-63 |
| 11 | Slamon, D., M. Buyse, et. al. (2011). “Adjuvant trastuzumab in HER2-positive breast cancer.” |
| | N. Engl. J. Med. 365: 1273-83. |
| | Yin, W., J. Lu, et. al. (2011). “Trastuzumab in adjuvant treatment HER2-positive early breast |
| | cancer patients: A meta-analysis of published randomized controlled trials.” PLoS ONE 6(6): |
| | e21030. doi: 10.1371/journal.pone.0021030. |
| | Cortes, J., J. Baselga, et. al. (2012). “Pertuzumab monotherapy after trastuzumab-based |
| | treatment and subsequent reintroduction of trastuzumab: activity and tolerability in patients |
| | with advanced human epidermal growth factor receptor-2-positive breast cancer.” J. Clin. |
| | Oncol. 30. DOI: 10.1200/JCO.2011.37.4207. |
| | Bang, Y-J., Y-K. Kang, et. al. (2010). “Trastuzumab in combination with chemotherapy versus |
| | chemotherapy alone for treatment of HER2-positive advanced gastric or gastro-oesophageal |
| | junction cancer (ToGA): a phase 3, open-label, randomised controlled trial.” Lancet. 376: |
| | 687-97. |
| | Bartlett, J. M. S., K. Miller, et. al. (2011). “A UK NEQAS ISH multicenter ring study using the |
| | Ventana HER2 dual-color ISH assay.” Am. J. Clin. Pathol. 135: 157-162. |
| | Baselga, J., S. M. Swain, et. al. (2012). “Pertuzumab plus trastuzumab plus docetaxel for |
| | metastatic breast cancer”. N. Engl. J. Med. 36: 109-119. |
| | Verma, S., K. Blackwell, et. al. (2012) “Trastuzumab Emtansine for HER2-Positive Advanced |
| | Breast Cancer” N Engl J Med. 367(19): 1783-91. |
| | Hurvitz, S. A., E. A. Perez, et. al. (2013) “Phase II randomized study of trastuzumab emtansine |
| | versus trastuzumab plus docetaxel in patients with human epidermal growth factor receptor 2- |
| | positive metastatic breast cancer.” J Clin Oncol.31(9): 1157-63 |
| 12 | Press, M. F., Slamon, D. J., et. al. (2011). “Alteration of topoisomerase II-alpha gene in human |
| | breast cancer: association with responsiveness to anthracycline based chemotherapy.” J. Clin. |
| | Oncol, 29(7): 859-67. |
| | Du, Y., J. Lu, et. al. (2011). “The role of topoisomerase II a in predicting sensitivity to |
| | anthracyclines in breast cancer patients: a meta-analysis of published literatures.” Breast Can |
| | Res Treat. 129(3): 839-848. |
| | O'Malley, F. P., K. I. Pritchard, et. al (2009) “Topoisomerase II alpha and responsiveness of |
| | breast cancer to adjuvant chemotherapy.” J Natl Can Inst. 101: 644-650. |
| | Tanner, M., J. Bergh, et al. (2006). “Topoisomerase II-α Gene Amplification Predicts |
| | Favorable Treatment Response to Tailored and Dose-Escalated Anthracycline-Based Adjuvant |
| | Chemotherapy in HER-2/neu-Amplified Breast Cancer: Scandinavian Breast Group Trial |
| | 9401.” J Clin Oncol 24(16): 2428-2436. |
| 13 | Press, M. F., Slamon, D. J., et. al. (2011). “Alteration of topoisomerase II-alpha gene in human |
| | breast cancer: association with responsiveness to anthracycline based chemotherapy.” J. Clin. |
| | Oncol, 29(7): 859-67. |
| | Gennari, A., P. Bruzzi, et. al (2008) “HERZ status and efficacy of adjuvant anthracyclines in |
| | early breast cancer: a pooled analysis of randomized trials.” J Natl Can Inst. 100: 14-20. |
| 14 | O'Malley, F. P., K. I. Pritchard, et al. (2011). “Topoisomerase II alpha protein and resposiveness |
| | of breast cancer to adjuvant chemotherapy with CEF compared to CMF in the NCIC CTG |
| | randomized MA.5 adjuvant trial.” Breast Can Res Treat. 128, 401-409. |
| | Rodrigo, R. S., C. Axel le, et. al. (2011). “Topoisomerase II-alpha protein expression and |
| | histological response following doxorubicin-based induction chemotherapy predict survival of |
| | locally advanced soft tissues sarcomas.” Eur J of Can. 47, 1319-1327. |
| 15 | Chintamani, J. P., Singh, et. al. (2005). “Role of p-glycoprotein expression in predicting |
| | response to neoadjuvant chemotherapy in breast cancer - a prospective clinical study.” World |
| | J. Surg. Oncol. 3: 61. |
| | Akimoto, M., H, Saisho, et al. (2006). “Relationship between therapeutic efficacy of arterial |
| | infusion chemotherapy and expression of P-glycoprotein and p53 protein in advanced |
| | hepatocellular carcinoma.” World J of Gastroenterol, 12(6), 868-873. |
| 16 | Carvajal, R. D., G. K. Schwartz, et. al. (2011). “KIT as a therapeutic target in metastatic |
| | melanoma.” JAMA. 305(22): 2327-2334. |
| | Guo, Q. Z., Z. J. Wang, et. al. (2010). “High expression of ERCC1 is a poor prognostic factor in |
| | Chinese patients with non-small cell lung cancer receiving cisplatin-based therapy.” Chin. J. |
| | Cancer Res. 22(4): 296-302. |
| 17 | Cassier, P. A., P. Hohenberger, et al. (2012). “Outcome of Patients with Platelet-Derived |
| | Growth Factor Receptor Alpha-Mutated Gastrointestinal Stromal Tumors in the Tyrosine |
| | Kinase Inhibitor Era.” Clin Cancer Res 18: 4458-4464. |
| | Heinrich, M. C., J. A. Fletcher, et. al. (2008). “Correlation of kinase genotype and clinical |
| | outcome in North American Intergroup phase III trial of imatinib mesylate for treatment of |
| | advanced gastrointestinal stromal tumor: CALGB 150105 study by Cancer and Leukemia |
| | Group B and Southwest Oncology Group.” J Clin Oncol. 26(33): 5360-5367. |
| | Debiec-Rychter, M., I. Judson, et al. (2006). “KIT mutations and dose selection for imatinib in |
| | patients with advanced gastrointestinal stromal tumours.” Eur J Cancer 42: 1093-1103. |
| 18 | Kwak, E. L., A. J. Iafrate, et. al. (2010). “Anaplastic lymphoma kinase inhibition in non-small |
| | cell lung cancer.” N. Engl. J. Med. 363: 1693-703. |
| | Lin, E., Modrusan, Z., (2009). Exon array profiling detects EML4-ALK fusion in breast, |
| | colorectal and non-small lung cancers, Mol. Cancer Res. 7(9): 1466-76. |
| 19 | Bergethon, K., A. J. Iafrate, et. al. (2012) “ROS1 Rearrangements Define a Unique Molecular |
| | Class of Lung Cancers.” J. Clin. Oncol. 30(8): 863-70. |
| | Davies, K. D., R. C. Deobele, et. al. (2012) “Identifying and Targeting ROS1 Gene Fusions in |
| | Non-Small Cell Lung Cancer.” Clin. Cancer Res. 18(17): 4570-9. |
| | Shaw, A. T., S. I. Ou, et. al. (2012) “Clinical activity of crizotinib in advanced non-small cell |
| | lung cancer (NSCLC) harboring ROS1 gene rearrangement.” J Clin Oncol 30 (suppl; abstr |
| | 7508). |
| | National Comprehensive Cancer Network. NCCN Clinical Practice Guidelines in Oncology. |
| | Non-Small Cell Lung Cancer 2.2013. 2013; National Comprehensive Cancer Network. |
| 20 | Janku, F., R. Kurzrock, et. al. (2011). “PIK3CA mutations in patients with advanced cancers |
| | treated with PI3K/AKT/mTOR axis inhibitors.” Molecular Cancer Therapeutics. 10(3): |
| | 558-65. |
| | Janku, F., R. Kurzrock, et. al. (2012). “PI3K/Akt/mTOR inhibitors in patients with breast and |
| | gynecologic malignancies harboring PIK3CA mutations.” Journal of Clinical Oncology. DOI: |
| | 10.1200/JC0.2011.36.1196. |
| | Moroney, J. W., R. Kurzrock, et. al. (2011). “A phase I trial of liposomal doxorubicin, |
| | bevacizumab, and temsirolimus in patients with advanced gynecologic and breast |
| | malignancies.” Clin. Cancer Res. 17: 6840-6846. |
| 21 | Wells, S. A., M. J. Schlumberger, et al. (2012). “Vandetanib in Patients with Locally Advanced |
| | or Metastatic Medullary Thyroid Cancer: A Randomized, Double-Blind Phase III Trial.” J Clin |
| | Oncol 30: 134-141. |
| 22 | Desai, N., Soon-Shiong, P., et al. (2009). “SPARC Expression Correlates with Tumor |
| | Response to Albumin-Bound Paclitaxel in Head and Neck Cancer Patients.” Translational |
| | Oncology 2(2): 59-64. |
| | Von Hoff, D. D., M. Hidalgo, et. al. (2011). “Gemcitabine plus nab-paclitaxel is an active |
| | regimen in patients with advanced pancreatic cancer: a phase I/II trial.” J. Clin. Oncol. DOI: |
| | 10.1200/JC0.2011.36.5742. |
| 23 | Kulkami, S. A., D. T. Ross, et. al. (2009). “TLE3 as a candidate biomarker of response to |
| | taxane therapy”. Breast Cancer Research. 11: R17 (doi: 10.1186/bcr2241). |
| 24 | Zhang, H.-L., X.-W. Zhou, et al. (2012). “Association between class III β-tubulin expression |
| | and response to paclitaxel/vinorelbine-based chemotherapy for non-small cell lung cancer: A |
| | meta-analysis.” Lung Cancer 77: 9-15. |
| | Seve, P., C. Dumontet, et al. (2005). “Class III β-tubulin expression in tumor cells predicts |
| | response and outcome in patients with non-small cell lung cancer receiving paclitaxel.” Mol |
| | Cancer Ther 4(12): 2001-2007. |
| | Gao, S., J. Gao, et al. (2012). “Clinical implications of REST and TUBB3 in ovarian cancer |
| | and its relationship to paclitaxel resistance.” Tumor Biol 33: 1759-1765. |
| | Ploussard, G., A. de la Taille, et al. (2010). “Class III β-Tubulin Expression Predicts Prostate |
| | Tumor Aggressiveness and Patient Response to Docetaxel-Based Chemotherapy.” Clin Cancer |
| | Res 70(22): 9253-9264. |
| 25 | Penson, R. T., M. V. Seiden, et al. (2004). “Expression of multidrug resistance-1 protein |
| | inversely correlates with paclitaxel response and survival in ovarian cancer patients: a study in |
| | serial samples.” Gynecologic Oncology 93: 98-106. |
| | Yeh, J. J., A. Kao, et al. (2003). “Predicting Chemotherapy Response to Paclitaxel-Based |
| | Therapy in Advanced Non-Small-Cell Lung Cancer with P-Glycoprotein Expression.” |
| | Respiration 70: 32-35. |

The PLUS profiles described above and shown in the appropriate panels in FIGS. 33A-33Q include additional sequencing as in Table 24.

TABLE 24
PLUS Sequencing panel

ABL1
AKT1
ALK
APC
ATM
BRAF
CDH1
CSF1R
CTNNB1
EGFR
ERBB2 (Her2)
ERBB4
FBXW7
FGFR1
FGFR2
FLT3
GNA11
GNAQ
GNAS
HNF1A
HRAS
IDH1
JAK2
JAK3
KDR (VGFR2)
cKIT
KRAS
cMET
MLH1
MPL
NOTCH1
NPM1
NRAS
PDGFRA
PIK3CA
PTEN
PTPN11
RBI
RET
SMAD4
SMARCB1
SMO
STK11
TP53
VHL

Any of the biomarker assays herein, e.g., as shown in FIGS. 33A-33Q or Tables 7-24 can be performed individually as desired. One of skill will appreciate that any combination of the individual biomarker assays could be performed. For example, a treating physician may choose to order one or more of the following to profile a particular patient's tumor: IHC for 1, 2, 3, 4, 5, 6, 7, 8, 9, 10, 11, 12, 13, 14, 15, 16 or 17 of AR, cMET, EGFR (including H-score for lung cancer such as NSCLC), ER, HER2, MGMT, Pgp, PR, PTEN, RRM1, SPARCm, SPARCp, TLE3, TOPO1, TOP2A, TS, TUBB3; FISH or CISH for 1, 2, 3, 4, or 5 of ALK, cMET, HER2, ROS1, TOP2A; Mutational Analysis of 1, 2, 3 or 4 of BRAF (e.g., Cobas® PCR), IDH2 (e.g., Sanger Sequencing), MGMT-Me (e.g., by PyroSequencing); EGFR (e.g., fragment analysis to detect EGFRvIII); and/or Mutational Analysis (e.g., by Next-Generation Sequencing) of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 of ABL1, AKT1, ALK, APC, ATM, BRAF, CDH1, CSF1R, CTNNB1, EGFR, ERBB2 (HER2), ERBB4, FBXW7, FGFR1, FGFR2, FLT3, GNA11, GNAQ, GNAS, HNF1A, HRAS, IDH1, JAK2, JAK3, KDR (VEGFR2), KIT, KRAS, MET, MLH1, MPL, NOTCH1, NPM1, NRAS, PDGFRA, PIK3CA, PTEN, PTPN11, RB1, RET, SMAD4, SMARCB1, SMO, STK11, TP53, VHL. In some embodiments, a selection of individual tests is made when insufficient tumor sample is available for performing all molecular profiling tests in FIGS. 33A-33Q or Tables 7-24.

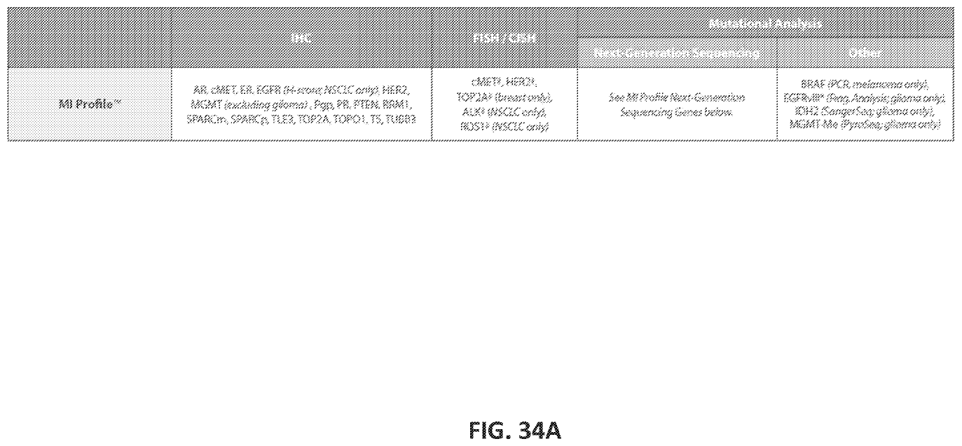

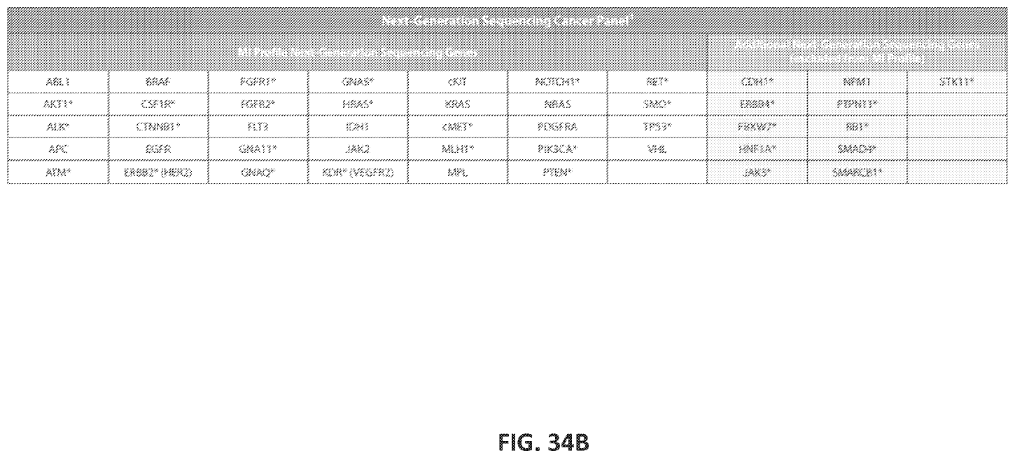

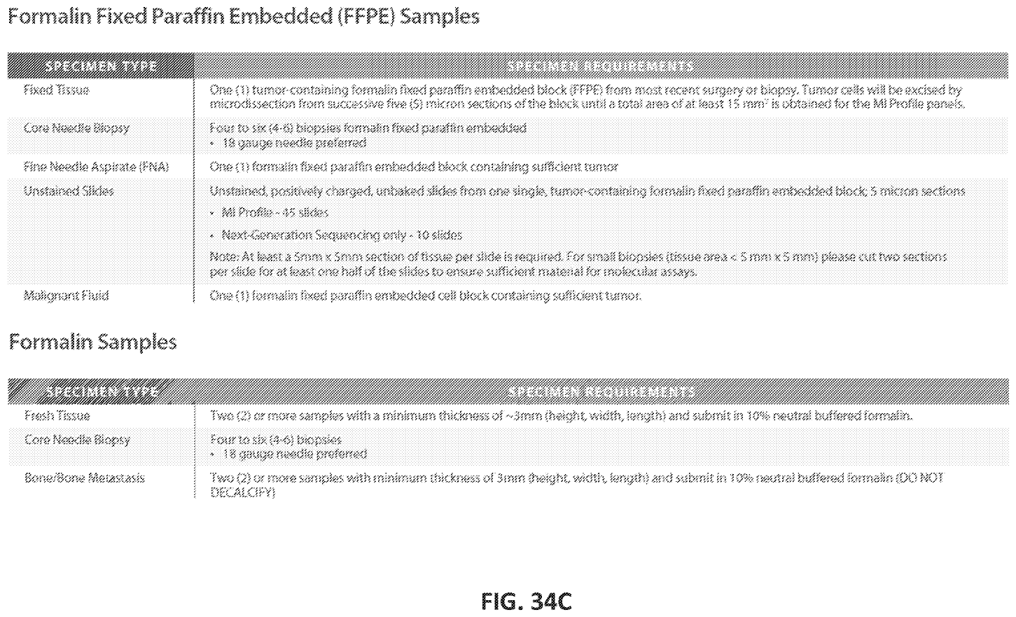

FIGS. 34A-34C illustrate biomarkers assessed using a molecular profiling approach as outlined in FIGS. 33A-33Q or Tables 7-24, and accompanying text herein. FIG. 34A illustrates biomarkers that are assessed. The biomarkers that are assessed according to the Next Generation sequencing panel are shown in FIG. 34B. FIG. 34C illustrates sample requirements that can be used to perform molecular profiling on a patient tumor sample according to the panels in FIGS. 34A-34B.

In certain embodiments, ERCC1 is assessed according to the profiles described below and in FIGS. 33A-Q and Tables 7-24. Lack of ERCC1 expression, e.g., as determined by IHC, can indicate positive benefit for platinum compounds (cisplatin, carboplatin, oxaliplatin), and conversely positive expression of ERCC1 can indicate lack of benefit of these drugs. Additional biomarkers that can be assessed according to the molecular profiles include EGFRvIII, IDH2, and PD1. The presence of EGFRvIII may be assessed using expression analysis at the protein or mRNA level, e.g., by either IHC or PCR, respectively. Expression of EGFRvIII can suggest treatment with EGFR inhibitors. Mutational analysis can be performed for IDH2, e.g., by Sanger sequencing, pyrosequencing or by next generation sequencing approaches. IDH2 mutations suggest the same therapy indications as IDH1 mutations, e.g., for decarbazine and temozolomide as described herein. PD1 (programmed death-1, PD-1) can be assessed at the protein level, e.g., by IHC. Monoclonal antibodies targeting PD-1 that boost the immune system are being developed for the treatment of cancer. See, e.g., Flies et al, Blockade of the B7-H1/PD-1 pathway for cancer immunotherapy. Yale J Biol Med. 2011 December; 84(4):409-21.

### Lab Technique Substitution

One of skill will appreciate that the laboratory techniques of the molecular profiles herein can be substituted by alternative techniques if appropriate, including alternative techniques as disclosed herein or known in the art. For example, FISH and CISH are generally interchangeable methods so that one can often be used in place of the other. Similarly, Dual ISH methods such as described herein can be substituted for conventional ISH methods. In an embodiment, the FDA approved INFORM HER2 Dual ISH DNA Probe Cocktail kit from Ventana Medical Systems, Inc. (Tucson, Ariz.) is used for FISH/CISH analysis of HER2. This kit allows the determination of the HER2 gene status by enumeration of the ratio of the HER2 gene to Chromosome 17. The HER2 and Chromosome 17 probes are detected using two color chromogenic in situ hybridization (CISH) reactions. A number of methods can be used to assess nucleic acid sequences, and any alterations thereof, including without limitation point mutations, insertions, deletions, translocations, rearrangements. Nucleic acid analysis methods include Sanger sequencing, next generation sequencing, polymerase chain reaction (PCR), real-time PCR (qPCR; RT-PCR), a low density microarray, a DNA microarray, a comparative genomic hybridization (CGH) microarray, a single nucleotide polymorphism (SNP) microarray, fragment analysis, RFLP, pyrosequencing, methylation specific PCR, mass spec, Southern blotting, hybridization, and related methods such as described herein. Similarly, a number of methods can be used to assess gene expression, including without limitation next generation sequencing, polymerase chain reaction (PCR), real-time PCR (qPCR; RT-PCR), a low density microarray, a DNA microarray, a comparative genomic hybridization (CGH) microarray, a single nucleotide polymorphism (SNP) microarray, proteomic arrays, antibody arrays or mass spec. The presence or level of a protein can also be assessed using multiple methods as appropriate, including without limitation IHC, immunocapture, immunoblotting, Western analysis, ELISA, immunoprecipitation, flow cytometry, and the like. The desired laboratory technique can be chosen based of multiple criteria, including without limitation accuracy, precision, reproduceability, cost, amount of sample available, type of sample available, time to perform the technique, regulatory approval status of the technique platform, regulatory approval status of the particular test, and the like.

In some embodiments, more than one technique is used to assess a same biomarker. For example, results of profiling both gene expression and protein expression can provide confirmatory results. In other cases, a certain method may provide optimal results depending on the available sample. In some embodiments, sequencing is used to assess EGFR if the sample is more than 50% tumor. Fragment analysis (FA) can also be used to assess EGFR. In some embodiments, FA, e.g., RFLP, is used to assess EGFR if the sample is less than 50% tumor. In still other cases, one technique may indicate a desire to perform another technique, e.g., a less expensive technique or one that requires lesser sample quantity may indicate a desire to perform a more expensive technique or one that consumes more sample. In an embodiment, FA of ALK is performed first, and then FISH or PCR is performed if the FA indicates the presence of a particular ALK alteration such as an ALK fusion. The FISH and/or PCR assay can be designed such that only certain fusion products are detected, e.g., EML4-ALK. The alternate methods may also provide different information about the biomarker. For example, sequence analysis may reveal the presence of a mutant protein, whereas IHC of the protein may reveal its level and/or cellular location. As another example, gene copy number or gene expression at the RNA level may be elevated, but the presence of interfering RNAs may still downregulate protein expression. As still another example, a biomarker can be assessed using a same technique but with different reagents that provide actionable results. As an example, SPARC can be assessed by IHC using either a polyclonal or a monoclonal antibody. This context is identified herein, e.g., as SPARCp, SPARC poly, or variants thereof for SPARC detected using a polyclonal antibody), and as SPARCm, SPARC mono, or variants thereof, for SPARC detected using a monoclonal antibody). SPARC (m/p) and similar derivations can be used to refer to IHC performed using both polyclonal and monoclonal antibodies.

One of skill will appreciate that molecular profiles of the invention can be updated as new evidence becomes available. For example, new evidence may appear in the literature describing an association between a treatment and potential benefit for cancer or a certain lineage of cancer. This information can be incorporated into an appropriate molecular profile. As another example, new evidence may be presented for a biomarker that is already assessed according to the invention. Consider the BRAF V600E mutation that is currently FDA approved for directed treatment with vemurafenib for melanoma. If the treatment is determined to be effective in another setting, e.g., for another lineage of cancer, BRAF V600E can be added to an appropriate molecular profile for that setting.

### Mutational Analysis (4.4+, 4.5, 4.6, 4.7, 5.0)

Mutational or sequence analysis can be performed using any number of techniques described herein or known in the art, including without limitation sequencing (e.g., Sanger, Next Generation, pyrosequencing), PCR, variants of PCR such as RT-PCR, fragment analysis, and the like. Table 25 describes a number of genes bearing mutations that have been identified in various cancer lineages. In an aspect, the invention provides a molecular profile comprising one or more genes in Table 25. In one embodiment, the genes are assessed using Next Generation sequencing methods, e.g., using a TruSeq system offered by Illumina Corporation or an Ion Torrent system from Life Technologies. One of skill will appreciate that the profiling may be used to identify candidate treatments for cancer lineages other than those described in Table 25. Clinical trials in the table can be found at www.clinicaltrials.gov using the indicated identifiers.

TABLE 25
Exemplary Mutated Genes and Gene Products and Related Therapies

| Biomarker | Description |
|---|---|
| ABL1 | Most CML patients have a chromosomal abnormality due to a fusion between |
| | Abelson (Abl) tyrosine kinase gene at chromosome 9 and break point cluster (Bcr) |
| | gene at chromosome 22 resulting in constitutive activation of the Bcr-Abl fusion |
| | gene. Imatinib is a Bcr-Abl tyrosine kinase inhibitor commonly used in treating |
| | CML patients. Mutations in the ABL1 gene are common in imatinib resistant CML |
| | patients which occur in 30-90% of the patients. However, more than 50 different |
| | point mutations in the ABL1 kinase domain may be inhibited by the second |
| | generation kinase inhibitors, dasatinib, bosutinib and nilotinib. The gatekeeper |
| | mutation, T315I that causes resistance to all currently approved TKIs accounts for |
| | about 15% of the mutations found in patients with imatinib resistance. BCR-ABL1 |
| | mutation analysis is recommended to help facilitate selection of appropriate |
| | therapy for patients with CML after treatment with imatinib fails. Agents that |
| | target this biomarker are in clinical trials, e.g.: NCT01528085. |
| AKT1 | AKT1 gene (v-akt murine thymoma viral oncogene homologue 1) encodes a |
| | serine/threonine kinase which is a pivotal mediator of the PI3K-related signaling |
| | pathway, affecting cell survival, proliferation and invasion. Dysregulated AKT |
| | activity is a frequent genetic defect implicated in tumorigenesis and has been |
| | indicated to be detrimental to hematopoiesis. Activating mutation E17K has been |
| | described in breast (2-4%), endometrial (2-4%), bladder cancers (3%), NSCLC |
| | (1%), squamous cell carcinoma of the lung (5%) and ovarian cancer (2%). This |
| | mutation in the pleckstrin homology domain facilitates the recruitment of AKT to |
| | the plasma membrane and subsequent activation by altering phosphoinositide |
| | binding. A mosaic activating mutation E17K has also been suggested to be the |
| | cause of Proteus syndrome. Mutation E49K has been found in bladder cancer, |
| | which enhances AKT activation and shows transforming activity in cell lines. |
| | Agents targeting AKT1 are in clinical trials, e.g., the AKT inhibitor MK-2206 is in |
| | trials for patients carrying AKT mutations (see NCT01277757, NCT01425879). |
| ALK | APC, or adenomatous polyposis coli, is a key tumor suppressor gene that encodes |
| | for a large multi-domain protein. This protein exerts its tumor suppressor function |
| | in the Wnt/β-catcnin cascade mainly by controlling the degradation of β-catenin, |
| | the central activator of transcription in the Wnt signaling pathway. The Wnt |
| | signaling pathway mediates important cellular functions including intercellular |
| | adhesion, stabilization of the cytoskeleton, and cell cycle regulation and apoptosis, |
| | and it is important in embryonic development and oncogenesis. Mutation in APC |
| | results in a truncated protein product with abnormal function, lacking the domains |
| | involved in β-catenin degradation. Somatic mutation in the APC gene can be |
| | detected in the majority of colorectal tumors (80%) and it is an early event in |
| | colorectal tumorigenesis. APC wild type patients have shown better disease control |
| | rate in the metastatic setting when treated with oxaliplatin, while when treated with |
| | fluoropyrimidine regimens, APC wild type patients experience more hematological |
| | toxicities. APC mutation has also been identified in oral squamous cell carcinoma, |
| | gastric cancer as well as hepatoblastoma and may contribute to cancer formation. |
| | Agents that target this gene and/or its downstream or upstream effectors are in |
| | clinical trials, e.g.: NCT01198743. |
| | In addition, germline mutation in APC causes familial adenomatous polyposis, |
| | which is an autosomal dominant inherited disease that will inevitably develop to |
| | colorectal cancer if left untreated. COX-2 inhibitors including celecoxib may |
| | reduce the recurrence of adenomas and incidence of advanced adenomas in |
| | individuals with an increased risk of CRC. Turcot syndrome and Gardner's |
| | syndrome have also been associated with germline APC defects. Germline |
| | mutations of the APC have also been associated with an increased risk of |
| | developing desmoid disease, papillary thyroid carcinoma and hepatoblastoma. |
| APC | APC, or adenomatous polyposis coli, is a key tumor suppressor gene that encodes |
| | for a large multi-domain protein. This protein exerts its tumor suppressor function |
| | in the Wnt/β-catcnin cascade mainly by controlling the degradation of β-catenin, |
| | the central activator of transcription in the Wnt signaling pathway. Wnt signaling |
| | pathway mediates important cellular functions including intercellular adhesion, |
| | stabilization of the cytoskeleton and cell cycle regulation and apoptosis, and is |
| | important in embryonic development and oncogenesis. Mutation in APC results in |
| | a truncated protein product with abnormal function, lacking the domains involved |
| | in β -catenin degradation. Germline mutation is APC causes familial adenomatous |
| | polyposis, which is an autosomal dominant inherited disease that will inevitably |
| | develop to colorectal cancer if left untreated. Somatic mutation in APC gene can be |
| | detected in the majority of colorectal tumors (~80%) and is an early event in |
| | colorectal tumorigenesis. APC mutation has been identified in about 12.5% of oral |
| | squamous cell carcinoma and may contribute to the genesis of the cancer. |
| | Chemoprevention studies in preclinical models show APC deficient pre-malignant |
| | cells respond to a combination of TRAIL (tumor necrosis factor-related apoptosis- |
| | inducing ligand, or Apo2L) and RAc (9-cis-retinyl acetate) in vitro without normal |
| | cells being affected. |
| ATM | ATM, or ataxia telangiectasia mutated, is activated by DNA double-strand breaks |
| | and DNA replication stress. It encodes a protein kinase that acts as a tumor |
| | suppressor and regulates various biomarkers involved in DNA repair, e.g., p53, |
| | BRCA1, CHK2, RAD17, RAD9, and NBS1. ATM is associated with hematologic |
| | malignancies, and somatic mutations have also been found in colon (18.2%), head |
| | and neck (14.3%), and prostate (11.9%) cancers. Inactivating ATM mutations may |
| | make patients more susceptible to PARP inhibitors. Agents that target ATM and/or |
| | its downstream or upstream effectors are in clinical trials, e.g.: NCT01311713. |
| | In addition, germline mutations in ATM are associated with ataxia-telangiectasia |
| | (also known as Louis-Bar syndrome) and a predisposition to malignancy. |
| BRAF | BRAF encodes a protein belonging to the raf/mil family of serine/threonine protein |
| | kinases. This protein plays a role in regulating the MAP kinase/ERK signaling |
| | pathway initiated by EGFR activation, which affects cell division, differentiation, |
| | and secretion. BRAF somatic mutations have been found in melanoma (43%), |
| | thyroid (39%), biliary tree (14%), colon (12%), and ovarian tumors (12%). Patients |
| | with mutated BRAF genes have a reduced likelihood of response to EGFR targeted |
| | monoclonal antibodies in colorectal cancer. In melanoma, BRAF-mutated patients |
| | are responsive to the BRAF inhibitors, vemurafenib and dabrafenib, and MEK1/2 |
| | inhibitor, trametinib. Various clinical trials (on www.clinicaltrials.gov) |
| | investigating agents which target this gene may be available, which include the |
| | following: NCT01543698, NCT01709292. |
| | BRAF inherited mutations are associated with Noonan/Cardio-Facio-Cutaneous |
| | (CFC) syndrome, syndromes associated with short stature, distinct facial features, |
| | and potential heart/skeletal abnormalities. |
| CDH1 | CDH1 (epithelial cadherin/E-cad) encodes a transmembrane calcium dependent |
| | cell adhesion glycoprotein that plays a major role in epithelial architecture, cell |
| | adhesion and cell invasion. Loss of function of CDH1 contributes to cancer |
| | progression by increasing proliferation, invasion, and/or metastasis. Various |
| | somatic mutations in CDH1 have been identified in diffuse gastric, lobular breast, |
| | endometrial and ovarian carcinomas; the resultant loss of function of E-cad can |
| | contribute to tumor growth and progression. |
| | In addition, germline mutations in CDH1 cause hereditary diffuse gastric cancer |
| | and colorectal cancer; affected women are predisposed to lobular breast cancer |
| | with a risk of about 50%. CDH1 mutation carriers have an estimated cumulative |
| | risk of gastric cancer of 67% for men and 83% for women, by age of 80 years. |
| CDKN2A | CDKN2A or cyclin-dependent kinase inhibitor 2A is a tumor suppressor gene that |
| | encodes two cell cycle regulatory proteins p16INK4A and p14ARF. As upstream |
| | regulators of the retinoblastoma (RB) and p53 signaling pathways, CDKN2A |
| | controls the induction of cell cycle arrest in damaged cells that allows for repair of |
| | DNA. Loss of CDKN2A through whole-gene deletion, point mutation, or promoter |
| | methylation leads to disruption of these regulatory proteins and consequently |
| | dysregulation of growth control. Somatic CDKN2A mutations are documented to |
| | occur in squamous cell lung cancers, head and neck cancer, colorectal cancer, |
| | chronic myelogenous leukemia and malignant pleural mesothelioma. Currently, |
| | there are agents that target downstream of CDKN2A such as CDK4/6 inhibitors |
| | which function by restoring the cell's ability to induce cell cycle arrest. CDK4/6 |
| | inhibitors are in clinical trials for advanced solid tumors, including LEE011 |
| | (NCT01237236) and PD0332991 (NCT01522989, NCT01536743, |
| | NCT01037790). |
| | In addition, germline CDKN2A mutations are associated with melanoma- |
| | pancreatic carcinoma syndrome, which increases the risk for familial malignant |
| | melanoma and pancreatic cancer. |
| c-Kit | c-Kit is a cytokine receptor expressed on the surface of hematopoietic stem cells as |
| | well as other cell types. This receptor binds to stem cell factor (SCF, a cell growth |
| | factor). As c-Kit is a receptor tyrosine kinase, ligand binding causes receptor |
| | dimerization and initiates a phosphorylation cascade resulting in changes in gene |
| | expression. These changes affect proliferation, apoptosis, chemotaxis and |
| | adhesion. C-KIT mutation has been identified in various cancer types including |
| | gastrointestinal stromal tumors (GIST) (up to 85%) and melanoma (7%). c-Kit is |
| | inhibited by multi-targeted agents including imatinib, sunitinib and sorafenib. |
| | Agents which target c-KIT and/or its downstream or upstream effectors are also in |
| | clinical trials for patients carrying c-KIT mutation, e.g.: NCT01028222, |
| | NCT01092728. |
| | In addition, germline mutations in c-KIT have been associated with multiple |
| | gastrointestinal stromal tumors (GIST) and Piebald trait. |
| C-Met | C-Met is a proto-oncogene that encodes the tyrosine kinase receptor of hepatocyte |
| | growth factor (HGF) or scatter factor (SF). c-Met mutation causes aberrant MET |
| | signaling in various cancer types including renal papillary, hepatocellular, head and |
| | neck squamous, gastric carcinomas and non-small cell lung cancer. Activating |
| | point mutations of MET kinase domain can cause cancer of various types, and may |
| | also decrease endocytosis and/or degradation of the receptor, resulting in enhanced |
| | tumor growth and metastasis. Mutations in the juxtamembrane domain (exon 14, |
| | 15) results in the constitutive activation and show enhanced tumorigenicity. c-MET |
| | inhibitors are in clinical trials for patients carrying MET mutations, e.g.: |
| | NCT01121575, NCT00813384. |
| | Germline mutations in c-MET have been associated with hereditary papillary renal |
| | cell carcinoma. |
| CSF1R | CSF1R or colony stimulating factor 1 receptor gene encodes a transmembrane |
| | tyrosine kinase, a member of the CSF1/PDGF receptor family. CSF1R mediates |
| | the cytokine (CSF-1) responsible for macrophage production, differentiation, and |
| | function. Mutations of this gene are associated with hematologic malignancies, as |
| | well as cancers of the liver (21.4%), colon (12.5%), prostate (3.3%), endometrium |
| | (2.4%), and ovary (2.4%). Patients with CSF1R mutations may respond to |
| | imatinib. Agents that target CSF1R and/or its downstream or upstream effectors |
| | are in clinical trials, e.g.: NCT01346358, NCT01440959. |
| | In addition, germline mutations in CSF1R are associated with diffuse |
| | leukoencephalopathy, a rapidly progressive neurodegenerative disorder. |
| CTNNB1 | CTNNB1 or cadherin-associated protein, beta 1, encodes for β-catenin, a central |
| | mediator of the Wnt signaling pathway which regulates cell growth, migration, |
| | differentiation and apoptosis. Mutations in CTNNB1 (often occurring in exon 3) |
| | avert the breakdown of β-catenin, which allows the protein to accumulate resulting |
| | in persistent transactivation of target genes including c-myc and cyclin-D1. |
| | Somatic CTNNB1 mutations account for 1-4% of colorectal cancers, 2-3% of |
| | melanomas, 25-38% of endometrioid ovarian cancers, 84-87% of sporadic desmoid |
| | tumors, as well as the pediatric cancers, hepatoblastoma, medulloblastoma and |
| | Wilms' tumors. Compounds that suppress the Wnt/p-catenin pathway are available |
| | in clinical trials including PRI-724 for advanced solid tumors (NCT01302405) and |
| | LGK974 for melanoma and lobular breast cancer. |
| EGFR | EGFR or epidermal growth factor receptor, is a transmembrane receptor tyrosine |
| | kinase belonging to the ErbB family of receptors. Upon ligand binding, the |
| | activated receptor triggers a series of intracellular pathways (Ras/MAPK, |
| | PI3K/Akt, JAK-STAT) that result in cell proliferation, migration and adhesion. |
| | Dysregulation of EGFR through mutation leads to ligand-independent activation |
| | and constitutive kinase activity, which results in uncontrolled growth and |
| | proliferation of many human cancers. EGFR mutations have been observed in |
| | 20-25% of non-small cell lung cancer (NSCLC), 10% of endometrial and peritoneal |
| | cancers. Somatic gain-of-function EGFR mutations, including in-frame deletions in |
| | exon 19 or point mutations in exon 21, confer sensitivity to first-generation EGFR- |
| | targeted tyrosine kinase inhibitors, whereas the secondary mutation, T790M in |
| | exon 20, confers resistance to tyrosine kinase inhibitors. New agents and |
| | combination therapies that include EGFR TKIs are in clinical trials for primary |
| | treatment of EGFR-mutated patients, including second-generation tyrosine kinase |
| | inhibitors such as icotinib (NCT01665417) for NSCLC or afatinib for advanced |
| | solid tumors (NCT00809133) and lung neoplasms (NCT01466660). In addition, |
| | new therapies and combination therapies are being explored for patients that have |
| | progressed on EGFR-targeted agents including afatinib (NCT01647711) for |
| | NSCLC. |
| | Germline mutations and polymorphisms of EGFR have been associated with |
| | familial lung adeocarcinomas. |
| ERBB2 | ERBB2 (HER2) or v-erb-b2 erythroblastic leukemia viral oncogene homolog 2, |
| | neuro/glioblastoma derived oncogene homolog (avian) encodes a member of the |
| | epidermal growth factor (EGF) receptor family of receptor tyrosine kinases. This |
| | gene binds to other ligand-bound EGF receptor family members to form a |
| | heterodimer and enhances kinase-mediated activation of downstream signaling |
| | pathways, leading to cell proliferation. The most common mechanism for |
| | activation of HER2 is gene amplification, seen in approximately 15% of breast |
| | cancers. Somatic mutations have been found in colon (3.8%), endometrium (3.7%), |
| | prostate (3.0%), ovarian (2.5%), breast (1.7%) gastric (1.9%) cancers and 2-4% of |
| | lung adenocarcinomas. HER2 activated patients may respond to trastuzumab, |
| | afatinib, or lapatinib. Agents that target HER2 are in clinical trials, e.g.: |
| | NCT01306045. |
| ERBB4 | ERBB4 is a member of the Erbb receptor family known to play a pivotal role in |
| | cell-cell signaling and signal transduction regulating cell growth and development. |
| | The most commonly affected signaling pathways are the PI3K-Akt and MAP |
| | kinase pathways. Erbb4 was found to be somatically mutated in 19% of melanomas |
| | and Erbb4 mutations may confer “oncogene addiction” on melanoma cells. Erbb4 |
| | mutations have also been observed in various other cancer types, including, gastric |
| | carcinomas (1.7%), colorectal carcinomas (0.68-2.9%), non-small cell lung cancer |
| | (2.3-4.7%) and breast carcinomas (1.1%), however, their biological impact is not |
| | uniform or consistent across these cancers. Agents that target ERBB4 are in |
| | clinical trials, e.g.: NCT0126408. |
| FBXW7 | FBXW7, or E3 ligase F-box and WD repeat domain containing 7, also known as |
| | Cdc4, encodes three protein isoforms which constitute a component of the |
| | ubiquitin-proteasome complex. Mutation of FBXW7 occurs in hotspots and |
| | disrupts the recognition of and binding with substrates which inhibits the proper |
| | targeting of proteins for degradation (e.g. Cyclin E, c-Myc, SREBP1, c-Jun, Notch- |
| | 1 and mTOR). Mutation frequencies identified in cholangiocarcinomas, T-ALL, |
| | and carcinomas of endometrium, colon and stomach are 35%, 31%, 9%, 9%, and |
| | 6%, respectively. Therapeutic strategies comprise targeting an oncoprotein |
| | downstream of FBXW7, such as mTOR or c-Myc. Tumor cells with mutated |
| | FBXW7 are particularly sensitive to rapamycin treatment, indicating FBXW7 loss |
| | (mutation) can be a predictive biomarker for treatment with inhibitors of the |
| | mTOR pathway. |
| FGFR1 | FGFR1, or fibroblast growth factor receptor 1, encodes for FGFR1 which is |
| | important for cell division, regulation of cell maturation, formation of blood |
| | vessels, wound healing and embryonic development. Somatic activating mutations |
| | have been documented in melanoma, glioblastoma, and lung tumors. Other |
| | aberrations of FGFR1 including protein overexpression and gene amplification are |
| | common in breast cancer, squamous cell lung cancer, colorectal cancer, and, to |
| | some extent in adenocarcinoma of the lung. Recently, it has been shown that |
| | osteosarcoma and advanced solid tumors that exhibit FGFR1 amplification are |
| | sensitive to the pan-FGFR inhibitor, NVP-BGJ398. Other FGFR1-targeted agents |
| | under clinical investigation include dovitinib (NCT01440959). |
| | In addition, germline, gain-of-function mutations in FGFR1 result in |
| | developmental disorders including Kallmann syndrome and Pfeiffer syndrome. |
| FGFR2 | FGFR2 is a receptor for fibroblast growth factor. Activation of FGFR2 through |
| | mutation and amplification has been noted in a number of cancers. Somatic |
| | mutations of the FGFR2 tyrosine kinase have been observed in endometrial |
| | carcinoma, lung squamous cell carcinoma, cervical carcinoma, and melanoma. In |
| | the endometrioid histology of endometrial cancer, the frequency of FGFR2 |
| | mutation is 16% and the mutation is associated with shorter disease free survival in |
| | patients diagnosed with early stage disease. Loss of function FGFR2 mutations |
| | occur in about 8% melanomas and contribute to melanoma pathogenesis. |
| | Functional polymorphisms in the FGFR2 promoter are associated with breast |
| | cancer susceptibility. Agents that target FGFR2 are in clinical trials, e.g.: |
| | NCT01379534. |
| | In addition, germline mutations in FGFR2 are associated with numerous medical |
| | conditions that include congenital craniofacial malformation disorders, Apert |
| | syndrome and the related Pfeiffer and Crouzon syndromes. |
| FGFR3 | FGFR3 or fibroblast growth factor receptor type 3 gene encodes a member of the |
| | FGFR tyrosine kinase family, which include FGFR1, 2, 3, and 4. Dysregulation of |
| | FGFR3 has been implicated in activating the RAS-ERK pathway. FGFR3 has been |
| | found in various malignancies, including bladder cancer and multiple myeloma. |
| | Somatic mutations of this gene have also been found in skin (25.8%), head and |
| | neck (20.0%), and testicular (4.3%) cancers. Studies indicate FGFR3 and PIK3CA |
| | mutations occur together. FGFR3 mutations could serve as a strong prognostic |
| | indicator of a low recurrence rate in bladder cancer. |
| | Agents that target FGFR3 and/or its downstream or upstream effectors are in |
| | clinical trials, e.g.: NCT01004224. |
| | In addition, germline mutations in FGFR3 are associated with achondroplasia, |
| | hypochondroplasia, and Muenke syndrome, disorders involving but not limited to |
| | craniosynostosis and shortened extremities. FGFR3 is also associated with |
| | Crouzon syndrome with acanthosis nigricans. |
| FLT3 | FLT3, or Fms-like tyrosine kinase 3 receptor, is a member of class III receptor |
| | tyrosine kinase family, which includes PDGFRA/B and KIT. Signaling through |
| | FLT3 ligand-receptor complex regulates hematopoiesis, specifically lymphocyte |
| | development. The FLT3 internal tandem duplication (FLT3-ITD) is the most |
| | common genetic lesion in acute myeloid leukemia (AML), occurring in 25% of |
| | cases. FLT3 mutations are as common in solid tumors but have been documented |
| | in breast cancer. Several small molecule multikinase inhibitors targeting the RTK- |
| | III family are in clinical trials, including phase II trials for crenolanib in AML |
| | (NCT01657682), famitinib for nasopharyngeal carcinoma (NCT01462474), |
| | dovitinib for GIST (NCT01440959), and phase I trial for PLX108-01 in solid |
| | tumors (NCT01004861). |
| GNA11 | GNA11 is a proto-oncogene that belongs to the Gq family of the G alpha family of |
| | G protein coupled receptors. Known downstream signaling partners of GNA11 are |
| | phospholipase C beta and RhoA and activation of GNA11 induces MAPK activity. |
| | Over half of uveal melanoma patients lacking a mutation in GNAQ exhibit somatic |
| | mutations in GNA11. Agents that target GNA11 are in clinical trials, e.g.: |
| | NCT01587352, NCT01390818, NCT01143402. |
| GNAQ | GNAQ encodes the Gq alpha subunit of G proteins. G proteins are a family of |
| | heterotrimeric proteins coupling seven-transmembrane domain receptors. |
| | Oncogenic mutations in GNAQ result in a loss of intrinsic GTPase activity, |
| | resulting in a constitutively active Galpha subunit. This results in increased |
| | signaling through the MAPK pathway. Somatic mutations in GNAQ have been |
| | found in 50% of primary uveal melanoma patients and up to 28% of uveal |
| | melanoma metastases. Agents that target GNAQ are in clinical trials, e.g.: |
| | NCT01587352, NCT01390818, NCT01143402. |
| GNAS | GNAS (or GNAS complex locus) encodes a stimulatory G protein alpha-subunit. |
| | These guanine nucleotide binding proteins (G proteins) are a family of |
| | heterotrimeric proteins which couple seven-transmembrane domain receptors to |
| | intracellular cascades. Stimulatory G-protein alpha-subunit transmits hormonal and |
| | growth factor signals to effector proteins and is involved in the activation of |
| | adenylate cyclases. Mutations of GNAS gene at codons 201 or 227 lead to |
| | constitutive cAMP signaling. GNAS somatic mutations have been found in |
| | pituitary (27.9%), pancreatic (19.2%), ovarian (11.4%), adrenal gland (6.2%), and |
| | colon (6.0%) cancers. SNPs in GNAS1 are a predictive marker for tumor response |
| | in cisplatin/fluorouracil-based radiochemotherapy in esophageal cancer. |
| | In addition, germline mutations of GNAS have been shown to be the cause of |
| | McCune-Albright syndrome (MAS), a disorder marked by endocrine, |
| | dermatologic, and bone abnormalities. GNAS is usually found as a mosaic |
| | mutation in patients. Loss of function mutations are associated with |
| | pseudohypoparathyroidism and pseudopseudohypoparathyroidism. |
| HNF1A | HNF1A, or hepatocyte nuclear factor 1 homeobox A, encodes a transcription factor |
| | that is highly expressed in the liver, found on chromosome 12. It regulates a large |
| | number of genes, including those for albumin, alpha1 -antitrypsin, and fibrinogen. |
| | HNF1A has been associated with an increased risk of pancreatic cancer. HNF1A |
| | somatic mutations are found in liver (30.1%), colon (14.5%), endometrium |
| | (11.1%), and ovarian (2.5%) cancers. |
| | In addition, germline mutations of HNF1A are associated with maturity-onset |
| | diabetes of the young type 3. |
| HRAS | HRAS (homologous to the oncogene of the Harvey rat sarcoma virus), together |
| | with KRAS and NRAS, belong to the superfamily of RAS GTPase. RAS protein |
| | activates RAS-MEK-ERK/MAPK kinase cascade and controls intracellular |
| | signaling pathways involved in fundamental cellular processes such as |
| | proliferation, differentiation, and apoptosis. Mutant Ras proteins are persistently |
| | GTP-bound and active, causing severe dysregulation of the effector signaling. |
| | HRAS mutations have been identified in cancers from the urinary tract (10%-40%), |
| | skin (6%) and thyroid (4%) and they account for 3% of all RAS mutations |
| | identified in cancer. RAS mutations (especially HRAS mutations) occur (5%) in |
| | cutaneous squamous cell carcinomas and keratoacanthomas that develop in patients |
| | treated with BRAF inhibitor vemurafenib, likely due to the paradoxical activation |
| | of the MAPK pathway. Agents that target HRAS and/or its downstream or |
| | upstream effectors are in clinical trials, e.g.: NCT01306045. |
| | In addition, germline mutation in HRAS has been associated with Costello |
| | syndrome, a genetic disorder that is characterized by delayed development and |
| | mental retardation and distinctive facial features and heart abnormalities. |
| IDH1 | IDH1 encodes for isocitrate dehydrogenase in cytoplasm and is found to be |
| | mutated in ~5% of primary gliomas and 60-90% of secondary gliomas, as well as |
| | in 12-18% of patients with acute myeloid leukemia. Mutated IDH1 results in |
| | impaired catalytic function of the enzyme, thus altering normal physiology of |
| | cellular respiration and metabolism. Furthermore, this mutation results in |
| | tumorigenesis. In gliomas, IDH1 mutations are associated with lower-grade |
| | astrocytomas and oligodendrogliomas (grade II/III). IDH gene mutations are |
| | associated with markedly better survival in patients diagnosed with malignant |
| | astrocytoma; and clinical data support a more aggressive surgery for IDH1 mutated |
| | patients because these individuals may be able to achieve long-term survival. In |
| | contrast, IDH1 mutation is associated with a worse prognosis in AML. In low- |
| | grade glioma patients receiving temozolomide before anaplastic transformation, |
| | IDH mutations (IDH1 and IDH2) have been shown to predict response to |
| | temozolomide. Agents that target IDH and/or its downstream or upstream effectors |
| | are in clinical trials, e.g.: NCT01534845. |
| JAK2 | JAK2 or Janus kinase 2 is a part of the JAK/STAT pathway which mediates |
| | multiple cellular responses to cytokines and growth factors including proliferation |
| | and cell survival. It is also essential for numerous developmental and homeostatic |
| | processes, including hematopoiesis and immune cell development. Mutations in the |
| | JAK2 kinase domain result in constitutive activation of the kinase and the |
| | development of chronic myeloproliferative neoplasms such as polycythemia vera |
| | (95%), essential thrombocythemia (50%) and myelofibrosis (50%). JAK2 |
| | mutations were also found in BCR-ABL1-negative acute lymphoblastic leukemia |
| | patients and the mutated patients show a poor outcome. Agents that target JAK2 |
| | and/or its downstream or upstream effectors are in clinical trials for patients |
| | carrying JAK2 mutations, e.g.: NCT00668421, NCT01038856. |
| | In addition, germline mutations in JAK2 have been associated with |
| | myeloproliferative neoplasms and thrombocythemia. |
| JAK3 | JAK3 or Janus activated kinase 3 is an intracellular tyrosine kinase involved in |
| | cytokine signaling, while interacting with members of the STAT family. Like |
| | JAK1, JAK2, and TYK2, JAK3 is a member of the JAK family of kinases. When |
| | activated, kinase enzymes phosphorylate one or more signal transducer and |
| | activator of transcription (STAT) factors, which translocate to the cell nucleus and |
| | regulate the expression of genes associated with survival and proliferation. JAK3 |
| | signaling is related to T cell development and proliferation. This biomarker is |
| | found in malignancies like head and neck (20.8%) colon (7.2%), prostate (4.8%), |
| | ovary (3.5%), breast (1.7%), lung (1.2%), and stomach (0.6%) cancer. |
| | In addition, germline mutations of JAK3 are associated with severe, combined |
| | immunodeficiency disease (SCID). |
| KDR | KDR (VEGFR2) or Kinase insert domain receptor gene, also known as vascular |
| | endothelial growth factor receptor-2 (VEGFR2), is involved with angiogenesis and |
| | is expressed on almost all endothelial cells. VEGF ligands bind to KDR, which |
| | leads to receptor dimerization and signal transduction. Somatic mutations in KDR |
| | have been observed in angiosarcoma (10.0%), and colon (12.7%), skin (12.7%), |
| | gastric (5.3%), lung (3.2%), renal (2.3%), and ovarian (1.9%) cancers. VEGFR |
| | antagonists that are FDA-approved or in clinical trials include bevacizumab, |
| | regorafenib, pazopanib, and vandetanib. Additional agents that target KDR and/or |
| | its downstream or upstream effectors are in clinical trials, e.g.: NCT01068587. |
| KRAS | KRAS, or V-Ki-ras2 Kirsten rat sarcoma viral oncogene homolog, encodes a |
| | signaling intermediate involved in many signaling cascades including the EGFR |
| | pathway. KRAS somatic mutations have been found in pancreatic (57.4%), colon |
| | (34.9%), lung (16.0%), biliary tract (28.2%), and endometrial (14.6%) cancers. |
| | Mutations at activating hotspots are associated with resistance to EGFR tyrosine |
| | kinase inhibitors (e.g., erlotinib, gefitinib) and monoclonal antibodies (e.g., |
| | cetuximab, panitumumab). Agents that target KRAS are in clinical trials, e.g.: |
| | NCT01248247, NCT01229150. |
| | In addition, germline mutations of KRAS (VI41, T58I, and D153V amino acid |
| | substitutions) are associated with Noonan syndrome. |
| MLH1 | MLH1 or mutL homolog 1, colon cancer, nonpolyposis type 2 (E. coli) gene |
| | encodes a mismatch repair (MMR) protein which repairs DNA mismatches that |
| | occur during replication. Although the frequency is higher in colon cancer (10.4%), |
| | MLH1 somatic mutations have been found in esophageal (6.4%), ovarian (5.4%), |
| | urinary tract (5.3%), pancreatic (5.2%), and prostate (4.7%) cancers. Germline |
| | mutations of MLH1 are associated with Lynch syndrome, also known as hereditary |
| | non-polyposis colorectal cancer (HNPCC). Patients with Lynch syndrome are at |
| | increased risk for various malignancies, including intestinal, gynecologic, and |
| | upper urinary tract cancers and in its variant, Muir-Torre syndrome, with sebaceous |
| | tumors. |
| MPL | MPL or myeloproliferative leukemia gene encodes the thrombopoietin receptor, |
| | which is the main humoral regulator of thrombopoiesis in humans. MPL mutations |
| | cause constitutive activation of JAK-STAT signaling and have been detected in |
| | 5-7% of patients with primary myelofibrosis (PMF) and 1% of those with essential |
| | thrombocythemia (ET). |
| | In addition, germline mutations in MPL (S505N) have been associated with |
| | familial thrombocythemia. |
| NOTCH1 | NOTCH1, or notch homolog 1, translocation-associated, encodes a member of the |
| | Notch signaling network, an evolutionary conserved pathway that regulates |
| | developmental processes by regulating interactions between physically adjacent |
| | cells. Notch signaling modulates interplay between tumor cells, stromal matrix, |
| | endothelial cells and immune cells, and mutations in NOTCH1 play a central role |
| | in disruption of microenvironmental communication, potentially leading to cancer |
| | progression. Due to the dual, bi-directional signaling of NOTCH1, activating |
| | mutations have been found in ALL and CLL, however loss of function mutations |
| | in NOTCH1 are prevalent in 11-15% of HNSCC. NOTCH1 mutations have also |
| | been found in 2% of glioblastomas, ~1% of ovarian cancers, 10% lung |
| | adenocarcinomas, 8% of squamous cell lung cancers and 5% of breast cancers. |
| | Notch pathway-directed therapy approaches differ depending on whether the tumor |
| | harbors gain or loss of function mutations, thus are classified as Notch pathway |
| | inhibitors or activators, respectively. Notch pathway modulators are being |
| | investigated in clinical trials, including MK0752 for advanced solid tumors |
| | (NCT01295632) and panobinostat (LBH589) for various refractory hematologic |
| | malignancies and many types of solid tumors including thyroid cancer |
| | (NCT01013597) and melanoma (NCT01065467). |
| NPM1 | NPM1, or nucleophosmin, is a nucleolar phosphoprotein belonging to a family of |
| | nuclear chaperones with proliferative and growth-suppressive roles. In several |
| | hematological malignancies, the NPM locus is lost or translocated, leading to |
| | expression of oncogenic proteins. NPM1 is mutated in one-third of patients with |
| | adult AML and leads to aberrant localization in the cytoplasm leading to activation |
| | of downstream pathways including JAK/STAT, RAS/ERK, and PI3K, leading to |
| | cell proliferation, survival and cytoskeletal rearrangements. In addition, the most |
| | common translocation in anaplastic large cell lymphoma (ALCL) is the NPM-ALK |
| | translocation which leads to expression of an oncogenic fusion protein with |
| | constitutive kinase activity. AML cells with mutant NPM are more sensitive to |
| | some chemotherapeutic agents including daunorubicin and camptothecin. ALK- |
| | targeted therapies such as crizotinib are under clinical investigation for ALK-NPM |
| | positive ALCL (NCT00939770). |
| NRAS | NRAS is an oncogene and a member of the (GTPase) ras family, which includes |
| | KRAS and HRAS. This biomarker has been detected in multiple cancers including |
| | melanoma (15%), colorectal cancer (4%), AML (10%) and bladder cancer (2%). |
| | Acquired mutations in NRAS may be associated with resistance to vemurafenib in |
| | melanoma patients. In colorectal cancer patients NRAS mutation is associated with |
| | resistance to EGFR-targeted monoclonal antibodies. Agents which target this gene |
| | and/or its downstream or upstream effectors are in clinical trials, e.g.: |
| | NCT01306045, NCT01320085 |
| | In addition, germline mutations in NRAS have been associated with Noonan |
| | syndrome, autoimmune lymphoproliferative syndrome and juvenile |
| | myelomonocytic leukemia. |
| PDGFRA | PDGFRA is the alpha subunit of platelet-derived growth factor receptor, a surface |
| | tyrosine kinase receptor, which can activate multiple signaling pathways including |
| | PIK3CA/AKT, RAS/MAPK and JAK/STAT. PDGFRA mutations are found in |
| | 5-8% of gastrointestinal stromal tumor cases, and in 40-50% of KIT wild type |
| | GISTs. Gain of function PDGFRA mutations confer imatinib sensitivity, while |
| | substitution mutation in exon 18 (D842V) shows resistance to the drug. A |
| | PDGFRA mutation in the extracellular domain was shown to identify a subgroup |
| | of DIPG (diffuse intrinsic pontine glioma) patients with significantly worse |
| | outcome PDGFRA inhibitors (e.g., crenolanib, pazopanib) are in clinical trials for |
| | patients carrying PDGFRA mutations, e.g.: NCT01243346, NCT01524848, |
| | NCT01478373. |
| | In addition, germline mutations in PDGFRA have been associated with Familial |
| | gastrointestinal stromal tumors and Hypereosinophillic Syndrome (HES). |
| PIK3CA | PIK3CA or phosphoinositide-3-kinase catalytic alpha polypeptide encodes a |
| | protein in the PI3 kinase pathway. This pathway is an active target for drug |
| | development. PIK3CA somatic mutations have been found in breast (26.1%), |
| | endometrial (23.3%), urinary tract (19.3%), colon (13.0%), and ovarian (10.8%) |
| | cancers. Somatic mosaic activating mutations in PIK3CA may cause CLOVES |
| | syndrome. PIK3CA mutations have been associated with benefit from mTOR |
| | inhibitors (e.g., everolimus, temsirolimus). Breast cancer patients with activation of |
| | the PI3K pathway due to PTEN loss or PIK3CA mutation/amplification may have |
| | a shorter survival following trastuzumab treatment. PIK3CA mutated (exon 20) |
| | colorectal cancer patients are less likely to respond to EGFR targeted monoclonal |
| | antibody therapy. Agents that target PIK3CA are in clinical trials, e.g.: |
| | NCT00877773, NCT01277757, NCT01219699, NCT01501604. |
| PTEN | PTEN, or phosphatase and tensin homolog, is a tumor suppressor gene that |
| | prevents cells from proliferating. PTEN is an important mediator in signaling |
| | downstream of EGFR, and loss of PTEN gene function/expression due to gene |
| | mutations or allele loss is associated with reduced benefit to EGFR-targeted |
| | monoclonal antibodies. Mutation in PTEN is found in 5-14% of colorectal cancer |
| | and 7% of breast cancer. PTEN mutation is generally related to loss of function of |
| | the encoded phosphatase, and an upregulation of the PIK3CA/AKT pathway. The |
| | role of PTEN loss in response to PIK3CA and mTOR inhibitors has been evaluated |
| | in some clinical studies. Agents that target PTEN and/or its downstream or |
| | upstream effectors are in clinical trials, including the following: NCT01430572, |
| | NCT01306045. |
| | In addition, germline PTEN mutations associate with Cowden disease and |
| | Bannayan-Riley-Ruvalcaba syndrome. These dominantly inherited disorders |
| | belong to a family of hamartomatous polyposis syndromes which feature multiple |
| | tumor-like growths (hamartomas) accompanied by an increased risk of breast |
| | carcinoma, follicular carcinoma of the thyroid, glioma, prostate and endometrial |
| | cancer. Trichilemmoma, a benign, multifocal neoplasm of the skin is also |
| | associated with PTEN germline mutations. |
| PTPN11 | PTPN11, or tyrosine-protein phosphatase non-receptor type 11, is a proto- |
| | oncogene that encodes a signaling molecule, Shp-2, which regulates various cell |
| | functions like mitogenic activation and transcription regulation. PTPN11 gain-of- |
| | function somatic mutations have been found to induce hyperactivation of the Akt |
| | and MAPK networks. Because of this hyperactivation, Ras effectors such as Mek |
| | and PI3K are targets for candidate therapies in those with PTPN11 gain-of-function |
| | mutations. PTPN11 somatic mutations are found in hematologic and lymphoid |
| | malignancies (8%), gastric (2.4%), colon (2%), ovarian (1.7%), and soft tissue |
| | (1.6%) cancers. |
| | In addition, germline mutations of PTPN11 are associated with Noonan syndrome, |
| | which itself is associated with juvenile myelomonocytic leukemia (JMML). |
| | PTPN11 is also associated with LEOPARD syndrome, which is associated with |
| | neuroblastoma and myeloid leukemia. |
| RB1 | RB1, or retinoblastoma-1, is a tumor suppressor gene whose protein regulates the |
| | cell cycle by interacting with various transcription factors, including the E2F |
| | family (which controls the expression of genes involved in the transition of cell |
| | cycle checkpoints). RB1 mutations have also been detected in ocular and other |
| | malignancies, such as ovarian (10.4%), bladder (41.3%), prostate (8.2%), breast |
| | (6.1%), brain (5.6%), colon (5.3%), and renal (1.5%) cancers. RB1 status, along |
| | with other mitotic checkpoints, has been associated with the prognosis of GIST |
| | patients. |
| | In addition, germline mutations of RB1 are associated with the pediatric tumor, |
| | retinoblastoma. Inherited retinoblastoma is usually bilateral. Patients with a history |
| | of retinoblastoma are at increased risk for secondary malignancies. |
| RET | RET or rearranged during transfection gene, located on chromosome 10, activates |
| | cell signaling pathways involved in proliferation and cell survival. RET mutations |
| | are mostly found in papillary thyroid cancers and medullary thyroid cancers |
| | (MTC), but RET fusions have also been found in 1% of lung adenocarcinomas. A |
| | 10-year study notes that medullary thyroid cancer patients with somatic mutations |
| | of RET correlate with a poor prognosis. Approximately 50% of patients with |
| | sporadic MTC have somatic RET mutations; 85% of these involve the M918T |
| | mutation, which is associated with a higher response rate to vandetanib in |
| | comparison to M918T negative patients. Agents that target RET are in clinical |
| | trials, e.g.: NCT00514046, NCT01582191. |
| | Germline activating mutations of RET are associated with multiple endocrine |
| | neoplasia type 2 (MEN2), which is characterized by the presence of medullary |
| | thyroid carcinoma, bilateral pheochromocytoma, and primary hyperparathyroidism. |
| | Germline inactivating mutations of RET are associated with Hirschsprung's |
| | disease. |
| SMAD4 | SMAD4, or mothers against decapentaplegic homolog 4, is one of eight proteins in |
| | the SMAD family, whose members are involved in multiple signaling pathways |
| | and are key modulators of the transcriptional responses to the transforming growth |
| | factor-β (TGFβ) receptor kinase complex. SMAD4 resides on chromosome 18q21, |
| | one of the most frequently deleted chromosomal regions in colorectal cancer. |
| | Smad4 stabilizes Smad DNA-binding complexes and also recruits transcriptional |
| | coactivators such as histone acetyltransferases to regulatory elements. |
| | Dysregulation of SMAD4 may occur late in tumor development, and can occur |
| | through mutations of the MH1 domain which inhibits the DNA-binding function, |
| | thus dysregulating TGFβR signaling. Mutated (inactivated) SMAD4 is found in |
| | 50% of pancreatic cancers and 10-35% of colorectal cancers. Studies have shown |
| | that preservation of SMAD4 through retention of the 18q21 region, leads to clinical |
| | benefit from 5-fluorouracil-based therapy. In addition, various clinical trials |
| | investigating agents which target the TGFβR signaling axis are available including |
| | PF-03446962 for advanced solid tumors including NCT00557856. |
| | In addition, germline mutations in SMAD4 are associated with juvenile polyposis |
| | (JP) and combined syndrome of JP and hereditary hemorrhagic teleangiectasia (JP- |
| | HHT). |
| SMARCB1 | SMARCB1 also known as SWI/SNF related, matrix associated, actin dependent |
| | regulator of chromatin, subfamily b, member 1, is a tumor suppressor gene |
| | implicated in cell growth and development. Loss of expression of SMARCB1 has |
| | been observed in tumors including epithelioid sarcoma, renal medullary carcinoma, |
| | undifferentiated pediatric sarcomas, and a subset of hepatoblastomas. |
| | In addition, germline mutation in SMARCB1 causes about 20% of all rhabdoid |
| | tumors which makes it important for clinicians to facilitate genetic testing and refer |
| | families for genetic counseling. Germline SMARCB1 mutations have also been |
| | identified as the pathogenic cause of a subset of schwannomas and meningiomas. |
| SMO | SMO (smoothened) is a G protein-coupled receptor which plays an important role |
| | in the Hedgehog signaling pathway. It is a key regulator of cell growth and |
| | differentiation during development, and is important in epithelial and mesenchymal |
| | interaction in many tissues during embryogenesis. Dysregulation of the Hedgehog |
| | pathway is found in cancers including basal cell carcinomas (12%) and |
| | medulloblastoma (1%). A gain-of-function mutation in SMO results in constitutive |
| | activation of hedgehog pathway signaling, contributing to the genesis of basal cell |
| | carcinoma. SMO mutations have been associated with the resistance to SMO |
| | antagonist GDC-0449 in medulloblastoma patients. SMO mutation may also |
| | contribute to resistance to SMO antagonist LDE225 in BCC. SMO antagonists are |
| | in clinical trials, e.g.: NCT01529450. |
| SRC | SRC, or c-Src is a non-receptor tyrosine kinase, plays a critical role in cellular |
| | growth, proliferation, adhesion and angiogenesis. Normally maintained in a |
| | repressed state by intramolecular interactions involving the SH2 and SH3 domains, |
| | Src mutation prevents these restrictive intramolecular interactions, conferring a |
| | constitutively active state. Mutations are found in 12% of colon cancers (especially |
| | those metastatic to the liver) and 1-2% of endometrial cancers. Agents that target |
| | SRC are in clinical trials, e.g.: dasatinib for treatment of GIST (NCT01643278), |
| | endometrial cancer (NCT01440998), and other solid tumors (NCT01445509); |
| | saracatinib (AZD0530) for breast (NCT01216176) and pancreatic (NCT00735917) |
| | cancers; and bosutinib (SKI-606) for glioblastoma (NCT01331291). |
| STK11 | STK11, also known as LKB1, is a serine/threonine kinase. It is thought to be a tumor suppressor |
| | gene which acts by interacting with p53 and CDC42. It modulates the activity of AMP-activated |
| | protein kinase, causes inhibition of mTOR, regulates cell polarity, inhibits the cell cycle, and |
| | activates p53. Somatic mutations in STK11are associated with a history of smoking and KRAS |
| | mutation in NSCLC patients. The frequency of STK11 mutation in lung adenocarcinomas |
| | ranges from 7%-30%. STK11 loss may play a role in development of metastatic disease in lung |
| | cancer patients. Mutations of this gene also drive progression of HPV-induced dysplasia to |
| | invasive, cervical cancer and hence STK11 status may be exploited clinically to predict the |
| | likelihood of disease recurrence. Agents that target STK11are in clinical trials, e.g.: |
| | NCT01578551. |
| | In addition, germline mutations in STK11 are associated with Peutz-Jeghers |
| | syndrome which is characterized by early onset hamartomatous gastro-intestinal |
| | polyps and increased risk of breast, colon, gastric and ovarian cancer. |
| TP53 | TP53, or p53, plays a central role in modulating response to cellular stress through |
| | transcriptional regulation of genes involved in cell-cycle arrest, DNA repair, |
| | apoptosis, and senescence. Inactivation of the p53 pathway is essential for the |
| | formation of the majority of human tumors. Mutation in p53 (TP53) remains one of |
| | the most commonly described genetic events in human neoplasia, estimated to |
| | occur in 30-50% of all cancers with the highest mutation rates occurring in head |
| | and neck squamous cell carcinoma and colorectal cancer. Generally, presence of a |
| | disruptive p53 mutation is associated with a poor prognosis in all types of cancers, |
| | and diminished sensitivity to radiation and chemotherapy. Agents are in clinical |
| | trials which target p53's downstream or upstream effectors. Utility may depend on |
| | the p53 status. For p53 mutated patients, Chk1 inhibitors in advanced cancer |
| | (NCT01115790) and Wee1 inhibitors in refractory ovarian cancer (NCT01164995) |
| | are being investigated. For p53 wildtype patients with sarcoma, mdm2 inhibitors |
| | (NCT01605526) are being investigated. |
| | In addition, germline p53 mutations are associated with the Li-Fraumeni syndrome |
| | (LFS) which may lead to early-onset of several forms of cancer currently known to |
| | occur in the syndrome, including sarcomas of the bone and soft tissues, carcinomas |
| | of the breast and adrenal cortex (hereditary adrenocortical carcinoma), brain |
| | tumors and acute leukemias. |
| VHL | VHL or von Hippel-Lindau gene encodes for tumor suppressor protein pVHL, |
| | which polyubiquitylates hypoxia-inducible factor in an oxygen dependent manner. |
| | Absence of pVHL causes stabilization of HIF and expression of its target genes, |
| | many of which are important in regulating angiogenesis, cell growth and cell |
| | survival. VHL somatic mutation has been seen in 20-70% of patients with sporadic |
| | clear cell renal cell carcinoma (ccRCC) and the mutation may imply a poor |
| | prognosis, adverse pathological features, and increased tumor grade or lymph-node |
| | involvement. Renal cell cancer patients with a ‘loss of function’ mutation in VHL |
| | show a higher response rate to therapy (bevacizumab or sorafenib) than is seen in |
| | patients with wild type VHL. Agents which target VHL and/or its downstream or |
| | upstream effectors are in clinical trials, e.g.: NCT01538238. |
| | In addition, germline mutations in VHL cause von Hippel-Lindau syndrome, |
| | associated with clear-cell renal-cell carcinomas, central nervous system |
| | hemangioblastomas, pheochromocytomas and pancreatic tumors. |

In an aspect, the invention provides a molecular profile for a cancer which comprises mutational analysis of a panel of genes, e.g., at least 2, 3, 4, 5, 6, 7, 8, 9, 10, 12, 15, 20, 25, 30, 35, 40, 45 or at least 50 genes. As described herein, the molecular profile can be used to identify a candidate agent that is likely to benefit the cancer patient. The molecular profile can also be used to identify a candidate agent that is not likely to benefit the cancer patient. Further as described, a report can be generated that describes results of the molecular profile. The report may include a summary of the mutational analysis for the genes assessed. The report may also provide a linkage of the mutational analysis with the predicted efficacy of various treatments based on the mutational analysis. Such rules for mutation—drug association are provided herein, e.g., in Table 25 or any of Tables 7-24. The report may also comprise one or more clinical trials associated with one or more identified mutation in the patient. Mutational analysis can also be used to detect mutations of genes that are known to affect a prognosis or provide other characterization of a cancer.

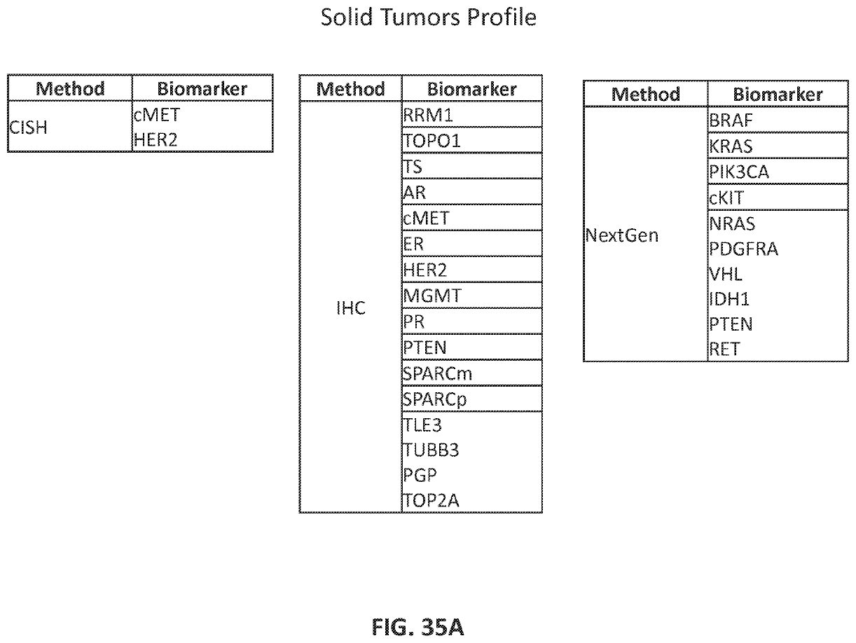

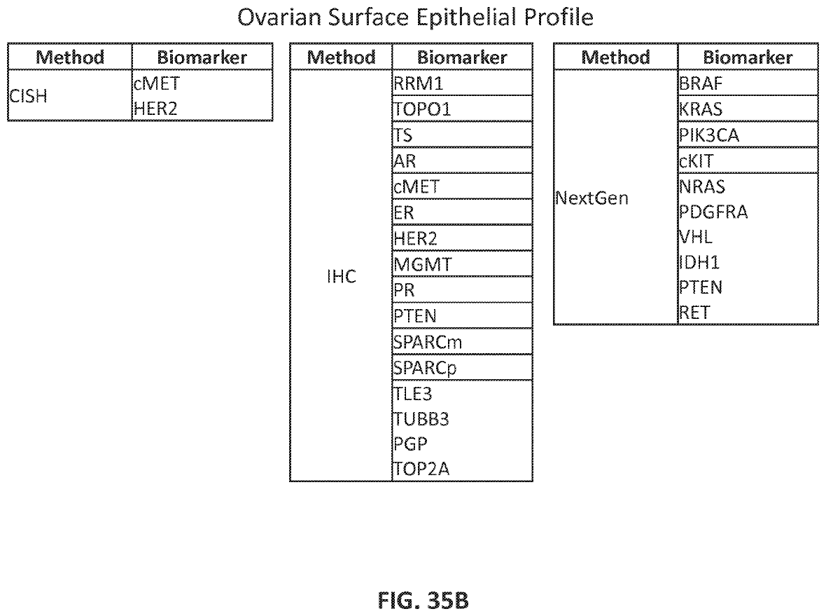

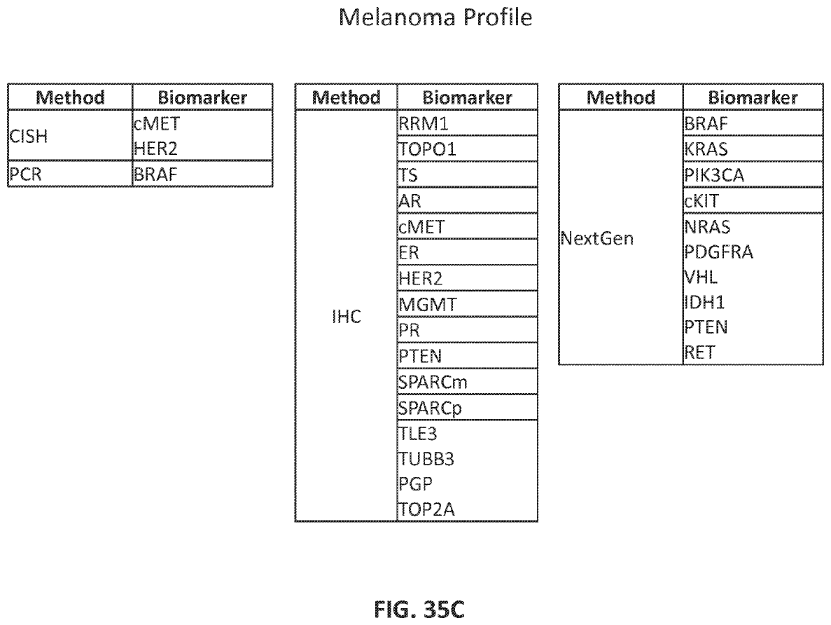

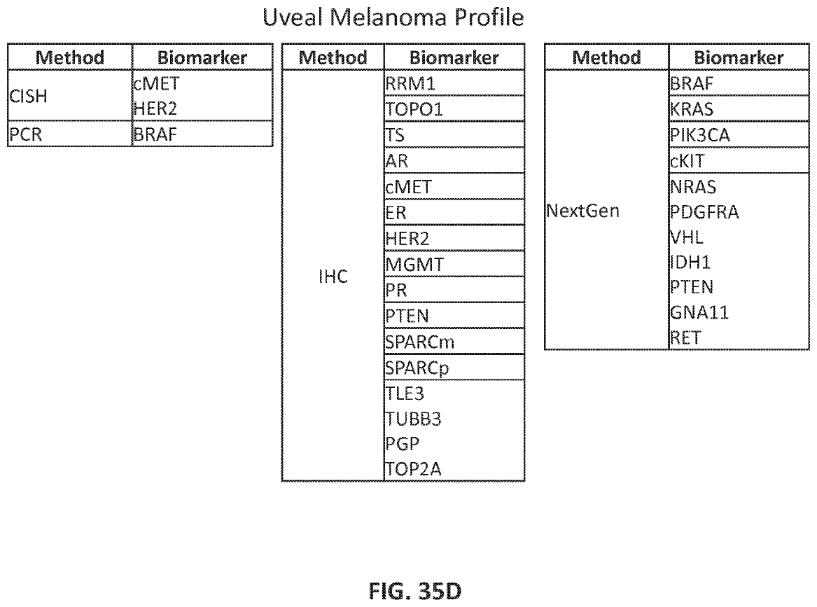

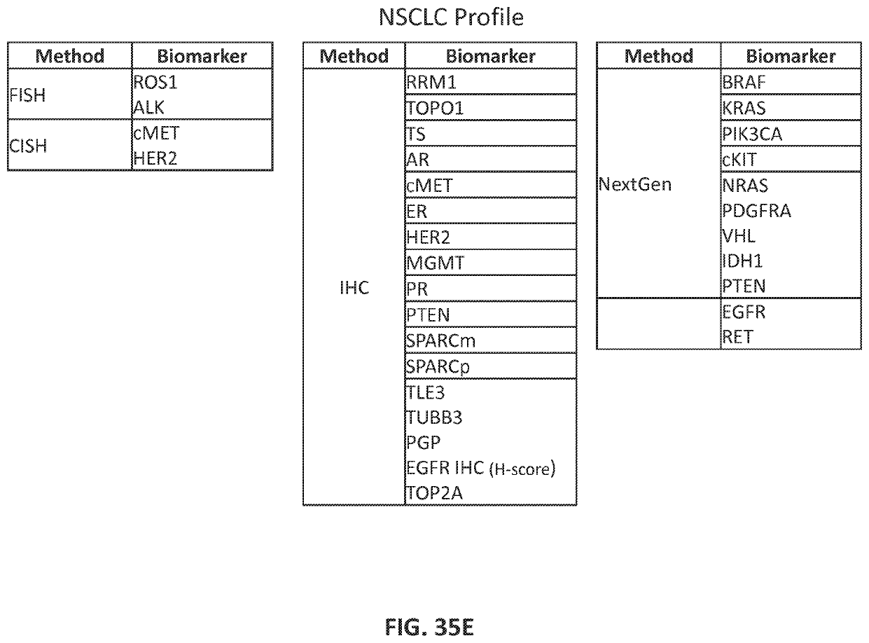

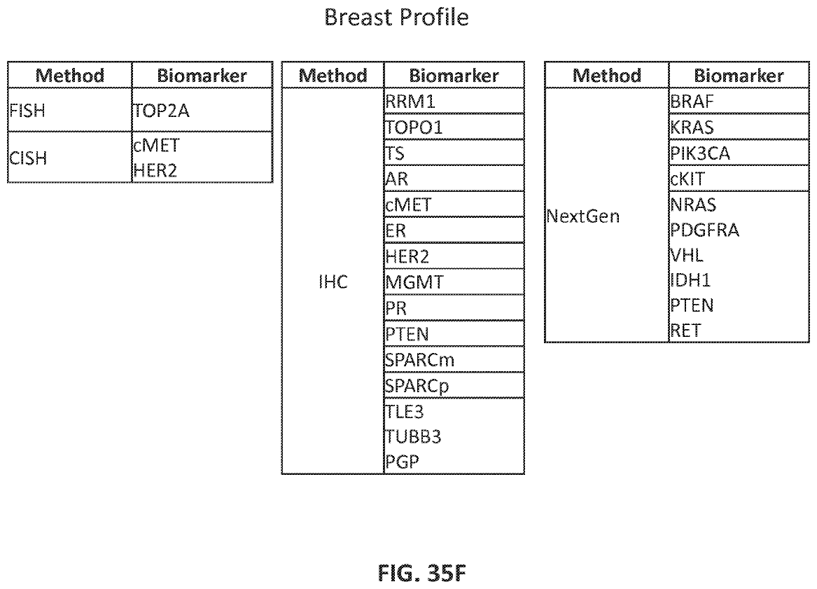

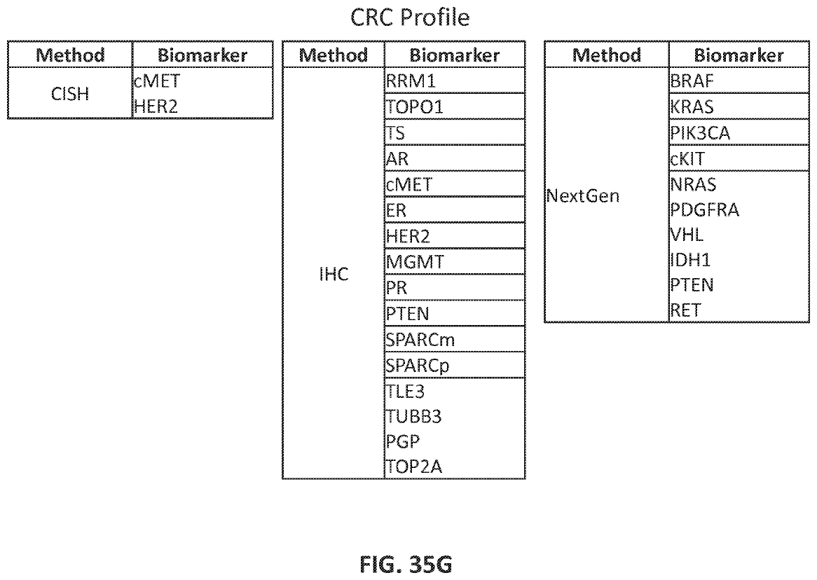

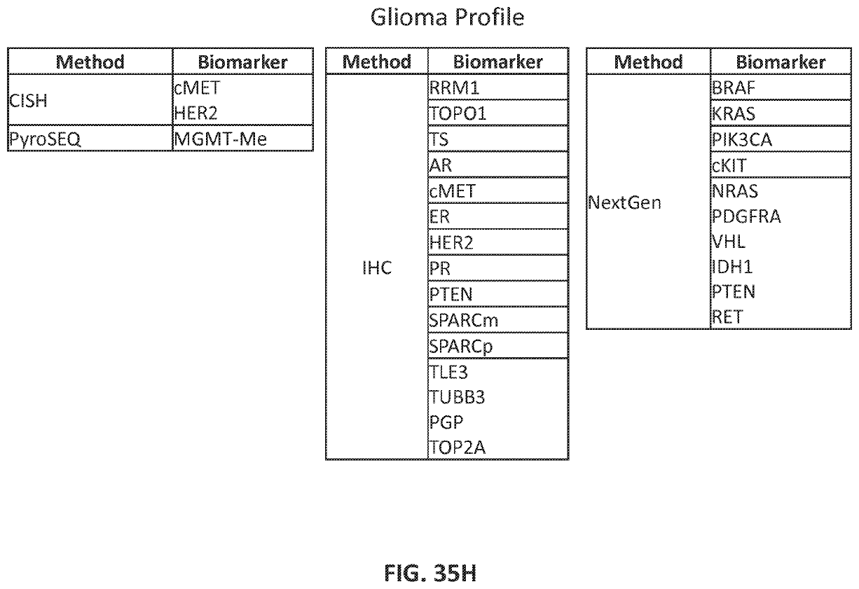

The molecular profile may comprise mutational analysis of one or more gene in Table 25. For example, the molecular profile may include the mutational analysis of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or at least 50 genes in Table 25. The molecular profile may include the mutational analysis of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 or ABL1, AKT1, ALK, APC, ATM, BRAF, CDH1, CDKN2A, c-Kit, C-Met, CSF1R, CTNNB1, EGFR, ERBB2, ERBB4, FBXW7, FGFR1, FGFR2, FGFR3, FLT3, GNA11, GNAQ, GNAS, HNF1A, HRAS, IDH1, JAK2, JAK3, KDR, KRAS, MLH1, MPL, NOTCH1, NPM1, NRAS, PDGFRA, PIK3CA, PTEN, PTPN11, RB1, RET, SMAD4, SMARCB1, SMO, SRC, STK11, TP53, VHL. In an embodiment, the molecular profile comprises mutational analysis of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 of ABL1, AKT1, ALK, APC, ATM, BRAF, CDH1, CSF1R, CTNNB1, EGFR, ERBB2 (HER2), ERBB4, FBXW7, FGFR1, FGFR2, FLT3, GNA11, GNAQ, GNAS, HNF1A, HRAS, IDH1, JAK2, JAK3, KDR (VEGFR2), KIT, KRAS, MET, MLH1, MPL, NOTCH1, NPM1, NRAS, PDGFRA, PIK3CA, PTEN, PTPN11, RB1, RET, SMAD4, SMARCB1, SMO, STK11, TP53, VHL. For example, the molecular profile may comprise mutational analysis of ABL1, AKT1, ALK, APC, ATM, BRAF, CDH1, CSF1R, CTNNB1, EGFR, ERBB2 (HER2), ERBB4, FBXW7, FGFR1, FGFR2, FLT3, GNA11, GNAS, HNF1A, HRAS, IDH1, JAK2, JAK3, KDR (VEGFR2), KIT, KRAS, MET, MLH1, MPL, NOTCH1, NPM1, NRAS, PDGFRA, PIK3CA, PTEN, PTPN11, RB1, RET, SMAD4, SMARCB1, SMO, STK11, TP53, and VHL. In an embodiment, the mutational analysis molecular profile is performed in concert with another molecular profile provided herein. For example, the analysis of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 of ABL1, AKT1, ALK, APC, ATM, BRAF, CDH1, CSF1R, CTNNB1, EGFR, ERBB2 (HER2), ERBB4, FBXW7, FGFR1, FGFR2, FLT3, GNA11, GNAS, HNF1A, HRAS, IDH1, JAK2, JAK3, KDR (VEGFR2), KIT, KRAS, MET, MLH1, MPL, NOTCH1, NPM1, NRAS, PDGFRA, PIK3CA, PTEN, PTPN11, RB1, RET, SMAD4, SMARCB1, SMO, STK11, TP53 and VHL can be reported together with the molecular profiling described in any of FIGS. 33A-Q, FIGS. 35A-I and/or Tables 7-25. In an embodiment, the mutational analysis of ABL1, AKT1, ALK, APC, ATM, BRAF, CDH1, CSF1R, CTNNB1, EGFR, ERBB2 (HER2), ERBB4, FBXW7, FGFR1, FGFR2, FLT3, GNA11, GNAS, HNF1A, HRAS, IDH1, JAK2, JAK3, KDR (VEGFR2), KIT, KRAS, MET, MLH1, MPL, NOTCH1, NPM1, NRAS, PDGFRA, PIK3CA, PTEN, PTPN11, RB1, RET, SMAD4, SMARCB1, SMO, STK11, TP53 and VHL genes is reported together with the molecular profiling described in any of FIGS. 33A-Q, FIGS. 35A-I and/or Tables 7-25.

In an embodiment, the molecular profile comprises mutational analysis of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33 or 34 of ABL1, AKT1, ALK, APC, ATM, BRAF, cKIT, cMET, CSF1R, CTNNB1, EGFR, ERBB2, FGFR1, FGFR2, FLT3, GNA11, GNAQ, GNAS, HRAS, IDH1, JAK2, KDR (VEGFR2), KRAS, MLH1, MPL, NOTCH1, NRAS, PDGFRA, PIK3CA, PTEN, RET, SMO, TP53, VHL. For example, ABL1, AKT1, ALK, APC, ATM, BRAF, cKIT, cMET, CSF1R, CTNNB1, EGFR, ERBB2, FGFR1, FGFR2, FLT3, GNA11, GNAQ, GNAS, HRAS, IDH1, JAK2, KDR (VEGFR2), KRAS, MLH1, MPL, NOTCH1, NRAS, PDGFRA, PIK3CA, PTEN, RET, SMO, TP53, VHL may be assessed. As desired, additional biomarkers may be assessed for mutational analysis including at least 1, 2, 3, 4, 5, 6, 7, 8, 9, 10 or 11 of CDH1, ERBB4, FBXW7, HNF1A, JAK3, NPM1, PTPN11, RB1, SMAD4, SMARCB1, STK11. For example, CDH1, ERBB4, FBXW7, HNF1A, JAK3, NPM1, PTPN11, RB1, SMAD4, SMARCB1, STK11 may be assessed in addition to the biomarkers above. In an embodiment, the molecular profile comprises mutational analysis of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 of ABL1, AKT1, ALK, APC, ATM, BRAF, CDH1, cKIT, cMET, CSF1R, CTNNB1, EGFR, ERBB2, ERBB4, FBXW7, FGFR1, FGFR2, FLT3, GNA11, GNAQ, GNAS, HNF1A, HRAS, IDH1, JAK2, JAK3, KDR (VEGFR2), KRAS, MLH1, MPL, NOTCH1, NPM1, NRAS, PDGFRA, PIK3CA, PTEN, PTPN11, RB1, RET, SMAD4, SMARCB1, SMO, STK11, TP53, VHL. For example, the molecular profile may comprise or consist of mutational analysis of ABL1, AKT1, ALK, APC, ATM, BRAF, CDH1, eKIT, cMET, CSF1R, CTNNB1, EGFR, ERBB2, ERBB4, FBXW7, FGFR1, FGFR2, FLT3, GNA11, GNAQ, GNAS, HNF1A, HRAS, IDH1, JAK2, JAK3, KDR (VEGFR2), KRAS, MLH1, MPL, NOTCH1, NPM1, NRAS, PDGFRA, PIK3CA, PTEN, PTPN11, RB1, RET, SMAD4, SMARCB1, SMO, STK11, TP53, VHL.

In still other embodiments, the molecular profile comprises mutational analysis of 1, 2, 3, 4, 5, 6, 7, 8, 9, 10, 11, 12, 13, 14, 15, 16, 17, 18, 19 or 20 of ALK, BRAF, BRCA1, BRCA2, EGFR, ERRB2, GNA11, GNAQ, IDH1, IDH2, KIT, KRAS, MET, NRAS, PDGFRA, PIK3CA, PTEN, RET, SRC, TP53. The molecular profile may comprise mutational analysis of 1, 2, 3, 4, 5, 6, 7, 8, 9, 10, 11, 12, 13, 14, 15, 16, 17, 18, 19, 20, 21, 22, 23, 24, 25, 26, 27 or 28 of AKT1, HRAS, GNAS, MEK1, MEK2, ERK1, ERK2, ERBB3, CDKN2A, PDGFRB, IFG1R, FGFR1, FGFR2, FGFR3, ERBB4, SMO, DDR2, GRB1, PTCH, SHH, PD1, UGT1A1, BIM, ESR1, MLL, AR, CDK4, SMAD4. The molecular profile may also comprise mutational analysis of 1, 2, 3, 4, 5, 6, 7, 8, 9, 10, 11, 12, 13, 14, 15, 16, 17, 18, 19, 20 or 21 of ABL, APC, ATM, CDH1, CSFR1, CTNNB1, FBXW7, FLT3, HNF1A, JAK2, JAK3, KDR, MLH1, MPL, NOTCH1, NPM1, PTPN11, RB1, SMARCB1, STK11, VHL. The genes assessed by mutational analysis may comprise at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, 100, 110, 120, 130, 140, 150, 160, 170, 180, 190, at least 200 genes, or all genes, selected from the group consisting of ABL1, AKT1, AKT2, AKT3, ALK, APC, AR, ARAF, ARFRP1, ARID1A, ARID2, ASXL1, ATM, ATR, ATRX, AURKA, AURKB, AXL, BAP1, BARD1, BCL2, BCL2L2, BCL6, BCOR, BCORL1, BLM, BRAF, BRCA1, BRCA2, BRIP1, BTK, CARD11, CBFB, CBL, CCND1, CCND2, CCND3, CCNE1, CD79A, CD79B, CDC73, CDH1, CDK12, CDK4, CDK6, CDK8, CDKN1B, CDKN2A, CDKN2B, CDKN2C, CEBPA, CHEK1, CHEK2, CIC, CREBBP, CRKL, CRLF2, CSF1R, CTCF, CTNNA1, CTNNB1, DAXX, DDR2, DNMT3A, DOT1L, EGFR, EMSY (Cllorf30), EP300, EPHA3, EPHAS, EPHB1, ERBB2, ERBB3, ERBB4, ERG, ESR1, EZH2, FAM123B (WTX), FAM46C, FANCA, FANCC, FANCD2, FANCE, FANCF, FANCG, FANCL, FBXW7, FGF10, FGF14, FGF19, FGF23, FGF3, FGF4, FGF6, FGFR1, FGFR2, FGFR3, FGFR4, FLT1, FLT3, FLT4, FOXL2, GATA1, GATA2, GATA3, GID4 (C17orf39), GNA11, GNA13, GNAQ, GNAS, GPR124, GRIN2A, GSK3B, HGF, HRAS, IDH1, IDH2, IGF1R, IKBKE, IKZF1, IL7R, INHBA, IRF4, IRS2, JAK1, JAK2, JAK3, JUN, KAT6A (MYST3), KDMSA, KDMSC, KDM6A, KDR, KEAP1, KIT, KLHL6, KRAS, LRP1B, MAP2K1, MAP2K2, MAP2K4, MAP3K1, MCL1, MDM2, MDM4, MED12, MEF2B, MEN1, MET, MITF, MLH1, MLL, MLL2, MPL, MRE11A, MSH2, MSH6, MTOR, MUTYH, MYC, MYCL1, MYCN, MYD88, NF1, NF2, NFE2L2, NFKBIA, NKX2-1, NOTCH1, NOTCH2, NPM1, NRAS, NTRK1, NTRK2, NTRK3, NUP93, PAK3, PALB2, PAXS, PBRM1, PDGFRA, PDGFRB, PDK1, PIK3CA, PIK3CG, PIK3R1, PIK3R2, PPP2R1A, PRDM1, PRKAR1A, PRKDC, PTCH1, PTEN, PTPN11, RAD50, RAD51, RAF1, RARA, RB1, RET, RICTOR, RNF43, RPTOR, RUNX1, SETD2, SF3B1, SMAD2, SMAD4, SMARCA4, SMARCB1, SMO, SOCS1, SOX10, SOX2, SPEN, SPOP, SRC, STAG2, STAT4, STK11, SUFU, TET2, TGFBR2, TNFAIP3, TNFRSF14, TOP1, TP53, TSC1, TSC2, TSHR, VHL, WISP3, WT1, XPO1, ZNF217, ZNF703. The mutational analysis may be performed to detect a gene rearrangement, e.g., a rearrangement in 1, 2, 3, 4, 5, 6, 7, 8, 9, 10, 11, 12, 13, 14, 15, 16, 17, 18 or 19 of ALK, BCR, BCL2, BRAF, EGFR, ETV1, ETV4, ETV5, ETV6, EWSR1, MLL, MYC, NTRK1, PDGFRA, RAF1, RARA, RET, ROS1, TMPRSS2.

Molecular Profiling with Prioritized Sequencing (4.6, 4.7)

The invention further provides molecular profiles that use IHC for expression profiling and Next Generation sequencing for mutational analysis. Such profiles are described in FIGS. 35A-I and Table 26. The profiling is performed using the rules for the biomarker—drug associations for the various cancer lineages as described for FIGS. 33A-Q and Tables 7-24 above. An expanded set of genes may be assessed by mutational analysis for each molecular profile, as described further below.

Table 26 presents a view of the information that is reported for the molecular profiles. Modifications made dependent on cancer lineage are indicated in the table. The columns headed “Agent/Biomarker Status Reported” provide either candidate agents (e.g., drugs) or biomarker status to be included in the report. Where agents are indicated, the association of the agent with the indicated biomarker is included in the report. Where a status is indicated (e.g., mutational status, protein expression status, gene copy number status), the biomarker status is indicated in the report instead of drug associations. The candidate agents may comprise those undergoing clinical trials, as indicated.

TABLE 26
Molecular Profile and Report Parameters

| Agent(s)/Biomarker Status | | |
|---|---|---|
| Reported | Biomarker | Platform |
| docetaxel, paclitaxel, nab- | Pgp | IHC |
| paclitaxel, protein expression | SPARCm | IHC |
| | SPARCp | IHC |
| | TLE3 | IHC |
| | TUBB3 | IHC |
| capecitabine, fluorouracil, | TS | IHC |
| pemetrexed | | |
| doxorubicin, liposomal- | HER2 | FISH/CISH |
| doxorubicin, epirubicin, | TOP2A | IHC (excluding Breast) |
| protein expression | | FISH/CISH (Breast only) |
| | Pgp | IHC |
| irinotecan, topotecan | TOPO1 | IHC |
| gemcitabine | RRM1 | IHC |
| imatinib | cKIT | NextGen Sequencing |
| | PDGFRA | NextGen Sequencing |
| temozolomide, dacarbazine† | MGMT (excluding Glioma) | IHC (excluding Glioma) |
| | MGMT-Me (Glioma ONLY) | Pyrosequencing (Glioma ONLY) |
| | IDH1 | NextGen Sequencing |
| | (assoc. in High Grade | |
| | Glioma only) | |
| vandetanib | RET | NextGen Sequencing |
| abiraterone, bicalutamide, | AR | IHC |
| flutamide, protein expression | | |
| anastrozole, exemestane, | ER | IHC |
| falvestrant, goserelin, | PR | IHC |
| megestrol acetate, letrozole, | | |
| leuprolide, tamoxifen, | | |
| toremifene, protein | | |
| expression | | |
| trastuzumab | HER2 | IHC; FISH/CISH |
| | PTEN (assoc. in Breast only) | IHC |
| | PIK3CA (assoc. in Breast only) | NextGen Sequencing |
| lapatinib, pertuzumab, T- | HER2 | IHC, FISH/CISH |
| DM1, clinical trials | | |
| everolimus, temsirolimus, | ER (assoc. in Breast only) | IHC |
| clinical trials | HER2 (assoc. in Breast only) | IHC; FISH/CISH |
| | PIK3CA | NextGen Sequencing |
| cetuximab, panitumumab† | BRAF | NextGen Sequencing |
| (assoc. in CRC only) | KRAS | NextGen Sequencing |
| | NRAS | NextGen Sequencing |
| | PIK3CA | NextGen Sequencing |
| | PTEN | IHC |
| cetuximab† (assoc. in | EGFR (NSCLC only) | IHC (H-score) |
| NSCLC only) | | (NSCLC only) |
| erlotinib, gefitinib† | EGFR (NSCLC only) | NextGen Sequencing |
| (assoc. in NSCLC only) | | (NSCLC only) |
| | KRAS | NextGen Sequencing |
| | PIK3CA | NextGen Sequencing |
| | cMET | FISH/CISH |
| | PTEN | IHC |
| crizotinib† | ALK (assoc. in NSCLC only) | FISH |
| | | (NSCLC only) |
| | ROS1 (assoc. in NSCLC only) | |
| vemurafenib† (assoc. in | BRAF | NextGen Sequencing |
| Melanoma and Uveal | | PCR (cobas ®) |
| Melanoma only) | | |
| dabrafenib†, trametinib*† | BRAF | NextGen Sequencing |
| (assoc. in Melanoma only) | | PCR (cobas ®) |
| sunitinib† (assoc. in GIST | cKIT | NextGen Sequencing |
| only) | | |
| clinical trials† (HDAC and | GNA11 (assoc. in Uveal Melanoma only) | NextGen Sequencing |
| MEK inhibitors) | | (Uveal Melanoma only) |
| (assoc. in Uveal Melanoma | | |
| only) | | |
| clinical trials (cMET | cMET | IHC, FISH/CISH |
| inhibitors) | | |
| clinical trials (MEK and | BRAF | NextGen Sequencing |
| BRAF inhibitors) | KRAS | NextGen Sequencing |
| | NRAS | NextGen Sequencing |
| clinical trials (angiogenesis | VHL | NextGen Sequencing |
| inhibitors) | | |
| clinical trials (PIK3CA, | PTEN | NextGen Sequencing |
| mTOR, MEK, angiogenesis, | | |
| and IGF pathway inhibitors) | | |

†Assay and therapy will only be performed and reported for specific tumor types.
*Trametinib association will include BRAF by Next-Generation Sequencing testing for V600K mutations.

The molecular profile in Table 26 can be used to profile any cancer for selected a candidate treatment, e.g., by assessing a solid tumor sample as described herein. The biomarkers used for associations with specific cancer lineages are indicated in Table 26. FIGS. 35A-I further illustrate lineage specific profiling that can be performed. FIG. 35A illustrates a molecular profile for any solid tumor. FIG. 35B illustrates a molecular profile for an ovarian cancer. FIG. 35C illustrates a molecular profile for a melanoma. FIG. 35D illustrates a molecular profile for a uveal melanoma. FIG. 35E illustrates a molecular profile for a non-small cell lung cancer (NSCLC). FIG. 35F illustrates a molecular profile for a breast cancer. FIG. 35G illustrates a molecular profile for a colorectal cancer (CRC). FIG. 35H illustrates a molecular profile for a glioma. FIG. 351 illustrates individual marker profiling that can be added to any of the molecular profiles in FIGS. 35A-35G. As described, each of the molecular profiles in FIGS. 35A-I and Table 26 can be performed in conjunction with expanded mutational analysis as described above. See, e.g., Table 25 and accompanying text.

### Sample-Dependent Molecular Profiling (4.2)

The molecular profiling that is performed may depend on the amount and quality of sample that is available. For example, certain molecular profiling techniques can be performed with lesser amount of quality sample than other techniques. Thus, in some aspects the invention provides a molecular profile wherein the techniques performed depend on the amount and/or quality of the sample. For example, RT-PCR can be used to measure gene expression if sufficient sample is available; otherwise, IHC is performed to measure protein expression of the same biomarker. Such substitution may require that the evidence is available to support the substitution in order for the alternatively biomarker to be used to assess the likely benefit or not of a candidate agent. Sample dependent molecular profiles are described in more detail in this Section.

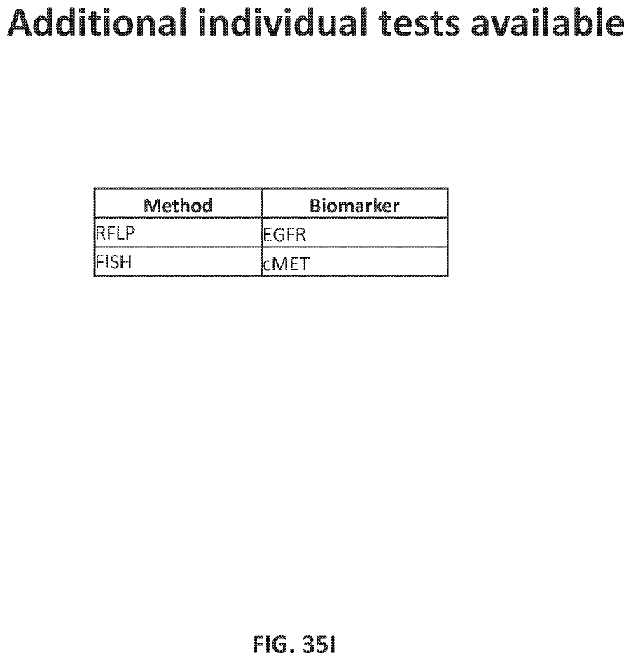

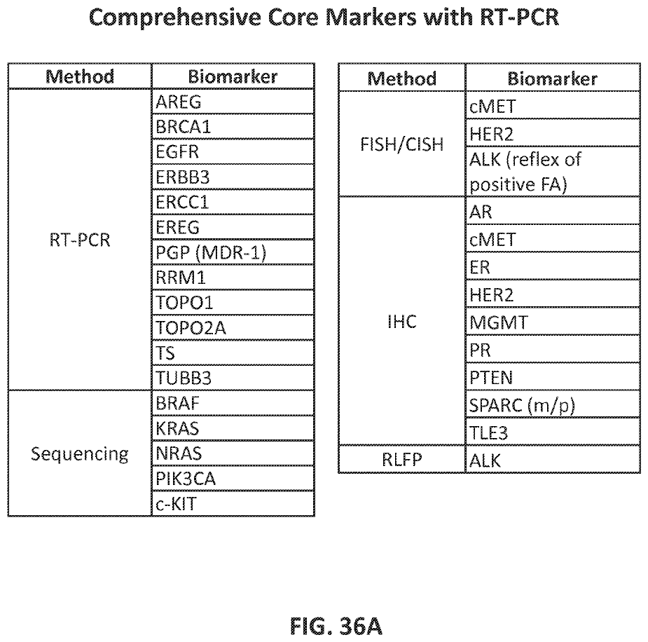

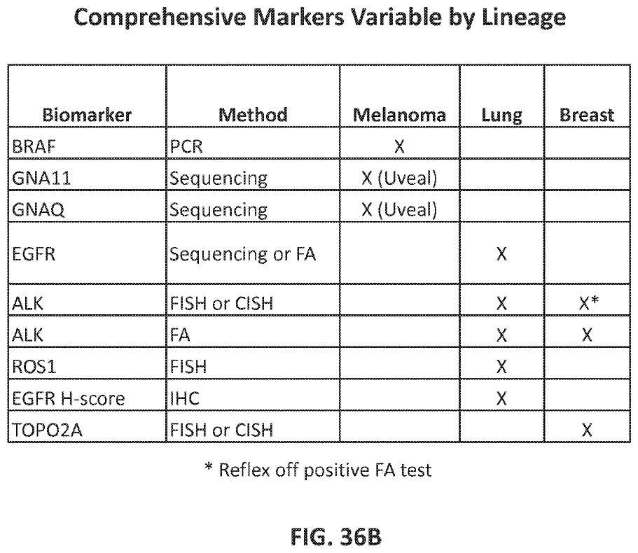

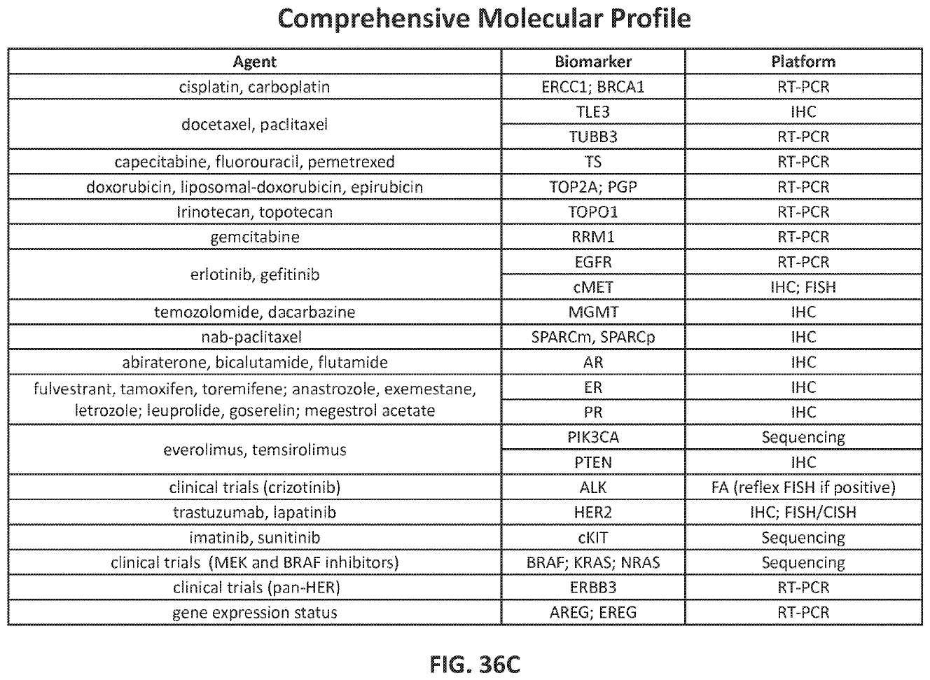

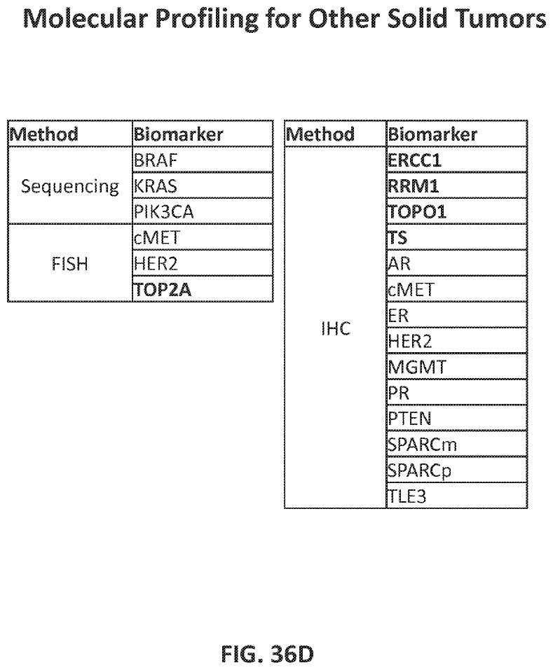

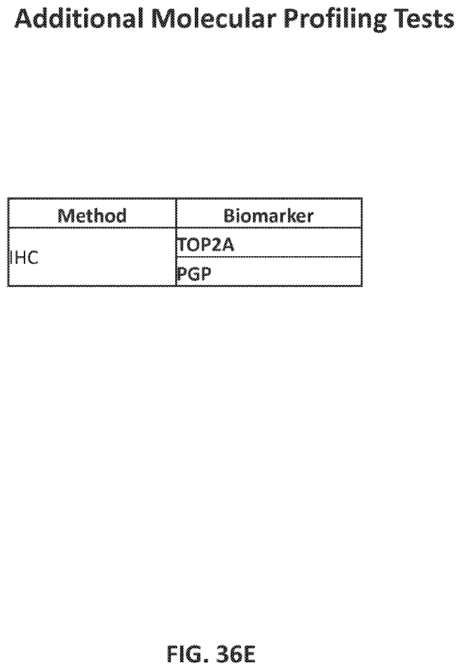

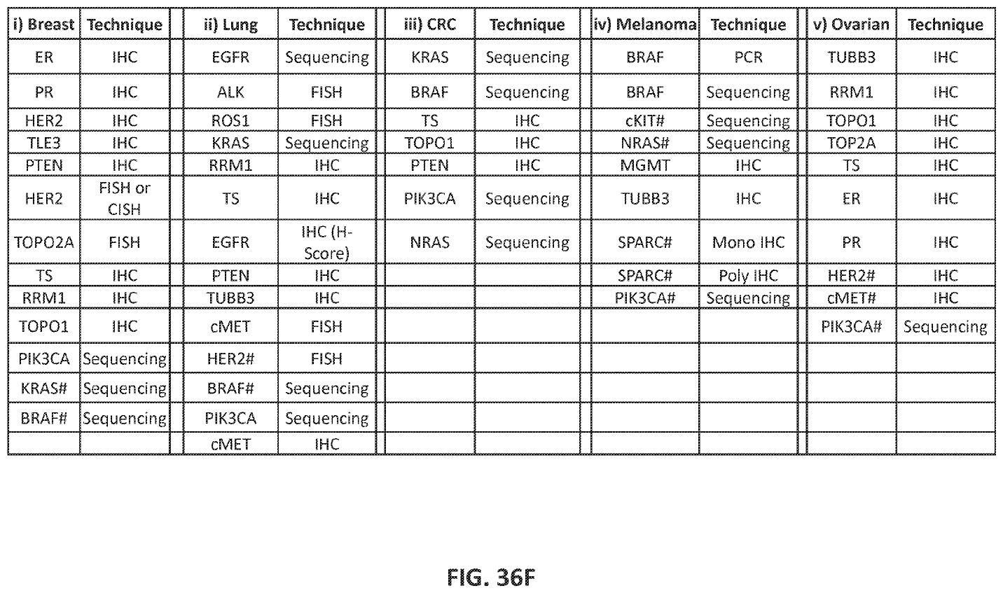

Consider an exemplary comprehensive molecular profile for any cancer comprising assessment of the biomarkers as illustrated in FIG. 36A and FIG. 36B in order to determine whether treatments in FIG. 36C are likely beneficial or not. The molecular profile uses RT-PCR to determine gene expression. As shown in FIG. 36A, the profiling may comprise: 1) RT-PCR to assess 1, 2, 3, 4, 5, 6, 7, 8, 9, 10, 11 or 12 of AREG, BRCA1, EGFR, ERBB3, ERCC1, EREG, PGP (MDR-1), RRM1, TOPO1, TOPO2A, TS, TUBB3; 2) sequencing to assess 1, 2, 3, 4 or 5 of BRAF, c-KIT, KRAS, NRAS, PIK3CA; 3) ISH to assess 1, 2, or 3 of ALK, cMET, HER2; 4) IHC to assess 1, 2, 3, 4, 5, 6, 7, 8, 9 or 10 of AR, cMET, ER, HER2, MGMT, PR, PTEN, SPARC (m/p), TLE3; and/or fragment analysis (e.g., RFLP) to assess ALK. As shown in FIG. 36B, certain additional biomarkers are assessed depending on tumor lineage, including: 1) BRAF by PCR (e.g., cobas PCR) and/or sequencing of GNAQ and/or GNA11 for melanoma; 2) sequencing or fragment analysis of EGFR, ISH analysis of ROS1, and/or IHC H-score analysis of EGFR for lung cancer; and 3) ISH analysis of TOPO2A for breast cancer. The biomarker-treatment associations for this molecular profile may comprise those associations in FIG. 36C and determination of likely benefit or not of the treatments based on the profiling results can be according to the rules in Table 27. Table 27 indicates whether the indicated markers are profiled for gastrointestinal stromal tumor (GIST) and/or profiling of any cancer. See column headed “GIST, Comprehensive, or Both.” The class of drug and illustrative drugs of the indicated class are indicated in the columns “Class of Drugs” and “Drugs,” respectively. The columns headed “Biomarker Result” illustrate illustrative methods of profiling the indicated biomarkers, generally as true (“T”) or false (“F”) or any. One of skill will appreciate that alternative methods can be used to analyze the biomarkers as appropriate. For example, expression analysis performed by RT-PCR could be performed by microarray or other expression analysis method such as those described herein or known in the art. The joint result of the indicated biomarker results combined to predict a benefit or not of the indicated candidate drugs. As an example of the logic used to select a drug treatment in Table 27, consider the first rules concerning ERCC1 and BRCA1 to assess the efficacy of platinum compounds. If gene expression of ERCC1 is found to be low by RT-PCR (ERCC1 low=T), then platinum compounds are predicted to have treatment benefit (T). However, if low expression of ERCC1 is determined to be false, then the expression of BRCA1 will determine the expected benefit with platinum compounds: if expression of ERCC1 is not low (i.e., ERCC1 low=F) and expression of BRCA1 is low (i.e., BRCA1 low=T), then platinum compounds are expected to be of benefit (i.e., overall benefit=T); if expression of ERCC1 is not low (i.e., ERCC1 low=F) and expression of BRCA1 is not low or is not determined (i.e., BRCA1 low=F or No Data), then platinum compounds are not expected to be of benefit (i.e., overall benefit=F).

The molecular profile for GIST can comprise a comprehensive profile with the additional molecular profiling indicated for a GIST in Table 27, namely differential sequence analysis of cKIT in GIST versus other cancers to predict treatment benefit with tyrosine kinase inhibitors (TKI). In GIST, imatinib associates with mutations in exons 9, 11 and/or 13 of cKIT, sunitinib associates with mutations in exon 9 of cKIT, and sorafenib associates with mutations in exons 9 and/or 11 of cKIT. In all other lineages, imatinib and sunitinib associate with mutations in exon 11 and/or 13 of cKIT.

TABLE 27
Comprehensive Molecular Profile using RT-PCR

| GIST, | | | | | | |
|---|---|---|---|---|---|---|
| Comprehensive, | Class of | | Biomarker | Biomarker | Biomarker | Treatment |
| or Both | Drugs | Drugs | Result | Result | Result | Benefit |
| | | cisplatin, | | | | |
| | Platinum | carboplatin, | ERCC1 Low | BRCA1 Low | | Overall |
| Both | compounds | oxaliplatin | (RT-PCR) | (RT-PCR) | | Benefit |
| | | | T | Any | | T |
| | | | F | Any | | F |
| | | | No Data | T | | T |
| | | | No Data | F | | F |
| | | | No Data | No Data | | Indeterminate |
| | Anthracyclines | doxorubicin, | | | | |
| | and | liposomal- | | | | |
| | related | doxorubicin, | TOP2A High | PGP Low | | Overall |
| Both | substances | epirubicin | (RT-PCR) | (RT-PCR) | | Benefit |
| | | | T | T or No Data | | T |
| | | | T | F | | F |
| | | | F | Any | | F |
| | | | No Data | T | | T |
| | | | No Data | F | | F |
| | | | No Data | No Data | | Indeterminate |
| | | | TLE3 | | | |
| | | docetaxel, | Positive | TUBB3 Low | | Overall |
| Both | Taxanes | paclitaxel | (IHC) | (RT-PCR) | | Benefit |
| | | | T | Any | | T |
| | | | F | Any | | F |
| | | | No Data | T | | T |
| | | | No Data | F | | F |
| | | | No Data | No Data | | Indeterminate |
| | | | SPARC | SPARC | | |
| | | | MONO | POLY | | |
| | | nab- | Positive | Positive | | Overall |
| Both | Taxanes | paclitaxel | (IHC) | (IHC) | | Benefit |
| | | | T | Any | | T |
| | | | F | T | | T |
| | | | F | F or No Data | | F |
| | | | No Data | T | | T |
| | | | No Data | F | | F |
| | | | No Data | No Data | | Indeterminate |
| | | | RRM1 Low | | | Overall |
| Both | Antimetabolites | gemcitabine | (RT-PCR) | | | benefit |
| | | | T | | | T |
| | | | F | | | F |
| | | | No Data | | | Indeterminate |
| | | pemetrexed, | | | | |
| | Fluoropyrimidines/ | fluorouracil, | TS Low (RT- | | | Overall |
| Both | Antimetabolites | capecitabine | PCR) | | | benefit |
| | | | T | | | T |
| | | | F | | | F |
| | | | No Data | | | Indeterminate |
| | | | TOPO1 | | | |
| | TOPO1 | irinotecan, | High (RT- | | | Overall |
| Both | inhibitors | topotecan | PCR) | | | benefit |
| | | | T | | | T |
| | | | F | | | F |
| | | | No Data | | | Indeterminate |
| | | | MGMT | | | |
| | Alkylating | temozolomide, | Negative | | | Overall |
| Both | agents | dacarbazine | (IHC) | | | benefit |
| | | | T | | | T |
| | | | F | | | F |
| | | | No Data | | | Indeterminate |
| | | | PIK3CA | PTEN | | |
| | mTOR | everolimus, | Mutated | Negative | | Overall |
| Both | inhibitors | temsirolimus | (Sequencing) | (IHC) | | benefit |
| | | | T | Any | | T |
| | | | F | T | | T |
| | | | F | F | | F |
| | | | F | No Data | | Indeterminate |
| | | | No Data | T | | T |
| | | | No Data | F or No Data | | Indeterminate |
| | | bicalutamide, | | | | |
| | Anti- | flutamide, | AR Positive | | | Overall |
| Both | androgens | abiraterone | (IHC) | | | Benefit |
| | | | T | | | T |
| | | | F | | | F |
| | | | No Data | | | Indeterminate |
| | | tamoxifen, | | | | |
| | Anti- | toremifene, | ER Positive | PR Positive | | Overall |
| Both | estrogens | fulvestrant | (IHC) | (IHC) | | Benefit |
| | | | T | Any | | T |
| | | | F | T | | T |
| | | | F | F | | F |
| | | | F | No Data | | Indeterminate |
| | | | No Data | T | | T |
| | | | No Data | F or No Data | | Indeterminate |
| | Endocrine | | | | | |
| | therapy- | letrozole, | | | | |
| | enzyme | anastrozole, | ER Positive | PR Positive | | Overall |
| Both | inhibitor | exemestane | (IHC) | (IHC) | | Benefit |
| | | | T | Any | | T |
| | | | F | T | | T |
| | | | F | F | | F |
| | | | F | No Data | | Indeterminate |
| | | | No Data | T | | T |
| | | | No Data | F or No Data | | Indeterminate |
| | | medroxy- | | | | |
| | | progesterone, | | | | |
| | | megestrol | ER Positive | PR Positive | | Overall |
| Both | Progestogens | acetate | (IHC) | (IHC) | | Benefit |
| | | | T | Any | | T |
| | | | F | T | | T |
| | | | F | F | | F |
| | | | F | No Data | | Indeterminate |
| | | | No Data | T | | T |
| | | | No Data | F or No Data | | Indeterminate |
| | Gonadotropin | | | | | |
| | releasing | | | | | |
| | hormone | leuprolide, | ER Positive | PR Positive | | Overall |
| Both | analogs | goserelin | (IHC) | (IHC) | | Benefit |
| | | | T | Any | | T |
| | | | F | T | | T |
| | | | F | F | | F |
| | | | F | No Data | | Indeterminate |
| | | | No Data | T | | T |
| | | | No Data | F or No Data | | Indeterminate |
| | | | HER2 | HER2 | | |
| | | | Positive | Amplified | | Overall |
| Both | TKI | lapatinib | (IHC) | (FISH) | | Benefit |
| | | | T | Any | | T |
| | | | F | T or | | T |
| | | | | Equivocal | | |
| | | | | High | | |
| | | | F | For | | F |
| | | | | Equivocal | | |
| | | | | Low | | |
| | | | F | No Data | | Indeterminate |
| | | | Equivocal | T or | | T |
| | | | | Equivocal | | |
| | | | | High | | |
| | | | Equivocal | F or | | F |
| | | | | Equivocal | | |
| | | | | Low | | |
| | | | Equivocal | No Data | | Indeterminate |
| | | | No Data | T or | | T |
| | | | | Equivocal | | |
| | | | | High | | |
| | | | No Data | F, Equivocal | | Indeterminate |
| | | | | Low or No | | |
| | | | | Data | | |
| | Monoclonal | | | | | |
| | antibodies | | | | | |
| | (Her2- | | HER2 | HER2 | | |
| | targeted- | | Positive | Amplified | | Overall |
| Both | trastuzuma) | trastuzumab | (IHC) | (FISH) | | Benefit |
| | | | T | Any | | T |
| | | | F | T or | | T |
| | | | | Equivocal | | |
| | | | | High | | |
| | | | F | F or | | F |
| | | | | Equivocal | | |
| | | | | Low | | |
| | | | F | No Data | | Indeterminate |
| | | | Equivocal | T or | | T |
| | | | | Equivocal | | |
| | | | | High | | |
| | | | Equivocal | F or | | F |
| | | | | Equivocal | | |
| | | | | Low | | |
| | | | Equivocal | No Data | | Indeterminate |
| | | | No Data | T or | | T |
| | | | | Equivocal | | |
| | | | | High | | |
| | | | No Data | F, Equivocal | | Indeterminate |
| | | | | Low or No | | |
| | | | | Data | | |
| | | | | cMET | cMET | |
| | | erlotinib, | EGFR High | Positive | Amplified | Overall |
| Both | TKI | gefitinib | (RT-PCR) | (IHC) | (FISH) | Benefit |
| | | | T | Any | Any | T |
| | | | F | Any | Any | F |
| | | | No Data | Any | Any | Indeterminate |
| | | | ALK | | | |
| | | | Positive | ALK | | Overall |
| Both | TKI | crizotinib | (FISH) | Positive (FA) | | Benefit |
| | | | T | Any | | T |
| | | | F | Any | | F |
| | | | No Data | Any | | Indeterminate |
| | | | c-KIT | | | |
| | | | Mutated | | | Overall |
| GIST | TKI | imatinib | (Sequencing) | | | Benefit |
| | | | T | | | T |
| | | | F | | | F |
| | | | No Data | | | Indeterminate |
| | | | c-KIT | | | |
| | | | Mutated | | | Overall |
| GIST | TKI | sunitinib | (Sequencing) | | | Benefit |
| | | | T | | | T |
| | | | F | | | F |
| | | | No Data | | | Indeterminate |
| | | | c-KIT | | | |
| | | | Mutated | | | Overall |
| GIST | TKI | sorafenib | (Sequencing) | | | Benefit |
| | | | T | | | T |
| | | | F | | | F |
| | | | No Data | | | Indeterminate |
| | | | c-KIT | | | |
| | | imatinib, | Mutated | | | Overall |
| Comprehensive | TKI | sunitinib | (Sequencing) | | | Benefit |
| | | | T | | | T |
| | | | F | | | F |
| | | | No Data | | | Indeterminate |

In an embodiment, the invention provides a comprehensive molecular profile for cancer comprising one or more, e.g., 1, 2, 3, 4, 5, 6, 7, 8, 9, 10, 11, 12, 13, 14, 15, 16, 17, 18, 19, 20, 21, 22, 23, 24, 25, 26 or 27 of: ALK, AR, AREG, BRAF, BRCA1, c-KIT, cMET, EGFR, ER, ERBB3, ERCC1, EREG, HER2, KRAS, MGMT, NRAS, PGP (MDR-1), PIK3CA, PR, PTEN, RRM1, SPARC, TLE3, TOPO1, TOPO2A, TS, TUBB3. The invention further provides a method of selecting a candidate treatment for a cancer comprising assessment of one or more members of the comprehensive cancer profile using one or more molecular profiling method presented herein, e.g., FISH/CISH, IHC, RT-PCR, expression array, sequencing, FA such as RFLP, etc. In one embodiment, FISH/CISH is used to assess one or more, e.g., 1 or 2, of: cMET and HER2. In an embodiment, protein analysis such as IHC is used to assess one or more, e.g., 1, 2, 3, 4, 5, 6, 7, 8 or 9, of: AR, cMET, ER, HER2, MGMT, PR, PTEN, SPARC, TLE3. The IHC can be used to ascertain an IHC score (H-score), which takes into account the percentage of cells (0-100%) as well as each staining intensity category (0-3+) to compute a semi-quantitative score between 0 and 300. In another embodiment, expression analysis, e.g., by RT-PCR (qPCR) or microarray, is used to assess one or more of, e.g., 1, 2, 3, 4, 5, 6, 7, 8, 9, 10, 11, or 12, of: AREG, BRCA1, EGFR, ERBB3, ERCC1, EREG, PGP (MDR-1), RRM1, TOPO1, TOPO2A, TS, TUBB3. In still another embodiment, sequence analysis is used to assess one or more, e.g., 1, 2, 3, 4 or 5, of: BRAF, KRAS, NRAS, PIK3CA, c-KIT. The comprehensive cancer profile can also comprise assessment of the presence of ALK or an ALK mutation/translocation/rearrangement, e.g., an EML4-ALK fusion, e.g., by FISH, RT-PCR, sequencing or fragment analysis (FA). In an embodiment, the molecular profile further comprises detection of the presense of VEGFR2, e.g., by RT-PCR. Any biomarker disclosed herein, e.g., in Table 2, Table 6 or Table 25, can be assessed as part of the comprehensive molecular profile. The comprehensive profile for a malignancy of any lineage can be as shown in FIGS. 36A-C. The profile can be used to identify drugs as likely beneficial or not based on rules in Table 27.

The comprehensive profile can further comprise molecular profiling of certain genes in the context of specific cancer lineage. For example, the comprehensive profile can comprise the molecular profiling described above and in addition one or more of the following markers. A comprehensive profile of melanoma can include molecular profiling of BRAF, GNA11 and/or GNAQ. For example, one or more of these biomarkers can be assessed for a mutation, e.g., by sequencing or PCR. In an embodiment, BRAF is assessed using the FDA approved Cobas® 4800 BRAF V600 Mutation Test from Roche Molecular Diagnostics (Roche Diagnostics, Indianapolis, Ind.). According to the manufacturer, the kit comprises a real-time PCR test to detect the BRAF V600E (1799 T>A) mutation in human melanoma, e.g., in formalin-fixed, paraffin-embedded (FFPE) tissue. It is designed to help select patients for treatment with vemurafenib, an oral medicine designed to treat patients whose melanoma tumors harbor a mutated form of the BRAF gene. The test may also detect other V600 mutations such as V600D and V600K. Vemurafenib is designed to target and inhibit some mutated forms of the BRAF protein found in about half of all cases of melanoma. GNAQ/GNA11 mutations can promote tumor growth and metastatis. MEK inhibitors may inhibit the GNAQ/GNA11 pathway. Similarly, a comprehensive profile of non-small cell lung cancer can include additional molecular profiling of EGFR and/or ALK. For example, and EGFR mutation can be detected by sequence analysis and/or fragment analysis. EGFR protein can be assessed by IHC, including by determining an H-score. ALK can be assessed using FISH and/or CISH. In an embodiment, ALK is assessed using the Vysis ALK Break Apart FISH Probe Kit from Abbott Molecular, Inc. (Des Plaines, Ill.). According to the manufacturer, this kit comprises a laboratory test that uses DNA probes with attached fluorescent dyes to detect the presence of chromosomal rearrangements of the ALK gene, located on chromosome 2, in a non-small cell lung cancer (NSCLC) tissue sample. If the test result indicates the presence of rearrangements (such as translocation) involving the ALK gene in the cancer cell, then a patient with NSCLC may be eligible for treatment with the cancer drug crizotinib. Crizotinib selectively interferes with the ALK gene and can benefit patients with ALK mutations. In addition, the comprehensive profile for a breast cancer can comprise further molecular profiling of TOPO2A, e.g., using FISH or CISH. In sum, embodiments of the comprehensive profile can be as shown in FIGS. 36A-36C with rules to identify drugs as likely beneficial or not based as shown in Table 27.

The molecular profiles of the invention can comprise further gene and gene products to identify additional biomarker-treatment associations. In an embodiment, the molecular profile comprises one or more additional gene or gene product listed in Table 2, Table 6 or Table 25. For example, the molecular profile may comprise one or more additional gene or gene product selected from the group consisting of MSH2, ERBB4, ROS1, MGMT, and a combination thereof. Any appropriate technique can be used to assess the gene and/or gene products. In a non-limiting example, the molecular profile can include one or more additional analysis selected from the group consisting of allele-specific PCR for BRAF and/or KRAS; RT-PCR for one or more of ER, HER2, MSH2 and PR; sequence analysis for ERBB4; FISH, fragment analysis and/or microsatellite instability for ROS1 rearrangements and/or HER2 exon 20 insertion; pyrosequencing for MGMT methylation status; and a combination thereof.

As noted above, different technologies used for molecular profiles can require different amounts of the input biological sample. In some embodiments of the invention, the precise technology used depends upon the amount of tumor sample that is available. A threshold amount of tumor sample can be set to perform certain tests. For example, a threshold amount of tumor can be set for determining whether or not to perform RT-PCR for gene expression analysis. If insufficient tumor sample is available, then another technique for measuring expression levels can be performed, such as IHC to measure protein expression. Alternately, if there is not enough sample to perform RT-PCR, then FISH is performed. As another example, a threshold amount of tumor can be set for determining whether or not to perform Sanger sequence analysis. If insufficient tumor sample is available, then another technique for detecting a gene mutation can be performed, such as fragment analysis (FA). The threshold can depend on factors such as molecular profiling technique to be performed, size of the tumor sample, and percentage of tumor in the sample. In some embodiments, the patient sample is subjected to microdissection to select areas enriched in tumor before performing molecular profiling. Thus, the threshold can be set after microdissection as desired. In an embodiment, the threshold takes into account the size of the tumor sample available. The size required can be at least 0.1 mm², 0.5 mm², 1.0 mm², 1.5 mm², 2.0 mm², 2.5 mm², 3.0 mm², 3.5 mm², 4.0 mm², 4.5 mm², 5.0 mm², 6.0 mm², 7.0 mm², 8.0 mm², 9.0 mm², 10.0 mm², 11.0 mm², 12.0 mm², 13.0 mm², 14.0 mm², 15.0 mm², 16.0 mm², 17.0 mm², 18.0 mm², 19.0 mm², 20.0 mm², 22.5 mm², 25.0 mm², 27.5 mm², 30.0 mm², 32.5 mm², 35.0 mm², 37.5 mm², 40.0 mm², 45.0 mm², or at least 50.0 mm². In another embodiment, the threshold takes into account the percentage of tumor in the sample. The percentage of tumor required can be at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99%. The percentage can be expressed as the percentage of tumor nuclei. When the sample is cut into pathology slides, a minimum number of slides can be required. In still another embodiment, the threshold takes into account the number of sample slides available. The number of slides required can be at least 1, 2, 3, 4, 5, 6, 7, 8, 9, 10, 11, 12, 13, 14, 15, 16, 17, 18, 19, 20, 21, 22, 23, 24, 25, 30, 35, 40, 45, or at least 50 slides.

Any useful combination of parameters can be used to determine the threshold. For example, the threshold to determine whether to run RT-PCR or IHC/FISH may comprise having at least 1, 2, 3, 4, 5, 6, 7, 8, 9, 10, 11, 12, 13, 14, 15, 16, 17, 18, 19, 20, 21, 22, 23, 24, 25, 30, 35, 40, 45, or at least 50 slides pathology slides each having at least 0.1 mm², 0.5 mm², 1.0 mm², 1.5 mm², 2.0 mm², 2.5 mm², 3.0 mm², 3.5 mm², 4.0 mm², 4.5 mm², 5.0 mm², 6.0 mm², 7.0 mm², 8.0 mm², 9.0 mm², 10.0 mm², 11.0 mm², 12.0 mm², 13.0 mm², 14.0 mm², 15.0 mm², 16.0 mm², 17.0 mm², 18.0 mm², 19.0 mm², 20.0 mm², 22.5 mm², 25.0 mm², 27.5 mm², 30.0 mm², 32.5 mm², 35.0 mm², 37.5 mm², 40.0 mm², 45.0 mm², or at least 50.0 mm² of tumor sample with at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% tumor nuclei in a sample after microdissection.

In an embodiment, if sufficient tumor is available, RT-PCR is performed; otherwise, IHC or FISH are performed. For example, RT-PCR can be performed if the sample after microdissection comprises at least 2.0 mm², 2.5 mm², 3.0 mm², 3.5 mm², 4.0 mm², 4.5 mm², 5.0 mm², 6.0 mm², 7.0 mm², 8.0 mm², 9.0 mm², or 10.0 mm² of tumor and at least 50%, 55%, 60%, 65%, 70%, 75%, 80%, 85%, or 90% tumor nuclei; otherwise IHC or FISH is performed. At least 1, 2, 3, 4, 5, 6, 7, 8, 9, 10, 11, 12, 13, 14, 15, 16, 17, 18, 19, 20, 21, 22, 23, 24, 25, 30, 35, 40, 45, or at least 50 slides pathology slides can be required to perform RT-PCR. In an embodiment, RT-PCR is performed if the sample after microdissection comprises at least 15 slides having 5.0 mm² of tumor and at least 80% tumor nuclei; otherwise IHC or FISH is performed. The threshold can be applied to any biomarkers assessed by molecular profiling. For example, the threshold can be performed to determine whether to perform RT-PCR or IHC/FISH to assess one or more of, e.g., 1, 2, 3, 4, 5, 6, 7, 8, 9, 10, 11, or 12, of: AREG, BRCA1, EGFR, ERBB3, ERCC1, EREG, PGP (MDR-1), RRM1, TOPO1, TOPO2A, TS, and TUBB3. The threshold can be applied for any useful subset of these markers, including without limitation one or more of ERCC1, TS, TOPO1, TOP2A, RRM1 and PGP. In embodiments, if the threshold for performing RT-PCR is not met, IHC is performed for ERCC1, TS, TOPO1, RRM1 and PGP, and FISH is performed for TOP2A. If FISH is not possible, then IHC for both TOP2A and PGP may be performed instead.

In another embodiment, if sufficient tumor is available, nucleotide sequencing such as Sanger sequencing is performed; otherwise, fragment analysis such as RFLP is performed. For example, nucleotide sequencing can be performed if the sample after microdissection comprises at least 2.0 mm², 2.5 mm², 3.0 mm², 3.5 mm², 4.0 mm², 4.5 mm², 5.0 mm², 6.0 mm², 7.0 mm², 8.0 mm², 9.0 mm², or 10.0 mm² of tumor and at least 50%, 55%, 60%, 65%, 70%, 75%, 80%, 85%, or 90% tumor nuclei; otherwise fragment analysis is performed. In an embodiment, nucleotide sequencing is performed if the sample after microdissection comprises at least 50% tumor nuclei; otherwise fragment analysis is performed. The threshold can be applied to any biomarkers assessed by molecular profiling. For example, the threshold can be performed to determine whether to perform nucleotide sequencing or fragment analysis to assess one or more, e.g., 1, 2, 3, 4, 5 or 6, of: BRAF, KRAS, NRAS, PIK3CA, c-KIT, EGFR. The threshold can be applied for any useful subset of these markers, including without limitation EGFR.

In an aspect, the invention provides a method comprising microdissecting a tumor sample from a tissue sample, determining a size of the microdissected tumor sample and an amount of the microdissected sample that comprises tumor nuclei, and performing RT-PCR on the microdissected tumor sample to detect an amount of one or more biomarker target if the size of microdissected tumor sample is greater than or equal to 5.0 mm² and the microdissected tumor sample comprises 80% or more tumor nuclei, else performing IHC on the microdissected tumor sample to detect an amount of the one or more biomarker target. The one or more biomarker can be selected from the group consisting of ERCC1, TS, TOPO1, TOP2A, RRM1 and PGP. For example, the one or more biomarker can comprise ERCC1, TS, TOPO1, TOP2A, RRM1 and PGP. As noted above, the threshold size and percentage tumor nuclei can be adjusted as appropriate.

The comprehensive molecular profile in this Section (e.g., as shown in FIGS. 36A-C) can be adjusted to reflect such changes when the thresholds for running RT-PCR are not met. For example, if the sample after microdissection comprises at least 15 slides having 5.0 mm² of tumor and at least 80% tumor nuclei, then the molecular profiles shown in FIGS. 36A-C are used to guide selection of the candidate treatment. If the conditions for running RT-PCR are not met, then the alternate molecular profile shown in FIG. 36I) is used to guide selection of the candidate treatment/s. Biomarkers shown in bold in FIG. 36D indicate biomarkers whose molecular profiling technique was changed as the thresholds for RT-PCR were not met. Comparing then the molecular profiles shown in FIGS. 36A-C with the molecular profiles shown in FIG. 36D, it is observed that when the threshold for performing RT-PCR is not met, IHC is performed for ERCC1, TS, TOPO1, RRM1 and PGP, and FISH is performed for TOP2A. Furthermore, as shown in FIG. 36E, if FISH is not possible, then IHC for TOP2A and PGP may be performed instead.

The rules implemented for selection of the candidate treatment can be the same as those presented for RT-PCR, except that the expression results obtained using IHC are substituted. For example, overexpression observed with IHC can trigger the same rules as overexpression with RT-PCR and underexpression observed with IHC can trigger the same rules as underexpression with RT-PCR. With respect to the rules presented in Table 27, references to “Low (RT-PCR)” can be substituted with “Negative (IHC),” and references to “High (RT-PCR)” can be substituted with “Positive (IHC).” As a non-limiting example, associations between TOPO1 by RT-PCR and irinotecan can be substituted with associations between TOPO1 by IHC and irinotecan. Similarly, associations between ERCC1 by RT-PCR and platinum compounds can be substituted with associations between ERCC1 by IHC and platinum compounds. As still another example, associations between RRM1 by RT-PCR and gemcitabine can be substituted with associations between RRM1 by IHC and gemcitabine.

When the sample available is close to the threshold, multiple tests may be performed. For example, if any of the factors for performing RT-PCR or IHC/FISH are within 25% of the threshold value, e.g., 20%, 15%, 10%, 5%, both tests can be performed. In this case, the results of tests providing sufficient data will be applied to the rules above in order to select the candidate treatment. If both tests provide usable results a priority scheme can be used, e.g., when both RT-PCR and IHC are successfully performed on a sample. In an embodiment, results for IHC trump rules for RT-PCR in case of disagreement. Results for FISH can also trump rules for RT-PCR in this scenario. For example, IHC for any of TOPO1, TS, RRM1, TOPO2A, ERCC1, PGP can trump results of RT-PCR for TOPO1, TS, RRM1, TOPO2A, ERCC1, PGP, respectively. Inconsistent results can also depend on the particular biomarker-drug associations. In an embodiment, for TS and fluoropyrimidine rules, when TS PCR and IHC results are inconsistent, the overall benefit of fluoropyrimidine is deemed “Indeterminate.” In another embodiment, for RRM1 and gemcitabine rules, when RRM1 PCR and IHC results are inconsistent, the overall benefit of gemcitabine is deemed true when RRM1 PCR is low and false when RRM1 PCR is high. In still another embodiment, for TOPO1 rules, the benefit is “indeterminate” when Topo1 IHC does not provide results, regardless of whether the Topo1 RT-PCR has actionable data. When TOP2A FISH is used to replace TOP2A RT-PCR, when either TOP2A FISH or Her2 FISH show amplification, anthracyclines are considered to be of benefit.

As an alternative to, or in addition to, substituting laboratory techniques when lower amounts of sample are available, the invention contemplates that certain biomarker tests can be prioritized. FIG. 36F provides illustrative biomarker tests that can be prioritized for various lineages, e.g., when insufficient sample is available for comprehensive molecular profiling as provided herein (e.g., in FIGS. 33A-Q, 35A-I, 36A-E). The biomarkers can be prioritized by the strength of evidence of clinical utility and by standard of care practice guidelines, e.g., the NCCN compendia. Biomarkers followed by the symbol # in FIG. 36F indicate that the drug associated with that particular biomarker is not part of the NCCN compendia. FIG. 36Fi provides a priority panel for a breast cancer, wherein the panel comprises one or more of, e.g., 1, 2, 3, 4, 5, 6, 7, 8, 9, 10, 11, 12 or 13, of: ER assessed by IHC; PR assessed by IHC; HER2 assessed by IHC; TLE3 assessed by IHC; PTEN assessed by IHC; HER2 assessed by FISH or CISH; TOPO2A assessed by FISH; TS assessed by IHC; RRM1 assessed by IHC; TOPO1 assessed by IHC; PIK3CA assessed by Sequencing; KRAS assessed by Sequencing; and BRAF assessed by Sequencing. FIG. 36Fii provides a priority panel for a lung cancer, wherein the panel comprises one or more of, e.g., 1, 2, 3, 4, 5, 6, 7, 8, 9, 10, 11, 12, 13 or 14, of: EGFR assessed by Sequencing; ALK assessed by FISH; ROS1 assessed by FISH; KRAS assessed by Sequencing; RRM1 assessed by IHC; TS assessed by IHC; EGFR assessed by IHC (H-Score); PTEN assessed by IHC; TUBB3 assessed by IHC; cMET assessed by FISH; HER2 assessed by FISH; BRAF assessed by Sequencing; PIK3CA assessed by Sequencing; cMET assessed by IHC. FIG. 36Fiii provides a priority panel for a colorectal cancer (CRC), wherein the panel comprises one or more of, e.g., 1, 2, 3, 4, 5, 6 or 7, of: KRAS assessed by Sequencing; BRAF assessed by Sequencing; TS assessed by IHC; TOPO1 assessed by IHC; PTEN assessed by IHC; PIK3CA assessed by Sequencing; NRAS assessed by Sequencing. FIG. 36Fiv provides a priority panel for a melanoma, wherein the panel comprises one or more of, e.g., 1, 2, 3, 4, 5, 6, 7, 8 or 9, of: BRAF assessed by PCR; BRAF assessed by Sequencing; cKIT assessed by Sequencing; NRAS assessed by Sequencing; MGMT assessed by IHC; TUBB3 assessed by IHC; SPARC assessed by IHC using a monoclonal antibody; SPARC assessed by IHC using a polyclonal antibody; PIK3CA assessed by Sequencing. FIG. 36Fv provides a priority panel for a melanoma, wherein the panel comprises one or more of, e.g., 1, 2, 3, 4, 5, 6, 7, 8, 9, or 10 of: TUBB3 assessed by IHC; RRM1 assessed by IHC; TOPO1 assessed by IHC; TOP2A assessed by IHC; TS assessed by IHC; ER assessed by IHC; PR assessed by IHC; HER2 assessed by IHC; cMET assessed by IHC; PIK3CA assessed by Sequencing. The biomarkers assessed are linked to the likely benefit or lack of benefit of various chemotherapy agents using rules such as provided herein, e.g., in Tables 7-24 or 27. Priority panels can be constructed for other lineages also based on the available evidence.

### Clinical Trial Connector

Thousands of clinical trials for therapies are underway in the United States, with several hundred of these tied to biomarker status. In an embodiment, the molecular intelligence molecular profiles of the invention include molecular profiling of markers that are associated with ongoing clinical trials. Thus, the molecular profile can be linked to clinical trials of therapies that are correlated to a subject's biomarker profile. The method can further comprise identifying trial location(s) to facilitate patient enrollment. The database of ongoing clinical trials can be obtained from www.clinicaltrials.gov in the United States, or similar source in other locations. The molecular profiles generated by the methods of the invention can be linked to ongoing clinical trials and updated on a regular basis, e.g., daily, bi-weekly, weekly, monthly, or other appropriate time period.

Although significant advances in cancer treatment have been made in recent years, not all patients can be effectively treated within the standard of care paradigm. Many patients are eligible for clinical trials participation, yet less than 3 percent are actually enrolled in a trial, according to recent National Cancer Institute (NCI) statistics. The Clinical Trials Connector allows caregivers such as physicians to quickly identify and review global clinical trial opportunities in real-time that are molecularly targeted to each patient. In embodiments, the Clinical Trials Connector has one or more of the following features: Examines thousands of open and enrolling clinical trials; Individualizes clinical trials based on molecular profiling as described herein; Includes interactive and customizable trial search filters by: Biomarker, Mechanism of action, Therapy, Phase of study, and other clinical factors (age, sex, etc.). The Clinical Trials Connector can be a computer database that is accessed once molecular profiling results are available. In some embodiments, the database comprises the EmergingMed database (EmergingMed, New York, N.Y.).

Tables 7, 9, 11, 13, 15, 17 and 21 herein indicates an association of certain biomarkers in the molecular profiles of the invention with ongoing clinical trials. Profiling of the specified markers can provide an indication that a subject is a candidate for a clinical trial, e.g., by suggesting that an agent in a clinical trial may benefit the subject. For example, Table 7 indicates that molecular profiling of HER2, PIK3CA, PTEN, cMET and the other indicated gene mutations (i.e., as profiled using NGS) can associate ovarian cancer with ongoing clinical trials. Table 9 indicates that molecular profiling of HER2, ER/HER2/PIK3CA, AR, cMET and the other indicated gene mutations (i.e., as profiled using NGS) can associate breast cancer with ongoing clinical trials. Table 11 indicates that molecular profiling of PIK3CA, PTEN, cMET and the other indicated gene mutations (i.e., as profiled using NGS) can associate melanoma with ongoing clinical trials. Table 13 indicates that molecular profiling of PIK3CA, PTEN, cMET and the other indicated gene mutations (i.e., as profiled using NGS) can associate melanoma with ongoing clinical trials. Table 15 indicates that molecular profiling of cMET and the other indicated gene mutations (i.e., as profiled using NGS) can associate colorectal cancer with ongoing clinical trials. Table 17 indicates that molecular profiling of HER2, PIK3CA, cMET and the indicated gene mutations (i.e., as profiled using NGS) can associate NSCLC with ongoing clinical trials. Table 21 indicates that molecular profiling of HER2, PIK3CA, PTEN, cMET, EGFRvIII, IDH2 and the indicated gene mutations (i.e., as profiled using NGS) can associate various solid tumors with ongoing clinical trials. An illustrative listing of such clinical trials is found in Table 28 below.

FIG. 36C and Table 26 herein further indicate associations of certain biomarkers in the respective molecular profiles with ongoing clinical trials. The clinical trial connections are interpreted as indicated above.

In an aspect, the invention provides a set of rules for matching of clinical trials to biomarker status as determined by the molecular profiling described herein. In some embodiments, the matching of clinical trials to biomarker status is performed using one or more pre-specified criteria: 1) Trials are matched based on the OFF NCCN Compendia drug/drug class associated with potential benefit by the molecular profiling rules; 2) Trials are matched based on biomarker driven eligibility requirement of the trial; and 3) Trials are matched based on the molecular profile of the patient, the biology of the disease and the associated signaling pathways. In the latter case, i.e. item 3, clinical trial matching may comprise further criteria as follows. First, for directly targetable markers, match trials with agents directly targeting the gene (e.g., FGFR results map to anti-FGFR therapy trials; ERBB2 results map to anti-HER2 agents, etc). In addition, for directly targetable markers, trial matching considers downstream markers under the following scenarios: a) a known resistance mechanism is available (e.g., cMET inhibitors for EGFR gene); b) clinical evidence associates the (mutated) biomarker with drugs targeting downstream pathways (e.g., mTOR inhibitors when PIK3CA is mutated); and c) active clinical trials are enrolling patients (with the biomarker aberration in the inclusion criteria) with drugs targeting the downstream pathways (e.g., SMO inhibitors for BCR-ABL mutation T3151). In the case of markers that are not directly targetable by a known therapeutic agent, trial matching may consider alternative, downstream markers (e.g., platinum agents for ATM gene; MEK inhibitors for GNAS/GNAQ/GNA11 mutation). The clinical trials that are matched may be identified based on results of “pathogenic,” “presumed pathogenic,” or variant of uncertain (or unknown) significance (“VUS”). In some embodiments, the decision to incorporate/associate a drug class with a biomarker mutation can further depend on one or more of the following: 1) Clinical evidence; 2) Preclinical evidence; 3) Understanding of the biological pathway affected by the biomarker; and 4) expert analysis. In some embodiments, the mutation of biomarkers in the above section “Mutational Analysis” is linked to clinical trials using one or more of these criteria.

The guiding principle above can be used to identify classes of drugs that are linked to certain biomarkers. The biomarkers can be linked to various clinical trials that are studying these biomarkers, including without limitation requiring a certain biomarker status for clinical trial inclusion. Table 28 presents an illustrative overview of biomarker statuses that are matched to classes of drugs. In the table, the column headed “Biomarker” identifies that biomarker that is assessed according to the molecular profiling technique specified in the column headed “Technique.” It will be appreciated that equivalent methods can be used as desired. For example, Next Generation Sequencing (NGS; Next Gen SEQ) is used to identify mutations, but alternate nucleic acid sequencing and analysis techniques (Sanger sequencing, PCR, RFLP, etc) can be used in the alternative or in the conjunction. Results that indicate a potential match (e.g., a potential benefit) to a class of drugs are indicated in the column “Result.” For sequencing methods, “Pathogenic/Presumed Pathogenic/Variant of Unknown Significance” refer to mutations that are detected and are known, presumed, or potentially pathogenic. As appropriate, particular mutations or other alterations in the biomarker that are potentially matched to the class of drugs are identified in the column headed “Mutation Type/Alteration.” The matched drug classes are identified in the column headed “Drug Class (Associated Agents).” Associated agents are illustrative drugs that are members of the class. Clinical trials studying the drug classes and/or specific agents listed can be matched to the biomarker. In an aspect, the invention provides a method of selecting a clinical trial for enrollment of a patient, comprising performing molecular profiling of one or more biomarker on a sample from the patient using the methods described herein. For example, the profiling can be performed for one on more biomarker in Table 28 using the technique indicated in the table. The results of the profiling are matched to classes of drugs using the above criteria. Clinical trials studying members of the classes of drugs are identified. The matching between the biomarkers and the clinical trials can follow the rules in Table 29, which is described in more detail below. The patient is a potential candidate for the so-identified clinical trials.

TABLE 28
Biomarker - Drug Associations for Drugs in Matched Clinical Trials

| | | | | |
|---|---|---|---|---|
| | | | | Drug Class (Associated |
| | | | Mutation Type/ | Agents) matched by |
| Biomarker | Technique | Result | Alteration | clinical trials |

NGS tests

| | | | | |
|---|---|---|---|---|
| ATM | Next Gen | Pathogenic/Presumed | | PARP inhibitors (ABT-767, |
| | SEQ | Pathogenic/Variant | | CEP9722, E7016, iniparib, |
| | | of Unknown | | MK4827, olaparib, rucaparib, |
| | | Significance | | veliparib), |
| | | | | HDAC inhibitors |
| | | | | (abexinostat, ACY-1215, |
| | | | | AR-42, belinostat, CUDC- |
| | | | | 907, entinostat, FK228, |
| | | | | givinostat, JNJ26481585, |
| | | | | mocetinostat, panobinostat, |
| | | | | SHP-141, valproic acid, |
| | | | | vorinostat, 4SC-202) |
| | | | | Platinum compounds |
| | | | | (carboplatin, cisplatin, |
| | | | | oxaliplatin) |
| CSF1R | Next Gen | Pathogenic/Presumed | | FGFR TKI (dovitinib), |
| | SEQ | Pathogenic/Variant | | anti-CSFIR monoclonal |
| | | of Unknown | | antibody (IMC-CS4) |
| | | Significance | | |
| ERBB2 | Next Gen | Pathogenic/Presumed | | anti-HER2 monoclonal |
| | SEQ | Pathogenic/Variant | | antibody (pertuzumab, |
| | | of Unknown | | trastuzumab) |
| | | Significance | | HER2-targeted tyrosine |
| | | | | kinase inhibitors (afatinib, |
| | | | | dacomitinib, lapatinib, |
| | | | | neratinib) |
| | | | | anti-HER2 monoclonal |
| | | | | antibody - drug conjugate |
| | | | | (ado-trastuzumab emtansine |
| | | | | (T-DM1)) |
| GNAS | Next Gen | Pathogenic/Presumed | | MEK inhibitors (AZD8330, |
| | SEQ | Pathogenic/Variant | | BAY86-9766, CI-1040, |
| | | of Unknown | | GDC-0623, GDC-0973, |
| | | Significance | | MEK162, MSC1936369B, |
| | | | | MSC2015103B, PD0325901, |
| | | | | pimasertib (AS-703026), |
| | | | | selumetinib, TAK-733, |
| | | | | trametinib, XL518) |
| GNAQ | Next Gen | Pathogenic/Presumed | | MEK inhibitors (AZD8330, |
| | SEQ | Pathogenic/Variant | | BAY86-9766, CI-1040, |
| | | of Unknown | | GDC-0623, GDC-0973, |
| | | Significance | | MEK162, MSC1936369B, |
| | | | | MSC2015103B, PD0325901, |
| | | | | pimasertib (AS-703026), |
| | | | | selumetinib, TAK-733, |
| | | | | trametinib, XL518) |
| GNA11 | Next Gen | Pathogenic/Presumed | | MEK inhibitors (AZD8330, |
| | SEQ | Pathogenic/Variant | | BAY86-9766, CI-1040, |
| | | of Unknown | | GDC-0623, GDC-0973, |
| | | Significance | | MEK162, MSC1936369B, |
| | | | | MSC2015103B, PD0325901, |
| | | | | pimasertib (AS-703026), |
| | | | | selumetinib, TAK-733, |
| | | | | trametinib, XL518) |
| KDR | Next Gen | Pathogenic/Presumed | | VEGFR2-targeted tyrosine |
| | SEQ | Pathogenic/Variant | | kinase inhibitors (apatinib, |
| | | of Unknown | | axitinib, cabozantinib, |
| | | Significance | | famitinib, fruquintinib, |
| | | | | lenvatinib, motesanib, |
| | | | | ninedanib, pazopanib, |
| | | | | regorafenib, sorafenib, |
| | | | | sunitinib, tivozanib, |
| | | | | vandetanib, vatalanib) |
| | | | | anti-VEGFR2-targeted |
| | | | | monoclonal antibody |
| | | | | (ramucirumab, tanibirumab) |
| MLH1 | Next Gen | Pathogenic/Presumed | | PARP inhibitors (ABT-767, |
| | SEQ | Pathogenic/Variant | | CEP9722, E7016, iniparib, |
| | | of Unknown | | MK4827, olaparib, rucaparib, |
| | | Significance | | veliparib) |
| JAK3 | Next Gen | Pathogenic/Presumed | | no drugs |
| | SEQ | Pathogenic/Variant | | |
| | | of Unknown | | |
| | | Significance | | |
| PTPN11 | Next Gen | Pathogenic/Presumed | | no drugs |
| | SEQ | Pathogenic/Variant | | |
| | | of Unknown | | |
| | | Significance | | |
| RBI | Next Gen | Pathogenic/Presumed | | no drugs |
| | SEQ | Pathogenic/Variant | | |
| | | of Unknown | | |
| | | Significance | | |
| VHL | Next Gen | Pathogenic/Presumed | | VEGF, VEGFR targeted |
| | SEQ | Pathogenic/Variant | | therapies: Aflibercept, |
| | | of Unknown | | Axitinib, Bevacizumab, |
| | | Significance | | Cabozantinib, Pazopanib, |
| | | | | Regorafenib, Sorafenib, |
| | | | | Sunitinib, Tivozanib, |
| | | | | Apatinib, Famitinib, |
| | | | | Fruquintinib, Lenvatinib, |
| | | | | Motesanib, Ninedanib, |
| | | | | Vandetanib, Vatalanib, |
| | | | | Ramucirumab, Tanibirumab, |
| | | | | IMC-3C5, IMC-18F1 |
| | | | | PI3K/Akt/mTor |
| | | | | inhibitors: Temsirolimus, |
| | | | | Everolimus, CC-223, |
| | | | | Ridaforolimus, sirolimus, |
| | | | | MLN0128, GDC0941, |
| | | | | Deforolimus, BEZ235, DS- |
| | | | | 7423, GDC-0980, PF- |
| | | | | 04691502, PF-05212384, |
| | | | | SAR245409, BKM120, |
| | | | | BYL719, PX-866, GDC- |
| | | | | 0068, MK2206, |
| | | | | GSK2131795, GSK2110183, |
| | | | | GSK2141795, XL147 |
| | | | | (SAR245408), INK1117, |
| | | | | AZD5363, Perifosine, |
| | | | | ARQ092, AZD8055, OSI- |
| | | | | 027, BAY80-6946 |
| c-KIT | Next Gen | Pathogenic/Presumed | all mutations except | KIT inhibitiors: Sorafenib, |
| | SEQ | Pathogenic/Variant | V654A, T670I, D820A, | Dasatinib, Sunitinib, |
| | | of Unknown | D820E, D820G, | Nilotinib, Imatinib, |
| | | Significance | D820Y, N822H, | Regorafenib, Vatalanib, |
| | | | N822K, Y823D, | Masitinib, Pazopanib |
| | | | D816A, D816G, | |
| | | | D816H, D816V, A829P | |
| c-KIT | Next Gen | Pathogenic/Presumed | V654A, T670I, D820A, | KIT inhibitiors: Sorafenib, |
| | SEQ | Pathogenic/Variant | D820E, D820G, | Dasatinib, Sunitinib, |
| | | of Unknown | D820Y, N822H, | Nilotinib, Regorafenib, |
| | | Significance | N822K, Y823D, | Vatalanib, Masitinib, |
| | | | D816A, D816G, | Pazopanib |
| | | | D816H, D816V, A829P | |
| PDGFRA | Next Gen | Pathogenic/Presumed | all mutations except | PDGFRA inhibitors: |
| | SEQ | Pathogenic/Variant | D842V | Sorafenib, Dasatinib, |
| | | of Unknown | | Sunitinib, Nilotinib, |
| | | Significance | | Imatinib, Crenolanib (CP |
| | | | | 868-956), Masitinib, |
| | | | | Pazopanib |
| PDGFRA | Next Gen | Pathogenic/Presumed | D842V | PDGFRA inhibitors: |
| | SEQ | Pathogenic/Variant | | Sorafenib, Dasatinib, |
| | | of Unknown | | Sunitinib, Nilotinib, |
| | | Significance | | Crenolanib (CP 868-956), |
| | | | | Masitinib, Pazopanib |
| ABL1 | Next Gen | Pathogenic/Presumed | T315I | PI3K/Akt/mTor |
| | SEQ | Pathogenic/Variant | | inhibitors: Temsirolimus, |
| | | of Unknown | | Everolimus, CC-223, |
| | | Significance | | Ridaforolimus, sirolimus, |
| | | | | MLN0128, GDC0941, |
| | | | | Deforolimus, BEZ235, DS- |
| | | | | 7423, GDC-0980, PF- |
| | | | | 04691502, PF-05212384, |
| | | | | SAR245409, BKM120, |
| | | | | BYL719, PX-866, GDC- |
| | | | | 0068, MK2206, |
| | | | | GSK2131795, GSK2110183, |
| | | | | GSK2141795, |
| | | | | XL147(SAR245408), |
| | | | | INK1117, AZD5363, |
| | | | | Perifosine, ARQ092, |
| | | | | AZD8055, OSI-027, |
| | | | | BAY80-6946 |
| | | | | SMO antagonists: GDC- |
| | | | | 0449, LDE225, BMS833923 |
| ABL1 | Next Gen | Pathogenic/Presumed | all mutations except | PI3K/Akt/mTor |
| | SEQ | Pathogenic/Variant | T315I | inhibitors: Temsirolimus, |
| | | of Unknown | | Everolimus, CC-223, |
| | | Significance | | Ridaforolimus, sirolimus, |
| | | | | MLN0128, GDC0941, |
| | | | | Deforolimus, BEZ235, DS- |
| | | | | 7423, GDC-0980, PF- |
| | | | | 04691502, PF-05212384, |
| | | | | SAR245409, BKM120, |
| | | | | BYL719, PX-866, GDC- |
| | | | | 0068, MK2206, |
| | | | | GSK2131795, GSK2110183, |
| | | | | GSK2141795, XL147 |
| | | | | (SAR245408), INK1117, |
| | | | | AZD5363, Perifosine, |
| | | | | ARQ092, AZD8055, OSI- |
| | | | | 027, BAY80-6946 |
| | | | | SMO antagonists: GDC- |
| | | | | 0449, LDE225, BMS833923 |
| | | | | BCR-ABL inhibitors: |
| | | | | nilotinib, dasatinib, |
| | | | | ponatinib, bosutinib |
| cMET | Next Gen | Pathogenic/Presumed | | anti-HGF monoclonal |
| | SEQ | Pathogenic/Variant | | antibody (Ficlatuzumab, |
| | | of Unknown | | Rilotumumab, TAK-701) |
| | | Significance | | cMET-targeted inhibitors |
| | | | | (AMG-208, BMS-777607, |
| | | | | Compound 1 (Amgen), EMD |
| | | | | 1214063/EMD 1204831, |
| | | | | INC280, JNJ38877605, |
| | | | | Onartuzumab (MetMAb), |
| | | | | MK-2461, MK-8033, NK4, |
| | | | | PF4217903, PHA665752, |
| | | | | SGX126, Tivantinib (ARQ |
| | | | | 197), cabozantinib, |
| | | | | crizotinib, foretenib, |
| | | | | MGCD265) |
| FGFR1 | Next Gen | Pathogenic/Presumed | | Small molecule tyrosine |
| | SEQ | Pathogenic/Variant | | kinase inhibitors (TKI258, |
| | | of Unknown | | BIBF1120, BMS- |
| | | Significance | | 582,664(Brivanib), E7080, |
| | | | | TSU-68, AZD4547, |
| | | | | Dovitinib, E-3810, BGJ398, |
| | | | | TKI258, FP-1039, Ponatinib, |
| | | | | JNJ-42756493) |
| | | | | FGFR antibodies and FGF |
| | | | | ligand traps: (1A6, FP- |
| | | | | 1039) |
| FGFR2 | Next Gen | Pathogenic/Presumed | | Small molecule tyrosine |
| | SEQ | Pathogenic/Variant | | kinase inhibitors (TKI258, |
| | | of Unknown | | BIBF1120, BMS- |
| | | Significance | | 582,664(Brivanib), E7080, |
| | | | | TSU-68, AZD4547, |
| | | | | Dovitinib, E-3810, BGJ398, |
| | | | | TKI258, FP-1039, Ponatinib, |
| | | | | JNJ-42756493) |
| | | | | FGFR antibodies and FGF |
| | | | | ligand traps: (1A6, FP- |
| | | | | 1039) |
| RET | Next Gen | Pathogenic/Presumed | | RET inhibitors (Sorafenib, |
| | SEQ | Pathogenic/Variant | | sunitinib, motesanib, |
| | | of Unknown | | cabozantinib, vandetanib, |
| | | Significance | | lenvatinib) |
| CDH1 | Next Gen | Pathogenic/Presumed | | no drugs |
| | SEQ | Pathogenic/Variant | | |
| | | of Unknown | | |
| | | Significance | | |
| STK11 | Next Gen | Pathogenic/Presumed | | no drugs |
| | SEQ | Pathogenic/Variant | | |
| | | of Unknown | | |
| | | Significance | | |
| ERBB4 | Next Gen | Pathogenic/Presumed | | no drugs |
| | SEQ | Pathogenic/Variant | | |
| | | of Unknown | | |
| | | Significance | | |
| SMARCB1 | Next Gen | Pathogenic/Presumed | | no drugs |
| | SEQ | Pathogenic/Variant | | |
| | | of Unknown | | |
| | | Significance | | |
| PIK3CA | Next Gen | Pathogenic/Presumed | | PI3K/Akt/mTor |
| | SEQ | Pathogenic/Variant | | inhibitors: Temsirolimus, |
| | | of Unknown | | Everolimus, CC-223, |
| | | Significance | | Ridaforolimus, sirolimus, |
| | | | | MLN0128, GDC0941, |
| | | | | Deforolimus, BEZ235, DS- |
| | | | | 7423, GDC-0980, PF- |
| | | | | 04691502, PF-05212384, |
| | | | | SAR245409, BKM120, |
| | | | | BYL719, PX-866, GDC- |
| | | | | 0068, MK2206, |
| | | | | GSK2131795, GSK2110183, |
| | | | | GSK2141795, XL147 |
| | | | | (SAR245408), INK1117, |
| | | | | AZD5363, Perifosine, |
| | | | | ARQ092, AZD8055, OSI- |
| | | | | 027, BAY80-6946 |
| | | | | Aspirin: aspirin |
| PTEN | Next Gen | Pathogenic/Presumed | | PI3K/Akt/mTor |
| | SEQ | Pathogenic/Variant | | inhibitors: Temsirolimus, |
| | | of Unknown | | Everolimus, CC-223, |
| | | Significance | | Ridaforolimus, sirolimus, |
| | | | | MLN0128, GDC0941, |
| | | | | Deforolimus, BEZ235, DS- |
| | | | | 7423, GDC-0980, PF- |
| | | | | 04691502, PF-05212384, |
| | | | | SAR245409, BKM120, |
| | | | | BYL719, PX-866, GDC- |
| | | | | 0068, MK2206, |
| | | | | GSK2131795, GSK2110183, |
| | | | | GSK2141795, XL147 |
| | | | | (SAR245408), INK1117, |
| | | | | AZD5363, Perifosine, |
| | | | | ARQ092, AZD8055, OSI- |
| | | | | 027, BAY80-6946 |
| | | | | Parp inhibitors: ABT-767, |
| | | | | CEP9722, E7016, iniparib, |
| | | | | MK4827, olaparib, rucaparib, |
| | | | | veliparib, ABT-888 |
| AKT1 | Next Gen | Pathogenic/Presumed | | Akt inhibitors: AZD5363, |
| | SEQ | Pathogenic/Variant | | GDC-0068, MK2206, |
| | | of Unknown | | Perifosine, ARQ092 |
| | | Significance | | |
| ALK | Next Gen | Pathogenic/Presumed | | ALK inhibitors: crizotinib, |
| | SEQ | Pathogenic/Variant | | AP26113, X-396, |
| | | of Unknown | | CH5424802(AF-802), |
| | | Significance | | ASP3026, CEP-28122, CEP- |
| | | | | 37440, LDK378 |
| SMO | Next Gen | Pathogenic/Presumed | | SMO inhibitors: |
| | SEQ | Pathogenic/Variant | | Vismodegib, Erismodegib |
| | | of Unknown | | (LDE255), IPI-926, BMS- |
| | | Significance | | 838923, PF-04449913, |
| | | | | LEQ506, TAK441, |
| | | | | LY2940680. |
| KRAS | Next Gen | Pathogenic/Presumed | | MEK inhibitors: AZD8330, |
| | SEQ | Pathogenic/Variant | | BAY86-9766, CI-1040, |
| | | of Unknown | | GDC-0623, GDC-0973, |
| | | Significance | | MEK162, MSC1936369B, |
| | | | | MSC2015103B, PD0325901, |
| | | | | pimasertib (AS-703026), |
| | | | | selumetinib, TAK-733, |
| | | | | trametinib, XL518, ARRY- |
| | | | | 438162 |
| | | | | ERK inhibitors: |
| | | | | LY2228820, LY3007113, |
| | | | | BVD-523, BAY86-9766, |
| | | | | ARRY-614 |
| | | | | Regorafenib: regorafenib |
| NRAS | Next Gen | Pathogenic/Presumed | | MEK inhibitors: AZD8330, |
| | SEQ | Pathogenic/Variant | | BAY86-9766, CI-1040, |
| | | of Unknown | | GDC-0623, GDC-0973, |
| | | Significance | | MEK162, MSC1936369B, |
| | | | | MSC2015103B, PD0325901, |
| | | | | pimasertib (AS-703026), |
| | | | | selumetinib, TAK-733, |
| | | | | trametinib, XL518, ARRY- |
| | | | | 438162 |
| | | | | ERK inhibitors: |
| | | | | LY2228820, LY3007113, |
| | | | | BVD-523, BAY86-9766, |
| | | | | ARRY-614 |
| HRAS | Next Gen | Pathogenic/Presumed | | MEK inhibitors: AZD8330, |
| | SEQ | Pathogenic/Variant | | BAY86-9766, CI-1040, |
| | | of Unknown | | GDC-0623, GDC-0973, |
| | | Significance | | MEK162, MSC1936369B, |
| | | | | MSC2015103B, PD0325901, |
| | | | | pimasertib (AS-703026), |
| | | | | selumetinib, TAK-733, |
| | | | | trametinib, XL518, ARRY- |
| | | | | 438162 |
| | | | | ERK inhibitors: |
| | | | | LY2228820, LY3007113, |
| | | | | BVD-523, BAY86-9766, |
| | | | | ARRY-614 |
| SMAD4 | Next Gen | Pathogenic/Presumed | | no drugs |
| | SEQ | Pathogenic/Variant | | |
| | | of Unknown | | |
| | | Significance | | |
| IDH1 | Next Gen | Pathogenic/Presumed | | Alkylating agents: |
| | SEQ | Pathogenic/Variant | | temozolomide, dacarbazine |
| | | of Unknown | | Hypomethylating agents: |
| | | Significance | | azacitidine, decitabine |
| JAK2 | Next Gen | Pathogenic/Presumed | | JAK2 inhibitors: |
| | SEQ | Pathogenic/Variant | | ruxolitinib, tg101348 |
| | | of Unknown | | (panolosetron), CEP-701 |
| | | Significance | | (lestaurtinib), NS-018, |
| | | | | LY278544 |
| MPL | Next Gen | Pathogenic/Presumed | | JAK2 inhibitors: |
| | SEQ | Pathogenic/Variant | | ruxolitinib, tg101348 |
| | | of Unknown | | (panolosetron), CEP-701 |
| | | Significance | | (lestaurtinib), NS-018, |
| | | | | LY278544 |
| FLT3 | Next Gen | Pathogenic/Presumed | | FLT3 inhibitors: CEP-701 |
| | SEQ | Pathogenic/Variant | | (lestaurtinib), sunitinib, |
| | | of Unknown | | MLN518 (tandutinib), |
| | | Significance | | PKC412 (midostaurin) |
| NPM1 | Next Gen | Pathogenic/Presumed | | no drugs |
| | SEQ | Pathogenic/Variant | | |
| | | of Unknown | | |
| | | Significance | | |
| APC | Next Gen | Pathogenic/Presumed | | Wnt pathway inhibitors: |
| | SEQ | Pathogenic/Variant | | PRI-724 |
| | | of Unknown | | |
| | | Significance | | |
| CTNNB1 | Next Gen | Pathogenic/Presumed | | Wnt pathway inhibitors: |
| | SEQ | Pathogenic/Variant | | PRI-724 |
| | | of Unknown | | |
| | | Significance | | |
| FBXW7 | Next Gen | Pathogenic/Presumed | | no drugs |
| | SEQ | Pathogenic/Variant | | |
| | | of Unknown | | |
| | | Significance | | |
| BRAF | Next Gen | Pathogenic/Presumed | | BRAF inhibitors: sorafenib, |
| | SEQ | Pathogenic/Variant | | vemurafenib, RAF-265, |
| | | of Unknown | | XL281, LGX818, |
| | | Significance | | GSK2118436 (dabrafenib), |
| | | | | ARQ736, RO5212054 |
| | | | | MEK inhibitors: AZD8330, |
| | | | | BAY86-9766, CI-1040, |
| | | | | GDC-0623, GDC-0973, |
| | | | | MEK162, MSC1936369B, |
| | | | | MSC2015103B, PD0325901, |
| | | | | pimasertib (AS-703026), |
| | | | | selumetinib, TAK-733, |
| | | | | trametinib, XL518, ARRY- |
| | | | | 438162 |
| | | | | ERK inhibitors: |
| | | | | LY2228820, LY3007113, |
| | | | | BVD-523, BAY86-9766, |
| | | | | ARRY-614 |
| HNF1A | Next Gen | Pathogenic/Presumed | | no drugs |
| | SEQ | Pathogenic/Variant | | |
| | | of Unknown | | |
| | | Significance | | |
| EGFR | Next Gen | Pathogenic/Presumed | T790M; exon20 insert | Pan HER inhibitors: |
| | SEQ | Pathogenic/Variant | (A763_Y764insFQEA, | (afatinib, dacomitinib, CO- |
| | | of Unknown | A767_D770dup, | 1686, XL647, neratinib, |
| | | Significance | A767_V769dup, | BMS-690514, Icotinib, |
| | | | D770delinsGY, | poziotinib) |
| | | | D770dup, | |
| | | | D770_N771insG, | |
| | | | D770_N771insGF, | |
| | | | D770_N771insGT, | |
| | | | D770_N771insGY, | |
| | | | D770_N771insNPH, | |
| | | | D770_P772delinsKG, | |
| | | | H770dup, H773dup, | |
| | | | H773_V774dup, | |
| | | | H773_ V774insAH, | |
| | | | H773_V774insY, | |
| | | | N771delinsGF, | |
| | | | N771delinsGY, | |
| | | | N771delinsKG, | |
| | | | N771delinsRY, | |
| | | | N771_ H773delinsTGG, | |
| | | | N771_H773dup, | |
| | | | N771_P772insH, | |
| | | | P772_H773insGNP, | |
| | | | S768_D770dup, | |
| | | | V769_ D770dup, | |
| | | | V769_ D770insDNP, | |
| | | | V769_ D770insGG, | |
| | | | V769_ D770insVTW, | |
| | | | Y764_V765insHH) | |
| EGFR | Next Gen | Pathogenic/Presumed | all mutations except | Pan HER inhibitors: |
| | SEQ | Pathogenic/Variant | T790M and exon20 | (afatinib, dacomitinib, CO- |
| | | of Unknown | insert | 1686, XL647, neratinib, |
| | | Significance | (A763_ Y764insFQEA, | BMS-690514, Icotinib, |
| | | | A767_ D770dup, | poziotinib) |
| | | | A767_ V769dup, | EGFR TKIs: (erlotinib, |
| | | | D770delinsGY, | gefitinib) |
| | | | D770dup, | |
| | | | D770_ N771insG, | |
| | | | D770_ N771insGF, | |
| | | | D770_ N771insGT, | |
| | | | D770_ N771insGY, | |
| | | | D770_ N771insNPH, | |
| | | | D770_ P772delinsKG, | |
| | | | H770dup, H773dup, | |
| | | | H773_V774dup, | |
| | | | H773_V774insAH, | |
| | | | H773_ V774insY, | |
| | | | N771delinsGF, | |
| | | | N771delinsGY, | |
| | | | N771delinsKG, | |
| | | | N771delinsRY, | |
| | | | N771_ H773delinsTGG, | |
| | | | N771_ H773dup, | |
| | | | N771_ P772insH, | |
| | | | P772_H773insGNP, | |
| | | | S768_D770dup, | |
| | | | V769_ D770dup, | |
| | | | V769_D770insDNP, | |
| | | | V769_D770insGG, | |
| | | | V769_D770insVTW, | |
| | | | Y764_V765insHH) | |
| EGFR | Next Gen | Present | | Pan HER inhibitors: |
| T790M | SEQ | | | (afatinib, dacomitinib, CO- |
| | | | | 1686, XL647, neratinib, |
| | | | | BMS-690514, Icotinib, |
| | | | | poziotinib) |
| NOTCH1 | Next Gen | Pathogenic/Presumed | | HDAC inhibitors: HDAC |
| | SEQ | Pathogenic/Variant | | inhibitors (abexinostat, |
| | | of Unknown | | ACY-1215, AR-42, |
| | | Significance | | belinostat, CUDC-907, |
| | | | | entinostat, FK228, givinostat, |
| | | | | JNJ26481585, mocetinostat, |
| | | | | panobinostat, SHP-141, |
| | | | | valproic acid, vorinostat, |
| | | | | 4SC-202) |
| | | | | GSI: (MK0752, RO4929097, |
| | | | | R4733, BMS-906024, PF- |
| | | | | 03084014, MEDI0639) |
| TP53 | Next Gen | Pathogenic/Presumed | | WEE1 inhibitors: MK-1775 |
| | SEQ | Pathogenic/Variant | | CHK1 inhibitors: |
| | | of Unknown | | LY2606368, SCH 900776 |
| | | Significance | | Biologicals (gene therapy, |
| | | | | vaccines): rAd-p53, P53- |
| | | | | SLP, Ad5CMV-p53, |
| | | | | adenovirus-p53 transduced |
| | | | | dendritic cell vaccine |
| | | | | (Ad.p53-DC vaccines), |
| | | | | modified vaccinia virus |
| | | | | ankara vaccine expressing |
| | | | | p53, ALT-801 |
| | | | | p53 activators: PRIMA |
| TP53 | Next Gen | Wild Type | | P53-MDM2 interaction |
| | SEQ | | | inhibitors: CGM097, |
| | | | | RO5503781, RO5045337, |
| | | | | Kevetrin |
| | | | | (thioureidobutyronitrile), DS- |
| | | | | 3032 |

Sanger SEQ

| | | | |
|---|---|---|---|
| PIK3CA | Sanger | Exon 20; Exon 9; | PI3K/Akt/mTor |
| | SEQ | Mutated - Other | inhibitors: Temsirolimus, |
| | | | Everolimus, CC-223, |
| | | | Ridaforolimus, sirolimus, |
| | | | MLN0128, GDC0941, |
| | | | Deforolimus, BEZ235, DS- |
| | | | 7423, GDC-0980, PF- |
| | | | 04691502, PF-05212384, |
| | | | SAR245409, BKM120, |
| | | | BYL719, PX-866, GDC- |
| | | | 0068, MK2206, |
| | | | GSK2131795, GSK2110183, |
| | | | GSK2141795, XL147 |
| | | | (SAR245408), INK1117, |
| | | | AZD5363, Perifosine, |
| | | | ARQ092, AZD8055, OSI- |
| | | | 027, BAY80-6946 |
| | | | Aspirin: aspirin |
| KRAS | Sanger | G12, G13; G13D; | MEK inhibitors: AZD8330, |
| | SEQ | Q61; Mutated - | BAY86-9766, CI-1040, |
| | | Other | GDC-0623, GDC-0973, |
| | | | MEK162, MSC1936369B, |
| | | | MSC2015103B, PD0325901, |
| | | | pimasertib (AS-703026), |
| | | | selumetinib, TAK-733, |
| | | | trametinib, XL518, ARRY- |
| | | | 438162 |
| | | | ERK inhibitors: |
| | | | LY2228820, LY3007113, |
| | | | BVD-523, BAY86-9766, |
| | | | ARRY-614 |
| | | | Regorafenib: regorafenib |
| KRAS | Sanger | Present | MEK inhibitors: AZD8330, |
| G13D | SEQ | | BAY86-9766, CI-1040, |
| | | | GDC-0623, GDC-0973, |
| | | | MEK162, MSC1936369B, |
| | | | MSC2015103B, PD0325901, |
| | | | pimasertib (AS-703026), |
| | | | selumetinib, TAK-733, |
| | | | trametinib, XL518, ARRY- |
| | | | 438162 |
| | | | ERK inhibitors: |
| | | | LY2228820, LY3007113, |
| | | | BVD-523, BAY86-9766, |
| | | | ARRY-614 |
| | | | Regorafenib: regorafenib |
| NRAS | Sanger | G12, G13; Q61; | MEK inhibitors: AZD8330, |
| | SEQ | Mutated - Other | BAY86-9766, CI-1040, |
| | | | GDC-0623, GDC-0973, |
| | | | MEK162, MSC1936369B, |
| | | | MSC2015103B, PD0325901, |
| | | | pimasertib (AS-703026), |
| | | | selumetinib, TAK-733, |
| | | | trametinib, XL518, ARRY- |
| | | | 438162 |
| | | | ERK inhibitors: |
| | | | LY2228820, LY3007113, |
| | | | BVD-523, BAY86-9766, |
| | | | ARRY-614 |
| BRAF | Sanger | V600D; V600E; | BRAF inhibitors: sorafenib, |
| | SEQ | V600K; V600R; | vemurafenib, RAF-265, |
| | | Exon11; Mutated - | XL281, LGX818, |
| | | Other; SEQ- | GSK2118436 (dabrafenib), |
| | | MUT/PCR-WT; | ARQ736, RO5212054 |
| | | SEQ-WT/PCR- | MEK inhibitors: AZD8330, |
| | | MUT; | BAY86-9766, CI-1040, |
| | | | GDC-0623, GDC-0973, |
| | | | MEK162, MSC1936369B, |
| | | | MSC2015103B, PD0325901, |
| | | | pimasertib (AS-703026), |
| | | | selumetinib, TAK-733, |
| | | | trametinib, XL518, ARRY- |
| | | | 438162 |
| | | | ERK inhibitors: |
| | | | LY2228820, LY3007113, |
| | | | BVD-523, BAY86-9766, |
| | | | ARRY-614 |
| EGFR | Sanger | Exon 18 G719A; | EGFR TKIs: (erlotinib, |
| | SEQ and | Exon 19 del; Exon | gefitinib) |
| | RFLP | 20R776; Exon 21 | Pan HER inhibitors: |
| | | L858R; Exon 21 | (afatinib, dacomitinib, CO- |
| | | L861; | 1686, XL647, neratinib, |
| | | | BMS-690514, Icotinib, |
| | | | poziotinib) |
| EGFR | Sanger | Present | Pan HER inhibitors: |
| T790M | SEQ and | | (afatinib, dacomitinib, CO- |
| | RFLP | | 1686, XL647, neratinib, |
| | | | BMS-690514, Icotinib, |
| | | | poziotinib) |
| EGFR | Sanger | Present | Pan HER inhibitors: |
| Exon 20 | SEQ and | | (afatinib, dacomitinib, CO- |
| ins | RFLP | | 1686, XL647, neratinib, |
| | | | BMS-690514, Icotinib, |
| | | | poziotinib) |
| IDH2 | Sanger | Mutated-Other, | Alkylating agents: |
| | SEQ | R140, R172 | temozolomide, dacarbazine |
| | | | Hypomethylating agents: |
| | | | azacitidine, decitabine |

IHC Tests

| | | | |
|---|---|---|---|
| Her2/Neu | IHC | Positive | anti-HER2 monoclonal |
| | | | antibody (pertuzumab, |
| | | | trastuzumab) |
| | | | HER2-targeted tyrosine |
| | | | kinase inhibitors (afatinib, |
| | | | dacomitinib, lapatinib, |
| | | | neratinib) |
| | | | anti-HER2 monoclonal |
| | | | antibody - drug conjugate |
| | | | (ado-trastuzumab emtansine |
| | | | (T-DM1)) |
| cMET | IHC | Positive | anti-HGF monoclonal |
| | | | antibody (Ficlatuzumab, |
| | | | Rilotumumab, TAK-701) |
| | | | cMET-targeted inhibitors |
| | | | (AMG-208, BMS-777607, |
| | | | Compound 1 (Amgen), EMD |
| | | | 1214063/EMD 1204831, |
| | | | INC280, JNJ38877605, |
| | | | Onartuzumab (MetMAb), |
| | | | MK-2461, MK-8033, NK4, |
| | | | PF4217903, PHA665752, |
| | | | SGX126, Tivantinib (ARQ |
| | | | 197), cabozantinib, |
| | | | crizotinib, foretenib, |
| | | | MGCD265) |
| | | | cMET antibody: ABT-700 |
| PTEN | IHC | Negative | PI3K/Akt/mTor |
| | | | inhibitors: Temsirolimus, |
| | | | Everolimus, CC-223, |
| | | | Ridaforolimus, sirolimus, |
| | | | MLN0128, GDC0941, |
| | | | Deforolimus, BEZ235, DS- |
| | | | 7423, GDC-0980, PF- |
| | | | 04691502, PF-05212384, |
| | | | SAR245409, BKM120, |
| | | | BYL719, PX-866, GDC- |
| | | | 0068, MK2206, |
| | | | GSK2131795, GSK2110183, |
| | | | GSK2141795, XL147 |
| | | | (SAR245408), INK1117, |
| | | | AZD5363, Perifosine, |
| | | | ARQ092, AZD8055, OSI- |
| | | | 027, BAY80-6946 |
| | | | Parp inhibitors: ABT-767, |
| | | | CEP9722, E7016, iniparib, |
| | | | MK4827, olaparib, rucaparib, |
| | | | veliparib, ABT-888 |
| Androgen | IHC | positive | Anti androgens: |
| Receptor | | | (Bicalutamide, flutamide, |
| | | | abiraterone, enzalutamide, |
| | | | TAK-700, ARN-509) GnRH |
| | | | agonists/antagonists: |
| | | | (goserelin, leuprolide, |
| | | | degarelix, abarelix); |
| EGFR | IHC | Positive | EGFR monoclonal |
| | | | antibody: cetuximab, |
| | | | nimotuzumab |

CISH/FISH Tests

| | | | |
|---|---|---|---|
| Her2/Neu | CISH/FISH | Amplified | anti-HER2 monoclonal |
| | | | antibody (pertuzumab, |
| | | | trastuzumab) |
| | | | HER2-targeted tyrosine |
| | | | kinase inhibitors (afatinib, |
| | | | dacomitinib, lapatinib, |
| | | | neratinib) |
| | | | anti-HER2 monoclonal |
| | | | antibody - drug conjugate |
| | | | (ado-trastuzumab emtansine |
| | | | (T-DM1)) |
| cMET | CISH/FISH | Amplified | anti-HGF monoclonal |
| | | | antibody (Ficlatuzumab, |
| | | | Rilotumumab, TAK-701) |
| | | | cMET-targeted inhibitors |
| | | | (AMG-208, BMS-777607, |
| | | | Compound 1 (Amgen), EMD |
| | | | 1214063/EMD 1204831, |
| | | | INC280, JNJ38877605, |
| | | | Onartuzumab (MetMAb), |
| | | | MK-2461, MK-8033, NK4, |
| | | | PF4217903, PHA665752, |
| | | | SGX126, Tivantinib (ARQ |
| | | | 197), cabozantinib, |
| | | | crizotinib, foretenib, |
| | | | MGCD265) |
| | | | cMET antibody: ABT-700 |
| ALK | FISH | Positive | ALK inhibitors: crizotinib, |
| | | | AP26113, X-396, |
| | | | CH5424802(AF-802), |
| | | | ASP3026, CEP-28122, CEP- |
| | | | 37440, LDK378 |
| | | | HSP90 inhibitors: AUY922, |
| | | | Ganetespib, 17-AGG |

Cobas PCR

| | | | |
|---|---|---|---|
| BRAF | Cobas PCR | V600E | BRAF inhibitors: sorafenib, |
| | (qPCR) | | vemurafenib, RAF-265, |
| | | | XL281, LGX818, |
| | | | GSK2118436 (dabrafenib), |
| | | | ARQ736, RO5212054 |
| | | | MEK inhibitors: AZD8330, |
| | | | BAY86-9766, CI-1040, |
| | | | GDC-0623, GDC-0973, |
| | | | MEK162, MSC1936369B, |
| | | | MSC2015103B, PD0325901, |
| | | | pimasertib (AS-703026), |
| | | | selumetinib, TAK-733, |
| | | | trametinib, XL518, ARRY- |
| | | | 438162 |
| | | | ERK inhibitors: |
| | | | LY2228820, LY3007113, |
| | | | BVD-523, BAY86-9766, |
| | | | ARRY-614 |

Fragment Analysis

| | | | |
|---|---|---|---|
| EGFRvIII | Fragment | present | EGFRvIII targeted peptide |
| | Analysis | | vaccine: rindopepimut |
| | | | (CDX-110; PEP-3-KLH) |
| | | | EGFRvIII targeted |
| | | | antibodies and antibody |
| | | | conjugates: ABT-806 |
| | | | (mAb806), ABT-414, AMG |
| | | | 595 |
| | | | EGFR TKIs: erlotinib, |
| | | | gefitinib |
| | | | Pan HER inhibitors: |
| | | | afatinib, dacomitinib, CO- |
| | | | 1686, XL647, neratinib, |
| | | | BMS-690514, Icotinib, |
| | | | poziotinib |

As noted herein, the status of various biomarkers assessed by molecular profiling of the invention can be used to match a patient with a given biomarker status to a clinical trial. Table 29 provides illustrative rules that can be followed to match various clinical trials. In the table, the column headed “NCT Number” provides a unique identifier for each trial ongoing in the United States at clinicaltrials.gov. Every study in ClinicalTrials.gov is assigned a unique number called the ClinicalTrials.gov Identifier (NCT Number). For example, a trial can be accessed on the internet by following the URL convention “clinicaltrials.gov/show/[NCT Number]” where “[NCT Number]” is replaced by the NCT Number of the trial, such as indicated in the table. The column “Tumor Type” indicates what lineages or lineages of tumor are being studied in the clinical trial. The column “Drug” indicates what drug or drugs are being studied in the clinical trial. The column “Biomarker” indicates the biomarkers that are considered by the trial. The various columns “Test” and “Result” indicate the test results for the one or more biomarkers whose status is used to determine eligibility for the clinical trial. If the test results are matched in a given row in Table 29, then the patient is indicated as a candidate for a clinical trial. By way of non-limiting example, consider the first row in Table 29 underneath the headers. A colorectal adenocarcinoma is assessed according to the systems and methods of the invention. A V600E or V600K mutation in BRAF is identified by sequencing. The patient may be eligible for inclusion in the clinical trial identified by the NCT Number NCT00326495, which trial is studying the drug sorafenib. The same patient may also be eligible for the clinical trial identified by the NCT Number NCT00625378, which trial is studying the drug sorafenib in all cancer lineages having a V600E or V600K mutation in BRAF. Similar logic is followed when multiple biomarkers are implicated in the rule set in Table 29. In such cases, all of the biomarkers and test results thereof must be matched in order to suggest the patient as eligible for enrollment in the clinical trial.

Abbreviations in Table 29 are as found elsewhere herein, e.g.: Seq. (sequencing); BAC (bronchioloalveolar cancer); NSCLC (non-small cell lung cancer); IHC (immunohistochemistry); ISH (in situ hybridization).

The rule set in Table 29 can be updated, as new trials linking various biomarkers to enrollment eligibility become available. If the biomarkers are not already assessed as part of the molecular profiles provided by the invention, then the biomarkers can be added to the molecular profiles.

TABLE 29
Rules for Clinical Trial Selection based on Biomarker Assessment

| NCT Number | Tumor Type | Drug | Biomarker | Test | Result | Test |
|---|---|---|---|---|---|---|
| NCT00326495 | Colorectal | sorafenib | BRAF | Seq. BRAF | Mutated \| | |
| | Adenocarcinoma | | | | V600E \| | |
| | | | | | V600K | |
| NCT00326495 | Colorectal | sorafenib | BRAF | PCR BRAF | V600E | |
| | Adenocarcinoma | | | | | |
| NCT00574769 | Prostatic | everolimus | PIK3CA, | Seq. | Mutated \| | |
| | Adenocarcinoma | | PTEN | PIK3CA | exon20 | |
| NCT00574769 | Prostatic | everolimus | PIK3CA, | Seq. PTEN | Mutated | |
| | Adenocarcinoma | | PTEN | | | |
| NCT00574769 | Prostatic | bevacizumab | VHL | Seq. VHL | Mutated | |
| | adenocarcinoma | | | | | |
| NCT00585195 | All | crizotinib | ALK | ISH ALK | Positive | |
| NCT00585195 | All | erizotinib | ROS1 | ISH ROS1 | Positive | |
| NCT00610948 | All | everolimus | PIK3CA, | Seq. | Mutated \| | |
| | | | PTEN | PIK3CA | exon20 | |
| NCT00610948 | All | everolimus | PIK3CA, | Seq. PTEN | Mutated | |
| | | | PTEN | | | |
| NCT00622466 | Breast Carcinoma | sorafenib | VHL | Seq. VHL | Mutated | |
| NCT00625378 | All | sorafenib | BRAF | Seq. BRAF | Mutated \| | |
| | | | | | V600E \| | |
| | | | | | V600K | |
| NCT00625378 | All | sorafenib | BRAF | PCR BRAF | V600E | |
| NCT00756340 | All | everolimus | PIK3CA, | Seq. | Mutated \| | |
| | | | PTEN | PIK3CA | exon20 | |
| NCT00756340 | All | everolimus | PIK3CA, | Seq. PTEN | Mutated | |
| | | | PTEN | | | |
| NCT00770263 | All | temsirolimus | PIK3CA, | Seq. | Mutated \| | |
| | | | PTEN | PIK3CA | exon20 | |
| NCT00770263 | All | temsirolimus | PIK3CA, | Seq. PTEN | Mutated | |
| | | | PTEN | | | |
| NCT00780494 | Gastric | bevacizumab | VHL | Seq. VHL | Mutated | |
| | adenocarcinoma, | | | | | |
| | Esophageal and | | | | | |
| | Esophagogastric | | | | | |
| | Junction | | | | | |
| | Carcinoma | | | | | |
| NCT00912340 | Breast Carcinoma | everolimus | ER, PR, | IHC ER | Positive | IHC Her2 |
| | | | Her2 | | | |
| NCT00912340 | Breast Carcinoma | everolimus | ER, PR, | IHC ER | Positive | IHC Her2 |
| | | | Her2 | | | |
| NCT00912340 | Breast Carcinoma | everolimus | ER, PR, | IHC ER | Positive | IHC Her2 |
| | | | Her2 | | | |
| NCT00912340 | Breast Carcinoma | everolimus | ER, PR, | IHC ER | Positive | IHC Her2 |
| | | | Her2 | | | |
| NCT00912340 | Breast Carcinoma | everolimus | ER, PR, | IHC ER | Positive | IHC Her2 |
| | | | Her2 | | | |
| NCT00912340 | Breast Carcinoma | everolimus | ER, PR, | IHC ER | Positive | IHC Her2 |
| | | | Her2 | | | |
| NCT00912340 | Breast Carcinoma | everolimus | ER, PR, | IHC ER | Positive | IHC Her2 |
| | | | Her2 | | | |
| NCT00912340 | Breast Carcinoma | everolimus | ER, PR, | IHC ER | Positive | IHC Her2 |
| | | | Her2 | | | |
| NCT00912340 | Breast Carcinoma | everolimus | ER, PR, | IHC ER | Positive | IHC Her2 |
| | | | Her2 | | | |
| NCT00912340 | Breast Carcinoma | everolimus | ER, PR, | IHC PR | Positive | IHC Her2 |
| | | | Her2 | | | |
| NCT00912340 | Breast Carcinoma | everolimus | ER, PR, | IHC PR | Positive | IHC Her2 |
| | | | Her2 | | | |
| NCT00912340 | Breast Carcinoma | everolimus | ER, PR, | IHC PR | Positive | IHC Her2 |
| | | | Her2 | | | |
| NCT00912340 | Breast Carcinoma | everolimus | ER, PR, | IHC PR | Positive | IHC Her2 |
| | | | Her2 | | | |
| NCT00912340 | Breast Carcinoma | everolimus | ER, PR, | IHC PR | Positive | IHC Her2 |
| | | | Her2 | | | |
| NCT00912340 | Breast Carcinoma | everolimus | ER, PR, | IHC PR | Positive | IHC Her2 |
| | | | Her2 | | | |
| NCT00912340 | Breast Carcinoma | everolimus | ER, PR, | IHC PR | Positive | IHC Her2 |
| | | | Her2 | | | |
| NCT00912340 | Breast Carcinoma | everolimus | ER, PR, | IHC PR | Positive | IHC Her2 |
| | | | Her2 | | | |
| NCT00912340 | Breast Carcinoma | everolimus | ER, PR, | IHC PR | Positive | IHC Her2 |
| | | | Her2 | | | |
| NCT00934895 | Breast Carcinoma | everolimus | Her2 | IHC Her2 | No Data | ISH Her2 |
| NCT00934895 | Breast Carcinoma | everolimus | Her2 | IHC Her2 | No Data | ISH Her2 |
| NCT00934895 | Breast Carcinoma | everolimus | Her2 | IHC Her2 | Equiv. | ISH Her2 |
| NCT00934895 | Breast Carcinoma | everolimus | Her2 | IHC Her2 | Equiv. | ISH Her2 |
| NCT00934895 | Breast Carcinoma | everolimus | Her2 | IHC Her2 | Negative | ISH Her2 |
| NCT00934895 | Breast Carcinoma | everolimus | Her2 | IHC Her2 | Negative | ISH Her2 |
| NCT00934895 | Breast Carcinoma | everolimus | Her2 | IHC Her2 | Negative | ISH Her2 |
| NCT00936858 | Thyroid | everolimus | PIK3CA, | Seq. | Mutated \| | |
| | Carcinoma | | PTEN | PIK3CA | exon20 | |
| NCT00936858 | Thyroid | everolimus | PIK3CA, | Seq. PTEN | Mutated | |
| | Carcinoma | | PTEN | | | |
| NCT00976573 | Melanoma | everolimus | PIK3CA, | Seq. | Mutated \| | |
| | | | PTEN | PIK3CA | exon20 | |
| NCT00976573 | Melanoma | everolimus | PIK3CA, | Seq. PTEN | Mutated | |
| | | | PTEN | | | |
| NCT01006369 | Colorectal | bevacizumab | VHL | Seq. VHL | Mutated | |
| | Adenocarcinoma | | | | | |
| NCT01014936 | All | EMD 1214063 | cMET | ISH CMET | Amplified | |
| NCT01014936 | All | EMD 1214063 | cMET | IHC CMET | Positive | |
| NCT01031381 | Ovarian Surface | everolimus | PIK3CA, | Seq. | Mutated \| | |
| | Epithelial | | PTEN | PIK3CA | exon20 | |
| | Carcinomas | | | | | |
| NCT01031381 | Ovarian Surface | everolimus | PIK3CA, | Seq. PTEN | Mutated | |
| | Epithelial | | PTEN | | | |
| | Carcinomas | | | | | |
| NCT01047293 | Colorectal | bevacizumab | VHL | Seq. VHL | Mutated | |
| | Adenocarcinoma | | | | | |
| NCT01061788 | All | everolimus | PIK3CA, | Seq. | Mutated \| | |
| | | | PTEN | PIK3CA | exon20 | |
| NCT01061788 | All | everolimus | PIK3CA, | Seq. PTEN | Mutated | |
| | | | PTEN | | | |
| NCT01087554 | All | sirolimus | PIK3CA, | Seq. | Mutated \| | |
| | | | PTEN | PIK3CA | exon20 | |
| NCT01087554 | All | sirolimus | PIK3CA, | Seq. PTEN | Mutated | |
| | | | PTEN | | | |
| NCT01087983 | All | sirolimus, | PIK3CA, | Seq. | Mutated \| | |
| | | metformin | PTEN | PIK3CA | exon20 | |
| NCT01087983 | All | sirolimus, | PIK3CA, | Seq. PTEN | Mutated | |
| | | metformin | PTEN | | | |
| NCT01089101 | Glioblastoma | AZD6244 | KRAS, | Seq. KRAS | Mutated \| | |
| | | | NRAS, | | G13D | |
| | | | BRAF | | | |
| NCT01089101 | Glioblastoma | AZD6244 | KRAS, | Seq. BRAF | Mutated \| | |
| | | | NRAS, | | V600E \| | |
| | | | BRAF | | V600K | |
| NCT01089101 | Glioblastoma | AZD6244 | KRAS, | Seq. NRAS | Mutated | |
| | | | NRAS, | | | |
| | | | BRAF | | | |
| NCT01111825 | Breast Carcinoma | temsirolimus | Her2 | IHC Her2 | Positive | |
| NCT01111825 | Breast Carcinoma | temsirolimus | Her2 | ISH Her2 | Amplified | |
| NCT01111825 | Breast Carcinoma | temsirolimus | Her2 | ISH Her2 | Equiv. | |
| | | | | | High | |
| NCT01111825 | Breast Carcinoma | temsirolimus | ER, PR, | IHC ER | Negative | IHC PR |
| | | | Her2 | | | |
| NCT01111825 | Breast Carcinoma | temsirolimus | ER, PR, | IHC ER | Negative | IHC PR |
| | | | Her2 | | | |
| NCT01111825 | Breast Carcinoma | temsirolimus | ER, PR, | IHC ER | Negative | IHC PR |
| | | | Her2 | | | |
| NCT01111825 | Breast Carcinoma | temsirolimus | ER, PR, | IHC ER | Negative | IHC PR |
| | | | Her2 | | | |
| NCT01111825 | Breast Carcinoma | temsirolimus | ER, PR, | IHC ER | Negative | IHC PR |
| | | | Her2 | | | |
| NCT01111825 | Breast Carcinoma | temsirolimus | ER, PR, | IHC ER | Negative | IHC PR |
| | | | Her2 | | | |
| NCT01111825 | Breast Carcinoma | temsirolimus | ER, PR, | IHC ER | Negative | IHC PR |
| | | | Her2 | | | |
| NCT01121575 | BAC, NSCLC | crizotinib | cMET | ISH CMET | Amplified | |
| NCT01121575 | BAC, NSCLC | crizotinib | cMET | IHC CMET | Positive | |
| NCT01122199 | All | everolimus | PIK3CA, | Seq. | Mutated \| | |
| | | | PTEN | PIK3CA | exon20 | |
| NCT01122199 | All | everolimus | PIK3CA, | Seq. PTEN | Mutated | |
| | | | PTEN | | | |
| NCT01132664 | Breast carcinoma | BKM120 | PIK3CA, | Seq. | Mutated \| | IHC |
| | | | PTEN | PIK3CA | exon20 | HER2/Neu |
| NCT01132664 | Breast carcinoma | BKM120 | PIK3CA, | Seq. | Mutated \| | ISH |
| | | | PTEN | PIK3CA | exon20 | HER2/Neu |
| NCT01132664 | Breast carcinoma | BKM120 | PIK3CA, | IHC PTEN | Negative | IHC |
| | | | PTEN | | | HER2/Neu |
| NCT01132664 | Breast carcinoma | BKM120 | PIK3CA, | IHC PTEN | Negative | ISH |
| | | | PTEN | | | HER2/Neu |
| NCT01132664 | Breast carcinoma | BKM120 | PIK3CA, | Seq. PTEN | Mutated | IHC |
| | | | PTEN | | | HER2/Neu |
| NCT01132664 | Breast carcinoma | BKM120 | PIK3CA, | Seq. PTEN | Mutated | ISH |
| | | | PTEN | | | HER2/Neu |
| NCT01141244 | All | temsirolimus | PIK3CA, | Seq. | Mutated \| | |
| | | | PTEN | PIK3CA | exon20 | |
| NCT01141244 | All | temsirolimus | PIK3CA, | Seq. PTEN | Mutated | |
| | | | PTEN | | | |
| NCT01143402 | Uveal Melanoma | AZD6244 | GNAQ, | Seq. | Mutated | |
| | | | GNA11 | GNAQ | | |
| NCT01143402 | Uveal Melanoma | AZD6244 | GNAQ, | Seq. | Mutated | |
| | | | GNA11 | GNA11 | | |
| NCT01148849 | Breast carcinoma, | MGAH22 | Her2 | ISH | Amplified \| | |
| | Gastric | | | Her2/Neu | Equiv. | |
| | adenocarcinoma, | | | | High | |
| | Non epithelial | | | | | |
| | ovarian cancer | | | | | |
| | (non-EOC), | | | | | |
| | Ovarian surface | | | | | |
| | epithelial | | | | | |
| | carcinomas, BAC, | | | | | |
| | NSCLC | | | | | |
| NCT01148849 | Breast carcinoma, | MGAH22 | Her2 | IHC | Positive | |
| | Gastric | | | Her2/Neu | | |
| | adenocarcinoma, | | | | | |
| | Non epithelial | | | | | |
| | ovarian cancer | | | | | |
| | (non-EOC), | | | | | |
| | Ovarian surface | | | | | |
| | epithelial | | | | | |
| | carcinomas, BAC, | | | | | |
| | NSCLC | | | | | |
| NCT01158651 | Glioblastoma | everolimus | PIK3CA, | Seq. | Mutated \| | |
| | | | PTEN | PIK3CA | exon20 | |
| NCT01158651 | Glioblastoma | everolimus | PIK3CA, | Seq. PTEN | Mutated | |
| | | | PTEN | | | |
| NCT01174199 | Prostatic | temsirolimus | PIK3CA, | Seq. | Mutated \| | |
| | Adenocarcinoma | | PTEN | PIK3CA | exon20 | |
| NCT01174199 | Prostatic | temsirolimus | PIK3CA, | Seq. PTEN | Mutated | |
| | Adenocarcinoma | | PTEN | | | |
| NCT01182168 | All | everolimus | PIK3CA, | Seq. | Mutated \| | |
| | | | PTEN | PIK3CA | exon20 | |
| NCT01182168 | All | everolimus | PIK3CA, | Seq. PTEN | Mutated | |
| | | | PTEN | | | |
| NCT01187199 | All | temsirolimus | PIK3CA, | Seq. | Mutated \| | |
| | | | PTEN | PIK3CA | exon20 | |
| NCT01187199 | All | temsirolimus | PIK3CA, | Seq. PTEN | Mutated | |
| | | | PTEN | | | |
| NCT01191697 | Gastric | bevacizumab | VHL | Seq. VHL | Mutated | |
| | adenocarcinoma, | | | | | |
| | Esophageal and | | | | | |
| | Esophagogastric | | | | | |
| | Junction | | | | | |
| | Carcinoma | | | | | |
| NCT01191697 | Gastric | bevacizumab | VHL | Seq. VHL | Mutated | IHC Her2 |
| | adenocarcinoma, | | | | | |
| | Esophageal and | | | | | |
| | Esophagogastric | | | | | |
| | Junction | | | | | |
| | Carcinoma | | | | | |
| NCT01191697 | Gastric | bevacizumab | VHL | Seq. VHL | Mutated | ISH |
| | adenocarcinoma, | | | | | HER2/Neu |
| | Esophageal and | | | | | |
| | Esophagogastric | | | | | |
| | Junction | | | | | |
| | Carcinoma | | | | | |
| NCT01194869 | Breast carcinoma | sorafenib | VHL | Seq. VHL | Mutated | IHC ER |
| NCT01194869 | Breast carcinoma | sorafenib | VHL | Seq. VHL | Mutated | IHC ER |
| NCT01195922 | Head and neck | rapamycin | PIK3CA, | Seq. | Mutated \| | |
| | Squamous | | PTEN | PIK3CA | exon20 | |
| | Carcinoma | | | | | |
| NCT01195922 | Head and neck | rapamycin | PIK3CA, | Seq. PTEN | Mutated | |
| | Squamous | | PTEN | | | |
| | Carcinoma | | | | | |
| NCT01196429 | Ovarian Surface | temsirolimus | PIK3CA, | Seq. | Mutated \| | |
| | Epithelial | | PTEN | PIK3CA | exon20 | |
| | Carcinomas | | | | | |
| NCT01196429 | Ovarian Surface | temsirolimus | PIK3CA, | Seq. PTEN | Mutated | |
| | Epithelial | | PTEN | | | |
| | Carcinomas | | | | | |
| NCT01204099 | BAC, NSCLC, | PX-866 | PIK3CA, | Seq. | Mutated \| | |
| | Head and neck | | PTEN | PIK3CA | exon20 | |
| | Squamous | | | | | |
| | Carcinoma | | | | | |
| NCT01204099 | BAC, NSCLC, | PX-866 | PIK3CA, | IHC PTEN | Negative | |
| | Head and neck | | PTEN | | | |
| | Squamous | | | | | |
| | Carcinoma | | | | | |
| NCT01204099 | BAC, NSCLC, | PX-866 | PIK3CA, | Seq. PTEN | Mutated | |
| | Head and neck | | PTEN | | | |
| | Squamous | | | | | |
| | Carcinoma | | | | | |
| NCT01206530 | Colorectal | bevacizumab | VHL | Seq. VHL | Mutated | |
| | Adenocarcinoma | | | | | |
| NCT01212822 | Gastric | bevacizumab | VHL | Seq. VHL | Mutated | |
| | adenocarcinoma, | | | | | |
| | Esophageal and | | | | | |
| | Esophagogastric | | | | | |
| | Junction | | | | | |
| | Carcinoma | | | | | |
| NCT01218555 | All | everolimus | PIK3CA, | Seq. | Mutated \| | |
| | | | PTEN | PIK3CA | exon20 | |
| NCT01218555 | All | everolimus | PIK3CA, | Seq. PTEN | Mutated | |
| | | | PTEN | | | |
| NCT01219699 | All | BYL719 | PIK3CA, | Seq. | Mutated \| | |
| | | | PTEN | PIK3CA | exon20 | |
| NCT01219699 | All | BYL719 | PIK3CA, | IHC PTEN | Negative | |
| | | | PTEN | | | |
| NCT01219699 | All | BYL719 | PIK3CA, | Seq. PTEN | Mutated | |
| | | | PTEN | | | |
| NCT01222715 | Soft tissue tumors | temsirolimus | PIK3CA, | Seq. | Mutated \| | |
| | | | PTEN | PIK3CA | exon20 | |
| NCT01222715 | Soft tissue tumors | temsirolimus | PIK3CA, | Seq. PTEN | Mutated | |
| | | | PTEN | | | |
| NCT01231399 | Gastric | everolimus | PIK3CA, | Seq. | Mutated \| | |
| | Adenocarcinoma, | | PTEN | PIK3CA | exon20 | |
| | Esophageal and | | | | | |
| | Esophagogastric | | | | | |
| | Junction | | | | | |
| | Carcinoma | | | | | |
| NCT01231399 | Gastric | everolimus | PIK3CA, | Seq. PTEN | Mutated | |
| | Adenocarcinoma, | | PTEN | | | |
| | Esophageal and | | | | | |
| | Esophagogastric | | | | | |
| | Junction | | | | | |
| | Carcinoma | | | | | |
| NCT01231594 | All | GSK2118436 | BRAF | Seq. BRAF | Mutated \| | |
| | | | | | V600E \| | |
| | | | | | V600K | |
| NCT01231594 | All | GSK2118436 | BRAF | PCR BRAF | V600E | |
| NCT01235897 | All | MK2206 | PIK3CA, | Seq. | Mutated \| | IHC |
| | | | PTEN | PIK3CA | exon20 | HER2/Neu |
| NCT01235897 | All | MK2206 | PIK3CA, | IHC PTEN | Negative | IHC |
| | | | PTEN | | | HER2/Neu |
| NCT01235897 | All | MK2206 | PIK3CA, | Seq. | Mutated \| | ISH |
| | | | PTEN | PIK3CA | exon20 | HER2/Neu |
| NCT01235897 | All | MK2206 | PIK3CA, | IHC PTEN | Negative | ISH |
| | | | PTEN | | | HER2/Neu |
| NCT01235897 | All | MK2206 | PIK3CA, | Seq. PTEN | Mutated | IHC |
| | | | PTEN | | | HER2/Neu |
| NCT01235897 | All | MK2206 | PIK3CA, | Seq. PTEN | Mutated | ISH |
| | | | PTEN | | | HER2/Neu |
| NCT01245205 | All | MK2206 | PIK3CA, | Seq. | Mutated \| | |
| | | | PTEN | PIK3CA | exon20 | |
| NCT01245205 | All | MK2206 | PIK3CA, | IHC PTEN | Negative | |
| | | | PTEN | | | |
| NCT01245205 | All | MK2206 | PIK3CA, | Seq. PTEN | Mutated | |
| | | | PTEN | | | |
| NCT01248247 | BAC, NSCLC, | AZD6244 | KRAS, | Seq. KRAS | Mutated \| | |
| | Lung Small Cell | | NRAS, | | G13D | |
| | Cancer (SCLC) | | BRAF | | | |
| NCT01248247 | BAC, NSCLC, | AZD6244 | KRAS, | Seq. BRAF | Mutated \| | |
| | Lung Small Cell | | NRAS, | | V600E \| | |
| | Cancer (SCLC) | | BRAF | | V600K | |
| NCT01248247 | BAC, NSCLC, | AZD6244 | KRAS, | Seq. NRAS | Mutated | |
| | Lung Small Cell | | NRAS, | | | |
| | Cancer (SCLC) | | BRAF | | | |
| NCT01251861 | Prostatic | MK2206 | PIK3CA, | Seq. | Mutated \| | |
| | adenocarcinoma | | PTEN | PIK3CA | exon20 | |
| NCT01251861 | Prostatic | MK2206 | PIK3CA, | IHC PTEN | Negative | |
| | adenocarcinoma | | PTEN | | | |
| NCT01251861 | Prostatic | MK2206 | PIK3CA, | Seq. PTEN | Mutated | |
| | adenocarcinoma | | PTEN | | | |
| NCT01252251 | Uveal Melanoma | everolimus | PIK3CA, | Seq. | Mutated \| | |
| | | | PTEN | PIK3CA | exon20 | |
| NCT01252251 | Uveal Melanoma | everolimus | PIK3CA, | Seq. PTEN | Mutated | |
| | | | PTEN | | | |
| NCT01256268 | Female Genital | ridaforolimus | PIK3CA, | Seq. PTEN | Mutated | |
| | Tract Malignancy, | | PTEN | | | |
| | Ovarian Surface | | | | | |
| | Epithelial | | | | | |
| | Carcinomas | | | | | |
| NCT01256268 | Female Genital | ridaforolimus | PIK3CA, | Seq. | Mutated \| | |
| | Tract Malignancy, | | PTEN | PIK3CA | exon20 | |
| | Ovarian Surface | | | | | |
| | Epithelial | | | | | |
| | Carcinomas | | | | | |
| NCT01256385 | Head and neck | temsirolimus | PIK3CA, | Seq. | Mutated \| | |
| | Squamous | | PTEN | PIK3CA | exon20 | |
| | Carcinoma | | | | | |
| NCT01256385 | Head and neck | temsirolimus | PIK3CA, | Seq. PTEN | Mutated | |
| | Squamous | | PTEN | | | |
| | Carcinoma | | | | | |
| NCT01270321 | Thyroid | everolimus | PIK3CA, | Seq. | Mutated \| | |
| | Carcinoma | | PTEN | PIK3CA | exon20 | |
| NCT01270321 | Thyroid | everolimus | PIK3CA, | Seq. PTEN | Mutated | |
| | Carcinoma | | PTEN | | | |
| NCT01276210 | All | sorafenib | BRAF | Seq. BRAF | Mutated \| | |
| | | | | | V600E \| | |
| | | | | | V600K | |
| NCT01276210 | All | sorafenib | BRAF | PCR BRAF | V600E | |
| NCT01277757 | Breast carcinoma | MK2206 | PIK3CA, | Seq. | Mutated \| | |
| | | | PTEN | PIK3CA | exon20 | |
| NCT01277757 | Breast carcinoma | MK2206 | PIK3CA, | IHC PTEN | Negative | |
| | | | PTEN | | | |
| NCT01277757 | Breast carcinoma | MK2206 | PIK3CA, | Seq. PTEN | Mutated | |
| | | | PTEN | | | |
| NCT01279681 | Colorectal | bevacizumab | VHL | Seq. VHL | Mutated | |
| | Adenocarcinoma | | | | | |
| NCT01281163 | Breast carcinoma | MK2206 | PIK3CA, | Seq. | Mutated \| | IHC |
| | | | PTEN | PIK3CA | exon20 | HER2/Neu |
| NCT01281163 | Breast carcinoma | MK2206 | PIK3CA, | Seq. | Mutated \| | ISH |
| | | | PTEN | PIK3CA | exon20 | Her2/Neu |
| NCT01281163 | Breast carcinoma | MK2206 | PIK3CA, | IHC PTEN | Negative | IHC |
| | | | PTEN | | | HER2/Neu |
| NCT01281163 | Breast carcinoma | MK2206 | PIK3CA, | IHC PTEN | Negative | ISH |
| | | | PTEN | | | Her2/Neu |
| NCT01281163 | Breast carcinoma | MK2206 | PIK3CA, | Seq. PTEN | Mutated | IHC |
| | | | PTEN | | | HER2/Neu |
| NCT01281163 | Breast carcinoma | MK2206 | PIK3CA, | Seq. PTEN | Mutated | ISH |
| | | | PTEN | | | Her2/Neu |
| NCT01281514 | Ovarian Surface | everolimus | PIK3CA, | Seq. | Mutated \| | |
| | Epithelial | | PTEN | PIK3CA | exon20 | |
| | Carcinomas | | | | | |
| NCT01281514 | Ovarian Surface | everolimus | PIK3CA, | Seq. PTEN | Mutated | |
| | Epithelial | | PTEN | | | |
| | Carcinomas | | | | | |
| NCT01283789 | Breast Carcinoma | everolimus | Her2 | IHC Her2 | Positive | |
| NCT01283789 | Breast Carcinoma | everolimus | Her2 | ISH Her2 | Amplified | |
| NCT01283789 | Breast Carcinoma | everolimus | Her2 | ISH Her2 | Equiv. | |
| | | | | | High | |
| NCT01297452 | All | BKM120 | PIK3CA, | Seq. | Mutated \| | |
| | | | PTEN | PIK3CA | exon20 | |
| NCT01297452 | All | BKM120 | PIK3CA, | IHC PTEN | Negative | |
| | | | PTEN | | | |
| NCT01297452 | All | BKM120 | PIK3CA, | Seq. PTEN | Mutated | |
| | | | PTEN | | | |
| NCT01297491 | BAC, NSCLC | BKM120 | PIK3CA, | Seq. | Mutated \| | |
| | | | PTEN | PIK3CA | exon20 | |
| NCT01297491 | BAC, NSCLC | BKM120 | PIK3CA, | IHC PTEN | Negative | |
| | | | PTEN | | | |
| NCT01297491 | BAC, NSCLC | BKM120 | PIK3CA, | Seq. PTEN | Mutated | |
| | | | PTEN | | | |
| NCT01298570 | Colorectal | regorafenib | KRAS, | Seq. KRAS | Mutated \| | |
| | Adenocarcinoma | | BRAF | | G13D | |
| | | | | | Mutated \| | |
| NCT01298570 | Colorectal | regorafenib | KRAS, | Seq. BRAF | V600E \| | |
| | Adenocarcinoma | | BRAF | | V600K | |
| NCT01298570 | Colorectal | regorafenib | VHL | Seq. VHL | Mutated | |
| | Adenocarcinoma | | | | | |
| NCT01300429 | BAC, NSCLC | crizotinib | ALK | ISH ALK | Positive | |
| NCT01300962 | Breast carcinoma | BKM120, | PIK3CA, | Seq. | Mutated \| | |
| | | BEZ235 | PTEN | PIK3CA | exon20 | |
| NCT01300962 | Breast carcinoma | BKM120, | PIK3CA, | IHC PTEN | Negative | |
| | | BEZ235 | PTEN | | | |
| NCT01300962 | Breast carcinoma | BKM120, | PIK3CA, | Seq. PTEN | Mutated | |
| | | BEZ235 | PTEN | | | |
| NCT01301716 | All | GDC-0980 | PIK3CA, | Seq. | Mutated \| | |
| | | | PTEN | PIK3CA | exon20 | |
| NCT01301716 | All | GDC-0980 | PIK3CA, | IHC PTEN | Negative | |
| | | | PTEN | | | |
| NCT01301716 | All | GDC-0980 | PIK3CA, | Seq. PTEN | Mutated | |
| | | | PTEN | | | |
| NCT01304602 | Colorectal | BKM120 | PIK3CA, | Seq. | Mutated \| | |
| | adenocarcinoma | | PTEN | PIK3CA | exon20 | |
| NCT01304602 | Colorectal | BKM120 | PIK3CA, | IHC PTEN | Negative | |
| | adenocarcinoma | | PTEN | | | |
| NCT01304602 | Colorectal | BKM120 | PIK3CA, | Seq. PTEN | Mutated | |
| | adenocarcinoma | | PTEN | | | |
| NCT01305941 | Breast Carcinoma | everolimus | Her2 | IHC Her2 | Positive | |
| NCT01305941 | Breast Carcinoma | everolimus | Her2 | ISH Her2 | Amplified | |
| NCT01305941 | Breast Carcinoma | everolimus | Her2 | ISH Her2 | Equiv. | |
| | | | | | High | |
| NCT01306045 | BAC, NSCLC, | AZD6244 | KRAS, | Seq. KRAS | Mutated \| | |
| | Thymic Carcinoma, | | NRAS, | | G13D | |
| | Lung Small Cell | | BRAF | | | |
| | Cancer (SCLC) | | | | | |
| NCT01306045 | BAC, NSCLC, | AZD6244 | KRAS, | Seq. BRAF | Mutated \| | |
| | Thymic Carcinoma, | | NRAS, | | V600E \| | |
| | Lung Small Cell | | BRAF | | V600K | |
| | Cancer (SCLC) | | | | | |
| NCT01306045 | BAC, NSCLC, | AZD6244 | KRAS, | Seq. NRAS | Mutated | |
| | Thymic Carcinoma, | | NRAS, | | | |
| | Lung Small Cell | | BRAF | | | |
| | Cancer (SCLC) | | | | | |
| NCT01307631 | Female | MK2206 | PIK3CA, | Seq. | Mutated \| | |
| | genital tract | | PTEN | PIK3CA | exon20 | |
| | malignancy | | | | | |
| NCT01307631 | Female | MK2206 | PIK3CA, | IHC PTEN | Negative | |
| | genital tract | | PTEN | | | |
| | malignancy | | | | | |
| NCT01307631 | Female | MK2206 | PIK3CA, | Seq. PTEN | Mutated | |
| | genital tract | | PTEN | | | |
| | malignancy | | | | | |
| NCT01313039 | Breast Carcinoma | AZD6244 | KRAS, | Seq. KRAS | Mutated \| | IHC ER |
| | | | NRAS, | | G13D | |
| | | | BRAF | | | |
| NCT01313039 | Breast Carcinoma | AZD6244 | KRAS, | Seq. BRAF | Mutated \| | IHC ER |
| | | | NRAS, | | V600E \| | |
| | | | BRAF | | V600K | |
| NCT01313039 | Breast Carcinoma | AZD6244 | KRAS, | Seq. NRAS | Mutated | IHC ER |
| | | | NRAS, | | | |
| | | | BRAF | | | |
| NCT01319539 | Breast carcinoma | MK2206 | PIK3CA, | Seq. | Mutated \| | |
| | | | PTEN | PIK3CA | exon20 | |
| NCT01319539 | Breast carcinoma | MK2206 | PIK3CA, | IHC PTEN | Negative | |
| | | | PTEN | | | |
| NCT01319539 | Breast carcinoma | MK2206 | PIK3CA, | Seq. PTEN | Mutated | |
| | | | PTEN | | | |
| NCT01320085 | Melanoma | MEK162 | KRAS, | Seq. KRAS | Mutated \| | |
| | | | NRAS, | | G13D | |
| | | | BRAF | | | |
| NCT01320085 | Melanoma | MEK162 | KRAS, | Seq. BRAF | Mutated \| | |
| | | | NRAS, | | V600E \| | |
| | | | BRAF | | V600K | |
| NCT01320085 | Melanoma | MEK162 | KRAS, | Seq. NRAS | Mutated | |
| | | | NRAS, | | | |
| | | | BRAF | | | |
| NCT01322815 | Colorectal | bevacizumab | VHL | Seq. VHL | Mutated | |
| | Adenocarcinoma | | | | | |
| NCT01331135 | All | sirolimus | PIK3CA, | Seq. | Mutated \| | |
| | | | PTEN | PIK3CA | exon20 | |
| NCT01331135 | All | sirolimus | PIK3CA, | Seq. PTEN | Mutated | |
| | | | PTEN | | | |
| NCT01333475 | Colorectal | AZD6244 | KRAS, | Seq. KRAS | Mutated \| | |
| | adenocarcinoma | | NRAS, | | G13D | |
| | | | BRAF | | | |
| NCT01333475 | Colorectal | AZD6244 | KRAS, | Seq. BRAF | Mutated \| | |
| | adenocarcinoma | | NRAS, | | V600E \| | |
| | | | BRAF | | V600K | |
| NCT01333475 | Colorectal | AZD6244 | KRAS, | Seq. NRAS | Mutated | |
| | adenocarcinoma | | NRAS, | | | |
| | | | BRAF | | | |
| NCT01339052 | Glioblastoma | BKM120 | PIK3CA, | Seq. | Mutated \| | |
| | | | PTEN | PIK3CA | exon20 | |
| NCT01339052 | Glioblastoma | BKM120 | PIK3CA, | IHC PTEN | Negative | |
| | | | PTEN | | | |
| NCT01339052 | Glioblastoma | BKM120 | PIK3CA, | Seq. PTEN | Mutated | |
| | | | PTEN | | | |
| NCT01339442 | Breast carcinoma | BKM120 | PIK3CA, | Seq. | Mutated \| | IHC ER |
| | | | PTEN | PIK3CA | exon20 | |
| NCT01339442 | Breast carcinoma | BKM120 | PIK3CA, | IHC PTEN | Negative | IHC ER |
| | | | PTEN | | | |
| NCT01339442 | Breast carcinoma | BKM120 | PIK3CA, | Seq. PTEN | Mutated | IHC ER |
| | | | PTEN | | | |
| NCT01344031 | Breast carcinoma | MK2206 | PIK3CA, | Seq. | Mutated \| | IHC ER |
| | | | PTEN | PIK3CA | exon20 | |
| NCT01344031 | Breast carcinoma | MK2206 | PIK3CA, | IHC PTEN | Negative | IHC ER |
| | | | PTEN | | | |
| NCT01344031 | Breast carcinoma | MK2206 | PIK3CA, | Seq. PTEN | Mutated | IHC ER |
| | | | PTEN | | | |
| NCT01347866 | All | PF-04691502, | PIK3CA, | Seq. | Mutated \| | |
| | | PF-05212384 | PTEN | PIK3CA | exon20 | |
| NCT01347866 | All | PF-04691502, | PIK3CA, | IHC PTEN | Negative | |
| | | PF-05212384 | PTEN | | | |
| NCT01347866 | All | PF-04691502, | PIK3CA, | Seq. PTEN | Mutated | |
| | | PF-05212384 | PTEN | | | |
| NCT01349660 | All | BKM120 | PIK3CA, | Seq. | Mutated \| | |
| | | | PTEN | PIK3CA | exon20 | |
| NCT01349660 | All | BKM120 | PIK3CA, | IHC PTEN | Negative | |
| | | | PTEN | | | |
| NCT01349660 | All | BKM120 | PIK3CA, | Seq. PTEN | Mutated | |
| | | | PTEN | | | |
| NCT01349933 | Head and neck | MK2206 | PIK3CA, | Seq. | Mutated \| | |
| | squamous | | PTEN | PIK3CA | exon20 | |
| | carcinoma | | | | | |
| NCT01349933 | Head and neck | MK2206 | PIK3CA, | IHC PTEN | Negative | |
| | squamous | | PTEN | | | |
| | carcinoma | MK2206 | PIK3CA, | Seq. PTEN | Mutated | |
| NCT01349933 | Head and neck | | PTEN | | | |
| | squamous | | | | | |
| | carcinoma | | | | | |
| NCT01362374 | All | GDC-0068 | PIK3CA, | Seq. | Mutated \| | |
| | | | PTEN | PIK3CA | exon20 | |
| NCT01362374 | All | GDC-0068 | PIK3CA, | IHC PTEN | Negative | |
| | | | PTEN | | | |
| NCT01362374 | All | GDC-0068 | PIK3CA, | Seq. PTEN | Mutated | |
| | | | PTEN | | | |
| NCT01363232 | All | MEK162 | KRAS, | Seq. KRAS | Mutated \| | |
| | | | NRAS, | | G13D | |
| | | | BRAF | | | |
| NCT01363232 | All | MEK162 | KRAS, | Seq. BRAF | Mutated \| | |
| | | | NRAS, | | V600E \| | |
| | | | BRAF | | V600K | |
| NCT01363232 | All | MEK162 | KRAS, | Seq. NRAS | Mutated | |
| | | | NRAS, | | | |
| | | | BRAF | | | |
| NCT01364051 | Melanoma | AZD6244 | KRAS, | Seq. KRAS | Mutated \| | |
| | | | NRAS, | | G13D | |
| | | | BRAF | | | |
| NCT01364051 | Melanoma | AZD6244 | KRAS, | Seq. BRAF | Mutated \| | |
| | | | NRAS, | | V600E \| | |
| | | | BRAF | | V600K | |
| NCT01364051 | Melanoma | AZD6244 | KRAS, | Seq. NRAS | Mutated | |
| | | | NRAS, | | | |
| | | | BRAF | | | |
| NCT01374425 | Colorectal | bevacizumab | VHL | Seq. VHL | Mutated | |
| | Adenocarcinoma | | | | | |
| NCT01375829 | All | temsirolimus | PIK3CA, | Seq. | Mutated \| | |
| | | | PTEN | PIK3CA | exon20 | |
| NCT01375829 | All | temsirolimus | PIK3CA, | Seq. PTEN | Mutated | |
| | | | PTEN | | | |
| NCT01376310 | All | GSK1120212 | KRAS, | Seq. KRAS | Mutated \| | |
| | | | NRAS, | | G13D | |
| | | | BRAF | | | |
| NCT01376310 | All | GSK1120212 | KRAS, | Seq. BRAF | Mutated \| | |
| | | | NRAS, | | V600E \| | |
| | | | BRAF | | V600K | |
| NCT01376310 | All | GSK1120212 | KRAS, | Seq. NRAS | Mutated | |
| | | | NRAS, | | | |
| | | | BRAF | | | |
| NCT01376453 | Colorectal | sorafenib | BRAF | Seq. BRAF | Mutated \| | |
| | Adenocarcinoma | | | | V600E \| | |
| | | | | | V600K | |
| NCT01376453 | Colorectal | sorafenib | BRAF | PCR BRAF | V600E | |
| | Adenocarcinoma | | | | | |
| NCT01385228 | Prostatic | pazopanib | VHL | Seq. VHL | Mutated | |
| | adenocarcinoma | | | | | |
| NCT01385293 | Prostatic | BKM120 | PIK3CA, | Seq. | Mutated \| | |
| | adenocarcinoma | | PTEN | PIK3CA | exon20 | |
| NCT01385293 | Prostatic | BKM120 | PIK3CA, | IHC PTEN | Negative | |
| | adenocarcinoma | | PTEN | | | |
| NCT01385293 | Prostatic | BKM120 | PIK3CA, | Seq. PTEN | Mutated | |
| | adenocarcinoma | | PTEN | | | |
| NCT01386450 | Glioblastoma | AZD6244 | KRAS, | Seq. KRAS | Mutated \| | |
| | | | NRAS, | | G13D | |
| | | | BRAF | | | |
| NCT01386450 | Glioblastoma | AZD6244 | KRAS, | Seq. BRAF | Mutated \| | |
| | | | NRAS, | | V600E \| | |
| | | | BRAF | | V600K | |
| NCT01386450 | Glioblastoma | AZD6244 | KRAS, | Seq. NRAS | Mutated | |
| | | | NRAS, | | | |
| | | | BRAF | | | |
| NCT01390818 | All | MSC1936369B, | GNAQ, | Seq. | Mutated | |
| | | SAR245409 | GNA11 | GNAQ | | |
| NCT01390818 | All | MSC1936369B, | GNAQ, | Seq. | Mutated | |
| | | SAR245409 | GNA11 | GNA11 | | |
| NCT01392521 | All | BAY 86-9766 | KRAS, | Seq. KRAS | Mutated \| | |
| | | | NRAS, | | G13D | |
| | | | BRAF | | | |
| NCT01392521 | All | BAY 86-9766 | KRAS, | Seq. BRAF | Mutated \| | |
| | | | NRAS, | | V600E \| | |
| | | | BRAF | | V600K | |
| NCT01392521 | All | BAY 86-9766 | KRAS, | Seq. NRAS | Mutated | |
| | | | NRAS, | | | |
| | | | BRAF | | | |
| NCT01396148 | Gastrointestinal | Sunitinib | VHL | Seq. VHL | Mutated | |
| | Stromal Tumors | | | | | |
| | (GIST) | | | | | |
| NCT01409200 | Prostatic | axitinib | VHL | Seq. VHL | Mutated | |
| | adenocarcinoma | | | | | |
| NCT01420081 | Female | PF-04691502, | PIK3CA, | Seq. | Mutated \| | |
| | genital tract | PF-05212384 | PTEN | PIK3CA | exon20 | |
| | malignancy | | | | | |
| NCT01420081 | Female | PF-04691502, | PIK3CA, | IHC PTEN | Negative | |
| | genital tract | PF-05212384 | PTEN | | | |
| | malignancy | | | | | |
| NCT01420081 | Female | PF-04691502, | PIK3CA, | Seq. PTEN | Mutated | |
| | genital tract | PF-05212384 | PTEN | | | |
| | malignancy | | | | | |
| NCT01427946 | BAC, NSCLC | everolimus | KRAS | Seq. KRAS | Mutated \| | |
| | | | | | G13D | |
| NCT01434602 | Glioblastoma | sorafenib | BRAF | Seq. BRAF | Mutated \| | |
| | | | | | V600E \| | |
| | | | | | V600K | |
| NCT01434602 | Glioblastoma | sorafenib | BRAF | PCR BRAF | V600E | |
| NCT01437566 | Breast | GDC-0980 | PIK3CA, | Seq. | Mutated \| | |
| | Carcinoma | | PTEN | PIK3CA | exon20 | |
| NCT01437566 | Breast | GDC-0980 | PIK3CA, | IHC PTEN | Negative | |
| | Carcinoma | | PTEN | | | |
| NCT01437566 | Breast | GDC-0980 | PIK3CA, | Seq. PTEN | Mutated | |
| | Carcinoma | | PTEN | | | |
| NCT01441947 | Breast | cabozantinib | VHL | Seq. VHL | Mutated | |
| | Carcinoma | | | | | |
| NCT01449058 | All | BYL719 | PIK3CA, | Seq. | Mutated \| | |
| | | | PTEN | PIK3CA | exon20 | |
| NCT01449058 | All | MEK162 | KRAS, | Seq. KRAS | Mutated \| | |
| | | | NRAS, | | G13D | |
| | | | BRAF | | | |
| NCT01449058 | All | MEK162 | KRAS, | Seq. BRAF | Mutated \| | |
| | | | NRAS, | | V600E \| | |
| | | | BRAF | | V600K | |
| NCT01449058 | All | MEK162 | KRAS, | Seq. NRAS | Mutated | |
| | | | NRAS, | | | |
| | | | BRAF | | | |
| NCT01449058 | All | BYL719 | PIK3CA, | IHC PTEN | Negative | |
| | | | PTEN | | | |
| NCT01449058 | All | BYL719 | PIK3CA, | Seq. PTEN | Mutated | |
| | | | PTEN | | | |
| NCT01450384 | All | sorafenib | BRAF | Seq. BRAF | Mutated \| | |
| | | | | | V600E \| | |
| | | | | | V600K | |
| NCT01450384 | All | sorafenib | BRAF | PCR BRAF | V600E | |
| NCT01465802 | BAC, NSCLC | PF-00299804 | Her2 | ISH | Amplified \| | |
| | | | | Her2/Neu | Equiv. | |
| | | | | | High | |
| NCT01465802 | BAC, NSCLC | PF-00299804 | Her2 | IHC | Positive | |
| | | | | Her2/Neu | | |
| NCT01466244 | Head and neck | cetuximab | PTEN | Seq. PTEN | Mutated | |
| | squamous | | | | | |
| | carcinoma | | | | | |
| NCT01466244 | Head and neck | cetuximab | PTEN | IHC PTEN | Negative | |
| | squamous | | | | | |
| | carcinoma | | | | | |
| NCT01466972 | Breast Carcinoma | Pazopanib | VHL | Seq. VHL | Mutated | |
| NCT01468688 | Gastrointestinal | BKM120 | PIK3CA, | Seq. | Mutated \| | |
| | stromal tumors | | PTEN | PIK3CA | exon20 | |
| | (GIST) | | | | | |
| NCT01468688 | Gastrointestinal | BKM120 | PIK3CA, | IHC PTEN | Negative | |
| | stromal tumors | | PTEN | | | |
| | (GIST) | | | | | |
| NCT01468688 | Gastrointestinal | BKM120 | PIK3CA, | Seq. PTEN | Mutated | |
| | stromal tumors | | PTEN | | | |
| | (GIST) | | | | | |
| NCT01469572 | Neuroendocrine | everolimus | PIK3CA, | Seq. | Mutated \| | |
| | tumors | | PTEN | PIK3CA | exon20 | |
| NCT01469572 | Neuroendocrine | everolimus | PIK3CA, | Seq. PTEN | Mutated | |
| | tumors | | PTEN | | | |
| NCT01470209 | All | BKM120 | PIK3CA, | Seq. | Mutated \| | |
| | | | PTEN | PIK3CA | exon20 | |
| NCT01470209 | All | BKM120 | PIK3CA, | IHC PTEN | Negative | |
| | | | PTEN | | | |
| NCT01470209 | All | BKM120 | PIK3CA, | Seq. PTEN | Mutated | |
| | | | PTEN | | | |
| NCT01471353 | Colorectal | sorafenib | VHL | Seq. VHL | Mutated | |
| | Adenocarcinoma | | | | | |
| NCT01473901 | Glioblastoma | BKM120 | PIK3CA, | Seq. | Mutated \| | |
| | | | PTEN | PIK3CA | exon20 | |
| NCT01473901 | Glioblastoma | BKM120 | PIK3CA, | IHC PTEN | Negative | |
| | | | PTEN | | | |
| NCT01473901 | Glioblastoma | BKM120 | PIK3CA, | Seq. PTEN | Mutated | |
| | | | PTEN | | | |
| NCT01480154 | Prostatic | MK2206 | PIK3CA, | Seq. | Mutated \| | |
| | adenocarcinoma | | PTEN | PIK3CA | exon20 | |
| NCT01480154 | Prostatic | MK2206 | PIK3CA, | IHC PTEN | Negative | |
| | adenocarcinoma | | PTEN | | | |
| NCT01480154 | Prostatic | MK2206 | PIK3CA, | Seq. PTEN | Mutated | |
| | adenocarcinoma | | PTEN | | | |
| NCT01482156 | All | BEZ235 | PIK3CA, | Seq. | Mutated \| | |
| | | | PTEN | PIK3CA | exon20 | |
| NCT01482156 | All | BEZ235 | PIK3CA, | IHC PTEN | Negative | |
| | | | PTEN | | | |
| NCT01482156 | All | BEZ235 | PIK3CA, | Seq. PTEN | Mutated | |
| | | | PTEN | | | |
| NCT01488487 | Liver | everolimus | PIK3CA, | Seq. | Mutated \| | |
| | Hepatocellular | | PTEN | PIK3CA | exon20 | |
| | Carcinoma | | | | | |
| NCT01488487 | Liver | everolimus | PIK3CA, | Seq. PTEN | Mutated | |
| | Hepatocellular | | PTEN | | | |
| | Carcinoma | | | | | |
| NCT01490749 | Esophageal and | everolimus | PIK3CA, | Seq. | Mutated \| | |
| | Esophagogastric | | PTEN | PIK3CA | exon20 | |
| | Junction | | | | | |
| | Carcinoma | | | | | |
| NCT01490749 | Esophageal and | everolimus | PIK3CA, | Seq. PTEN | Mutated | |
| | Esophagogastric | | PTEN | | | |
| | Junction | | | | | |
| | Carcinoma | | | | | |
| NCT01490866 | Colorectal | Axitinib | VHL | Seq. VHL | Mutated | |
| | Adenocarcinoma | | | | | |
| NCT01495247 | Breast carcinoma | BEZ235 | PIK3CA, | Seq. | Mutated \| | IHC |
| | | | PTEN | PIK3CA | exon20 | HER2/Neu |
| NCT01495247 | Breast carcinoma | BEZ235 | PIK3CA, | Seq. | Mutated \| | ISH |
| | | | PTEN | PIK3CA | exon20 | HER2/Neu |
| NCT01495247 | Breast carcinoma | BEZ235 | PIK3CA, | IHC PTEN | Negative | IHC |
| | | | PTEN | | | HER2/Neu |
| NCT01495247 | Breast carcinoma | BEZ235 | PIK3CA, | IHC PTEN | Negative | ISH |
| | | | PTEN | | | HER2/Neu |
| NCT01495247 | Breast carcinoma | BEZ235 | PIK3CA, | Seq. PTEN | Mutated | IHC |
| | | | PTEN | | | HER2/Neu |
| NCT01495247 | Breast carcinoma | BEZ235 | PIK3CA, | Seq. PTEN | Mutated | ISH |
| | | | PTEN | | | HER2/Neu |
| NCT01499160 | Breast Carcinoma | everolimus | ER, PR | IHC ER | Positive | |
| NCT01499160 | Breast Carcinoma | everolimus | ER, PR | IHC PR | Positive | |
| NCT01508104 | All | BEZ235 | PIK3CA, | Seq. | Mutated \| | |
| | | | PTEN | PIK3CA | exon20 | |
| NCT01508104 | All | BEZ235 | PIK3CA, | IHC PTEN | Negative | |
| | | | PTEN | | | |
| NCT01508104 | All | BEZ235 | PIK3CA, | Seq. PTEN | Mutated | |
| | | | PTEN | | | |
| NCT01512251 | Melanoma | BKM120 | PIK3CA, | Seq. | Mutated \| | |
| | | | PTEN | PIK3CA | exon20 | |
| NCT01512251 | Melanoma | BKM120 | PIK3CA, | IHC PTEN | Negative | |
| | | | PTEN | | | |
| NCT01512251 | Melanoma | BKM120 | PIK3CA, | Seq. PTEN | Mutated | |
| | | | PTEN | | | |
| NCT01516216 | Colorectal | bevacizumab | VHL | Seq. VHL | Mutated | |
| | Adenocarcinoma | | | | | |
| NCT01519414 | Prostatic | ARQ 197 | cMET | ISH CMET | Amplified | |
| | adenocarcinoma | | | | | |
| NCT01519414 | Prostatic | ARQ 197 | cMET | IHC CMET | Positive | |
| | adenocarcinoma | | | | | |
| NCT01522768 | Gastric | afatinib | Her2 | ISH | Amplified \| | |
| | adenocarcinoma, | | | Her2/Neu | Equiv. | |
| | Esophageal and | | | | High | |
| | Esophagogastric | | | | | |
| | Junction | | | | | |
| | Carcinoma | | | | | |
| NCT01522768 | Gastric | afatinib | Her2 | IHC | Positive | |
| | adenocarcinoma, | | | Her2/Neu | | |
| | Esophageal and | | | | | |
| | Esophagogastric | | | | | |
| | Junction | | | | | |
| | Carcinoma | | | | | |
| NCT01524783 | Neuroendocrine | everolimus | PIK3CA, | Seq. | Mutated \| | |
| | tumors | | PTEN | PIK3CA | exon20 | |
| NCT01524783 | Neuroendocrine | everolimus | PIK3CA, | Seq. PTEN | Mutated | |
| | tumors | | PTEN | | | |
| NCT01529593 | All | temsirolimus, | PIK3CA, | Seq. | Mutated \| | |
| | | metformin | PTEN | PIK3CA | exon20 | |
| NCT01529593 | All | temsirolimus, | PIK3CA, | Seq. PTEN | Mutated | |
| | | metformin | PTEN | | | |
| NCT01531361 | All | vemurafenib, | BRAF | Seq. BRAF | Mutated \| | |
| | | sorafenib | | | V600E \| | |
| | | | | | V600K | |
| NCT01531361 | All | vemurafenib, | BRAF | PCR BRAF | V600E | |
| | | sorafenib | | | | |
| NCT01536054 | Ovarian Surface | sirolimus | PIK3CA, | Seq. | Mutated \| | |
| | Epithelial | | PTEN | PIK3CA | exon20 | |
| | Carcinomas | | | | | |
| NCT01536054 | Ovarian Surface | sirolimus | PIK3CA, | Seq. PTEN | Mutated | |
| | Epithelial | | PTEN | | | |
| | Carcinomas | | | | | |
| NCT01538680 | Colorectal | regorafenib | KRAS, | Seq. KRAS | Mutated \| | |
| | Adenocarcinoma | | BRAF | | G13D | |
| NCT01538680 | Colorectal | regorafenib | KRAS, | Seq. BRAF | Mutated \| | |
| | Adenocarcinoma | | BRAF | | V600E \| | |
| | | | | | V600K | |
| NCT01540253 | All | BKM120 | PIK3CA, | Seq. | Mutated \| | |
| | | | PTEN | PIK3CA | exon20 | |
| NCT01540253 | All | BKM120 | PIK3CA, | IHC PTEN | Negative | |
| | | | PTEN | | | |
| NCT01540253 | All | BKM120 | PIK3CA, | Seq. PTEN | Mutated | |
| | | | PTEN | | | |
| NCT01542996 | Breast | ARQ 197 | cMET | ISH CMET | Amplified | IHC ER |
| | Carcinoma | | | | | |
| NCT01542996 | Breast | ARQ 197 | cMET | IHC CMET | Positive | IHC ER |
| | Carcinoma | | | | | |
| NCT01542996 | Breast | ARQ 197 | cMET | ISH CMET | Amplified | IHC ER |
| | Carcinoma | | | | | |
| NCT01542996 | Breast | ARQ 197 | cMET | IHC CMET | Positive | IHC ER |
| | Carcinoma | | | | | |
| NCT01543698 | All | MEK162 | KRAS, | Seq. KRAS | Mutated \| | |
| | | | NRAS, | | G13D | |
| | | | BRAF | | | |
| NCT01543698 | All | MEK162 | KRAS, | Seq. BRAF | Mutated \| | |
| | | | NRAS, | | V600E \| | |
| | | | BRAF | | V600K | |
| NCT01543698 | All | MEK162 | KRAS, | Seq. NRAS | Mutated | |
| | | | NRAS, | | | |
| | | | BRAF | | | |
| NCT01545947 | BAC, NSCLC | CC-223 | PIK3CA, | Seq. | Mutated \| | |
| | | | PTEN | PIK3CA | exon20 | |
| NCT01545947 | BAC, NSCLC | CC-223 | PIK3CA, | Seq. PTEN | Mutated | |
| | | | PTEN | | | |
| NCT01548144 | All | crizotinib | ALK | ISH ALK | Positive | |
| NCT01548144 | All | crizotinib | ROS1 | ISH ROS1 | Positive | |
| NCT01548807 | Prostatic | everolimus | PIK3CA, | Seq. | Mutated \| | |
| | Adenocarcinoma | | PTEN | PIK3CA | exon20 | |
| NCT01548807 | Prostatic | everolimus | PIK3CA, | Seq. PTEN | Mutated | |
| | Adenocarcinoma | | PTEN | | | |
| NCT01550380 | Female | BKM120 | PIK3CA, | Seq. | Mutated \| | |
| | genital tract | | PTEN | PIK3CA | exon20 | |
| | malignancy | | | | | |
| NCT01550380 | Female | BKM120 | PIK3CA, | IHC PTEN | Negative | |
| | genital tract | | PTEN | | | |
| | malignancy | | | | | |
| NCT01550380 | Female | BKM120 | PIK3CA, | Seq. PTEN | Mutated | |
| | genital tract | | PTEN | | | |
| | malignancy | | | | | |
| NCT01552434 | All | temsirolimus | PIK3CA, | Seq. | Mutated \| | |
| | | | PTEN | PIK3CA | exon20 | |
| NCT01552434 | All | temsirolimus | PIK3CA, | Seq. PTEN | Mutated | |
| | | | PTEN | | | |
| NCT01553851 | Head and neck | GSK1120212 | KRAS, | Seq. KRAS | Mutated \| | |
| | squamous | | NRAS, | | G13D | |
| | carcinoma | | BRAF | | | |
| NCT01553851 | Head and neck | GSK1120212 | KRAS, | Seq. BRAF | Mutated \| | |
| | squamous | | NRAS, | | V600E \| | |
| | carcinoma | | BRAF | | V600K | |
| NCT01553851 | Head and neck | GSK1120212 | KRAS, | Seq. NRAS | Mutated | |
| | squamous | | NRAS, | | | |
| | carcinoma | | BRAF | | | |
| NCT01553942 | BAC, NSCLC | afatinib | EGFR | Seq. EGFR | Activating | |
| | | | | | Mutation \| | |
| | | | | | Exon 21 | |
| | | | | | L858R \| | |
| | | | | | Exon 19 del | |
| NCT01562275 | All | GDC-0068 | PIK3CA, | Seq. | Mutated \| | |
| | | | PTEN | PIK3CA | exon20 | |
| NCT01562275 | All | GDC-0068 | PIK3CA, | IHC PTEN | Negative | |
| | | | PTEN | | | |
| NCT01562275 | All | GDC-0068 | PIK3CA, | Seq. PTEN | Mutated | |
| | | | PTEN | | | |
| NCT01562899 | Colorectal | MEK162 | KRAS, | Seq. KRAS | Mutated \| | |
| | adenocarcinoma, | | NRAS, | | G13D | |
| | Melanoma, | | BRAF | | | |
| | Pancreatic | | | | | |
| | adenocarcinoma | | | | | |
| NCT01562899 | Colorectal | MEK162 | KRAS, | Seq. BRAF | Mutated \| | |
| | adenocarcinoma, | | NRAS, | | V600E \| | |
| | Melanoma, | | BRAF | | V600K | |
| | Pancreatic | | | | | |
| | adenocarcinoma | | | | | |
| NCT01562899 | Colorectal | MEK162 | KRAS, | Seq. NRAS | Mutated | |
| | adenocarcinoma, | | NRAS, | | | |
| | Melanoma, | | BRAF | | | |
| | Pancreatic | | | | | |
| | adenocarcinoma | | | | | |
| NCT01563354 | Neuroendocrine | everolimus | PIK3CA, | Seq. | Mutated \| | |
| | tumors | | PTEN | PIK3CA | exon20 | |
| NCT01563354 | Neuroendocrine | everolimus | PIK3CA, | Seq. PTEN | Mutated | |
| | tumors | | PTEN | | | |
| NCT01567930 | Liver | temsirolimus | PIK3CA, | Seq. | Mutated \| | |
| | Hepatocellular | | PTEN | PIK3CA | exon20 | |
| | Carcinoma | | | | | |
| NCT01567930 | Liver | temsirolimus | PIK3CA, | Seq. PTEN | Mutated | |
| | Hepatocellular | | PTEN | | | |
| | Carcinoma | | | | | |
| NCT01571024 | Colorectal | BKM120 | PIK3CA, | Seq. | Mutated \| | |
| | adenocarcinoma, | | PTEN | PIK3CA | exon20 | |
| | Pancreatic | | | | | |
| | adenocarcinoma | | | | | |
| NCT01571024 | Colorectal | BKM120 | PIK3CA, | IHC PTEN | Negative | |
| | adenocarcinoma, | | PTEN | | | |
| | Pancreatic | | | | | |
| | adenocarcinoma | | | | | |
| NCT01571024 | Colorectal | BKM120 | PIK3CA, | Seq. PTEN | Mutated | |
| | adenocarcinoma, | | PTEN | | | |
| | Pancreatic | | | | | |
| | adenocarcinoma | | | | | |
| NCT01571284 | Colorectal | aflibercept | VHL | Seq. VHL | Mutated | |
| | Adenocarcinoma | | | | | |
| NCT01576406 | All | crizotinib | ALK | ISH ALK | Positive | |
| NCT01576666 | All | BKM120 | PIK3CA, | Seq. | Mutated \| | |
| | | | PTEN | PIK3CA | exon20 | |
| NCT01576666 | All | BKM120 | PIK3CA, | IHC PTEN | Negative | |
| | | | PTEN | | | |
| NCT01576666 | All | BKM120 | PIK3CA, | Seq. PTEN | Mutated | |
| | | | PTEN | | | |
| NCT01579994 | BAC, NSCLC | crizotinib | ALK | ISH ALK | Positive | |
| NCT01582191 | All | everolimus | PIK3CA, | Seq. | Mutated \| | |
| | | | PTEN | PIK3CA | exon20 | |
| NCT01582191 | All | everolimus | PIK3CA, | Seq. PTEN | Mutated | |
| | | | PTEN | | | |
| NCT01587040 | All | SAR245409 | PIK3CA, | Seq. | Mutated \| | |
| | | | PTEN | PIK3CA | exon20 | |
| NCT01587040 | All | SAR245409 | PIK3CA, | IHC PTEN | Negative | |
| | | | PTEN | | | |
| NCT01587040 | All | SAR245409 | PIK3CA, | Seq. PTEN | Mutated | |
| | | | PTEN | | | |
| NCT01587352 | Uveal Melanoma | vorinostat | GNAQ, | Seq. | Mutated | |
| | | | GNA11 | GNAQ | | |
| NCT01587352 | Uveal Melanoma | vorinostat | GNAQ, | Seq. | Mutated | |
| | | | GNA11 | GNA11 | | |
| NCT01596270 | All | SAR245409 | PIK3CA, | Seq. | Mutated \| | |
| | | | PTEN | PIK3CA | exon20 | |
| NCT01596270 | All | SAR245409 | PIK3CA, | IHC PTEN | Negative | |
| | | | PTEN | | | |
| NCT01596270 | All | SAR245409 | PIK3CA, | Seq. PTEN | Mutated | |
| | | | PTEN | | | |
| NCT01661972 | Colorectal | aflibercept | VHL | Seq. VHL | Mutated | |
| | Adenocarcinoma | | | | | |
| NCT01693068 | Melanoma | MSBC1936369B | NRAS | Seq. NRAS | Mutated | |
| NCT01745367 | Breast Carcinoma | tivozanib | VHL | Seq. VHL | Mutated | IHC ER |
| NCT01745367 | Breast Carcinoma | tivozanib | VHL | Seq. VHL | Mutated | IHC ER |
| NCT01456325 | BAC, NSCLC | onartuzumab | cMET | IHC CMET | Positive | Seq. |
| | | | | | | EGFR |
| NCT01662869 | Esophageal and | onartuzumab | cMET | IHC CMET | Positive | IHC |
| | Esophagogastric | | | | | HER2 |
| | Junction | | | | | |
| | Carcinoma | | | | | |
| NCT01121575 | BAC, NSCLC | erizotinib | cMET | Seq. | Mutated | |
| | | | | CMET | | |
| NCT01548144 | All | erizotinib | cMET | Seq. | Mutated | |
| | | | | CMET | | |
| NCT01744652 | All | erizotinib | cMET | Seq. | Mutated | |
| | | | | CMET | | |
| NCT01276041 | Breast Carcinoma | pertuzumab | Her2 | IHC HER2 | Positive | |
| NCT01276041 | Breast Carcinoma | pertuzumab | Her2 | ISH HER2 | Amplified \| | |
| | | | | | Equiv. | |
| | | | | | High | |
| NCT01774786 | Esophageal and | pertuzumab | Her2 | IHC HER2 | Positive | |
| | Esophagogastric | | | | | |
| | Junction | | | | | |
| | Carcinoma, Gastric | | | | | |
| | adenocarcinoma | | | | | |
| NCT01774786 | Esophageal and | pertuzumab | Her2 | ISH HER2 | Amplified \| | |
| | Esophagogastric | | | | Equiv. | |
| | Junction | | | | High | |
| | Carcinoma, Gastric | | | | | |
| | adenocarcinoma | | | | | |
| NCT01565083 | Breast Carcinoma | pertuzumab | Her2 | IHC HER2 | Positive | |
| NCT01565083 | Breast Carcinoma | pertuzumab | Her2 | ISH HER2 | Amplified \| | |
| | | | | | Equiv. | |
| | | | | | High | |
| NCT01491737 | Breast Carcinoma | pertuzumab | Her2 | IHC HER2 | Positive | IHC ER |
| NCT01491737 | Breast Carcinoma | pertuzumab | Her2 | ISH HER2 | Amplified \| | IHC ER |
| | | | | | Equiv. | |
| | | | | | High | |
| NCT01491737 | Breast Carcinoma | pertuzumab | Her2 | IHC HER2 | Positive | IHC PR |
| NCT01491737 | Breast Carcinoma | pertuzumab | Her2 | ISH HER2 | Amplified \| | IHC PR |
| | | | | | Equiv. | |
| | | | | | High | |
| NCT01042379 | Breast Carcinoma | T-DM1, | Her2 | IHC HER2 | Positive | |
| | | pertuzumab | | | | |
| NCT01042379 | Breast Carcinoma | T-DM1, | Her2 | ISH HER2 | Amplified \| | |
| | | pertuzumab | | | Equiv. | |
| | | | | | High | |
| NCT01641939 | Gastric | T-DM1 | Her2 | IHC HER2 | Positive | |
| | adenocarcinoma | | | | | |
| NCT01641939 | Gastric | T-DM1 | Her2 | ISH HER2 | Amplified \| | |
| | adenocarcinoma | | | | Equiv. | |
| | | | | | High | |
| NCT01912963 | Breast Carcinoma | pertuzumab | Her2 | IHC HER2 | Positive | |
| NCT01912963 | Breast Carcinoma | pertuzumab | Her2 | ISH HER2 | Amplified \| | |
| | | | | | Equiv. | |
| | | | | | High | |
| NCT01796197 | Breast Carcinoma | pertuzumab | Her2 | IHC HER2 | Positive | |
| NCT01796197 | Breast Carcinoma | pertuzumab | Her2 | ISH HER2 | Amplified \| | |
| | | | | | Equiv. | |
| | | | | | High | |
| NCT01855828 | Breast Carcinoma | pertuzumab | Her2 | IHC HER2 | Positive | |
| NCT01855828 | Breast Carcinoma | pertuzumab | Her2 | ISH HER2 | Amplified \| | |
| | | | | | Equiv. | |
| | | | | | High | |
| NCT01904903 | Breast Carcinoma | pertuzumab, T- | Her2 | IHC HER2 | Positive | |
| | | DM1 | | | | |
| NCT01904903 | Breast Carcinoma | pertuzumab, T- | Her2 | ISH HER2 | Amplified \| | |
| | | DM1 | | | Equiv. | |
| | | | | | High | |
| NCT01480479 | Glioblastoma | Rindopepimut | EGFRvIII | FA | Present | |
| | | | | EGFRvIII | | |
| NCT01498328 | Glioblastoma | Rindopepimut | EGFRvIII | FA | Present | |
| | | | | EGFRvIII | | |
| NCT01800695 | Glioblastoma | ABT-414 | EGFRvIII | FA | Present | |
| | | | | EGFRvIII | | |
| NCT01475006 | Glioblastoma | AMG 595 | EGFRvIII | FA | Present | |
| | | | | EGFRvIII | | |
| NCT01257594 | Glioblastoma | erlotinib | EGFRvIII | FA | Present | |
| | | | | EGFRvIII | | |
| NCT00301418 | Glioblastoma | erlotinib | EGFRvIII | FA | Present | |
| | | | | EGFRvIII | | |
| NCT01465802 | BAC, NSCLC | dacomitinib | EGFRvIII | FA | Present | |
| | | | | EGFRvIII | | |
| NCT01858389 | BAC, NSCLC | dacomitinib | EGFRvIII | FA | Present | |
| | | | | EGFRvIII | | |
| NCT01454596 | Glioblastoma | Anti-EGFRvIII | EGFRvIII | FA | Present | |
| | | CAR | | EGFRvIII | | |
| NCT01112527 | Glioblastoma | PF-00299804 | EGFRvIII | FA | Present | |
| | | | | EGFRvIII | | |
| NCT01732640 | Head and neck | afatinib | EGFRvIII | FA | Present | |
| | squamous | | | EGFRvIII | | |
| | carcinoma | | | | | |
| NCT01783587 | Head and neck | afatinib | EGFRvIII | FA | Present | |
| | squamous | | | EGFRvIII | | |
| | carcinoma | | | | | |
| NCT01345682 | Head and neck | afatinib | EGFRvIII | FA | Present | |
| | squamous | | | EGFRvIII | | |
| | carcinoma | | | | | |
| NCT01721525 | Head and neck | afatinib | EGFRvIII | FA | Present | |
| | squamous | | | EGFRvIII | | |
| | carcinoma | | | | | |
| NCT01646125 | BAC, NSCLC | AUY922 | EGFR | Seq. EGFR | Activating | |
| | | | | | Mutation \| | |
| | | | | | Exon 21 | |
| | | | | | L858R \| | |
| | | | | | Exon 19 del | |
| NCT01526928 | BAC, NSCLC | CO-1686 | EGFR | Seq. EGFR | Activating | |
| | | | | | Mutation \| | |
| | | | | | Exon 21 | |
| | | | | | L858R \| | |
| | | | | | Exon 19 del | |
| NCT01620190 | BAC, NSCLC | nab-paclitaxel | EGFR | Seq. EGFR | Activating | |
| | | | | | Mutation \| | |
| | | | | | Exon 21 | |
| | | | | | L858R \| | |
| | | | | | Exon 19 del | |
| NCT01532089 | BAC, NSCLC | erlotinib | EGFR | Seq. EGFR | Activating | |
| | | | | | Mutation \| | |
| | | | | | Exon 21 | |
| | | | | | L858R \| | |
| | | | | | Exon 19 del | |
| NCT01746251 | BAC, NSCLC | afatinib | EGFR | Seq. EGFR | Activating | |
| | | | | | Mutation \| | |
| | | | | | Exon 21 | |
| | | | | | L858R \| | |
| | | | | | Exon 19 del | |
| NCT01866410 | BAC, NSCLC | cabozantinib, | EGFR | Seq. EGFR | Activating | |
| | | erlotinib | | | Mutation \| | |
| | | | | | Exon 21 | |
| | | | | | L858R \| | |
| | | | | | Exon 19 del | |
| NCT01553942 | BAC, NSCLC | afatinib | EGFR | Seq. EGFR | Activating | |
| | | | | | Mutation \| | |
| | | | | | Exon 21 | |
| | | | | | L858R \| | |
| | | | | | Exon 19 del | |
| NCT01836341 | BAC, NSCLC | afatinib | EGFR | Seq. EGFR | Activating | |
| | | | | | Mutation \| | |
| | | | | | Exon 21 | |
| | | | | | L858R \| | |
| | | | | | Exon 19 del | |
| NCT01465802 | BAC, NSCLC | dacomitinib | EGFR | Seq. EGFR | Activating | |
| | | | | | Mutation \| | |
| | | | | | Exon 21 | |
| | | | | | L858R \| | |
| | | | | | Exon 19 del | |
| NCT01324479 | All | INC280 | cMET | Seq. | Mutated | |
| | | | | CMET | | |
| NCT01324479 | All | INC280 | cMET | IHC CMET | Positive | |
| NCT01324479 | All | 1NC280 | cMET | ISH CMET | Amplified | |
| NCT00585195 | All | crizotinib | cMET | Seq. | Mutated | |
| | | | | CMET | | |
| NCT00585195 | All | crizotinib | cMET | IHC CMET | Positive | |
| NCT00585195 | All | crizotinib | cMET | ISH CMET | Amplified | |
| NCT01755767 | Liver | tivanitinib | cMET | IHC CMET | Positive | |
| | Hepatocellular | | | | | |
| | Carcinoma | | | | | |
| NCT00697632 | All | MGCD265 | cMET | Seq. | Mutated | |
| | | | | CMET | | |
| NCT00697632 | All | MGCD265 | cMET | IHC CMET | Positive | |
| NCT00697632 | All | MGCD265 | cMET | ISH CMET | Amplified | |
| NCT01654965 | All | tivanitinb | cMET | Seq. | Mutated | |
| | | | | CMET | | |
| NCT01654965 | All | tivanitinb | cMET | IHC CMET | Positive | |
| NCT01654965 | All | tivanitinb | cMET | ISH CMET | Amplified | |
| NCT01822522 | All | cabozantinib | cMET | Seq. | Mutated | |
| | | | | CMET | | |
| NCT01822522 | All | cabozantinib | cMET | IHC CMET | Positive | |
| NCT01822522 | All | cabozantinib | cMET | ISH CMET | Amplified | |
| NCT01611857 | All | tivantinib | cMET | Seq. | Mutated | |
| | | | | CMET | | |
| NCT01611857 | All | tivantinib | cMET | IHC CMET | Positive | |
| NCT01611857 | All | tivantinib | cMET | ISH CMET | Amplified | |
| NCT01625156 | All | tivantinib | cMET | Seq. | Mutated | |
| | | | | CMET | | |
| NCT01625156 | All | tivantinib | cMET | IHC CMET | Positive | |
| NCT01625156 | All | tivantinib | cMET | ISH CMET | Amplified | |
| NCT01749384 | All | tivanitinb | cMET | Seq. | Mutated | |
| | | | | CMET | | |
| NCT01749384 | All | tivanitinb | cMET | IHC CMET | Positive | |
| NCT01749384 | All | tivanitinb | cMET | ISH CMET | Amplified | |
| | NCT Number | Result | Test | Result | Test | Result |
| | NCT00326495 | | | | | |
| | NCT00326495 | | | | | |
| | NCT00574769 | | | | | |
| | NCT00574769 | | | | | |
| | NCT00574769 | | | | | |
| | NCT00585195 | | | | | |
| | NCT00585195 | | | | | |
| | NCT00610948 | | | | | |
| | NCT00610948 | | | | | |
| | NCT00622466 | | | | | |
| | NCT00625378 | | | | | |
| | NCT00625378 | | | | | |
| | NCT00756340 | | | | | |
| | NCT00756340 | | | | | |
| | NCT00770263 | | | | | |
| | NCT00770263 | | | | | |
| | NCT00780494 | | | | | |
| | NCT00912340 | No Data | ISH | No Data | | |
| | | | Her2 | | | |
| | NCT00912340 | No Data | ISH | Equiv. | | |
| | | | Her2 | Low | | |
| | NCT00912340 | No Data | ISH | Not | | |
| | | | Her2 | Amplified | | |
| | NCT00912340 | Equiv. | ISH | No Data | | |
| | | | Her2 | | | |
| | NCT00912340 | Equiv. | ISH | Equiv. | | |
| | | | Her2 | Low | | |
| | NCT00912340 | Equiv. | ISH | Not | | |
| | | | Her2 | Amplified | | |
| | NCT00912340 | Negative | ISH | No Data | | |
| | | | Her2 | | | |
| | NCT00912340 | Negative | ISH | Equiv. | | |
| | | | Her2 | Low | | |
| | NCT00912340 | Negative | ISH | Not | | |
| | | | Her2 | Amplified | | |
| | NCT00912340 | No Data | ISH | No Data | | |
| | | | Her2 | | | |
| | NCT00912340 | No Data | ISH | Equiv. | | |
| | | | Her2 | Low | | |
| | NCT00912340 | No Data | ISH | Not | | |
| | | | Her2 | Amplified | | |
| | NCT00912340 | Equiv. | ISH | No Data | | |
| | | | Her2 | | | |
| | NCT00912340 | Equiv. | ISH | Equiv. | | |
| | | | Her2 | Low | | |
| | NCT00912340 | Equiv. | ISH | Not | | |
| | | | Her2 | Amplified | | |
| | NCT00912340 | Negative | ISH | No Data | | |
| | | | Her2 | | | |
| | NCT00912340 | Negative | ISH | Not | | |
| | | | Her2 | Amplified | | |
| | NCT00912340 | Negative | ISH | Negative | | |
| | | | Her2 | | | |
| | NCT00934895 | Equiv. Low | | | | |
| | NCT00934895 | Negative | | | | |
| | NCT00934895 | Equiv. Low | | | | |
| | NCT00934895 | Negative | | | | |
| | NCT00934895 | No Data | | | | |
| | NCT00934895 | Equiv. Low | | | | |
| | NCT00934895 | Negative | | | | |
| | NCT00936858 | | | | | |
| | NCT00936858 | | | | | |
| | NCT00976573 | | | | | |
| | NCT00976573 | | | | | |
| | NCT01006369 | | | | | |
| | NCT01014936 | | | | | |
| | NCT01014936 | | | | | |
| | NCT01031381 | | | | | |
| | NCT01031381 | | | | | |
| | NCT01047293 | | | | | |
| | NCT01061788 | | | | | |
| | NCT01061788 | | | | | |
| | NCT01087554 | | | | | |
| | NCT01087554 | | | | | |
| | NCT01087983 | | | | | |
| | NCT01087983 | | | | | |
| | NCT01089101 | | | | | |
| | NCT01089101 | | | | | |
| | NCT01089101 | | | | | |
| | NCT01111825 | | | | | |
| | NCT01111825 | | | | | |
| | NCT01111825 | | | | | |
| | NCT01111825 | Negative | IHC | No Data | ISH | Equiv. |
| | | | Her2 | | Her2 | Low |
| | NCT01111825 | Negative | IHC | No Data | ISH | Negative |
| | | | Her2 | | Her2 | |
| | NCT01111825 | Negative | IHC | Equiv. | ISH | Equiv. |
| | | | Her2 | | Her2 | Low |
| | NCT01111825 | Negative | IHC | Equiv. | ISH | Negative |
| | | | Her2 | | Her2 | |
| | NCT01111825 | Negative | IHC | Negative | ISH | No Data |
| | | | Her2 | | Her2 | |
| | NCT01111825 | Negative | IHC | Negative | ISH | Equiv. |
| | | | Her2 | | Her2 | Low |
| | NCT01111825 | Negative | IHC | Negative | ISH | Negative |
| | | | Her2 | | Her2 | |
| | NCT01121575 | | | | | |
| | NCT01121575 | | | | | |
| | NCT01122199 | | | | | |
| | NCT01122199 | | | | | |
| | NCT01132664 | Positive | | | | |
| | NCT01132664 | Amplified \| | | | | |
| | | Equiv. | | | | |
| | | High | | | | |
| | NCT01132664 | Positive | | | | |
| | NCT01132664 | Amplified \| | | | | |
| | | Equiv. | | | | |
| | | High | | | | |
| | NCT01132664 | Positive | | | | |
| | NCT01132664 | Amplified \| | | | | |
| | | Equiv. | | | | |
| | | High | | | | |
| | NCT01141244 | | | | | |
| | NCT01141244 | | | | | |
| | NCT01143402 | | | | | |
| | NCT01143402 | | | | | |
| | NCT01148849 | | | | | |
| | NCT01148849 | | | | | |
| | NCT01158651 | | | | | |
| | NCT01158651 | | | | | |
| | NCT01174199 | | | | | |
| | NCT01174199 | | | | | |
| | NCT01182168 | | | | | |
| | NCT01182168 | | | | | |
| | NCT01187199 | | | | | |
| | NCT01187199 | | | | | |
| | NCT01191697 | | | | | |
| | NCT01191697 | Positive | | | | |
| | NCT01191697 | Amplified \| | | | | |
| | | Equiv. | | | | |
| | | High | | | | |
| | NCT01194869 | Negative | IHC | Negative | IHC | |
| | | | PR | | Her2 | |
| | | | | | Negative | |
| | NCT01194869 | Negative | IHC | Negative | ISH | Not |
| | | | PR | | HER2/ | Amplified \| |
| | | | | | Neu | Equiv. |
| | | | | | | Low |
| | NCT01195922 | | | | | |
| | NCT01195922 | | | | | |
| | NCT01196429 | | | | | |
| | NCT01196429 | | | | | |
| | NCT01204099 | | | | | |
| | NCT01204099 | | | | | |
| | NCT01204099 | | | | | |
| | NCT01206530 | | | | | |
| | NCT01212822 | | | | | |
| | NCT01218555 | | | | | |
| | NCT01218555 | | | | | |
| | NCT01219699 | | | | | |
| | NCT01219699 | | | | | |
| | NCT01219699 | | | | | |
| | NCT01222715 | | | | | |
| | NCT01222715 | | | | | |
| | NCT01231399 | | | | | |
| | NCT01231399 | | | | | |
| | NCT01231594 | | | | | |
| | NCT01231594 | | | | | |
| | NCT01235897 | Positive | | | | |
| | NCT01235897 | Positive | | | | |
| | NCT01235897 | Amplified \| | | | | |
| | | Equiv. | | | | |
| | | High | | | | |
| | NCT01235897 | Amplified \| | | | | |
| | | Equiv. | | | | |
| | | High | | | | |
| | NCT01235897 | Positive | | | | |
| | NCT01235897 | Amplified \| | | | | |
| | | Equiv. | | | | |
| | | High | | | | |
| | NCT01245205 | | | | | |
| | NCT01245205 | | | | | |
| | NCT01245205 | | | | | |
| | NCT01248247 | | | | | |
| | NCT01248247 | | | | | |
| | NCT01248247 | | | | | |
| | NCT01251861 | | | | | |
| | NCT01251861 | | | | | |
| | NCT01251861 | | | | | |
| | NCT01252251 | | | | | |
| | NCT01252251 | | | | | |
| | NCT01256268 | | | | | |
| | NCT01256268 | | | | | |
| | NCT01256385 | | | | | |
| | NCT01256385 | | | | | |
| | NCT01270321 | | | | | |
| | NCT01270321 | | | | | |
| | NCT01276210 | | | | | |
| | NCT01276210 | | | | | |
| | NCT01277757 | | | | | |
| | NCT01277757 | | | | | |
| | NCT01277757 | | | | | |
| | NCT01279681 | | | | | |
| | NCT01281163 | Positive | | | | |
| | NCT01281163 | Amplified \| | | | | |
| | | Equiv. | | | | |
| | | High | | | | |
| | NCT01281163 | Positive | | | | |
| | NCT01281163 | Amplified \| | | | | |
| | | Equiv. | | | | |
| | | High | | | | |
| | NCT01281163 | Positive | | | | |
| | NCT01281163 | Amplified \| | | | | |
| | | Equiv. | | | | |
| | | High | | | | |
| | NCT01281514 | | | | | |
| | NCT01281514 | | | | | |
| | NCT01283789 | | | | | |
| | NCT01283789 | | | | | |
| | NCT01283789 | | | | | |
| | NCT01297452 | | | | | |
| | NCT01297452 | | | | | |
| | NCT01297452 | | | | | |
| | NCT01297491 | | | | | |
| | NCT01297491 | | | | | |
| | NCT01297491 | | | | | |
| | NCT01298570 | | | | | |
| | NCT01298570 | | | | | |
| | NCT01298570 | | | | | |
| | NCT01300429 | | | | | |
| | NCT01300962 | | | | | |
| | NCT01300962 | | | | | |
| | NCT01300962 | | | | | |
| | NCT01301716 | | | | | |
| | NCT01301716 | | | | | |
| | NCT01301716 | | | | | |
| | NCT01304602 | | | | | |
| | NCT01304602 | | | | | |
| | NCT01304602 | | | | | |
| | NCT01305941 | | | | | |
| | NCT01305941 | | | | | |
| | NCT01305941 | | | | | |
| | NCT01306045 | | | | | |
| | NCT01306045 | | | | | |
| | NCT01306045 | | | | | |
| | NCT01307631 | | | | | |
| | NCT01307631 | | | | | |
| | NCT01307631 | | | | | |
| | NCT01313039 | Negative | | | | |
| | NCT01313039 | Negative | | | | |
| | NCT01313039 | Negative | | | | |
| | NCT01319539 | | | | | |
| | NCT01319539 | | | | | |
| | NCT01319539 | | | | | |
| | NCT01320085 | | | | | |
| | NCT01320085 | | | | | |
| | NCT01320085 | | | | | |
| | NCT01322815 | | | | | |
| | NCT01331135 | | | | | |
| | NCT01331135 | | | | | |
| | NCT01333475 | | | | | |
| | NCT01333475 | | | | | |
| | NCT01333475 | | | | | |
| | NCT01339052 | | | | | |
| | NCT01339052 | | | | | |
| | NCT01339052 | | | | | |
| | NCT01339442 | Positive | | | | |
| | NCT01339442 | Positive | | | | |
| | NCT01339442 | Positive | | | | |
| | NCT01344031 | Positive | | | | |
| | NCT01344031 | Positive | | | | |
| | NCT01344031 | Positive | | | | |
| | NCT01347866 | | | | | |
| | NCT01347866 | | | | | |
| | NCT01347866 | | | | | |
| | NCT01349660 | | | | | |
| | NCT01349660 | | | | | |
| | NCT01349660 | | | | | |
| | NCT01349933 | | | | | |
| | NCT01349933 | | | | | |
| | NCT01349933 | | | | | |
| | NCT01362374 | | | | | |
| | NCT01362374 | | | | | |
| | NCT01362374 | | | | | |
| | NCT01363232 | | | | | |
| | NCT01363232 | | | | | |
| | NCT01363232 | | | | | |
| | NCT01364051 | | | | | |
| | NCT01364051 | | | | | |
| | NCT01364051 | | | | | |
| | NCT01374425 | | | | | |
| | NCT01375829 | | | | | |
| | NCT01375829 | | | | | |
| | NCT01376310 | | | | | |
| | NCT01376310 | | | | | |
| | NCT01376310 | | | | | |
| | NCT01376453 | | | | | |
| | NCT01376453 | | | | | |
| | NCT01385228 | | | | | |
| | NCT01385293 | | | | | |
| | NCT01385293 | | | | | |
| | NCT01385293 | | | | | |
| | NCT01386450 | | | | | |
| | NCT01386450 | | | | | |
| | NCT01386450 | | | | | |
| | NCT01390818 | | | | | |
| | NCT01390818 | | | | | |
| | NCT01392521 | | | | | |
| | NCT01392521 | | | | | |
| | NCT01392521 | | | | | |
| | NCT01396148 | | | | | |
| | NCT01409200 | | | | | |
| | NCT01420081 | | | | | |
| | NCT01420081 | | | | | |
| | NCT01420081 | | | | | |
| | NCT01427946 | | | | | |
| | NCT01434602 | | | | | |
| | NCT01434602 | | | | | |
| | NCT01437566 | | | | | |
| | NCT01437566 | | | | | |
| | NCT01437566 | | | | | |
| | NCT01441947 | | | | | |
| | NCT01449058 | | | | | |
| | NCT01449058 | | | | | |
| | NCT01449058 | | | | | |
| | NCT01449058 | | | | | |
| | NCT01449058 | | | | | |
| | NCT01449058 | | | | | |
| | NCT01450384 | | | | | |
| | NCT01450384 | | | | | |
| | NCT01465802 | | | | | |
| | NCT01465802 | | | | | |
| | NCT01466244 | | | | | |
| | NCT01466244 | | | | | |
| | NCT01466972 | | | | | |
| | NCT01468688 | | | | | |
| | NCT01468688 | | | | | |
| | NCT01468688 | | | | | |
| | NCT01469572 | | | | | |
| | NCT01469572 | | | | | |
| | NCT01470209 | | | | | |
| | NCT01470209 | | | | | |
| | NCT01470209 | | | | | |
| | NCT01471353 | | | | | |
| | NCT01473901 | | | | | |
| | NCT01473901 | | | | | |
| | NCT01473901 | | | | | |
| | NCT01480154 | | | | | |
| | NCT01480154 | | | | | |
| | NCT01480154 | | | | | |
| | NCT01482156 | | | | | |
| | NCT01482156 | | | | | |
| | NCT01482156 | | | | | |
| | NCT01488487 | | | | | |
| | NCT01488487 | | | | | |
| | NCT01490749 | | | | | |
| | NCT01490749 | | | | | |
| | NCT01490866 | | | | | |
| | NCT01495247 | Negative | | | | |
| | NCT01495247 | Not | | | | |
| | | Amplified | | | | |
| | NCT01495247 | Negative | | | | |
| | NCT01495247 | Not | | | | |
| | | Amplified | | | | |
| | NCT01495247 | Negative | | | | |
| | NCT01495247 | Not | | | | |
| | | Amplified | | | | |
| | NCT01499160 | | | | | |
| | NCT01499160 | | | | | |
| | NCT01508104 | | | | | |
| | NCT01508104 | | | | | |
| | NCT01508104 | | | | | |
| | NCT01512251 | | | | | |
| | NCT01512251 | | | | | |
| | NCT01512251 | | | | | |
| | NCT01516216 | | | | | |
| | NCT01519414 | | | | | |
| | NCT01519414 | | | | | |
| | NCT01522768 | | | | | |
| | NCT01522768 | | | | | |
| | NCT01524783 | | | | | |
| | NCT01524783 | | | | | |
| | NCT01529593 | | | | | |
| | NCT01529593 | | | | | |
| | NCT01531361 | | | | | |
| | NCT01531361 | | | | | |
| | NCT01536054 | | | | | |
| | NCT01536054 | | | | | |
| | NCT01538680 | | | | | |
| | NCT01538680 | | | | | |
| | NCT01540253 | | | | | |
| | NCT01540253 | | | | | |
| | NCT01540253 | | | | | |
| | NCT01542996 | Negative | IHC | Negative | IHC | Negative |
| | | | PR | | HER2/ | |
| | | | | | Neu | |
| | NCT01542996 | Negative | IHC | Negative | IHC | Negative |
| | | | PR | | HER2/ | |
| | | | | | Neu | |
| | NCT01542996 | Negative | IHC | Negative | ISH | Negative |
| | | | PR | | HER2/ | |
| | | | | | Neu | |
| | NCT01542996 | Negative | IHC | Negative | ISH | Negative |
| | | | PR | | HER2/ | |
| | | | | | Neu | |
| | NCT01543698 | | | | | |
| | NCT01543698 | | | | | |
| | NCT01543698 | | | | | |
| | NCT01545947 | | | | | |
| | NCT01545947 | | | | | |
| | NCT01548144 | | | | | |
| | NCT01548144 | | | | | |
| | NCT01548807 | | | | | |
| | NCT01548807 | | | | | |
| | NCT01550380 | | | | | |
| | NCT01550380 | | | | | |
| | NCT01550380 | | | | | |
| | NCT01552434 | | | | | |
| | NCT01552434 | | | | | |
| | NCT01553851 | | | | | |
| | NCT01553851 | | | | | |
| | NCT01553851 | | | | | |
| | NCT01553942 | | | | | |
| | NCT01562275 | | | | | |
| | NCT01562275 | | | | | |
| | NCT01562275 | | | | | |
| | NCT01562899 | | | | | |
| | NCT01562899 | | | | | |
| | NCT01562899 | | | | | |
| | NCT01563354 | | | | | |
| | NCT01563354 | | | | | |
| | NCT01567930 | | | | | |
| | NCT01567930 | | | | | |
| | NCT01571024 | | | | | |
| | NCT01571024 | | | | | |
| | NCT01571024 | | | | | |
| | NCT01571284 | | | | | |
| | NCT01576406 | | | | | |
| | NCT01576666 | | | | | |
| | NCT01576666 | | | | | |
| | NCT01576666 | | | | | |
| | NCT01579994 | | | | | |
| | NCT01582191 | | | | | |
| | NCT01582191 | | | | | |
| | NCT01587040 | | | | | |
| | NCT01587040 | | | | | |
| | NCT01587040 | | | | | |
| | NCT01587352 | | | | | |
| | NCT01587352 | | | | | |
| | NCT01596270 | | | | | |
| | NCT01596270 | | | | | |
| | NCT01596270 | | | | | |
| | NCT01661972 | | | | | |
| | NCT01693068 | | | | | |
| | NCT01745367 | Negative | IHC | Negative | IHC | |
| | | | PR | | Her2 | |
| | | | | | Negative | |
| | NCT01745367 | Negative | IHC | Negative | ISH | Not |
| | | | PR | | HER2/ | Amplified \| |
| | | | | | Neu | Equiv. Low |
| | NCT01456325 | Mutated | | | | |
| | NCT01662869 | Negative | ISH | Negative | | |
| | | | HER2 | | | |
| | NCT01121575 | | | | | |
| | NCT01548144 | | | | | |
| | NCT01744652 | | | | | |
| | NCT01276041 | | | | | |
| | NCT01276041 | | | | | |
| | NCT01774786 | | | | | |
| | NCT01774786 | | | | | |
| | NCT01565083 | | | | | |
| | NCT01565083 | | | | | |
| | NCT01491737 | Positive | | | | |
| | NCT01491737 | Positive | | | | |
| | NCT01491737 | Positive | | | | |
| | NCT01491737 | Positive | | | | |
| | NCT01042379 | | | | | |
| | NCT01042379 | | | | | |
| | NCT01641939 | | | | | |
| | NCT01641939 | | | | | |
| | NCT01912963 | | | | | |
| | NCT01912963 | | | | | |
| | NCT01796197 | | | | | |
| | NCT01796197 | | | | | |
| | NCT01855828 | | | | | |
| | NCT01855828 | | | | | |
| | NCT01904903 | | | | | |
| | NCT01904903 | | | | | |
| | NCT01480479 | | | | | |
| | NCT01498328 | | | | | |
| | NCT01800695 | | | | | |
| | NCT01475006 | | | | | |
| | NCT01257594 | | | | | |
| | NCT00301418 | | | | | |
| | NCT01465802 | | | | | |
| | NCT01858389 | | | | | |
| | NCT01454596 | | | | | |
| | NCT01112527 | | | | | |
| | NCT01732640 | | | | | |
| | NCT01783587 | | | | | |
| | NCT01345682 | | | | | |
| | NCT01721525 | | | | | |
| | NCT01646125 | | | | | |
| | NCT01526928 | | | | | |
| | NCT01620190 | | | | | |
| | NCT01532089 | | | | | |
| | NCT01746251 | | | | | |
| | NCT01866410 | | | | | |
| | NCT01553942 | | | | | |
| | NCT01836341 | | | | | |
| | NCT01465802 | | | | | |
| | NCT01324479 | | | | | |
| | NCT01324479 | | | | | |
| | NCT01324479 | | | | | |
| | NCT00585195 | | | | | |
| | NCT00585195 | | | | | |
| | NCT00585195 | | | | | |
| | NCT01755767 | | | | | |
| | NCT00697632 | | | | | |
| | NCT00697632 | | | | | |
| | NCT00697632 | | | | | |
| | NCT01654965 | | | | | |
| | NCT01654965 | | | | | |
| | NCT01654965 | | | | | |
| | NCT01822522 | | | | | |
| | NCT01822522 | | | | | |
| | NCT01822522 | | | | | |
| | NCT01611857 | | | | | |
| | NCT01611857 | | | | | |
| | NCT01611857 | | | | | |
| | NCT01625156 | | | | | |
| | NCT01625156 | | | | | |
| | NCT01625156 | | | | | |
| | NCT01749384 | | | | | |
| | NCT01749384 | | | | | |
| | NCT01749384 | | | | | |

### Report

In an embodiment, the methods of the invention comprise generating a molecular profile report. The report can be delivered to the treating physician or other caregiver of the subject whose cancer has been profiled. The report can comprise multiple sections of relevant information, including without limitation: 1) a list of the genes and/or gene products in the molecular profile; 2) a description of the molecular profile of the genes and/or gene products as determined for the subject; 3) a treatment associated with one or more of the genes and/or gene products in the molecular profile; and 4) and an indication whether each treatment is likely to benefit the patient, not benefit the patient, or has indeterminate benefit. The list of the genes and/or gene products in the molecular profile can be those presented herein for the molecular intelligence profiles of the invention. The description of the molecular profile of the genes and/or gene products as determined for the subject may include such information as the laboratory technique used to assess each biomarker (e.g., RT-PCR, FISH/CISH, IHC, PCR, FA/RFLP, sequencing, etc) as well as the result and criteria used to score each technique. By way of example, the criteria for scoring a protein as positive or negative for IHC may comprise the amount of staining and/or percentage of positive cells, the criteria for scoring a nucleic acid RT-PCR may be a cycle number indicating whether the level of the appropriate nucleic acid is differentially regulated as compared to a control sample, or criteria for scoring a mutation may be a presence or absence. The treatment associated with one or more of the genes and/or gene products in the molecular profile can be determined using a rule set as described herein, e.g., in any of Tables 7-23. The indication whether each treatment is likely to benefit the patient, not benefit the patient, or has indeterminate benefit may be weighted. For example, a potential benefit may be a strong potential benefit or a lesser potential benefit. Such weighting can be based on any appropriate criteria, e.g., the strength of the evidence of the biomarker-treatment association, or the results of the profiling, e.g., a degree of over- or underexpression.

Various additional components can be added to the report as desired. In an embodiment, the report comprises a list having an indication of whether one or more of the genes and/or gene products in the molecular profile are associated with an ongoing clinical trial. The report may include identifiers for any such trials, e.g., to facilitate the treating physician's investigation of potential enrollment of the subject in the trial. In some embodiments, the report provides a list of evidence supporting the association of the genes and/or gene products in the molecular profile with the reported treatment. The list can contain citations to the evidentiary literature and/or an indication of the strength of the evidence for the particular biomarker-treatment association. In still another embodiment, the report comprises a description of the genes and/or gene products in the molecular profile. The description of the genes and/or gene products in the molecular profile may comprise without limitation the biological function and/or various treatment associations.

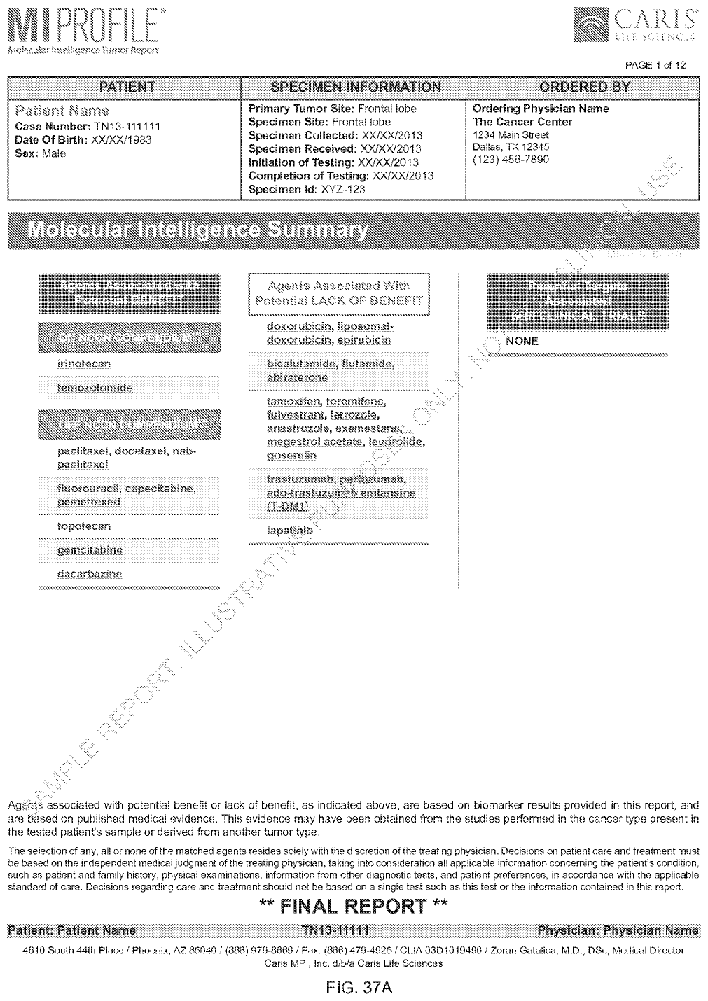

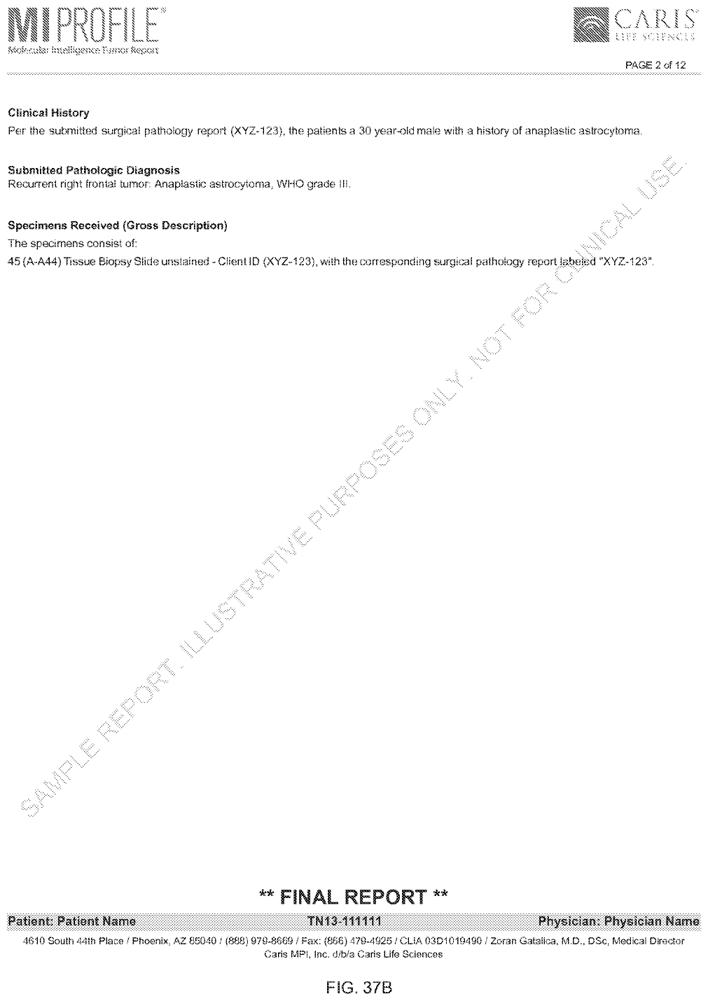

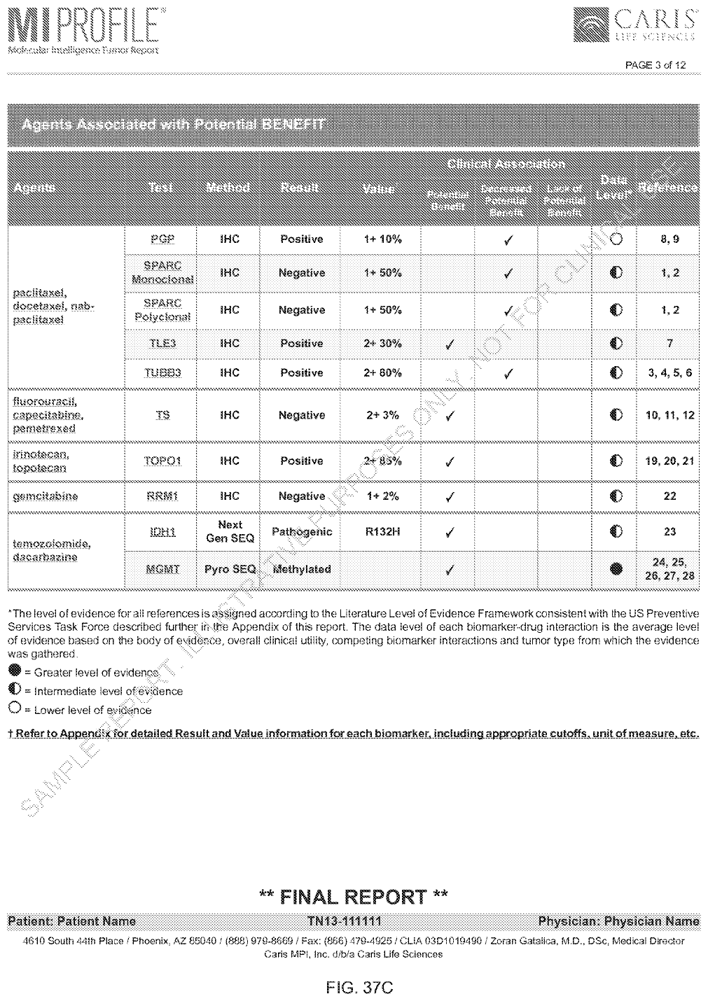

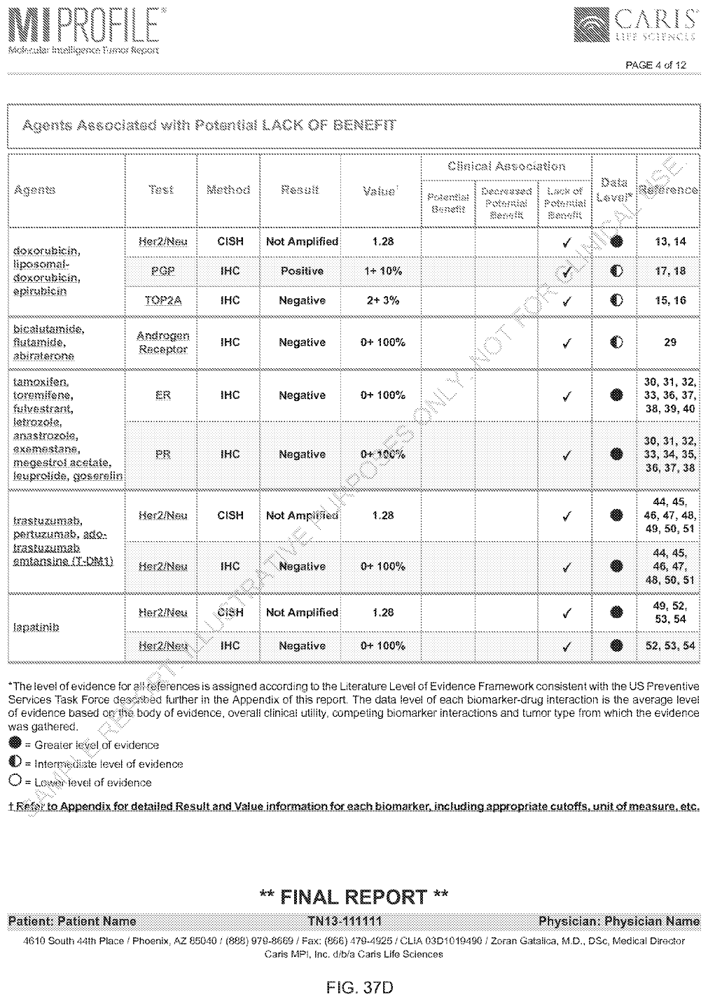

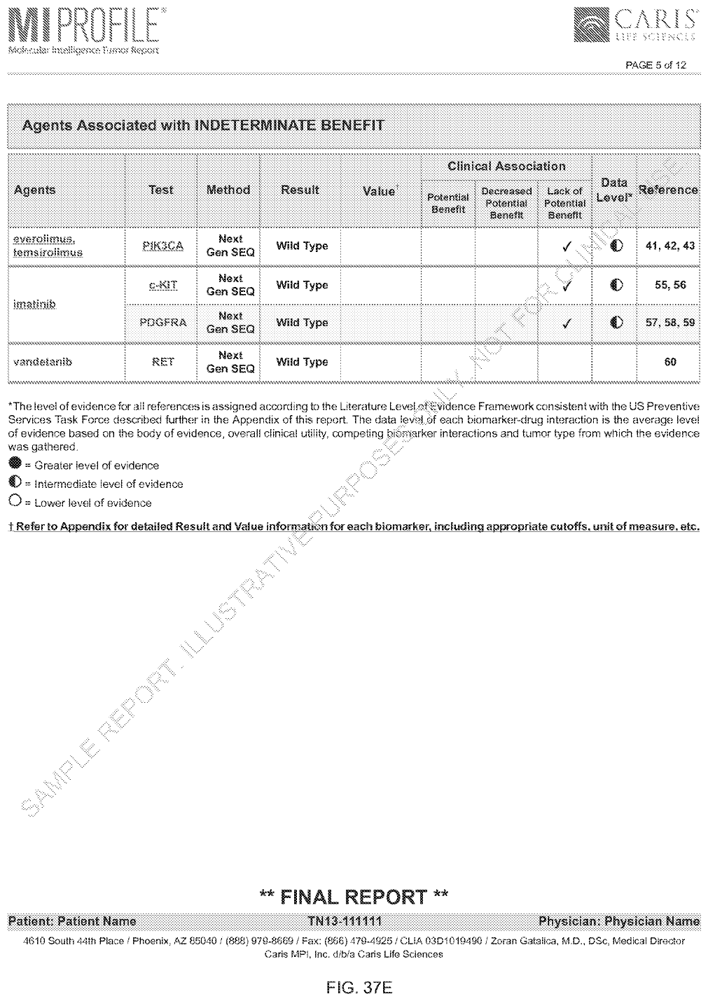

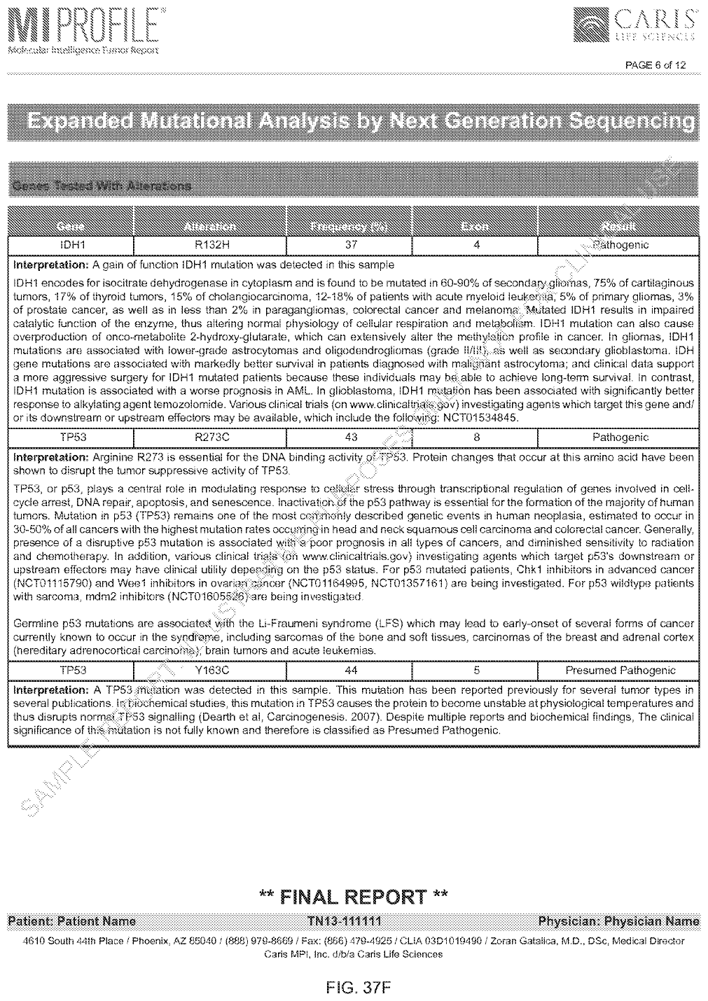

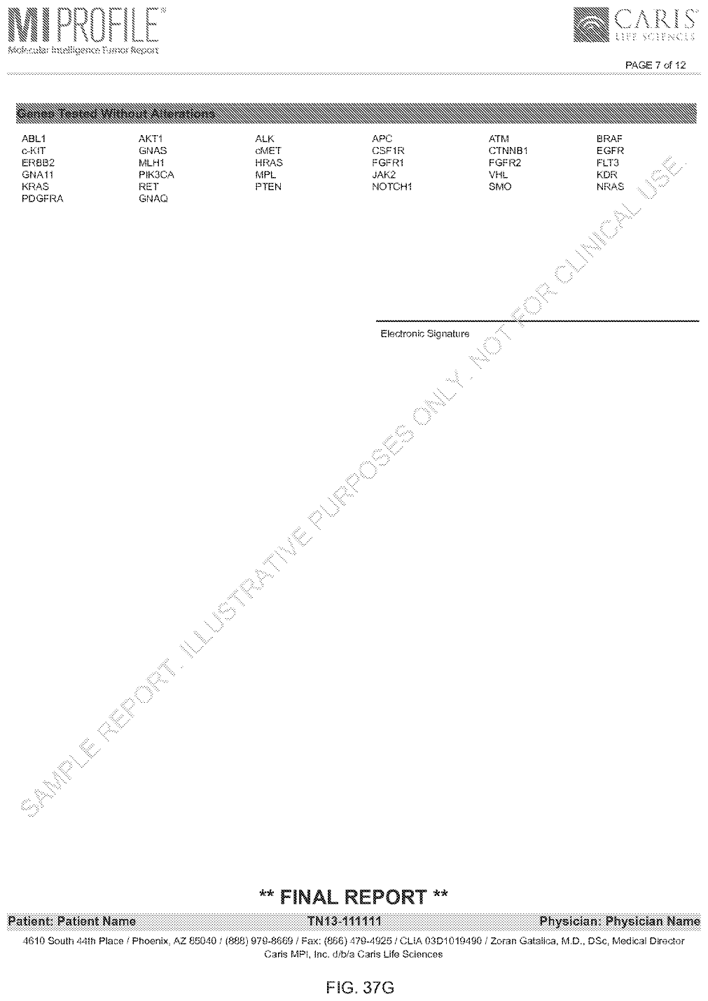

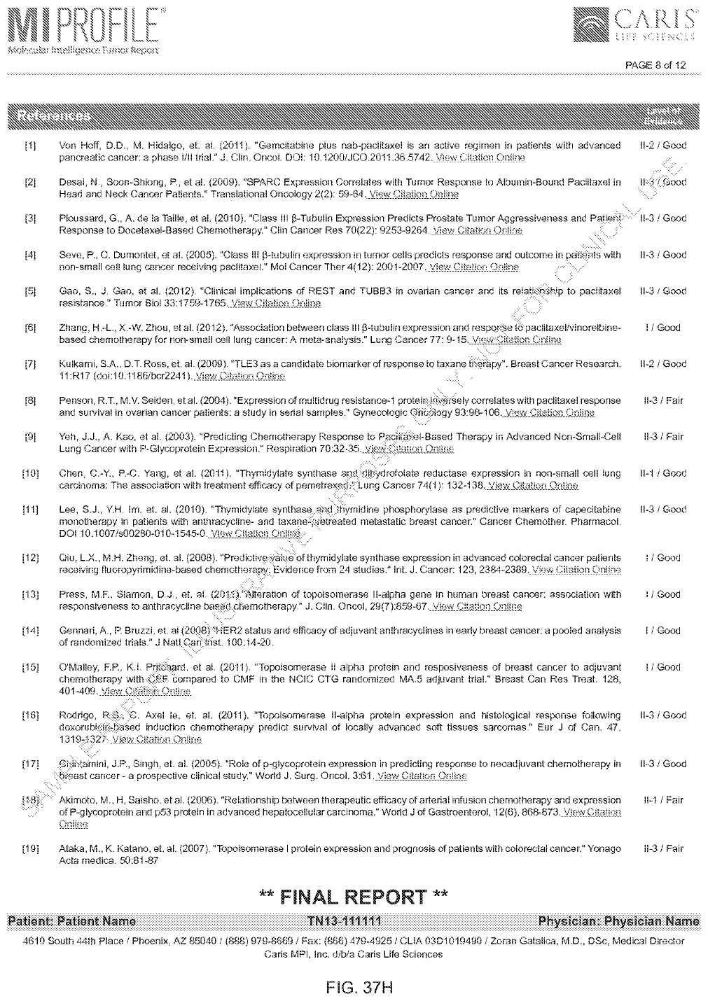

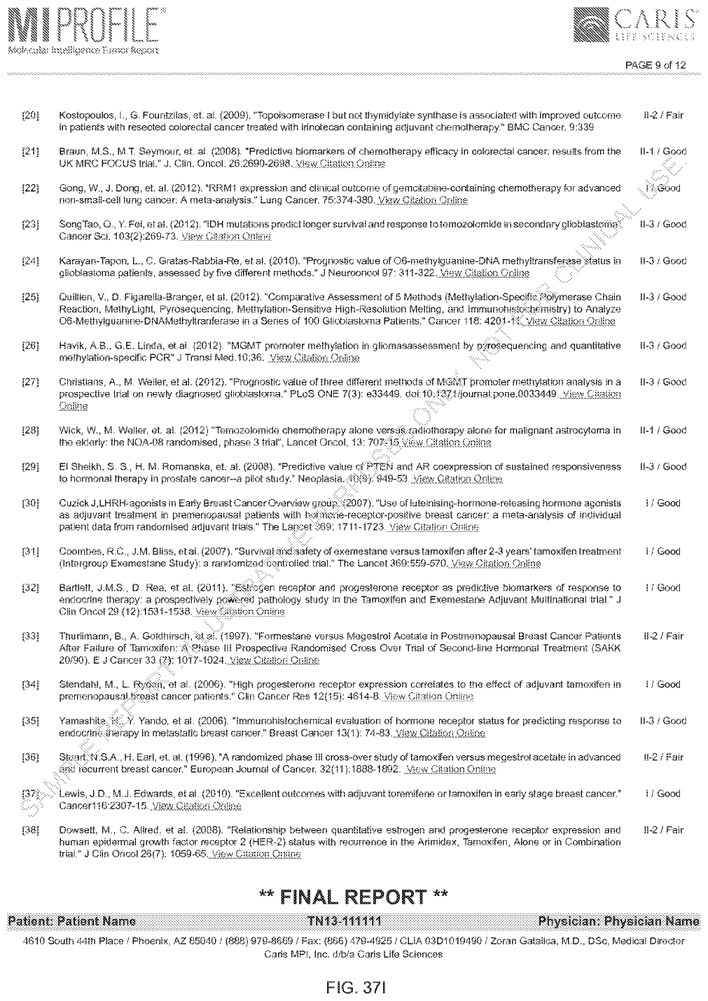

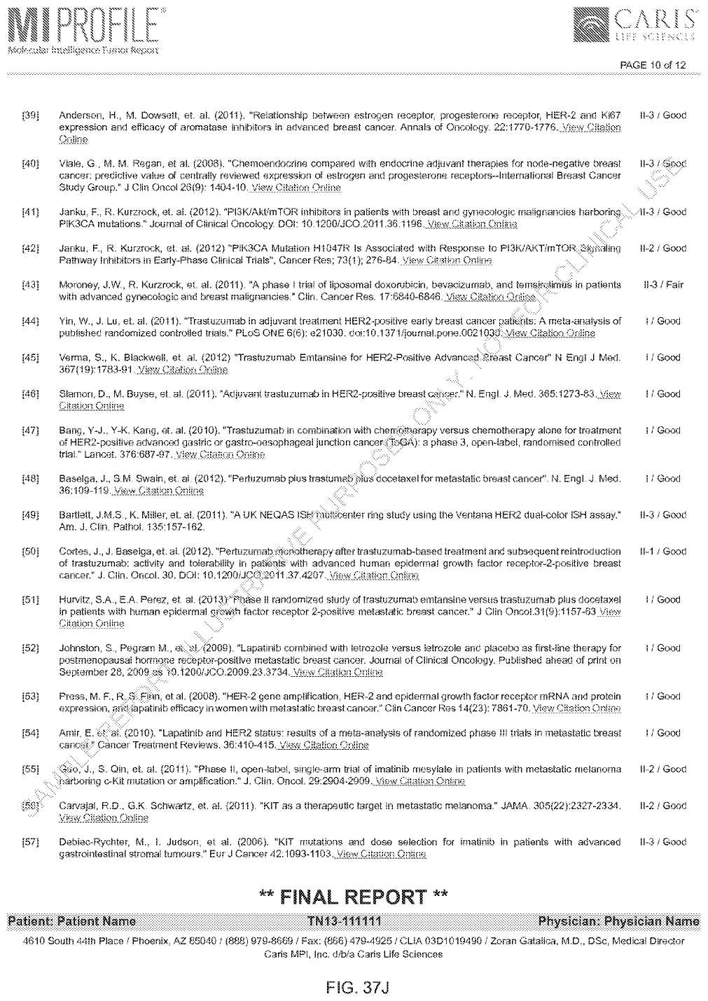

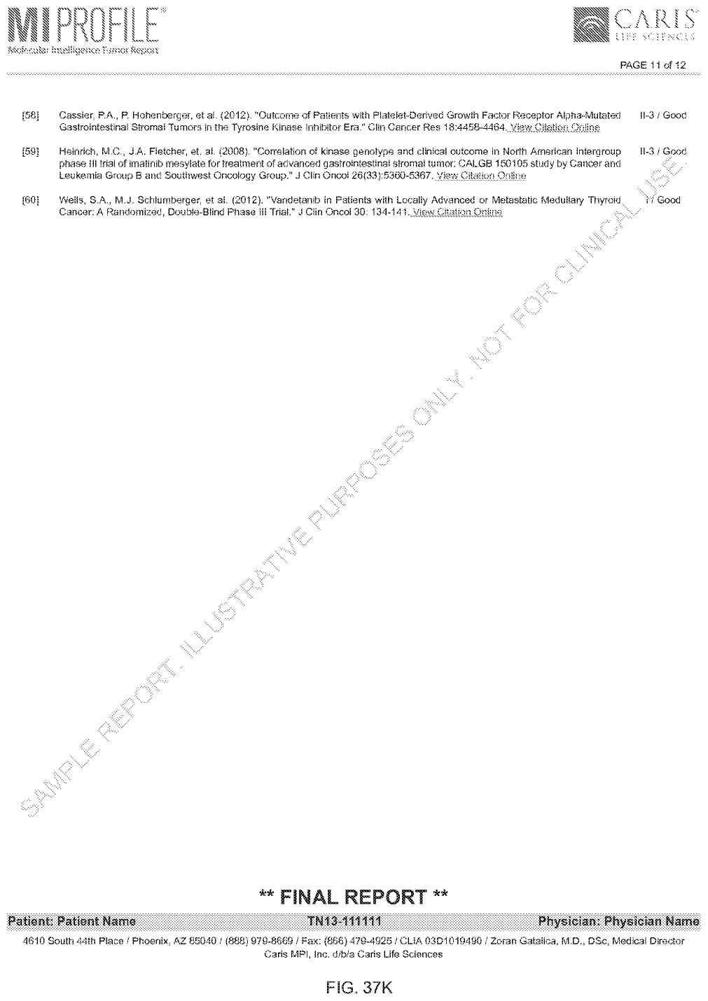

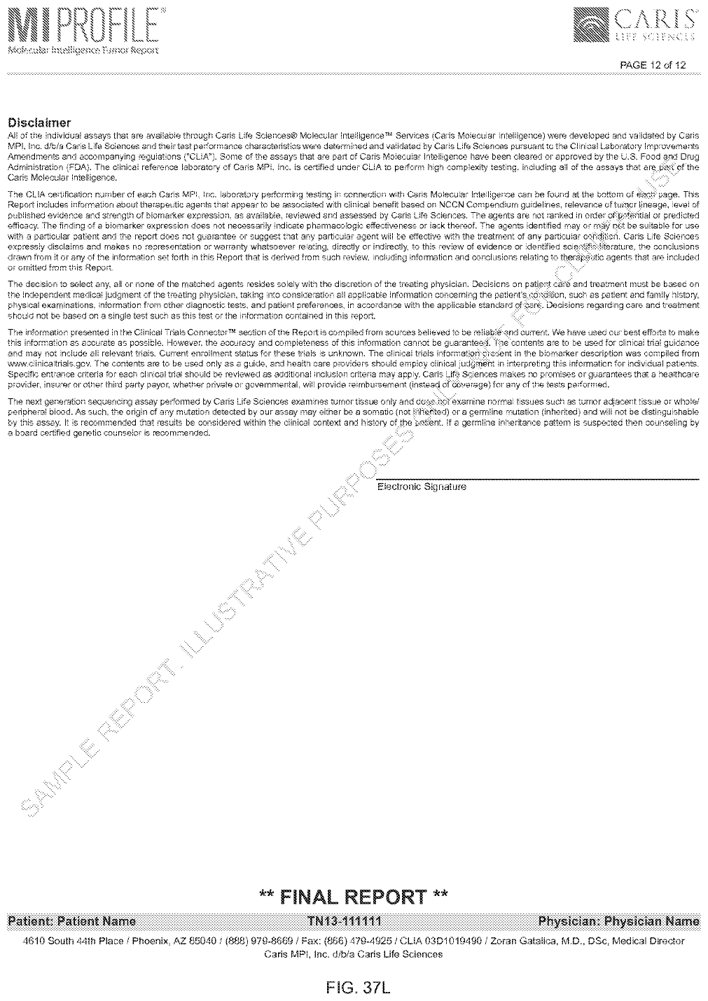

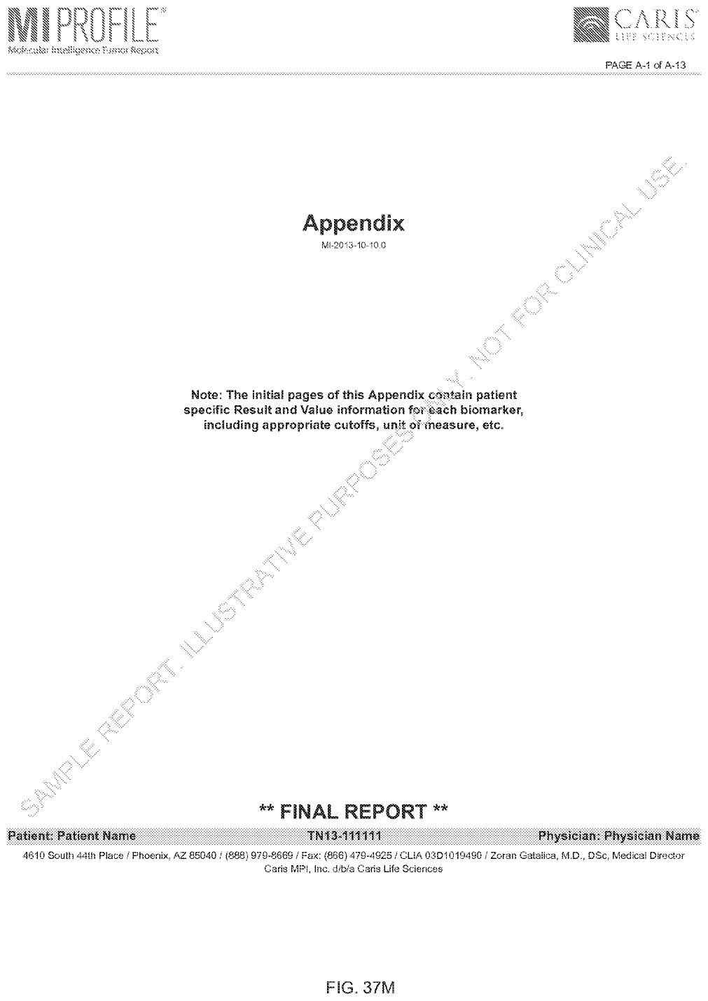

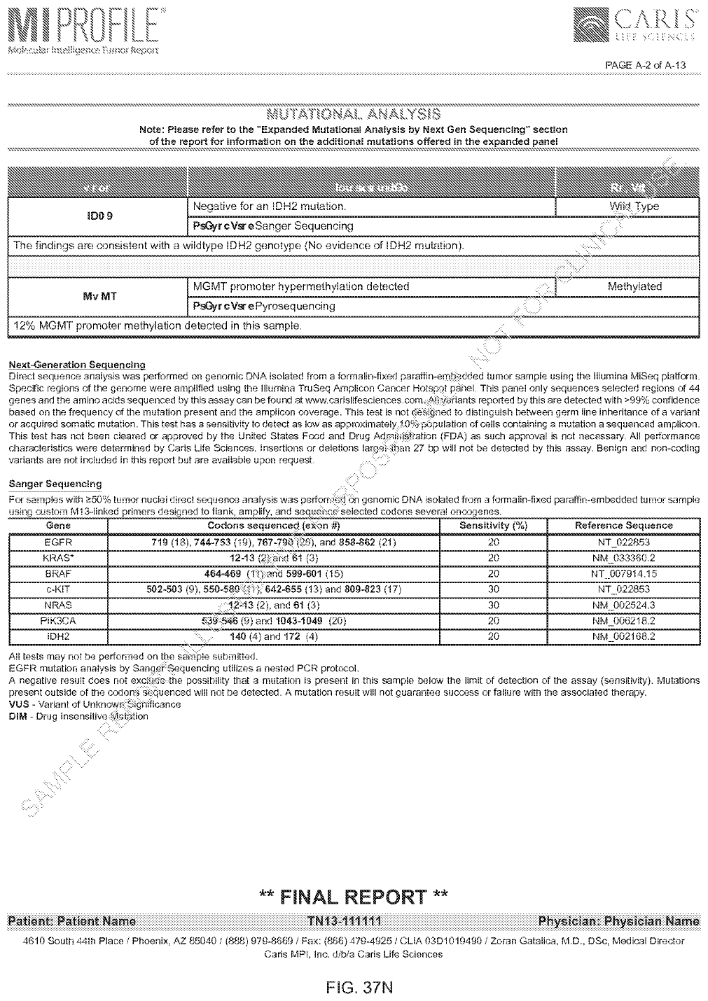

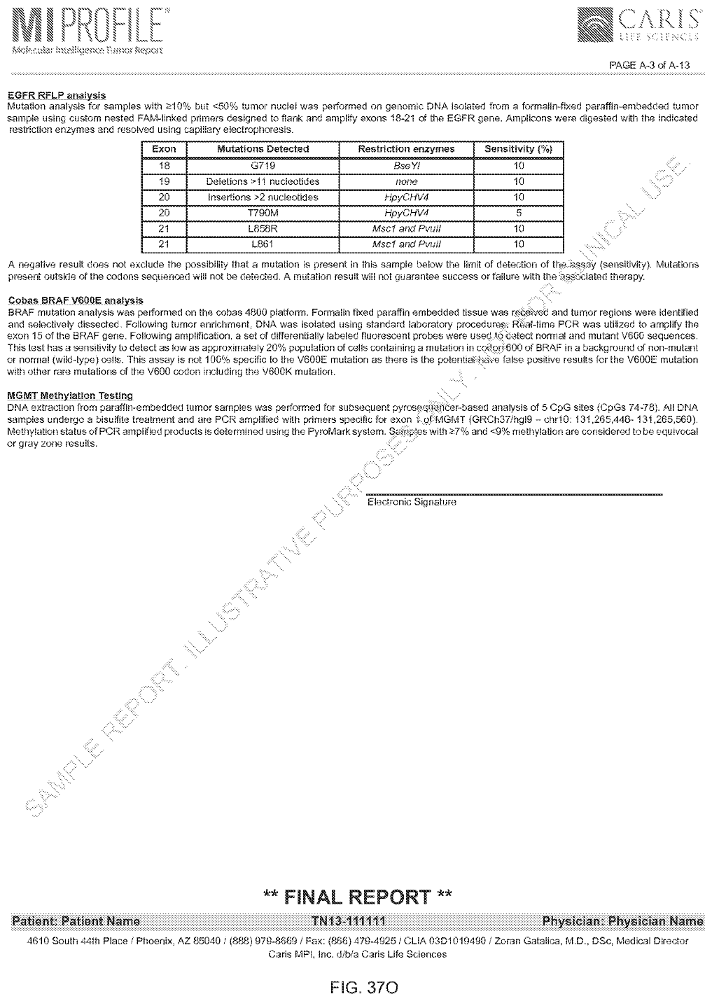

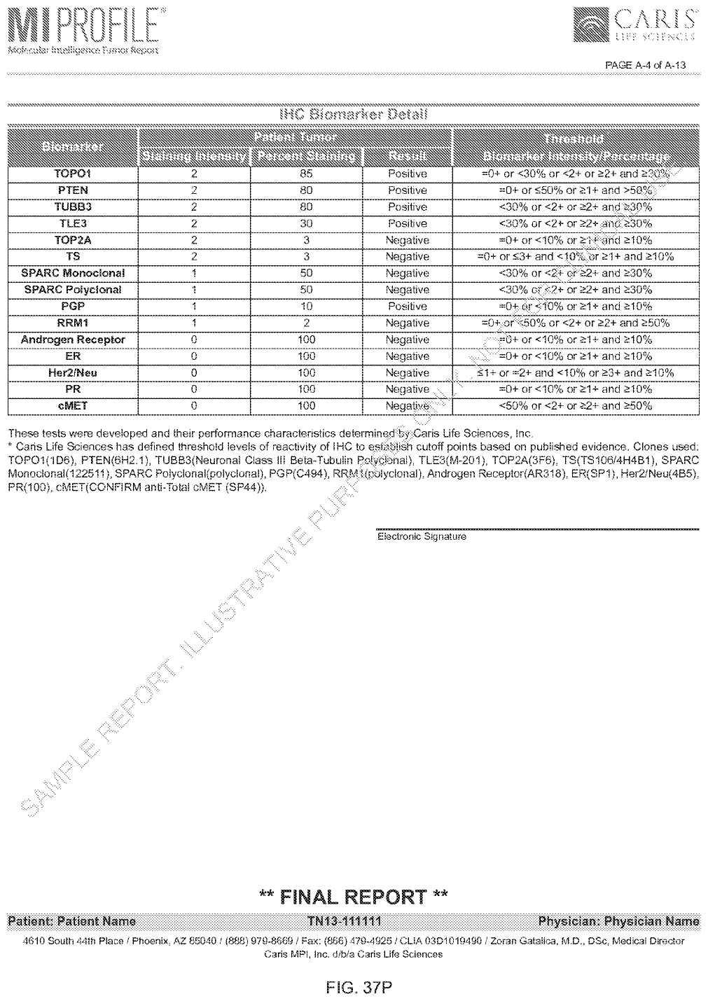

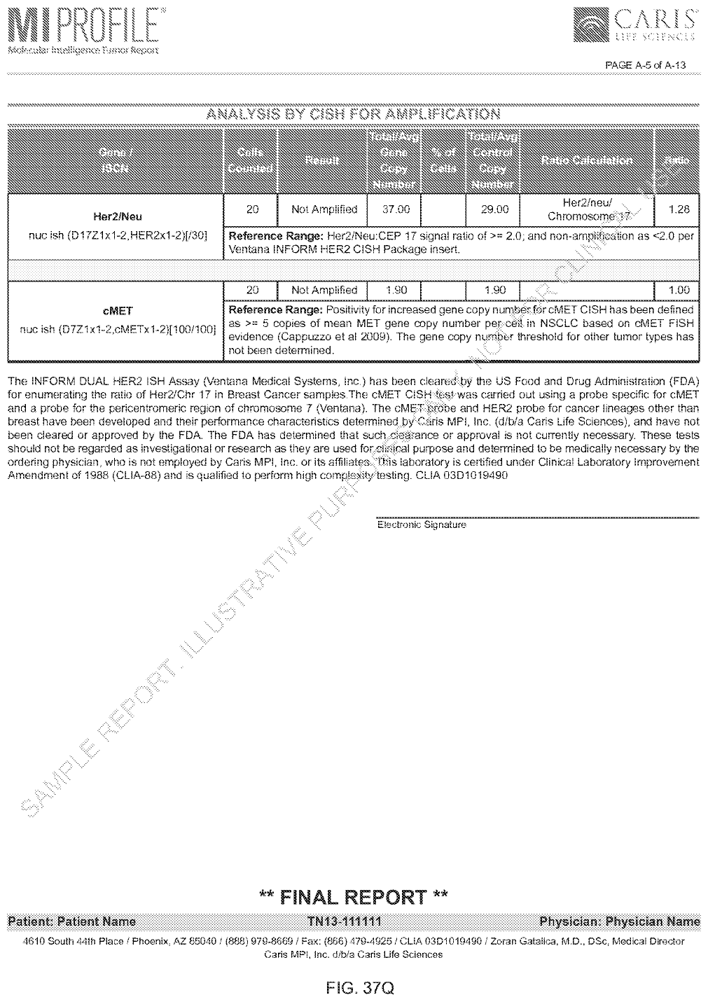

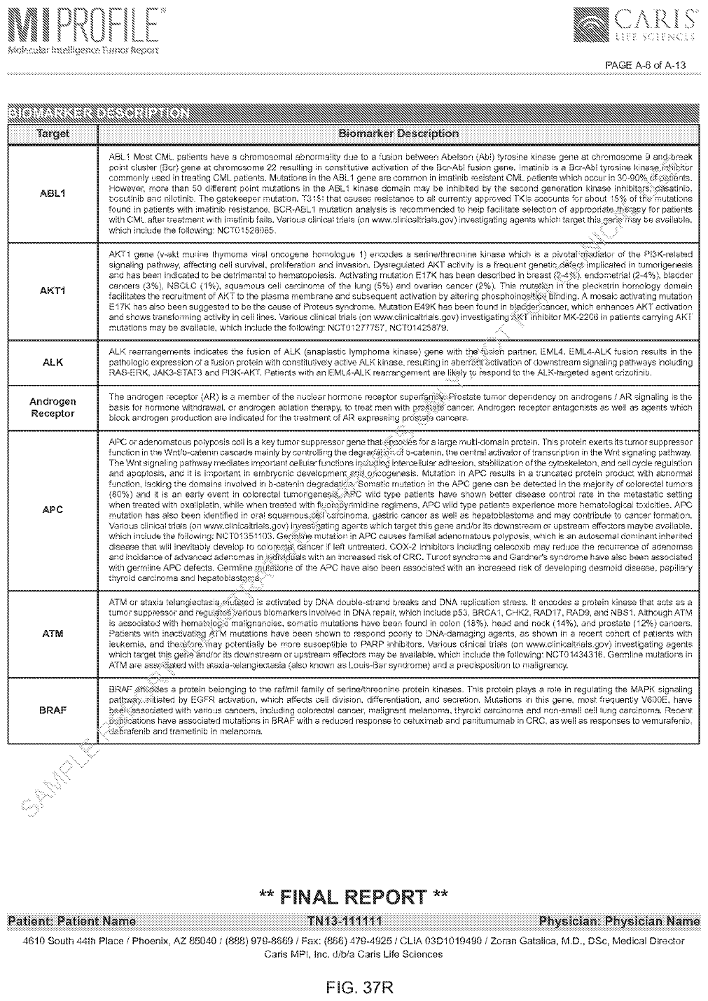

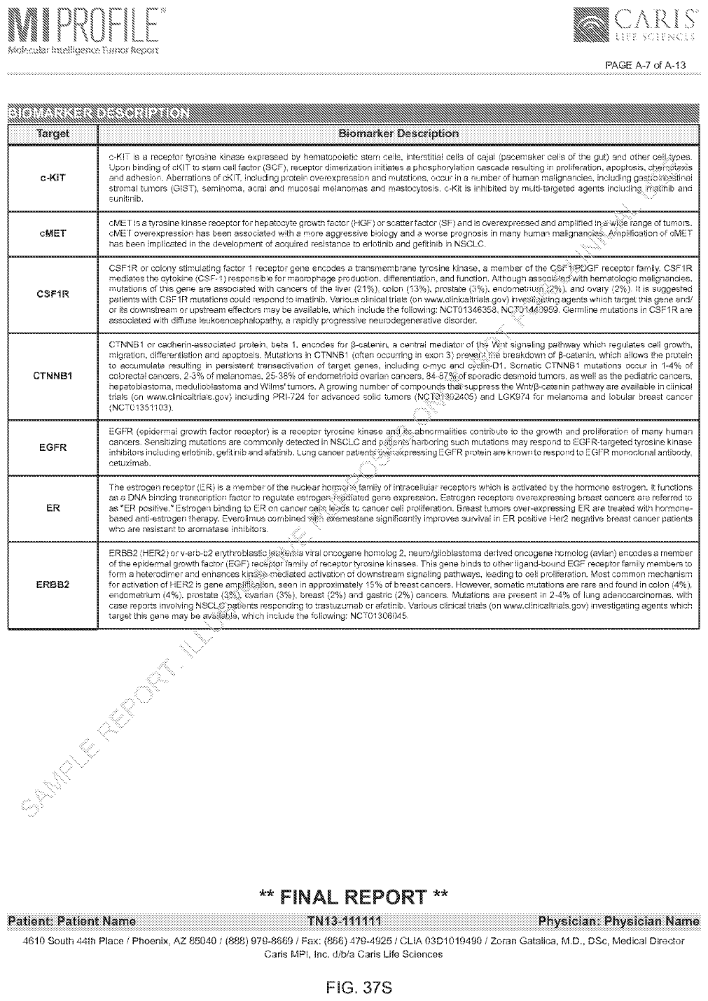

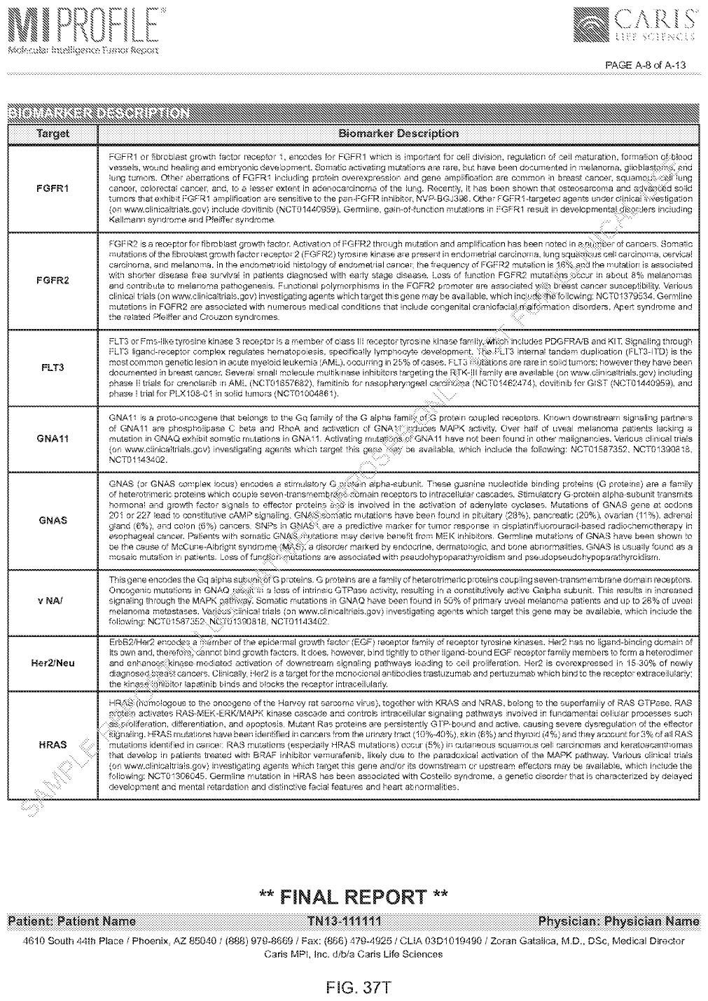

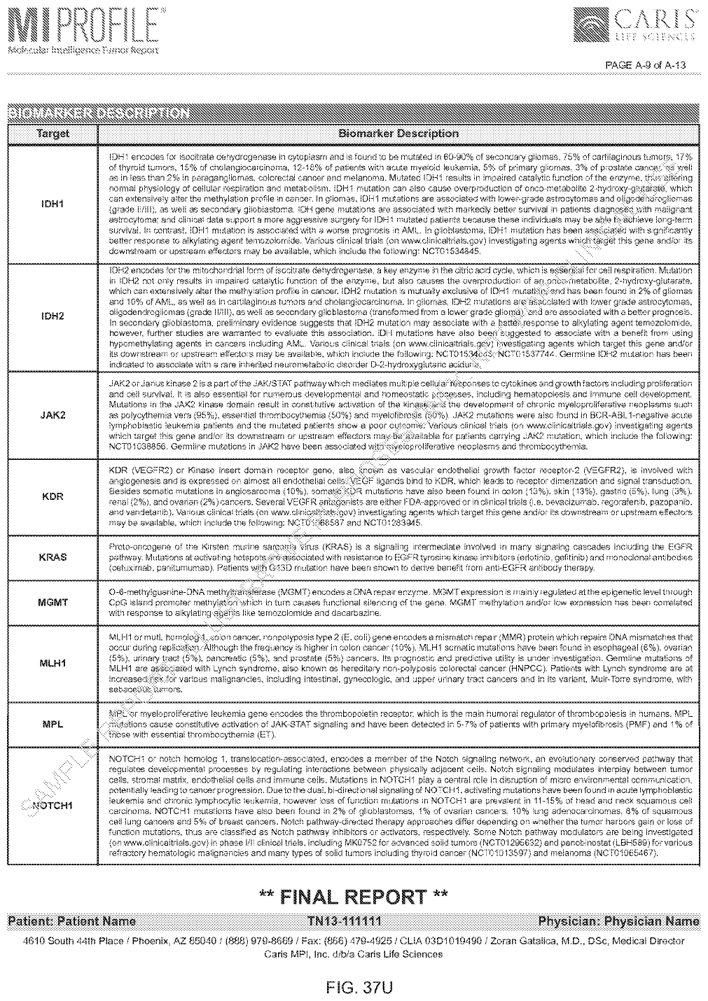

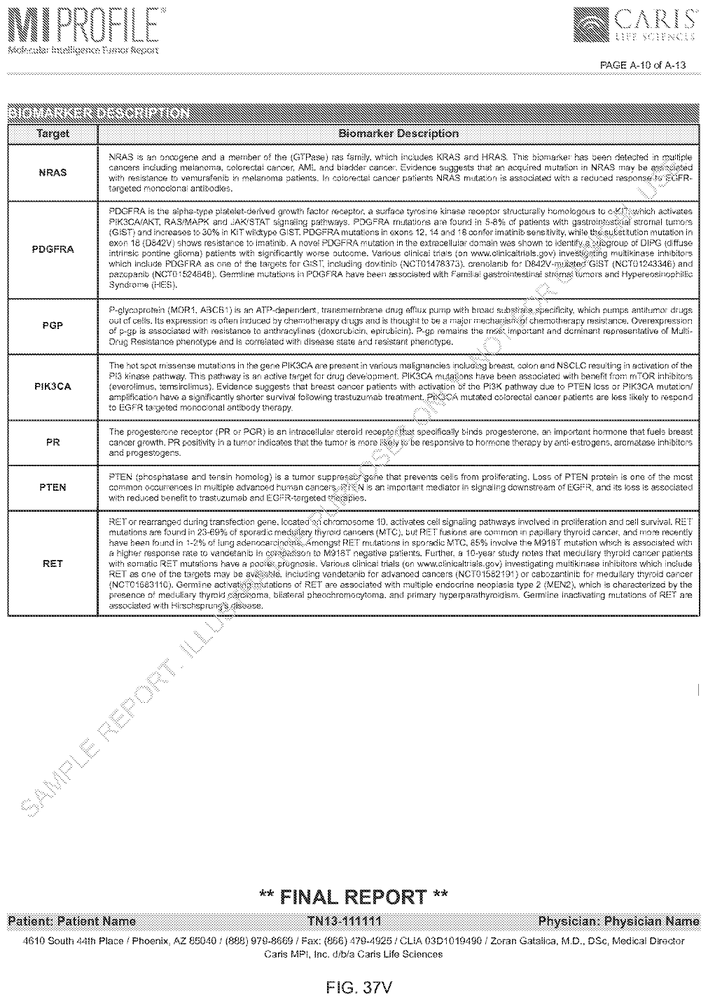

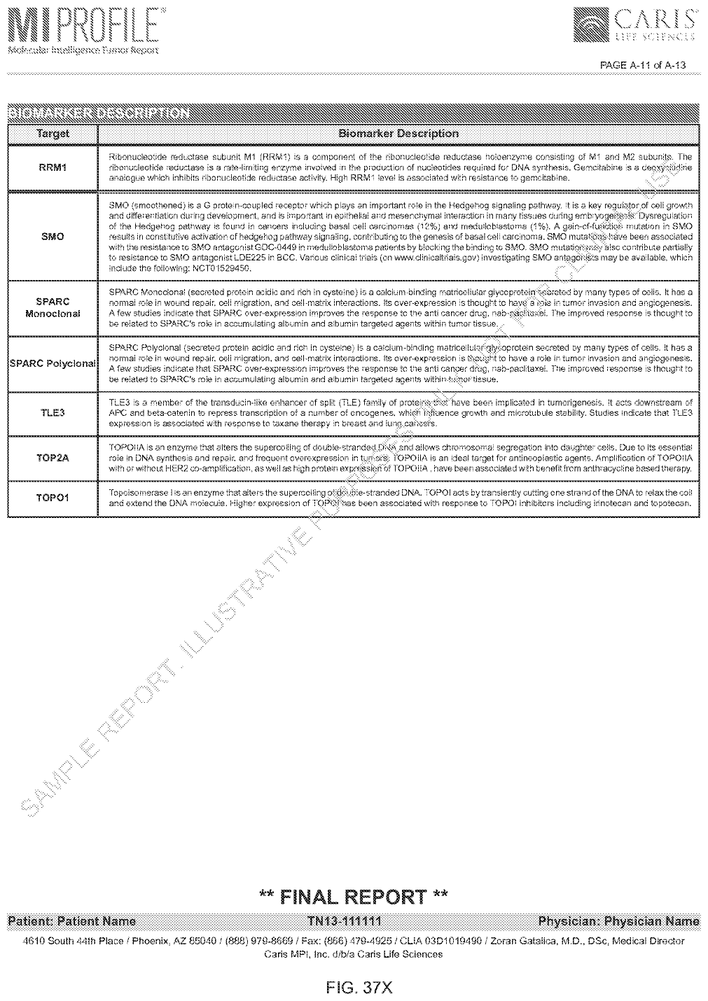

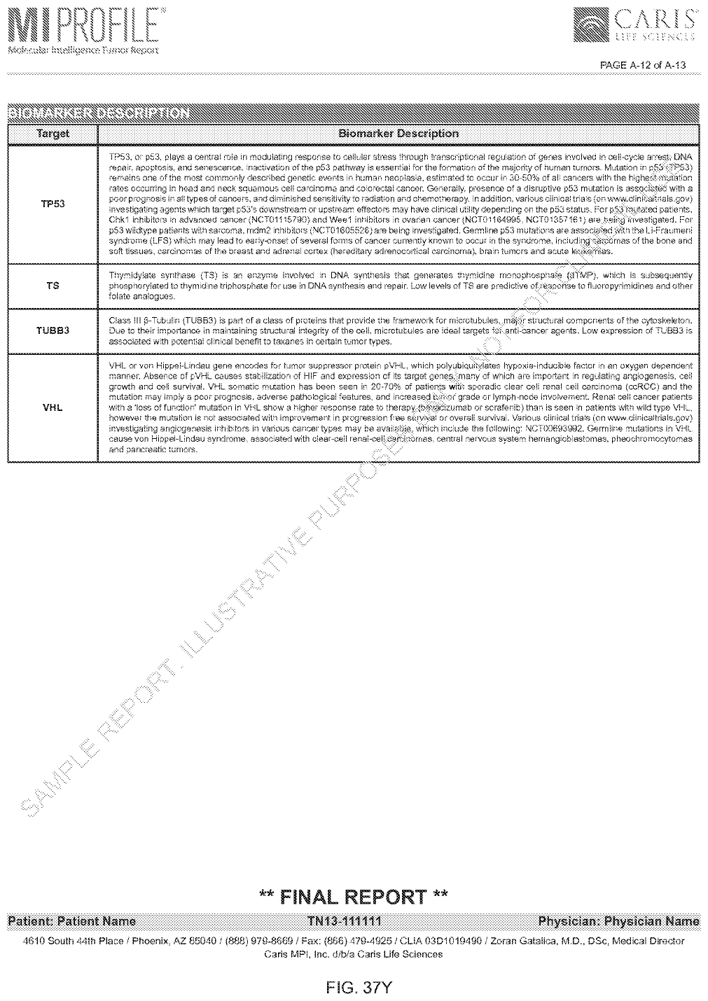

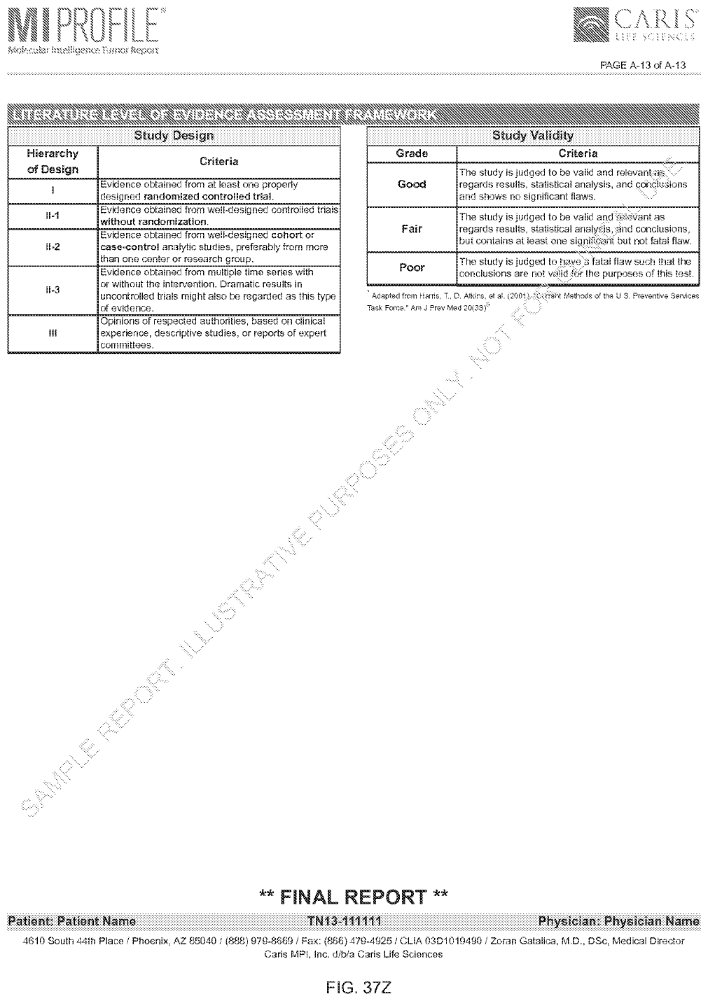

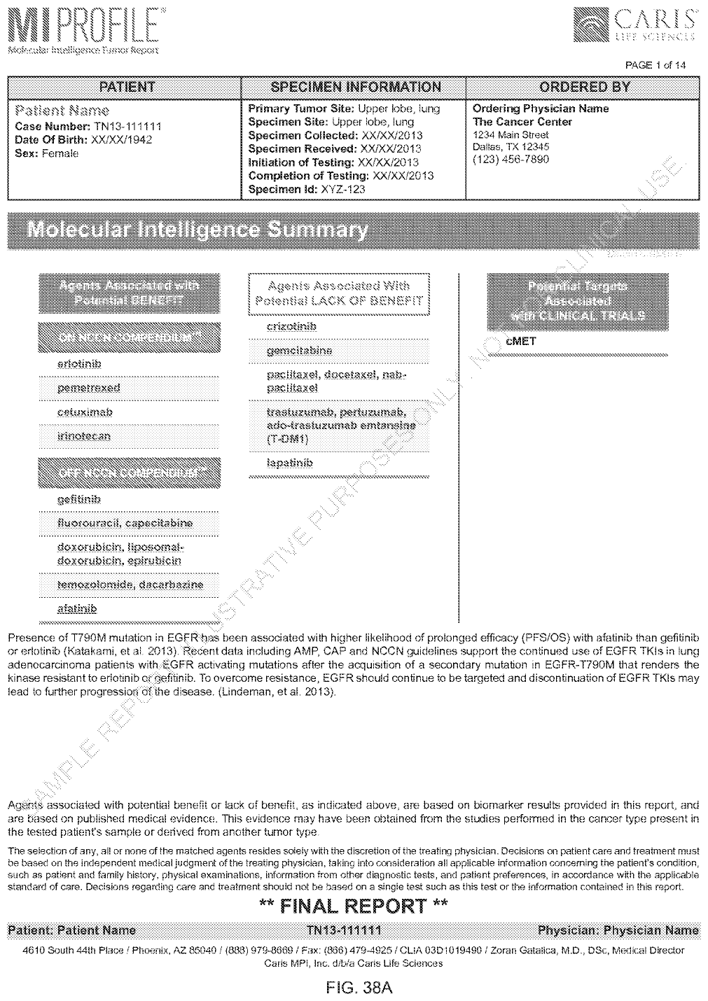

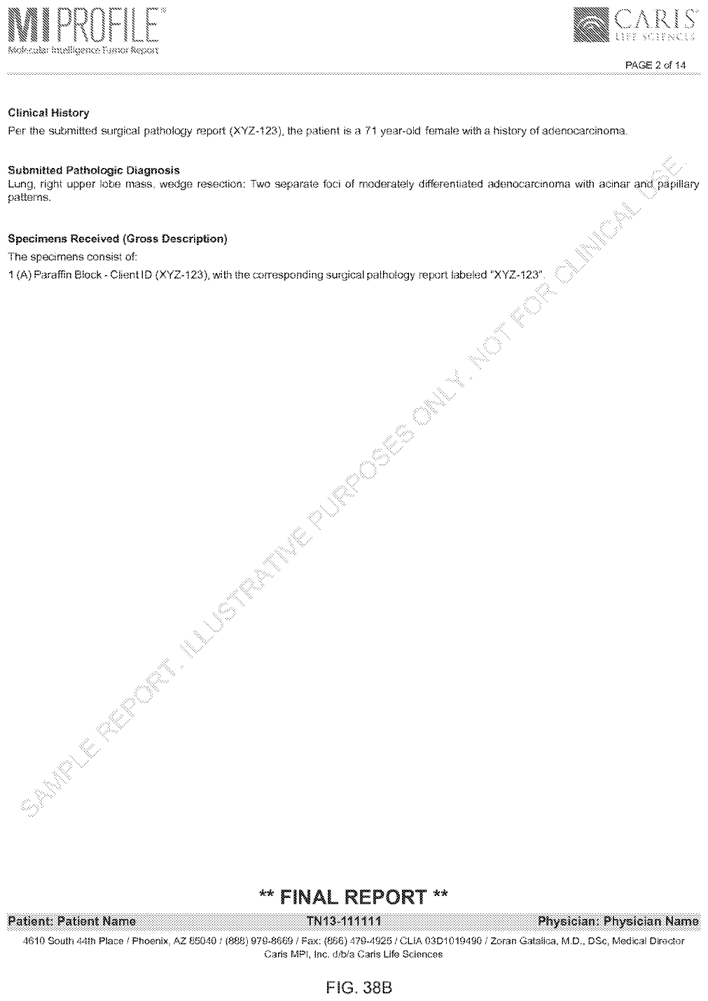

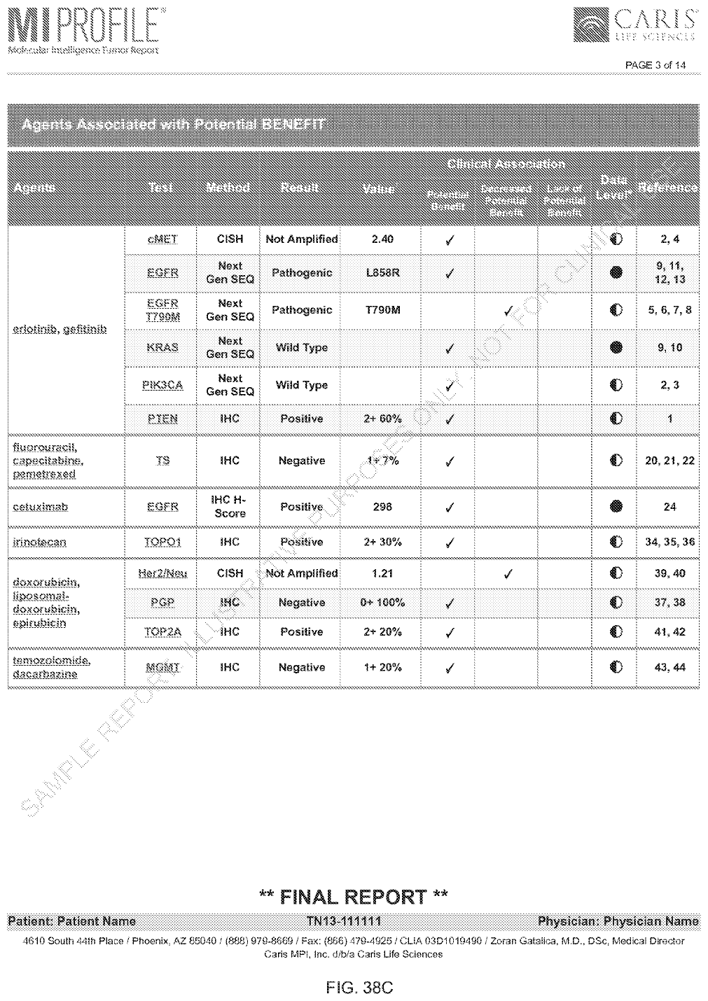

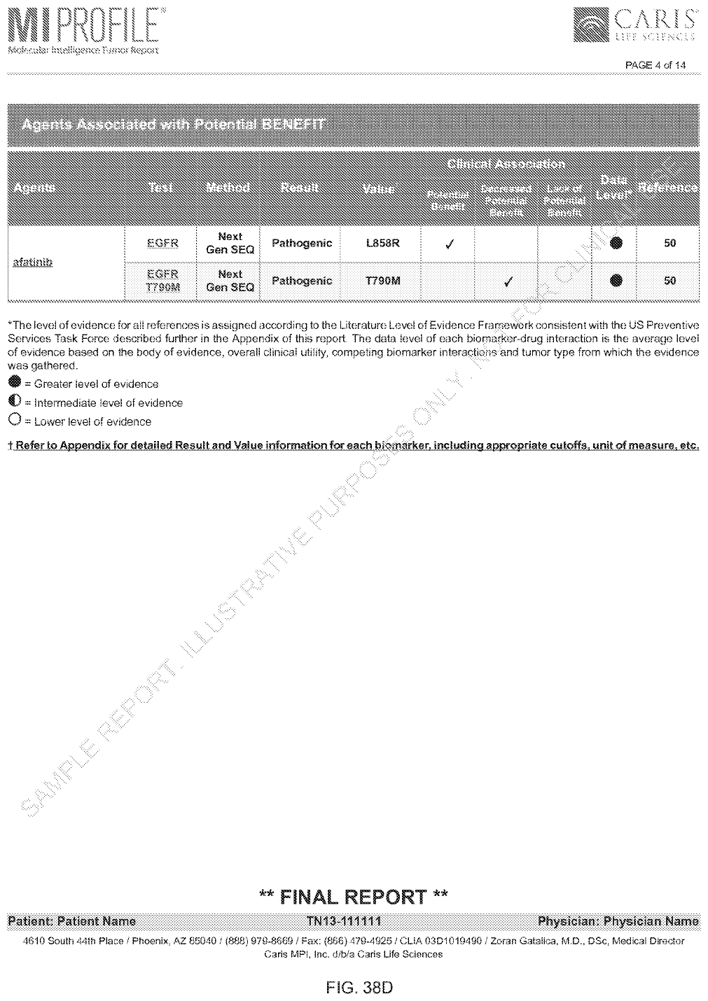

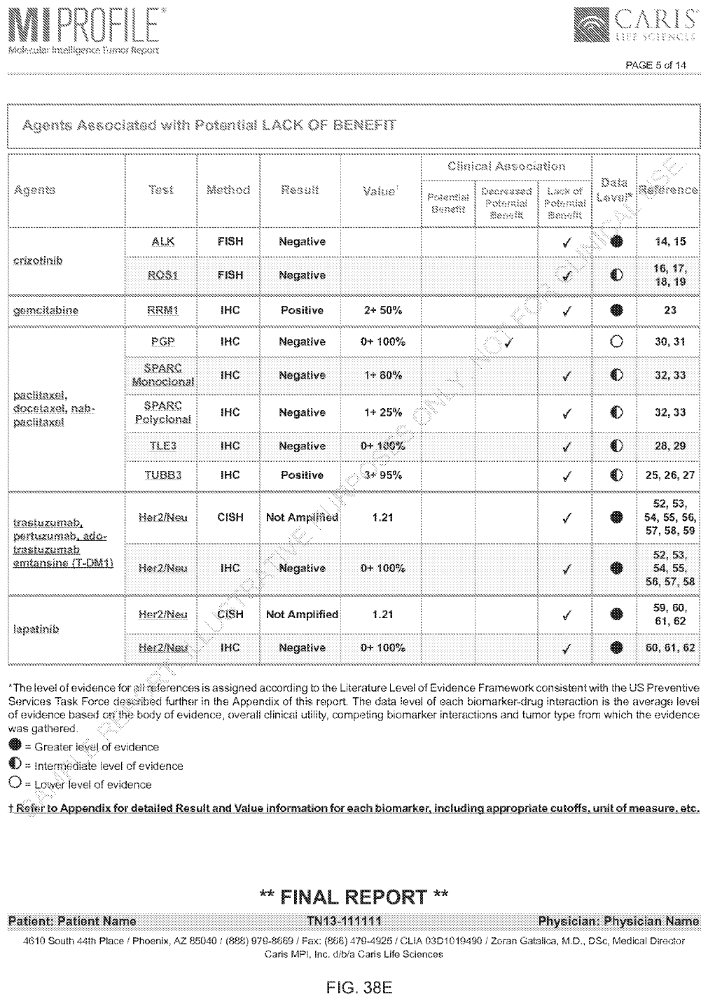

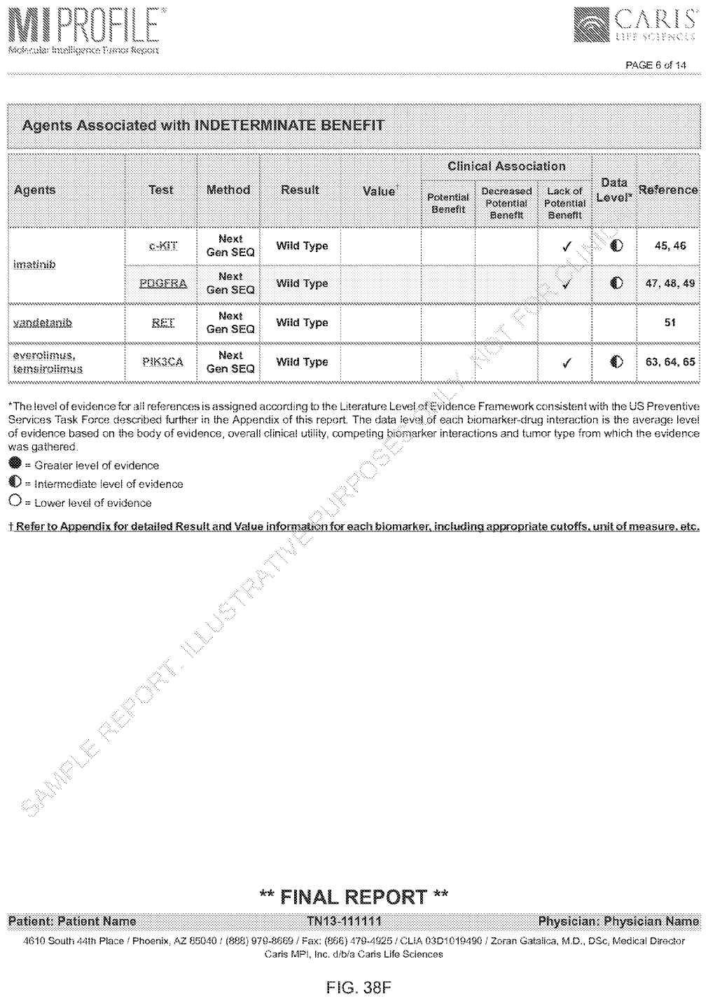

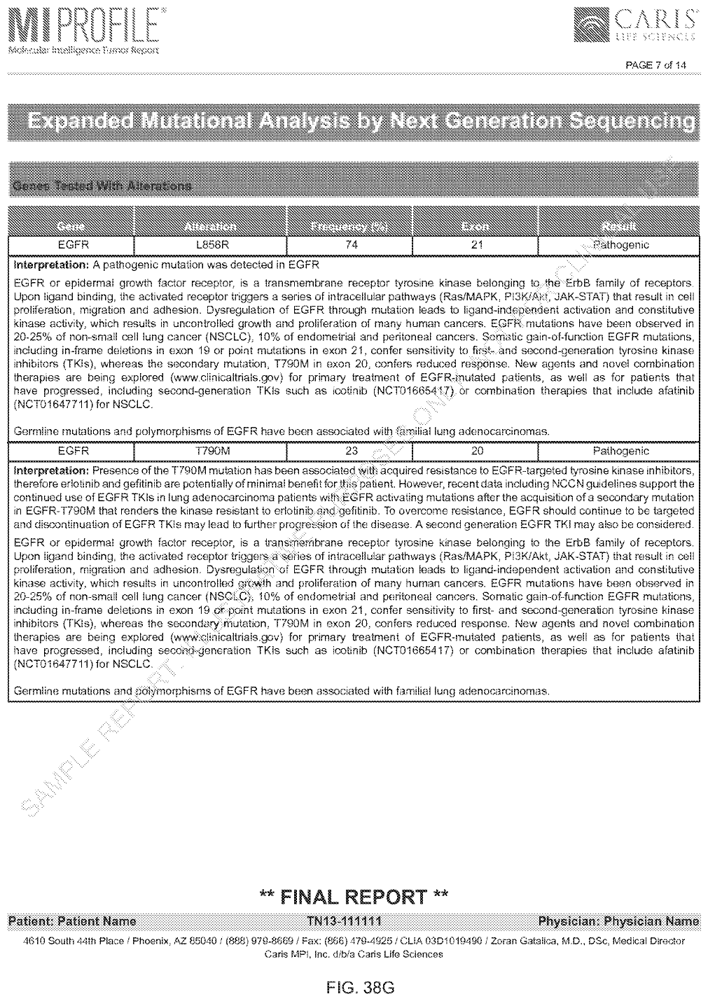

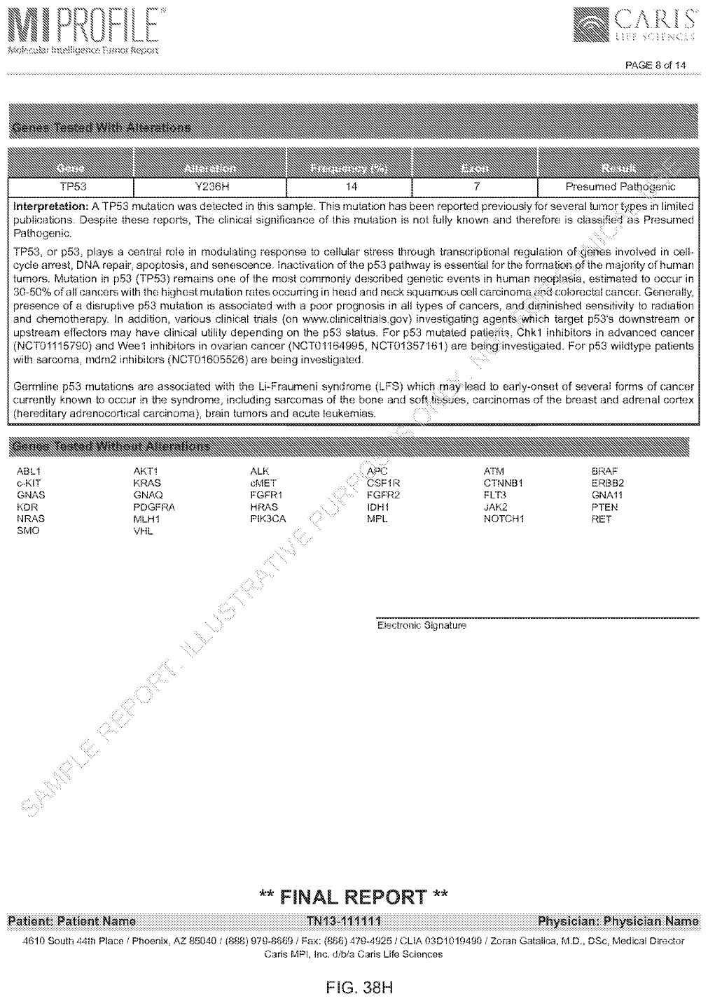

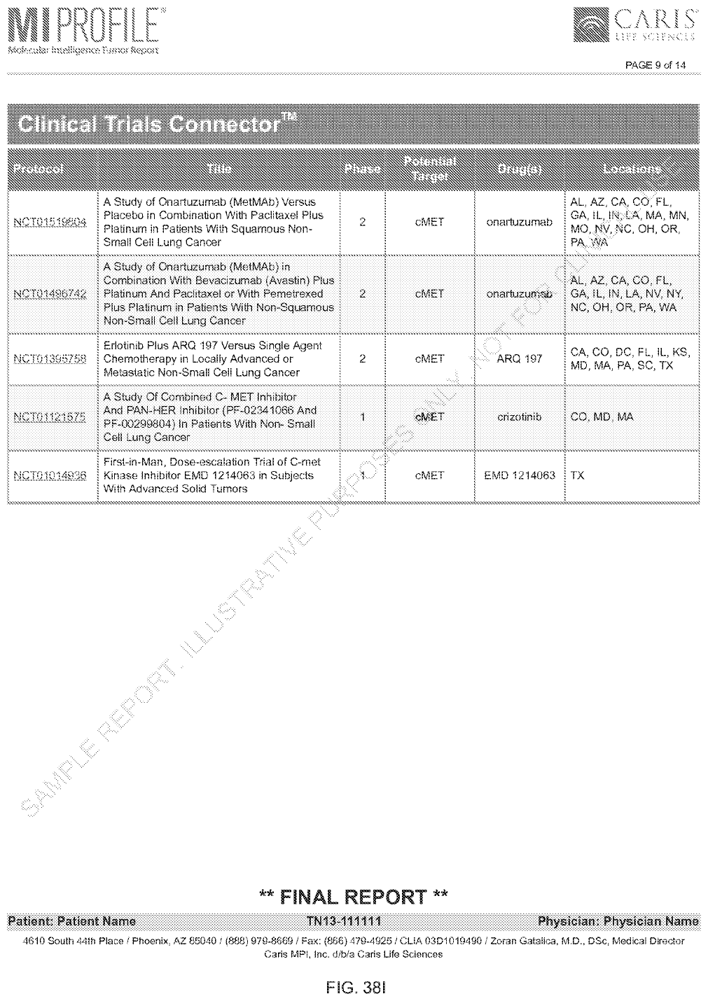

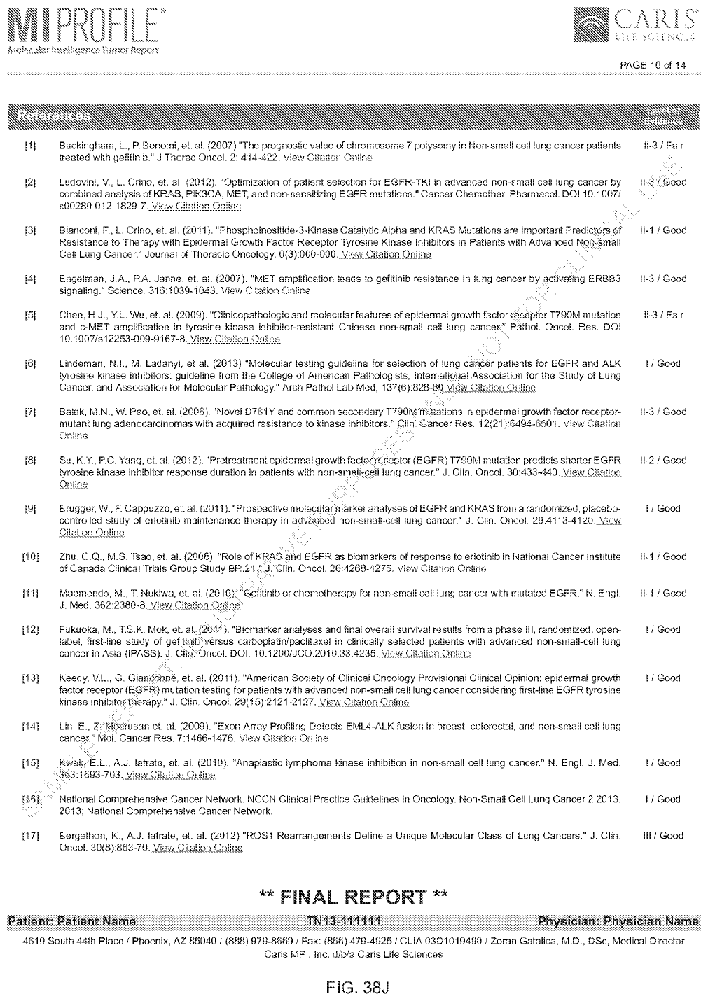

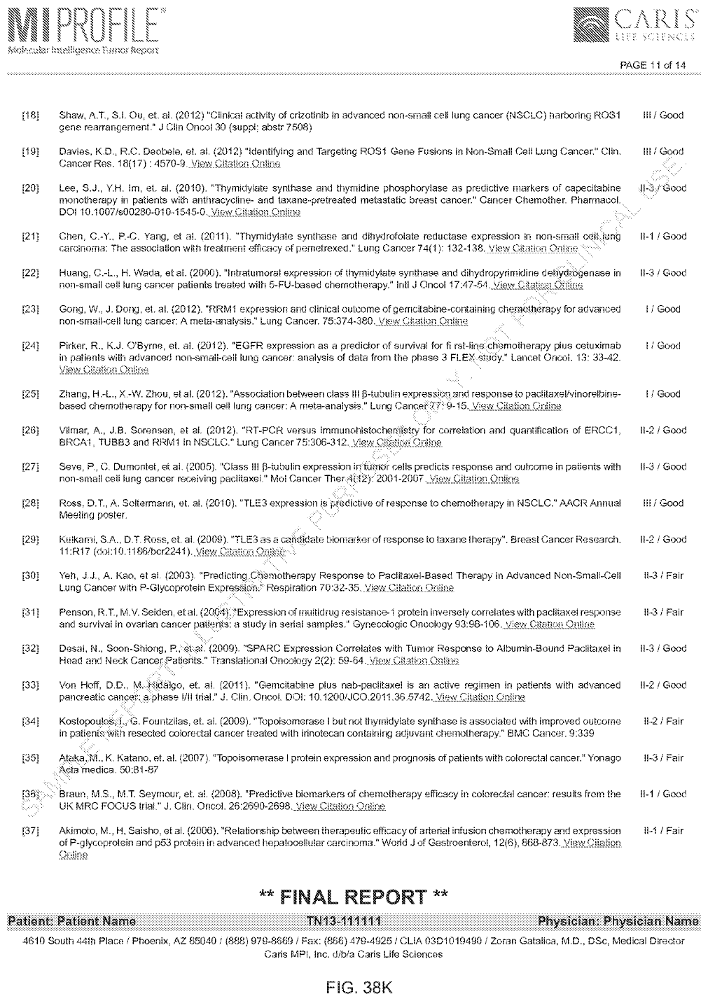

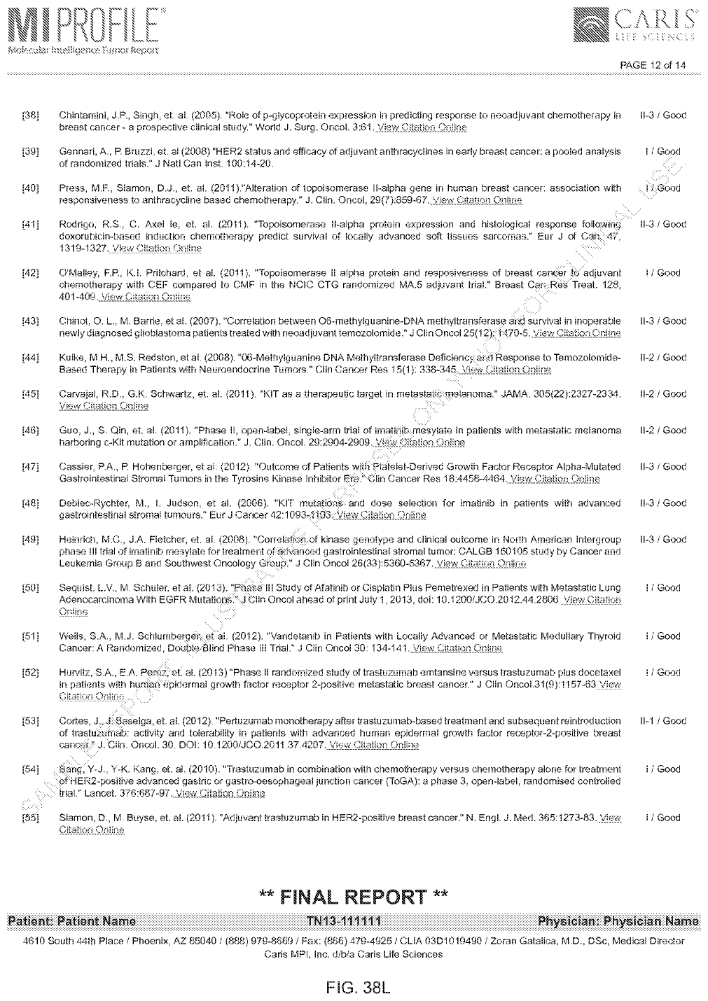

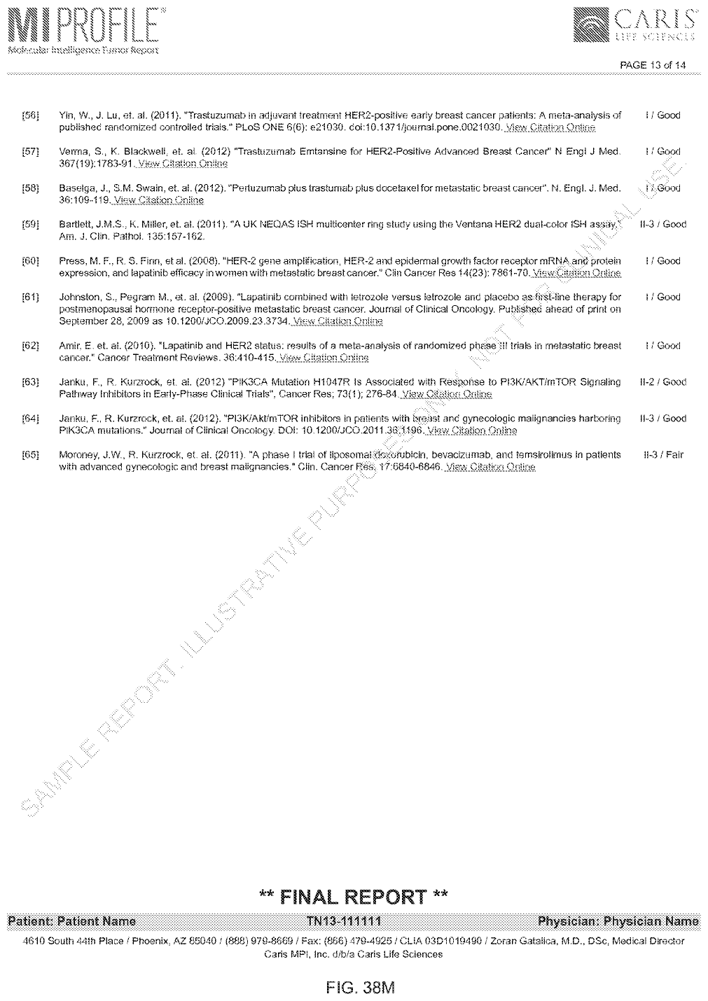

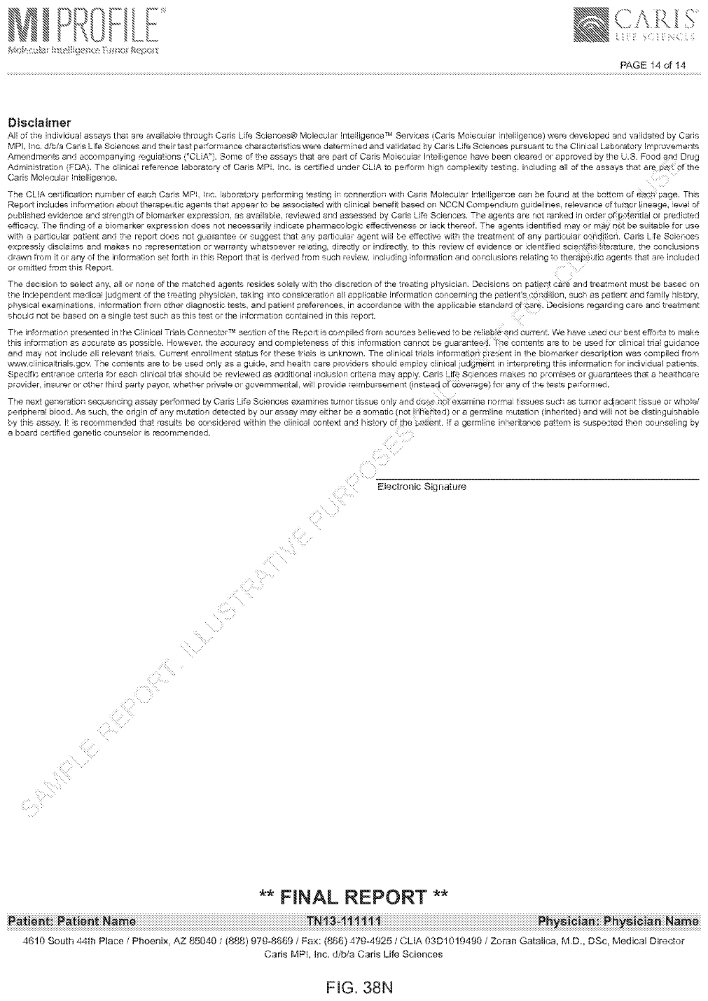

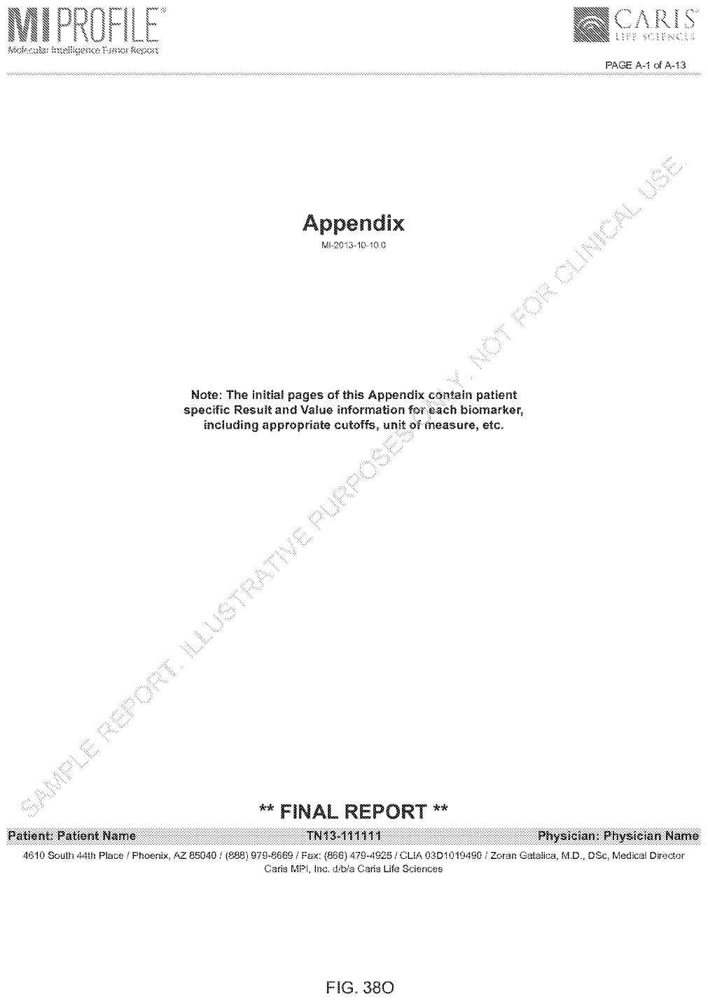

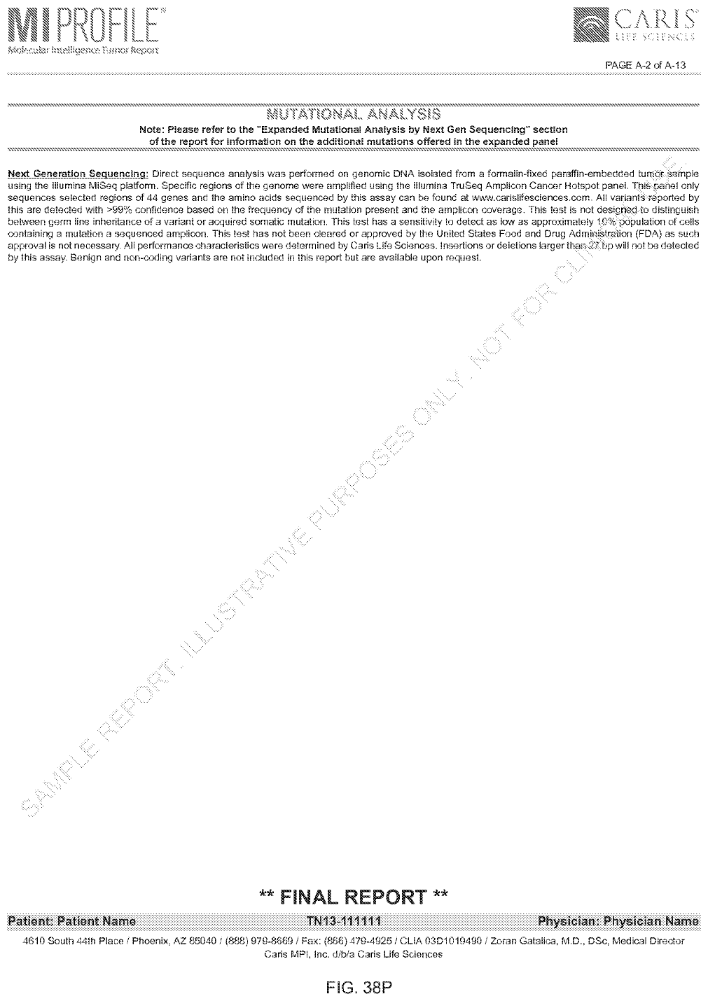

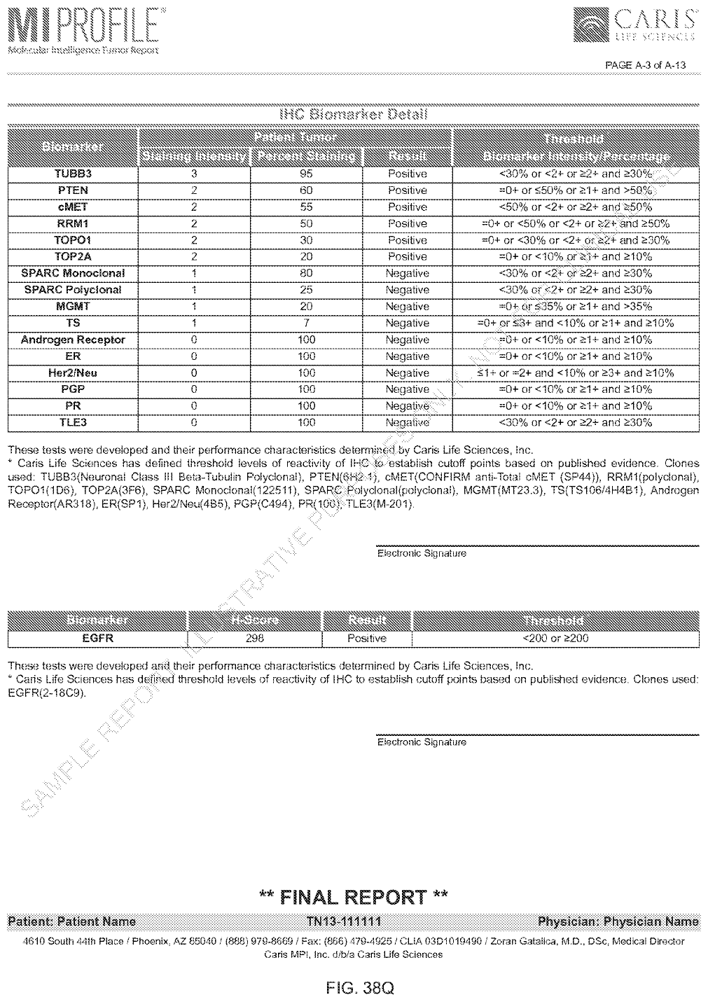

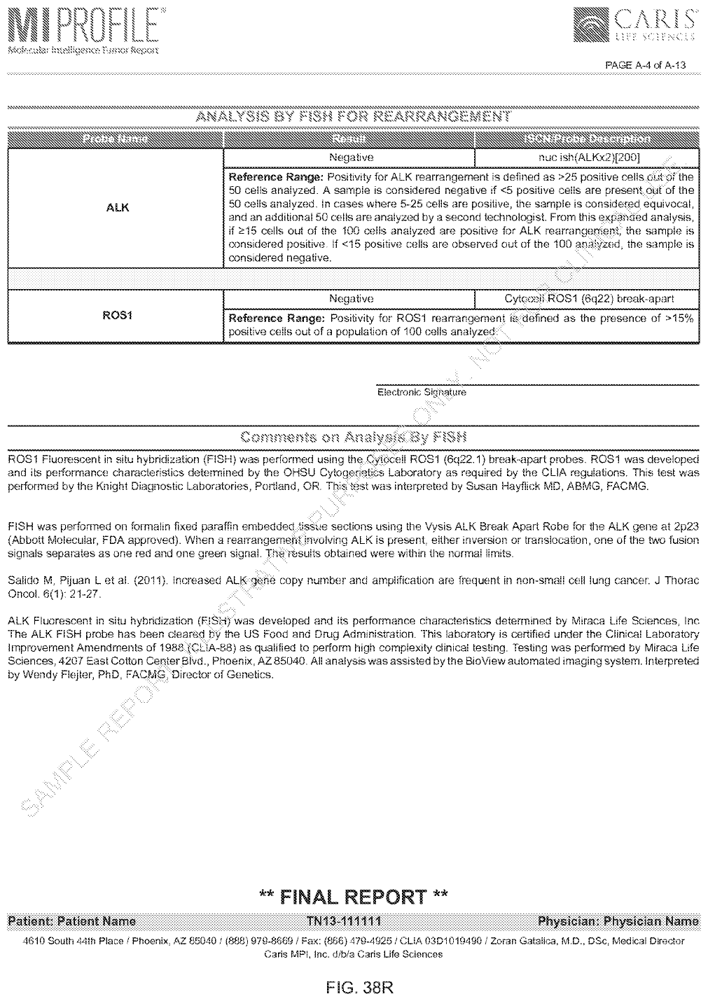

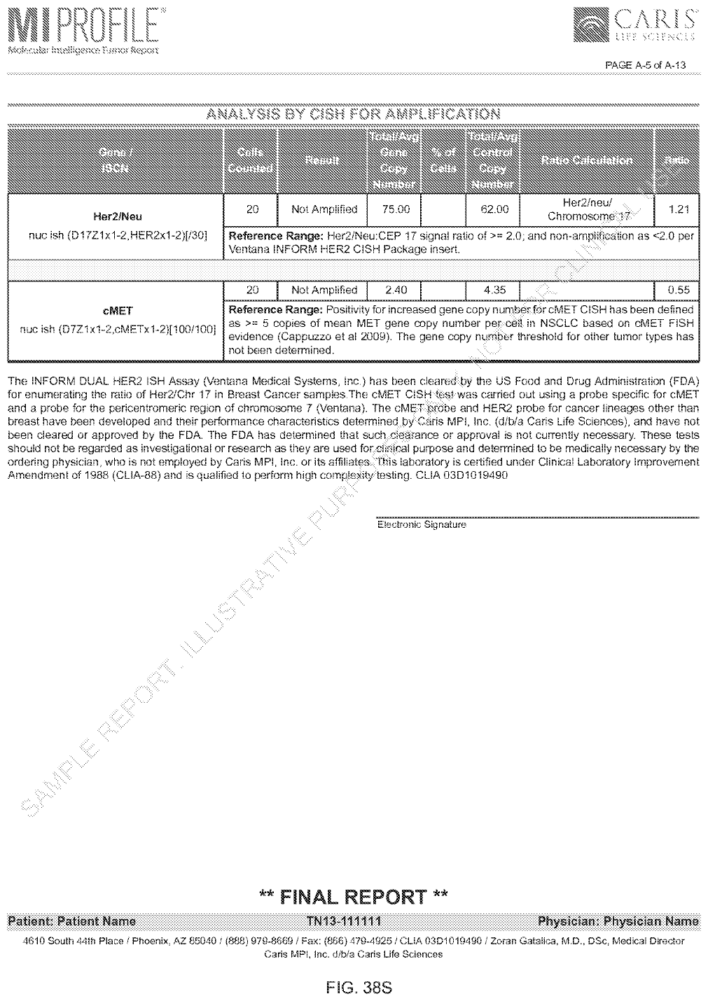

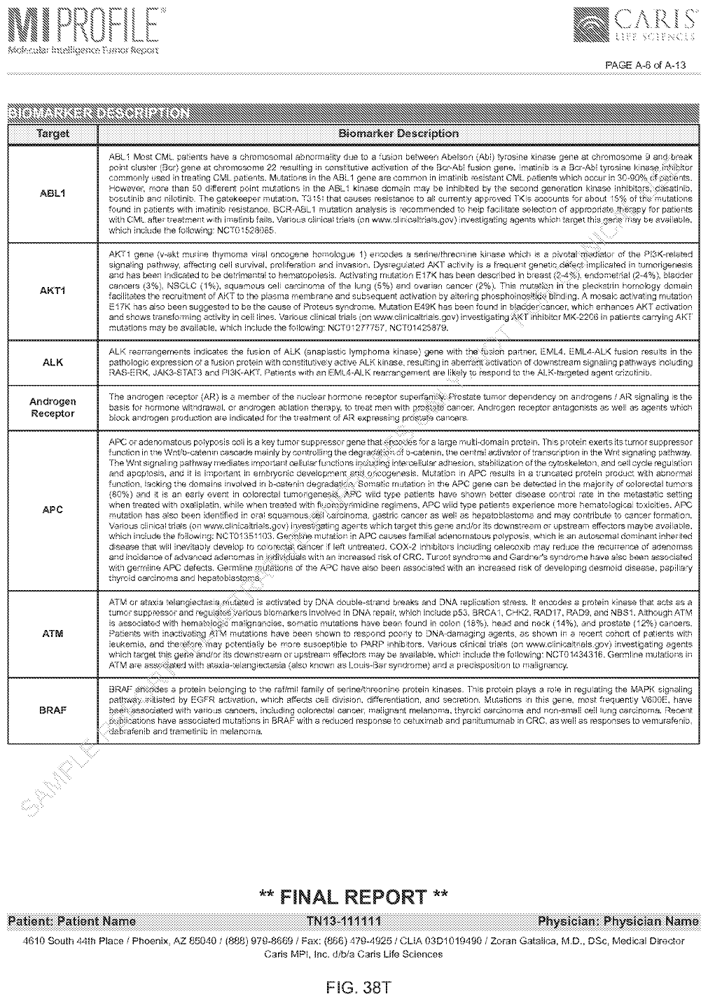

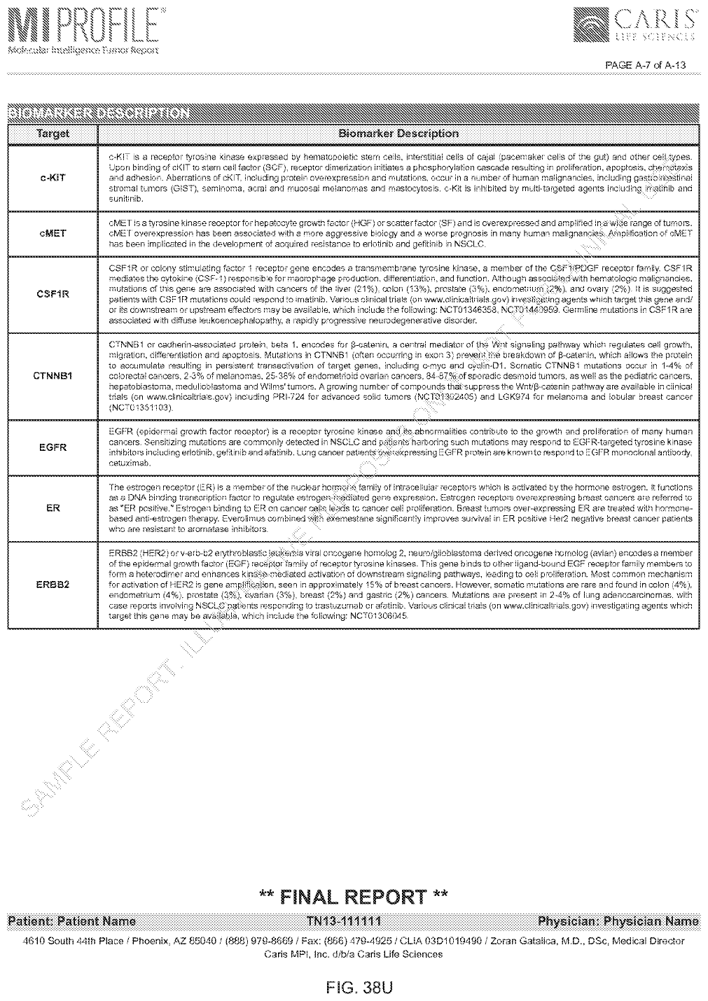

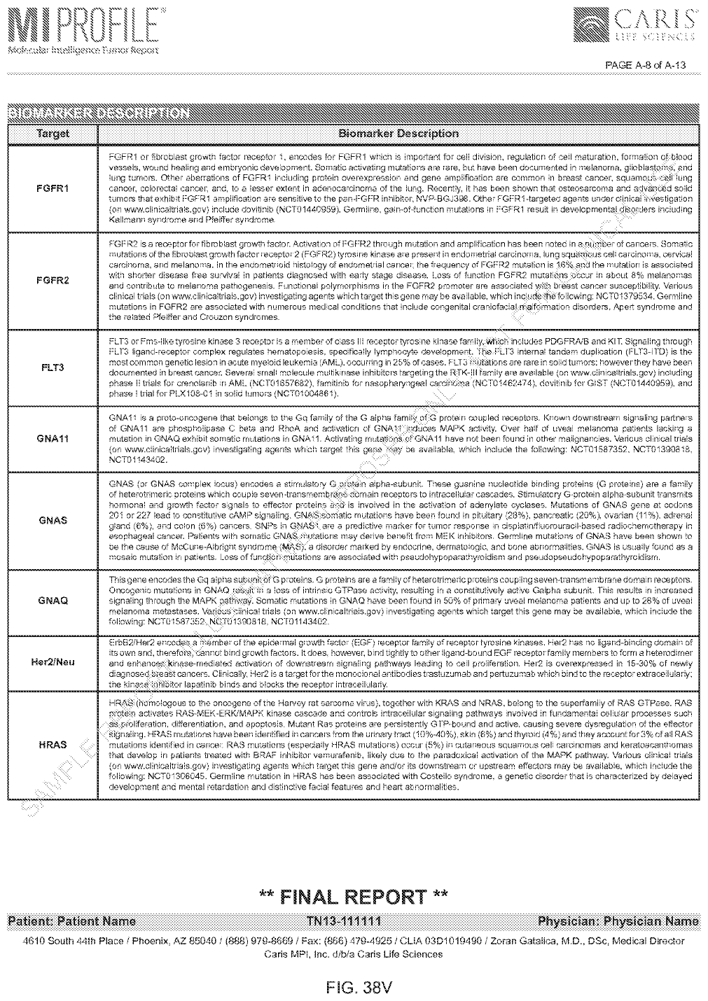

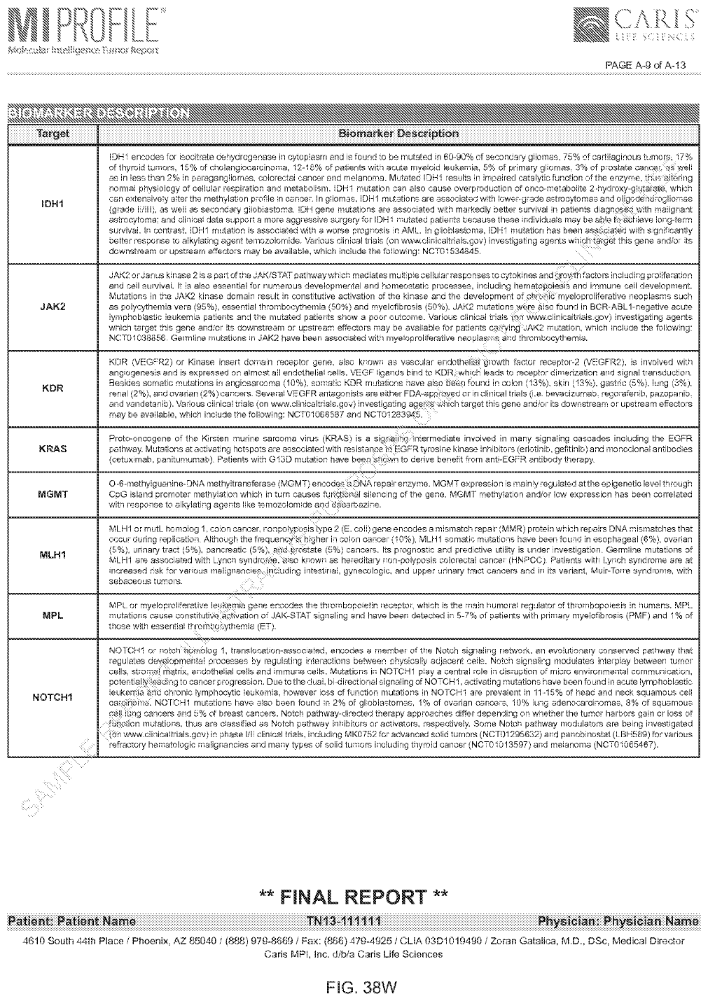

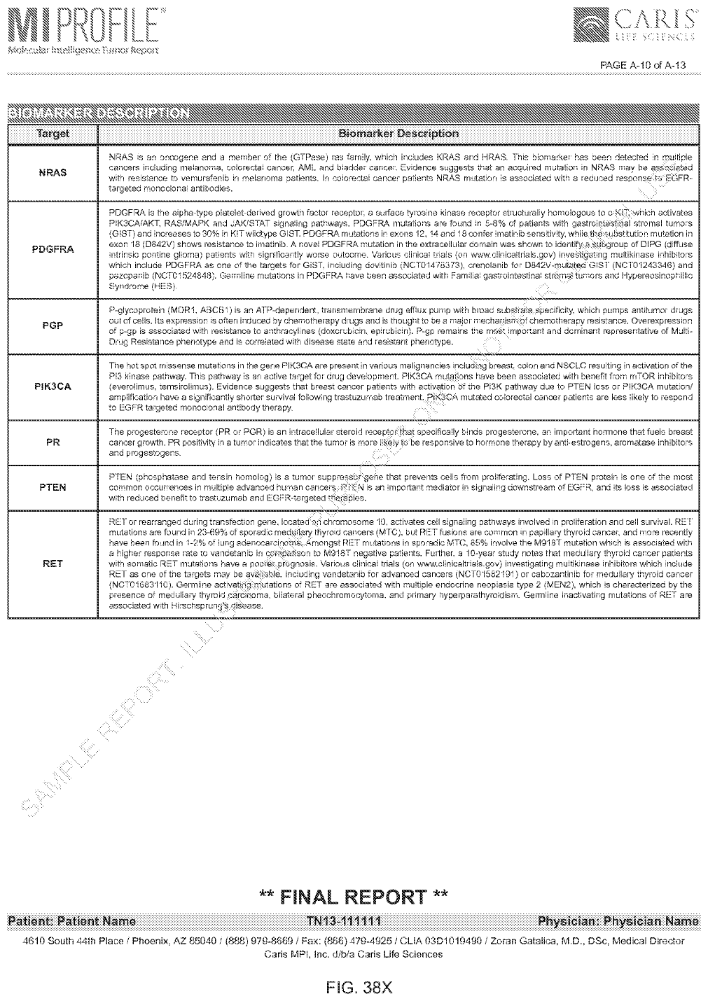

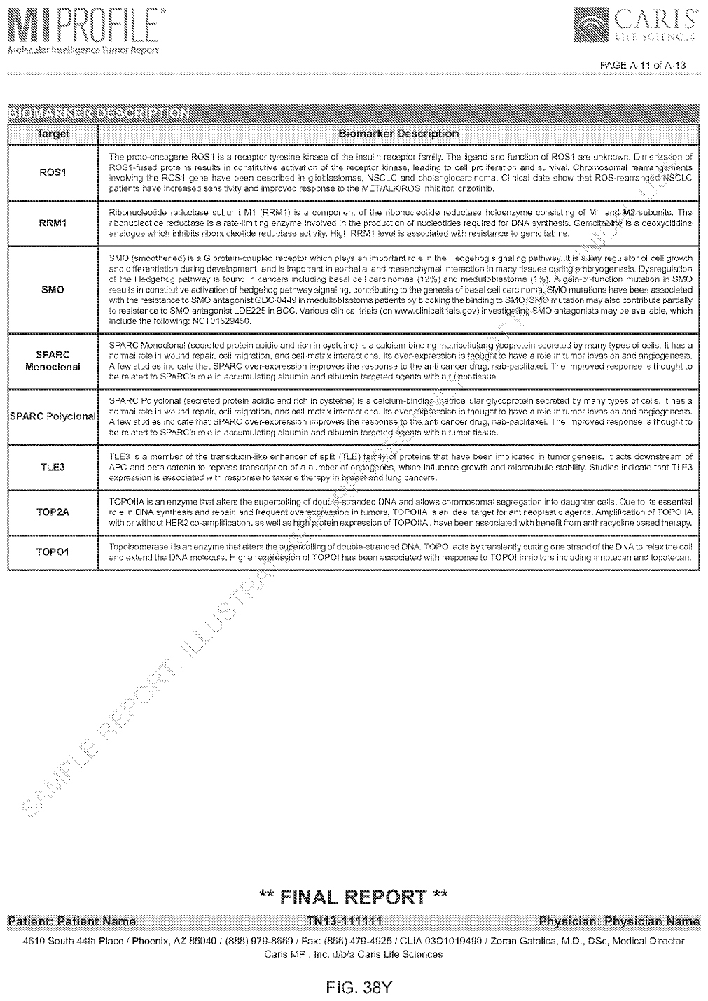

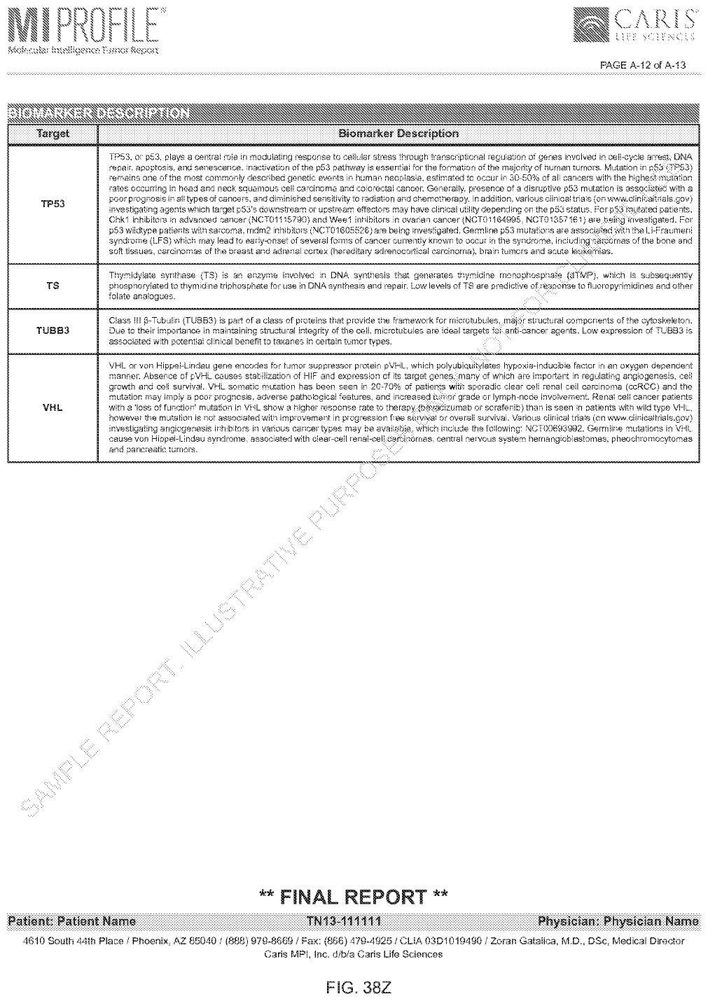

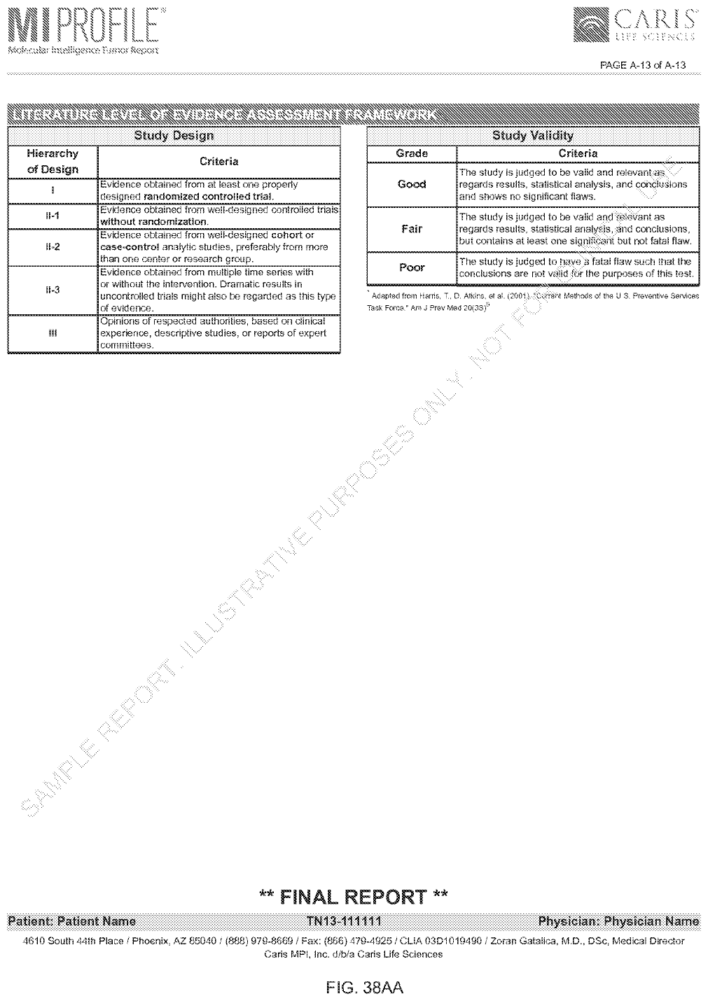

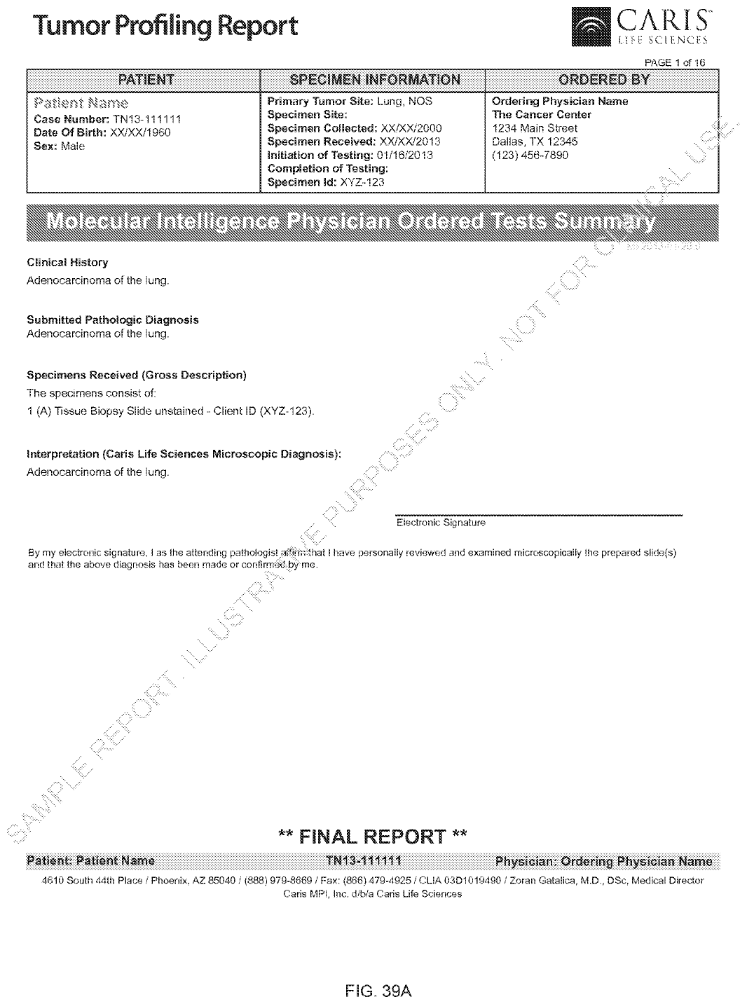

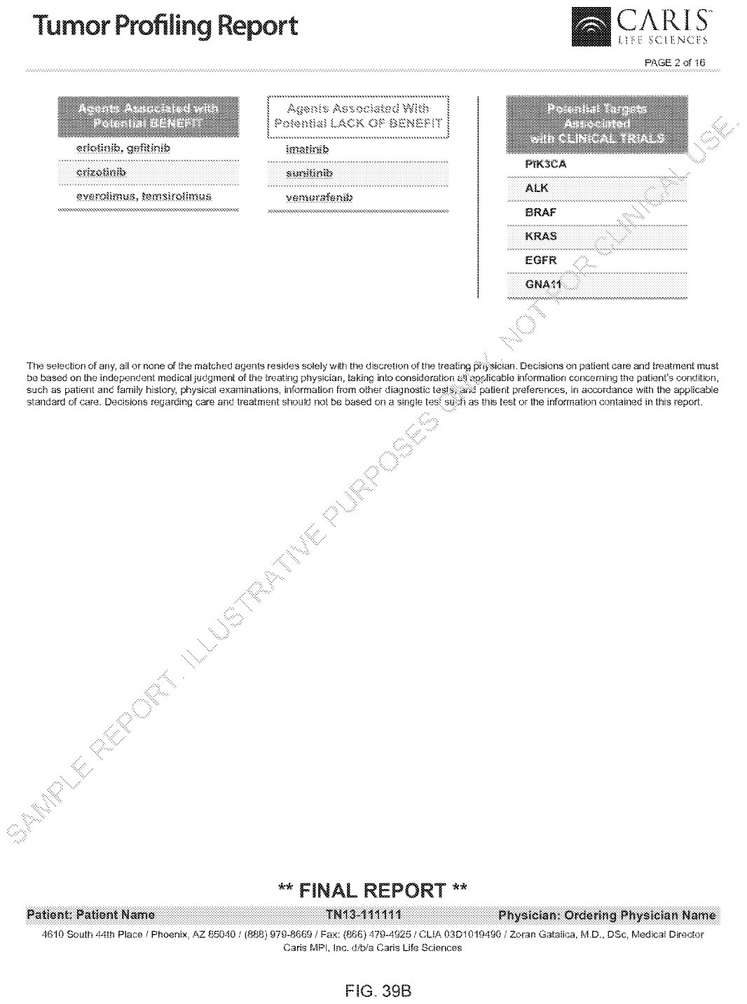

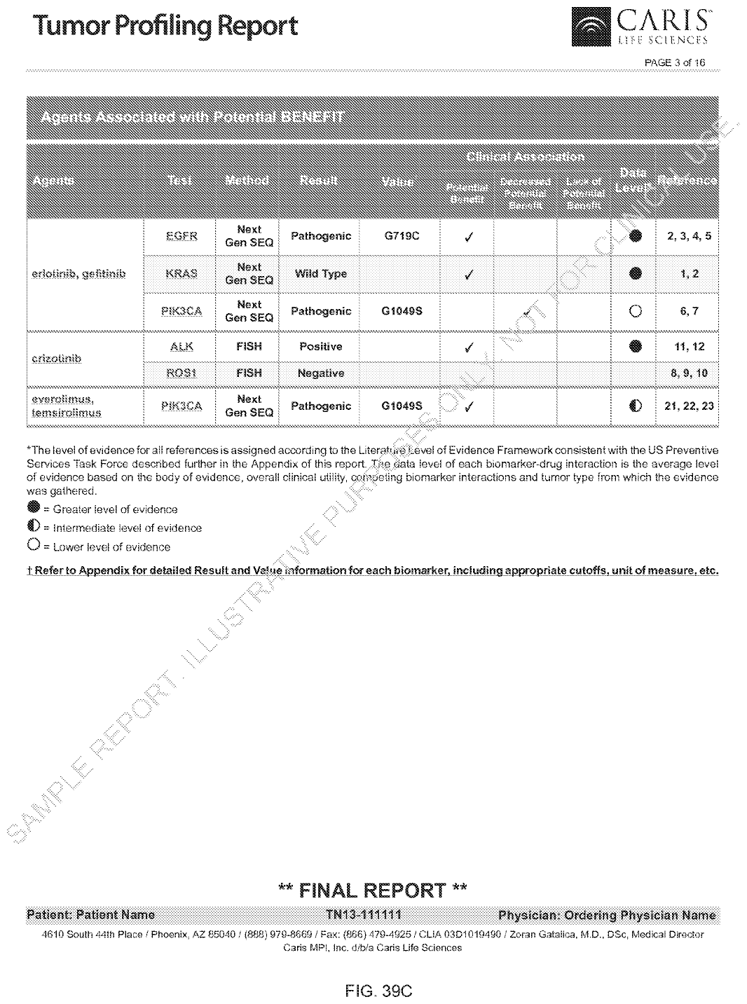

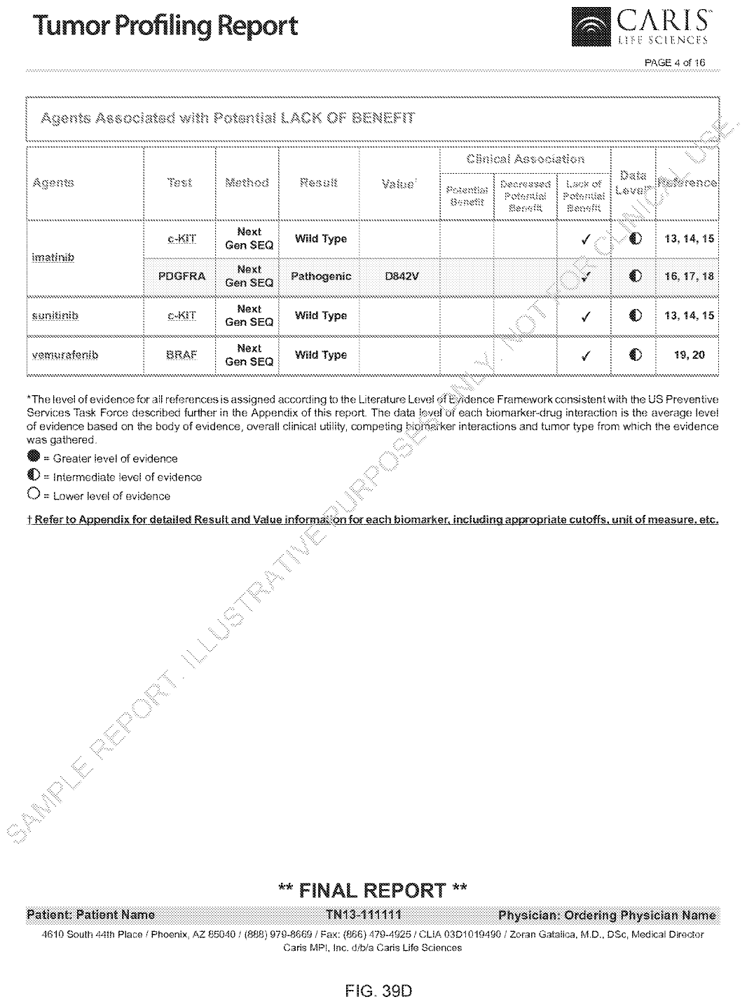

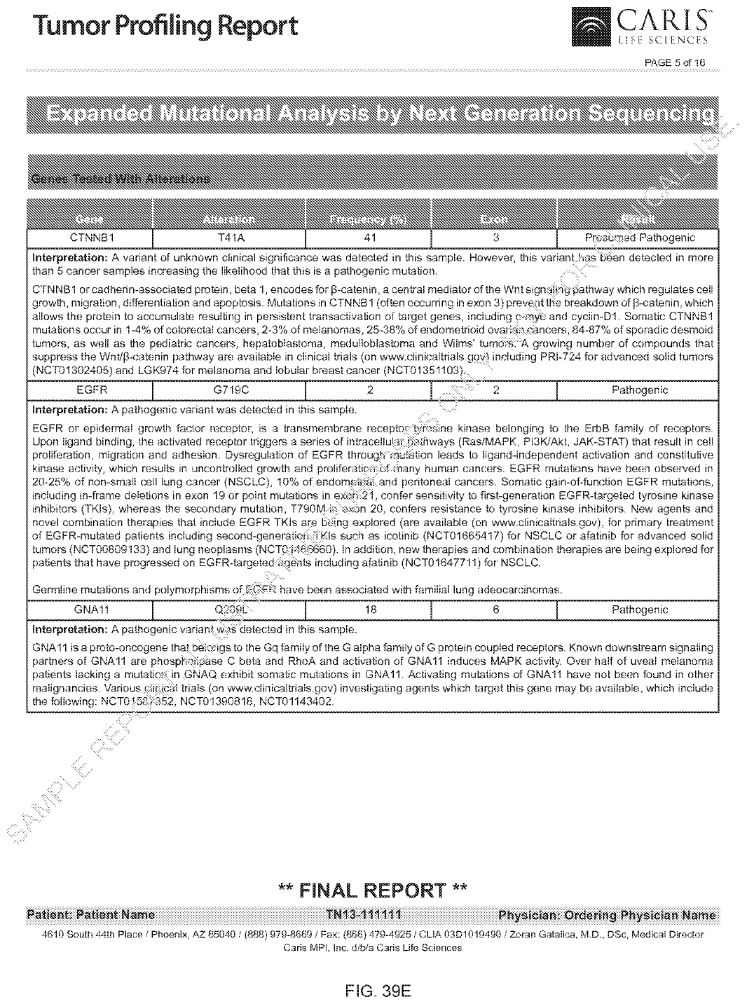

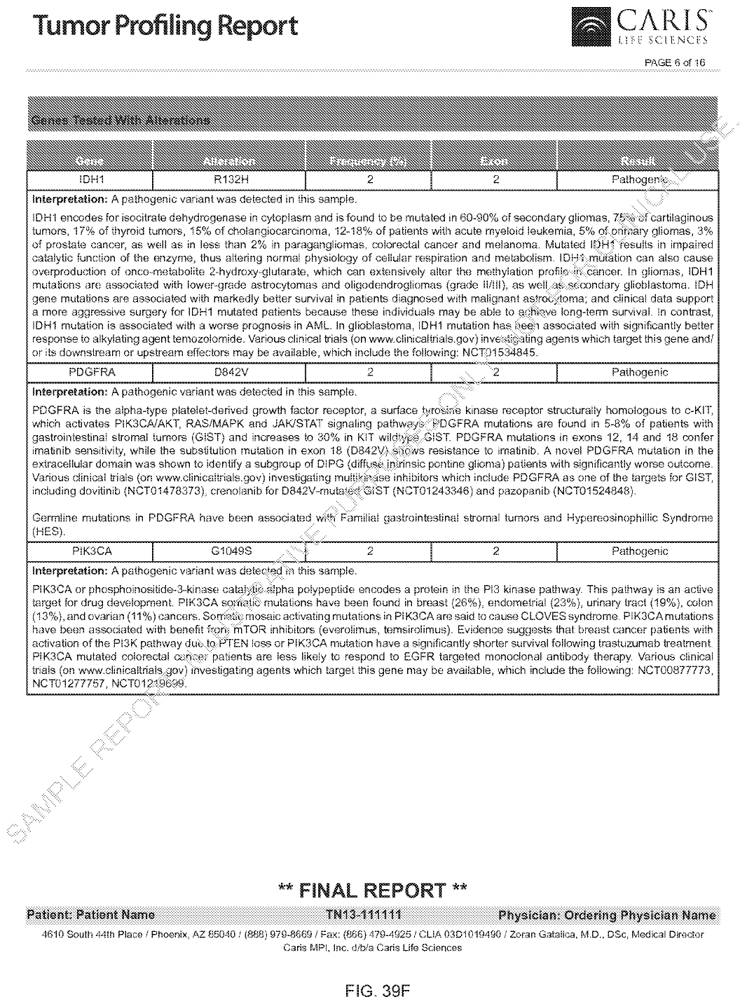

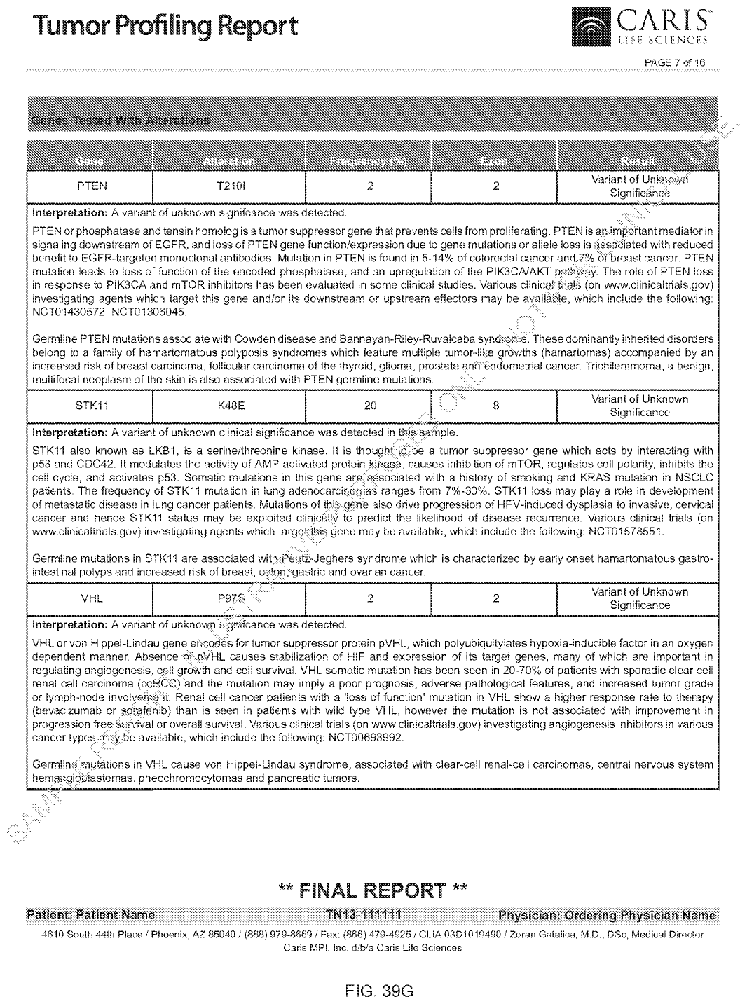

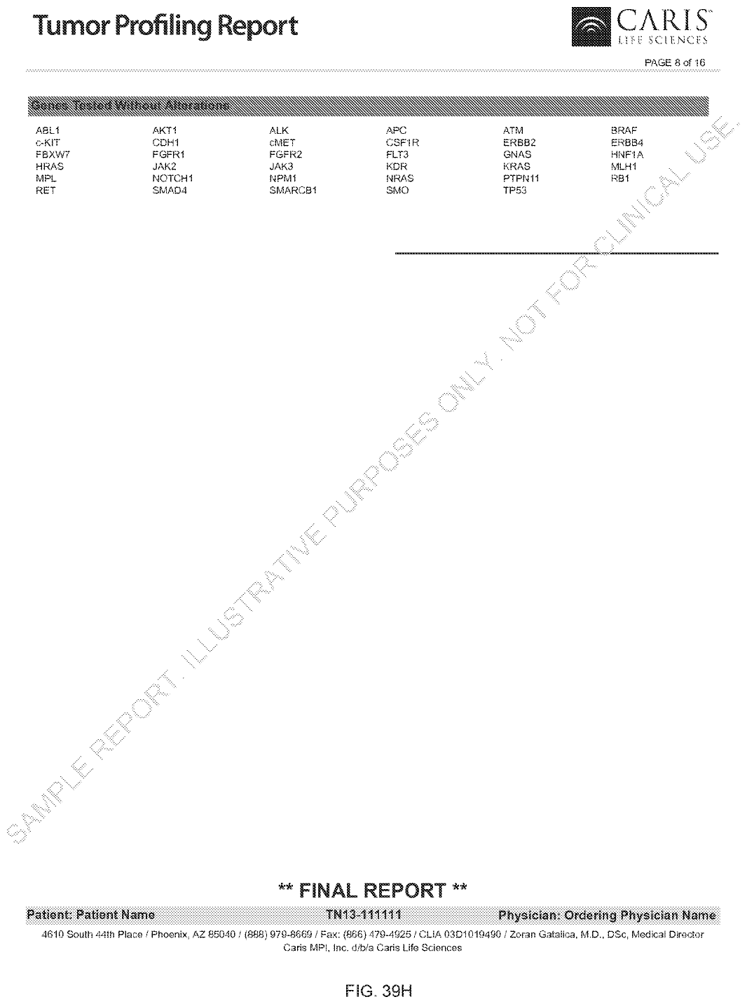

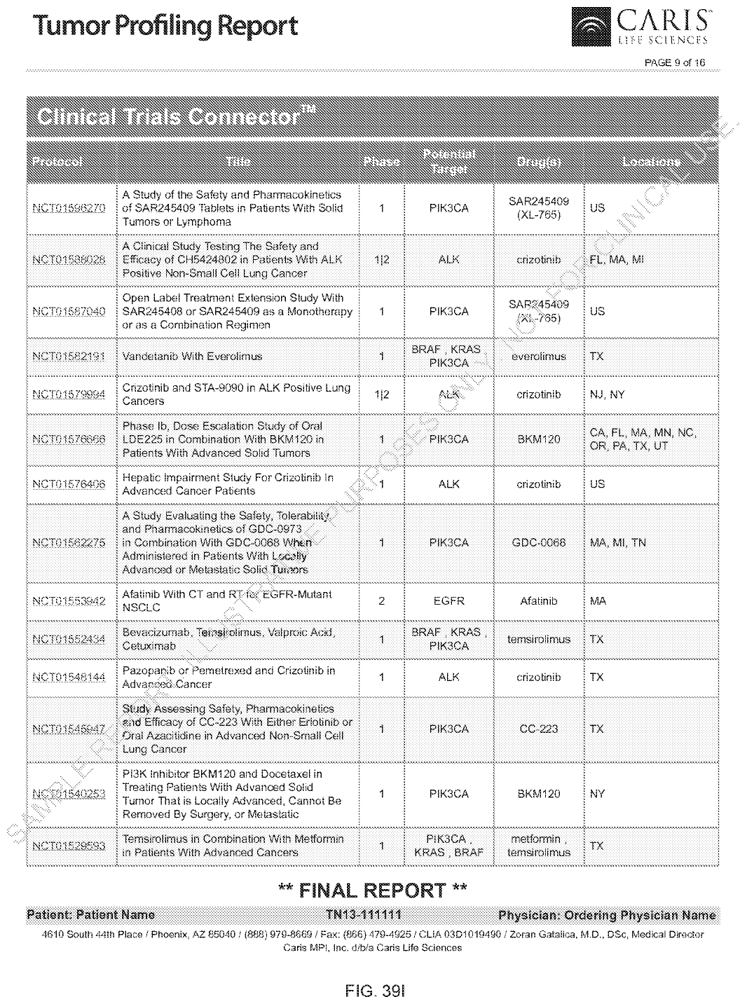

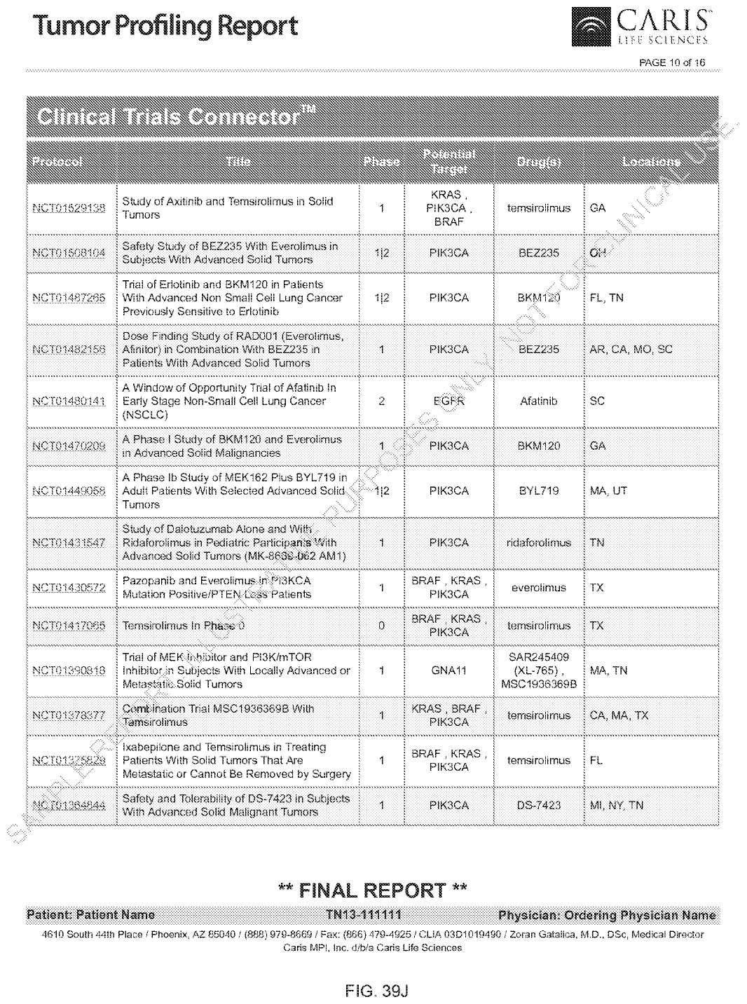

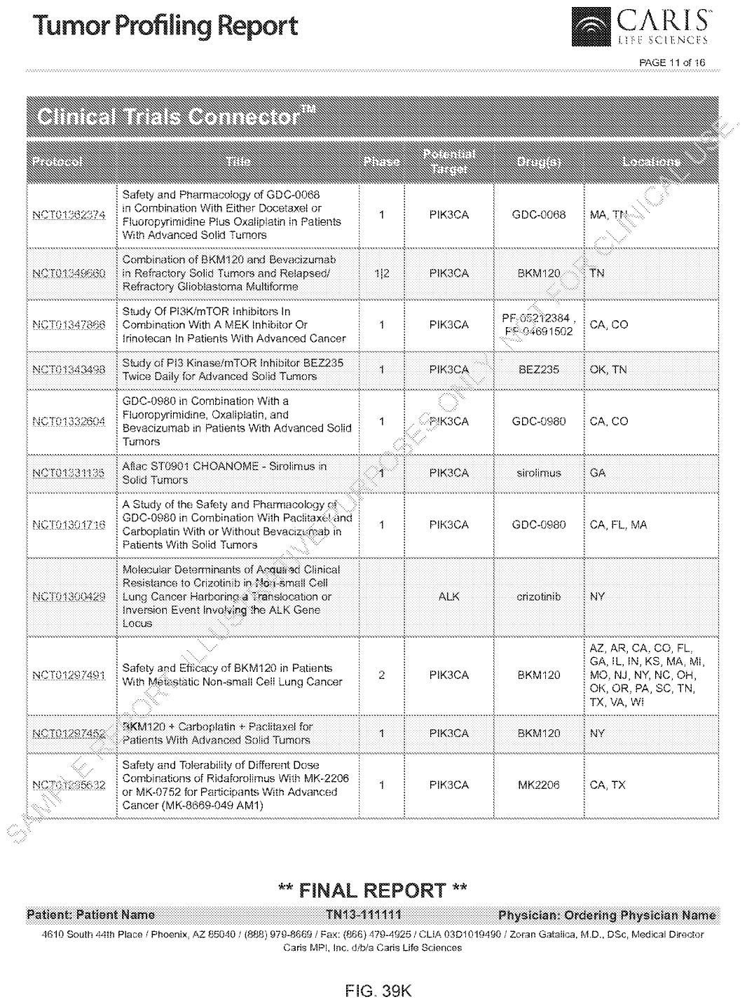

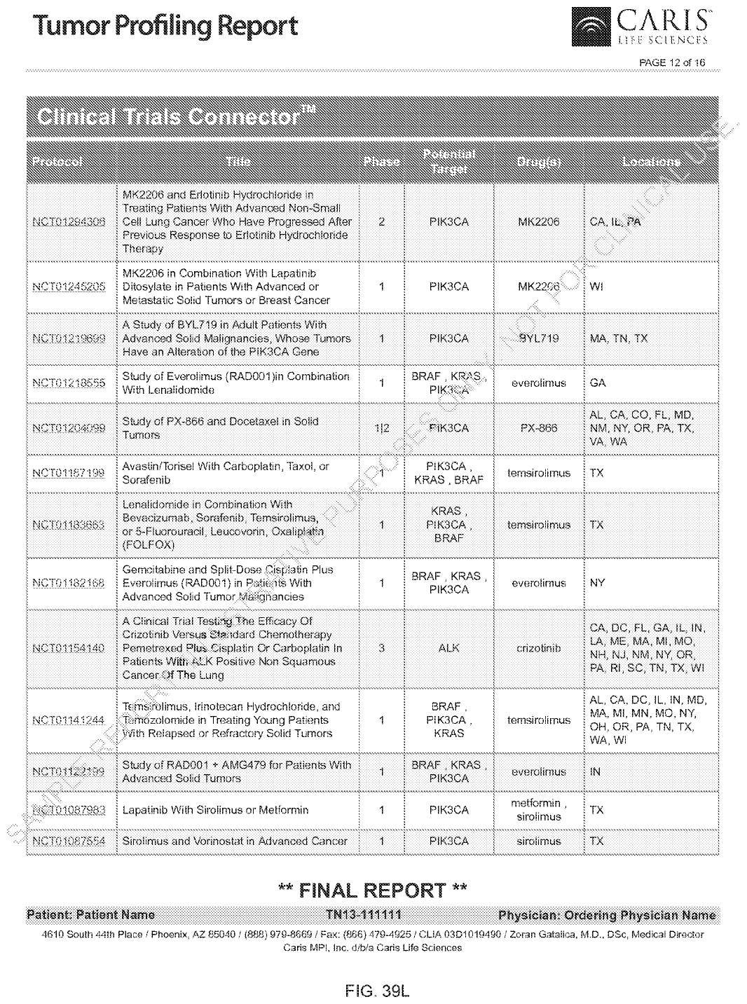

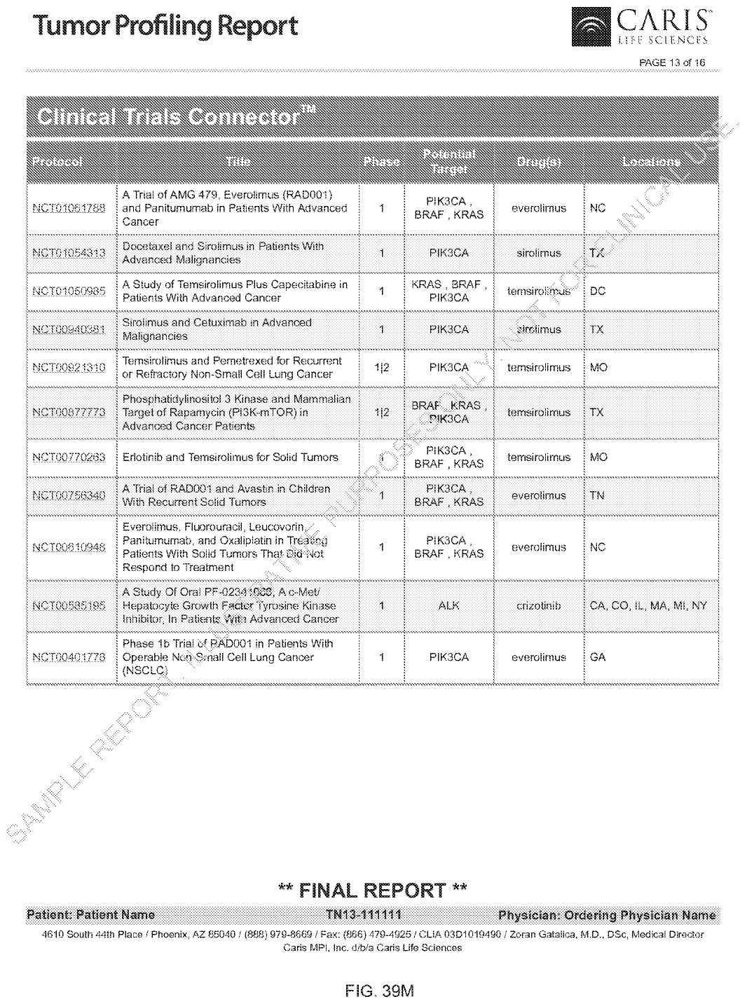

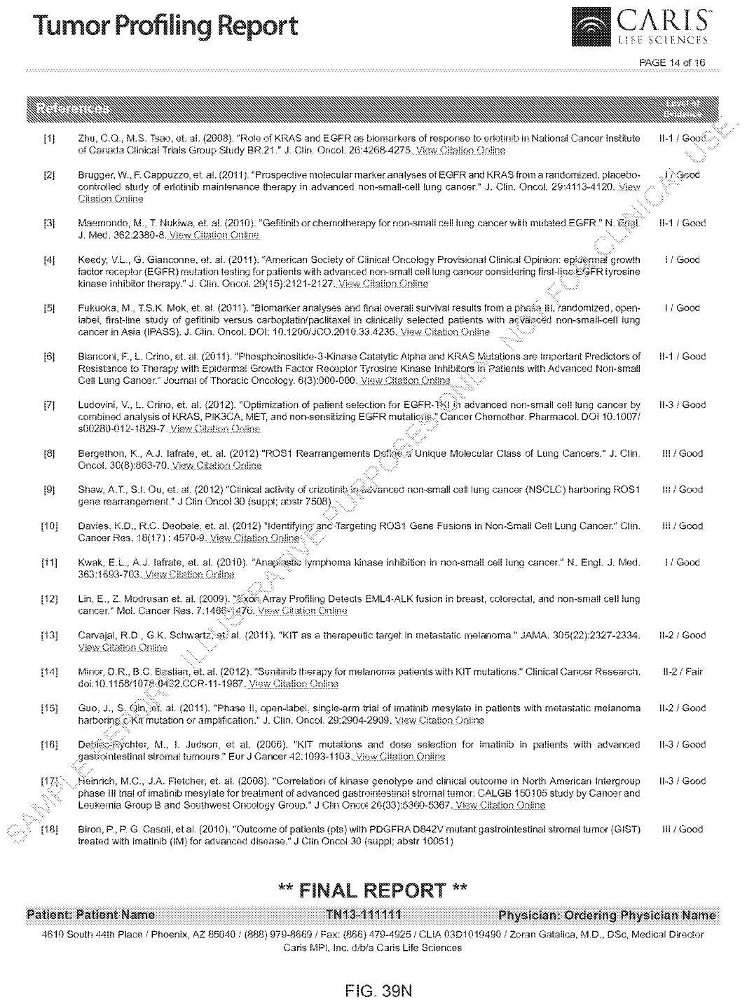

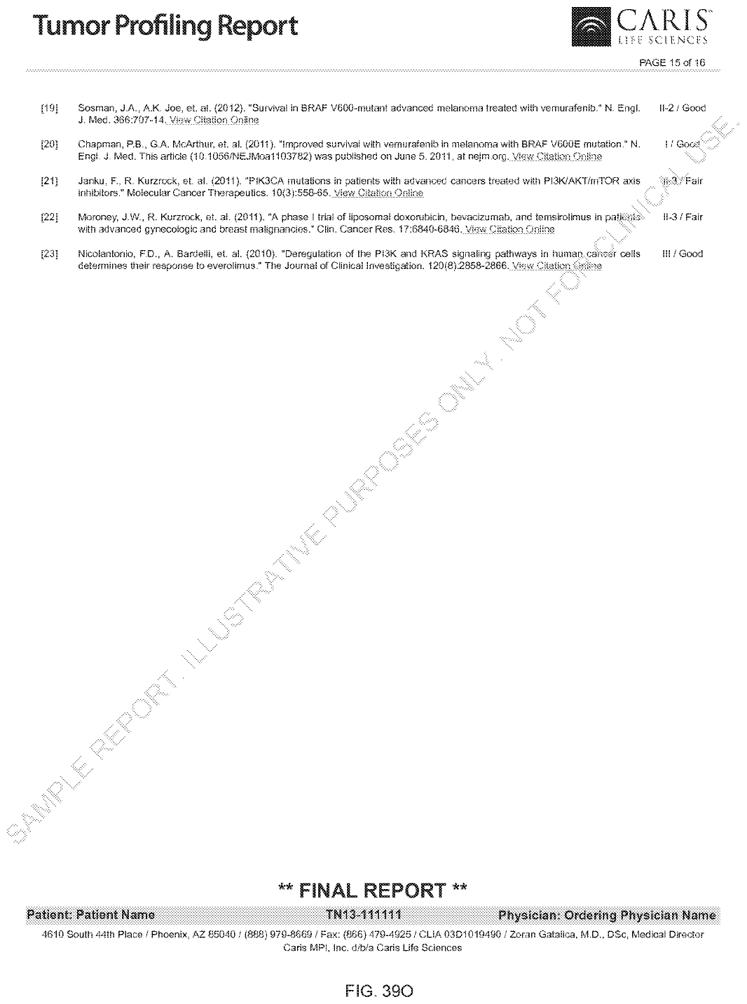

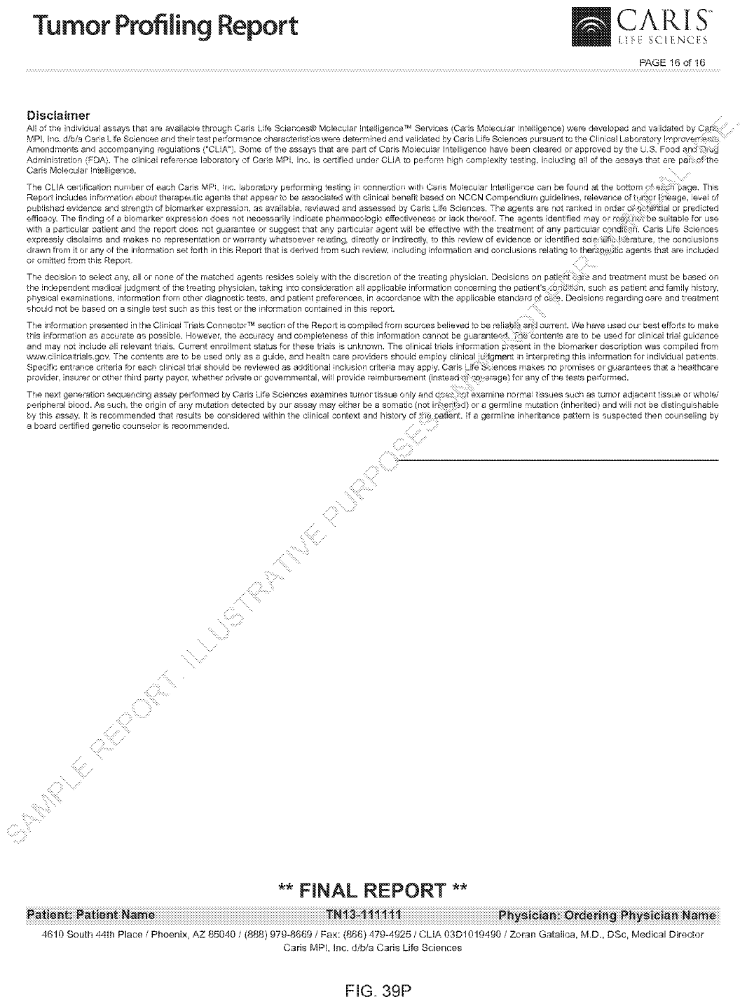

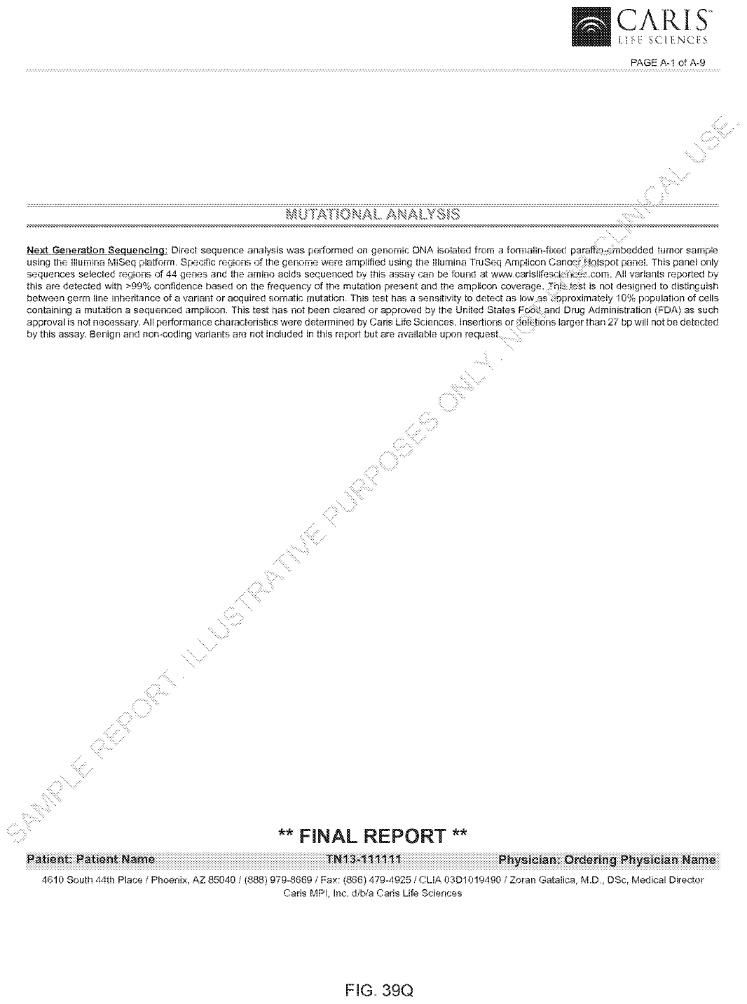

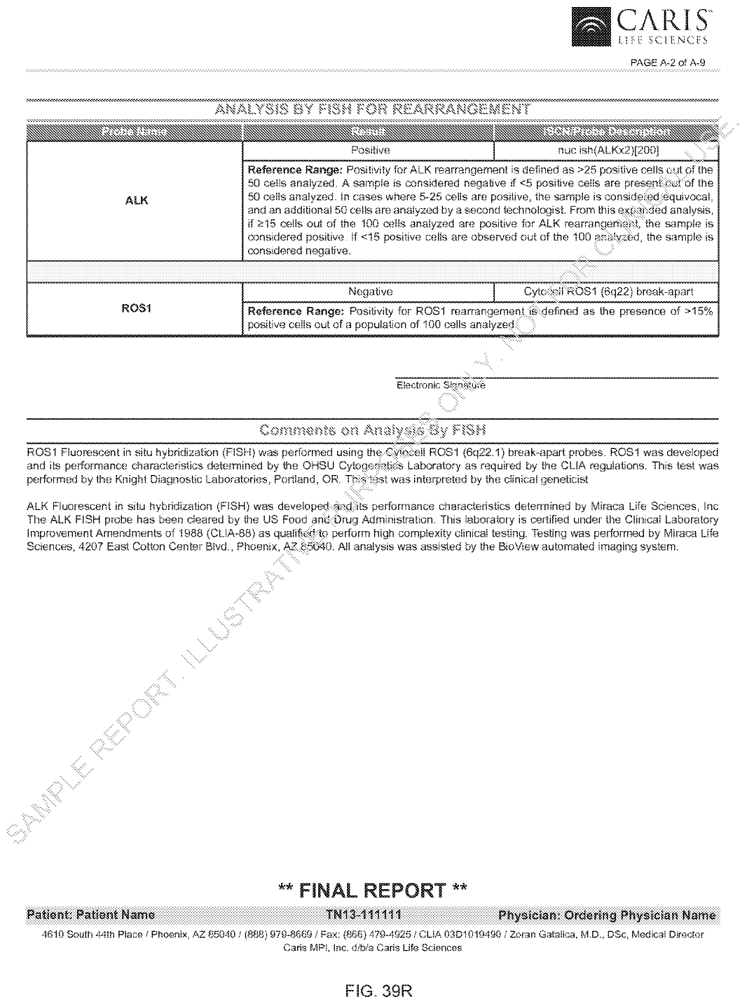

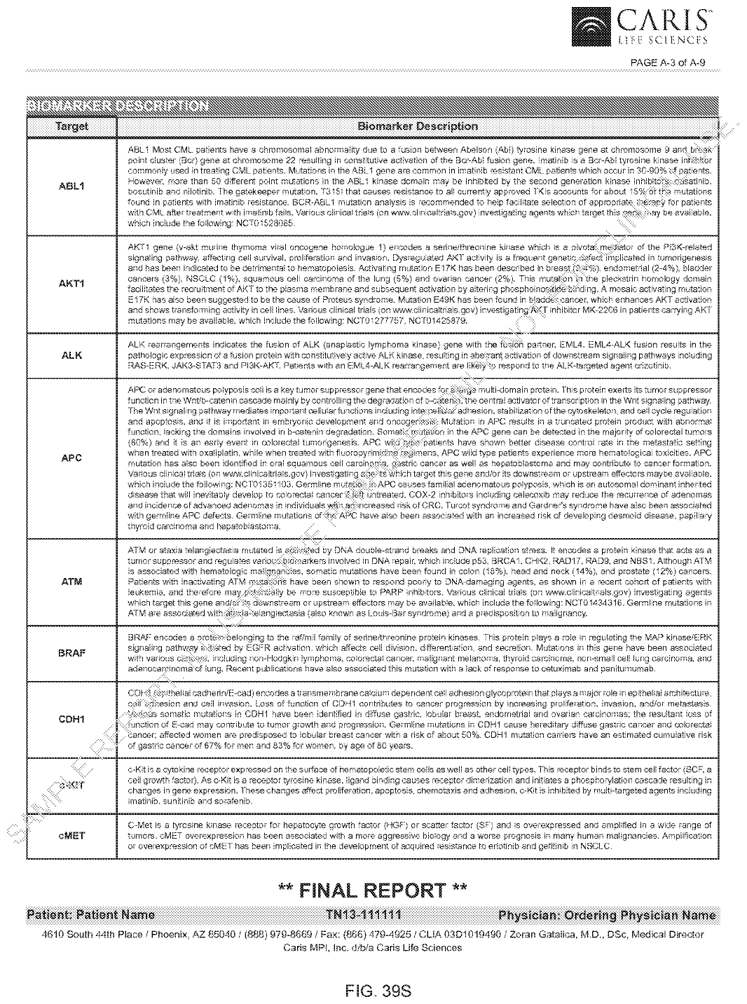

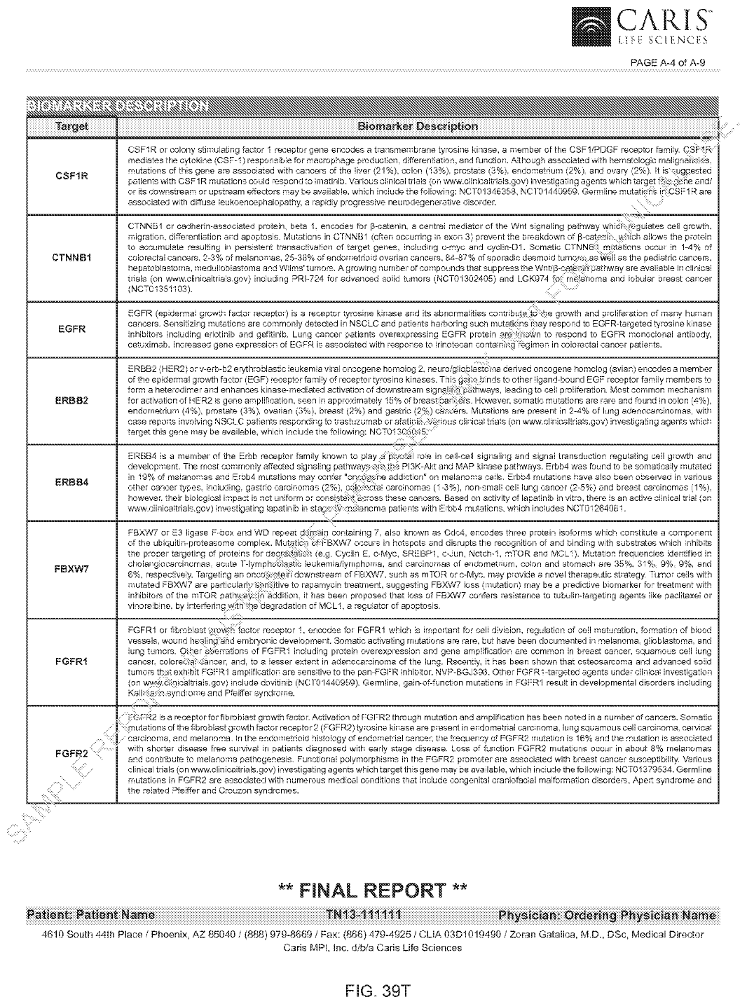

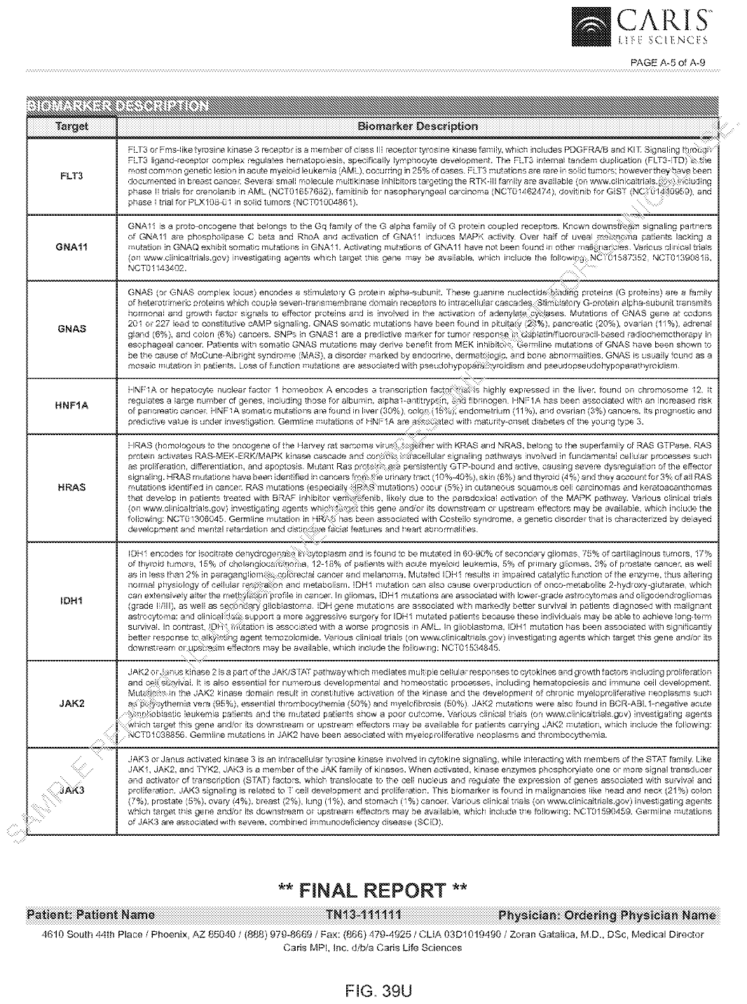

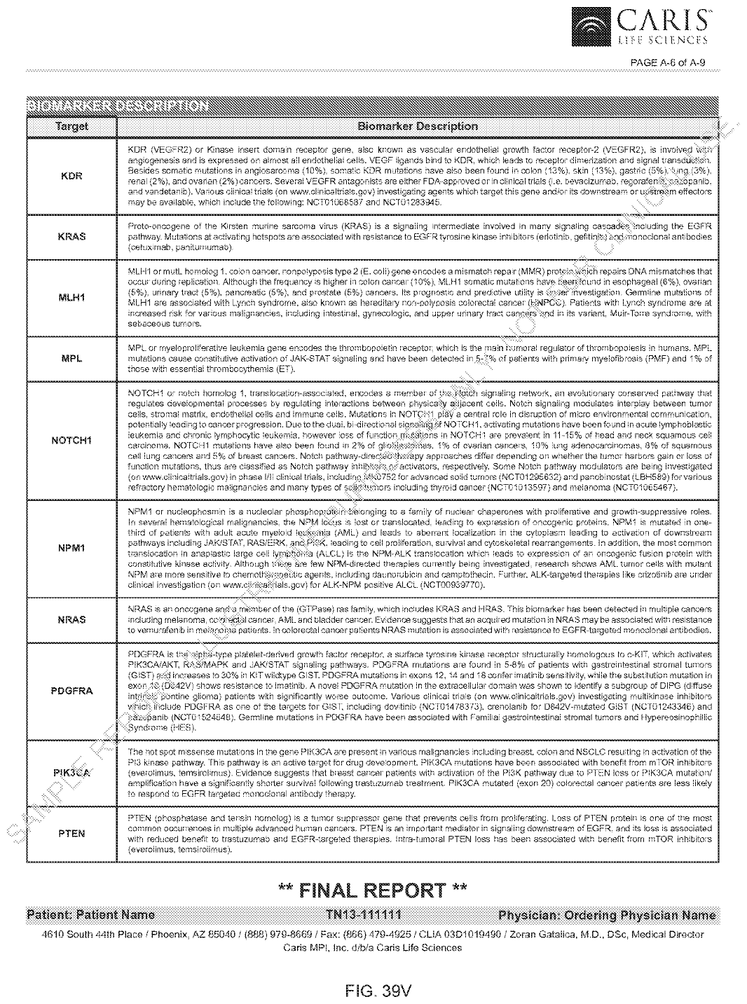

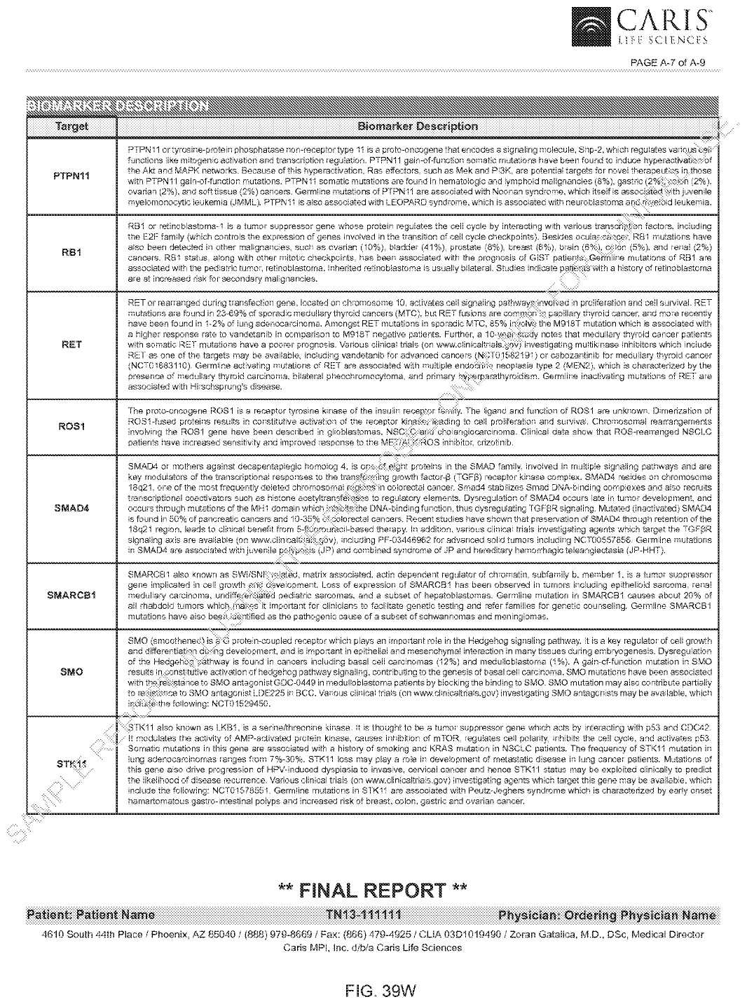

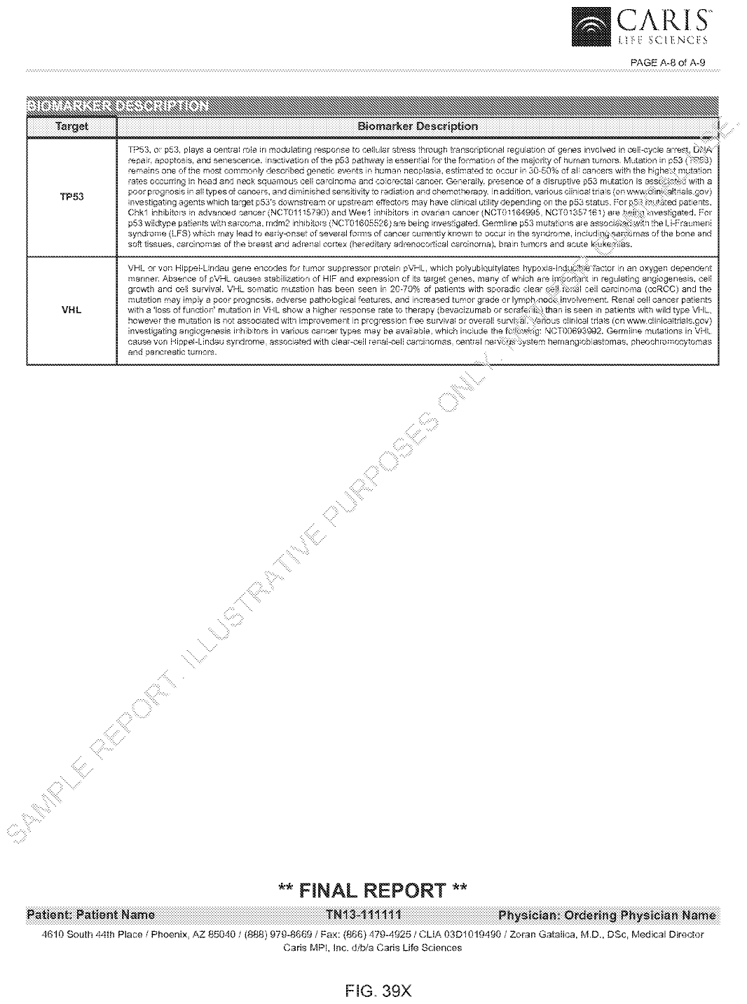

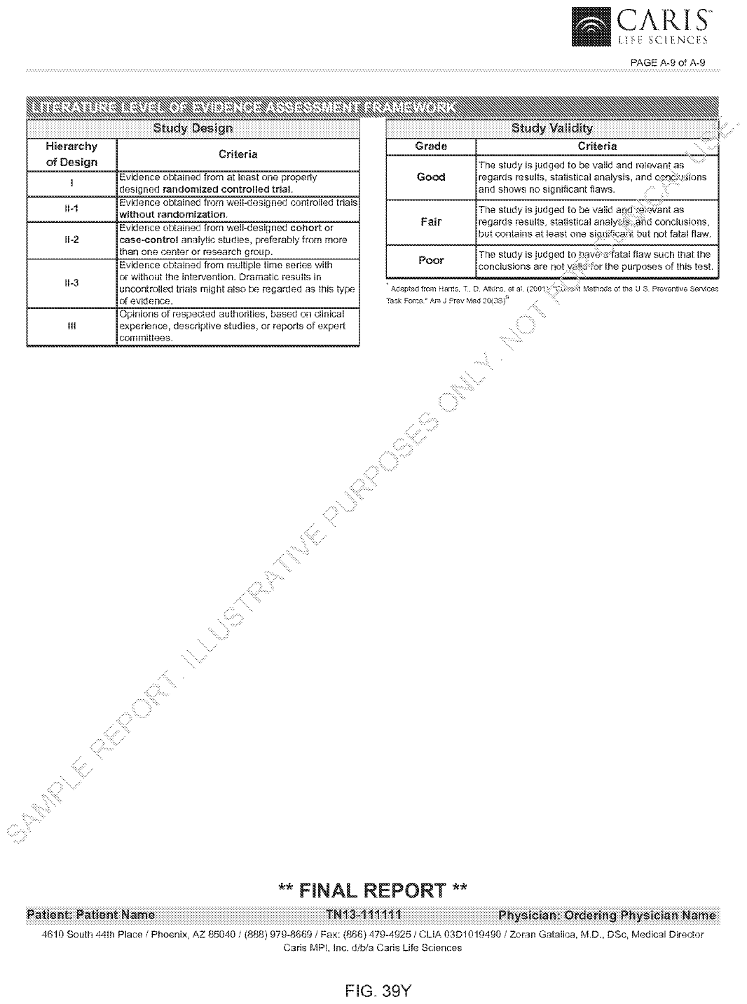

FIGS. 37-39 herein present illustrative patient reports. FIGS. 37A-Y provide an illustrative report for molecular profiling of high grade glioma (see FIGS. 33O-P and Tables 19, 21 and accompanying text) with expanded mutational analysis using Next Generation sequencing as described above (see, e.g., Tables 24-25 and accompanying text). FIG. 38 provides an illustrative report for molecular profiling of a lung adenocarcinoma (see FIGS. 33I-J and Tables 17-18 and accompanying text) with expanded mutational analysis using Next Generation sequencing as described above (see, e.g., Tables 24-25 and accompanying text). FIG. 39 provides an illustrative report for molecular profiling via mutational analysis of a non-small cell lung cancer using Next Generation sequencing as described above (see, e.g., Table 25 and accompanying text).

As noted herein, the same biomarker may be assessed by one or more technique. In such cases, the results of the different analysis may be prioritized in case of inconsistent results. For example, the different methods may detect different aspects of a single biomarker (e.g., expression level versus mutation), or one method may be more sensitive than another. In the profiles presented above in Tables 11-14, BRAF mutations for melanoma and uveal melanoma samples are assessed by both PCR and Next Generation sequencing. Results obtained using the FDA approved cobas PCR (Roche Diagnostics) may be prioritized over the Next Generation results. However, if the sequencing detects a mutation, e.g., V600E, V600E2 or V600K, when PCR either detects wild type or is not determinable, the report may contain a note describing both sets of results including any therapy that may be implicated. In the case of melanoma, when the result of BRAF cobas PCR is “Wild type” or “no data” whereas BRAF sequencing is “V600E” or “V600E2”, the report may comprise a note that BRAF mutation was not detected by the FDA-approved Cobas PCR test, however, a V600E/E2 mutation was detected by alternative methods (next generation/Sanger sequencing) and that evidence suggests that the presence of a V600E mutation associates with potential clinical benefit from vemurafenib, dabrafenib or trametinib therapy. Similarly, when the result of BRAF cobas PCR is “Wild type” or “no data” and BRAF sequencing is “V600K”, the report may comprise a note that BRAF mutation was not detected by the FDA-approved Cobas PCR test, however, a V600K mutation was detected by alternative methods (next generation/Sanger sequencing) and that evidence suggests that the presence of a V600K mutation associates with potential clinical benefit from trametinib therapy. In the case of uveal melanoma, when the result of BRAF cobas PCR is “Wild type” or “no data” and BRAF sequencing is “V600E”, or “V600E2” or “V600K”, the report may comprise a note that BRAF mutation was not detected by the FDA-approved Cobas PCR test, however, a V600E/E2 or a V600K mutation was detected by alternative methods (next generation/Sanger sequencing) and that evidence suggests that the presence of a V600E or V600K mutation associates with potential clinical benefit from vemurafenib.

### Androgen Receptor Profiling

The androgen receptor (AR) is a type of nuclear receptor that is activated by binding of either of the androgenic hormones testosterone or dihydrotestosterone in the cytoplasm and then translocating into the nucleus. AR is related to the progesterone receptor, and progestins in higher dosages can block the androgen receptor. The main function of AR is as a DNA-binding transcription factor that regulates gene expression. AR is expressed in multiple cell types and plays a role in various cancers, including without limitation prostate, bladder, kidney, lung, breast and liver. See Chang et al., Androgen receptor (AR) differential roles in hormone-related tumors including prostate, bladder, kidney, lung, breast and liver. Oncogene. 2013 Jul. 22. doi: 10.1038/onc.2013.274.

To counteract cancer cell proliferation, antiandrogenic drugs are used for hormone therapy called androgen deprivation therapy (ADT). Antiandrogens, or androgen antagonists, prevent androgens from expressing their biological effects on responsive tissues, including without limitation abarelix, bicalutamide, flutamide, gonadorelin, goserelin, leuprolide. Some antiandrogenic drugs suppress androgen production whereas others inhibit androgens from binding to the cancer cells' androgen receptors. Flutamide, nilutamide and bicalutamide are nonsteroidal antiandrogens. 5-alpha-reductase inhibitors such as finasteride, dutasteride, bexlosteride, izonsteride, turosteride, and epristeride are antiandrogenic as they prevent the conversion of testosterone to dihydrotestosterone (DHT).

Antiandrogens can be used to treat various AR expressing cancers and is most commonly associated with protstate cancer. Androgen-deprivation therapy (ADT) has been shown to cause initial reduction of prostate tumors. However, antiandrogenic treatment can cause prostate cancer tumors to become androgen independent. Androgen independence occurs when cells that are not reliant on androgen proliferate and spread while cells that require androgen for survival undergo apoptosis. The cells that do not require androgen become the basis of the tumors, causing reoccurring tumors a few years after the initial disappearance of the prostate cancer. Once prostate cancer becomes androgen independent, hormone therapy will most likely no longer benefit the individual and a new treatment approach is needed. Because the cancer can proliferate despite castrate levels of androgen, it is referred to as a castration-resistant prostate cancer (CRPC). Treatments for CRPC include the CYP17 inhibitor abiraterone, CYP17A1 inhibitors orteronel and galeterone, chemotherapeutic cabazitaxel, antiandrogens enzalutamide and ARN-509, endocrine disruptor abiraterone acetate, immunotherapy sipuleucel-T, and bone-targeting radiopharmaceutical alpharadin. See, e.g., Acar et al., New therapeutics to treat castrate-resistant prostate cancer. Scientific World Journal. 2013 May 27; 2013:379641; Mitsiades. A Road Map to Comprehensive Androgen Receptor Axis Targeting for Castration-Resistant Prostate Cancer. Cancer Res. 2013 Aug. 1; 73(15):4599-605. doi: 10.1158/0008-5472.CAN-12-4414. Enzalutamide is an androgen receptor antagonist drug developed for the treatment of metastatic castration-resistant prostate cancer. Molecular profiling according to the invention can be used to identify candidate treatments for castrate-resistant prostate cancer.

In an aspect, the invention provides a method of molecular profiling a cancer, comprising determining a level of the androgen receptor (AR) in a cancer cell. The cancer cell can be in a sample from a subject having or suspected of having the cancer. Any appropriate sample such as described herein can be used. The cancer can be treated with an antiandrogen therapy if the androgen receptor is expressed. The antiandrogen can suppress androgen production and/or inhibit androgens from binding to androgen receptors. For example, the antiandrogen can be one or more of abarelix, bicalutamide, flutamide, gonadorelin, goserelin, leuprolide, nilutamide, a 5-alpha-reductase inhibitor, finasteride, dutasteride, bexlosteride, izonsteride, turosteride, and epristeride. The cancer may be androgen independent. The expression can be assessed at the gene or gene product (e.g., protein) level. The cancer can be any appropriate type of cancer, including without limitation an acute myeloid leukemia (AML), breast carcinoma, cholangiocarcinoma, colorectal adenocarcinoma, extrahepatic bile duct adenocarcinoma, female genital tract malignancy, gastric adenocarcinoma, gastroesophageal adenocarcinoma, gastrointestinal stromal tumors (GIST), glioblastoma, head and neck squamous carcinoma, leukemia, liver hepatocellular carcinoma, low grade glioma, lung bronchioloalveolar carcinoma (BAC), lung non-small cell lung cancer (NSCLC), lung small cell cancer (SCLC), lymphoma, male genital tract malignancy, malignant solitary fibrous tumor of the pleura (MSFT), melanoma, multiple myeloma, neuroendocrine tumor, nodal diffuse large B-cell lymphoma, non epithelial ovarian cancer (non-EOC), ovarian surface epithelial carcinoma, pancreatic adenocarcinoma, pituitary carcinomas, oligodendroglioma, prostatic adenocarcinoma, retroperitoneal or peritoneal carcinoma, retroperitoneal or peritoneal sarcoma, small intestinal malignancy, soft tissue tumor, thymic carcinoma, thyroid carcinoma, or uveal melanoma. For example, the cancer can be a prostate, bladder, kidney, lung, breast, or liver cancer. In embodiments, the treatment for an AR expressing cancer comprises one or more of a CYP17 inhibitor (e.g., abiraterone), CYP17A1 inhibitor (e.g., orteronel, galeterone), chemotherapeutic agent (e.g., cabazitaxel), antiandrogen (e.g., enzalutamide, ARN-509), an endocrine disruptor (e.g., abiraterone acetate), immunotherapy (e.g., sipuleucel-T), and bone-targeting radiopharmaceutical (e.g., alpharadin).

### EXAMPLES

### Example 1: Molecular Profiling to Find Targets and Select Treatments for Refractory Cancers

The primary objective was to compare progression free survival (PFS) using a treatment regimen selected by molecular profiling with the PFS for the most recent regimen the patient progressed on (e.g. patients are their own control) (FIG. 40). The molecular profiling approach was deemed of clinical benefit for the individual patient who had a PFS ratio (PFS on molecular profiling selected therapy/PFS on prior therapy) of ≥1.3.

The study was also performed to determine the frequency with which molecular profiling by IHC, FISH and microarray yielded a target against which there is a commercially available therapeutic agent and to determine response rate (RECIST) and percent of patients without progression or death at 4 months.

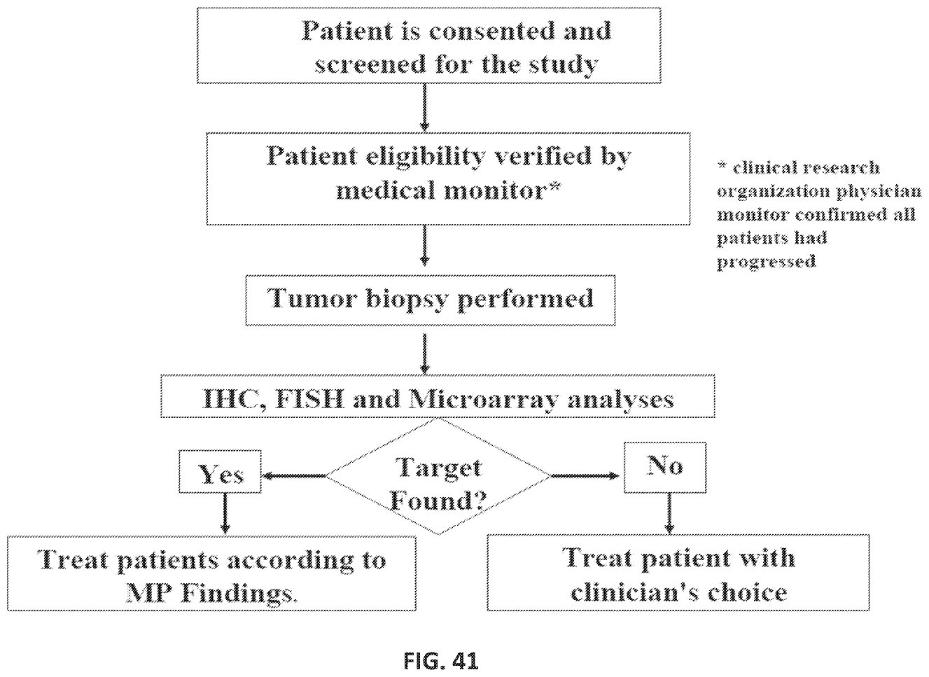

The study was conducted in 9 centers throughout the United States. An overview of the method is depicted in FIG. 41. As can be seen in FIG. 41, the patient was screened and consented for the study. Patient eligibility was verified by one of two physician monitors. The same physicians confirmed whether the patients had progressed on their prior therapy and how long that PFS (TTP) was. A tumor biopsy was then performed, as discussed below. The tumor was assayed using IHC, FISH (on paraffin-embedded material) and microarray (on fresh frozen tissue) analyses.

The results of the IHC/FISH and microarray were given to two study physicians who in general used the following algorithm in suggesting therapy to the physician caring for the patient: 1) IHC/FISH and microarray indicated same target was first priority; 2) IHC positive result alone next priority; and 3) microarray positive result alone the last priority.

The patient's physician was informed of the suggested treatment and the patient was treated with the suggested agent(s) (package insert recommendations). The patient's disease status was assessed every 8 weeks and adverse effects were assessed by the NCI CTCAE version 3.0.

To be eligible for the study, the patient was required to: 1) provide informed consent and HIPAA authorization; 2) have any histologic type of metastatic cancer; 3) have progressed by RECIST criteria on at least 2 prior regimens for advanced disease; 4) be able to undergo a biopsy or surgical procedure to obtain tumor samples; 5) be ≥18 years, have a life expectancy ≥3 months, and an Eastern Cooperative Oncology Group (ECOG) Performance Status or 0-1; 6) have measurable or evaluable disease; 7) be refractory to last line of therapy (documented disease progression under last treatment; received ≥6 weeks of last treatment; discontinued last treatment for progression); 8) have adequate organ and bone marrow function; 9) have adequate methods of birth control; and 10) if CNS metastases then adequately controlled. The ECOG performance scale is described in Oken, M. M., Creech, R. H., Tormey, D. C., Horton, J., Davis, T. E., McFadden, E. T., Carbone, P. P.: Toxicity And Response Criteria Of The Eastern Cooperative Oncology Group. Am J Clin Oncol 5:649-655, 1982, which is incorporated by reference in its entirety. Before molecular profiling was performed, the principal investigator at the site caring for the patient must designate what they would treat the patient with if no molecular profiling results were available.

Methods

All biopsies were performed at local investigators' sites. For needle biopsies, 2-3 18 gauge needle core biopsies were performed. For DNA microarray (MA) analysis, tissue was immediately frozen and shipped on dry ice via FedEx to a central CLIA certified laboratory, Caris MPI in Phoenix, Ariz. For IHC, paraffin blocks were shipped on cold packs. IHC was considered positive for target if 2+ in ≥30% of cells. The MA was considered positive for a target if the difference in expression for a gene between tumor and control organ tissue was at a significance level of p<0.001.

Ascertainment of the Time to Progression to Document the Progression-Free Survival Ratio

Time to progression under the last line of treatment was documented by imaging in 58 patients (88%). Among these 58 patients, documentation by imaging alone occurred in 49 patients (74%), and documentation by imaging with tumor markers occurred in nine patients (14%; ovarian cancer, n 3; colorectal, n 1; pancreas, n 1; prostate, n 3; breast, n 1). Patients with clinical proof of progression were accepted when the investigator reported the assessment of palpable and measurable lesions (i.e., inflammatory breast cancer, skin/subcutaneous nodules, or lymph nodes), which occurred in six patients (9%). One patient (2%) with prostate cancer was included with progression by tumor marker. In one patient (2%) with breast cancer, the progression was documented by increase of tumor marker and worsening of bone pain. The time to progression achieved with a treatment based on molecular profiling was documented by imaging in 44 patients (67%) and by clinical events detected between two scheduled tumor assessments in 20 patients. These clinical events were reported as serious adverse events related to disease progression (e.g., death, bleeding, bowel obstruction, hospitalization), and the dates of reporting were censored as progression of disease. The remaining two patients were censored at the date of last follow-up.

IHC/FISH

For IHC studies, the formalin fixed, paraffin embedded tumor samples had slices from these blocks submitted for IHC testing for the following proteins: EGFR, SPARC, C-kit, ER, PR, Androgen receptor, PGP, RRM1, TOPO1, BRCP1, MRP1, MGMT, PDGFR, DCK, ERCC1, Thymidylate synthase, Her2/neu and TOPO2A. IHCs for all proteins were not carried out on all patients' tumors.

Formalin-fixed paraffin-embedded patient tissue blocks were sectioned (4 μm thick) and mounted onto glass slides. After deparaffination and rehydration through a series of graded alcohols, pretreatment was performed as required to expose the targeted antigen.

Human epidermal growth factor receptor 2 (HER2) and epidermal growth factor receptor (EGFR) were stained as specified by the vendor (DAKO, Denmark). All other antibodies were purchased from commercial sources and visualized with a DAB biotin-free polymer detection kit. Appropriate positive control tissue was used for each antibody. Negative control slides were stained by replacing the primary antibody with an appropriately matched isotype negative control reagent. All slides were counterstained with hematoxylin as the final step and cover slipped. Tissue microarray sections were analyzed by FISH for EGFR and HER-2/neu copy number per the manufacturer's instructions. FISH for HER-2/neu (was done with the PathVysion HER2 DNA Probe Kit (Abbott Molecular, Abbott Park, Ill.). FISH for EGFR was done with the LSI EGFR/CEP 7 Probe (Abbott Molecular).

All slides were evaluated semi-quantitatively by a first pathologist, who confirmed the original diagnosis as well as read each of the immunohistochemical stains using a light microscope. Some lineage immunohistochemical stains were performed to confirm the original diagnosis, as necessary Staining intensity and extent of staining were determined; both positive, tumor-specific staining of tumor cells and highly positive (≥2+), pervasive (≥30%) tumor specific staining results were recorded. IHC was considered positive for target if staining was ≥2+ in ≥30% of cells. Rather than look for a positive signal without qualification, this approach raises the stringency of the cut point such that it would be a significant or more demonstrative positive. A higher positive is more likely to be associated with a therapy that would affect the time to progression. The cut point used (i.e., staining was ≥2+ in ≥30% of cells) is similar to some cut points used in breast cancer for HER2/neu. When IHC cut points were compared with evidence from the tissue of origin of the cancer, the cut points were equal to or higher (more stringent) than the evidence cut points. A standard 10% quality control was performed by a second pathologist.

Microarray

Tumor samples obtained for microarray were snap frozen within 30 minutes of resection and transmitted to Caris-MPI on dry ice. The frozen tumor fragments were placed on a 0.5 mL aliquot of frozen 0.5M guanidine isothiocyanate solution in a glass tube, and simultaneously thawed and homogenized with a Covaris S2 focused acoustic wave homogenizer (Covaris, Woburn, Mass.). A 0.5 mL aliquot of TriZol was added, mixed and the solution was heated to 65° C. for 5 minutes then cooled on ice and phase separated by the addition of chloroform followed by centrifugation. An equal volume of 70% ethanol was added to the aqueous phase and the mixture was chromatographed on a Qiagen RNeasy column (Qiagen, Germantown, Md.). RNA was specifically bound and then eluted. The RNA was tested for integrity by assessing the ratio of 28S to 18S ribosomal RNA on an Agilent BioAnalyzer (Agilent, Santa Clara, Calif.). Two to five micrograms of tumor RNA and two to five micrograms of RNA from a sample of a normal tissue representative of the tumor's tissue of origin were separately converted to cDNA and then labeled during T7 polymerase amplification with contrasting fluor tagged (Cy3, Cy5) cytidine triphosphate. The labeled tumor and its tissue of origin reference were hybridized to an Agilent H1Av2 60-mer olio array chip with 17,085 unique probes.

The arrays contain probes for 50 genes for which there is a possible therapeutic agent that would potentially interact with that gene (with either high expression or low expression). Those 50 genes included: ADA, AR, ASNA, BCL2, BRCA2, CD33, CDW52, CES2, DNMT1, EGFR, ERBB2, ERCC3, ESR1, FOLR2, GART, GSTP1, HDAC1, HIF1A, HSPCA, IL2RA, KIT, MLH1, MS4A1, MASH2, NFKB2, NFKBIA, OGFR, PDGFC, PDGFRA, PDGFRB, PGR, POLA, PTEN, PTGS2, RAF1, RARA, RXRB, SPARC, SSTR1, TK1, TNF, TOP1, TOP2A, TOP2B, TXNRD1, TYMS, VDR, VEGF, VHL, and ZAP70.

The chips were hybridized from 16 to 18 hours at 60° C. and then washed to remove non-stringently hybridized probe and scanned on an Agilent Microarray Scanner. Fluorescent intensity data were extracted, normalized, and analyzed using Agilent Feature Extraction Software. Gene expression was judged to be different from its reference based on an estimate of the significance of the extent of change, which was estimated using an error model that takes into account the levels of signal to noise for each channel, and uses a large number of positive and negative controls replicated on the chip to condition the estimate. Expression changes at the level of p≤0.001 were considered as significantly different.

Statistical Considerations

The protocol called for a planned 92 patients to be enrolled of which an estimated 64 patients would be treated with therapy assigned by molecular profiling. The other 28 patients were projected to not have molecular profiling results available because of (a) inability to biopsy the patient; (b) no target identified by the molecular profiling; or (c) deteriorating performance status. Sixty four patients were required to receive molecular profiling treatment in order to reject the null hypothesis (Ho) that: ≤15% of patients would have a PFS ratio of ≥1.3 (e.g. a non-promising outcome).

Treatment Selection

Treatment for the patients based on molecular profiling results was selected using the following algorithm: 1) IHC/FISH and microarray indicates same target; 2) IHC positive result alone; 3) microarray positive result alone. The patient's physician was informed of suggested treatment and the patient was treated based on package insert recommendations. Disease status was assessed every 8 weeks. Adverse effects were assessed by NCI CTCAE version 3.0.

The targets and associated drugs are listed in Table 30.

TABLE 30
Pairings of Targets and Drugs

| Potential Target | Agents Suggested as Interacting With the Target |
|---|---|
| IHC | |
| EGFR | Cetuximab, erlotinib, gefitinib |
| SPARC | Nanoparticle albumin-bound paclitaxel |
| c-KIT | Imatinib, sunitinib, sorafenib |
| ER | Tamoxifen, aromatase inhibitors, toremifene, |
| | progestational agent |
| PR | Progestational agents, tamoxifen, aromatase |
| | inhibitor, goserelin |
| Androgen receptor | Flutamide, abarelix, bicalutamide, leuprolide, |
| | goserelin |
| PGP | Avoid natural products, doxorubicin, etoposide, |
| | docetaxel, vinorelbine |
| HER2/NEU | Trastuzumab |
| PDGFR | Sunitinib, imatinib, sorafenib |
| CD52 | Alemtuzumab |
| CD25 | Denileukin diftitox |
| HSP90 | Geldanamycin, CNF2024 |
| TOP2A | Doxorubicin, epirubicin, etoposide |
| Microarray | |
| ADA | Pentostatin, cytarabine |
| AR | Flutamide, abarelix, bicalutamide, leuprolide, |
| | goserelin |
| ASNA | Asparaginase |
| BCL2 | Oblimersen sodium† |
| BRCA2 | Mitomycin |
| CD33 | Gemtuzumab ozogamicin |
| CDW52 | Alemtuzumab |
| CES-2 | Irinotecan |
| DCK | Gemcitabine |
| DNMT1 | Azacitidine, decitabine |
| EGFR | Cetuximab, erlotinib, gefitinib |
| ERBB2 | Trastuzumab |
| ERCC1 | Cisplatin, carboplatin, oxaliplatin |
| ESR1 | Tamoxifen, aromatase inhibitors, toremifene, |
| | progestational agent |
| FOLR2 | Methotrexate, pemetrexed |
| GART | Pemetrexed |
| GSTP1 | Platinum |
| HDAC1 | Vorinostat |
| HIF1α | Bevacizumab, sunitinib, sorafenib |
| HSPCA | Geldanamycin, CNF2024 |
| IL2RA | Aldesleukin |
| KIT | Imatinib, sunitinib, sorafenib |
| MLH-1 | Gemcitabine, oxaliplatin |
| MSH1 | Gemcitabine |
| MSH2 | Gemcitabine, oxaliplatin |
| NFKB2 | Bortezomib |
| NFKB1 | Bortezomib |
| OGFR | Opioid growth factor |
| PDGFC | Sunitinib, imatinib, sorafenib |
| PDGFRA | Sunitinib, imatinib, sorafenib |
| PDGFRB | Sunitinib, imatinib, sorafenib |
| PGR | Progestational agents, tamoxifen, aromatase |
| | inhibitors, goserelin |
| POLA | Cytarabine |
| PTEN | Rapamycin (if low) |
| PTGS2 | Celecoxib |
| RAF1 | Sorafenib |
| RARA | Bexarotene, all-trans-retinoic acid |
| RXRB | Bexarotene |
| SPARC | Nanoparticle albumin-bound paclitaxel |
| SSTR1 | Octreotide |
| TK1 | Capecitabine |
| TNF | Infliximab |
| TOP1 | Irinotecan, topotecan |
| TOP2A | Doxorubicin, etoposide, mitoxantrone |
| TOP2B | Doxorubicin, etoposide, mitoxantrone |
| TXNRD1 | Px12 |
| TYMS | Fluorouracil, capecitabine |
| VDR | Calcitriol |
| VEGF | Bevacizumab, sunitinib, sorafenib |
| VHL | Bevacizumab, sunitinib, sorafenib |
| ZAP70 | Geldanamycin, CNF2024 |

Results

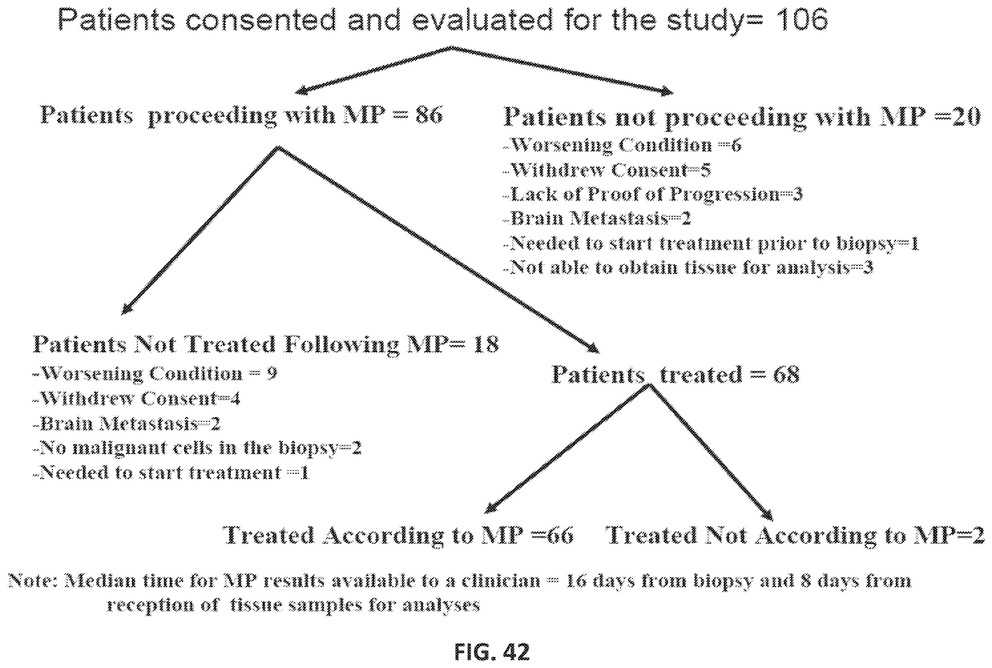

The distribution of the patients is diagrammed in FIG. 42 and the characteristics of the patients shown in Tables 31 and 32. As can be seen in FIG. 42, 106 patients were consented and evaluated. There were 20 patients who did not proceed with molecular profiling for the reasons outlined in FIG. 42 (mainly worsening condition or withdrawing their consent or they did not want any additional therapy). There were 18 patients who were not treated following molecular profiling (mainly due to worsening condition or withdrawing consent because they did not want additional therapy). There were 68 patients treated, with 66 of them treated according to molecular profiling results and 2 not treated according to molecular profiling results. One of the two was treated with another agent because the clinician caring for the patient felt a sense of urgency to treat and the other was treated with another agent because the insurance company would not cover the molecular profiling suggested treatment.

The median time for molecular profiling results being made accessible to a clinician was 16 days from biopsy (range 8 to 30 days) and a median of 8 days (range 0 to 23 days) from receipt of the tissue sample for analysis. Some modest delays were caused by the local teams not sending the patients' blocks immediately (due to their need for a pathology workup of the specimen). Patient tumors were sent from 9 sites throughout the United States including: Greenville, S.C.; Tyler, Tex.; Beverly Hills, Calif.; Huntsville, Ala.; Indianapolis, Ind.; San Antonio, Tex.; Scottsdale, Ariz. and Los Angeles, Calif.

Table 31 details the characteristics of the 66 patients who had molecular profiling performed on their tumors and who had treatment according to the molecular profiling results. As seen in Table 32, of the 66 patients the majority were female, with a median age of 60 (range 27-75). The number of prior treatment regimens was 2-4 in 53% of patients and 5-13 in 38% of patients. There were 6 patients (9%), who had only 1 prior therapy because no approved active 2^{nd} line therapy was available. Twenty patients had progressed on prior phase I therapies. The majority of patients had an ECOG performance status of 1.

TABLE 31
Patient Characteristics (n = 66)

| Characteristic | n | % |
|---|---|---|
| Gender | | |
| Female | 43 | 65 |
| Male | 23 | 35 |
| Age | | |
| Median (range) | 60 | (27-75) |
| Number of Prior Treatments | | |
| 2-4* | 35 | 53 |
| 5-13 | 25 | 38 |
| ECOG | | |
| 0 | 18 | 27 |
| 1 | 48 | 73 |

*Note:
6 patients (9%) had 1 prior

As seen in Table 32, tumor types in the 66 patients included breast cancer 18 (27%), colorectal 11 (17%), ovarian 5 (8%), and 32 patients (48%) were in the miscellaneous categories. Many patients had the more rare types of cancers.

TABLE 32
Patient Tumor Types (n = 66)

| Tumor Type | n | % |
|---|---|---|
| Breast | 18 | 27 |
| Colorectal | 11 | 17 |
| Ovarian | 5 | 8 |
| Miscellaneous | 32 | 48 |
| Prostate | 4 | 6 |
| Lung | 3 | 5 |
| Melanoma | 2 | 3 |
| Small cell (esopha/retroperit) | 2 | 3 |
| Cholangiocarcinoma | 2 | 3 |
| Mesothelioma | 2 | 3 |
| H&N (SCC) | 2 | 3 |
| Pancreas | 2 | 3 |
| Pancreas neuroendocrine | 1 | 1.5 |
| Unknown (SCC) | 1 | 1.5 |
| Gastric | 1 | 1.5 |
| Peritoneal pseudomyxoma | 1 | 1.5 |
| Anal Canal (SCC) | 1 | 1.5 |
| Vagina (SCC) | 1 | 1.5 |
| Cervis | 1 | 1.5 |
| Renal | 1 | 1.5 |
| Eccrine seat adenocarinoma | 1 | 1.5 |
| Salivary gland adenocarinoma | 1 | 1.5 |
| Soft tissue sarcoma (uterine) | 1 | 1.5 |
| GIST (Gastric) | 1 | 1.5 |
| Thyroid-Anaplastic | 1 | 1.5 |

Primary Endpoint: PFS Ratio ≥1.3

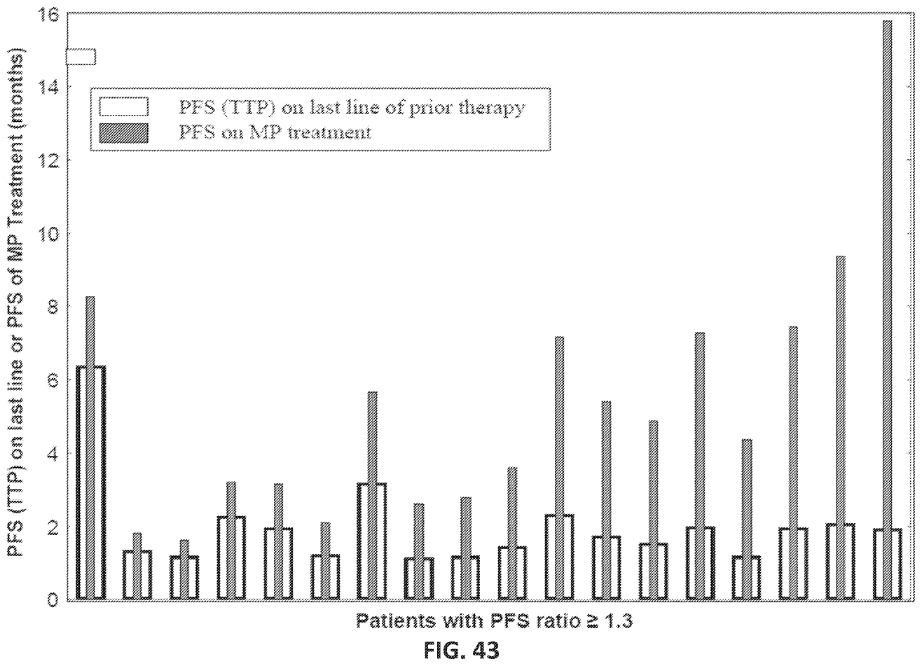

As far as the primary endpoint for the study is concerned (PFS ratio of ≥1.3), in the 66 patients treated according to molecular profiling results, the number of patients with PFS ratio greater or equal to 1.3 was 18 out of the 66 or 27%, 95% CI 17-38% one-sided, one-sample non parametric test p=0.007. The null hypothesis was that <15% of this patient population would have a PFS ratio of ≥1.3. Therefore, the null hypothesis is rejected and our conclusion is that this molecular profiling approach is beneficial. FIG. 43 details the comparison of PFS on molecular profiling therapy (the bar) versus PFS (TTP) on the patient's last prior therapy (the boxes) for the 18 patients. The median PFS ratio is 2.9 (range 1.3-8.15).

If the primary endpoint is examined, as shown in Table 33, a PFS ratio ≥1.3 was achieved in 8/18 (44%) of patients with breast cancer, 4/11 (36%) patients with colorectal cancer, 1/5 (20%) of patients with ovarian cancer and 5/32 (16%) patients in the miscellaneous tumor types (note that miscellaneous tumor types with PFS ratio ≥1.3 included: lung 1/3, cholangiocarcinoma 1/3, mesothelioma 1/2, eccrine sweat gland tumor 1/1, and GIST (gastric) 1/1).

TABLE 33
Primary Endpoint - PFS Ratio ≥ 1.3 By Tumor Type

| | | Number with PFS | |
|---|---|---|---|
| Tumor Type | Total Treated | Ratio ≥ 1.3 | % |
| Breast | 18 | 8 | 44 |
| Colorectal | 11 | 4 | 36 |
| Ovarian | 5 | 1 | 20 |
| Miscellaneous* | 32 | 5 | 16 |
| Total | 66 | 18 | 27 |

*lung ⅓, cholangiocarcinoma ½, mesothelioma ½, eccrine sweat 1/1, GIST (gastric) 1/1

The treatment that the 18 patients with the PFS≥1.3 received based on profiling is detailed in Table 34. As can be seen in that table for breast cancer patients, the treatment ranged from diethylstilbestrol to nab paclitaxel+gemcitabine to doxorubicin. Treatments for patients with other tumor types are also detailed in Table 34. The table further shows a comparison of the drugs that the responding patients received versus the drugs that would have been suggested without molecular profiling and indicates which targets were used to suggest the therapies. Overall, 14 were treated with combinations and 4 were treated with single agents.

TABLE 34
Targets Noted in Patients' Tumors, Treatment Suggested on
the Basis of These Results, and Treatment Investigator Would
Use if No Target Was Identified (in patients with PFS ratio ≥ 1.3)

| | | | Treatment the |
|---|---|---|---|
| | | Treatment Suggested | Investigator Would |
| Location of | Targets Used to | on Basis of Patient's | Have Used if No |
| Primary | Suggest Treatment | Tumor Molecular | Results From |
| Tumor | and Method Used | Profiling | Molecular Profiling |
| Breast | ESR1: I; ESR1: M | DES 5 mg TID | Investigational |
| Cholangiocarcinoma | EGFR: I; TOP1: M | CPT-11 350 mg/m² | Investigational |
| | | every 3 weeks; | |
| | | cetuximab 400 mg/m² | |
| | | day 1, 250 mg/m² every | |
| | | week | |
| Breast | SPARC: I; SPARC, | NAB paclitaxel 260 | Docetaxel, |
| | ERBB2: M | mg/m² every 3 weeks; | trastuzumab |
| | | trastuzumab 6 mg/kg | |
| | | every 3 weeks | |
| Eccrine sweat gland | c-KIT: I; c-KIT: M | Sunitinib 50 mg/d, 4 | Best supportive |
| (right forearm) | | weeks on/2 weeks off | care |
| Ovary | HER2/NEU, ER: I; | Lapatinib 1,250 mg PO | Bevacizumab |
| | HER2/NEU: M | days 1-21; tamoxifen 20 | |
| | | mg PO | |
| Colon/rectum | PDGFR, c-KIT: I I; | CPT-11 70 mg/m² | Cetuximab |
| | PDGFR, TOP1: M | weekly for 4 weeks on/2 | |
| | | weeks off; sorafenib | |
| | | 400 mg BID | |
| Breast | SPARC: I; DCK: M | NAB paclitaxel 90 | Mitomycin |
| | | mg/m² every 3 weeks; | |
| | | gemcitabine 750 mg/m² | |
| | | days 1, 8, 15, every 3 | |
| | | weeks | |
| Breast | ER: I; ER, TYMS: M | Letrozole 2.5 mg daily; | Capecitabine |
| | | capecitabine 1,250 | |
| | | mg/m² BID, 2 weeks | |
| | | on/1 week off | |
| Malignant | MLH1, MLH2: I; | Gemcitabine 1,000 | Gemcitabine |
| mesothelioma | RRM2B, RRM1, RRM2, | mg/m² days 1 and 8, | |
| | TOP2B: M | every 3 weeks; | |
| | | etoposide 50 mg/m² 3 | |
| | | days every 3 weeks | |
| Breast | MSH2 | Oxaliplatin 85 mg/m | Investigational |
| | | every 2 weeks; | |
| | | fluorouracil (5FU) | |
| | | 1,200 mg/m² days 1 and | |
| | | 2, every 2 weeks; | |
| | | trastuzumab 4 mg/kg | |
| | | day 1, 2 mg/kg every | |
| | | week | |
| Non-small-cell | EGFR: I; EGFR | Cetuximab 400 mg/m² | Vinorelbine |
| lung cancer | | day 1, 250 mg/m² every | |
| | | week; CPT-11 125 | |
| | | mg/m² weekly for 4 | |
| | | weeks on/2 weeks off | |
| Colon/rectum | MGMT | Temozolomide 150 | Capecitabine |
| | | mg/m² for 5 days every | |
| | | 4 weeks; bevacizumab 5 | |
| | | mg/kg every 2 weeks | |
| Colon/rectum | PDGFR, c-KIT: I; | Mitomycin 10 mg once | Capecitabine |
| | PDGFR: KDR, HIF1A, | every 4-6 weeks; | |
| | BRCA2: M | sunitinib 37.5 mg/d, 4 | |
| | | weeks on/2 weeks off | |
| Breast | DCK, DHFR: M | Gemcitabine 1,000 | Best supportive |
| | | mg/m² days 1 and 8 | care |
| | | every 3 weeks; | |
| | | pemetrexed 500 mg/m² | |
| | | days 1 and 8, every 3 | |
| | | weeks | |
| Breast | TOP2A: I; TOP2A: M | Doxorubicin 50 mg/m² | Vinorelbine |
| | | every 3 weeks | |
| Colon/rectum | MGMT, VEGFA, | Temozolomide 150 | Panitumumab |
| | HIF1A: M | mg/m² for 5 days every | |
| | | 4 weeks; sorafenib 400 | |
| | | mg BID | |
| Breast | ESR1, PR: I; ESR1, PR: | Exemestane 25 mg | Doxorubicin |
| | M | every day | liposomal |
| GIST (stomach) | EGFR: I; EGFR, | Gemcitabine 1,000 | None |
| | RRM2: M | mg/m² days 1, 8, and 15 | |
| | | every 4 weeks; | |
| | | cetuximab 400 mg/m² | |
| | | day 1, 250 mg/m2 every | |
| | | week | |

*Abbreviations used in Table 35: I, immunohistochemistry; M, microarray: DES, diethylstilbestrol; CPT-11, irinotecan; TID, three times a day; NAB, nanoparticle albumin bound; PO, orally; BID, twice a day; GIST, GI stromal tumor.

Secondary Endpoints

The results for the secondary endpoint for this study are as follows. The frequency with which molecular profiling of a patients' tumor yielded a target in the 86 patients where molecular profiling was attempted was 84/86 (98%). Broken down by methodology, 83/86 (97%) yielded a target by IHC/FISH and 81/86 (94%) yielding a target by microarray. RNA was tested for integrity by assessing the ratio of 28S to 18S ribosomal RNA on an Agilent BioAnalyzer. 83/86 (97%) specimens had ratios of 1 or greater and gave high intra-chip reproducibility ratios. This demonstrates that very good collection and shipment of patients' specimens throughout the United States and excellent technical results can be obtained.

By RECIST criteria in 66 patients, there was 1 complete response and 5 partial responses for an overall response rate of 10% (one CR in a patient with breast cancer and PRs in breast, ovarian, colorectal and NSCL cancer patients). Patients without progression at 4 months included 14 out of 66 or %.

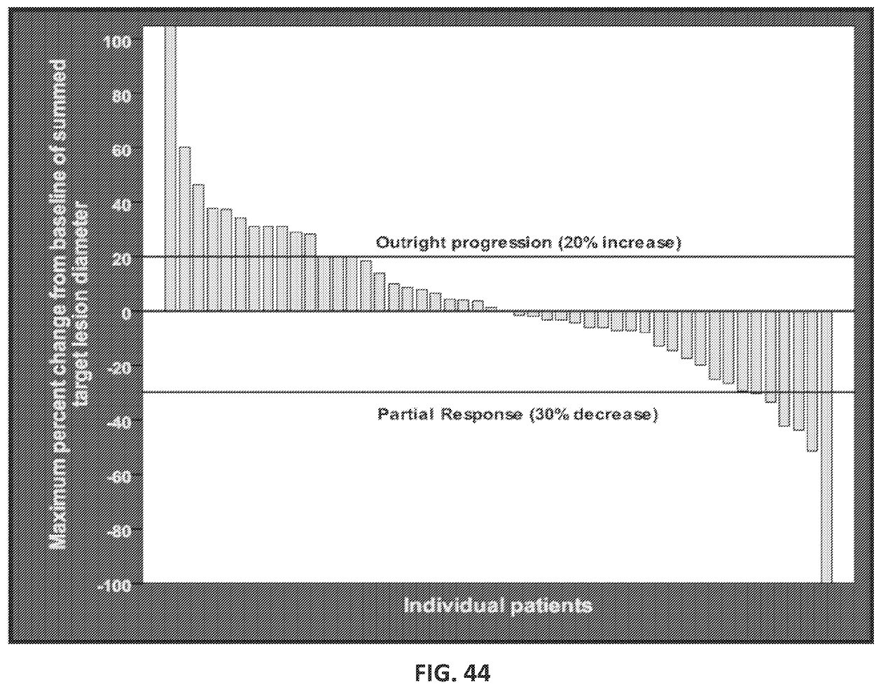

In an exploratory analysis, a waterfall plot for all patients for maximum % change of the summed diameters of target lesions with respect to baseline diameters was generated. The patients who had progression and the patients who had some shrinkage of their tumor sometime during their course along with those partial responses by RECIST criteria is demonstrated in FIG. 44. There is some shrinkage of patient's tumors in over 47% of the patients (where 2 or more evaluations were completed).

Other Analyses—Safety

As far as safety analyses there were no treatment related deaths. There were nine treatment related serious adverse events including anemia (2 patients), neutropenia (2 patients), dehydration (1 patient), pancreatitis (1 patient), nausea (1 patient), vomiting (1 patient), and febrile neutropenia (1 patient). Only one patient (1.5%) was discontinued due to a treatment related adverse event of grade 2 fatigue.

Other Analyses—Relationship Between What the Clinician Caring for the Patient would have Selected Versus What the Molecular Profiling Selected

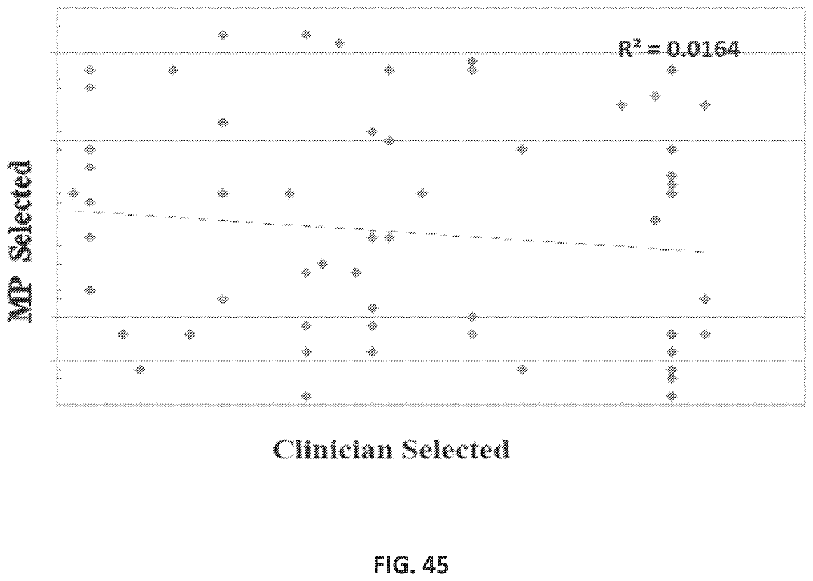

The relationship between what the clinician selected to treat the patient before knowing what molecular profiling results suggested for treatment was also examined. As detailed in FIG. 45, there is no pattern between the two. More specifically, no matches for the 18 patients with PFS ratio ≥1.3 were noted.

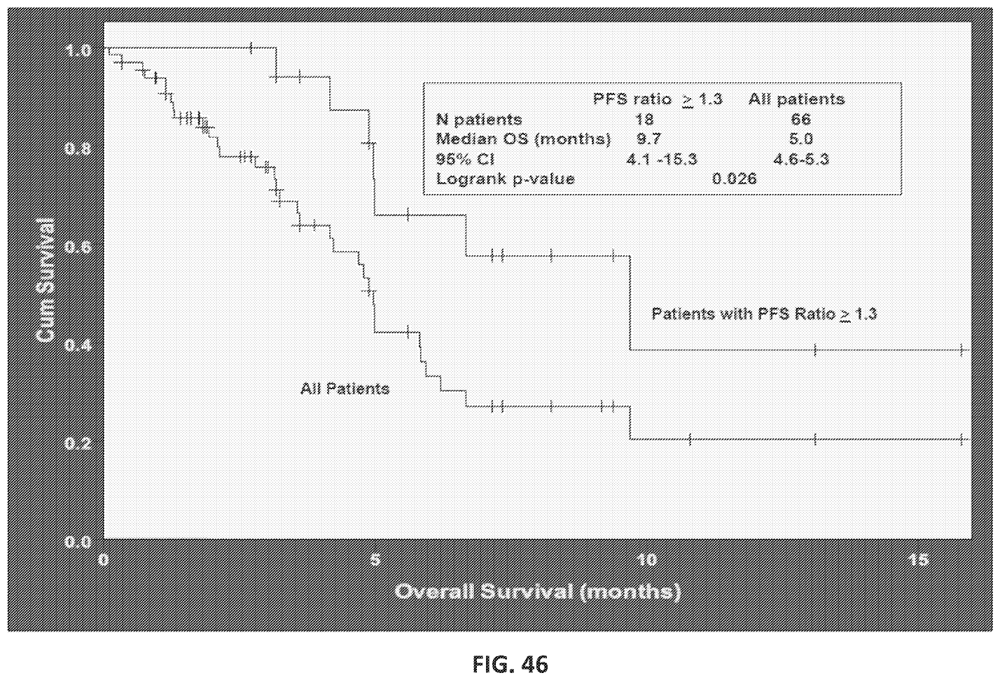

The overall survival for the 18 patients with a PFS ratio of ≥1.3 versus all 66 patients is shown in FIG. 46. This exploratory analysis was done to help determine if the PFS ratio had some clinical relevance. The overall survival for the 18 patients with the PFS ratio of ≥1.3 is 9.7 months versus 5 months for the whole population—log rank 0.026. This exploratory analysis indicates that the PFS ratio is correlated with the clinical parameter of survival.

### Conclusions

This prospective multi-center pilot study demonstrates: (a) the feasibility of measuring molecular targets in patients' tumors from 9 different centers across the US with good quality and sufficient tumor collection—and treat patients based on those results; (b) this molecular profiling approach gave a longer PFS for patients on a molecular profiling suggested regimen than on the regimen they had just progressed on for 27% of the patients (confidence interval 17-38%) p=0.007; and (c) this is a promising result demonstrating use and benefits of molecular profiling.

The results also demonstrate that patients with refractory cancer can commonly have simple targets (such as ER) for which therapies are available and can be beneficial to them. Molecular profiling for patients who have exhausted other therapies and who are perhaps candidates for phase I or II trials could have this molecular profiling performed.

### Example 2: Molecular Profiling System

Molecular profiling is performed to determine a treatment for a disease, typically a cancer. Using a molecular profiling approach, molecular characteristics of the disease itself are assessed to determine a candidate treatment. Thus, this approach provides the ability to select treatments without regard to the anatomical origin of the diseased tissue, or other “one-size-fits-all” approaches that do not take into account personalized characteristics of a particular patient's affliction. The profiling comprises determining gene and gene product expression levels, gene copy number and mutation analysis. Treatments are identified that are indicated to be effective against diseased cells that overexpress certain genes or gene products, underexpress certain genes or gene products, carry certain chromosomal aberrations or mutations in certain genes, or any other measureable cellular alterations as compared to non-diseased cells. Because molecular profiling is not limited to choosing amongst therapeutics intended to treat specific diseases, the system has the power to take advantage of any useful technique to measure any biological characteristic that can be linked to a therapeutic efficacy. The end result allows caregivers to expand the range of therapies available to treat patients, thereby providing the potential for longer life span and/or quality of life than traditional “one-size-fits-all” approaches to selecting treatment regimens.

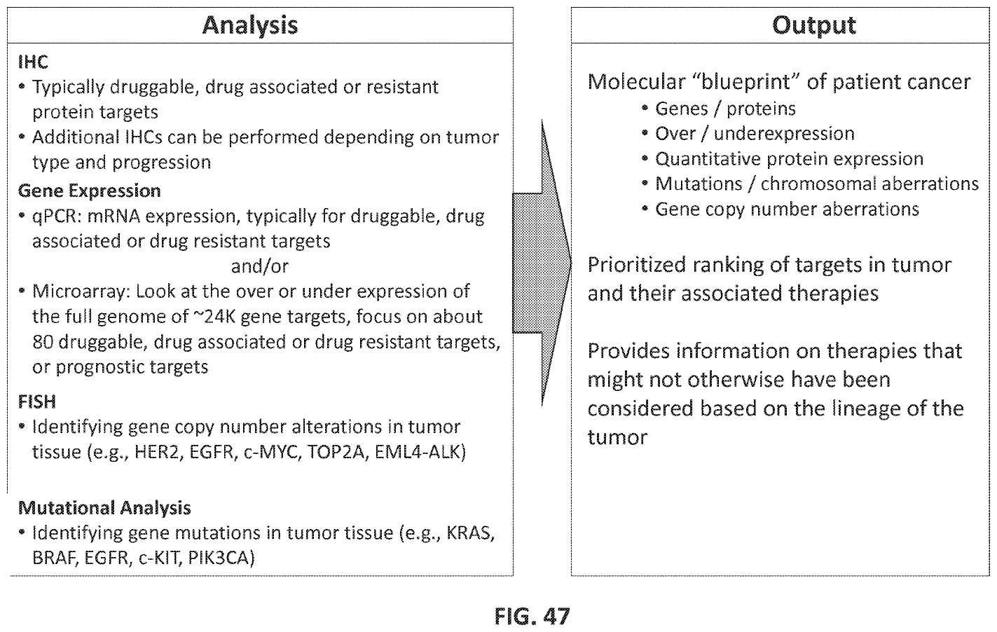

A molecular profiling system has several individual components to measure expression levels, chromosomal aberrations and mutations. The components are shown in FIG. 47. The input sample can be formalin fixed paraffin embedded (FFPE) cancer tissue.

Gene expression analysis is performed using an expression microarray or qPCR (RT-PCR). The qPCR can be performed using a low density microarray. In addition to gene expression analysis, the system can perform a set of immunohistochemistry assays on the input sample. Gene copy number is determined for a number of genes via FISH (fluorescence in situ hybridization) and mutation analysis is done by DNA sequencing (including sequence sensitive PCR assays and fragment analysis such as RFLP, as desired) for a several specific mutations. All of this data is stored for each patient case. Data is reported from the expression, IHC, FISH and DNA sequencing analysis. All laboratory experiments are performed according to Standard Operating Procedures (SOPs).

Expression can be measured using real-time PCR (qPCR, RT-PCR). The analysis can employ a low density microarray. The low density microarray can be a PCR-based microarray, such as a Taqman™ Low Density Microarray (Applied Biosystems, Foster City, Calif.).

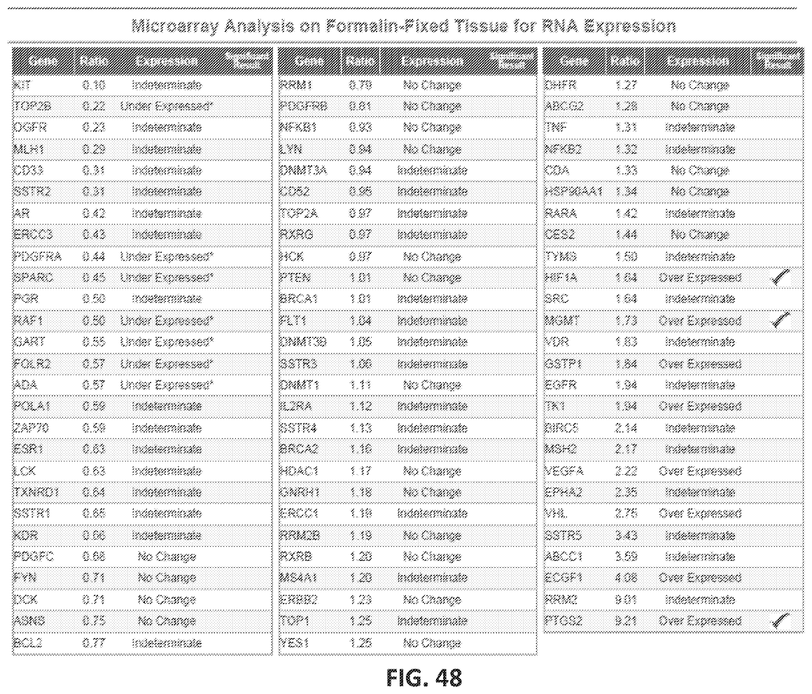

Expression can be measured using a microarray. The expression microarray can be an Agilent 44K chip (Agilent Technologies, Inc., Santa Clara, Calif.). This system is capable of determining the relative expression level of roughly 44,000 different sequences through RT-PCR from RNA extracted from fresh frozen tissue. Alternately, the system uses the Illumina Whole Genome DASL assay (Illumina Inc., San Diego, Calif.), which offers a method to simultaneously profile over 24,000 transcripts from minimal RNA input, from both fresh frozen (FF) and formalin-fixed paraffin embedded (FFPE) tissue sources, in a high throughput fashion. The analysis makes use of the Whole-Genome DASL Assay with UDG (Illumina, cat # DA-903-1024/DA-903-1096), the Illumina Hybridization Oven, and the Illumina iScan System according to the manufacturer's protocols. FIG. 48 shows results obtained from microarray profiling of an FFPE sample. Total RNA was extracted from tumor tissue and was converted to cDNA. The cDNA sample was then subjected to a whole genome (24K) microarray analysis using the Illumina Whole Genome DASL process. The expression of a subset of 80 genes was then compared to a tissue specific normal control and the relative expression ratios of these 80 target genes indicated in the figure was determined as well as the statistical significance of the differential expression.

DNA for mutation analysis is extracted from formalin-fixed paraffin-embedded (FFPE) tissues after macrodissection of the fixed slides in an area that % tumor nuclei ≥10% as determined by a pathologist. Extracted DNA is only used for mutation analysis if % tumor nuclei ≥10%. DNA is extracted using the QIAamp DNA FFPE Tissue kit according to the manufacturer's instructions (QIAGEN Inc., Valencia, Calif.). DNA can also be extracted using the QuickExtract™ FFPE DNA Extraction Kit according to the manufacturer's instructions (Epicentre Biotechnologies, Madison, Wis.). The BRAF Mutector I BRAF Kit (TrimGen, cat # MH1001-04) is used to detect BRAF mutations (TrimGen Corporation, Sparks, Md.). The DxS KRAS Mutation Test Kit (DxS, # KR-03) is used to detect KRAS mutations (QIAGEN Inc., Valencia, Calif.). BRAF and KRAS sequencing of amplified DNA is performed using Applied Biosystems' BigDye® Terminator V1.1 chemistry (Life Technologies Corporation, Carlsbad, Calif.).

IHC is performed according to standard protocols. IHC detection systems vary by marker and include Dako's Autostainer Plus (Dako North America, Inc., Carpinteria, Calif.), Ventana Medical Systems Benchmark® XT (Ventana Medical Systems, Tucson, Ariz.), and the Leica/Vision Biosystems Bond System (Leica Microsystems Inc., Bannockburn, Ill.). All systems are operated according to the manufacturers' instructions. American Society of Clinical Oncology (ASCO) and College of American Pathologist (CAP) standards are followed for ER, PR, and HER2 testing. ER, PR and HER2 as well as Ki-67, p53, and E-cad IHCs analyzed by the ACIS® (Automated Cellular Imaging System). The ACIS system comprises a microscope that scans the slides and constructs an image of the entire tissue section. Ten areas of tumor are analyzed for percentage positive cells and staining intensity within the selected fields.

FISH is performed on formalin-fixed paraffin-embedded (FFPE) tissue. FFPE tissue slides for FISH must be Hematoxylin and Eosin (H&E) stained and given to a pathologist for evaluation. Pathologists will mark areas of tumor for FISH analysis. The pathologist report shows whether tumor is present and sufficient enough to perform a complete analysis. FISH is performed using the Abbott Molecular VP2000 according to the manufacturer's instructions (Abbott Laboratories, Des Plaines, Iowa).

Illustrative reports generated by the system are shown in International PCT Patent Application PCT/US2010/000407, filed Feb. 11, 2010; and International PCT Patent Application PCT/US2010/54366, filed Oct. 27, 2010, each of which application is incorporated herein by reference in its entirety.

### Example 3: Workflow for Identifying a Therapeutic Agent

FIG. 49 illustrates a diagram that outlines a workflow for identifying a therapeutic agent by analyzing a sample from an individual with breast cancer (441). The sample is cut into a number of slides (442) and stained with hematoxylin and eosin (H&E) (443). The stained slides are read by a pathologist (444) to determine what panel of markers to test, e.g., whether to analyze the sample using a complete biomarker panel analysis or a tumor-specific biomarker panel analysis, e.g., for breast cancer sample analysis (445). The pathologist also identifies sections (446) for DNA microarray analysis (447), FISH analysis, e.g., to measure HER2 expression (448), or mutational analysis via sequencing (449). DNA microarray analysis can be performed on a whole genome scale, with focus on genes that are informative for therapeutic treatment options, including at least ABCC1, ABCG2, ADA, AR, ASNS, BCL2, BIRC5, BRCA1, BRCA2, CD33, CD52, CDA, CES2, DCK, DHFR, DNMT1, DNMT3A, DNMT3B, ECGF1, EGFR, EPHA2, ERBB2, ERCC1, ERCC3, ESR1, FLT1, FOLR2, FYN, CART, GNRH1, GSTP1, HCK, HDAC1, HIF1A, HSP90AA1, IGFBP3, IGFBP4, IGFBP5, IL2RA, KDR, KIT, LCK, LYN, MET, MGMT, MLH1, MS4A1, MSH2, NFKB1, NFKB2, NFKB1A, OGFR, PARP1, PDGFC, PDGFRA, PDGFRB, PGP, PGR, POLA1, PTEN, PTGS2, PTPN12, RAF1, RARA, RRM1, RRM2, RRM2B, RXRB, RXRG, SIK2, SPARC, SRC, SSTR1, SSTR2, SSTR3, SSTR4, SSTR5, TK1, TNF, TOP1, TOP2A, TOP2B, TXNRD1, TYMS, VDR, VEGFA, VHL, YES1, and ZAP70. IHC is run on selected sections to analyze expression of biomarkers including AR, c-kit, CAV-1, CK 5/6, CK14, CK17, ECAD, ER, Her2/Neu, Ki67, MRP1, P53, PDGFR, PGP, PR, PTEN, SPARC, TLE3 and TS (4410). Each marker can be analyzed using a single or multiple antibodies for IHC detection. For example, SPARC is detected using an anti-SPARC monoclonal antibody (referred to herein as SPARC MC, SPARC Mono, SPARC m or the like), and an anti-SPARC polyclonal antibody (referred to herein as SPARC PC, SPARC Poly, SPARC p or the like), Given the results of the previous analysis, the sample is further analyzed with relevant marker panels (4411). The sample is classified as HER2+ (4412), Triple Negative (4416), or ER/PR+, HER2− (4418). Further analysis depends on whether prior analysis determined that the sample should undergo “complete” biomarker panel analysis or a “tumor-specific” biomarker panel analysis. Tumor-specific analysis is performed for any cancer with a primary diagnosis, or first line, second line or third line therapy. Complete biomarker analysis is indicated for cancers that are fourth line, metastatic or beyond. Complete is also performed if the therapeutic history of the cancer is unknown (and thus becomes the default). In this manner, unnecessary testing can be avoided. HER2+(4412) samples are further analyzed by FISH for CMYC and TOP2A (4413), by IHC for p95 for tumor-specific analysis or for BCRP, ERCC1, MGMT, P95, RRM1, TOP2A and TOPO1 for complete analysis (4414), and by sequencing for mutation analysis of PIK3CA (4415). Triple negative (4416) samples are analyzed by IHC for p95 for tumor-specific analysis or for BCRP, ERCC1, MGMT, P95, RRM1, TOP2A and TOPO1 for complete analysis (4417). ER/PR+, HER2− (4418) samples are further analyzed by FISH for CMYC (4419), by IHC for p95 for tumor-specific analysis or for BCRP, ERCC1, MGMT, P95, RRM1, TOP2A and TOPO1 for complete analysis (4420). The results of the analysis are used to identify a therapeutic for the individual. The workflow can be generalized for the analysis of other diseases and tumor types.

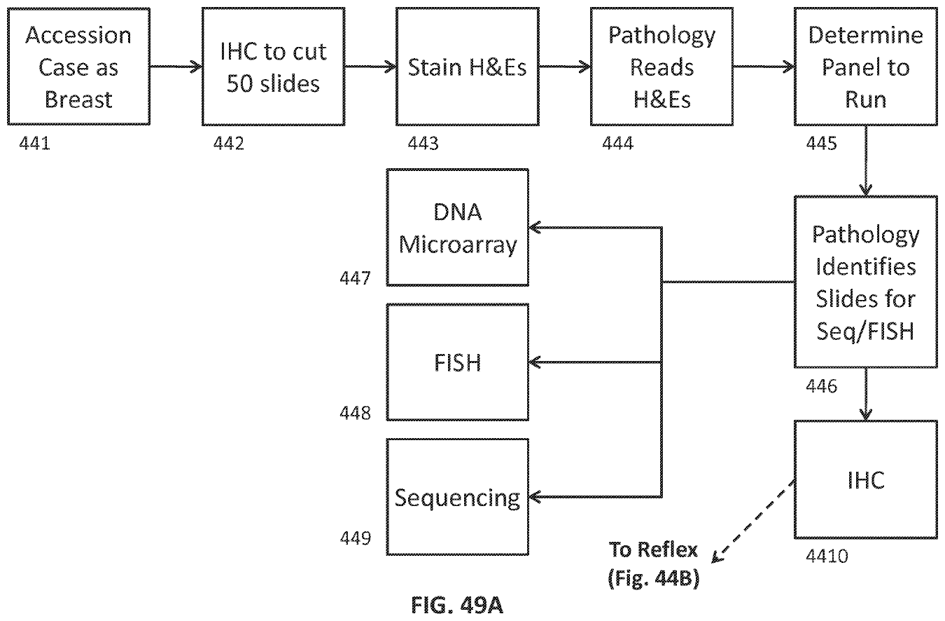

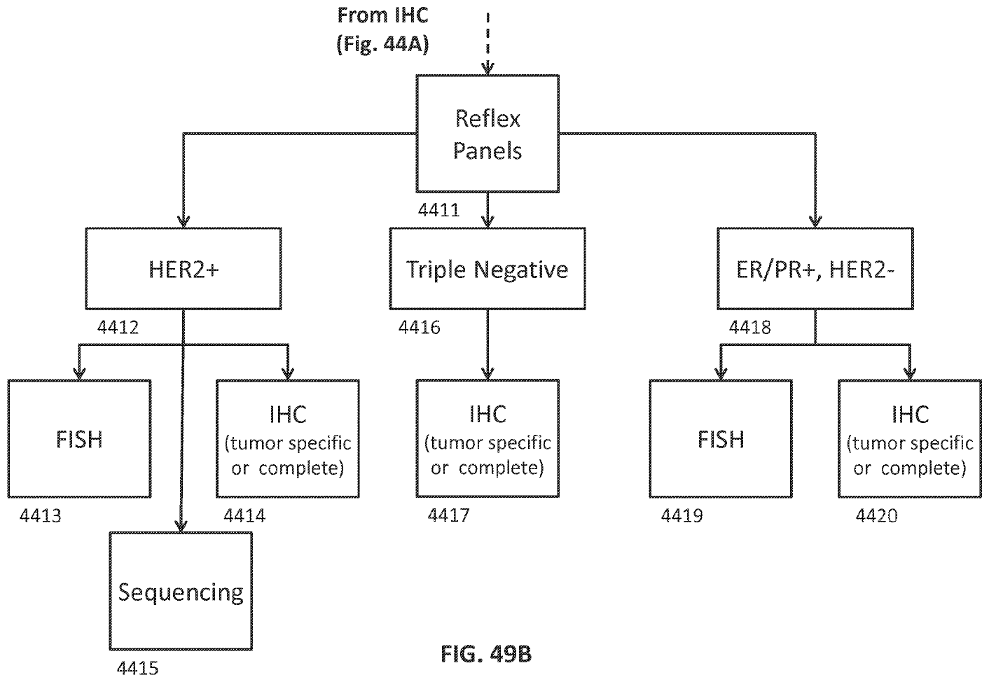

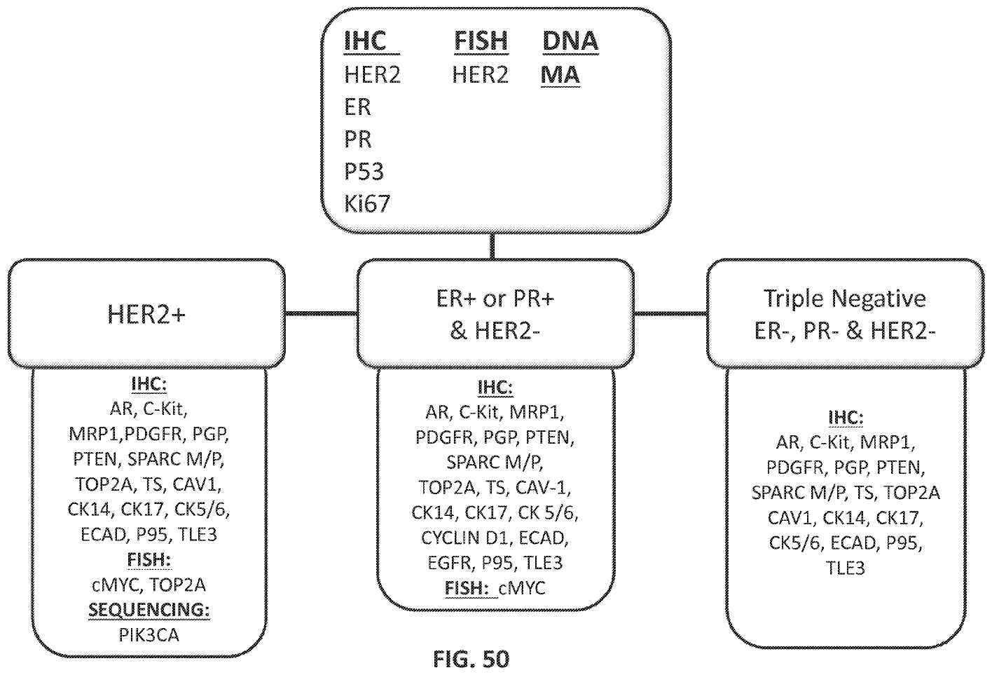

FIG. 50 and Table 35 illustrate a biomarker centric view of the workflow described above. In FIG. 34, initial IHC and FISH results on the indicated biomarkers is used to characterize the cancer as HER2+, Triple Negative, or ER/PR+, HER2−. The characterization guides the additional IHC, FISH and sequencing analysis that is performed. “DNA MA” indicates that a DNA microarray is performed on all samples that meet the quality threshold as described herein. DNA microarray analysis can be performed on a whole genome scale, with focus on genes that are informative for therapeutic treatment options, including at least ABCC1, ABCG2, ADA, AR, ASNS, BCL2, BIRC5, BRCA1, BRCA2, CD33, CD52, CDA, CES2, DCK, DHFR, DNMT1, DNMT3A, DNMT3B, ECGF1, EGFR, EPHA2, ERBB2, ERCC1, ERCC3, ESR1, FLT1, FOLR2, FYN, GART, GNRH1, GSTP1, HCK, HDAC1, HIF1A, HSP90AA1, IGFBP3, IGFBP4, IGFBP5, IL2RA, KDR, KIT, LCK, LYN, MET, MGMT, MLH1, MS4A1, MSH2, NFKB1, NFKB2, NFKBIA, OGFR, PARP1, PDGFC, PDGFRA, PDGFRB, PGP, PGR, POLA1, PTEN, PTGS2, PTPN12, RAF1, RARA, RRM1, RRM2, RRM2B, RXRB, RXRG, SIK2, SPARC, SRC, SSTR1, SSTR2, SSTR3, SSTR4, SSTR5, TK1, TNF, TOP1, TOP2A, TOP2B, TXNRD1, TYMS, VDR, VEGFA, VHL, YES1, and ZAP70. IHC is run on selected sections to analyze expression of biomarkers including AR, c-kit, CAV-1, CK 5/6, CK14, CK17, ECAD, ER, Her2/Neu, Ki67, MRP1, P53, PDGFR, PGP, PR, PTEN, SPARC, TLE3 and TS. Table 35 outlines shows the criteria used to perform additional assays. Tumor-specific analysis is used in the case of cancer with a primary diagnosis, or first line, second line or third line therapy. Complete biomarker analysis is indicated for cancers that are fourth line, metastatic or beyond.

TABLE 35
Additional Assays

| | Tumor-Specific | Complete |
|---|---|---|
| Criteria | Primary diagnosis | Fourth line therapy |
| | First line therapy | Metastatic |
| | Second line therapy | Therapeutic history unknown |
| | Third line therapy | |
| Additional Testing | | IHC: BCRP, ERCC1, |
| | | MGMT, RRM1, TOPO1 |
| | | FISH: EGFR |

Table 35 indicates prognostic markers in the breast cancer profiling. The markers used in the profiling can be used for theranostic (e.g., to guide selection of a candidate therapeutic) and prognostic purposes. “Y” in the “Prognostic?” column indicates that the marker can indicate a prognosis. Further details are described herein.

TABLE 35
Prognostic Breast Cancer Profiling

| | | | | Triple | ER/PR+/ |
|---|---|---|---|---|---|
| | | | HER2+ | Neg | HER2− |
| Biomarker | Method | Prognostic? | Profile | Profile | Profile |
| AR | IHC | | Y | Y | Y |
| Caveolin-1 | IHC | Y | Y | Y | Y |
| CK 14 | IHC | Y | Y | Y | Y |
| CK 17 | IHC | Y | Y | Y | Y |
| CK 5/6 | IHC | Y | Y | Y | Y |
| c-Kit | IHC | Y | Y | Y | Y |
| cMYC | FISH | Y | Y | | Y |
| Cyclin D1 | IHC | Y | | | Y |
| ECAD | IHC | Y | Y | Y | Y |
| EGFR | IHC | Y | | | Y |
| ER | IHC | | Y | Y | Y |
| (ESR1) | | | | | |
| HER2 | IHC/FISH | | Y | Y | Y |
| (ERBB2) | | | | | |
| Ki67 | IHC | | Y | Y | Y |
| MRP1 | IHC | | Y | Y | Y |
| (ABCC1) | | | | | |
| P53 | IHC | Y | Y | Y | Y |
| P95 | IHC | | Y | Y | Y |
| PDGFR | IHC | Y | Y | Y | Y |
| PGP | IHC | | Y | Y | Y |
| (ABCB1) | | | | | |
| PI3K | SEQ | | Y | | |
| PR | IHC | | Y | Y | Y |
| PTEN | IHC | | Y | Y | Y |
| SPARC | IHC | | Y | Y | Y |
| TLE3 | IHC | | Y | Y | Y |
| TOP2A | FISH | | Y | | |
| TOP2A | IHC | | Y | Y | Y |
| TS | IHC | | Y | Y | Y |
| (TYMS) | | | | | |

Table 37 provides illustrative candidate treatments corresponding to the molecular profiling described in this Example. In the table, a positive result for the indicated biomarker using the indicated techniaue guides selection of the corresponding therapeutic agent, or that of a related agent.

TABLE 37
Illustrative Drug-biomarker Associations

| Drug | Method | Biomarker(s) |
|---|---|---|
| 5-fluorouracil | DNA Microarray | TYMS |
| | IHC | TS |
| aminoglutethimide | DNA Microarray | ESR1, PR |
| | IHC | ER, PR |
| anastrozole | DNA Microarray | ESR1, PR |
| | IHC | ER, PR |
| capecitabine | DNA Microarray | TYMS |
| | IHC | TS |
| doxorubicin | DNA Microarray | ABCB1, TOP2A |
| | FISH | HER2, TOP2A |
| | IHC | PGP, TOP2A |
| epirubicin | DNA Microarray | ABCB1, TOP2A |
| | FISH | HER2, TOP2A |
| | IHC | PGP, TOP2A |
| exemestane | DNA Microarray | ESR1, PR |
| | IHC | ER, PR |
| fulvestrant | DNA Microarray | ESR1, PR |
| | IHC | ER, Ki67, PR |
| gonadorelin | DNA Microarray | PR |
| goserelin | DNA Microarray | PR |
| irinotecan | IHC | TOPO1 |
| lapatinib | FISH | HER2 |
| | IHC | HER2 |
| letrozole | DNA Microarray | ESR1, PR |
| | IHC | ER, PR |
| leuprolide | DNA Microarray | PR |
| liposomal- | DNA Microarray | ABCB1, TOP2A |
| doxorubicin | FISH | HER2, TOP2A |
| | IHC | PGP, TOP2A |
| medroxyprogesterone | DNA Microarray | ESR1, PR |
| | IHC | ER, PR |
| megestrol acetate | DNA Microarray | ESR1, PR |
| | IHC | ER, PR |
| methotrexate | DNA Microarray | ABCC1, DHFR |
| | IHC | MRP1 |
| nab-paclitaxel | DNA Microarray | SPARC |
| | IHC | SPARC mono, |
| | | SPARC poly |
| pemetrexed | DNA Microarray | DHFR, GART, TYMS |
| | IHC | TS |
| tamoxifen | DNA Microarray | ESR1, PR |
| | IHC | ER, Ki67, PR |
| taxanes | IHC | TLE3 |
| toremifene | DNA Microarray | ESR1, PR |
| | IHC | ER, Ki67, PR |
| trastuzumab | FISH | HER2 |
| | IHC | HER2, P95, PTEN |
| | Mutation (sequence | PIK3CA |
| | analysis) | |

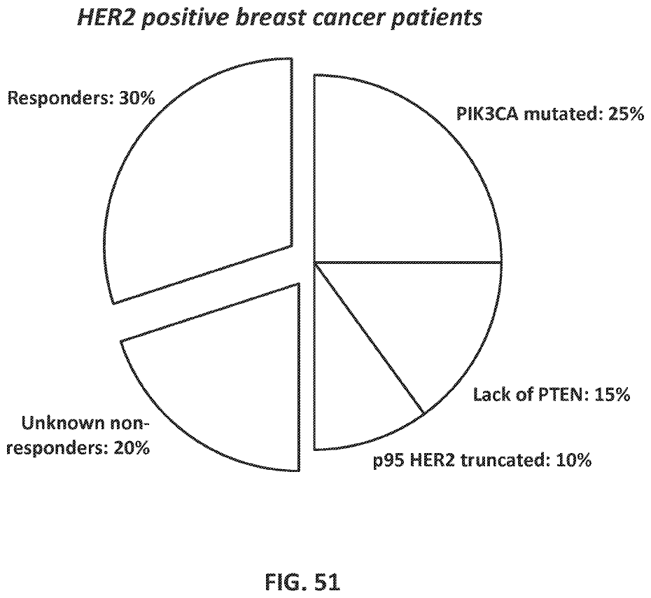

An illustrative benefit of the molecular profiling approach is illustrated in FIG. 51. For every 100 HER2+ patients, only about 30 (30%) will be Responders to treatment with trastuzumab. Molecular profiling according to the Example identifies 50 (50%) out of the 70 patients (70%) not likely to respond, e.g., because of PIK3CA mutations (25%), lack of PTEN (15%) or a p95 HER2 truncation (10%). HER2 spans the cell membrane and trastuzumab binds the external portion of the protein. However, most HER2 tests, including the FDA approved tests available from Dako (Dako North America, Inc., Carpinteria, Calif.) and Ventana (Ventana Medical Systems, Inc., Tucson, Ariz.), target the internal domain of HER2. Profiling according to the invention uses two antibodies for HER2: one with affinity to the internal domain, another with affinity to both the internal and external domains. If the latter antibody is negative but the tests targeting the internal domain are positive (e.g., the FDA approved tests), then HER2 is “p95 truncated” and trastuzumab will not be effective. By identifying patients unlikely to respond, efficacy of trastuzumab for a selected population can be increased from 30% to 60%. Furthermore, the molecular profiling methods of the invention can identify candidate treatments that are more likely to be effective in the trastuzumab non-responders.

Illustrative reports generated by the system are shown in International PCT Patent Application PCT/US2010/000407, filed Feb. 11, 2010; and International PCT Patent Application PCT/US2010/54366, filed Oct. 27, 2010, each of which application is incorporated herein by reference in its entirety.

### Example 4: Biomarker—Drug Associations and Lineage—Drug Associations

Table 38 lists exemplary associations between biomarkers and drugs associated with the biomarkers. When the biomarkers are found to be overexpressed in a patient sample, the drugs are indicated for use in treating the patient as described herein. For each drug, an indication is given of exemplary techniques that can be used to assess the corresponding biomarker. One of skill will appreciate that any technique can be used as described herein or known in the art, including without limitation microarray, PCR, IHC, ISH, FISH, and/or sequence analysis. Abbreviations in the table include the following: GE—Gene expression (e.g., RT-PCR; DNA microrarray); MA—Mutational analysis; IHC—Immunohistochemistry; FISH—Fluorescent in situ hybridization

TABLE 38
Biomarker - Drug Associations

| Biomarker | Drug Associations |
|---|---|
| ABCC1 (MRP1) | doxorubicin (IHC and GE), epirubicin (IHC and GE), |
| | methotrexate (IHC and GE), vincristine (IHC and GE), |
| | vinorelbine (IHC and GE), vinblastine (IHC and GE), |
| | etoposide (IHC and GE) |
| ABCG2 (BCRP) | cisplatin (IHC and GE)), carboplatin (IHC and GE) |
| ADA | pentostatin (GE), cytarabine (GE) |
| ALK (e.g., | crizotinib (FISH), pemetrexed (FISH) |
| EML4-ALK) | |
| AR | bicalutamide (IHC and GE), flutamide (IHC and GE), |
| | abarelix (GE), goserelin (GE), leuprolide (GE), |
| | gonadorelin (GE) |
| ASNS | asparaginase (GE), pegaspargase (GE) |
| BRCA1 | mitomycin (GE), cisplatin (GE), carboplatin (GE) |
| BRCA2 | mitomycin (GE), cisplatin (GE), carboplatin (GE) |
| CD52 | alemtuzumab (IHC and GE) |
| CDA | cytarabine (GE) |
| CES2 | irinotecan (GE) |
| DCK | gemcitabine (GE), cytarabine (GE) |
| DHFR | methotrexate (GE), pemetrexed (GE) |
| DNMT1 | azacitidine (GE), decitabine (GE) |
| DNMT3A | azacitidine (GE), decitabine (GE) |
| DNMT3B | azacitidine (GE), decitabine (GE) |
| EGFR | gefitinib (FISH and MA), erlotinib (FISH and MA), |
| | cetuximab (FISH and MA), panitumumab (FISH |
| | and MA) |
| EPHA2 | dasatinib (GE) |
| ERBB2 (HER2) | trastuzumab (IHC and FISH), lapatinib (IHC and |
| | FISH), doxorubicin (FISH), epirubicin (FISH), |
| | liposomal-doxorubicin (FISH) |
| ERCC1 | cisplatin (IHC and GE), carboplatin (IHC and GE), |
| | oxaliplatin (IHC and GE) |
| ER | tamoxifen (IHC and GE), toremifene (GE), |
| | fulvestrant (GE), anastrozole (IHC and GE), |
| | letrozole (IHC and GE), exemestane (GE), |
| | aminoglutethimide (GE), megestrol (GE), |
| | medroxyprogesterone (GE) |
| FLT1 (VEGFR1) | bevacizumab (GE), sunitinib (GE), sorafenib (GE) |
| GART | pemetrexed (GE) |
| HIF1A | sunitinib (GE), sorafenib (GE) |
| IGFBP3 | letrozole (GE) |
| IGFBP4 | letrozole (GE) |
| IGFBP5 | letrozole (GE) |
| KDR (VEGFR2) | sunitinib (GE), sorafenib (GE) |
| Ki67 | “tamoxifen + chemotherapy” (IHC) - breast only |
| KIT (cKIT) | sunitinib (MA and GE), sorafenib (GE), imatinib |
| | (MA and GE), dasatinib (MA and GE) |
| KRAS | gefitinib (MA), erlotinib (MA), cetuximab (MA), |
| | panitumumab (MA), sorafenib (MA), combination |
| | therapy (VBMCP) (MA) |
| cMET/MET | gefitinib (FISH), erlotinib (FISH) |
| MGMT | temozolomide (IHC and GE) |
| PDGFRA | sunitinib (GE), sorafenib (GE) |
| PDGFRB | sunitinib (GE), sorafenib (GE) |
| PGP (ABCB1) | doxorubicin (IHC and GE), liposomal doxorubicin |
| | (IHC and GE), epirubicin (IHC and GE), etoposide |
| | (IHC and GE), teniposide (GE), docetaxel (IHC and |
| | GE), paclitaxel (IHC and GE), vincristine (IHC and |
| | GE), vinorelbine (IHC and GE), vinblastine (IHC |
| | and GE) |
| PIK3CA/PI3K | cetuximab (MA), panitumumab (MA), trastuzumab |
| | (MA) |
| PR | tamoxifen (IHC and GE), toremifene (GE), fulvestrant |
| | (GE), anastrozole (IHC and GE), letrozole (IHC and |
| | GE), exemestane (GE), aminoglutethimide (GE), |
| | goserelin (GE), leuprolide (GE), gonadorelin (GE), |
| | megestrol (GE), medroxyprogesterone (GE) |
| PTEN | erlotinib (IHC), gefitinib (IHC), cetuximab (IHC), |
| | panitumumab (IHC), trastuzumab (IHC) |
| PTGS2 (COX2) | celecoxib (IHC and GE), aspirin (IHC) |
| BRAF1 (BRAF) | cetuximab (MA), panitumumab (MA) |
| RARA | ATRA (GE) |
| RRM1 | gemcitabine (IHC and GE), hydroxyurea (GE) |
| RRM2 | gemcitabine (GE), hydroxyurea (GE) |
| RRM2B | gemcitabine (GE), hydroxyurea (GE) |
| RXRB | bexarotene (GE) |
| SPARC | nab-paclitaxel (IHC and GE) |
| (mono/poly) | |
| SRC | dasatinib (GE) |
| SSTR2 | octreotide (GE) |
| SSTR5 | octreotide (GE) |
| TLE3 | paclitaxel (IHC), docetaxel (IHC) |
| TOPO1/TOP1 | irinotecan (IHC and GE), topotecan (IHC and GE) |
| TOPO2A/TOP2A | doxorubicin (IHC, FISH and GE), liposomal |
| | doxorubicin (IHC, FISH and GE), epirubicin |
| | (IHC, FISH and GE) |
| TOP2B | doxorubicin (GE), liposomal doxorubicin (GE), |
| | epirubicin (GE) |
| TUBB3 | paclitaxel (IHC), docetaxel (IHC), vinorelbine |
| | (IHC) |
| TS/TYMS | pemetrexed (IHC and GE), capecitabine (GE), |
| | fluorouracil (IHC and GE) |
| VDR | choleciferol (GE), calcitriol (GE) |
| VHL | sunitinib (GE), sorafenib (GE) |

### Example 5: HER2 Overexpression in Various Tumors

Testing for HER2 assists in the management of breast cancer and gastro-esophageal junction (GEJ) cancer. The purpose of this Example was to capture the relative frequency and distribution of HER2 overexpression in other cancers.

In a cohort of 11,223 patient samples, HER2 was assayed by immunohistochemistry using the Ventana HER2 (4B5) antibody. Slides were read by pathologists using the cutoff of (>=3+ and >=30% as positive) for HER2. In this same cohort, 2,246 patient samples underwent HER2 by FISH testing using Abbott's Pathvysion HER2 assay. FISH analysis was interpreted by a cytogeneticist based on ASCO/CAP guidelines for breast cancer. Prior to the analysis, 4,116 patient samples were excluded secondary to an unknown tumor lineage or other rarely observed lineages. In all, twenty-seven tumor lineages comprising 7,107 patient specimens were analyzed.

The frequency of HER2 by IHC was highest in breast carcinoma (68.6%), colorectal adenocarcinoma (7.2%), ovarian surface epithelial carcinoma (5.4%), gastroesophageal adenocarcinoma (4.5%), non-small cell lung cancer (3.1%), pancreatic adenocarcinoma (1.3%) and gastric adenocarcinoma (1.3%). In these same lineages, frequency of HER2 by FISH was highest in breast carcinoma (46.0%) followed by surface epithelial ovarian carcinoma (12.7%), non-small cell lung cancer (8.3%), gastroesophageal adenocarcinoma (6.7%), gastric adenocarcinoma (4.8%), pancreatic adenocarcinoma (4.0%), and colorectal adenocarcinoma (3.2%). Distributional differences in IHC versus FISH results were analyzed by Pearson's chi-square (×2) test, with statistical significance (p<0.05) achieved in breast carcinoma, gastric adenocarcinoma, gastroesophageal adenocarcinoma and surface epithelial ovarian carcinoma.

HER2 status was investigated in a large patient pool with advanced malignancies in a single clinical laboratory with standardized IHC and FISH. This study shows that HER2 is frequently expressed in multiple cancer types, which merits the inclusion of therapeutic strategies using HER2-targeted therapy in many types of tumors.

### Example 6: Molecular Profiling of Metastatic Breast Cancer in Body Cavity Fluids

The diagnosis of malignant effusion signifies disease progression and is associated with a worse prognosis regardless of tumor origin. Cancer cells in fluids have genotypic and phenotypic characteristics that differ from the primary tumor. This Example reports the molecular profiling for breast cancer metastasis in pleural and peritoneal fluids.

Malignant fluid samples submitted for molecular profiling were retrospectively identified. A cell-block was prepared or available for testing for all samples. An H&E slide was prepared from the cell-block and reviewed by a pathologist before further testing. Malignant cell percentages were determined for purpose of DNA microarray and sequencing. The results were reviewed and data compiled to calculate the yield of various molecular predictive tests.

28 metastatic breast cancer fluid samples were identified. Of the 28 cases, 10 biomarkers by IHC could be performed in 20 samples (71.4%), 1-9 in 1 sample (3.5%), while 7 samples were insufficient quality for IHC (25%). DNA microarray analysis was performed for 10 cases (35.7%). FISH was performed for EGFR in 7 cases (25%), Her2 Neu FISH was performed for 11 cases (39%), cMYC FISH was performed for 5 samples (17.8%) and TOPO2a by FISH in was performed for 3 samples (10.7%). Combined IHC/FISH and MA data was available in 10 cases, IHC and FISH data in 11 cases and IHC and MA data in 10 cases. Combined results of predictive markers provided information on therapeutic guidance according to the workflow presented in Example 3.

Molecular profiling of malignant fluids offers additional opportunities for testing those patients where other tissue samples such as needle core biopsy or resection samples are not available. Molecular profiling of fluids provides insight into the molecular characteristics of malignant cells in body cavity fluids and associated expression of unique therapeutic targets.

### Example 7: Molecular Profiling System

A system for carrying out molecular profiling according to the invention comprises the components used to perform molecular profiling on a patient sample, identify potentially beneficial and non-beneficial treatment options based on the molecular profiling, and return a report comprising the results of the analysis to the treating physician or other appropriate caregiver.

Formalin-fixed paraffin-embedded (FFPE) are reviewed by a pathologist for quality control before subsequent analysis. Nucleic acids (DNA and RNA) are extracted from FFPE tissues after microdissection of the fixed slides. Nucleic acids are extracted using phenol-chlorform extraction or a kit such as the QIAamp DNA FFPE Tissue kit according to the manufacturer's instructions (QIAGEN Inc., Valencia, Calif.).

Polymerase chain reaction (PCR) amplification is performed using the ABI Veriti Thermal Cycler (Applied Biosystems, cat #9902). PCR is performed using the Platinum Taq Polymerase High Fidelity Kit (Invitrogen, cat #11304-029). Amplified products can be purified prior to further analysis with Sanger sequencing, pyrosequencing or the like. Purification is performed using CleanSEQ reagent, (Beckman Coulter, cat #000121), AMPure XP reagent (Beckman Coulter, cat # A63881) or similar. Sequencing of amplified DNA is performed using Applied Biosystem's ABI Prism 3730xl DNA Analyzer and BigDye® Terminator V1.1 chemistry (Life Technologies Corporation, Carlsbad, Calif.). The BRAF V600E mutation is assessed using the FDA approved Cobas® 4800 BRAF V600 Mutation Test from Roche Molecular Diagnostics (Roche Diagnostics, Indianapolis, Ind.). NextGeneration sequencing is performed using the MiSeq platform from Illumina Corporation (San Diego, Calif., USA) according to the manufacturer's recommended protocols.

For RFLP, ALK fragment analysis is performed on reverse transcribed mRNA isolated from a formalin-fixed paraffin-embedded tumor sample using FAM-linked primers designed to flank and amplify EML4-ALK fusion products. The assay is designed to detect variants v1, v2, v3a, v3b, 4, 5a, 5b, 6, 7, 8a and 8b. Other rare translocations may be detected by this assay; however, detection is dependent on the specific rearrangement. This test does not detect ALK fusions to genes other than EML4.

IHC is performed according to standard protocols. IHC detection systems vary by marker and include Dako's Autostainer Plus (Dako North America, Inc., Carpinteria, Calif.), Ventana Medical Systems Benchmark® XT (Ventana Medical Systems, Tucson, Ariz.), and the LeicalVision Biosystems Bond System (Leica Microsystems Inc., Bannockburn, Ill.). All systems are operated according to the manufacturers' instructions.

FISH is performed on formalin-fixed paraffin-embedded (FFPE) tissue. FFPE tissue slides for FISH must be Hematoxylin and Eosion (H & E) stained and given to a pathologist for evaluation. Pathologists will mark areas of tumor to be FISHed for analysis. The pathologist report must show tumor is present and sufficient enough to perform a complete analysis. FISH is performed using the Abbott Molecular VP2000 according to the manufacturer's instructions (Abbott Laboratories, Des Plaines, Iowa). ALK is assessed using the Vysis ALK Break Apart FISH Probe Kit from Abbott Molecular, Inc. (Des Plaines, Ill.). HER2 is assessed using the INFORM HER2 Dual ISH DNA Probe Cocktail kit from Ventana Medical Systems, Inc. (Tucson, Ariz.) and/or SPoT-Light® HER2 CISH Kit available from Life Technologies (Carlsbad, Calif.).

### Example 8: Molecular Profiling Reports

Exemplary reports generated by the molecular profiling systems and methods of the invention are shown in FIGS. 37-39.

FIGS. 37A-37Y illustrate an exemplary patient report based on molecular profiling for an ovarian cancer. The molecular profile used was a molecular intelligence (MI) profile for a high grade glioma (see FIGS. 33O-P and Tables 19, 21 and accompanying text) and included mutational analysis on a panel of 34 genes performed by Next Generation sequencing. FIG. 37A illustrates a cover page of a report indicating patient and specimen information for the glioma patient. FIG. 37A also displays a summary of agents associated with potential benefit or potential lack of benefit. Agents associated with potential benefit are further annotated as on NCCN Compendium™ (i.e., recommended by NCCN guidelines for the particular tumor lineage) or off NCCN Compendium™ (i.e., not part of the NCCN guidelines for the particular tumor lineage). FIG. 37A also lists clinical trials which may be available given the molecular profiling results, here no trials were matched. FIG. 37B reports further patient and specimen information for the glioma patient. FIG. 37C illustrates more detailed information for biomarker profiling used to associate agents with potential benefit. FIG. 37D illustrates more detailed information for biomarker profiling used to associate agents with lack of potential benefit. FIG. 37E illustrates more detailed information for biomarker profiling used to associate agents with indeterminate benefit. FIG. 37F and FIG. 37G illustrate more detailed information for mutational analysis performed by Next Generation Sequencing. This section indicates mutations that were identified (FIG. 37F) as well as providing a list of genes that were tested without alterations (FIG. 37G). FIG. 37H, FIG. 37I, FIG. 37J and FIG. 37K provide a listing of published references used to provide evidence of the biomarker—agent association rules used to construct the report. FIG. 37L presents a disclaimer, e.g., that ultimate treatment decisions reside solely within the discretion of the treating physician. FIG. 37M provides a cover page for an Appendix to the report. FIG. 37N and FIG. 37O provide more information about the mutational analysis performed by Next Generation sequencing, Sanger sequencing, pyrosequencing, EGFR RFLP analysis, Cobas BRAF V600E analysis and MGMT Methyltion analysis. Depending on the tumor lineage, not all of these tests are necessarily performed. FIG. 37P provides more information about the IHC analysis performed on the patient sample, e.g., the staining results and threshold for each marker. FIG. 37Q provides more information about the ISH analysis performed on the patient sample, which comprised CISH for this tumor. FIG. 37R, FIG. 37S, FIG. 37T, FIG. 37U, FIG. 37V, FIG. 37 W, and FIG. 37X provide a description of the biomarkers assessed per the molecular profiling. FIG. 37Y provides the framework used for the literature level of evidence as included in the report.

FIGS. 38A-38AA illustrate an exemplary patient report based on molecular profiling for a lung adenocarcinoma. The molecular profile used was a molecular intelligence (MI) profile for lung cancer (see FIGS. 33I-J, Table 17 and Table 18) and included mutational analysis on a panel of 34 genes performed by Next Generation sequencing (“NGS Panel,” see Table 25 and accompanying text). The sections of the report are the same as those described for FIGS. 37A-Y as adapted for lung cancer molecular profiling. FIG. 38A illustrates a cover page of a report indicating patient and specimen information for the lung cancer patient. FIG. 38A also displays a summary of agents associated with potential benefit or potential lack of benefit. Agents associated with potential benefit are further annotated as on NCCN Compendium™ (i.e., recommended by NCCN guidelines for the particular tumor lineage) or off NCCN Compendium™ (i.e., not part of the NCCN guidelines for the particular tumor lineage). FIG. 38A also lists clinical trials which may be available given the molecular profiling results, here trials were matched based on cMET status. FIG. 38B reports further patient and specimen information for the lung cancer patient. FIG. 38C and FIG. 38D illustrate more detailed information for biomarker profiling used to associate agents with potential benefit. FIG. 38E illustrates more detailed information for biomarker profiling used to associate agents with lack of potential benefit. FIG. 38F illustrates more detailed information for biomarker profiling used to associate agents with indeterminate benefit. FIG. 38G and FIG. 38H illustrate more detailed information for mutational analysis performed by Next Generation Sequencing. This section indicates mutations that were identified (FIGS. 38G-H) as well as providing a list of genes that were tested without alterations (FIG. 38H). FIG. 38I provides a listing of clinical trials matched to cMET. FIG. 38J, FIG. 38K, FIG. 38L and FIG. 38M provide a listing of published references used to provide evidence of the biomarker—agent association rules used to construct the report. FIG. 38N presents a disclaimer, e.g., that ultimate treatment decisions reside solely within the discretion of the treating physician. FIG. 38O provides a cover page for an Appendix to the report. FIG. 38P provides more information about the mutational analysis performed by Next Generation sequencing. FIG. 38Q provides more information about the IHC analysis performed on the patient sample, e.g., the staining results and threshold for each marker. FIG. 38R provides more information about the FISH analysis performed on the patient sample. FIG. 38S provides more information about the CISH analysis performed on the patient sample. FIG. 38T, FIG. 38U, FIG. 38V, FIG. 38 W, FIG. 38X, FIG. 38Y, and FIG. 38Z provide a description of the biomarkers assessed per the molecular profiling. FIG. 38AA provides the framework used for the literature level of evidence as included in the report.

FIGS. 39A-39Y illustrate an exemplary patient report based on molecular profiling for a non-small cell lung cancer with stand alone mutational analysis performed by Next Generation sequencing (“NGS Panel,” see Table 25 and accompanying text). FIG. 39A presents an overview of the patient history and sample. FIG. 39B displays a summary of agents associated with potential benefit or potential lack of benefit. Agents associated with potential benefit are further annotated as on NCCN Compendium™ (i.e., recommended by NCCN guidelines for the particular tumor lineage) or off NCCN Compendium™ (i.e., not part of the NCCN guidelines for the particular tumor lineage). FIG. 39B also lists clinical trials which may be available given the molecular profiling results, here trials were matched based on status of PIK3CA, ALK, BRAF, KRAS, EGFR, and GNA11. FIG. 39C illustrates more detailed information for biomarker profiling used to associate agents with potential benefit. FIG. 39D illustrates more detailed information for biomarker profiling used to associate agents with lack of potential benefit. FIG. 39E, FIG. 39F and FIG. 39G illustrate more detailed information for mutations detected by Next Generation Sequencing. FIG. 39H provides a list of genes that were tested without finding alterations. FIG. 38I, FIG. 39J, FIG. 39K, FIG. 39L and FIG. 39M provide a listing of clinical trials matched to the gene alterations that were found. FIG. 39N and FIG. 39O provide a listing of published references used to provide evidence of the biomarker—agent association rules used to construct the report. FIG. 39P presents a disclaimer, e.g., that ultimate treatment decisions reside solely within the discretion of the treating physician. FIG. 38Q provides more information about the mutational analysis performed by Next Generation sequencing. FIG. 39R provides more information about the ISH analysis performed on the patient sample to assess gene rearrangements, which comprised FISH for this tumor. FIG. 39S, FIG. 39T, FIG. 39U, FIG. 39V, FIG. 39 W, and FIG. 39X provide a description of the biomarkers assessed per the molecular profiling. FIG. 39Y provides the framework used for the literature level of evidence as included in the report.

### Example 9: HER2 Status in Lung Non-Small Cell Carcinomas (NSCLC)

Between 10 and 30% of non-small cell lung cancers (NSCLC) harbor somatic activating mutations in the gene encoding the EGF receptor (EGFR). Tumors with the most common alterations, exon 19 deletions and exon 21 point mutations (L858R), are initially responsive to EGFR tyrosine kinase inhibitors (TKI) such as gefitinib or erlotinib, but eventually acquire resistance. Upon disease progression, more than half of the cases harbor a second-site mutation T790M in EGFR, which alters binding of drug to the ATP-binding pocket. Recently, HER2 amplification was recognized as a novel mechanism of acquired resistance that occurs in a subset of NSCLC lacking the EGFR T790M mutation. This Example investigated simultaneous occurrence of EGFR mutations and HER2 gene amplifications.

Molecular profiles of 2271 cases of non-small cell lung cancers obtained according to the methods herein were reviewed for Her2 protein expression via immunohistochemistry, HER2 gene amplification via FISH, and EGFR and KRAS gene mutations via Sanger sequencing.

The molecular profiling revealed that EGFR was mutated in 12%, and KRAS in 32% of cases. HER2 gene amplification (HER2/CEP17≥2.2) was detected in 4% of tested cases ( 22/589) associated with 3+ protein expression. Coexistence of HER2 gene amplification and EGFR mutation was identified in 3 cases (L858R, A859T and E746 A750del); while KRAS was mutated in 7 HER2-amplified cases.

HER2 gene amplification is a rare event in non-small cell carcinomas (4%). In no case was HER2 amplification associated with T790M mutation. Double EGFR mutations (L858R/T790M and E746_A750del/T790M) were however found in only 2 cases. NSCLC with HER2 amplification were frequently (39%) associated with KRAS activating mutation. A rare A859T mutation was found in one case and was associated with HER2 gene amplification. This mutation was previously associated with TKI resistance (Han S et al. JCO 2005; no knowledge of HER2 status), but this may be the effect of HER2 gene amplification and not the intrinsic property of the EGFR mutated protein. Since earlier studies have suggested that HER2 amplification may cause resistance to erlotinib and gefitnib, NSCLC patients with HER2 amplification and activating EGFR mutation may respond better to afatinib which inhibits HER2 in addition to EGFR.

In this Example, molecular profiling identified a group of patients that can be expected to have no benefit from targeted therapy aimed at mutated EGFR (tumors with exon 21 point mutations and exon 19 deletions) because they have HER2 gene amplification (and concomitant protein over expression). Molecular profiling also identified association of HER2 amplification with a rare EGFR mutation that was previously considered to be resistance causing, but in the light of these findings and recent literature, this is most likely the result of HER2 amplification.

When devising a targeted treatment strategy in NSCLC, biomarkers evaluation should be comprehensive in order to maximize the benefit and minimize potential side effects of drugs without expected benefits.

### Example 10: Integrating Molecular Profiling into Cancer Treatment Decision Making; Experience with Over 30,000 Cases

A limited number of well-defined genetic alterations determine cellular response to chemotherapy and these are the same across many cancer lineages. While these were all found in the common cancers, a limited amount of information exists that associates these geneti c alterations with rare cancers. Contrary to what their name implies, the “rare” cancers as a group constitute some 1/4 of all cancer burden. Also, in common cancer types we find new genetic alterations that may have theranostic potential

A number of genetic alterations determine cellular response to chemotherapy. These changes are termed actionable as evidenced by clinical studies showing associations with improved survival or objective response in tumors carrying specific molecular characteristics. Molecular profiling of both common and rare cancer types provides for the identification of potentially actionable targets for chemotherapy with many unexpected associations.

Results of molecular profiling as described herein were stored in a database of ≥30,000 patients. At least the following biomarkers were assessed for members of the cohort; immunohistochemical results of 12 protein biomarkers (PTEN, AR, ER, PR, ERCC1, PGP, RRM1, SPARCm, SPARCp, TOP2A, TOPO1, TS), fluorescent in-situ hybridization of 8 genes (HER2, c-MET, c-MYC, EGFR, PIK3CA, TOP2A, ALK, ROS1) and sequencing for somatic mutations in 8 genes (BRAE, KRAS, EGFR, PIK3CA, c-KIT, NRAS, GNA11, GNAQ).

All data was deidentified and reviewed for well-established driver gene mutations, gene copy number alterations, and protein expression patterns that are potentially relevant for selection of targeted therapy. Selected genetic abnormalities in each patient's tumor were associated with potential benefit or lack of benefit with specific therapeutic agents based on evidence that exists for such an association in the peer-reviewed medical literature. All relevant published studies were evaluated using the U.S. Preventive Services Task Force (“USPSTF”) grading scheme for study design and validity. Assay methodologies to evaluate tumor genetic abnormalities and their potential theranostic associations included sequencing (Sanger, pyrosequencing), PCR, FISH, CISH, and immunohistochemistry.

All common malignancies (10 most common solid tumor types in men and women) and 10 rare cancer types were represented in the study cohort (minimum of 100 cases and maximum of >4,000 cases in each individual cancer type). Well established driver mutations and protein expression patterns were identified in common cancers with expected frequencies (e.g. HER2 amplification in breast, PIK3CA mutations in ER+ breast cancer, EGFR mutations in NSCLC, etc.). Importantly, unexpected new potentially actionable targets were identified in common cancers (e.g. 6.7% HER2 amplification in NSCLC, 1.6% KRAS mutation in prostatic adenocarcinoma) and rare cancers (e.g. 8.3% ALK alteration in soft tissue sarcomas, 10.5% c-MET and 26.4% EGFR gene amplification in melanomas, 16.3% KRAS mutation cholangiocarcinomas, 10% AR expression in soft tissue sarcomas), as well in cancers of unknown primary site (CUPS; approximately 4% of all tested cases). Thus, molecular profiling according to the invention identified biomarker statuses that can be linked to actionable therapeutic agents in the expected cancer lineages and also in non-standard lineages.

This review of a large referral cancer molecular profiling database provided unparalleled insight into the distribution of common and rare molecular alterations with potential treatment implications. Numerous targets were discovered that had a potential to be treated by the conventional chemotherapy as well as targeted therapy not usually considered for the cancer lineage. Comparison between an individual patient tumor profile and database for the matched cancer type provides additional level of support for targeted treatment choices.

### Example 11: Biomarker Analysis of Glioblastoma and the Implications for Therapy

Glioblastoma multiform (GBM), or WHO grade IV malignant astrocytoma, represents the most prevalent and aggressive cancer from the central nervous system. GBM tumors are infiltrative in nature and often remain undetected until complete resection is impossible; therefore untreated patients usually die within 3 months of diagnosis. The challenges of GBM treatment are also presented by the involvement of multiple complex pathways underlying GBM cancer cell biology, which allows treated cancer cells to fast evolve and develop resistance; the drug delivery difficulty presented by the blood-brain barrier; and the regional heterogeneity of the tumor. Standard of care of GBM involves maximal safe surgical resection followed by a combination of radiation and chemotherapy with an oral DNA alkylating agent temozolomide, improving patient survival to approximately 14.6 months.

Almost all GBM patients experience recurrence, for which the standard treatment option is lacking, therefore novel treatment options are in great need. Research shows that distinct genetic events underlie the tumorigenesis and progression of symptomatically similar GBM subtypes, and thus full evaluation of patient's biomarker characteristics should guide treatment choices. A comprehensive genome-wide analysis for biomarkers in 206 GBMs by the Cancer Genome Atlas project identified key critical signaling pathways in GBM including RTK/Ras/PI3K s, p53 and RB, indicating that the genetic aberrations in these pathways may provide insight to guide molecularly targeted therapy. Recent GBM subtyping based on gene expression has shown importance in prognosis and predictivity of treatment response.

The DNA repair enzyme, O-6-methylguanine-DNA methyltransferase or MGMT, is one of the most important biomarkers in glioblastoma, the detection of which, through immunohistochemistry or promoter methylation analysis by pyrosequencing, is important in identifying GBM patients who can benefit from temozolomide. Patients with MGMT promoter methylation when treated with temozolomide have been reported to have median survivals of over 20 months. Clinical data show that 40% of GBM patients are >65 yrs old, presenting worse prognosis and that they are more frequently MGMT methylated. Evaluating MGMT status is particularly important in this patient cohort so that the non-responders identified can be spared the side effect of temozolomide. Presence of MGMT methylation also marks a mismatch repair (MMR)—deficient phenotype, and upon temozolomide treatment further selective pressure is introduced to lose mismatch repair function, causing a MMR-defective hypermutator phenotype, for which selective treatment strategy is needed to prevent the emergence of drug resistance. Thus, stratifying patients based on MGMT methylation status and profiling the biomarkers of each subgroup can suggest more effective combination therapy strategy.

Tumor profiling services using a multi-platform approach as described herein were used to provide a comprehensive analysis for glioblastoma patients and search for biomarker abnormalities in all key pathways identified. Biomarkers previously identified as important for GBM biology (MGMT, PTEN, BRAF) were interrogated through various techniques as described herein, along with over 30 additional biomarker abnormalities not typically suspected for GBM. See, e.g., FIGS. 33A-33B, Table 21 and Table 22. Biomarker analysis provides biomarker evidence to support drug usage that are common in treatment practice, but also proposes novel combination therapy strategies to tackle this challenging tumor type of glioblastoma. Through a thorough retrospective analysis on biomarker data, patient stratification are possible and trends of different treatment options tailored to patient's unique biomarker characteristics will be identified, thus shedding light on individualized medicine to treat this challenging disease.

Methodology and Results:

Biomarker data were analyzed from a cohort of 570 glioblastoma patients who received Tumor Profiling Services from 2009 to 2013 using the methods described herein. Test methodologies included IHC, FISH, CISH, Sanger sequencing, MGMT promoter methylation and NextGen Sequencing (Illumina TruSeq panel). Statistical tools including T-Test were used in analysis.

This study evaluated predictive biomarkers associated with NCCN-recommended therapeutic agents that provided clinicians with decision support in their selection of optimal chemotherapy. In our analysis, from the complete cohort of 570 patients, 492 had MGMT IHC testing, out of whom 344 (70%) patients had negative MGMT expression; 59% of a subgroup of 29 (17) patients were found to carry MGMT promoter methylation tested by pyrosequencing. This study thereby identified a patient subset that may respond better to alkylating agents including temozolomide. Negative ERCC1 expression was seen in 53% (201/376) patients and positive TOPO1 IHC was seen in 49% (178/367), indicating potential benefit from platinum agents cisplatin/carboplatin and irinotecan, respectively.

Biomarkers associated with other chemotherapies commonly used are also evaluated. TS was found to be under-expressed in 37% (132/360) patients, suggesting clinical benefit from fluorouracil. Drug pumps, PGP and MRP1, were overexpressed in 34/338 (10%) and 236/351 (67%) of patients, respectively, indicating possible resistance for their substrates, including etoposide, vinca alkaloids, and methotrexate.

Pathway assessment was performed by various molecular tests to stratify patients for targeted therapies. Ckit was overexpressed in 6.5% (5/77) patients and mutated in 5.6% (2/36), PDGFRA was overexpressed in 27% ( 57/211) of patients, indicating potential benefit from imatinib. Further, BRAF, KRAS, PIK3CA mutations and PTEN loss were found in 7.8% ( 11/142), 2.7% (4/149), 6.7% (8/120), and 9.6% ( 50/519) of patients, respectively, indicating activation of the RAS/RAF pathway and the PIK3CA/mTOR pathway.

Subgroup analysis showed that in patients with MGMT methylation (see Table 39), only 1 out of 19 patients (9%) overexpressed thymidylate synthase (TS), while in patients lacking MGMT methylation, 5 out of 8 patients (63%) overexpressed TS. The differential expression reached statistical significance (p=0.025) and indicated that fluoropyrimidines may be of potential benefit for patients presenting MGMT methylation. A similar trend was observed for RRM1 expression (36% vs. 75% for methylated vs. unmethylated), showing potential benefit of using gemcitabine for MGMT methylated patients.

TABLE 39
Subgroup analysis based on patient MGMT methylation status

| | MGMT methylated patients | | | | MGMT un-methylated patients | | | | p value |
|---|---|---|---|---|---|---|---|---|---|
| Biomarker | N | + | − | Positive Percentage | N | + | − | Positive Percentage | (t-test) |
| AR | 15 | 2 | 13 | 0.13 | 12 | 0 | 12 | 0.00 | 0.16 |
| cMET | 15 | 1 | 14 | 0.07 | 13 | 1 | 12 | 0.08 | |
| ER | 15 | 0 | 15 | 0.00 | 11 | 0 | 11 | 0.00 | |
| ERCC1 | 11 | 3 | 8 | 0.27 | 8 | 2 | 6 | 0.25 | |
| Her2 | 15 | 0 | 15 | 0.00 | 11 | 0 | 11 | 0.00 | |
| MGMT | 1 | 0 | 1 | 0.00 | 1 | 0 | 1 | 0.00 | |
| PR | 15 | 0 | 15 | 0.00 | 11 | 0 | 11 | 0.00 | |
| PTEN | 15 | 13 | 2 | 0.87 | 12 | 12 | 0 | 1.00 | 0.16 |
| RRM1 | 11 | 4 | 7 | 0.36 | 8 | 6 | 2 | 0.75 | 0.073 |
| SPARC mono | 15 | 1 | 14 | 0.07 | 12 | 4 | 8 | 0.33 | |
| SPARC poly | 15 | 4 | 11 | 0.27 | 12 | 2 | 10 | 0.17 | |
| Spare both | 15 | 5 | 10 | 0.33 | 12 | 5 | 7 | 0.42 | |
| TLE3 | 15 | 6 | 9 | 0.40 | 12 | 5 | 7 | 0.42 | |
| TOPO1 | 15 | 6 | 9 | 0.40 | 8 | 5 | 3 | 0.63 | 0.3341 |
| TS | 11 | 1 | 10 | 0.09 | 8 | 5 | 3 | 0.63 | 0.025 |
| ALK FA | 5 | 0 | 5 | 0.00 | 5 | 0 | 5 | 0.00 | |
| BRAF | 12 | 2 | 10 | 0.17 | 15 | 0 | 15 | 0.00 | 0.17 |
| cKIT SEQ | 11 | 0 | 11 | 0.00 | 11 | 0 | 11 | 0.00 | |
| KRAS | 12 | 1 | 11 | 0.08 | 14 | 0 | 14 | 0.00 | 0.34 |
| NRAS | 10 | 0 | 10 | 0.00 | 11 | 1 | 10 | 0.09 | |
| PIK3CA | 11 | 0 | 11 | 0.00 | 10 | 0 | 10 | 0.00 | |

Conclusions:

A retrospective biomarker analysis of 570 glioblastoma patients who received tumor profiling services from 2009 to date was performed to search for clinical implications to support the usages of treatment regimens within and outside of standard of care.

From the robust biomarker evaluation performed using multiple platforms, these data show that 59% of patients are good responders to temozolomide, 53% to platinum agents and 49% to irinotecan. These agents all have shown to cross blood brain barrier and are recommended by NCCN; evaluating biomarker status will substantially assist the clinician in treatment selection.

Various targeted therapies are in different phases of clinical trials and our data provide biomarker information to stratify patients into trials where they can benefit. About 7% of patients show PIK3CA mutation and 10% shows a loss of PTEN, indicating a constitutive activated PIK3CA pathway, therefore may benefit from mTor inhibitors. About 8% of patients are shown to have a BRAF mutation and 3% to harbor KRAS mutation, indicating the dysregulation of RAS/RAF/MEK/ERK pathway, presenting a potential benefit of targeted agents including MEK inhibitors. Multi-targeted tyrosine kinase inhibitors including imatinib are being extensively studied in clinical trials, and these data indicate that 6.5% patients overexpress cKIT, 27% overexpress PDGFRA and 5.6% carry a cKIT mutation, and can potentially benefit.

The substrates of drug pumps PGP and MRP1 are different but overlap. PGP and MRP1 were overexpressed in 10% and 67% of patients in this study, showing that evaluation of drug pump level may be important when common drugs etoposide, vincristine (substrate of both) or methotrexate (substrate of MRP1) is applied.

From a subgroup analysis based on patient MGMT methylation status, differential biomarker expression pattern is noticeable and the most significant result is from a distinct expression of thymidylate synthase between the two groups (1 in 11, or 9% in MGMT methylated cohort vs. 5 in 8, or 63% in MGMT unmethylated cohort, p=0.025). See Table 39. This result provides biomarker evidence to propose a novel combination therapy to treat MGMT methylated GBM patients by alkylating agents and fluoropyrimidines. Synergistic treatment effect of temozolomide and 5-FU pro-drug capecitabine has been reported in pancreatic endocrine carcinomas. Fluorouracil and capecitabine can readily cross blood brain barrier, and the data here support their usage in conjunction with temozolomide in MGMT methylated GBM patient cohort. This finding shows great potential of using biomarker data and evidence-based association to provide guidance for clinical research and eventually for clinical practice.

### Example 12: Use of Different Methodologies for Detecting EGFR Mutations in Selecting Chemotherapy for Patients with Lung Cancer

Mutation analysis of the kinase domain of EGFR (exons 18-21) is a standard recommended procedure for patients diagnosed with non-small cell lung cancer (NSCLC). NSCLC patients whose tumor harbors certain EGFR mutations have notable responses to EGFR inhibitors. Several different methodologies are available as published assays or commercially available kits and include Sanger Sequencing, allele specific PCR (ASP) and restriction fragment length polymorphism (RFLP). Differences in the design of these assays dictate which clinically relevant mutations will be detected.

Methods:

Sanger Sequencing of EGFR found 518 potentially clinically actionable mutations and 45 variants of unknown significance of the 4307 samples tested. To assess which clinically relevant EGFR mutations will be detected by the ASP and RFLP, we performed an in silico analysis of all observed mutations against the design specification of the ASP and RFLP assays. The RFLP assay used in this assessment was developed in our laboratory and is designed to detect all G719 mutations, all exon 19 deletions, all exon 20 insertions and the specific mutations T790M, L858R, L861R and L861Q. A commercially available ASP kit designed to detect 29 mutations in the kinase domain was also analyzed in this study.

Results:

Based on the performance characteristics assumed by the RFLP and ASP assays, the analytical sensitivities for detecting clinically actionable mutations of the two methodologies are 98.8% and 86.7% respectively, when compared to Sanger Sequencing. Among the 1.2% of mutations not detected by RFLP, the assay missed the S768I mutation (0% detected) and did not detect some G719 mutations, exon 19 deletions, and exon 20 insertions (76.3, 86.6 and 35.3% were detected, respectively). The ASP method did not detect 13.3% of the mutations detected by sequencing.

Conclusion:

Sequencing is still the preferred method of mutation detection in EGFR for NSCLC as it is the most comprehensive. However, if tumor nuclei are limited, then a more sensitive method than sequencing is required and, EGFR mutation detection by RFLP identifies more potentially clinically relevant mutations than ASP as the ASP method would produce false negative results in 13.5% of patients expected to respond to EGFR inhibitors.

### Example 13: Molecular Profiling Panels

FIGS. 34A-34C illustrate biomarkers assessed using a molecular profiling approach as outlined in FIGS. 33A-Q, Tables 7-25, and accompanying text herein. FIG. 34A illustrates biomarkers that are assessed. The row labeled MI Profile™ does not include the Next Generation sequencing panel. The row labeled MI Profile™ Plus includes the Next Generation sequencing panel. The biomarkers that are assessed according to the Next Generation sequencing panel are shown in FIG. 34B. FIG. 34C illustrates sample requirements that can be used to perform molecular profiling on a patient tumor sample according to the panels in FIGS. 34A-34B.

### Example 14: Assessment of cMET by IHC, FISH, and Next Generation Sequencing

cMET overexpression and/or activation have been implicated in signaling pathways that promote cell proliferation, invasion, and survival. cMET is an oncogenic driver in various malignancies and is a potential therapeutic target. This Example assesses the distribution of cMET expression by immunohistochemistry (IHC), cMET amplification by FISH, and cMET mutation by next generation sequencing (NGS) across a variety of tumor types. This Example further assesses the correlation of cMET across technology platforms as performed in a CLIA-certified oncology reference laboratory.

In a cohort of 9161 patient samples, cMET protein expression was assayed by IHC (NCL-cMET and SP44, 9161 samples), FISH (BAC clone, 7435 samples) and NGS (Illumina Truseq Amplicon—Cancer Panel, 3163 samples).

This analysis found the highest cMET expression rates in the following tumor types: pancreatic cancer (56%, 231 out of 411), cholangiocarcinoma (51%, 63 out of 123), extrahepatic bile duct cancer (50%), small intestinal cancer (49%), colorectal cancer (46%), uveal melanoma (43%), gastroesophageal cancer (36%), gastric cancer (34%), and non-small cell lung cancer (33%), and head and neck cancer (32%). The lowest expression rates of cMET by IHC included non-epithelial ovarian cancer (6%, 5/84), glioblastoma (6%, 11/198), neuroendocrine tumors (6%, 18/296), prostate cancer (7%) and soft tissue malignancies (9%). Analysis of cMET by FISH identified the highest levels amplification in peritoneal/retroperitoneal sarcomas (9%, 2/23), non-small cell lung cancers (7%, 65/983), and melanoma (7%, 12/165). In 3163 samples tested by NGS platform, only 9 mutations were identified—all were variants of unknown significance and five were detected in non-small cell lung cancer specimens. The corresponding exon and protein changes in these five samples were as follows: D1028H (exon 14) in three samples; 6391A (exon 2) and 5203T (exon 2) in one sample each. The other four had the following mutations: 5203T (exon 2) and G391E (exon 2) in melanoma; 5203T (exon 2) in colorectal cancer; and K1121N (exon 16) in female genital tract malignancy. The highest percentage agreement between IHC and FISH was observed in the following lineages: pancreatic cancer (50.8%), cholangiocarcinoma (52.8%), colorectal cancer (64.5%), non-small cell lung cancer (66.1%), and gastric cancer (67.0%). Of those specimens with mutated cMET, five of nine were positive by IHC and none by FISH.

The data in the Example show that cMET overexpression and/or activation is prevalent in various malignancies. Ongoing clinical trials targeting cMET suggest that efforts should be made to accurately interrogate tumors for cMET testing. As shown by the FISH-IHC concordance data, cMET analysis is enhanced when assessed using multiple technologies.

### Example 15: Molecular Profiling in Gastric Cancer

Current NCCN guidelines recommend perioperative epirubicin (E), cisplatin (C), and 5-fluorouracil (F) along with other triple agent derivations as first line therapeutic approaches for operable gastric adenocarcinoma (GC). In this Example, molecular profiling was used to evaluate chemotherapy targeted biomarkers associated with ECF therapy for GC.

Surgically obtained GC specimens were analyzed by immunohistochemistry for TOP2A, TS, and ERCC1 expression as described herein. Actionable gene targets were analyzed for mutually exclusive or simultaneous expression.

A total of 230 GC specimens were analyzed. The median age of patients was 61 (IQR: 50-72) years with the majority being male (n=139, 60%). IHC actionable targets included: 60% (n=138) high TOP2A, 63% (n=145) negative TS, and 55% (n=127) negative ERCC1, indicating potential benefit from E, C, and F respectively. Overall, over 90% of specimens showed expression of at least one of TOP2A, TS and ERCC1, indicating sensitivity to at least one of E, C and F. When analyzing for simultaneous expression profiles of the three genes, 24% (n=55) of patients had gene expression levels that suggested sensitivity to all three agents (ECF), whereas 6.5% (n=15) of patients expressed no actionable targets demonstrating a potential lack of sensitivity to first line ECF therapy. Overall, 61% (n=140) of patients had molecular profiles that indicated sensitivity to two or more agents.

Biomarker analysis of GC suggests that 76% of patients do not possess molecular profiles that reflect complete sensitivity to standard front-line ECF therapy. Further biomarker analysis to identify actionable targets associated with alternative chemotherapies is indicated.

### Example 16: Molecular Profiling of Neuroendocrine Carcinomas Using Next Generation Sequencing

Neuroendocrine carcinomas are poorly understood and rare form of malignancies with highly variable clinical course. This Example presents a systematic analysis of 1250 cases that have been assessed by molecular profiling in a CLIA certified laboratory in order to identify biomarkers of drug sensitivity. The molecular profiling used a combination of immunohistochemistry (IHC), copy number analysis and sequencing of certain oncogenes based on their relevance to existing cancer therapies. Identification of a pathogenic pathway may provide for a druggable target in neuroendocrine tumors (NET), regardless of histologic classification or primary organ site.

Of 1250 cases, molecular profiling identified actionable alterations in 90% of analyzed cases (1130/1250). Low expression of MGMT, a potential marker of sensitivity to alkylating agents, was found in 100/219 pancreatic cases (46%). Sequencing of tumors showed mutations in: BRAF (4/369 (V600E in 3 and G596R in 1)), CTNNB1 (2/150), KIT (3/281), EGFR (1/178), FGFR2 (1/150), GNAS (1/150), HRAS (2/150), PIK3CA (6/343), RB (2/150) VHL (1/150), KRAS (10/125), NRAS (2/274), and APC (2/150). Gene amplifications found were: MET (4/236) and EGFR (46/686). Other biomarkers identified included high expression of RRM1 in 244/1100 tumors by IHC.

In several cases, dramatic responses to marker-guided therapy have been documented thus supporting the clinical relevance of molecular profiling in neuroendocrine carcinomas.

Assessment of neuroendocrine tumors with multiplatform molecular profiling revealed diverse biomarkers of drug response. Despite seemingly low frequency of individual biomarkers, the comprehensive evaluation of NET identified clinically relevant targets in the majority of patients.

### Example 17: Molecular Profiling of Gynecological Tumors

In this Example, molecular profiling is used to determine biomarker status and predict drug response in various gynecological cancers, including primary, metastatic and recurrent endometrial, ovarian and cervical cancers. Markers assessed according to the invention include without limitation phosphatidylinositide 3-kinases, HER 2, EGFR, CMET, K-RAS, BRCA, TUBB3, ER, PR, FBXW7. The markers are assessed for gene expression, protein expression, gene copy number, and/or mutational status as described herein.

### Example 18: Molecular Profiling of Advanced Refractory Prostate Cancer

Prostate cancer is the second leading cause of cancer-related death among men in the U.S. Forty percent of men diagnosed will develop metastatic disease which has few treatment options. This Example describes molecular profiling of prostate cancer tumors and potential therapeutic options.

We reviewed profiling data of over 330 patients from a large referral laboratory (Canis Life Sciences, Phoenix, Ariz.) for information on biomarkers of drug response. Multiple methodologies were employed: sequencing (Next Generation (NGS), Sanger, pyrosequencing), in-situ hybridization (fluorescent (FISH) and chromogenic (CISH)) and immunohistochemistry (IHC). High expression was observed for AR, MRP1, TOPO1, TLE3 and EGFR, with positivity rates of 89%, 87%, 63%, 48% and 47%, respectively. Low expresion was observed for TS, PGP, TUBB3, RRM1, PTEN and MGMT, with negativity rates of 94%, 87%, 75%, 69%, 54% and 45%, respectively. Gene copy number increases for EGFR and cMYC were observed in 13% of patients. Sequencing data showed 48% mutation rate for TP53, 18% for PTEN, 9% for CTNNB1, 8% for PIK3CA, 5% for RB1, ATM and cMET, and ˜2% for K/HRAS, ERBB4, ALK, BRAF and cKIT. Regarding targeted therapy options, imatinib may be considered for patients with high cKIT or PDGFRA (9-10%), and cetuximab for patients with EGFR positivity (13-47%). Promising agents may be considered, including cabozantinib, based on 4% of cohort with cMET aberrations or PAM pathway inhibitors (BEZ234, everolimus) based on ˜30% of cohort with PIK3CA pathway activation. Lastly, HDAC inhibitors have recently been linked to cMYC driven cancers (13% amplified). Chemotherapies including 5-FU, gemcitabine and temozolomide may be options based on ˜70% of cohort with low TS, RRM1 or MGMT. Biomarker guidance for common prostate cancer drugs is also provided, including cabazitaxel, based on ˜70% of cohort with low TUBB3 or PGP, or high TLE3. Finally, continued dependence on androgen signaling is exhibited by 89% of cohort with high AR, indicating potential utility of anti-androgen agents like enzalutamide.

Tumor profiling identified subsets of patients that may benefit from targeted agents approved for other solid tumors (e.g., imatinib, cetuximab), promising therapies in clinical trials (e.g., cabozantinib) or agents not routinely used for prostate cancer (e.g., gemcitabine).

### Example 19: Therapeutic Implications of Ras-ERK and PI3k-mTOR Pathway Profiling in Solid Tumors

Ras-ERK and PI3K-mTOR pathways are key regulators of cell proliferation, differentiation, survival, migration and metabolism. Alterations of these pathways are commonly seen in cancer pathogenesis. As Next Generation Sequencing (NGS) platforms become more accessible to physicians in clinical care settings, the use of highly multiplexed mutational analysis for personalized medicine is on the rise. Molecular profiling of multiple signaling pathways can provide a basis for selecting targeted single agents or combination cancer therapy for treating cancer patients.

In this Example, biomarker components of the Ras-ERK pathway were tested by NGS in a cohort of tumor samples. Genes assessed by NGS included KRAS, NRAS, HRAS and BRAF. Genes involved in the PI3K-mTOR pathway tested by NGS included PIK3CA, PTEN, AKT1 and STK11. NGS was performed using the Trueseq Amplicon Cancer Panel using Illumina's Miseq platform (Illumina Corp., San Diego, Calif.). Formalin-fixed paraffin-embedded tissue sections from 2520 patients were subjected to DNA extraction and NGS. Immunohistochemistry (IHC) using anti-PTEN clone 6H2.1 (Dako North America, Inc., Carpinteria, Calif. 93013) was used to analyze PTEN protein expression.

Among 2520 cancer samples, a higher frequency of mutations in the mTOR pathway over that of ERK was observed for breast cancer (56% cases mutated in the mTOR pathway vs 0.70% cases mutated in the ERK pathway), endometrial cancer (52.8% mTOR vs 2.8% ERK), ovarian surface epithelial carcinoma (21.7% mTOR vs 6.8% ERK), which may explain the success of mTOR inhibitors in these female prevalent/restricted cancers. Significant bias towards ERK pathway was observed for pancreatic adenocarcinoma (4.9% mTOR vs 51.0% ERK), and a near significant trend towards the ERK pathway was seen for melanoma (12.9% mTOR vs 29.7% ERK). Colorectal adenocarcinoma and pancreatic adenocarcinoma were more likely to have alterations in both ERK and mTOR pathways compared with other tumor types. When NGS data was used instead of IHC for PTEN analysis, there were significantly fewer cases with PTEN alterations, highlighting the potential advantage of using both NGS and IHC to evaluate PTEN status.

Pathway profiling reveals mTOR bias in female prevalent/restricted tumors and ERK bias in colorectal adenocarcinoma. Colorectal adenocarcinoma and pancreatic adenocarcinoma have a tendency to have mutations in genes of both mTOR and ERK pathways, suggesting dual mTOR and ERK inhibitor therapy might be effective in these tumor types. Success of mTOR inhibitors in breast and endometrial cancers may also be a result of the low rate of ERK pathway activation.

### Example 20: Concordance Between PTEN Protein Expression and Gene Mutations in a Large Cohort of Cancer Patients

PTEN is a tumor suppressor gene in the cancer signaling pathway downstream of EGFR. Loss of PTEN protein expression is one of the more common occurrences in human cancers, and its loss potentially reduces the benefit from trastuzumab, EGFR-targeted therapies, and mTOR inhibitors. Loss of PTEN is usually assessed with immunohistochemistry (IHC). Mutation analysis of PTEN gene has been recently introduced in clinical use. In this Example, we compared the concordance between PTEN by IHC and PTEN sequencing technologies in a large cohort of patients with various types of cancer.

1636 patients and 29 tumor types were utilized in this study. NGS was performed using the Trueseq Amplicon Cancer Panel using Illumina's Miseq platform (Illumina Corp., San Diego, Calif.) that employs 7 amplicons to sequence exons 1, 3, 6, 7, and 8 of PTEN gene Immunohistochemistry was performed using the anti-PTEN clone 6H2.1 (Dako North America, Inc., Carpinteria, Calif. 93013).

Overall, 5% of the samples contained mutations in the PTEN gene. Of the 83 variations identified, 46% were frameshift, 29% nonsense, 23% missense, 1% inframe deletion, and 1% affecting splicing. When compared to IHC results, a significantly larger number (30% or 481 out of 1636) of patients lacked PTEN protein expression (defined as less than 50% tumor cells staining positive). 26% of the samples that were called wild type by sequencing did not show PTEN expression and 32% of the samples that contained a mutation in PTEN expressed PTEN by IHC. Among PTEN mutations, the largest discrepancy was seen with missense mutations at 31%. In contrast, of the negatively stained samples, only 13% were called mutant by sequencing whereas 96% of samples that stained positive by IHC were called wild type by sequencing.

These observations reveal low correlation between sequencing and IHC results for PTEN. These data suggest that neither the IHC nor sequencing alone have a full capability to predict PTEN status, but when combined they provide a more complete assessment of PTEN status. Additional methods (methylation assays, LOH assays) can be used to further assess PTEN status in patients with cancer.

### Example 21: Practical Issues in Identifying and Communicating Incidental and Unexpected Findings Arising from Mutation Analysis Utilizing Next Generation Sequencing in Patients with Cancer

With the maturation of next generation sequencing (NGS) platforms in clinical diagnostics, there is a wealth of data that is generated in a time efficient and cost effective manner. One consequence of generating increased amounts of clinical data is the detection of incidental and/or unintended findings. A key consideration for many clinical labs is how to report or communicate these incidental findings to the ordering physician. Recently the ACMG released Guidelines for reporting incidental findings, however, these Guidelines may not meet the needs of a reference laboratory focused on molecular profiling of tumors. We report our experience with the identification of incidental and unexpected findings using NGS in over 3,000 specimens from patients with various types of cancer and we identify the need to consider modification of Guidelines on the reporting of such findings.

Mutation analysis was performed using the Truseq Amplicon Cancer Panel (Illumina) to determine the mutation status of select regions of 44 genes (detailed above, see, e.g., Table 25 and related disclosure). Ordering physicians have the ability to order mutation analysis for single genes or a combination of genes that the physician determined to be medically necessary. For all genes not reported, all mutation positive results are evaluated by a clinical geneticist to determine if the case merits further review. Mutations that have potential implications for clinical trials, potential germ line inheritance, therapeutic response guidance, or those that may help in determining a diagnosis are identified and discussed by a multidisciplinary team including geneticists, pathologists, and literature review scientists. In order to appropriately identify patients with potential germ line inheritance of a mutation, we employed several criteria that included age at cancer diagnosis, allele frequency of the mutation, and the gene that is mutated.

In our analysis of over 3,000 samples that received mutation analysis by NGS, ˜75% of cases did not report results for all 44 genes. Of those cases, we identified 9 potentially eligible for clinical trial enrollment, 11 with potential germ line inheritance, 5 with diagnostic uncertainty, and 3 with potential FDA approved therapy implications. Two of the cases associated with diagnostic uncertainty resulted in a change of diagnosis following a consultation with the ordering physician and pathologist.

Establishing a standard procedure for addressing incidental or unexpected findings in oncology will be necessary as more reference laboratories adopt NGS platforms. Using our current method of identifying incidental findings ˜1% of cases are identified for review making this procedure tenable for high throughput oncology labs.

### Example 22: Detecting EGFR Mutations in Selecting Chemotherapy for Patients with Lung Cancer

Mutation analysis of the kinase domain of EGFR (exons 18-21) is a standard recommended procedure for patients diagnosed with non-small cell lung cancer (NSCLC). NSCLC patients whose tumor harbors certain EGFR mutations have notable responses to EGFR inhibitors. Several different methodologies are available as published assays or commercially available kits and include Sanger Sequencing, allele specific PCR (ASP) and restriction fragment length polymorphism (RFLP). Differences in the design of these three assays dictate which clinically relevant mutations will be detected.

In this Example, tumor samples were assessed using various EGFR analysis methods. Sanger Sequencing of EGFR found 518 potentially clinically actionable mutations and 45 variants of unknown significance of 4307 samples tested. To assess which clinically relevant EGFR mutations will be detected by the ASP and RFLP, we performed an in silico analysis of all observed mutations against the design specification of the ASP and RFLP assays. The RFLP assay used in this assessment was developed in our laboratory and is designed to detect all G719 mutations, all exon 19 deletions, all exon 20 insertions and the specific mutations T790M, L858R, L861R and L861Q. A commercially available ASP kit designed to detect 29 mutations in the kinase domain was also analyzed in this study.

Based on the performance characteristics assumed by the RFLP and ASP assays, the analytical sensitivities for detecting clinically actionable mutations of the two methodologies are 98.8% and 86.7% respectively, when compared to Sanger Sequencing. It is notable that the RFLP assay missed the S768I mutation (0% detected) whereas the assay did not detect some G719 mutations, exon 19 deletions, and exon 20 insertions (76.3, 86.6 and 35.3% detected respectively).

Sequencing is still the preferred method of mutation detection in EGFR for NSCLC as it is the most comprehensive. However, if tumor nuclei are limited, a more sensitive method than sequencing is required and, EGFR mutation detection by RFLP would identify more of the potentially clinically relevant mutations than ASP as the ASP method would produce false negative results in 13.5% of patients expected to respond to EGFR inhibitors.

### Example 22: ERBB2 (HER2) Mutation Spectrum in Solid Tumors

The ERBB2 gene which encodes for Her2 is a major proliferative driver for several cancer types. Gene amplification and protein expression is associated with sensitivity to Her2-targeting drugs. In some types of cancer, ERBB2 mutations may be more clinically relevant than ERBB2 results measured by gene amplification or protein expression.

The mutation spectrum of ERBB2 in solid tumors is relatively unknown. The emergence of NGS methodology has enabled high throughput detection of both known and novel oncogenic mutations in human genome including the presence of activating mutations of ERBB2.

In this Example, comprehensive genomic profiling was performed on tumors from 2962 cancer patients. These include 319 breast, 346 colorectal (CRC), 358 lung (NSCLC), 299 uterine/cervical, 543 ovarian, 128 pancreatic cancers, 126 melanoma and 843 other solid tumors (e.g. glioblastoma, sarcomas, bladder carcinoma etc.) Direct sequence analysis of ERBB2 was performed on genomic DNA isolated from a formalin-fixed paraffin-embedded tumor sample using the Illumina MiSeq platform. Specific regions of the genome were amplified using the Illumina TruSeq Amplicon Cancer Hotspot panel. The HER2 protein expression and gene copy numbers were determined by immunohistochemistry and chromogenic in situ hybridization (CISH), respectively.

ERBB2 mutations in the kinase domain were detected in 30 patients (1% of all cases). These include previously published activating mutations (P780_Y781insGSP; V842I, L755S, V777L, D769Y) and several novel ones (such as 1767 F, R784C). 6 cases with coexisting HER2 amplification included: D769Y (breast), D769H (bladder), D769Y and T862A (ovary), and two cases with V777L (CRC). 25 of the 30 patients also had additional gene mutations (e.g. TP53, APC, PIK3CA, PTEN, KRAS). Five (17%) patients had ERBB2 mutation identified as the sole driver mutation, including L755S in CRC, breast and ovarian cancer, D769H in bladder cancer and D769E in NSCLC.

These data suggest that ERBB2 mutation might be a driver mutation in various solid tumors including breast, ovarian, CRC, NSCLC. Her2 protein overexpression was observed only when the ERBB2 gene was amplified (5/6 cases) but not in any of the ERBB2 mutated non-amplified cases (0/24). Activating ERBB2 mutations can coexist with ERBB2 gene amplification (6/30=20%) and with mutations in other key driver genes ( 24/30=80%).

### Example 23: Androgen Receptor Profiling in Various Tumors

In this Example, expression of the androgen receptor (AR) was queried in various tumors and correlated to expression and/or mutation of other commonly assessed cancer biomarkers. AR expression was determined using IHC or gene expression profiling (microarray and/or RT-PCR) as described herein.
1) GIST: in ˜170 cases, observed 6% with both AR positivity and c-KIT mutation
2) Kidney: 14% AR positivity in ˜550 kidney cases
3) HCC: 16% AR positivity in ˜270 HCC cases
4) Non-epithelial ovarian cancer: 26% AR positivity (˜250 non-EOC cases; 26 Leydig cases)

Lower coincidence of AR expression was observed with the following: EGFR mutation in NSCLC, cKIT mutation in GIST, and Her2 mutation or overexpression in gastric cancer.

### Example 24: EGFRvIII Mutation Detection by Fragment Analysis

EGFRvIII is a mutated form of the epidermal growth factor receptor protein (EGFR) that contains a deletion of exons 2 through 7 on the extracellular ligand binding domain, which confers ligand-independent activation of EGFR. The tumorigenicity of EGFRvIII and its tumor-specific expression make it an attractive therapeutic target, and various therapeutic agents targeting this variant are being investigated in different stages of clinical trials.

EGFRvIII Fragment Analysis uses RNA extracted from formalin-fixed paraffin-embedded (FFPE) tissues in a Reverse Transcription reaction, followed by Polymerase Chain Reaction (PCR) and subsequent capillary electrophoresis on the ABI 3500xL Genetic Analyzer.

Mutation Analysis of EGFRvIII using Fragment Analysis will be performed on FFPE tissue samples. This assay has the sensitivity to detect EGFRvIII deletions down to 20% mutation; therefore, patient tissue typically contains 20% or more, e.g., ≥50%, tumor nuclei for patient testing.

This assay detects mRNA with a deletion of EGFR exons 2-7 as well as wild-type EGFR transcripts within the exon 2-7 region. Two sets of primers in a single reaction are used to amplify cDNA of wild-type EGFR (89 base pair fragment) and EGFRvIII (98 base pair fragment). If the EGFRvIII fragment is not detected, the wild-type EGFR fragment confirms that the reaction was successful.

References, each of which is incorporated herein in its entirety:
1) Gan, H K., et al. 2009 “The EGFRvIII variant in glioblastoma multiforme” J Clin Neurosci 16(6):748-54
2) Sampson, J., et al. 2010 “Immunologic escape after prolonged progression-free survival with epidermal growth factor receptor variant III peptide vaccination in patients with newly diagnosed glioblastoma.” J Clin Oncol 28(31): 4722-9
3) Sampson, J., et al. 2011 “Greater chemotherapy-induced lymphopenia enhances tumor-specific immune responses that eliminate EGFRvIII-expressing tumor cells in patients with glioblastoma.” Neuro Oncol 13(3): 324-33
4) Scott, A A., et al. 2007 “A phase I clinical trial with monoclonal antibody ch806 targeting transitional state and mutant epidermal growth factor receptors” Proc Natl Acad Sci USA. 104(10):4071-6
5) Jeuken, J., et al. 2009 “Robust Detection of EGFR Copy Number Changes and EGFR Variant III: Technical Aspects and Relevance for Glioma Diagnostics” Brain Pathology 19: 661-671

Although preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A method of identifying one or more candidate treatment for a cancer in a subject in need thereof, compromising:
(a) determining a molecular profile for a sample from the subject by assessing a panel of gene or gene products, wherein the panel of gene or gene products are assessed as indicated in Table 21, FIG. 33A or FIG. 33B; and
(b) identifying one or more treatment that is beneficially associated with the molecular profile of the subject, and optionally one or more treatment associated with lack of benefit, according to the determining in (a) and one or more rules in Table 22, thereby identifying the one or more candidate treatment.

2.-160. (canceled)
